(19) United States
(12) Patent Application Publication
(10) Pub. No.: US 2026/0306642 A1
(43) Pub. Date: Oct. 8, 2026

(54) STAT DEGRADERS AND USES THEREOF

(71) Applicant: Recludix Pharma, Inc., San Diego, CA (US)

(72) Inventors: Howard Bregman, San Diego, CA (US); Neil Bifulco, San Diego, CA (US); Giovanni Cianchetta, San Diego, CA (US); Brian Hodous, Arlington, MA (US); Samuel K. Reznik, San Diego, CA (US); Ernest Allen Sickmier, San Diego, CA (US); Yong Tang, West Roxbury, MA (US); Andrew Tasker, San Diego, CA (US); Xia Tian, San Diego, CA (US); Rishi G. Vaswani, San Diego, CA (US); John Yeoman, San Diego, CA (US)

(21) Appl. No.: 19/479,550

(22) PCT Filed: May 8, 2024
(86) PCT No.: PCT/US2024/028326

§ 371 Date: Oct. 28, 2025

Related U.S. Application Data
(60) Provisional application No. 63/465,055, filed on May 9, 2023.

(51) Int. Cl. A61K 47/55; A61K 47/54

(52) U.S. Cl. CPC A61K 47/55; A61K 47/545; A61K 47/548

(57) ABSTRACT

The current invention provides degraders of STAT3 and/or STAT6, which are useful for treating a variety of conditions N associated with STAT3 and/or STAT6.

### I. FIELD OF THE INVENTION

The present invention relates to novel compounds for use as degraders of Signal Transducer and Activator of Transcription (STAT).

### II. BACKGROUND

The Signal Transducer and Activator of Transcription (STAT) family of proteins consists of transcription factors that play an essential role in the regulation of cell processes, such as proliferation, differentiation, apoptosis and angiogenesis. Seven STAT genes have been identified in the human genome: STAT1, STAT2, STAT3, STAT4, STAT5a, STAT5b, and STAT6.

STAT3 has received particular attention because it is strongly associated with the promotion of tumor growth and immune evasion, and the only STAT family member whose genetic deletion results in embryonic lethality. Indeed, aberrantly elevated STAT3 activity has been estimated to occur in more than 70% of human cancers. Activated STAT3 mediates critical gene expression changes and molecular events that dysregulate cell growth and apoptosis, promote angiogenesis, invasion, metastasis, and the development of resistance to apoptosis, and suppress the host's immune surveillance of the tumor, thereby making constitutively-active STAT3 a critical mediator of carcinogenesis and tumor progression.

Another STAT protein that has gained recent interest is STAT6. Recent studies have shown that STAT6 signaling is essential for IL-4- and IL-13-induced epithelial mesenchymal transition (EMT) and aggressiveness of colorectal cancer cells (CRC) cells. STAT6 is involved in several aspects of inflammatory disease and other related conditions.

Ubiquitin-Proteasome Pathway (UPP) or Ubiquitin-Proteasome System (UPS) is a critical pathway that regulates key regulator proteins and degrades misfolded or abnormal proteins. UPP is central to multiple cellular processes, and if defective or imbalanced, it leads to pathogenesis of a variety of diseases. The covalent attachment of ubiquitin to specific protein substrates is achieved through the action of E3 ubiquitin ligases.

### III. SUMMARY

Given their role in the regulation of cell processes, modulating the activity of one or more STAT proteins, particularly STAT3 and/or STAT6, represent a pivotal area of investigation for the treatment of cancer, inflammatory conditions, and other therapeutic needs.

Targeted protein degradation has become an advancing area as seen with rapid progression of PROTACs (proteolysis targeting chimeras) to clinic. PROTACs are protein degraders, which utilize the cell's own waste disposal machinery to eliminate, instead of inhibit, a target protein. PROTACs are bifunctional in that they simultaneously bind a target protein and an E3 ligase protein. This event subsequently ubiquitylates the target, marking it for proteasomal degradation. This, and related processes, represent a new therapeutic modality for the treatment of diseases.

The present invention provides novel bifunctional compounds for degradation of STAT3 and/or STAT6 by recruiting them to E3 uniquitin ligase for degradation. The advantage of using the methods of the invention are that a broad range of targets that may be modulalted by recruiting E3 ubiquitin ligase for degradation.

In certain aspects, the invention provides effective degraders of the STAT3 and/or STAT6. The degraders of STAT3 and/or STAT6 have general Formula A:

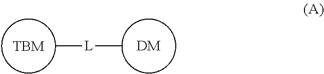

wherein, TBM is target binding moiety; DM is degradation moiety; and L is a linker between TBM and DM. A TBM is a moiety that binds to a target protein that should be degraded. In certain embodiments, the TBM is the moiety that binds to STAT3 and/or STAT6. The degradation moiety is responsible for induding degradation of the target. In certain embodiments, the degradation moiety acts by recruitment of ubiquiting E3 ligase to the target attached to TBM, and thus results in degradation of the target.

In certain aspects, the compounds of the invention provide that the TBM moiety comprises a STAT3 and/or STAT6 binding moiety. The STAT3 and/or STAT6 binding moeity are inhibitors and/or modulators of STAT3 and/or STAT6. In certain embodiments, the STAT3 and/or STAT6 binding are compounds provided in WO 2023/133336, which is incorporated by reference in its entirey.

In certain embodiments, the STAT3 and/or STAT6 binding moiety is a compound of Formula (I′):

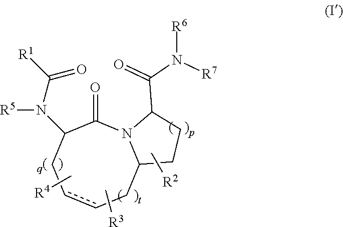

or a pharmaceutically acceptable salt thereof, wherein:
q is 0 or 1 and t is 0, 1, or 2, provided that at least one of q or t is 1;
p is 1 or 2;
the dotted line represents a single or double bond;
R¹ is selected from an 8- to 10-membered fused bicyclic heteroaryl substituted with —CR^{1a}R^{2a}P(O)OR^{1b}OR^{2b} or —CR^{1a}R^{2a}P(O)[OR^{1b}][NH(AA)C(O)OR^{T}], an 8- to 10-membered fused bicyclic heterocyclyl substituted with —CR^{1a}R^{2a}P(O)OR^{1b}OR^{2b} or —CR^{1a}R^{2a}P(O)[OR^{1b}][NH(AA)C(O)OR^{T}], an aryl substituted with CR^{1a}R^{2a}P(O)OR^{1b}OR^{2b} or —CR^{1a}R^{2a}P(O)[OR^{1b}][NH(AA)C(O)OR^{T}], a —(C₁-C₄)alkyl(aryl) wherein said aryl portion of —(C₁-C₄)alkyl(aryl) is substituted with —CR^{1a}R^{2a}P(O)OR^{1b}OR^{2b} or —CR^{1a}R^{2a}P(O)[OR^{1b}][NH(AA)C(O)OR^{T}], and a —(C₂-C₄)alkenyl(aryl) wherein said aryl portion of —(C₂-C₄)alkenyl(aryl) is substituted with —CR^{1a}R^{2a}P(O)OR^{1b}OR^{2b} or —CR^{1a}R^{2a}P(O)[OR^{1b}][NH(AA)C(O)OR^{T}];
R^{1a} and R^{2a} are each absent or are independently selected from hydrogen, cyano, (C₁-C₄)alkyl, hydroxy(C₁-C₄)alkyl and fluoro; or R^{1a} and R^{2a} taken together with the carbon they are attached form oxo;
R^{1b} and R^{2b} are each absent or are independently selected from hydrogen, (C₁-C₄)alkyl, halo(C₁-C₄)alkyl, —[(C₁-C₄)alkyl]-OC(O)—[(C₁-C₄)alkyl], —[(C₁-C₄)alkyl]-C(O)O—[(C₁-C₄)alkyl], —[(C₁-C₄)alkyl]-O—[(C₁-C₂₀)alkyl], —[(C₁-C₄)alkyl]-OC(O)-[halo(C₁-C₄)alkyl], [(C₁-C₄)alkyl]-OC(O)O—[5- to 7-membered heterocyclyl], [(C₁-C₄)alkyl]-OC(O)-[5- to 7-membered heterocyclyl], —[(C₁-C₄)alkyl]-OC(O)—[(C₁-C₄)alkyl]-OH, —[(C₁-C₄)alkyl]-OC(O)—[(C₁-C₄)alkyl]-O—[(C₁-C₄)alkyl], —[(C₁-C₄)alkyl]-OC(O)O—[(C₁-C₄)alkyl], —[(C₁-C₄)alkyl]-OC(O)O-[halo(C₁-C₄)alkyl], —[(C₁-C₄)alkyl]-OC(O)O—[(C₁-C₄)alkyl]-OH, —[(C₁-C₄)alkyl]-OC(O)O—[(C₁-C₄)alkyl]-O—[(C₁-C₄)alkyl], —[(C₁-C₄)alkyl]-SC(O)—[(C₁-C₄)alkyl], —[(C₁-C₄)alkyl]-SC(O)-[halo(C₁-C₄)alkyl], —[(C₁-C₄)alkyl]-SC(O)—[(C₁-C₄)alkyl]-OH, —[(C₁-C₄)alkyl]-SC(O)—[(C₁-C₄)alkyl]-O—[(C₁-C₄)alkyl], —[(C₁-C₄)alkyl]-OC(O)NH(C₁-C₄)alkyl], —[(C₁-C₄)alkyl]-OC(O)N[(C₁-C₄)alkyl]2, 5- to 6-membered heteroaryl, and aryl, wherein said 5- to 6-membered heteroaryl and aryl are each optionally and independently substituted with, as valency permits, 1 to 2 groups selected from halo, cyano, and (C₁-C₄)alkyl and wherein said 5- to 7-membered heterocyclyl of [(C₁-C₄)alkyl]-OC(O)O—[5- to 7-membered heterocyclyl] and [(C₁-C₄)alkyl]-OC(O)-[5- to 7-membered heterocyclyl] are each optionally and independently substituted with, as valency permits 1 to 2 groups selected from C(O)OR^{h};
R² is selected from hydrogen, halo, (C₁-C₄)alkyl, halo(C₁-C₄)alkyl, (C₁-C₄)alkoxy, halo(C₁-C₄)alkoxy, hydroxy(C₁-C₄)alkyl, cyano, and hydroxyl;
R³ and R⁴ are each independently selected from hydrogen, halo, (C₁-C₄)alkyl, halo(C₁-C₄)alkyl, hydroxy(C₁-C₄)alkyl, —(C₁-C₄)alkylphenyl, (C₁-C₄)alkoxy, halo(C₁-C₄)alkoxy, —(C₁-C₄)alkyl(C₁-C₄)alkoxy, hydroxyl, cyano, —NR^{a}R^{b}, phenyl, (C₃-C₆)cycloalkyl, 5- to 6-membered heteroaryl, and 4- to 6-membered heterocyclyl, wherein said phenyl, (C₃-C₆)cycloalkyl, 5- to 6-membered heteroaryl, and 4- to 6-membered heterocyclyl are each optionally substituted with, as valency permits, 1 to 3 groups selected from R^{S};
or R³ and R⁴ are taken together on the same carbon atom to form a (C₃-C₆)cycloalkyl or a 4- to 6-membered heterocyclyl each optionally substituted with, as valency permits, 1 to 3 groups selected from halo, (C₁-C₄)alkyl, halo(C₁-C₄)alkyl, (C₁-C₄)alkoxy, and halo(C₁-C₄)alkoxy;
R⁵ and R⁶ are each independently selected from hydrogen and (C₁-C₄)alkyl;
R⁷ is selected from (C₁-C₄)alkyl, phenyl, 4- to 9-membered monocyclic or bicyclic heterocyclyl, and 5- to 10-membered monocyclic or bicyclic heteroaryl, wherein said (C₁-C₄)alkyl is optionally substituted with, as valency permits, 1 to 3 groups selected from R^{Y} and said phenyl, 4- to 9-membered monocyclic or bicyclic heterocyclyl, and 5- to 10-membered monocyclic or bicyclic heteroaryl are each optionally substituted with, as valency permits, 1 to 3 groups selected from R^{Z}; or
R⁶ and R⁷ together with the nitrogen atom to which they are attached form a 4- to 14-membered monocyclic or bicyclic heterocyclyl or a 5- to 12-membered monocyclic or bicyclic heteroaryl, each of which being optionally substituted with, as valency permits, 1 to 3 groups selected from R^{Q};
AA is the residue of an alpha or beta natural or non-natural amino acid;
R^{T} is selected from (C₁-C₄)alkyl, benzyl, and phenyl, wherein said phenyl is optionally substituted with 1 or 2 groups selected from halo, (C₁-C₄)alkyl, and halo(C₁-C₄)alkyl;
R^{Q} is selected from halo, (C₂-C₄)alkenyl, (C₁-C₄)alkyl, (C₁-C₄)alkoxy, halo(C₁-C₄)alkoxy, cyano, phenyl, hydroxyl, 4- to 6-membered heterocyclyl, 5- to 10-membered monocyclic or bicyclic heteroaryl, (C₃-C₆)cycloalkyl, oxo, imino, —O(phenyl), —C(O)R^{g}, —C(O)OR^{e}, —NHC(O)R^{e}, —C(O)NR^{c}R^{d}, —NR^{a}R^{b}, —S(O)R^{e}R^{f}, —S(O)₂R^{f}, —S(O)═NH(C₁-C₄)alkyl, —S(O)NR^{e}R^{f}, and —S(O)₂NR^{e}R^{f}, wherein said (C₂-C₄)alkenyl and (C₁-C₄)alkyl are each optionally and independently substituted with, as valency permits, 1 to 3 groups selected from R^{M}, and wherein said phenyl, 5- to 10-membered monocyclic or bicyclic heteroaryl, (C₃-C₆)cycloalkyl, and 4- to 6-membered heterocyclyl are each optionally and independently substituted with, as valency permits, 1 to 3 groups selected from R^{F};
R^{Y} is selected from halo, (C₁-C₄)alkoxy, halo(C₁-C₄)alkoxy, cyano, —C(O)R^{g}, —C(O)OR^{e}, —NHC(O)R^{e}, —NR^{a}R^{b}, —S(O)R^{e}R^{f}, —S(O)₂R^{f}, —S(O)NR^{e}R^{f}, —S(O)═NH(C₁-C₄)alkyl, —S(O)₂NR^{e}R^{f}, hydroxyl, phenyl, 4- to 6-membered heterocyclyl, and 5- to 10-membered monocyclic or bicyclic heteroaryl, wherein said phenyl, 4- to 6-membered heterocyclyl, and 5- to 10-membered monocyclic or bicyclic heteroaryl are each optionally substituted with, as valency permits, 1 to 3 groups selected from R^{X};
R^{J} and R^{M} are each independently selected from halo, (C₁-C₄)alkoxy, halo(C₁-C₄)alkoxy, cyano, —C(O)R^{g}, —C(O)OR^{e}, —NHC(O)R^{e}, —C(O)NR^{c}R^{d}, —NR^{a}R^{b}, —S(O)R^{e}R^{f}, —S(O)₂R^{f}, —S(O)NR^{e}R^{f}, —S(O)═NH(C₁-C₄)alkyl, —S(O)₂NR^{e}R^{f}, hydroxyl, phenyl, 4- to 6-membered heterocyclyl, and 5- to 10-membered monocyclic or bicyclic heteroaryl, wherein said phenyl, 4- to 6-membered heterocyclyl, and 5- to 10-membered monocyclic or bicyclic heteroaryl are each optionally substituted with, as valency permits, 1 to 3 groups selected from R^{X};
R^{F}, R^{S}, R^{X}, and R^{Z} are each independently selected from halo, cyano, (C₁-C₄)alkyl, halo(C₁-C₄)alkyl, —(C₁-C₄)alkyl(C₁-C₄)alkoxy, hydroxy(C₁-C₄)alkyl, —(C₁-C₄)alkylphenyl, (C₂-C₄)alkenyl, halo(C₂-C₄)alkenyl, (C₂-C₄)alkynyl, halo(C₂-C₄)alkynyl, (C₁-C₄)alkoxy, halo(C₁-C₄)alkoxy, hydroxyl, oxo, imino, phenyl, —S(O)R^{e}R^{f}, —S(O)₂R^{f}, —S(O)═NH(C₁-C₄)alkyl, —S(O)NR^{e}R^{f}, and —S(O)₂NR^{e}R^{f}, —C(O)OR^{e}, —NR^{c}C(O)R^{e}, —C(O)R^{g}, —C(O)NR^{c}R^{d}, and —NR^{a}R^{b}, wherein said phenyl and said phenyl for the group —(C₁-C₄)alkylphenyl are each optionally and independently substituted with, as valency permits 1 to 3 groups selected from halo, cyano, (C₁-C₁₀)alkyl, (C₂-C₁₀)alkenyl, (C₂-C₁₀)alkynyl, halo(C₁-C₁₀)alkyl, (C₁-C₁₀)alkoxy, and halo(C₁-C₁₀)alkoxy, wherein said (C₁-C₁₀)alkyl, (C₂-C₁₀)alkenyl and (C₂-C₁₀)alkynyl are each optionally substituted with, as valency permits a 5- to 10-membered monocyclic or bicyclic heteroaryl or a 4- to 10-membered monocyclic or bicyclic heterocyclyl each of said 5- to 10-membered monocyclic and bicyclic heteroaryl or a 4- to 10-membered monocyclic or bicyclic heterocyclyl being optionally substituted with oxo or a 5- to 7-membered heterocyclyl that is optionally substituted with 1 to 2 oxo; and
R^{a}, R^{b}, R^{c}, R^{d}, R^{e}, R^{f}, R^{g}, and R^{h} are each independently selected from, as valency permits, hydrogen, (C₁-C₄)alkyl, phenyl, (C₃-C₆)cycloalkyl, 4- to 6-membered heterocyclyl and 5- to 6-membered heteroaryl, wherein said (C₁-C₄)alkyl is optionally substituted with, as valency permits, 1 to 3 groups selected from R^{J}, and said phenyl, (C₃-C₆)cycloalkyl, 4- to 6-membered heterocyclyl, and 5- to 6-membered heteroaryl are each independently optionally substituted with, as valency permits, 1 to 3 groups selected from halo, cyano, (C₁-C₄)alkyl, halo(C₁-C₄)alkyl, (C₁-C₄)alkoxy, halo(C₁-C₄)alkoxy, hydroxyl, phenyl, and benzyl.

In certain embodiments from the present invention, the L and DM moieties are described herein. In certain embodiments, the L and DM moieties are provided in WO 2024/064080, which is incorporated by reference in its entirety.

In certain aspects, the invention provides that the compounds of the invention are compounds having the structural Formula I:

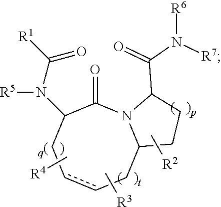

or a pharmaceutically acceptable salt thereof, wherein:
q is 0 or 1 and t is 0, 1, or 2, provided that at least one of q or t is 1;
p is 1 or 2;
the dotted line represents a single or double bond;
R¹ is selected from an 8- to 10-membered fused bicyclic heteroaryl substituted with —CR^{1a}R^{2a}P(O)OR^{1b}OR^{2b} or —CR^{1a}R^{2a}P(O)[OR^{1b}][NH(AA)C(O)OR^{T}], an 8- to 10-membered fused bicyclic heterocyclyl substituted with —CR^{1a}R^{2a}P(O)OR^{1b}OR^{2b} or —CR^{1a}R^{2a}P(O)[OR^{1b}][NH(AA)C(O)OR^{T}], an aryl substituted with CR^{1a}R^{2a}P(O)OR^{1b}OR^{2b} or —CR^{1a}R^{2a}P(O)[OR^{1b}][NH(AA)C(O)OR^{T}], a —(C₁-C₄)alkyl(aryl) wherein said aryl portion of —(C₁-C₄)alkyl(aryl) is substituted with —CR^{1a}R^{2a}P(O)OR^{1b}OR^{2b} or -CR^{1a}R^{2a}P(O)[OR^{1b}][NH(AA)C(O)OR^{T}], and a —(C₂-C₄)alkenyl(aryl) wherein said aryl portion of —(C₂-C₄)alkenyl(aryl) is substituted with —CR^{1a}R^{2a}P(O)OR^{1b}OR^{2b} or —CR^{1a}R^{2a}P(O)[OR^{1b}][NH(AA)C(O)OR^{T}];
R^{1a} and R^{2a} are each absent or are independently selected from hydrogen, cyano, (C₁-C₄)alkyl, hydroxy(C₁-C₄)alkyl and fluoro; or R^{1a} and R^{2a} taken together with the carbon they are attached form oxo;
R^{1b} and R^{2b} are each absent or are independently selected from hydrogen, (C₁-C₄)alkyl, halo(C₁-C₄)alkyl, —[(C₁-C₄)alkyl]-OC(O)—[(C₁-C₄)alkyl], —[(C₁-C₄)alkyl]-C(O)O—[(C₁-C₄)alkyl], —[(C₁-C₄)alkyl]-O—[(C₁-C₂₀)alkyl], —[(C₁-C₄)alkyl]-OC(O)-[halo(C₁-C₄)alkyl], [(C₁-C₄)alkyl]-OC(O)O—[5- to 7-membered heterocyclyl], [(C₁-C₄)alkyl]-OC(O)-[5- to 7-membered heterocyclyl], —[(C₁-C₄)alkyl]-OC(O)—[(C₁-C₄)alkyl]-OH, —[(C₁-C₄)alkyl]-OC(O)—[(C₁-C₄)alkyl]-O—[(C₁-C₄)alkyl], —[(C₁-C₄)alkyl]-OC(O)O—[(C₁-C₄)alkyl], —[(C₁-C₄)alkyl]-OC(O)O—[halo(C₁-C₄)alkyl], —[(C₁-C₄)alkyl]-OC(O)O—[(C₁-C₄)alkyl]-OH, —[(C₁-C₄)alkyl]-OC(O)O—[(C₁-C₄)alkyl]-O—[(C₁-C₄)alkyl], —[(C₁-C₄)alkyl]-SC(O)—[(C₁-C₄)alkyl], —[(C₁-C₄)alkyl]-SC(O)-[halo(C₁-C₄)alkyl], —[(C₁-C₄)alkyl]-SC(O)—[(C₁-C₄)alkyl]-OH, —[(C₁-C₄)alkyl]-SC(O)—[(C₁-C₄)alkyl]-O—[(C₁-C₄)alkyl], —[(C₁-C₄)alkyl]-OC(O)NH(C₁-C₄)alkyl], —[(C₁-C₄)alkyl]-OC(O)N[(C₁-C₄)alkyl]2, 5- to 6-membered heteroaryl, and aryl, wherein said 5- to 6-membered heteroaryl and aryl are each optionally and independently substituted with, as valency permits, 1 to 2 groups selected from halo, cyano, and (C₁-C₄)alkyl and wherein said 5- to 7-membered heterocyclyl of [(C₁-C₄)alkyl]-OC(O)O—[5- to 7-membered heterocyclyl] and [(C₁-C₄)alkyl]-OC(O)-[5- to 7-membered heterocyclyl] are each optionally and independently substituted with, as valency permits 1 to 2 groups selected from C(O)OR^{h};
R² is selected from hydrogen, halo, (C₁-C₄)alkyl, halo(C₁-C₄)alkyl, (C₁-C₄)alkoxy, halo(C₁-C₄)alkoxy, hydroxy(C₁-C₄)alkyl, cyano, and hydroxyl;
R³ and R⁴ are each independently selected from hydrogen, halo, (C₁-C₄)alkyl, halo(C₁-C₄)alkyl, hydroxy(C₁-C₄)alkyl, —(C₁-C₄)alkylphenyl, (C₁-C₄)alkoxy, halo(C₁-C₄)alkoxy, —(C₁-C₄)alkyl(C₁-C₄)alkoxy, hydroxyl, cyano, —NR^{a}R^{b}, phenyl, (C₃-C₆)cycloalkyl, 5- to 6-membered heteroaryl, and 4- to 6-membered heterocyclyl, wherein said phenyl, (C₃-C₆)cycloalkyl, 5- to 6-membered heteroaryl, and 4- to 6-membered heterocyclyl are each optionally substituted with, as valency permits, 1 to 3 groups selected from R^{S};
or R³ and R⁴ are taken together on the same carbon atom to form a (C₃-C₆)cycloalkyl or a 4- to 6-membered heterocyclyl each optionally substituted with, as valency permits, 1 to 3 groups selected from halo, (C₁-C₄)alkyl, halo(C₁-C₄)alkyl, (C₁-C₄)alkoxy, and halo(C₁-C₄)alkoxy;
R¹ and R⁶ are each independently selected from hydrogen and (C₁-C₄)alkyl;
R⁷ is selected from E, -R^{10A}E, (C₁-C₄)alkyl, phenyl, 4- to 9-membered monocyclic or bicyclic heterocyclyl, and 5- to 10-membered monocyclic or bicyclic heteroaryl, wherein said (C₁-C₄)alkyl is optionally substituted with, as valency permits, 1 to 3 groups selected from R^{Y} and said phenyl, 4- to 9-membered monocyclic or bicyclic heterocyclyl, and 5- to 10-membered monocyclic or bicyclic heteroaryl are each optionally substituted with, as valency permits, R¹¹ and/or 1 to 3 groups selected from R^{Z}; or
R⁶ and R⁷ together with the nitrogen atom to which they are attached form a 4- to 14-membered monocyclic or bicyclic heterocyclyl or a 5- to 12-membered monocyclic or bicyclic heteroaryl, each of which being optionally substituted with, as valency permits, R¹² and/or 1 to 3 groups selected from R^{Q};
R^{10A}, R^{10B}, R^{10C}, and R^{10D} are each independently a chemical spacer unit; R¹¹ is selected from —NHC(O)R^{10B}E, —OR^{10B}E, phenyl, (C₃-C₆)cycloalkyl, 5- to 6-membered heteroaryl, and 4- to 6-membered heterocyclyl each of said phenyl, (C₃-C₆)cycloalkyl, 5- to 6-membered heteroaryl, and 4- to 6-membered heterocyclyl being substituted with —OR^{10B}E, —NHR^{10B}E, —[N(C₁-C₄)alkyl]R^{10B}E, or R^{10B}E, and wherein said 4- to 6-membered heterocyclyl is optionally substituted further with oxo;
R¹² is selected from —NHC(O)R^{10C}E, —OR^{10C}E, phenyl, (C₃-C₆)cycloalkyl, 5- to 6-membered heteroaryl, and 4- to 6-membered heterocyclyl each of said phenyl, (C₃-C₆)cycloalkyl, 5- to 6-membered heteroaryl, and 4- to 6-membered heterocyclyl being substituted with —OR^{10C}E, —NHR^{10C}E, —[N(C₁-C₄)alkyl]R^{10C}E, or R^{10C}E, and wherein said 4- to 6-membered heterocyclyl is optionally substituted further with oxo;
R¹³ is selected from —NHC(O)R^{10D}E, —OR^{10D}E, phenyl, (C₃-C₆)cycloalkyl, 5- to 6-membered heteroaryl, and 4- to 6-membered heterocyclyl each of said phenyl, (C₃-C₆)cycloalkyl, 5- to 6-membered heteroaryl, and 4- to 6-membered heterocyclyl being substituted with —OR^{10D}E, —NHR^{10D}E, —[N(C₁-C₄)alkyl]R^{10D}E, or R^{10D}E, and wherein said 4- to 6-membered heterocyclyl is optionally substituted further with oxo;
E is a chemical moiety that binds to E3 ligase;
AA is the residue of an alpha or beta natural or non-natural amino acid;
R^{T} is selected from (C₁-C₄)alkyl, benzyl, and phenyl, wherein said phenyl is optionally substituted with 1 or 2 groups selected from halo, (C₁-C₄)alkyl, and halo(C₁-C₄)alkyl;
R^{Q} is selected from halo, (C₂-C₄)alkenyl, (C₁-C₄)alkyl, (C₁-C₄)alkoxy, halo(C₁-C₄)alkoxy, cyano, phenyl, hydroxyl, 4- to 6-membered heterocyclyl, 5- to 10-membered monocyclic or bicyclic heteroaryl, (C₃-C₆)cycloalkyl, oxo, imino, —O(phenyl), —C(O)R^{g}, —C(O)OR^{e}, —NHC(O)R^{e}, —C(O)NR^{c}R^{d}, —NR^{a}R^{b}, —S(O)R^{e}R^{f}, —S(O)₂R^{f}, —S(O)═NH(C₁-C₄)alkyl, —S(O)NR^{e}R^{f}, and —S(O)₂NR^{e}R^{f}, wherein said (C₂-C₄)alkenyl and (C₁-C₄)alkyl are each optionally and independently substituted with, as valency permits, 1 to 3 groups selected from R^{M}, and wherein said phenyl, 5- to 10-membered monocyclic or bicyclic heteroaryl, (C₃-C₆)cycloalkyl, and 4- to 6-membered heterocyclyl are each optionally and independently substituted with, as valency permits, 1 to 3 groups selected from R^{F};
R^{Y} is selected from halo, (C₁-C₄)alkoxy, halo(C₁-C₄)alkoxy, cyano, —C(O)R^{g}, —C(O)OR^{e}, —NHC(O)R^{e}, —NR^{a}R^{b}, —S(O)R^{e}R^{f}, —S(O)₂R^{f}, —S(O)NR^{e}R^{f}, —S(O)═NH(C₁-C₄)alkyl, —S(O)₂NR^{e}R^{f}, hydroxyl, phenyl, 4- to 6-membered heterocyclyl, and 5- to 10-membered monocyclic or bicyclic heteroaryl, wherein said phenyl, 4- to 6-membered heterocyclyl, and 5- to 10-membered monocyclic or bicyclic heteroaryl are each optionally substituted with, as valency permits, 1 to 3 groups selected from R^{X};
R^{J} and R^{M} are each independently selected from halo, (C₁-C₄)alkoxy, halo(C₁-C₄)alkoxy, cyano, —C(O)R^{g}, —C(O)OR^{e}, —NHC(O)R^{e}, —C(O)NR^{c}R^{d}, —NR^{a}R^{b}, —S(O)R^{e}R^{f}, —S(O)₂R^{f}, —S(O)NR^{e}R^{f}, —S(O)═NH(C₁-C₄)alkyl, —S(O)₂NR^{e}R^{f}, hydroxyl, phenyl, 4- to 6-membered heterocyclyl, and 5- to 10-membered monocyclic or bicyclic heteroaryl, wherein said phenyl, 4- to 6-membered heterocyclyl, and 5- to 10-membered monocyclic or bicyclic heteroaryl are each optionally substituted with, as valency permits, 1 to 3 groups selected from R^{X};
R^{F}, R^{S}, and R^{X} are each independently selected from halo, cyano, (C₁-C₄)alkyl, halo(C₁-C₄)alkyl, —(C₁-C₄)alkyl(C₁-C₄)alkoxy, hydroxy(C₁-C₄)alkyl, —(C₁-C₄)alkylphenyl, (C₂-C₄)alkenyl, halo(C₂-C₄)alkenyl, (C₂-C₄)alkynyl, halo(C₂-C₄)alkynyl, (C₁-C₄)alkoxy, halo(C₁-C₄)alkoxy, hydroxyl, oxo, imino, phenyl, —S(O)R^{e}R^{f}, —S(O)₂R^{f}, —S(O)═NH(C₁-C₄)alkyl, —S(O)NR^{e}R^{f}, and —S(O)₂NR^{e}R^{f}, —C(O)OR^{e}, —NR^{c}C(O)R^{e}, —C(O)R^{g}, —C(O)NR^{c}R^{d}, and —NR^{a}R^{b}, wherein said phenyl and said phenyl for the group —(C₁-C₄)alkylphenyl are each optionally and independently substituted with, as valency permits 1 to 3 groups selected from halo, cyano, (C₁-C₁₀)alkyl, (C₂-C₁₀)alkenyl, (C₂-C₁₀)alkynyl, halo(C₁-C₁₀)alkyl, (C₁-C₁₀)alkoxy, and halo(C₁-C₁₀)alkoxy, wherein said (C₁-C₁₀)alkyl, (C₂-C₁₀)alkenyl and (C₂-C₁₀)alkynyl are each optionally substituted with, as valency permits a 5- to 10-membered monocyclic or bicyclic heteroaryl or a 4- to 10-membered monocyclic or bicyclic heterocyclyl each of said 5- to 10-membered monocyclic and bicyclic heteroaryl or a 4- to 10-membered monocyclic or bicyclic heterocyclyl being optionally substituted with oxo or a 5- to 7-membered heterocyclyl that is optionally substituted with 1 to 2 oxo;
R^{Z} is selected from halo, cyano, (C₁-C₄)alkyl, (C₃-C₆cycloalkyl), halo(C₁-C₄)alkyl, —(C₁-C₄)alkyl(C₁-C₄)alkoxy, hydroxy(C₁-C₄)alkyl, —(C₁-C₄)alkylphenyl, —(C₁-C₄)alkylheteroaryl, (C₂-C₄)alkenyl, halo(C₂-C₄)alkenyl, (C₂-C₄)alkynyl, halo(C₂-C₄)alkynyl, (C₁-C₄)alkoxy, halo(C₁-C₄)alkoxy, —OR^{e}, oxo, imino, phenyl, 4- to 6-membered heterocyclyl, —S(O)R^{e}R^{f}, —S(O)₂R^{f}, —S(O)═NH(C₁-C₄)alkyl, —S(O)NR^{e}R^{f}, and —S(O)₂NR^{e}R^{f}, —C(O)OR^{e}, —NR^{c}C(O)R^{e}, —(C₁-C₄alkyl)C(O)R^{g}, —C(O)R^{g}, —(C₁-C₄alkyl)C(O)NR^{c}R^{d}, —C(O)NR^{c}R^{d}, —NO₂, and —NR^{a}R^{b}, wherein the (C₁-C₄)alkyl is optionally substituted with cyano, wherein said phenyl, said 4- to 6-membered heterocyclyl, and said phenyl for —(C₁-C₄)alkylphenyl are each optionally and independently substituted with, as valency permits R¹³ and/or 1 to 3 groups selected from halo, cyano, oxo, (C₁-C₁₀)alkyl, (C₂-C₁₀)alkenyl, (C₂-C₁₀)alkynyl, halo(C₁-C₁₀)alkyl, (C₁-C₁₀)alkoxy, and halo(C₁-C₁₀)alkoxy, wherein said (C₁-C₁₀)alkyl, (C₂-C₁₀)alkenyl and (C₂-C₁₀)alkynyl are each optionally substituted with, as valency permits a 5- to 10-membered monocyclic or bicyclic heteroaryl or a 4- to 10-membered monocyclic or bicyclic heterocyclyl each of said 5- to 10-membered monocyclic and bicyclic heteroaryl or a 4- to 10-membered monocyclic or bicyclic heterocyclyl being optionally substituted with oxo or a 5- to 7-membered heterocyclyl that is optionally substituted with 1 to 2 oxo;
R^{a}, R^{b}, R^{c}, R^{d}, R^{e}, R^{f}, R^{g}, and R^{h} are each independently selected from, as valency permits, hydrogen, (C₁-C₄)alkyl, phenyl, (C₃-C₆)cycloalkyl, 4- to 6-membered heterocyclyl and 5- to 6-membered heteroaryl, wherein said (C₁-C₄)alkyl is optionally substituted with, as valency permits, 1 to 3 groups selected from R^{J}, and said phenyl, (C₃-C₆)cycloalkyl, 4- to 6-membered heterocyclyl, and 5- to 6-membered heteroaryl are each independently optionally substituted with, as valency permits, 1 to 3 groups selected from halo, cyano, (C₁-C₄)alkyl, halo(C₁-C₄)alkyl, (C₁-C₄)alkoxy, halo(C₁-C₄)alkoxy, hydroxyl, phenyl, and benzyl.

In one aspect, the disclosed compounds of Formula I and pharmaceutically acceptable salts thereof act as protein degraders of STAT3 and/or STAT6, and are useful in a variety of therapeutic applications such as, for example, in treating cancer and inflammatory conditions.

Pharmaceutical compositions comprising the compounds and pharmaceutically acceptable salts of Formula I, as well as methods for their preparation are also included.

Methods of treating conditions responsive to the degradation of STAT3 and/or STAT6 using the described compounds, pharmaceutically acceptable salts, and compositions thereof are also included.

### IV. DETAILED DESCRIPTION

### 1. General Description of Compounds

The present invention provides novel bifunctional compounds for degradation of STAT3 and/or STAT6 by recruiting them to E3 uniquitin ligase for degradation. The advantage of using the methods of the invention are that a broad range of targets that may be modulalted by recruiting E3 ubiquitin ligase for degradation.

In certain aspects, the invention provides effective degraders of the STAT3 and/or STAT6. The degraders of STAT3 and/or STAT6 have general Formula A:

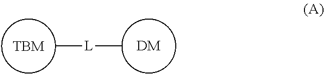

wherein, TBM is target binding moiety; DM is degradation moiety; and L is a linker between TBM and DM. A TBM is a moiety that binds to a target protein that should be degraded. In certain embodiments, the TBM is the moiety that binds to STAT3 and/or STAT6. The degradation moiety is responsible for induding degradation of the target. In certain embodiments, the degradation moiety acts by recruitment of ubiquiting E3 ligase to the target attached to TBM, and thus results in degradation of the target.

In certain aspects, the compounds of the invention provide that the TBM moiety comprises a STAT3 and/or STAT6 binding moiety. The STAT3 and/or STAT6 binding moeity are inhibitors and/or modulators of STAT3 and/or STAT6. In certain embodiments, the STAT3 and/or STAT6 binding are compounds provided in WO 2023/133336, which is incorporated by reference in its entirey.

In a first embodiment, provided herein is a compound of structural Formula I:

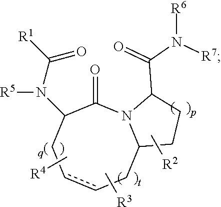

or a pharmaceutically acceptable salt thereof, wherein:
q is 0 or 1 and t is 0, 1, or 2, provided that at least one of q or t is 1;
p is 1 or 2;
the dotted line represents a single or double bond;
R¹ is selected from an 8- to 10-membered fused bicyclic heteroaryl substituted with —CR^{1a}R^{2a}P(O)OR^{1b}OR^{2b} or —CR^{1a}R^{2a}P(O)[OR^{1b}][NH(AA)C(O)OR^{T}], an 8- to 10-membered fused bicyclic heterocyclyl substituted with —CR^{1a}R^{2a}P(O)OR^{1b}OR^{2b} or —CR^{1a}R^{2a}P(O)[OR^{1b}][NH(AA)C(O)OR^{T}], an aryl substituted with CR^{1a}R^{2a}P(O)OR^{1b}OR^{2b} or —CR^{1a}R^{2a}P(O)[OR^{1b}][NH(AA)C(O)OR^{T}], a —(C₁-C₄)alkyl(aryl) wherein said aryl portion of —(C₁-C₄)alkyl(aryl) is substituted with —CR^{1a}R^{2a}P(O)OR^{1b}OR^{2b} or —CR^{1a}R^{2a}P(O)[OR^{1b}][NH(AA)C(O)OR^{T}], and a —(C₂-C₄)alkenyl(aryl) wherein said aryl portion of —(C₂-C₄)alkenyl(aryl) is substituted with —CR^{1a}R^{2a}P(O)OR^{1b}OR^{2b} or —CR^{1a}R^{2a}P(O)[OR^{1b}][NH(AA)C(O)OR^{T}];
R^{1a} and R^{2a} are each absent or are independently selected from hydrogen, cyano, (C₁-C₄)alkyl, hydroxy(C₁-C₄)alkyl and fluoro; or R^{1a} and R^{2a} taken together with the carbon they are attached form oxo;
R^{1b} and R^{2b} are each absent or are independently selected from hydrogen, (C₁-C₄)alkyl, halo(C₁-C₄)alkyl, —[(C₁-C₄)alkyl]-OC(O)—[(C₁-C₄)alkyl], —[(C₁-C₄)alkyl]-C(O)O—[(C₁-C₄)alkyl], —[(C₁-C₄)alkyl]-O—[(C₁-C₂₀)alkyl], —[(C₁-C₄)alkyl]-OC(O)-[halo(C₁-C₄)alkyl], [(C₁-C₄)alkyl]-OC(O)O—[5- to 7-membered heterocyclyl], [(C₁-C₄)alkyl]-OC(O)-[5- to 7-membered heterocyclyl], —[(C₁-C₄)alkyl]-OC(O)—[(C₁-C₄)alkyl]-OH, —[(C₁-C₄)alkyl]-OC(O)—[(C₁-C₄)alkyl]-O—[(C₁-C₄)alkyl], —[(C₁-C₄)alkyl]-OC(O)O—[(C₁-C₄)alkyl], —[(C₁-C₄)alkyl]-OC(O)O—[halo(C₁-C₄)alkyl], —[(C₁-C₄)alkyl]-OC(O)O—[(C₁-C₄)alkyl]-OH, —[(C₁-C₄)alkyl]-OC(O)O—[(C₁-C₄)alkyl]-O—[(C₁-C₄)alkyl], —[(C₁-C₄)alkyl]-SC(O)—[(C₁-C₄)alkyl], —[(C₁-C₄)alkyl]-SC(O)-[halo(C₁-C₄)alkyl], —[(C₁-C₄)alkyl]-SC(O)—[(C₁-C₄)alkyl]-OH, —[(C₁-C₄)alkyl]-SC(O)—[(C₁-C₄)alkyl]-O—[(C₁-C₄)alkyl], —[(C₁-C₄)alkyl]-OC(O)NH(C₁-C₄)alkyl], —[(C₁-C₄)alkyl]-OC(O)N[(C₁-C₄)alkyl]2, 5- to 6-membered heteroaryl, and aryl, wherein said 5- to 6-membered heteroaryl and aryl are each optionally and independently substituted with, as valency permits, 1 to 2 groups selected from halo, cyano, and (C₁-C₄)alkyl and wherein said 5- to 7-membered heterocyclyl of [(C₁-C₄)alkyl]-OC(O)O—[5- to 7-membered heterocyclyl] and [(C₁-C₄)alkyl]-OC(O)-[5- to 7-membered heterocyclyl] are each optionally and independently substituted with, as valency permits 1 to 2 groups selected from C(O)OR^{h};
R² is selected from hydrogen, halo, (C₁-C₄)alkyl, halo(C₁-C₄)alkyl, (C₁-C₄)alkoxy, halo(C₁-C₄)alkoxy, hydroxy(C₁-C₄)alkyl, cyano, and hydroxyl;
R³ and R⁴ are each independently selected from hydrogen, halo, (C₁-C₄)alkyl, halo(C₁-C₄)alkyl, hydroxy(C₁-C₄)alkyl, —(C₁-C₄)alkylphenyl, (C₁-C₄)alkoxy, halo(C₁-C₄)alkoxy, —(C₁-C₄)alkyl(C₁-C₄)alkoxy, hydroxyl, cyano, —NR^{a}R^{b}, phenyl, (C₃-C₆)cycloalkyl, 5- to 6-membered heteroaryl, and 4- to 6-membered heterocyclyl, wherein said phenyl, (C₃-C₆)cycloalkyl, 5- to 6-membered heteroaryl, and 4- to 6-membered heterocyclyl are each optionally substituted with, as valency permits, 1 to 3 groups selected from R^{S};
or R³ and R⁴ are taken together on the same carbon atom to form a (C₃-C₆)cycloalkyl or a 4- to 6-membered heterocyclyl each optionally substituted with, as valency permits, 1 to 3 groups selected from halo, (C₁-C₄)alkyl, halo(C₁-C₄)alkyl, (C₁-C₄)alkoxy, and halo(C₁-C₄)alkoxy;
R⁵ and R⁶ are each independently selected from hydrogen and (C₁-C₄)alkyl;
R⁷ is selected from E, -R^{10A}E, (C₁-C₄)alkyl, phenyl, 4- to 9-membered monocyclic or bicyclic heterocyclyl, and 5- to 10-membered monocyclic or bicyclic heteroaryl, wherein said (C₁-C₄)alkyl is optionally substituted with, as valency permits, 1 to 3 groups selected from R^{Y} and said phenyl, 4- to 9-membered monocyclic or bicyclic heterocyclyl, and 5- to 10-membered monocyclic or bicyclic heteroaryl are each optionally substituted with, as valency permits, R¹¹ and/or 1 to 3 groups selected from R^{Z}; or
R⁶ and R⁷ together with the nitrogen atom to which they are attached form a 4- to 14-membered monocyclic or bicyclic heterocyclyl or a 5- to 12-membered monocyclic or bicyclic heteroaryl, each of which being optionally substituted with, as valency permits, R¹² and/or 1 to 3 groups selected from R^{Q};
R^{10A}, R^{10B}, R^{10C}, and R^{10D} are each independently a chemical spacer unit; R¹¹ is selected from —NHC(O)R^{10B}E, —OR^{10B}E, phenyl, (C₃-C₆)cycloalkyl, 5- to 6-membered heteroaryl, and 4- to 6-membered heterocyclyl each of said phenyl, (C₃-C₆)cycloalkyl, 5- to 6-membered heteroaryl, and 4- to 6-membered heterocyclyl being substituted with —OR^{10B}E, —NHR^{10B}E, —[N(C₁-C₄)alkyl]R^{10B}E, or R^{10B}E, and wherein said 4- to 6-membered heterocyclyl is optionally substituted further with oxo;
R¹² is selected from —NHC(O)R^{10C}E, —OR^{10C}E, phenyl, (C₃-C₆)cycloalkyl, 5- to 6-membered heteroaryl, and 4- to 6-membered heterocyclyl each of said phenyl, (C₃-C₆)cycloalkyl, 5- to 6-membered heteroaryl, and 4- to 6-membered heterocyclyl being substituted with —OR^{10C}E, —NHR^{10C}E, —[N(C₁-C₄)alkyl]R^{10C}E, or R^{10C}E, and wherein said 4- to 6-membered heterocyclyl is optionally substituted further with oxo;
R¹³ is selected from —NHC(O)R^{10D}E, —OR^{10D}E, phenyl, (C₃-C₆)cycloalkyl, 5- to 6-membered heteroaryl, and 4- to 6-membered heterocyclyl each of said phenyl, (C₃-C₆)cycloalkyl, 5- to 6-membered heteroaryl, and 4- to 6-membered heterocyclyl being substituted with —OR^{10D}E, —NHR^{10D}E, —[N(C₁-C₄)alkyl]R^{10D}E, or R^{10D}E, and wherein said 4- to 6-membered heterocyclyl is optionally substituted further with oxo;
E is a chemical moiety that binds to E3 ligase;
AA is the residue of an alpha or beta natural or non-natural amino acid;
R^{T} is selected from (C₁-C₄)alkyl, benzyl, and phenyl, wherein said phenyl is optionally substituted with 1 or 2 groups selected from halo, (C₁-C₄)alkyl, and halo(C₁-C₄)alkyl;
R^{Q} is selected from halo, (C₂-C₄)alkenyl, (C₁-C₄)alkyl, (C₁-C₄)alkoxy, halo(C₁-C₄)alkoxy, cyano, phenyl, hydroxyl, 4- to 6-membered heterocyclyl, 5- to 10-membered monocyclic or bicyclic heteroaryl, (C₃-C₆)cycloalkyl, oxo, imino, —O(phenyl), —C(O)R^{g}, —C(O)OR^{e}, —NHC(O)R^{e}, —C(O)NR^{c}R^{d}, —NR^{a}R^{b}, —S(O)R^{e}R^{f}, —S(O)₂R^{f}, —S(O)═NH(C₁-C₄)alkyl, —S(O)NR^{e}R^{f}, and —S(O)₂NR^{e}R^{f}, wherein said (C₂-C₄)alkenyl and (C₁-C₄)alkyl are each optionally and independently substituted with, as valency permits, 1 to 3 groups selected from R^{M}, and wherein said phenyl, 5- to 10-membered monocyclic or bicyclic heteroaryl, (C₃-C₆)cycloalkyl, and 4- to 6-membered heterocyclyl are each optionally and independently substituted with, as valency permits, 1 to 3 groups selected from R^{F};
R^{Y} is selected from halo, (C₁-C₄)alkoxy, halo(C₁-C₄)alkoxy, cyano, —C(O)R^{g}, —C(O)OR^{e}, —NHC(O)R^{e}, —NR^{a}R^{b}, —S(O)R^{e}R^{f}, —S(O)₂R^{f}, —S(O)NR^{e}R^{f}, —S(O)═NH(C₁-C₄)alkyl, —S(O)₂NR^{e}R^{f}, hydroxyl, phenyl, 4- to 6-membered heterocyclyl, and 5- to 10-membered monocyclic or bicyclic heteroaryl, wherein said phenyl, 4- to 6-membered heterocyclyl, and 5- to 10-membered monocyclic or bicyclic heteroaryl are each optionally substituted with, as valency permits, 1 to 3 groups selected from R^{X};
R^{J} and R^{M} are each independently selected from halo, (C₁-C₄)alkoxy, halo(C₁-C₄)alkoxy, cyano, —C(O)R^{g}, —C(O)OR^{e}, —NHC(O)R^{e}, —C(O)NR^{c}R^{d}, —NR^{a}R^{b}, —S(O)R^{e}R^{f}, —S(O)₂R, —S(O)NR^{e}R^{f}, —S(O)═NH(C₁-C₄)alkyl, —S(O)₂NR^{e}R^{f}, hydroxyl, phenyl, 4- to 6-membered heterocyclyl, and 5- to 10-membered monocyclic or bicyclic heteroaryl, wherein said phenyl, 4- to 6-membered heterocyclyl, and 5- to 10-membered monocyclic or bicyclic heteroaryl are each optionally substituted with, as valency permits, 1 to 3 groups selected from R^{X};
R^{F}, R^{S}, and R^{X} are each independently selected from halo, cyano, (C₁-C₄)alkyl, halo(C₁-C₄)alkyl, —(C₁-C₄)alkyl(C₁-C₄)alkoxy, hydroxy(C₁-C₄)alkyl, —(C₁-C₄)alkylphenyl, (C₂-C₄)alkenyl, halo(C₂-C₄)alkenyl, (C₂-C₄)alkynyl, halo(C₂-C₄)alkynyl, (C₁-C₄)alkoxy, halo(C₁-C₄)alkoxy, hydroxyl, oxo, imino, phenyl, —S(O)R^{e}R^{f}, —S(O)₂R^{f}, —S(O)═NH(C₁-C₄)alkyl, —S(O)NR^{e}R^{f}, and —S(O)₂NR^{e}R^{f}, —C(O)OR^{e}, —NR^{c}C(O)R^{e}, —C(O)R^{g}, —C(O)NR^{c}R^{d}, and —NR^{a}R^{b}, wherein said phenyl and said phenyl for the group —(C₁-C₄)alkylphenyl are each optionally and independently substituted with, as valency permits 1 to 3 groups selected from halo, cyano, (C₁-C₁₀)alkyl, (C₂-C₁₀)alkenyl, (C₂-C₁₀)alkynyl, halo(C₁-C₁₀)alkyl, (C₁-C₁₀)alkoxy, and halo(C₁-C₁₀)alkoxy, wherein said (C₁-C₁₀)alkyl, (C₂-C₁₀)alkenyl and (C₂-C₁₀)alkynyl are each optionally substituted with, as valency permits a 5- to 10-membered monocyclic or bicyclic heteroaryl or a 4- to 10-membered monocyclic or bicyclic heterocyclyl each of said 5- to 10-membered monocyclic and bicyclic heteroaryl or a 4- to 10-membered monocyclic or bicyclic heterocyclyl being optionally substituted with oxo or a 5- to 7-membered heterocyclyl that is optionally substituted with 1 to 2 oxo;
R^{Z} is selected from halo, cyano, (C₁-C₄)alkyl, (C₃-C₆cycloalkyl), halo(C₁-C₄)alkyl, —(C₁-C₄)alkyl(C₁-C₄)alkoxy, hydroxy(C₁-C₄)alkyl, —(C₁-C₄)alkylphenyl, —(C₁-C₄)alkylheteroaryl, (C₂-C₄)alkenyl, halo(C₂-C₄)alkenyl, (C₂-C₄)alkynyl, halo(C₂-C₄)alkynyl, (C₁-C₄)alkoxy, halo(C₁-C₄)alkoxy, —OR^{e}, oxo, imino, phenyl, 4- to 6-membered heterocyclyl, —S(O)R^{e}R^{f}, —S(O)₂R^{f}, —S(O)═NH(C₁-C₄)alkyl, —S(O)NR^{e}R^{f}, and —S(O)₂NR^{e}R^{f}, —C(O)OR^{e}, —NR^{c}C(O)R^{e}, —(C₁-C₄alkyl)C(O)R^{g}, —C(O)R^{g}, —(C₁-C₄alkyl)C(O)NR^{c}R^{d}, —C(O)NR^{c}R^{d}, —NO₂, and —NR^{a}R^{b}, wherein the (C₁-C₄)alkyl is optionally substituted with cyano, wherein said phenyl, said 4- to 6-membered heterocyclyl, and said phenyl for —(C₁-C₄)alkylphenyl are each optionally and independently substituted with, as valency permits R¹³ and/or 1 to 3 groups selected from halo, cyano, oxo, (C₁-C₁₀)alkyl, (C₂-C₁₀)alkenyl, (C₂-C₁₀)alkynyl, halo(C₁-C₁₀)alkyl, (C₁-C₁₀)alkoxy, and halo(C₁-C₁₀)alkoxy, wherein said (C₁-C₁₀)alkyl, (C₂-C₁₀)alkenyl and (C₂-C₁₀)alkynyl are each optionally substituted with, as valency permits a 5- to 10-membered monocyclic or bicyclic heteroaryl or a 4- to 10-membered monocyclic or bicyclic heterocyclyl each of said 5- to 10-membered monocyclic and bicyclic heteroaryl or a 4- to 10-membered monocyclic or bicyclic heterocyclyl being optionally substituted with oxo or a 5- to 7-membered heterocyclyl that is optionally substituted with 1 to 2 oxo;
R^{a}, R^{b}, R^{c}, R^{d}, R^{e}, R^{f}, R^{g}, and R^{h} are each independently selected from, as valency permits, hydrogen, (C₁-C₄)alkyl, phenyl, (C₃-C₆)cycloalkyl, 4- to 6-membered heterocyclyl and 5- to 6-membered heteroaryl, wherein said (C₁-C₄)alkyl is optionally substituted with, as valency permits, 1 to 3 groups selected from R^{J}, and said phenyl, (C₃-C₆)cycloalkyl, 4- to 6-membered heterocyclyl, and 5- to 6-membered heteroaryl are each independently optionally substituted with, as valency permits, 1 to 3 groups selected from halo, cyano, (C₁-C₄)alkyl, halo(C₁-C₄)alkyl, (C₁-C₄)alkoxy, halo(C₁-C₄)alkoxy, hydroxyl, phenyl, and benzyl.

### 2. Definitions

When used in connection to describe a chemical group that may have multiple points of attachment, a hyphen (-) designates the point of attachment of that group to the variable to which it is defined. For example, —NR^{c}C(O)R^{e} means that the point of attachment for this group occurs on the nitrogen atom.

The terms “halo” and “halogen” refer to an atom selected from fluorine (fluoro, —F), chlorine (chloro, —Cl), bromine (bromo, —Br), and iodine (iodo, —I).

Unless otherwise specified, the term “alkyl” when used alone or as part of a larger moiety, such as “haloalkyl”, and the like, means saturated straight-chain or branched monovalent hydrocarbon radical.

The term “haloalkyl” includes mono, poly, and perhaloalkyl groups where the halogens are independently selected from fluorine, chlorine, bromine, and iodine.

“Alkoxy” means an alkyl radical attached through an oxygen linking atom, represented by —O-alkyl. For example, “(C₁-C₄)alkoxy” includes methoxy, ethoxy, proproxy, and butoxy.

“Haloalkoxy” is a haloalkyl group which is attached to another moiety via an oxygen atom such as, e.g., —OCHF₂ or —OCF₃.

The term “oxo” means the group ═O.

The term “imino” means the group ═NH.

Unless otherwise specified, the term “heteroaryl” refers to a 5- to 12-membered aromatic radical containing 1-4 heteroatoms selected from N, O, and S. In some instances, nitrogen atoms in a heteroaryl may be quaternized. The term “heteroaryl” may be used interchangeably with the terms “heteroaryl ring”, “heteroaryl group”, or “heteroaromatic”. A heteroaryl group may be mono- or bi-cyclic. Monocyclic heteroaryl includes, for example, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, etc. Bi-cyclic heteroaryls include groups in which a monocyclic heteroaryl ring is fused to one or more aryl or heteroaryl rings. Nonlimiting examples include indolyl, benzooxazolyl, benzooxodiazolyl, indazolyl, benzimidazolyl, benzthiazolyl, benzothiopheneyl, quinolinyl, quinazolinyl, quinoxalinyl, pyrrolopyridinyl, pyrrolopyrimidinyl, pyrrolopyridinyl, thienopyridinyl, thienopyrimidinyl, indolizinyl, purinyl, cinnolinyl, naphthyridinyl, and pteridinyl. It will be understood that when specified, optional substituents on a heteroaryl group may be present on any substitutable position and, include, e.g., the position at which the heteroaryl is attached (where valency permits).

Unless otherwise specified, the term “heterocyclyl” means a 4- to 12-membered saturated or partially unsaturated heterocyclic ring containing 1 to 4 heteroatoms independently selected from N, O, and S. The terms “heterocycle”, “heterocyclyl”, “heterocyclyl ring”, “heterocyclic group”, “heterocyclic moiety”, and “heterocyclic radical”, are used interchangeably herein. A heterocyclyl ring can be attached to its pendant group at any heteroatom or carbon atom that results in a stable structure. A heterocyclyl group may be mono- or bicyclic (e.g., a bridged, fused, or spiro bicyclic ring). Examples of monocyclic saturated or partially unsaturated heterocyclic radicals include, without limitation, azetidinyl, tetrahydrofuranyl, tetrahydrothienyl, terahydropyranyl, pyrrolidinyl, pyrrolidonyl, piperidinyl, oxazolidinyl, piperazinyl, dioxanyl, dioxolanyl, morpholinyl, dihydrofuranyl, dihydropyranyl, dihydropyridinyl, tetrahydropyridinyl, dihydropyrimidinyl, tetrahydropyrimidinyl, dihydrooxadizolyl, and dihydroisoxazolyl. Bi-cyclic heterocyclyl groups include, e.g., unsaturated heterocyclic radicals fused to another unsaturated heterocyclic radical, cycloalkyl, aryl, or heteroaryl ring, such as for example, benzodioxolyl, dihydrobenzodioxinyl, dihydrobenzofuranyl, dihydrobenzothiophenyl, 5-oxa-2,6-diazaspiro[3.4]oct-6-enyl, 6-thia-2,7-diazaspiro[3.4]octanyl, 2,6-diazaspiro[3.3]heptanyl, spiro[indoline-3,3 [symbol] -pyrrolidine]-yl, thiochromanyl, and the like. It will be understood that when specified, optional substituents on a heterocyclyl group may be present on any substitutable position and, include, e.g., the position at which the heterocyclyl is attached (where valency permits).

The term “spiro” refers to two rings that shares one ring atom (e.g., carbon).

The term “fused” refers to two rings that share two adjacent ring atoms with one another.

The term “bridged” refers to two rings that share three adjacent ring atoms with one another.

The term “aryl” refers to an aromatic carbocyclic single ring or two fused ring system containing 6 to 10 carbon atoms. Examples include phenyl, indanyl, tetrahydronaphthalene, and naphthyl. In one aspect, the aryl is phenyl or naphthyl.

The terms “cycloalkyl”, used alone or as part of a larger moiety, refers to a saturated cyclic aliphatic monocyclic or bicyclic ring system, as described herein, having from, unless otherwise specified, 3 to 10 carbon ring atoms. Monocyclic cycloalkyl groups include, without limitation, cyclopropyl, cyclobutyl, cyclopentyl, cyclopentenyl, cyclohexyl, cyclohexenyl, cycloheptyl, cycloheptenyl, and cyclooctyl. It will be understood that when specified, optional substituents on a cycloalkyl or cycloaliphatic group may be present on any substitutable position and, include, e.g., the position at which the cycloalkyl group is attached.

The term alkylene refers to a refers to a bivalent radical of a saturated, straight or branched chain hydrocarbon (e.g., CH₂) of the specified length.

The term alkynylene refers to an alkylene group that has at least one triple bond within the chain e.g., —(CH₂)₅—CH—.

The “residue of an amino acid” is the moiety remaining after formation of a bond between a reactive group in another compound (e.g., an amino group) and the carboxylic acid in the amino acid, after formation of a bond between a reactive group in another compound (e.g., a carboxylic acid) and the amino group in the amino acid, or both. As a consequence of the bond(s) formation, the carboxylic acid in the amino acid no longer has the OH group and instead has a bond between the carbonyl group and the reactive group in the compound; the amino group has only one hydrogen atom and instead has a bond between the reactive group in the other compound and the nitrogen of the amino group; or both. For example, the “residue of an alpha amino acid” can be depicted structurally as NH₂CR′R—C(O)—, —NHCR′R—C(O)OH or —NHCR′R—C(O)—; and the “residue of an beta amino acid” can be depicted structurally as or NH₂CR′RCH₂—C(O)—, —NHCR′RCH₂—C(O)OH or —NHCR′RCH₂—C(O)—, where R′ is H or C₁-C₆ alkyl and R is H or C₁-C₆ alkyl optionally substituted with 1 to 3 groups selected from halo, (C₁-C₃)alkoxy, OH, NH₂, —NH(C₁-C₄ alkyl), —N[(C₁-C₄ alkyl)]₂, SH, S(C₁-C₄ alkyl), imino, COOH, —COO(C₁-C₄ alkyl), —CO(C₁-C₄ alkyl), —CONH(C₁-C₄ alkyl)phenyl, phenyl, and 5- to 10-membered heteroaryl, wherein said C₁-C₆ alkyl may also be optionally interrupted by a sulfur or nitrogen heteroatom and wherein said phenyl is optionally substituted with 1 to 3 groups selected from OH, cyano, (C₁-C₄ alkyl), and halo(C₁-C₄ alkyl); or R is taken together with the nitrogen atoms from the alpha or beta amino acid residue to form a 4- to 6-membered heterocyclyl. For naturally occurring alpha amino acid (i.e., amino acids that occur in nature), R′ is H and R is selected from hydrogen, methyl, isopropyl, —CH₂CH(CH₃)₂, —(CH₂)₂SCH₃, —CH(CH₃)(CH₂CH₃), CH₂OH, —CH(OH)(CH₃), CH₂SH, —CH₂C(O)NH₂, —(CH₂)₂C(O)NH₂, benzyl, p-hydroxybenzyl, —CH₂(indolyl), —(CH₂)₄NH₂, —(CH₂)₃NHC(═NH₂)NH₂, —CH₂(imidazolyl), —(CH₂)COOH, and —(CH₂)₂COOH; or R taken together with the nitrogen atom of the alpha or beta amio acid residue forms a pyrrolidinyl ring.

Non-natural amino acids are known in the art and include e.g., alpha-alkyl amino acids (e.g., alpha methyl), alpha-alkylalkoxy amino acids (e.g., alpha —CH₂OCH₃), N-methyl amino acids, homo-amino acids, etc.

A chemical spacer unit refers to refers to a cleavable or non-cleavable that joins the E3 ligase binder to the remainder of the molecule described herein. In one aspect, the chemical spacer is uncleavable in vivo. In one aspect, the chemical spacer comprises one or more cyclic ring systems. In another aspect, the chemical spacer comprises an alkyl chain optionally substituted by and/or interrupted with one or more chemical groups. In one aspect, the chemical spacer comprises optimal spatial and chemical properties to effectuate optimal therapeutic activity.

A chemical moiety that binds to E3 ligase refers to a chemical structure that binds to the pocket or surface of E3 ligase. E3 ligase binders are known in the art and include, but are not limited to CRBN, VHL, IAP, or MDM2 based E3 binders. See e.g., Angewandte Chemie International Edition 59(36), May 2020, DOI:10.1002/anie.202004310; Medicinal Chemistry Communication 10(10), August 2019, DOI: 10.1039/C9MD00272C; and Molecules 2022, 27(19), 6515; https://doi.org/10.3390/molecules27196515.

Compounds having one or more chiral centers can exist in various stereoisomeric forms. Stereoisomers are compounds that differ only in their spatial arrangement. Stereoisomers include all diastereomeric, enantiomeric, and epimeric forms as well as racemates and mixtures thereof. A “geometric isomer” refers to isomers that differ in the orientation of substituent group in relationship to a carbon-carbon double bond, a cycloalkyl ring, or a bridged bicyclic system. Atoms (other than H) on each side of a carbon-carbon double bond may be in an E (substituents are on opposite sides of the carbon-carbon double bond) or Z (substituents are oriented on the same side) configuration. “Cis” refers to substituents oriented on the same side of the ring, whereas “trans” refers to substituents oriented on opposite sides of the ring.

When the stereochemical configuration at a chiral center in a compound having one or more chiral centers is depicted by its chemical name (e.g., where the configuration is indicated in the chemical name by “R” or “S”) or structure (e.g., the configuration is indicated by “wedge” bonds), the enrichment of the indicated configuration relative to the opposite configuration is greater than 50%, 60%, 70%, 80%, 90%, 99% or 99.9%. “Enrichment of the indicated configuration relative to the opposite configuration” is a mole percent and is determined by dividing the number of compounds with the indicated stereochemical configuration at the chiral center(s) by the total number of all of the compounds with the same or opposite stereochemical configuration in a mixture.

When a geometric isomer is depicted by name or structure, the enrichment of the indicated isomer relative to the opposite isomer is greater than 50%, 60%, 70%, 80%, 90%, 99% or 99.9%. “Enrichment of the indicated isomer relative to the opposite isomer” is a mole percent and is determined by dividing the number of compounds with the indicated geometrical configuration by the total number of all of the compounds with the same or opposite geometrical configuration in a mixture.

When a disclosed compound is named or depicted by structure without indicating stereochemistry, it is understood that the name or the structure encompasses one of the possible stereoisomers or geometric isomers free of the others, or a mixture of the encompassed stereoisomers or geometric isomers.

The terms “subject” and “patient” may be used interchangeably, and means a mammal in need of treatment, e.g., companion animals (e.g., dogs, cats, and the like), farm animals (e.g., cows, pigs, horses, sheep, goats and the like) and laboratory animals (e.g., rats, mice, guinea pigs and the like). Typically, the subject is a human in need of treatment.

As used herein, the terms “treatment,” “treat,” and “treating” refer to reversing, alleviating, delaying the onset of, or inhibiting the progress of a disease or disorder, or one or more symptoms thereof, as described herein. In some aspects, treatment may be administered after one or more symptoms have developed, i.e., therapeutic treatment. In other aspects, treatment may be administered in the absence of symptoms. For example, treatment may be administered to a susceptible individual prior to the onset of symptoms (e.g., in light of a history of symptoms and/or in light of exposure to a particular organism, or other susceptibility factors), i.e., prophylactic treatment. Treatment may also be continued after symptoms have resolved, for example to delay their recurrence.

The term “pharmaceutically acceptable carrier” refers to a non-toxic carrier, adjuvant, or vehicle that does not destroy the pharmacological activity of the compound with which it is formulated. Pharmaceutically acceptable carriers, adjuvants or vehicles that may be used in the compositions described herein include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

For use in medicines, the salts of the compounds described herein refer to non-toxic “pharmaceutically acceptable salts.” Pharmaceutically acceptable salt forms include pharmaceutically acceptable acidic/anionic or basic/cationic salts. Suitable pharmaceutically acceptable acid addition salts of the compounds described herein include e.g., salts of inorganic acids (such as hydrochloric acid, hydrobromic, phosphoric, nitric, and sulfuric acids) and of organic acids (such as, acetic acid, benzenesulfonic, benzoic, methanesulfonic, and p-toluenesulfonic acids). Compounds of the present teachings with acidic groups such as carboxylic acids can form pharmaceutically acceptable salts with pharmaceutically acceptable base(s). Suitable pharmaceutically acceptable basic salts include e.g., ammonium salts, alkali metal salts (such as sodium and potassium salts) and alkaline earth metal salts (such as magnesium and calcium salts). Compounds with a quaternary ammonium group also contain a counteranion such as chloride, bromide, iodide, acetate, perchlorate and the like. Other examples of such salts include hydrochlorides, hydrobromides, sulfates, methanesulfonates, nitrates, benzoates and salts with amino acids such as glutamic acid.

The term “effective amount” or “therapeutically effective amount” refers to an amount of a compound described herein that is sufficient to achieve the desired therapeutic effect (such as treatment of a condition recited herein) under the conditions of administration.

### 3. Compounds of the Invention:

In certain embodiments, the STAT3 and/or STAT6 binding moiety is a compound of Formula (I′):

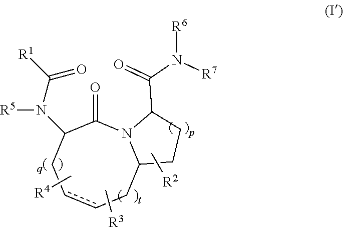

or a pharmaceutically acceptable salt thereof, wherein:
q is 0 or 1 and t is 0, 1, or 2, provided that at least one of q or t is 1;
p is 1 or 2;
the dotted line represents a single or double bond;
R¹ is selected from an 8- to 10-membered fused bicyclic heteroaryl substituted with —CR^{1a}R^{2a}P(O)OR^{1b}OR^{2b} or —CR^{1a}R^{2a}P(O)[OR^{1b}][NH(AA)C(O)OR^{T}], an 8- to 10-membered fused bicyclic heterocyclyl substituted with —CR^{1a}R^{2a}P(O)OR^{1b}OR^{2b} or —CR^{1a}R^{2a}P(O)[OR^{1b}][NH(AA)C(O)OR^{T}], an aryl substituted with CR^{1a}R^{2a}P(O)OR^{1b}OR^{2b} or —CR^{1a}R^{2a}P(O)[OR^{1b}][NH(AA)C(O)OR^{T}], a —(C₁-C₄)alkyl(aryl) wherein said aryl portion of —(C₁-C₄)alkyl(aryl) is substituted with —CR^{1a}R^{2a}P(O)OR^{1b}OR^{2b} or —CR^{1a}R^{2a}P(O)[OR^{1b}][NH(AA)C(O)OR^{T}], and a —(C₂-C₄)alkenyl(aryl) wherein said aryl portion of —(C₂-C₄)alkenyl(aryl) is substituted with —CR^{1a}R^{2a}P(O)OR^{1b}OR^{2b} or —CR^{1a}R^{2a}P(O)[OR^{1b}][NH(AA)C(O)OR^{T}];
R^{1a} and R^{2a} are each absent or are independently selected from hydrogen, cyano, (C₁-C₄)alkyl, hydroxy(C₁-C₄)alkyl and fluoro; or R^{1a} and R^{2a} taken together with the carbon they are attached form oxo;
R^{1b} and R^{2b} are each absent or are independently selected from hydrogen, (C₁-C₄)alkyl, halo(C₁-C₄)alkyl, —[(C₁-C₄)alkyl]-OC(O)—[(C₁-C₄)alkyl], —[(C₁-C₄)alkyl]-C(O)O—[(C₁-C₄)alkyl], —[(C₁-C₄)alkyl]-O—[(C₁-C₂₀)alkyl], —[(C₁-C₄)alkyl]-OC(O)—[halo(C₁-C₄)alkyl], [(C₁-C₄)alkyl]-OC(O)O—[5- to 7-membered heterocyclyl], [(C₁-C₄)alkyl]-OC(O)—[5- to 7-membered heterocyclyl], —[(C₁-C₄)alkyl]-OC(O)—[(C₁-C₄)alkyl]-OH, —[(C₁-C₄)alkyl]-OC(O)—[(C₁-C₄)alkyl]-O—[(C₁-C₄)alkyl], —[(C₁-C₄)alkyl]-OC(O)O—[(C₁-C₄)alkyl], —[(C₁-C₄)alkyl]-OC(O)O—[halo(C₁-C₄)alkyl], —[(C₁-C₄)alkyl]-OC(O)O—[(C₁-C₄)alkyl]-OH, —[(C₁-C₄)alkyl]-OC(O)O—[(C₁-C₄)alkyl]-O—[(C₁-C₄)alkyl], —[(C₁-C₄)alkyl]-SC(O)—[(C₁-C₄)alkyl], —[(C₁-C₄)alkyl]-SC(O)—[halo(C₁-C₄)alkyl], —[(C₁-C₄)alkyl]-SC(O)—[(C₁-C₄)alkyl]-OH, —[(C₁-C₄)alkyl]-SC(O)—[(C₁-C₄)alkyl]-O—[(C₁-C₄)alkyl], —[(C₁-C₄)alkyl]-OC(O)NH(C₁-C₄)alkyl], —[(C₁-C₄)alkyl]-OC(O)N[(C₁-C₄)alkyl]2, 5- to 6-membered heteroaryl, and aryl, wherein said 5- to 6-membered heteroaryl and aryl are each optionally and independently substituted with, as valency permits, 1 to 2 groups selected from halo, cyano, and (C₁-C₄)alkyl and wherein said 5- to 7-membered heterocyclyl of [(C₁-C₄)alkyl]-OC(O)O—[5- to 7-membered heterocyclyl] and [(C₁-C₄)alkyl]-OC(O)—[5- to 7-membered heterocyclyl] are each optionally and independently substituted with, as valency permits 1 to 2 groups selected from C(O)OR^{h};
R² is selected from hydrogen, halo, (C₁-C₄)alkyl, halo(C₁-C₄)alkyl, (C₁-C₄)alkoxy, halo(C₁-C₄)alkoxy, hydroxy(C₁-C₄)alkyl, cyano, and hydroxyl;
R³ and R⁴ are each independently selected from hydrogen, halo, (C₁-C₄)alkyl, halo(C₁-C₄)alkyl, hydroxy(C₁-C₄)alkyl, —(C₁-C₄)alkylphenyl, (C₁-C₄)alkoxy, halo(C₁-C₄)alkoxy, —(C₁-C₄)alkyl(C₁-C₄)alkoxy, hydroxyl, cyano, —NR^{a}R^{b}, phenyl, (C₃-C₆)cycloalkyl, 5- to 6-membered heteroaryl, and 4- to 6-membered heterocyclyl, wherein said phenyl, (C₃-C₆)cycloalkyl, 5- to 6-membered heteroaryl, and 4- to 6-membered heterocyclyl are each optionally substituted with, as valency permits, 1 to 3 groups selected from R^{S};
or R³ and R⁴ are taken together on the same carbon atom to form a (C₃-C₆)cycloalkyl or a 4- to 6-membered heterocyclyl each optionally substituted with, as valency permits, 1 to 3 groups selected from halo, (C₁-C₄)alkyl, halo(C₁-C₄)alkyl, (C₁-C₄)alkoxy, and halo(C₁-C₄)alkoxy;
R⁵ and R⁶ are each independently selected from hydrogen and (C₁-C₄)alkyl;
R⁷ is selected from (C₁-C₄)alkyl, phenyl, 4- to 9-membered monocyclic or bicyclic heterocyclyl, and 5- to 10-membered monocyclic or bicyclic heteroaryl, wherein said (C₁-C₄)alkyl is optionally substituted with, as valency permits, 1 to 3 groups selected from R^{Y} and said phenyl, 4- to 9-membered monocyclic or bicyclic heterocyclyl, and 5- to 10-membered monocyclic or bicyclic heteroaryl are each optionally substituted with, as valency permits, 1 to 3 groups selected from R^{Z}; or
R⁶ and R⁷ together with the nitrogen atom to which they are attached form a 4- to 14-membered monocyclic or bicyclic heterocyclyl or a 5- to 12-membered monocyclic or bicyclic heteroaryl, each of which being optionally substituted with, as valency permits, 1 to 3 groups selected from R^{Q};
AA is the residue of an alpha or beta natural or non-natural amino acid;
R^{T} is selected from (C₁-C₄)alkyl, benzyl, and phenyl, wherein said phenyl is optionally substituted with 1 or 2 groups selected from halo, (C₁-C₄)alkyl, and halo(C₁-C₄)alkyl;
R^{Q} is selected from halo, (C₂-C₄)alkenyl, (C₁-C₄)alkyl, (C₁-C₄)alkoxy, halo(C₁-C₄)alkoxy, cyano, phenyl, hydroxyl, 4- to 6-membered heterocyclyl, 5- to 10-membered monocyclic or bicyclic heteroaryl, (C₃-C₆)cycloalkyl, oxo, imino, —O(phenyl), —C(O)R^{g}, —C(O)OR^{e}, —NHC(O)R^{e}, —C(O)NR^{c}R^{d}, —NR^{a}R^{b}, —S(O)R^{e}R^{f}, —S(O)₂R^{f}, —S(O)═NH(C₁-C₄)alkyl, —S(O)NR^{e}R^{f}, and —S(O)₂NR^{e}R^{f}, wherein said (C₂-C₄)alkenyl and (C₁-C₄)alkyl are each optionally and independently substituted with, as valency permits, 1 to 3 groups selected from R^{M}, and wherein said phenyl, 5- to 10-membered monocyclic or bicyclic heteroaryl, (C₃-C₆)cycloalkyl, and 4- to 6-membered heterocyclyl are each optionally and independently substituted with, as valency permits, 1 to 3 groups selected from R^{F};
R^{Y} is selected from halo, (C₁-C₄)alkoxy, halo(C₁-C₄)alkoxy, cyano, —C(O)R^{g}, —C(O)OR^{e}, —NHC(O)R^{e}, —NR^{a}R^{b}, —S(O)R^{e}R^{f}, —S(O)₂R^{f}, —S(O)NR^{e}R^{f}, —S(O)═NH(C₁-C₄)alkyl, —S(O)₂NR^{e}R^{f}, hydroxyl, phenyl, 4- to 6-membered heterocyclyl, and 5- to 10-membered monocyclic or bicyclic heteroaryl, wherein said phenyl, 4- to 6-membered heterocyclyl, and 5- to 10-membered monocyclic or bicyclic heteroaryl are each optionally substituted with, as valency permits, 1 to 3 groups selected from R^{X};
R^{J} and R^{M} are each independently selected from halo, (C₁-C₄)alkoxy, halo(C₁-C₄)alkoxy, cyano, —C(O)R^{g}, —C(O)OR^{e}, —NHC(O)R^{e}, —C(O)NR^{c}R^{d}, —NR^{a}R^{b}, —S(O)R^{e}R^{f}, —S(O)₂R^{f}, —S(O)NR^{e}R^{f}, —S(O)═NH(C₁-C₄)alkyl, —S(O)₂NR^{e}R^{f}, hydroxyl, phenyl, 4- to 6-membered heterocyclyl, and 5- to 10-membered monocyclic or bicyclic heteroaryl, wherein said phenyl, 4- to 6-membered heterocyclyl, and 5- to 10-membered monocyclic or bicyclic heteroaryl are each optionally substituted with, as valency permits, 1 to 3 groups selected from R^{X};
R^{F}, R^{S}, R^{X}, and R^{Z} are each independently selected from halo, cyano, (C₁-C₄)alkyl, halo(C₁-C₄)alkyl, —(C₁-C₄)alkyl(C₁-C₄)alkoxy, hydroxy(C₁-C₄)alkyl, —(C₁-C₄)alkylphenyl, (C₂-C₄)alkenyl, halo(C₂-C₄)alkenyl, (C₂-C₄)alkynyl, halo(C₂-C₄)alkynyl, (C₁-C₄)alkoxy, halo(C₁-C₄)alkoxy, hydroxyl, oxo, imino, phenyl, —S(O)R^{e}R^{f}, —S(O)₂R^{f}, —S(O)═NH(C₁-C₄)alkyl, —S(O)NR^{e}R^{f}, and —S(O)₂NR^{e}R^{f}, —C(O)OR^{e}, —NR^{c}C(O)R^{e}, —C(O)R^{g}, —C(O)NR^{c}R^{d}, and —NR^{a}R^{b}, wherein said phenyl and said phenyl for the group —(C₁-C₄)alkylphenyl are each optionally and independently substituted with, as valency permits 1 to 3 groups selected from halo, cyano, (C₁-C₁₀)alkyl, (C₂-C₁₀)alkenyl, (C₂-C₁₀)alkynyl, halo(C₁-C₁₀)alkyl, (C₁-C₁₀)alkoxy, and halo(C₁-C₁₀)alkoxy, wherein said (C₁-C₁₀)alkyl, (C₂-C₁₀)alkenyl and (C₂-C₁₀)alkynyl are each optionally substituted with, as valency permits a 5- to 10-membered monocyclic or bicyclic heteroaryl or a 4- to 10-membered monocyclic or bicyclic heterocyclyl each of said 5- to 10-membered monocyclic and bicyclic heteroaryl or a 4- to 10-membered monocyclic or bicyclic heterocyclyl being optionally substituted with oxo or a 5- to 7-membered heterocyclyl that is optionally substituted with 1 to 2 oxo; and
R^{a}, R^{b}, R^{c}, R^{d}, R^{e}, R^{f}, R^{g}, and R^{h} are each independently selected from, as valency permits, hydrogen, (C₁-C₄)alkyl, phenyl, (C₃-C₆)cycloalkyl, 4- to 6-membered heterocyclyl and 5- to 6-membered heteroaryl, wherein said (C₁-C₄)alkyl is optionally substituted with, as valency permits, 1 to 3 groups selected from R^{J}, and said phenyl, (C₃-C₆)cycloalkyl, 4- to 6-membered heterocyclyl, and 5- to 6-membered heteroaryl are each independently optionally substituted with, as valency permits, 1 to 3 groups selected from halo, cyano, (C₁-C₄)alkyl, halo(C₁-C₄)alkyl, (C₁-C₄)alkoxy, halo(C₁-C₄)alkoxy, hydroxyl, phenyl, and benzyl.

In certain embodiments from the present invention, the L and DM moieties are described herein. In certain embodiments, the L and DM moieties are provided in WO 2024/064080, which is incorporated by reference in its entirety.

In some embodiments, DM is an E3 ligase ligand well known to one of ordinary skill in the art including those described in M. Toure, C. M. Crews, Angew. Chem. Int. Ed., 2016, 55, 1966, T. Uehara et al. Nature Chemical Biology, 2017, 13, 675, WO 2017/176708, US 2017/0281784, WO 2017/161119, WO 2017/176957, WO 2017/176958, WO 2015/160845, US 2015/0291562, WO 2016/197032, WO 2016/105518, US 2018/0009779, WO 2017/007612, 2018/0134684, WO 2013/106643, US 2014/0356322, WO 2002/020740, US 2002/0068063, WO 2012/078559, US 2014/0302523, WO 2012/003281, US 2013/0190340, US 2016/0022642, WO 2014/063061, US 2015/0274738, WO 2016/118666, US 2016/0214972, WO 2016/149668, US 2016/0272639, WO 2016/169989, US 2018/0118733, WO 2016/197114, US 2018/0147202, WO 2017/011371, US 2017/0008904, WO 2017/011590, US 2017/0037004, WO 2017/079267, US 2017/0121321, WO 2017/117473, WO 2017/117474, WO 2013/106646, WO 2014/108452, WO 2017/197036, US 2019/0076540, WO 2017/197046, US 2019/0076542, WO 2017/197051, US 2019/0076539, WO 2017/197055, US 2019/0076541, and WO 2017/197056, the entirety of each of which is herein incorporated by reference.

In certain embodiments, L is attached to a modifiable carbon, oxygen, or nitro substitution or replacement group on one or both of DM and TBM.

In certain embodiments, the DM is a cereblon E3 ubiquitin ligase binding moiety (hereinafter also referred to as LBM).

In some embodiments, LBM is selected from the group consisting of:

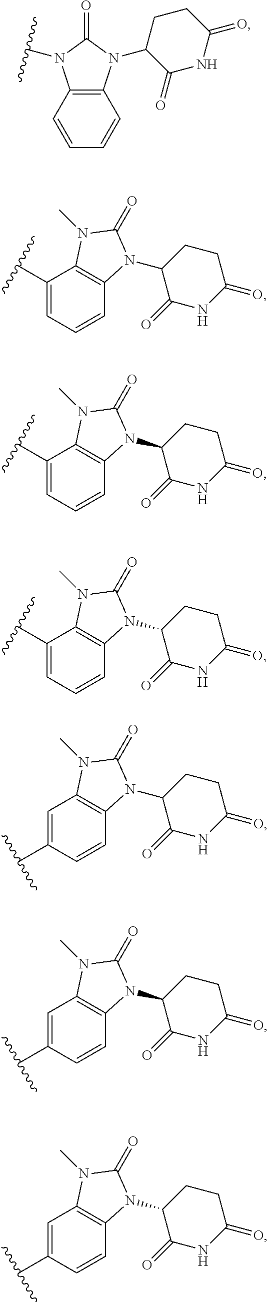

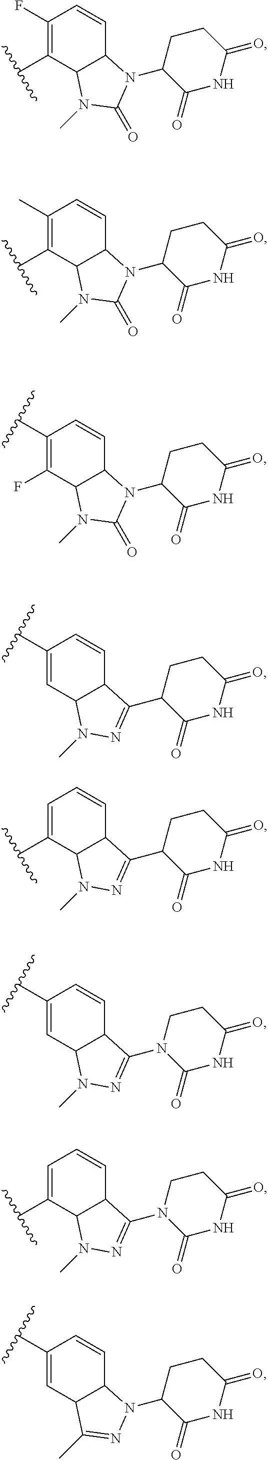

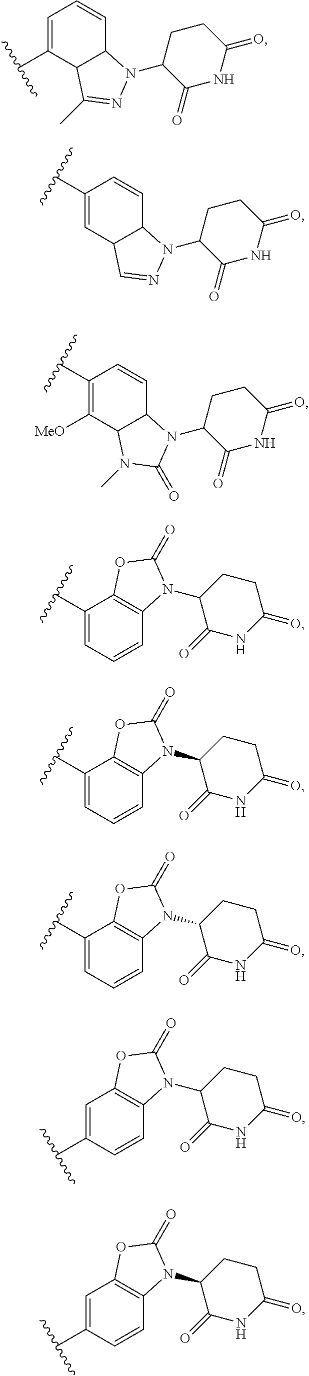

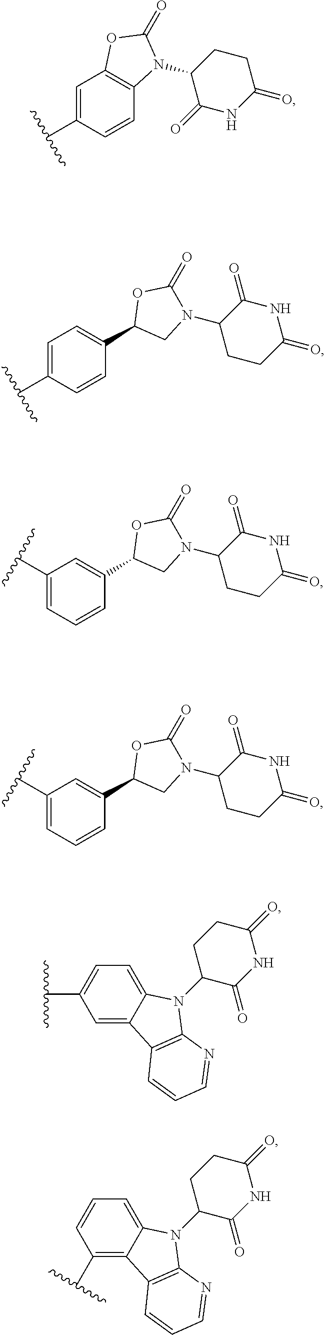 [SMILES: *c1ccc2c(c1)oc(=O)n2[C@@H]1CCC(=O)NC1=O ; *c1ccc([C@@H]2CN(C3CCC(=O)NC3=O)C(=O)O2)cc1 ; *c1cccc([C@H]2CN(C3CCC(=O)NC3=O)C(=O)O2)c1 ; *c1cccc([C@@H]2CN(C3CCC(=O)NC3=O)C(=O)O2)c1 ; *c1ccc2c(c1)c1cccnc1n2C1CCC(=O)NC1=O ; *c1cccc2c1c1cccnc1n2C1CCC(=O)NC1=O]

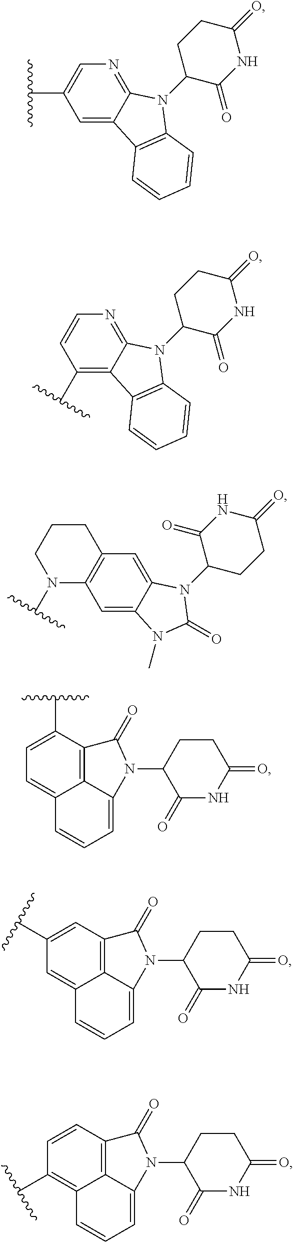

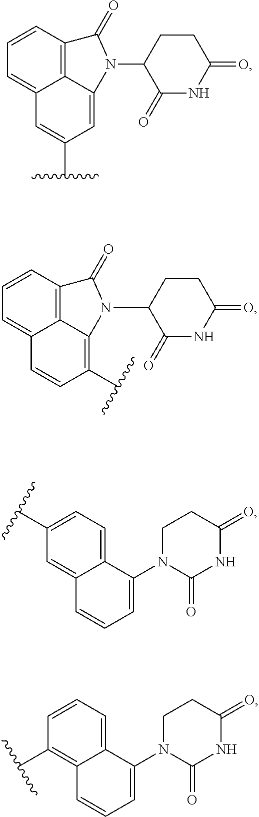 [SMILES: *c1cc2c3c(cccc3c1)C(=O)N2C1CCC(=O)NC1=O ; *c1ccc2cccc3c2c1N(C1CCC(=O)NC1=O)C3=O ; *c1ccc2c(N3CCC(=O)NC3=O)cccc2c1 ; *c1cccc2c(N3CCC(=O)NC3=O)cccc12]

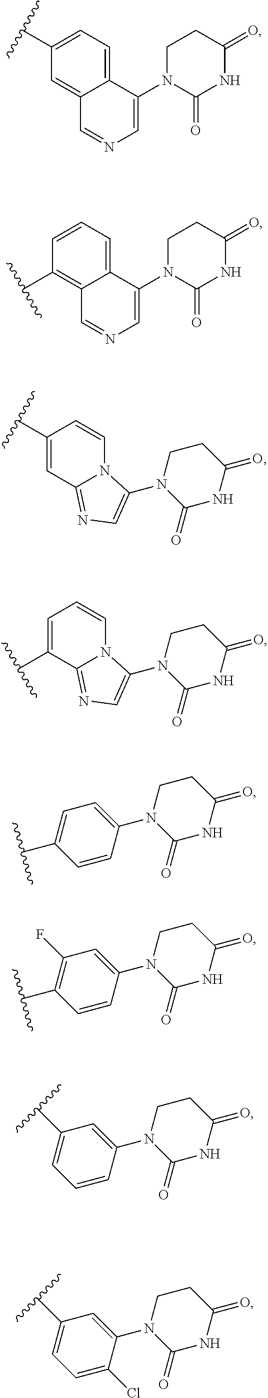

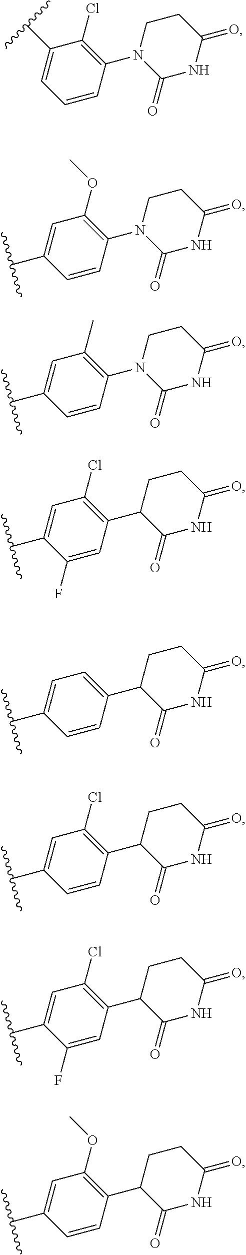

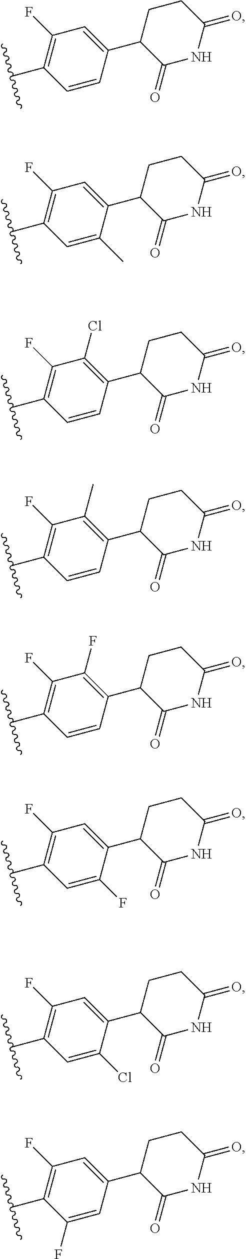

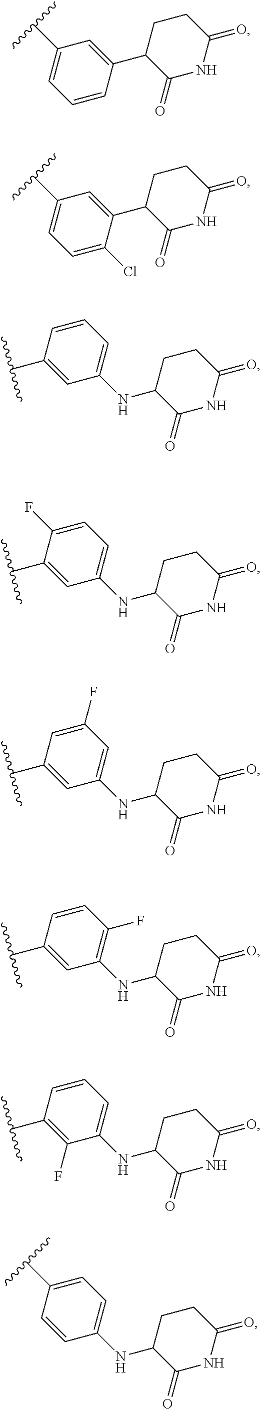

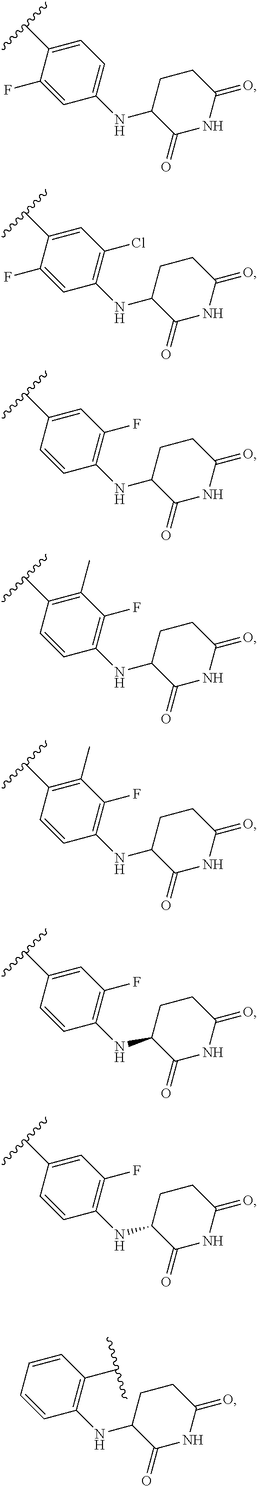

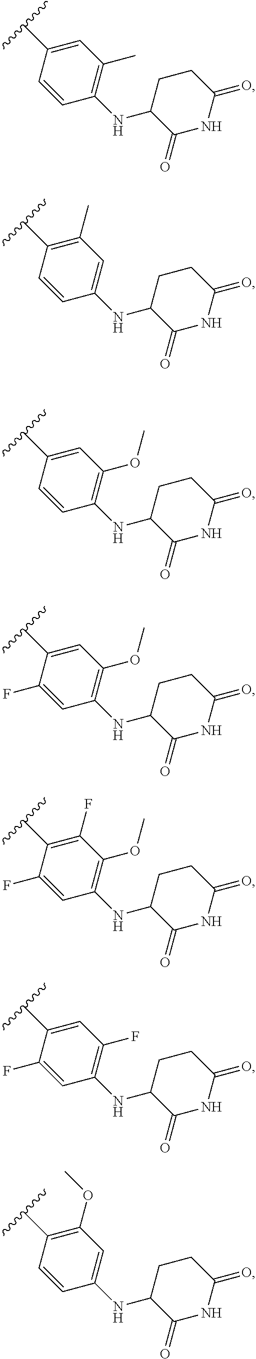

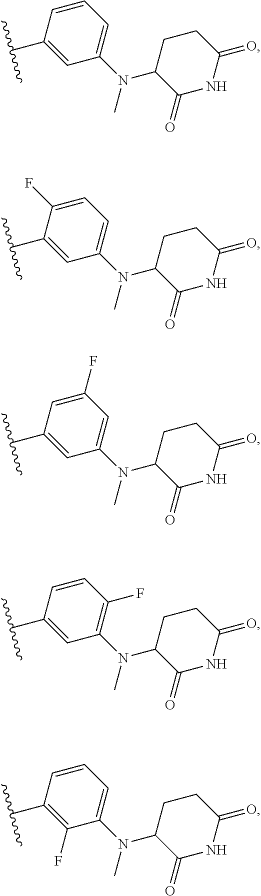 [SMILES: *c1cccc(N(C)C2CCC(=O)NC2=O)c1 ; *c1cc(N(C)C2CCC(=O)NC2=O)ccc1F ; *c1cc(F)cc(N(C)C2CCC(=O)NC2=O)c1 ; *c1ccc(F)c(N(C)C2CCC(=O)NC2=O)c1 ; *c1cccc(N(C)C2CCC(=O)NC2=O)c1F]

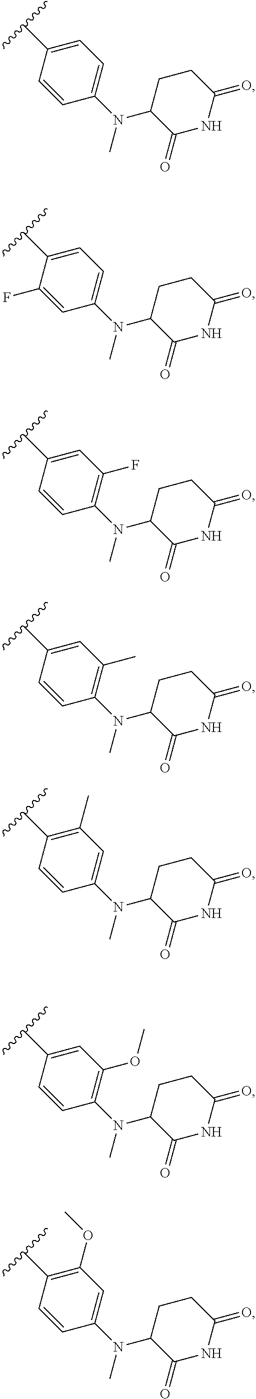

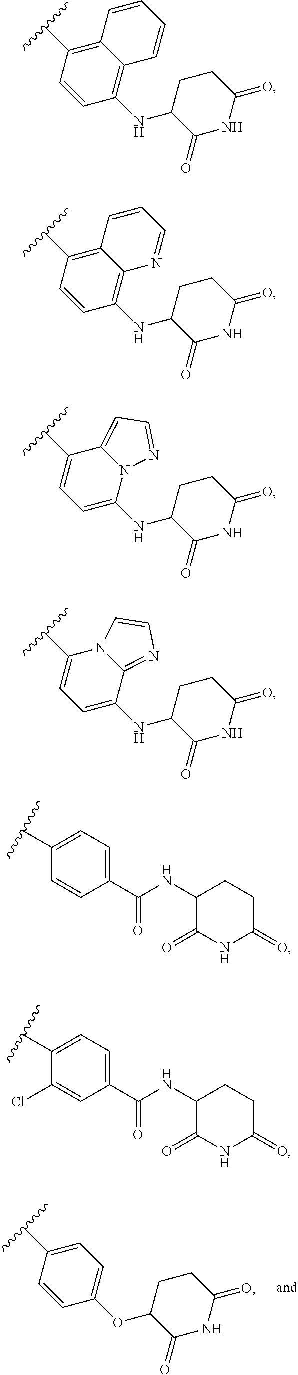

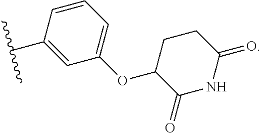 [SMILES: *c1cccc(OC2CCC(=O)NC2=O)c1]

In some embodiments, LBM is E3 Ubiquitin ligase binding moiety recited in Varfolomeev, E. et al., IAP Antagonists Induce Autoubiquitination of c-IAPs, NF-κB activation, and TNFα-Dependent Apoptosis, Cell, 2007, 131(4): 669-81, such as, for example:

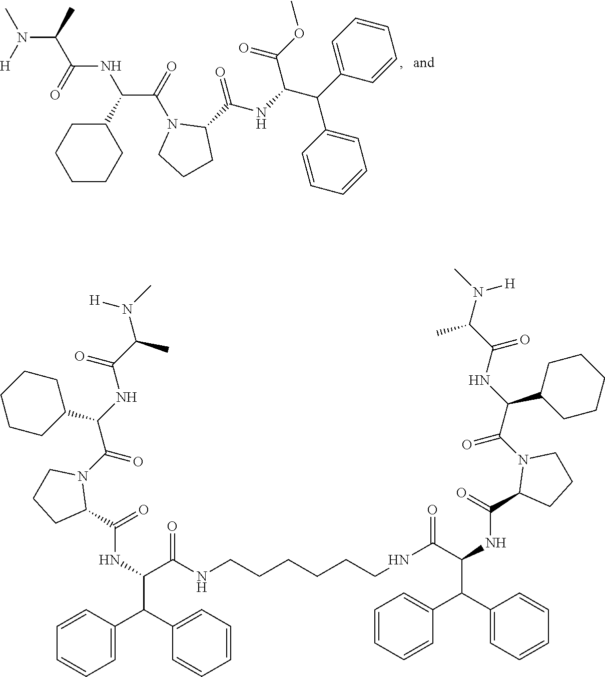

wherein L is attached to a modifiable carbon, oxygen, nitrogen or sulfur atom.

In certain embodiments, LBM is selected from the group consisting of

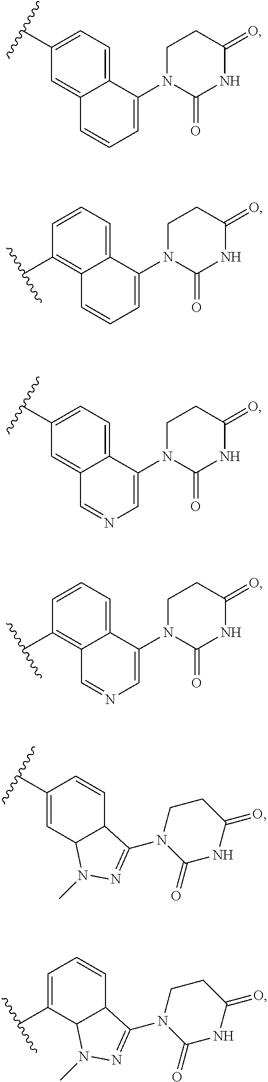 [SMILES: *c1ccc2c(N3CCC(=O)NC3=O)cccc2c1 ; *c1cccc2c(N3CCC(=O)NC3=O)cccc12 ; *c1ccc2c(N3CCC(=O)NC3=O)cncc2c1 ; *c1cccc2c(N3CCC(=O)NC3=O)cncc12 ; *C1=CC2C(C=C1)C(N1CCC(=O)NC1=O)=NN2C ; *C1=CC=CC2C(N3CCC(=O)NC3=O)=NN(C)C12]

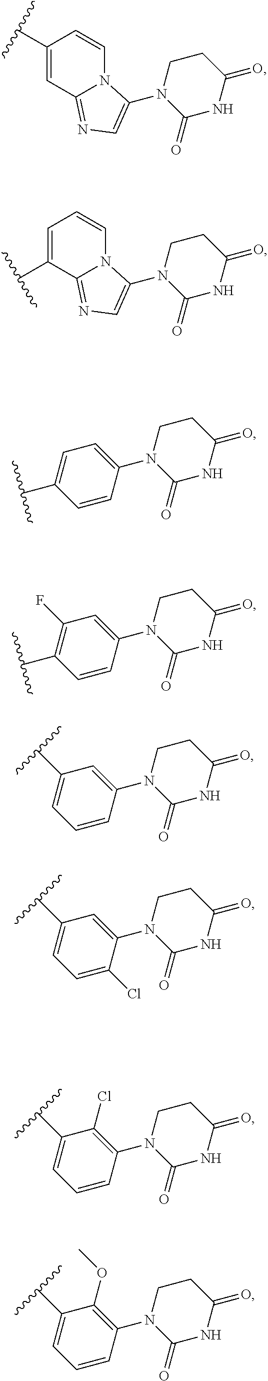

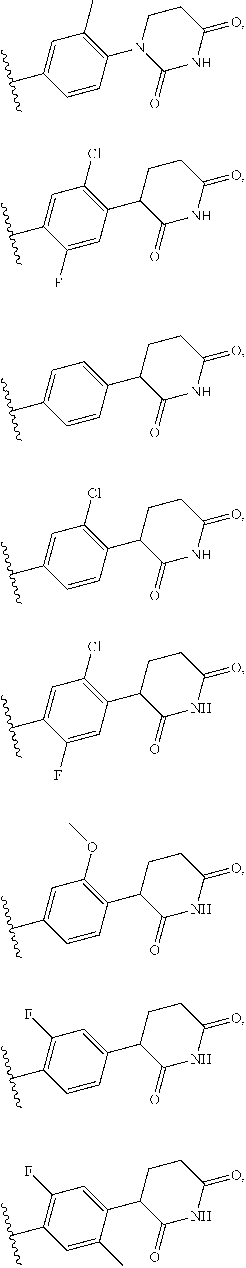

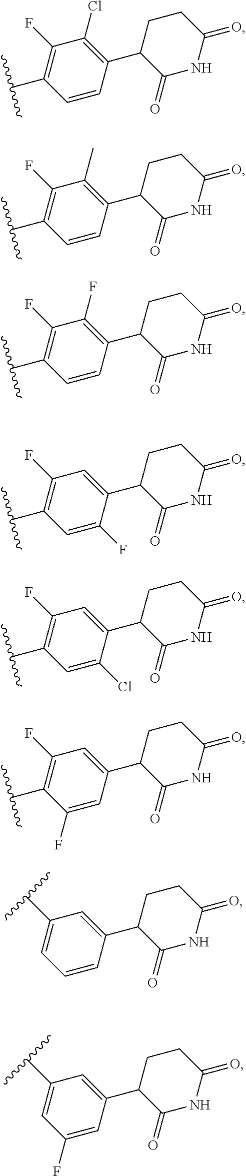

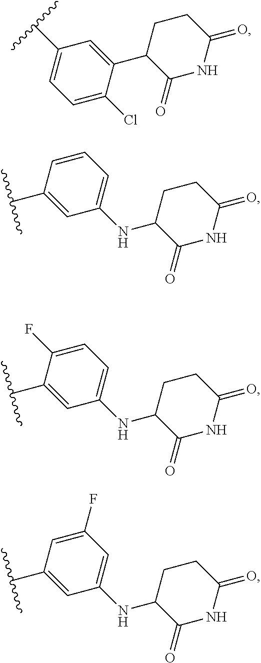 [SMILES: *c1ccc(Cl)c(C2CCC(=O)NC2=O)c1 ; *c1cccc(NC2CCC(=O)NC2=O)c1 ; *c1cc(NC2CCC(=O)NC2=O)ccc1F ; *c1cc(F)cc(NC2CCC(=O)NC2=O)c1]

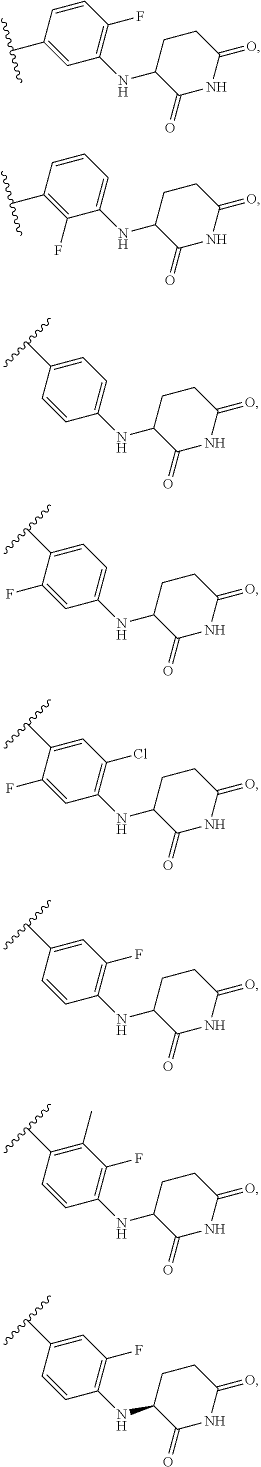

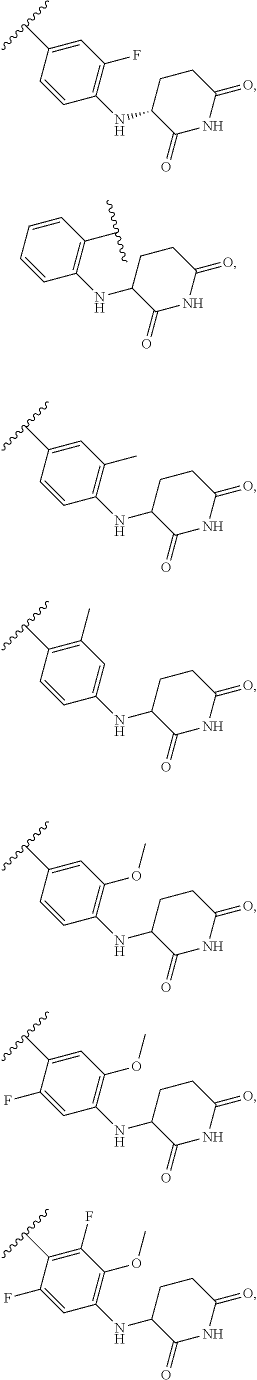

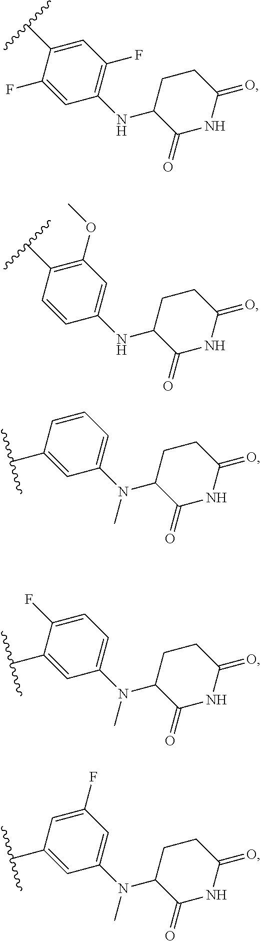 [SMILES: *c1cc(F)c(NC2CCC(=O)NC2=O)cc1F ; *c1ccc(NC2CCC(=O)NC2=O)cc1OC ; *c1cccc(N(C)C2CCC(=O)NC2=O)c1 ; *c1cc(N(C)C2CCC(=O)NC2=O)ccc1F ; *c1cc(F)cc(N(C)C2CCC(=O)NC2=O)c1]

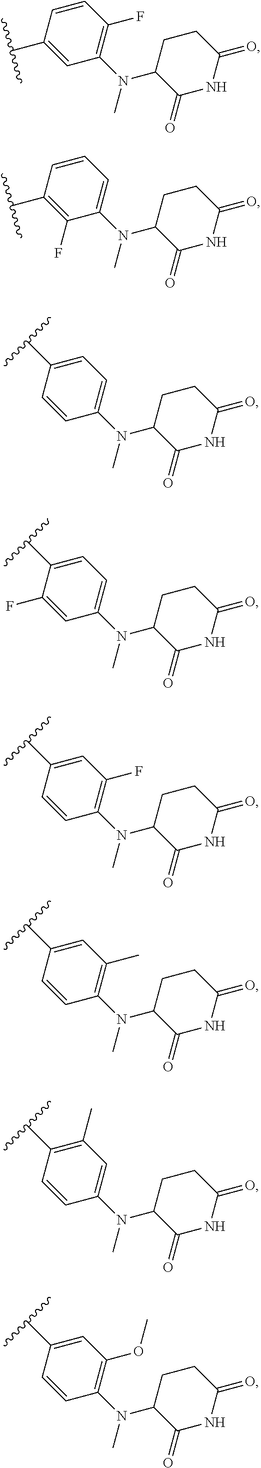

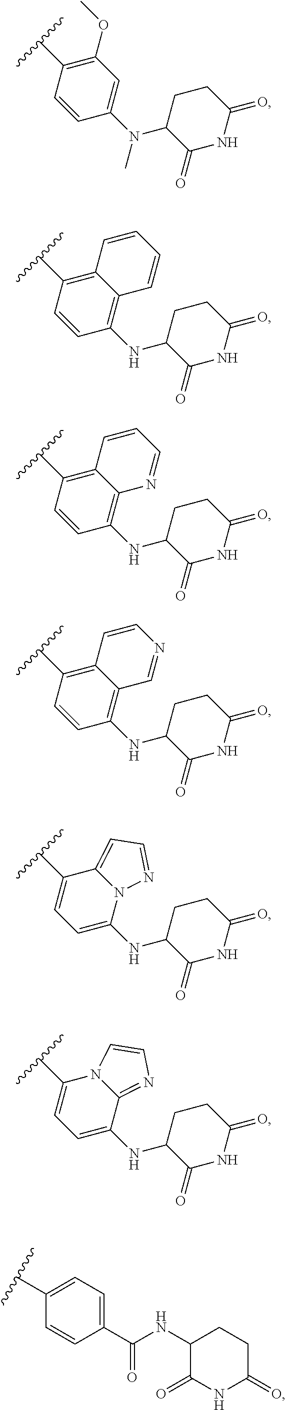

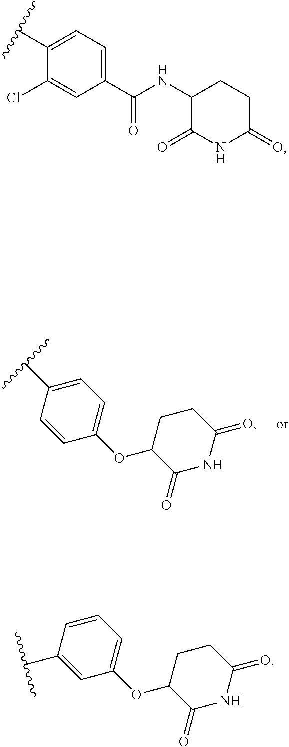 [SMILES: *c1ccc(C(=O)NC2CCC(=O)NC2=O)cc1Cl ; *c1ccc(OC2CCC(=O)NC2=O)cc1 ; *c1cccc(OC2CCC(=O)NC2=O)c1]

In certain embodiments, the LBM is selected from the group consisting of

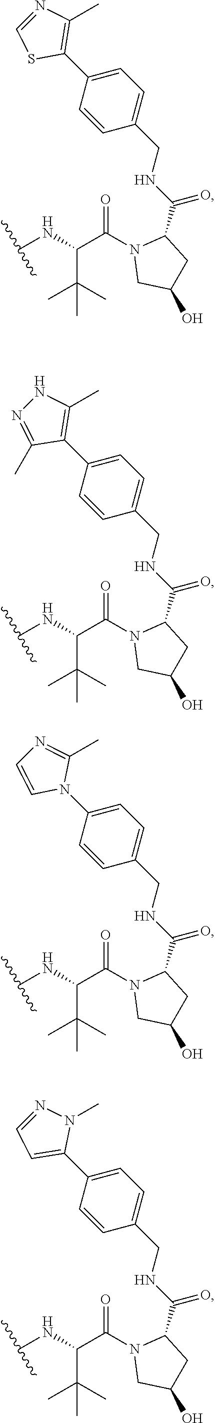

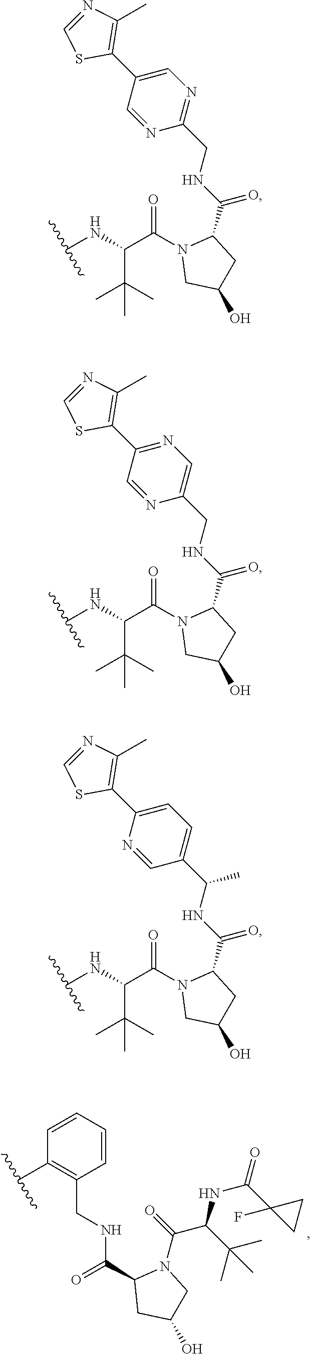 [SMILES: *N[C@H](C(=O)N1C[C@H](O)C[C@H]1C(=O)NCc1ncc(-c2scnc2C)cn1)C(C)(C)C ; *N[C@H](C(=O)N1C[C@H](O)C[C@H]1C(=O)NCc1cnc(-c2scnc2C)cn1)C(C)(C)C ; *N[C@H](C(=O)N1C[C@H](O)C[C@H]1C(=O)N[C@@H](C)c1ccc(-c2scnc2C)nc1)C(C)(C)C ; *c1ccccc1CNC(=O)[C@@H]1C[C@@H](O)CN1C(=O)[C@@H](NC(=O)C1(F)CC1)C(C)(C)C]

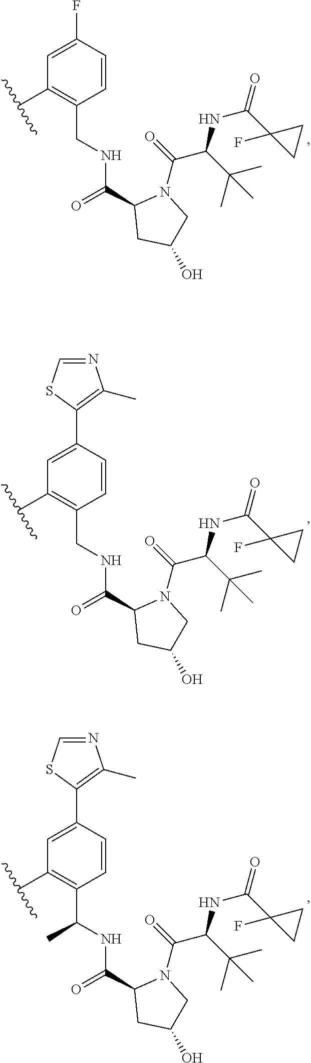 [SMILES: *c1cc(F)ccc1CNC(=O)[C@@H]1C[C@@H](O)CN1C(=O)[C@@H](NC(=O)C1(F)CC1)C(C)(C)C ; *c1cc(-c2scnc2C)ccc1CNC(=O)[C@@H]1C[C@@H](O)CN1C(=O)[C@@H](NC(=O)C1(F)CC1)C(C)(C)C ; *c1cc(-c2scnc2C)ccc1[C@H](C)NC(=O)[C@@H]1C[C@@H](O)CN1C(=O)[C@@H](NC(=O)C1(F)CC1)C(C)(C)C]

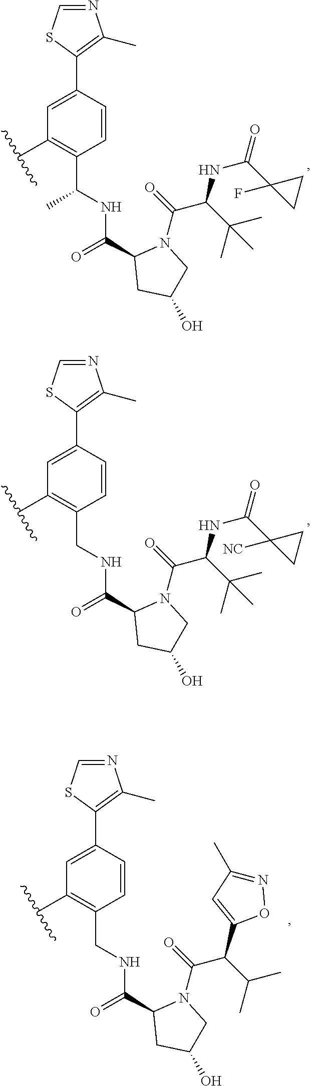 [SMILES: *c1cc(-c2scnc2C)ccc1[C@@H](C)NC(=O)[C@@H]1C[C@@H](O)CN1C(=O)[C@@H](NC(=O)C1(F)CC1)C(C)(C)C ; *c1cc(-c2scnc2C)ccc1CNC(=O)[C@@H]1C[C@@H](O)CN1C(=O)[C@@H](NC(=O)C1(C#N)CC1)C(C)(C)C ; *c1cc(-c2scnc2C)ccc1CNC(=O)[C@@H]1C[C@@H](O)CN1C(=O)[C@@H](c1cc(C)no1)C(C)C]

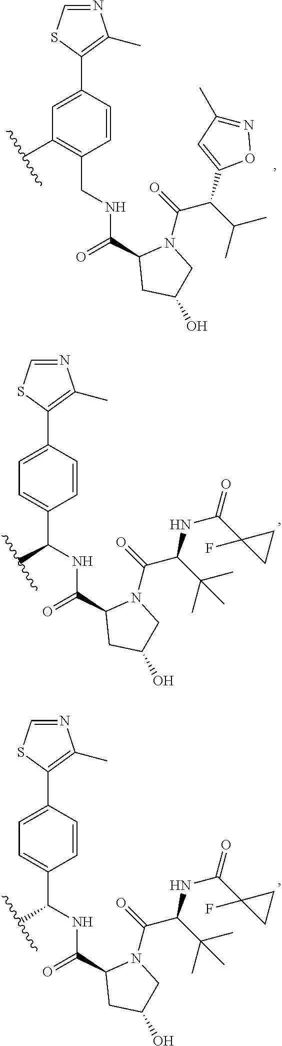 [SMILES: *c1cc(-c2scnc2C)ccc1CNC(=O)[C@@H]1C[C@@H](O)CN1C(=O)[C@H](c1cc(C)no1)C(C)C ; *[C@H](NC(=O)[C@@H]1C[C@@H](O)CN1C(=O)[C@@H](NC(=O)C1(F)CC1)C(C)(C)C)c1ccc(-c2scnc2C)cc1 ; *[C@@H](NC(=O)[C@@H]1C[C@@H](O)CN1C(=O)[C@@H](NC(=O)C1(F)CC1)C(C)(C)C)c1ccc(-c2scnc2C)cc1]

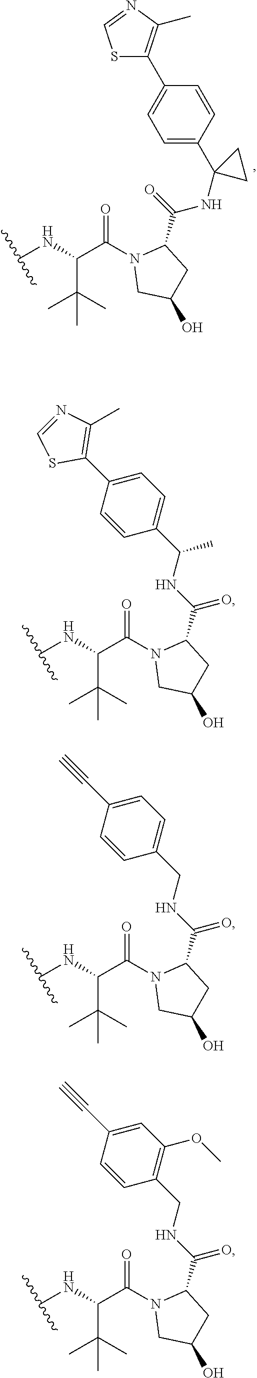 [SMILES: *N[C@H](C(=O)N1C[C@H](O)C[C@H]1C(=O)NC1(c2ccc(-c3scnc3C)cc2)CC1)C(C)(C)C ; *N[C@H](C(=O)N1C[C@H](O)C[C@H]1C(=O)N[C@@H](C)c1ccc(-c2scnc2C)cc1)C(C)(C)C ; *N[C@H](C(=O)N1C[C@H](O)C[C@H]1C(=O)NCc1ccc(C#C)cc1)C(C)(C)C ; *N[C@H](C(=O)N1C[C@H](O)C[C@H]1C(=O)NCc1ccc(C#C)cc1OC)C(C)(C)C]

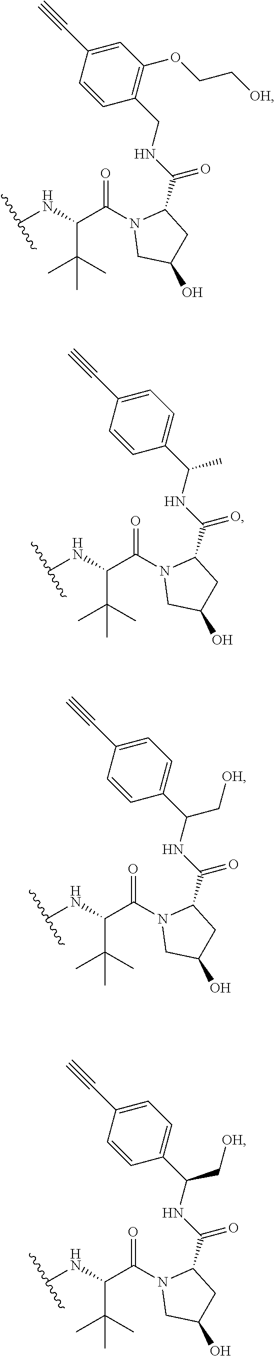 [SMILES: *N[C@H](C(=O)N1C[C@H](O)C[C@H]1C(=O)NCc1ccc(C#C)cc1OCCO)C(C)(C)C ; *N[C@H](C(=O)N1C[C@H](O)C[C@H]1C(=O)N[C@@H](C)c1ccc(C#C)cc1)C(C)(C)C ; *N[C@H](C(=O)N1C[C@H](O)C[C@H]1C(=O)NC(CO)c1ccc(C#C)cc1)C(C)(C)C ; *N[C@H](C(=O)N1C[C@H](O)C[C@H]1C(=O)N[C@H](CO)c1ccc(C#C)cc1)C(C)(C)C]

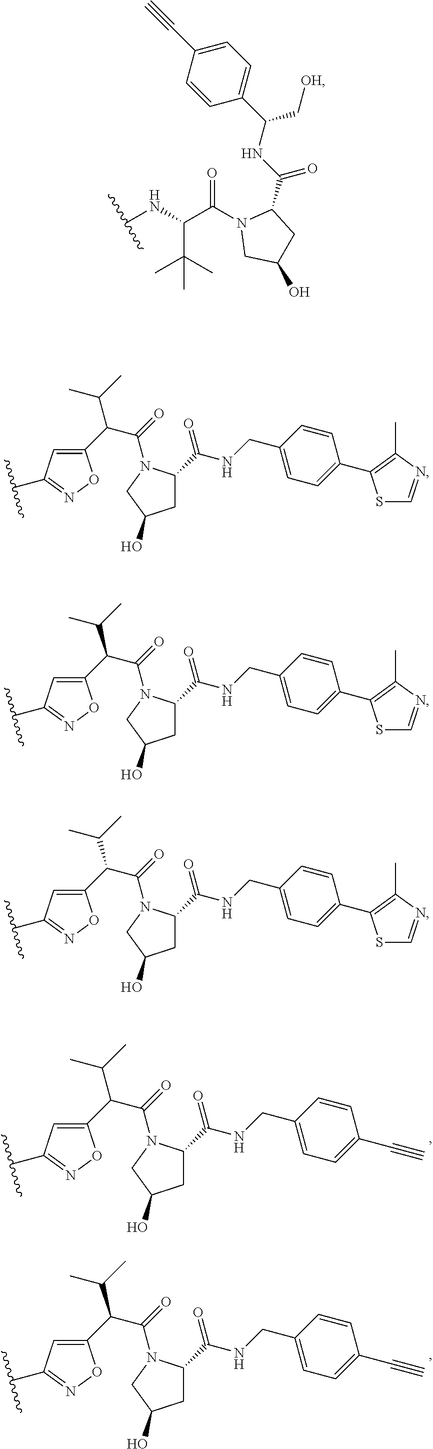

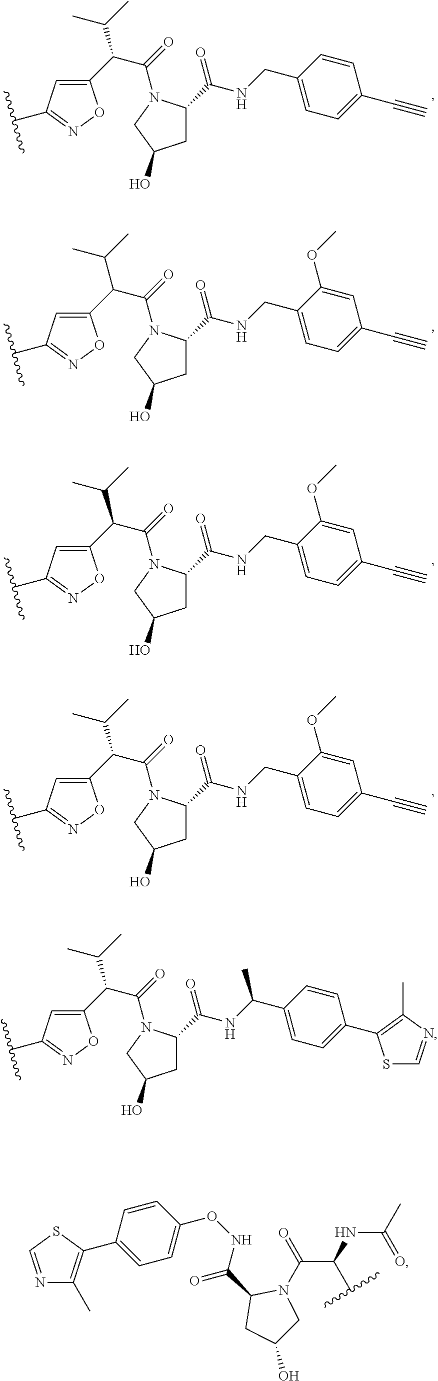 [SMILES: *c1cc([C@@H](C(=O)N2C[C@H](O)C[C@H]2C(=O)NCc2ccc(C#C)cc2)C(C)C)on1 ; *c1cc(C(C(=O)N2C[C@H](O)C[C@H]2C(=O)NCc2ccc(C#C)cc2OC)C(C)C)on1 ; *c1cc([C@H](C(=O)N2C[C@H](O)C[C@H]2C(=O)NCc2ccc(C#C)cc2OC)C(C)C)on1 ; *c1cc([C@@H](C(=O)N2C[C@H](O)C[C@H]2C(=O)NCc2ccc(C#C)cc2OC)C(C)C)on1 ; *c1cc([C@@H](C(=O)N2C[C@H](O)C[C@H]2C(=O)N[C@@H](C)c2ccc(-c3scnc3C)cc2)C(C)C)on1 ; *[C@H](NC(C)=O)C(=O)N1C[C@H](O)C[C@H]1C(=O)NOc1ccc(-c2scnc2C)cc1]

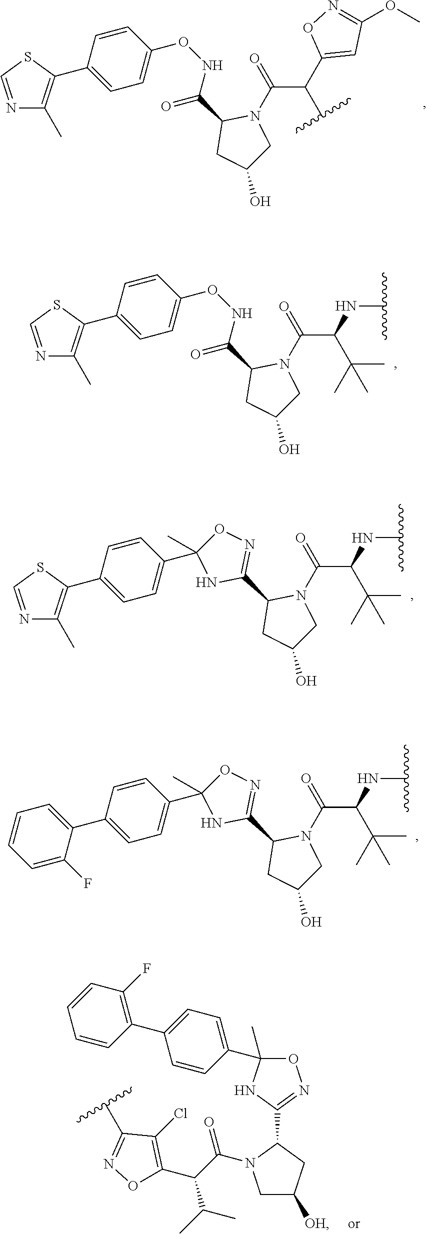 [SMILES: *C(C(=O)N1C[C@H](O)C[C@H]1C(=O)NOc1ccc(-c2scnc2C)cc1)c1cc(OC)no1 ; *N[C@H](C(=O)N1C[C@H](O)C[C@H]1C(=O)NOc1ccc(-c2scnc2C)cc1)C(C)(C)C ; *N[C@H](C(=O)N1C[C@H](O)C[C@H]1C1=NOC(C)(c2ccc(-c3scnc3C)cc2)N1)C(C)(C)C ; *N[C@H](C(=O)N1C[C@H](O)C[C@H]1C1=NOC(C)(c2ccc(-c3ccccc3F)cc2)N1)C(C)(C)C ; *c1noc([C@H](C(=O)N2C[C@H](O)C[C@H]2C2=NOC(C)(c3ccc(-c4ccccc4F)cc3)N2)C(C)C)c1Cl]

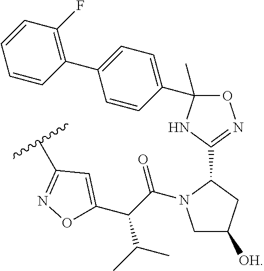 [SMILES: *c1cc([C@H](C(=O)N2C[C@H](O)C[C@H]2C2=NOC(C)(c3ccc(-c4ccccc4F)cc3)N2)C(C)C)on1]

In certain embodiments, LBM is selected from the group consisting of

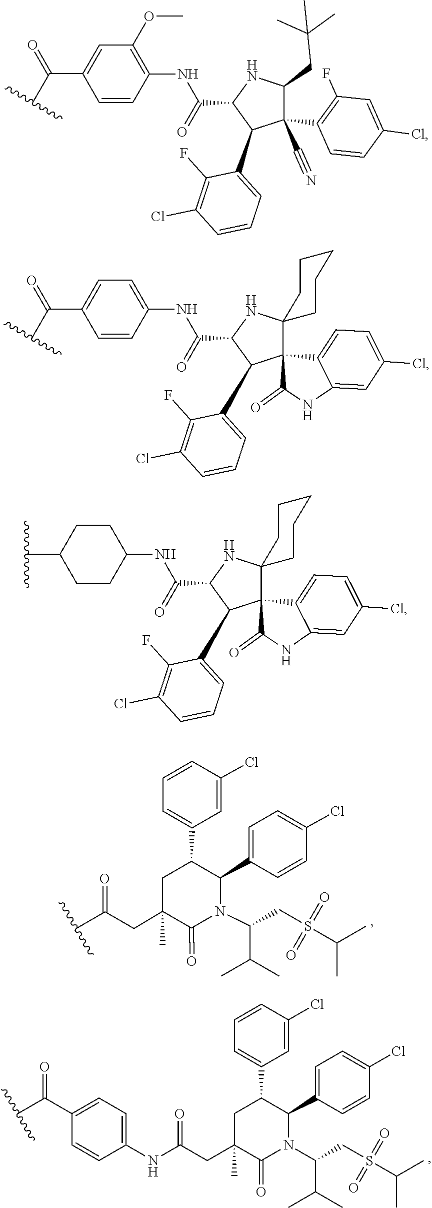

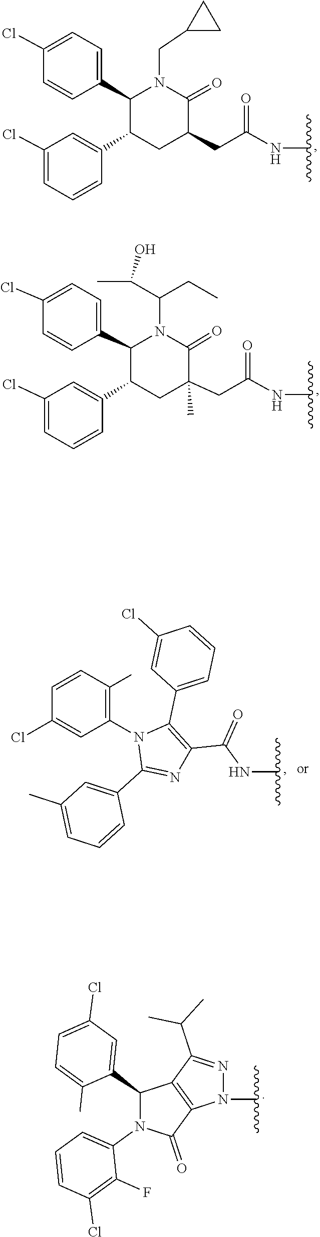 [SMILES: *NC(=O)C[C@H]1C[C@H](c2cccc(Cl)c2)[C@@H](c2ccc(Cl)cc2)N(CC2CC2)C1=O ; *NC(=O)C[C@@]1(C)C[C@H](c2cccc(Cl)c2)[C@@H](c2ccc(Cl)cc2)N(C(CC)[C@H](C)O)C1=O ; *NC(=O)c1nc(-c2cccc(C)c2)n(-c2cc(Cl)ccc2C)c1-c1cccc(Cl)c1 ; *n1nc(C(C)C)c2c1C(=O)N(c1cccc(Cl)c1F)[C@H]2c1cc(Cl)ccc1C]

As described herein, L is a bivalent moiety that connects TBM and DM.

In certain embodiments, L is a bivalent moiety that connects TBM to a lysine mimetic.

In certain embodiments, L is a covalent bond or a bivalent, saturated or partially unsaturated, straight or branched C₁₋₅₀ hydrocarbon chain, wherein 0-6 methylene units of L may optionally be replaced by -Cy-, —CHF—, —CF₂—, —O—, —NR—, —SiR₂—, —Si(OH)R—, —Si(OH)₂—, —P(O)OR—, —P(O)R—, —P(O)NR₂—, —S—, —OC(O)—, —C(O)O—, —C(O)—, —S(O)—, —S(O)₂—, NRS(O)₂—, —S(O)₂NR—, —NRC(O)—, —C(O)NR—, —OC(O)NR—, NRC(O)O—,

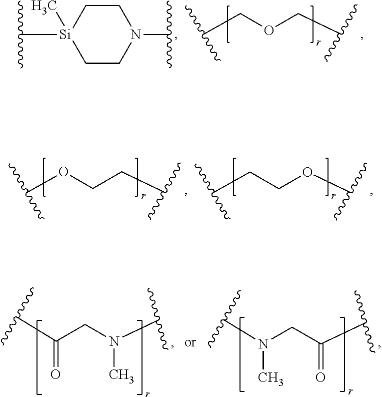

wherein:
each -Cy- is independently an optionally substituted bivalent ring selected from phenylenyl, an 8-10 membered bicyclic arylenyl, a 4-7 membered saturated or partially unsaturated carbocyclylenyl, a 6-11 membered saturated or partially unsaturated spiro carbocyclylenyl, an 8-10 membered bicyclic saturated or partially unsaturated carbocyclylenyl, a 4-7 membered saturated or partially unsaturated heterocyclylenyl having 1-2 heteroatoms independently selected from nitrogen, oxygen, and sulfur, a 6-11 membered saturated or partially unsaturated spiro heterocyclylenyl having 1-2 heteroatoms independently selected from nitrogen, oxygen, and sulfur, an 8-10 membered bicyclic saturated or partially unsaturated heterocyclylenyl having 1-2 heteroatoms independently selected from nitrogen, oxygen, and sulfur, a 5-6 membered heteroarylenyl having 1-4 heteroatoms independently selected from nitrogen, oxygen, and sulfur, and an 8-10 membered bicyclic heteroarylenyl having 1-5 heteroatoms independently selected from nitrogen, oxygen, and sulfur,
each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or: two R groups on the same nitrogen are optionally taken together with their intervening atoms to forn1 a 4-7 membered saturated, partially unsaturated, or heteroaryl ring having 0-3 heteroatoms, in addition to the nitrogen, independently selected from nitrogen, oxygen, and sulfur, and;
r is 0, 1, 2, 3, 4, 5, 6, 7, 8, 9, or 10.

In some embodiments, each -Cy- is independently an optionally substituted bivalent phenylenyl. In some embodiments, each -Cy- is independently an optionally substituted 8-10 membered bicyclic arylenyl. In some embodiments, each -Cy- is independently an optionally substituted 4-7 membered saturated or partially unsaturated carbocyclylenyl. In some embodiments, each -Cy- is independently an optionally substituted 6-11 membered saturated or partially unsaturated spiro-carbocyclylenyl. In some embodiments, each -Cy- is independently an optionally substituted 8-10 membered bicyclic saturated or partially unsaturated carbocyclylenyl. In some embodiments, each -Cy is independently an optionally substituted 4-7 membered saturated or partially unsaturated heterocyclylenyl having 1-2 heteroatoms independently selected from nitrogen, oxygen, and sulfur. In some embodiments, each -Cy- is independently an optionally substituted 6-11 membered saturated or partially unsaturated spiro-heterocyclylenyl having 1-2 heteroatoms independently selected from nitrogen, oxygen, and sulfur. In some embodiments, each -Cy- is independently an optionally substituted 8-10 membered bicyclic saturated or partially unsaturated heterocyclylenyl having 1-2 heteroatoms independently selected from nitrogen, oxygen, and sulfur. In some embodiments, each -Cy- is independently an optionally substituted 5-6 membered heteroarylenyl having 1-4 heteroatoms independently selected from nitrogen, oxygen, and sulfur. In some embodiments, each -Cy- is independently an optionally substituted 8-10 membered bicyclic heteroarylenyl having 1-5 heteroatoms independently selected from nitrogen, oxygen, and sulfur.

In some embodiments, -Cy- is substituted with C₁0.6 alkyl (e.g., methyl, ethyl, isopropyl). In some embodiments, -Cy- is substituted with oxo. In some embodiments, -Cy- is substituted with halogen. In some embodiments, -Cy- is substituted with fluoro. In some embodiments, -Cy- is substituted with geminal difluoro. In some embodiments, -Cy- is substituted with —OH. In some embodiments, -Cy- is substituted with —NR₂.

In some embodiments, r is 0. In some embodiments, r is 1. In some embodiments, r is 2. In some embodiments, r is 3. In some embodiments, r is 4. In some embodiments, r is 5. In some embodiments, r is 6. In some embodiments, r is 7. In some embodiments, r is 8. In some embodiments, r is 9. In some embodiments, r is 10.

In some embodiments, L is —NR—(C₁₋₁₀ aliphatic)-. In some embodiments, L is —(C₁₋₁₀ aliphatic)-NR—(C₁-10aliphatic)-. In some embodiments, L is —(C₁₋₁₀ aliphatic)-NR—(CH₂CH₂O)₁₋₁₀CH₂CH₂—. In some embodiments, L is -Cy-NR—(C₁₋₁₀ aliphatic)-. In some embodiments, L is -Cy-(C₁₋₁₀ aliphatic)-NR—. In some embodiments, L is -Cy-(C₁₋₁₀ aliphatic)-NR—(C₁₋₁₀ aliphatic)-. In some embodiments, L is —(C₁₋₁₀ aliphatic)-Cy-NR—(C₁₋₁₀ aliphatic)-. In some embodiments, L is —(C₁₋₁₀ aliphatic)-Cy-(C₁₋₁₀ aliphatic)-NR—. In some embodiments, L is —(C₁₋₁₀ aliphatic)-Cy-(C₁₋₁₀ aliphatic)-NR—(C₁₋₁₀ aliphatic)-. In some embodiments, L is -Cy-(C₁₋₁₀ aliphatic)-Cy-NR—. In some embodiments, L is -Cy-(C₁₋₁₀ aliphatic)-NR-Cy-. In some embodiments, L is -Cy-(C₁₋₁₀ aliphatic)-Cy-NR—(C₁₋₁₀ aliphatic)-. In some embodiments, L is -Cy-(C₁₋₁₀ aliphatic)-NR-Cy-(C₁₋₁₀ aliphatic)-.

In some embodiments, L is —CONR—(C₁₋₁₀ aliphatic)-. In some embodiments, L is —(C₁₋₁₀ aliphatic)-CONR—(C₁₋₁₀ aliphatic)-. In some embodiments, L is —(C₁₋₁₀ aliphatic)-CONR—(CH2CH2O)₁₋₁₀ CH₂CH₂—. In some embodiments, L is -Cy-CONR—(C₁₋₁₀ aliphatic). In some embodiments, L is -Cy-(C₁₋₁₀ aliphatic)-CONR—. In some embodiments, L is -Cy-(C₁₋₁₀ aliphatic)-CONR—(C₁₋₁₀ aliphatic)-. In some embodiments, L is —(C₁₋₁₀ aliphatic)-Cy-CONR—(C₁₋₁₀ aliphatic)-. In some embodiments, L is —(C₁₋₁₀ aliphatic)-Cy-(C₁₋₁₀ aliphatic)-CONR—. In some embodiments, L is —(C₁₋₁₀ aliphatic)-Cy-(C₁₋₁₀ aliphatic)-CONR—(C₁₋₁₀ aliphatic)-. In some embodiments, L is -Cy-(C₁₋₁₀ aliphatic)-Cy-CONR—. In some embodiments, L is -Cy-(C₁₋₁₀ aliphatic)-CONR-Cy-. In some embodiments, L is -Cy-(C₁₋₁₀ aliphatic)-Cy- CONR—(C₁₋₁₀ aliphatic)-. In some embodiments, L is -Cy-(C₁₋₁₀ aliphatic)-CONR-Cy-(C₁₋₁₀ aliphatic)-.

In certain embodiments, L is any bivalent linker provided in WO 2024/064080, incorporated herein by reference in its entirey.

### 4. Compounds

In a first embodiment, provided is a compound of structural Formula I:

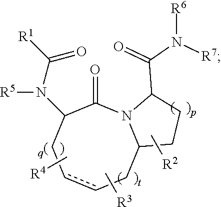

or a pharmaceutically acceptable salt thereof, wherein the variables are as described above.

In a second embodiment, R⁶ and R⁷ in the compound of Formula I or pharmaceutically acceptable salt thereof together with the nitrogen atom to which they are attached form a 4- to 14-membered monocyclic or bicyclic heterocyclyl or a 5- to 12-membered monocyclic or bicyclic heteroaryl, each of which being optionally substituted with R¹² and/or, as valency permits, optionally further substituted with 1 to 3 groups selected from R^{Q}.

In a third embodiment, the compound of Formula I is of the structural Formula II:

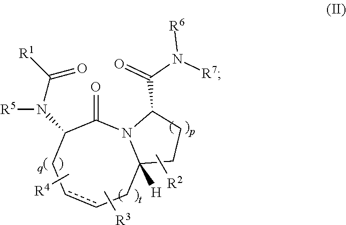

or a pharmaceutically acceptable salt thereof, wherein the variables are as described above for Formula I or the second embodiment.

In a fourth embodiment, the compound of Formula I, or pharmaceutically acceptable salt thereof, is of the structural Formula III, IV, V, or VII:

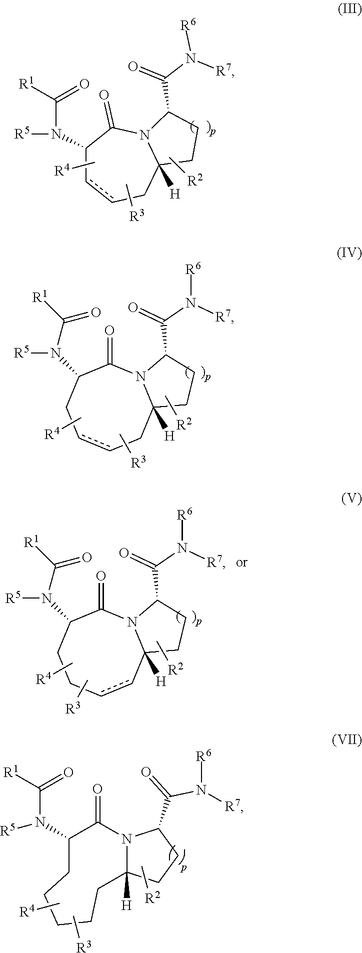

wherein the variables are as described above for Formula I or the second embodiment.

In a fifth embodiment, the compound of Formula I, or pharmaceutically acceptable salt thereof, is of the structural Formula VIII, VIII′, IX, X, XI, XII, or XIII:

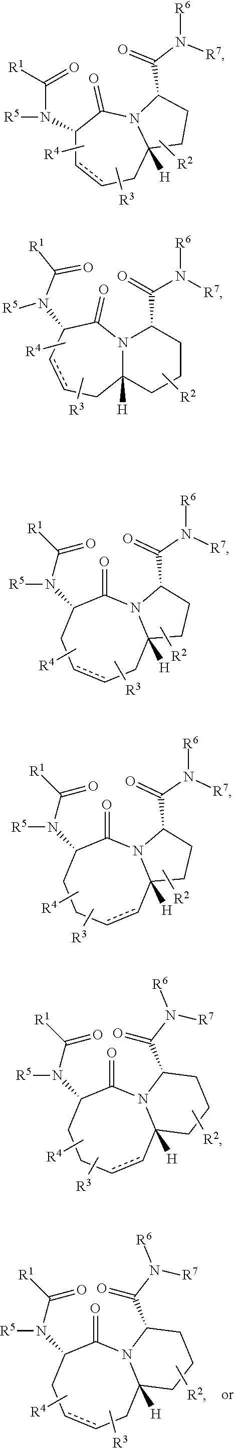

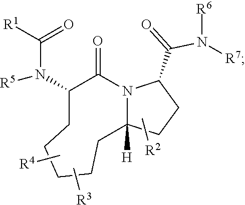

wherein the variables are as described above for Formula I or the second embodiment.

In a sixth embodiment, the compound of Formula I, or pharmaceutically acceptable salt thereof, is of the structural Formula XIV, XV, XVI, XVI′, XVII, XVIII, XX, XXI, XXII, XXIII, XXIV, XXV, XXVI, XXVII, or XXVIII:

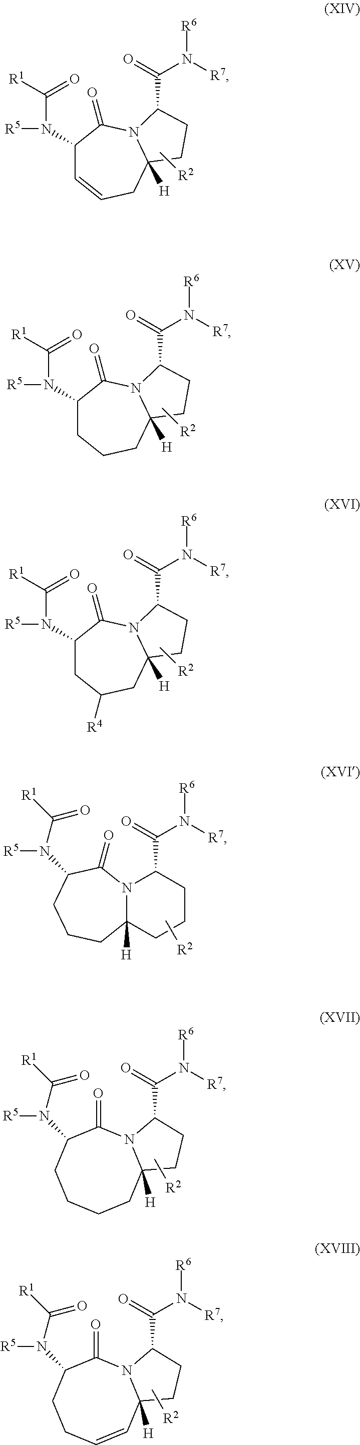

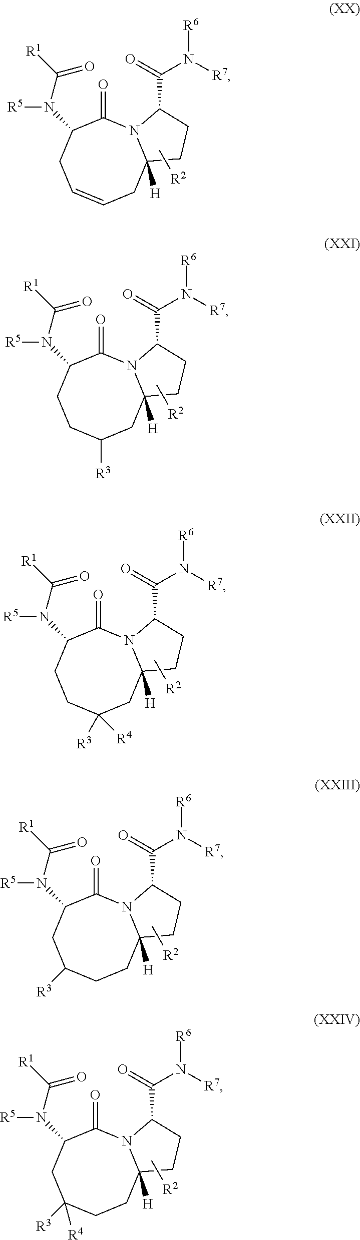

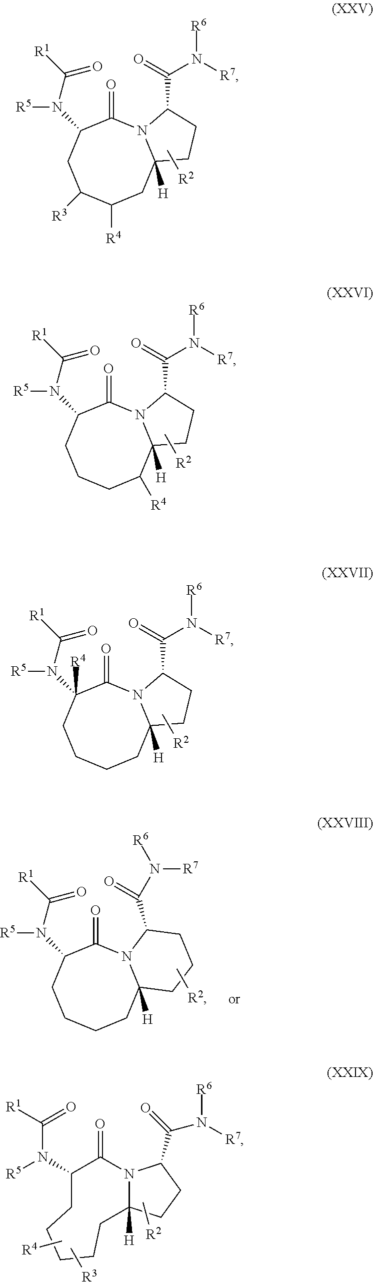

wherein the variables are as described above for Formula I or the second embodiment.

In a seventh embodiment, the compound of Formula I, or pharmaceutically acceptable salt thereof, is of the structural Formula XXX, XXXI, XXXII, XXXIII XXXIV, XXXV, XXXVI, or XXVII:

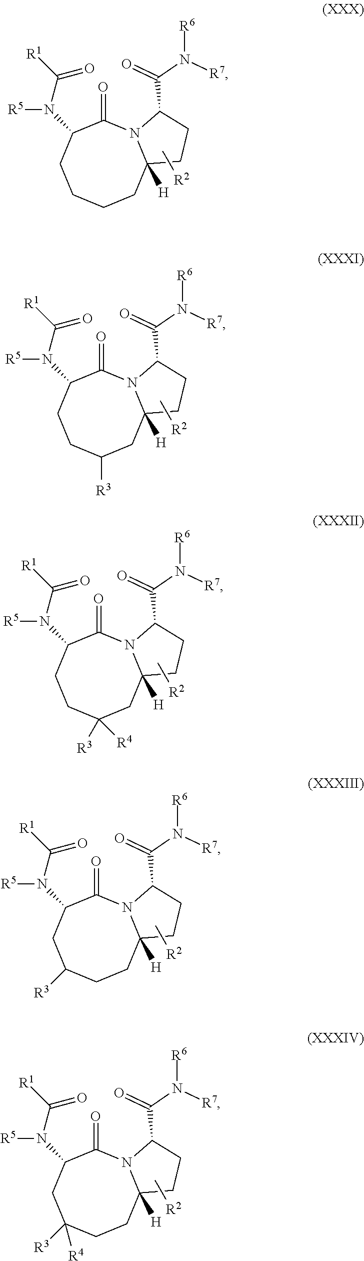

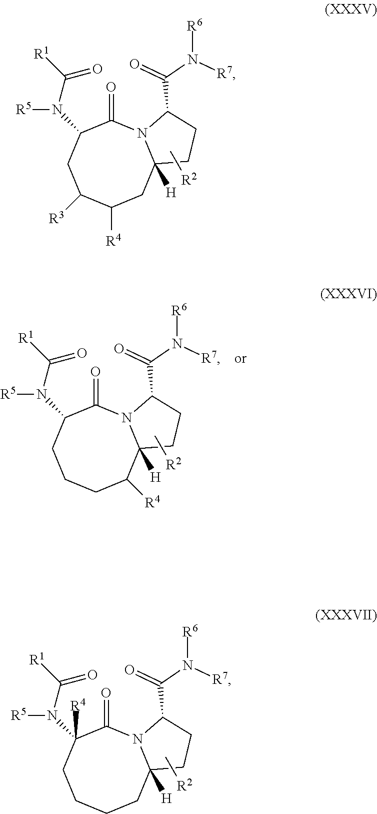

wherein the variables are as described above for Formula I or the second embodiment.

In an eighth embodiment, R³ in the compound ofany one of Formulae I, II, III, IV, V, VII, VIII, IX, X, XI, XII, XIII, XXI, XXII, XXIII, XXIV, XXV, XXIX, XXXI, XXXII, XXXIII, XXXIV, and XXXV or pharmaceutically acceptable salt thereof is selected from hydrogen, (C₁-C₄)alkyl, hydroxyl, (C₁-C₄)alkoxy, —(C₁-C₄)alkylphenyl, and 4- to 6-membered heterocyclyl; or R³ and R⁴ are taken together on the same carbon atom to form a (C₃-C₆)cycloalkyl, wherein the remaining variables are as described above for Formula I or the second embodiment. Alternatively, as part of an eighth embodiment, R⁴ in the compound of any one of Formulae I, II, III, IV, V, VII, VIII, IX, X, XI, XII, XIII, XXI, XXII, XXIII, XXIV, XXV, XXIX, XXXI, XXXII, XXXIII, XXXIV, and XXXV or pharmaceutically acceptable salt thereof is selected from hydrogen (C₁-C₄)alkyl, and hydroxyl; or R³ and R⁴ are taken together on the same carbon atom to form a (C₃-C₆)cycloalkyl, wherein the remaining variables are as described above for Formula I or the second embodiment. In another alternative, as part of an eighth embodiment, R³ in the compound of any one of Formulae I, II, III, IV, V, VII, VIII, IX, X, XI, XII, XIII, XXI, XXII, XXIII, XXIV, XXV, XXIX, XXXI, XXXII, XXXIII, XXXIV, and XXXV or pharmaceutically acceptable salt thereof is selected from hydrogen, (C₁-C₂)alkyl, hydroxyl, (C₁-C₂)alkoxy, benzyl, and azetidinyl; or R³ and R⁴ are taken together on the same carbon atom to form a cyclopropyl, wherein the remaining variables are as described above for Formula I or the second embodiment. In yet another alternative, as part of an eighth embodiment, R³ and R⁴ in the compound of any one of Formulae I, II, III, IV, V, VII, VIII, IX, X, XI, XII, XIII, XXI, XXII, XXIII, XXIV, XXV, XXIX, XXXI, XXXII, XXXIII, XXXIV, and XXXV or pharmaceutically acceptable salt thereof are hydrogen, wherein the remaining variables are as described above for Formula I or the second embodiment.

In a ninth embodiment, R² in the compound of any one of Formulae I, II, III, IV, V, VII, VIII, VIII′, IX, X, XI, XII, XIII, XIV, XV, XVI, XVI′, XVII, XVIII, XX, XXI, XXII, XXIII, XXIV, XXV, XXVI, XXVII, XXVIII, XXIX, XXX, XXXI, XXXII, XXXIII XXXIV, XXXV, XXXVI, or XXXVII or pharmaceutically acceptable salt thereof is selected from hydrogen and hydroxyl, wherein the remaining variables are as described above for Formula I or any one of the second and/or eighth embodiments. Alternatively, as part of a ninth embodiment, R² in the compound of any one of Formulae I, II, III, IV, V, VII, VIII, VIII′, IX, X, XI, XII, XIII, XIV, XV, XVI, XVI′, XVII, XVIII, XX, XXI, XXII, XXIII, XXIV, XXV, XXVI, XXVII, XXVIII, XXIX, XXX, XXXI, XXXII, XXXIII XXXIV, XXXV, XXXVI, or XXXVII or pharmaceutically acceptable salt thereof is hydrogen, wherein the remaining variables are as described above for Formula I or any one of the second and/or eighth embodiments.

In a tenth embodiment, R⁵ in the compound of any one of Formulae I, II, III, IV, V, VII, VIII, IX, X, XI, XII, XIII, XIV, XV, XVI, XVI′, XVII, XVIII, VIII′, XX, XXI, XXII, XXIII, XXIV, XXV, XXVI, XXVII, XXVIII, XXIX, XXX, XXXI, XXXII, XXXIII XXXIV, XXXV, XXXVI, or XXXVII is hydrogen, wherein the remaining variables are as described above for Formula I or any one of the second, eighth and/or ninth embodiments.

In an eleventh embodiment, R¹ in the compound of any one of Formulae I, II, III, IV, V, VII, VIII, VIII′, IX, X, XI, XII, XIII, XIV, XV, XVI, XVI′, XVII, XVIII, XX, XXI, XXII, XXIII, XXIV, XXV, XXVI, XXVII, XXVIII, XXIX, XXX, XXXI, XXXII, XXXIII XXXIV, XXXV, XXXVI, or XXXVII or pharmaceutically acceptable salt thereof is selected from 8- to 10-membered fused bicyclic heteroaryl and aryl, each of which are substituted with —CR^{1a}R^{2a}P(O)OR^{1b}OR^{2b} or —CR^{1a}R^{2a}P(O)[OR^{1b}][NH(AA)C(O)OR^{T}], wherein the remaining variables are as described above for Formula I or any one of the second and/or eighth to tenth embodiments. Alternatively, as part of an eleventh embodiment, R¹ in the compound of any one of Formulae I, II, III, IV, V, VII, VIII, VIII′, IX, X, XI, XII, XIII, XIV, XV, XVI, XVI′, XVII, XVIII, XX, XXI, XXII, XXIII, XXIV, XXV, XXVI, XXVII, XXVIII, XXIX, XXX, XXXI, XXXII, XXXIII XXXIV, XXXV, XXXVI, or XXXVII or pharmaceutically acceptable salt thereof is selected from benzothiophenyl, indolyl, and naphthalenyl, each of which are substituted with —CR^{1a}R^{2a}P(O)OR^{1b}OR^{2b} or —CR^{1a}R^{2a}P(O)[OR^{1b}][NH(AA)C(O)OR^{T}], wherein the remaining variables are as described above for Formula I or any one of the second and/or eighth to tenth embodiments. In another alternative, as part of an eleventh embodiment, R¹ in the compound of any one of Formulae I, II, III, IV, V, VII, VIII, VIII′, IX, X, XI, XII, XIII, XIV, XV, XVI, XVI′, XVII, XVIII, XX, XXI, XXII, XXIII, XXIV, XXV, XXVI, XXVII, XXVIII, XXIX, XXX, XXXI, XXXII, XXXIII XXXIV, XXXV, XXXVI, or XXXVII or pharmaceutically acceptable salt thereof is selected from,

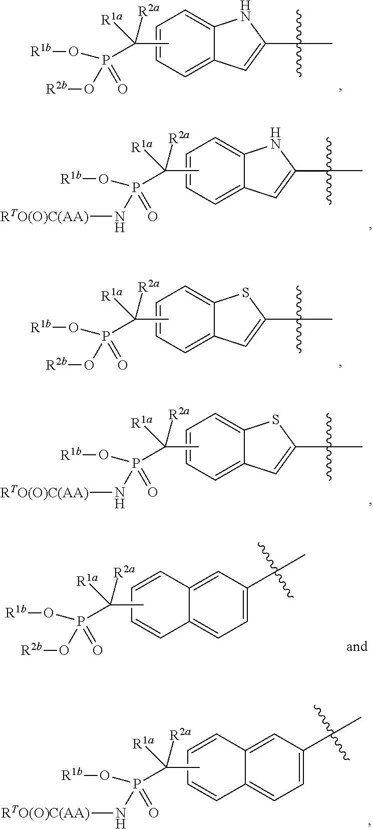

wherein the remaining variables are as described above for Formula I or any one of the second and/or eighth to tenth embodiments. In yet another alternative, as part of an eleventh embodiment, R¹ in the compound of any one of Formulae I, II, III, IV, V, VII, VIII, VIII′, IX, X, XI, XII, XIII, XIV, XV, XVI, XVI′, XVII, XVIII, XX, XXI, XXII, XXIII, XXIV, XXV, XXVI, XXVII, XXVIII, XXIX, XXX, XXXI, XXXII, XXXIII XXXIV, XXXV, XXXVI, or XXXVII or pharmaceutically acceptable salt thereof is

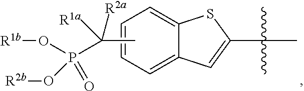

wherein the remaining variables are as described above for Formula I or any one of the second and/or eighth to tenth embodiments.

In a twelfth embodiment, R^{1a} in the compound of any one of Formulae I, II, III, IV, V, VII, VIII, VIII′, IX, X, XI, XII, XIII, XIV, XV, XVI, XVI′, XVII, XVIII, XX, XXI, XXII, XXIII, XXIV, XXV, XXVI, XXVII, XXVIII, XXIX, XXX, XXXI, XXXII, XXXIII XXXIV, XXXV, XXXVI, or XXXVII or pharmaceutically acceptable salt thereof is hydrogen and R^{2a} is fluoro or R^{1a} is fluoro and R^{2a} is fluoro, wherein the remaining variables are as described above for Formula I or any one of the second and/or eighth to eleventh embodiments. Alternatively, as part of a fourteenth embodiment, R^{1a} in the compound of any one of Formulae I, II, III, IV, V, VII, VIII, VIII′, IX, X, XI, XII, XIII, XIV, XV, XVI, XVI′, XVII, XVIII, XX, XXI, XXII, XXIII, XXIV, XXV, XXVI, XXVII, XXVIII, XXIX, XXX, XXXI, XXXII, XXXIII XXXIV, XXXV, XXXVI, or XXXVII or pharmaceutically acceptable salt thereof is fluoro and R^{2a} is fluoro, wherein the remaining variables are as described above for Formula I or any one of the second and/or eighth to eleventh embodiments.

In a thirteenth embodiment, R^{1b} and R^{2b} in the compound of any one of Formulae I, II, III, IV, V, VII, VIII, VIII′, IX, X, XI, XII, XIII, XIV, XV, XVI, XVI′, XVII, XVIII, XX, XXI, XXII, XXIII, XXIV, XXV, XXVI, XXVII, XXVIII, XXIX, XXX, XXXI, XXXII, XXXIII XXXIV, XXXV, XXXVI, or XXXVII or pharmaceutically acceptable salt thereof are each independently selected from hydrogen, (C₁-C₄)alkyl, —[(C₁-C₄)alkyl]-OC(O)—[(C₁-C₄)alkyl], —[(C₁-C₄)alkyl]-OC(O)O—[(C₁-C₄)alkyl], —[(C₁-C₄)alkyl]-SC(O)—[(C₁-C₄)alkyl], —[(C₁-C₄)alkyl]-SC(O)—[halo(C₁-C₄)alkyl], —[(C₁-C₄)alkyl]-SC(O)—[(C₁-C₄)alkyl]-OH, phenyl, pyridinyl, and naphthalenyl, wherein said phenyl, pyridinyl, and naphthalenyl are each optionally and independently substituted with cyano, wherein the remaining variables are as described above for Formula I or any one of the second and/or eighth to eleventh embodiments. Alternatively, as part of a thirteenth embodiment, R^{1b} and R^{2b} in the compound of any one of Formulae I, II, III, IV, V, VII, VIII, VIII′, IX, X, XI, XII, XIII, XIV, XV, XVI, XVI′, XVII, XVIII, XX, XXI, XXII, XXIII, XXIV, XXV, XXVI, XXVII, XXVIII, XXIX, XXX, XXXI, XXXII, XXXIII XXXIV, XXXV, XXXVI, or XXXVII or pharmaceutically acceptable salt thereof are each —[(C₁-C₄)alkyl]-OC(O)—[(C₁-C₄)alkyl], wherein the remaining variables are as described above for Formula I or any one of the second and/or eighth to eleventh embodiments. In another alternative, as part of a thirteenth embodiment, R^{1b} and R^{2b} in the compound of any one of Formulae I, II, III, IV, V, VII, VIII, VIII′, IX, X, XI, XII, XIII, XIV, XV, XVI, XVI′, XVII, XVIII, XX, XXI, XXII, XXIII, XXIV, XXV, XXVI, XXVII, XXVIII, XXIX, XXX, XXXI, XXXII, XXXIII XXXIV, XXXV, XXXVI, or XXXVII or pharmaceutically acceptable salt thereof are each hydrogen, wherein the remaining variables are as described above for Formula I or any one of the second and/or eighth to eleventh embodiments.

In a fourteenth embodiment, —CR^{1a}R^{2a}P(O)OR^{1b}OR^{2b} in the compound of any one of Formulae I, II, III, IV, V, VII, VIII, VIII′, IX, X, XI, XII, XIII, XIV, XV, XVI, XVI′, XVII, XVIII, XX, XXI, XXII, XXIII, XXIV, XXV, XXVI, XXVII, XXVIII, XXIX, XXX, XXXI, XXXII, XXXIII XXXIV, XXXV, XXXVI, or XXXVII or pharmaceutically acceptable salt thereof is selected from

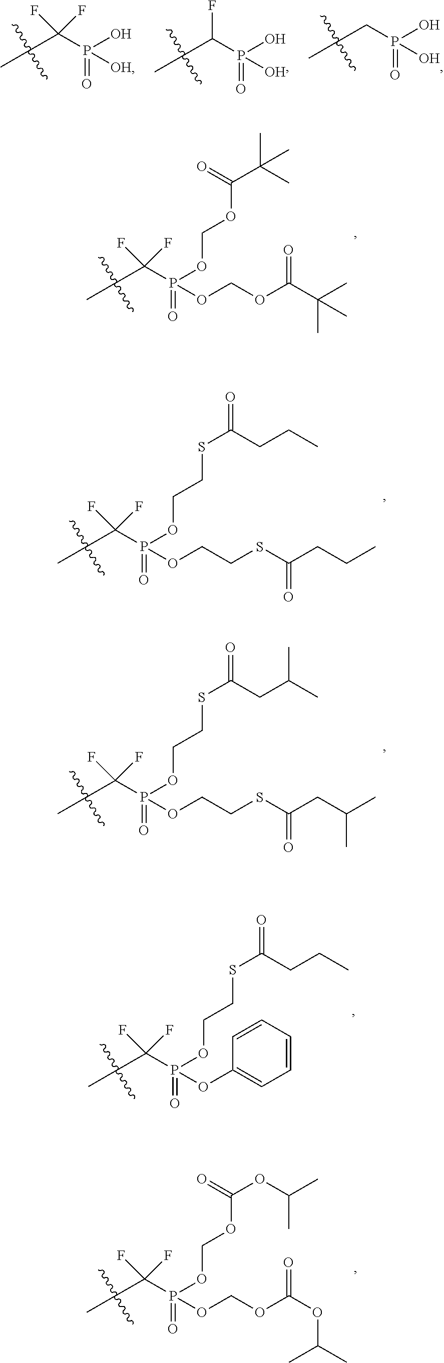

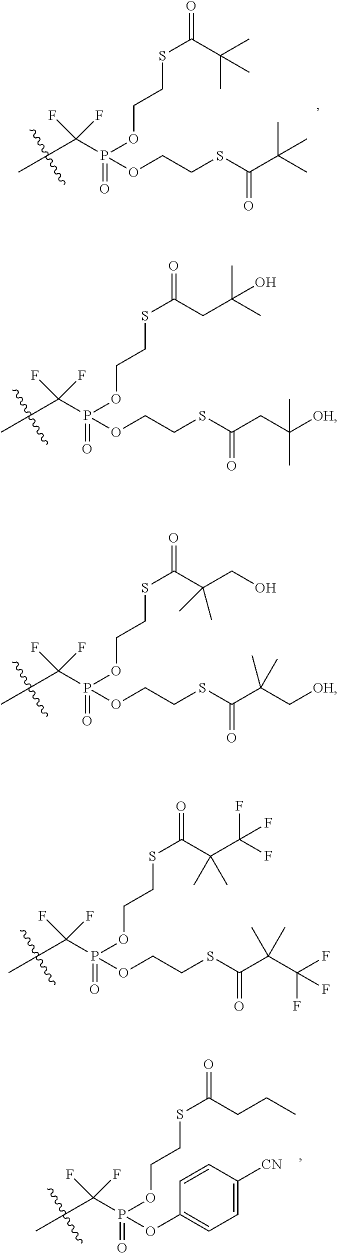 [SMILES: *C(F)(F)P(=O)(OCCSC(=O)C(C)(C)C)OCCSC(=O)C(C)(C)C ; *C(F)(F)P(=O)(OCCSC(=O)CC(C)(C)O)OCCSC(=O)CC(C)(C)O ; *C(F)(F)P(=O)(OCCSC(=O)C(C)(C)CO)OCCSC(=O)C(C)(C)CO ; *C(F)(F)P(=O)(OCCSC(=O)C(C)(C)C(F)(F)F)OCCSC(=O)C(C)(C)C(F)(F)F ; *C(F)(F)P(=O)(OCCSC(=O)CCC)Oc1ccc(C#N)cc1]

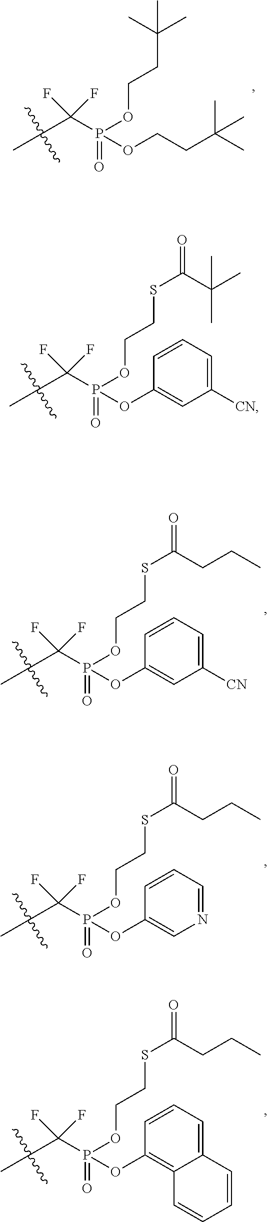 [SMILES: *C(F)(F)P(=O)(OCCC(C)(C)C)OCCC(C)(C)C ; *C(F)(F)P(=O)(OCCSC(=O)C(C)(C)C)Oc1cccc(C#N)c1 ; *C(F)(F)P(=O)(OCCSC(=O)CCC)Oc1cccc(C#N)c1 ; *C(F)(F)P(=O)(OCCSC(=O)CCC)Oc1cccnc1 ; *C(F)(F)P(=O)(OCCSC(=O)CCC)Oc1cccc2ccccc12]

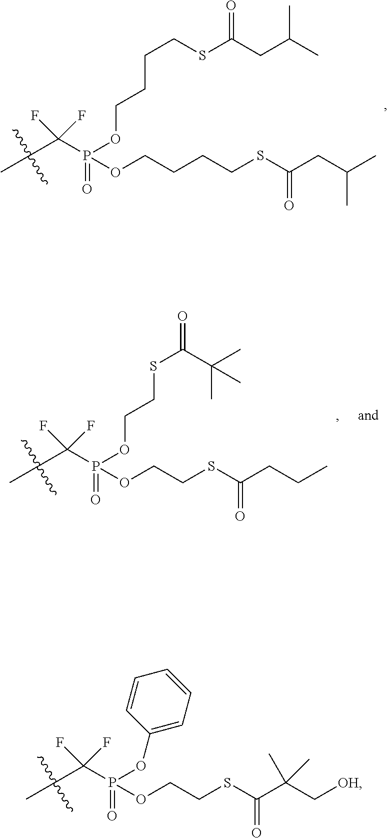 [SMILES: *C(F)(F)P(=O)(OCCCCSC(=O)CC(C)C)OCCCCSC(=O)CC(C)C ; *C(F)(F)P(=O)(OCCSC(=O)CCC)OCCSC(=O)C(C)(C)C ; *C(F)(F)P(=O)(OCCSC(=O)C(C)(C)CO)Oc1ccccc1]

wherein the remaining variables are as described above for Formula I or any one of the second and/or eighth to twelfth embodiments. Alternatively, as part of a fourteenth embodiment, —CR^{1a}R^{2a}P(O)OR^{1b}OR^{2b} in the compound of any one of Formulae I, II, III, IV, V, VII, VIII, VIII′, IX, X, XI, XII, XIII, XIV, XV, XVI, XVI′, XVII, XVIII, XX, XXI, XXII, XXIII, XXIV, XXV, XXVI, XXVII, XXVIII, XXIX, XXX, XXXI, XXXII, XXXIII XXXIV, XXXV, XXXVI, or XXXVII or pharmaceutically acceptable salt thereof is selected from:

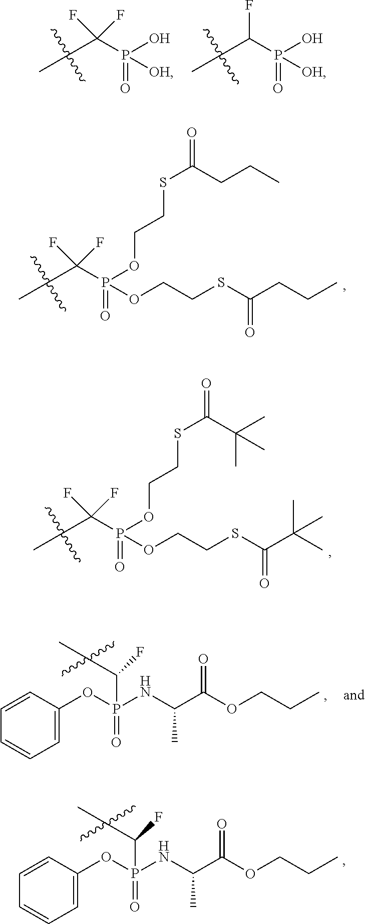 [SMILES: *C(F)(F)P(=O)(O)O ; *C(F)P(=O)(O)O ; *C(F)(F)P(=O)(OCCSC(=O)CCC)OCCSC(=O)CCC ; *C(F)(F)P(=O)(OCCSC(=O)C(C)(C)C)OCCSC(=O)C(C)(C)C ; *[C@H](F)P(=O)(N[C@@H](C)C(=O)OCCC)Oc1ccccc1 ; *[C@@H](F)P(=O)(N[C@@H](C)C(=O)OCCC)Oc1ccccc1]

wherein the remaining variables are as described above for Formula I or any one of the second and/or eighth to twelfth embodiments.

In a fourteenth embodiment, R⁶ and R⁷ in the compound of any one of Formulae I, II, III, IV, V, VII, VIII, VIII′, IX, X, XI, XII, XIII, XIV, XV, XVI, XVI′, XVII, XVIII, XX, XXI, XXII, XXIII, XXIV, XXV, XXVI, XXVII, XXVIII, XXIX, XXX, XXXI, XXXII, XXXIII XXXIV, XXXV, XXXVI, or XXXVII or pharmaceutically acceptable salt thereof together with the nitrogen atom to which they are attached form a 4- to 9-membered monocyclic or bicyclic heterocyclyl substituted with R¹² and/or, as valency permits, optionally further substituted with 1 to 3 groups selected from R^{Q}, wherein the remaining variables are as described above for Formula I or any one of the second and/or eighth to thirteenth embodiments. Alternatively, as part of a fourteenth embodiment, R⁶ and R⁷ in the compound of any one of Formulae I, II, III, IV, V, VII, VIII, VIII′, IX, X, XI, XII, XIII, XIV, XV, XVI, XVI′, XVII, XVIII, XX, XXI, XXII, XXIII, XXIV, XXV, XXVI, XXVII, XXVIII, XXIX, XXX, XXXI, XXXII, XXXIII XXXIV, XXXV, XXXVI, or XXXVII or pharmaceutically acceptable salt thereof together with the nitrogen atom to which they are attached form pyrrolidinyl, azetidinyl, or 4-azaspiro[2.4]heptanyl, each substituted with R¹² and/or, as valency permits, optionally further substituted with 1 to 3 groups selected from R^{Q}, wherein the remaining variables are as described above for Formula I or any one of the second and/or eighth to thirteenth embodiments.

In a fifteenth embodiment, R^{Q} in the compound of any one of Formulae I, II, III, IV, V, VII, VIII, VIII′, IX, X, XI, XII, XIII, XIV, XV, XVI, XVI′, XVII, XVIII, XX, XXI, XXII, XXIII, XXIV, XXV, XXVI, XXVII, XXVIII, XXIX, XXX, XXXI, XXXII, XXXIII XXXIV, XXXV, XXXVI, or XXXVII or pharmaceutically acceptable salt thereof is cyano or 5- to 7-membered heteroaryl, wherein the remaining variables are as described above for Formula I or any one of the second and/or eighth to fourteenth embodiments. Alternatively, as part of a fifteenth embodiment, R^{Q} in the compound of any one of Formulae I, II, III, IV, V, VII, VIII, VIII′, IX, X, XI, XII, XIII, XIV, XV, XVI, XVI′, XVII, XVIII, XX, XXI, XXII, XXIII, XXIV, XXV, XXVI, XXVII, XXVIII, XXIX, XXX, XXXI, XXXII, XXXIII XXXIV, XXXV, XXXVI, or XXXVII or pharmaceutically acceptable salt thereof is cyano or pyridinyl, wherein the remaining variables are as described above for Formula I or any one of the second and/or eighth to fourteenth embodiments.

In a sixteenth embodiment, R¹² in the compound of any one of Formulae I, II, III, IV, V, VII, VIII, VIII′, IX, X, XI, XII, XIII, XIV, XV, XVI, XVI′, XVII, XVIII, XX, XXI, XXII, XXIII, XXIV, XXV, XXVI, XXVII, XXVIII, XXIX, XXX, XXXI, XXXII, XXXIII XXXIV, XXXV, XXXVI, or XXXVII or pharmaceutically acceptable salt thereof is selected from —NHC(O)R^{10C}E, —OR^{10C}E, phenyl, pyridinyl, and pyridin-2(1H)-one, each substituted with R^{10C}E, —OR^{10C}E, —NH R^{10C}E, or —[N(C₁-C₄)alkyl]R^{10C}E, wherein the remaining variables are as described above for Formula I or any one of the second and/or eighth to fifteenth embodiments.

In a seventeenth embodiment, R^{10C} in the compound of any one of Formulae I, II, III, IV, V, VII, VIII, VIII′, IX, X, XI, XII, XIII, XIV, XV, XVI, XVI′, XVII, XVIII, XX, XXI, XXII, XXIII, XXIV, XXV, XXVI, XXVII, XXVIII, XXIX, XXX, XXXI, XXXII, XXXIII XXXIV, XXXV, XXXVI, or XXXVII or pharmaceutically acceptable salt thereof is selected from (C₁-C₁₀)alkylene and (C₂-C₁₀)alkynelene each optionally substituted with one or more groups selected from halo and oxo, and wherein said (C₁-C₆)alkylene and (C₂-C₁₀)alkynelene may also be optionally interrupted by one or more heteroatoms selected from S, N, and O, and/or optionally interrupted by one or more rings selected from 5- to 7-membered heteroaryl, (C₃-C₆)cycloakyl, phenyl, and 4- to 7-membered heterocyclyl, wherein the remaining variables are as described above for Formula I or any one of the second and/or eighth to sixteenth embodiments. Alternatively, as part of a seventeenth embodiment, R^{10C} in the compound of any one of Formulae I, II, III, IV, V, VII, VIII, VIII′, IX, X, XI, XII, XIII, XIV, XV, XVI, XVI′, XVII, XVIII, XX, XXI, XXII, XXIII, XXIV, XXV, XXVI, XXVII, XXVIII, XXIX, XXX, XXXI, XXXII, XXXIII XXXIV, XXXV, XXXVI, or XXXVII or pharmaceutically acceptable salt thereof is selected from (C₁-C₁₀)alkylene, (C₂-C₁₀)alkynelene, —(CH₂)ₘC(O)NH[(CH₂)O]ᵥ(CH₂)ₙ, —(CH₂)O[(CH₂)O]ᵥ(CH₂)ₙ, —(C₂-C₈)alkynelene[4- to 7-membered heterocyclyl], 4- to 7-membered heterocyclyl, [(CH₂)O]ᵥ(CH₂)ₙ, and —(CH₂)ₙC(O)NH[4- to 7-membered heterocyclyl], wherein m, n, and v are each independently selected from 0, 1, 2, 3, 4, 5, and 6, wherein the remaining variables are as described above for Formula I or any one of the second and/or eighth to sixteenth embodiments. In another alternative, as part of a seventeenth embodiment, R^{10C} in the compound of any one of Formulae I, II, III, IV, V, VII, VIII, VIII′, IX, X, XI, XII, XIII, XIV, XV, XVI, XVI′, XVII, XVIII, XX, XXI, XXII, XXIII, XXIV, XXV, XXVI, XXVII, XXVIII, XXIX, XXX, XXXI, XXXII, XXXIII XXXIV, XXXV, XXXVI, or XXXVII or pharmaceutically acceptable salt thereof is selected from

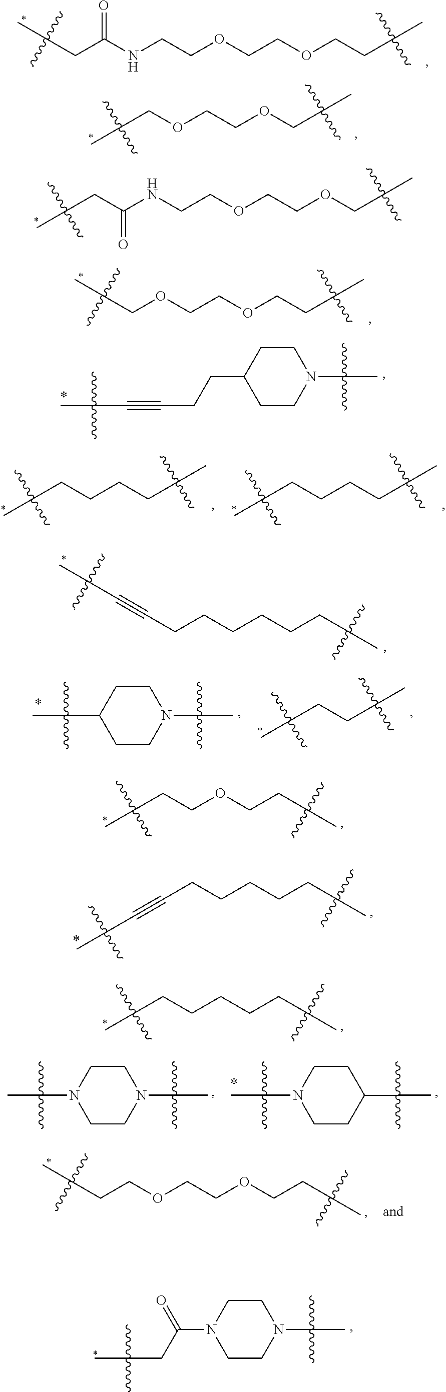

wherein the asterisk indicates the position to which E is attached, and wherein the remaining variables are as described above for Formula I or any one of the second and/or eighth to sixteenth embodiments.

In an eighteenth embodiment, E in the compound of any one of Formulae I, II, III, IV, V, VII, VIII, VIII′, IX, X, XI, XII, XIII, XIV, XV, XVI, XVI′, XVII, XVIII, XX, XXI, XXII, XXIII, XXIV, XXV, XXVI, XXVII, XXVIII, XXIX, XXX, XXXI, XXXII, XXXIII XXXIV, XXXV, XXXVI, or XXXVII or pharmaceutically acceptable salt thereof is selected from a CRBN, VHL, IAP, or MDM2 based E3 binder, wherein the remaining variables are as described above for Formula I or any one of the second and/or eighth to seventeenth embodiments. Alternatively, as part of an eighteenth embodiment, E in the compound of any one of Formulae I, II, III, IV, V, VII, VIII, VIII′, IX, X, XI, XII, XIII, XIV, XV, XVI, XVI′, XVII, XVIII, XX, XXI, XXII, XXIII, XXIV, XXV, XXVI, XXVII, XXVIII, XXIX, XXX, XXXI, XXXII, XXXIII XXXIV, XXXV, XXXVI, or XXXVII or pharmaceutically acceptable salt thereof is selected from

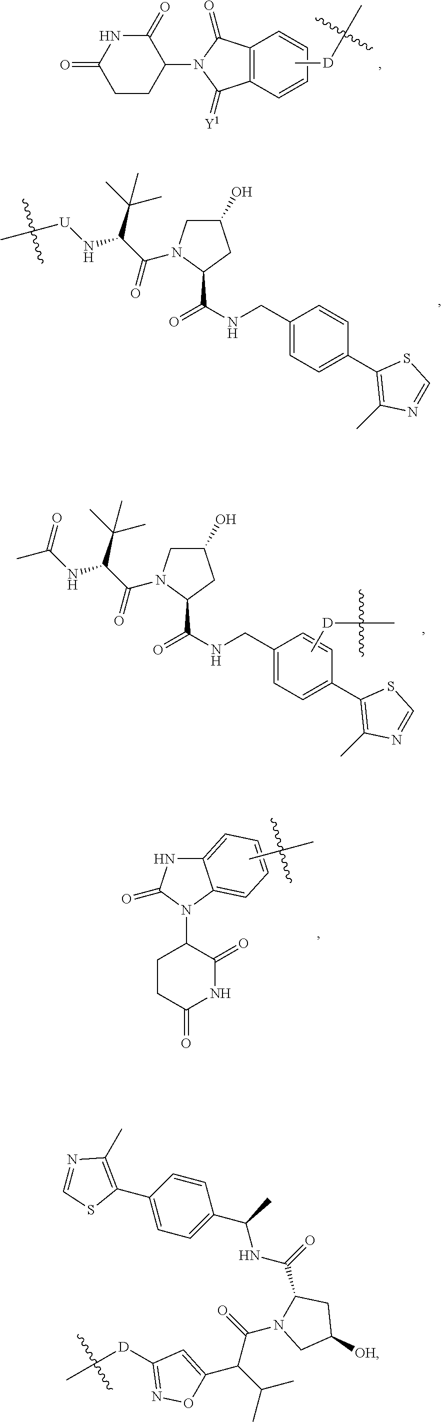

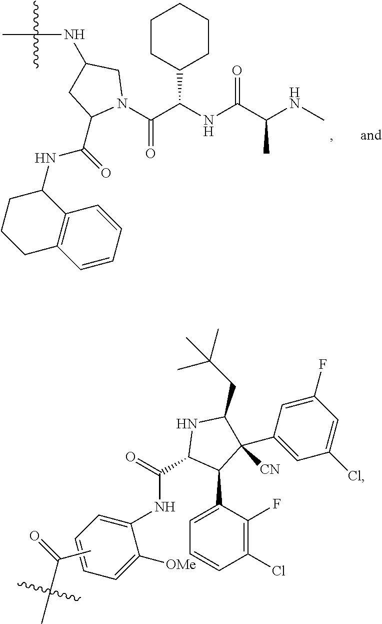

wherein:
Y and Y¹ are each independently is O or CH₂;
D is O, NH, or a bond;
U is absent or C(O); and
R is halo or cyano, wherein the remaining variables are as described above for Formula I or any one of the second and/or eighth to seventeenth embodiments. In another alternative, as part of an eighteenth embodiment, Ein the compound of any one of Formulae I, II, III, IV, V, VII, VIII, VIII′, IX, X, XI, XII, XIII, XIV, XV, XVI, XVI′, XVII, XVIII, XX, XXI, XXII, XXIII, XXIV, XXV, XXVI, XXVII, XXVIII, XXIX, XXX, XXXI, XXXII, XXXIII XXXIV, XXXV, XXXVI, or XXXVII or pharmaceutically acceptable salt thereof is selected from

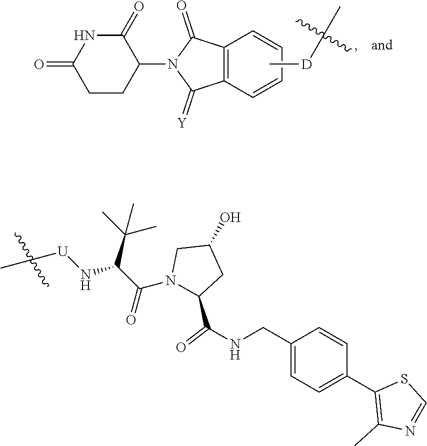

wherein the remaining variables are as described above for Formula I or any one of the second and/or eighth to seventeenth embodiments.

Compounds having the Formula I are further described in the Exemplification and are included in the present disclosure. Pharmaceutically acceptable salts thereof as well as the neutral forms are included.

### 4. Uses, Formulation and Administration

The compounds and compositions described herein are generally useful for modulating the activity of STAT proteins, in particular STAT3 and/or STAT6. In some aspects, the compounds, pharmaceutical acceptable salts, and pharmaceutical compositions described herein degrade STAT3 and/or STAT6.

In some aspects, the compounds and pharmaceutical compositions described herein are useful in a condition responsive to the modulation (or degradation) of STAT3 and/or STAT6. Thus, provided herein are methods of treating a condition responsive to the modulation (e.g., degradation) of STAT3 and/or STAT6 in a subject, comprising administering to a subject in need thereof, a therapeutically effective amount of a compound described herein, or a pharmaceutically acceptable salt thereof, or a pharmaceutical composition comprising a disclosed compound or pharmaceutically acceptable salt thereof.

Also provided is the use of a compound described herein, or a pharmaceutically acceptable salt thereof, or a pharmaceutical composition comprising a disclosed compound or pharmaceutically acceptable salt thereof, for the manufacture of a medicament for treating a condition responsive to the modulation (e.g., degradation) of STAT3 and/or STAT6. Also provided is a compound described herein, or a pharmaceutically acceptable salt thereof, or a pharmaceutical composition comprising a disclosed compound or pharmaceutically acceptable salt thereof, for use in treating a condition responsive to the modulation (e.g., degradation) of STAT3 and/or STAT6.

In one aspect, the condition responsive to the modulation (e.g., degradation) of STAT3 and/or STAT6 include, but are not limited to, cancer, a neurodegenative disorder, a viral disease, an autoimmune disease, an inflammatory disorder, a hereditary disorder, a hormone-related disease, a metabolic disorder, conditions associated with organ transplantation, immunodeficiency disorders, a destructive bone disorder, a proliferative disorder, an infectious disease, a condition associated with cell death, thrombin-induced platelet aggregation, liver disease, pathologic immune conditions involving T cell activation, a cardiovascular disorder, or a CNS disorder.

In another aspect, the condition responsive to the modulation (e.g., degradation) of STAT3 and/or STAT6 include, but are not limited to, cancer (see, e.g., Turkson & Jove, Oncogene 2000, 19:6613-6626), diabetes (see. e.g., Gurzov et al., FEBS 2016, 283:3002), cardiovascular disease (see, e.g., Grote et al., Vasc. Pharmacol. 2005, 43:2005), viral disease (see, e.g., Gao et al., J Hepatol. 2012, 57(2):430), autoimmune diseases such as lupus (see, e.g., Goropevsek et al., Clin. Rev. Alleg. & Immun. 2017, 52(2):164), and rheumatoid arthritis (see, e.g., Walker & Smith, J. Rheumat. 2005, 32(9): 1650), autoinflammatory syndromes (see, e.g., Rauch et al., Jak-Stat 2013, 2(1):e23820), atherosclerosis (see, e.g., Ortiz-Munoz et al., Arterio., Thromho., Vase. Bio. 2009, 29:525), psoriasis (see, e.g., Andres et al., Exp. Derm. 2013, 22(5):323), allergic disorders (see, e.g., Oh et al., Eur. Respir. Rev. 2019, 19(115):46), inflammatory bowel disease (see. e.g., Sugimoto, World J Gastroenterol. 2008, 14(33):5110), inflammation (see, e.g., Tamiya et al., Arierio. Thrombo., Vasc. Bio. 2011, 31:980), acute and chronic gout and gouty arthritis, neurological disorders (see, e.g., Campbell, Brain Res. Rev. 2005, 48(2): 166), metabolic syndrome, immunodeficiency disorders such as AIDS and HIV (see, e.g., O'Shea et al., N. Engl. J.Med. 2013, 368:161), destructive bone disorders (see, e.g., Jatiani et al., Genes & Can. 2011, 1(10):979), osteoarthritis, proliferative disorders, Waldenstrom's Macroglobulinemia (see, e.g., Hodge et al., Blood 2014, 123(7):1055) infectious diseases, conditions associated with cell death, pathologic immune conditions involving T cell activation, and CNS disorders.

Proliferative disorders, include, but are not limited to a benign or malignant tumor, solid tumor, liquid tumor, carcinoma of the brain, kidney, liver, adrenal gland, bladder, breast, stomach, gastric tumors, ovaries, colon, rectum, prostate, pancreas, lung, vagina, cervix, testis, genitourinary tract, esophagus, larynx, skin, bone or thyroid, sarcoma, glioblastomas, neuroblastomas, multiple myeloma, gastrointestinal cancer, especially colon carcinoma or colorectal adenoma, a tumor of the neck and head, an epidermal hyperproliferation, psoriasis, prostate hyperplasia, a neoplasia, a neoplasia of epithelial character, adenoma, adenocarcinoma, keratoacanthoma, epidermoid carcinoma, large cell carcinoma, non-small-cell lung carcinoma, lymphomas, Hodgkins and Non-Hodgkins, a mammary carcinoma, follicular carcinoma, undifferentiated carcinoma, papillary carcinoma, seminoma, melanoma, an IL-I driven disorder, an MyD88 driven disorder, Smoldering of indolent multiple myeloma, or hematological malignancies (including leukemia, diffuse large B-cell lymphoma (DLBCL), ABC DLBCL, chronic lymphocytic leukemia (CLL), chronic lymphocytic lymphoma, primary effusion lymphoma, Burkitt lymphoma/leukemia, acute lymphocytic leukemia, B-cell prolymphocytic leukemia, lymphoplasmacytic lymphoma, Waldenstrom's macroglobulinemia (WM), splenic marginal zone lymphoma, multiple myeloma, plasmacytoma, intravascular large B-cell lymphoma).

In some embodiments, the cancer to be treated is selected from glioma, breast cancer, prostate cancer, head and neck squamous cell carcinoma, skin melanomas, ovarian cancer, malignant peripheral nerve sheath tumors (MPNST), and pancreatic cancer. In other embodiments, the cancer to be treated is cancer selected from glioma, breast cancer, prostate cancer, head and neck squamous cell carcinoma, skin melanomas, ovarian cancer, malignant peripheral nerve shealth tumors (MPNST), pancreatic cancer, non-small cell lung cancer (NSCLC) including EGFR-mutant NSCLC, urothelial cancer, liver cancer, bile duct cancer, kidney cancer, colon cancer, esophageal cancer, gastric cancer, gastrointestinal stromal tumors, and hematological malignancies include lymphomas, leukemias, myelomas, myeloproliferative neoplasms and myelodysplastic syndromes. In other embodiments, the cancer is selected from solid tumors (e.g., prostate cancer, renal cancer, hepatic cancer, pancreatic cancer, gastric cancer, breast cancer, lung cancer, cancers of the head and neck, thyroid cancer, glioblastoma, Kaposi [symbol] sarcoma, Castleman [symbol] disease, uterine leiomyosarcoma, melanoma etc.), hematological cancers (e.g., lymphoma, leukemia such as acute lymphoblastic leukemia (ALL), acute myelogenous leukemia (AML) or multiple myeloma), and skin cancer such as cutaneous T-cell lymphoma (CTCL) and cutaneous B-cell lymphoma. Example CTCLs include Sezary syndrome and mycosis fungoides.

Compounds, salts, and compositions described herein are also useful in the treatment of inflammatory or obstructive airways diseases, resulting, for example, in reduction of tissue damage, airways inflammation, bronchial hyperreactivity, remodeling or disease progression. Inflammatory or obstructive airways diseases include asthma of whatever type or genesis including both intrinsic (non-allergic) asthma and extrinsic (allergic) asthma, mild asthma, moderate asthma, severe asthma, bronchitic asthma, exercise-induced asthma, occupational asthma and asthma induced following bacterial infection. Treatment of asthma is also to be understood as embracing treatment of subjects, e.g. of less than 4 or 5 years of age, exhibiting wheezing symptoms and diagnosed or diagnosable as “wheezy infants”, an established patient category of major medical concern and now often identified as incipient or early-phase asthmatics.

Compounds, salts, and compositions described herein are also useful in the treatment of heteroimmune diseases including, but are not limited to, graft versus host disease, transplantation, transfusion, anaphylaxis, allergies (e.g., allergies to plant pollens, latex, drugs, foods, insect poisons, animal hair, animal dander, dust mites, or cockroach calyx), type I hypersensitivity, allergic conjunctivitis, allergic rhinitis, and atopic dermatitis.

Compounds, salts, and compositions described herein are also useful in the treatment of other inflammatory or obstructive airways diseases and conditions to which the present invention is applicable and include acute lung injury (ALI), adult/acute respiratory distress syndrome (ARDS), chronic obstructive pulmonary, airways or lung disease (COPD, COAD or COLD), including chronic bronchitis or dyspnea associated therewith, emphysema, as well as exacerbation of airways hyperreactivity consequent to other drug therapy, in particular other inhaled drug therapy. Compounds, salts, and compositions described herein are also useful in the treatment of bronchitis including, but not limited to, acute, arachidic, catarrhal, croupus, chronic or phthinoid bronchitis. Compounds, salts, and compositions described herein are also useful in the treatment of pneumoconiosis (an inflammatory, commonly occupational, disease of the lungs, frequently accompanied by airways obstruction, whether chronic or acute, and occasioned by repeated inhalation of dusts) of whatever type or genesis, including, for example, aluminosis, anthracosis, asbestosis, chalicosis, ptilosis, siderosis, silicosis, tabacosis and byssinosis.

Compounds, salts, and compositions described herein are also useful in the treatment of inflammatory or allergic conditions of the skin, for example psoriasis, contact dermatitis, atopic dermatitis, alopecia areata, erythema multiforma, dermatitis herpetiformis, scleroderma, vitiligo, hypersensitivity angiitis, urticaria, bullous pemphigoid, lupus erythematosus, systemic lupus erythematosus, pemphigus vulgaris, pemphigus foliaceus, paraneoplastic pemphigus, epidermolysis bullosa acquisita, acne vulgaris, and other inflammatory or allergic conditions of the skin.

Compounds, salts, and compositions described herein are also useful in the treatment of other diseases or conditions, such as diseases or conditions having an inflammatory component, for example, treatment of diseases and conditions of the eye such as ocular allergy, conjunctivitis, keratoconjunctivitis sicca, and vernal conjunctivitis, diseases affecting the nose including allergic rhinitis, and inflammatory disease in which autoimmune reactions are implicated or having an autoimmune component or etiology, including autoimmune hematological disorders (e.g. hemolytic anemia, aplastic anemia, pure red cell anemia and idiopathic thrombocytopenia), systemic lupus erythematosus, rheumatoid arthritis, polychondritis, scleroderma, Wegener granulamatosis, dermatomyositis, chronic active hepatitis, myasthenia gravis, Steven-Johnson syndrome, idiopathic sprue, autoimmune inflammatory bowel disease (e.g. ulcerative colitis and Crohn© disease), irritable bowel syndrome, celiac disease, periodontitis, hyaline membrane disease, kidney disease, glomerular disease, alcoholic liver disease, multiple sclerosis, endocrine opthalmopathy, Grave© disease, sarcoidosis, alveolitis, chronic hypersensitivity pneumonitis, multiple sclerosis, primary biliary cirrhosis, uveitis (anterior and posterior), Sjogren's syndrome, keratoconjunctivitis sicca and vernal keratoconjunctivitis, interstitial lung fibrosis, psoriatic arthritis, systemic juvenile idiopathic arthritis, cryopyrin-associated periodic syndrome, nephritis, vasculitis, diverticulitis, interstitial cystitis, glomerulonephritis (with and without nephrotic syndrome, e.g. including idiopathic nephrotic syndrome or minal change nephropathy), chronic granulomatous disease, endometriosis, leptospiriosis renal disease, glaucoma, retinal disease, ageing, headache, pain, complex regional pain syndrome, cardiac hypertrophy, musclewasting, catabolic disorders, obesity, fetal growth retardation, hyperchlolesterolemia, heart disease, chronic heart failure, mesothelioma, anhidrotic ectodermal dysplasia, Behcet's disease, incontinentia pigmenti, Paget's disease, pancreatitis, hereditary periodic fever syndrome, asthma (allergic and non-allergic, mild, moderate, severe, bronchitic, and exercise-induced), acute lung injury, acute respiratory distress syndrome, eosinophilia, hypersensitivities, anaphylaxis, nasal sinusitis, ocular allergy, silica induced diseases, COPD (reduction of damage, airways inflammation, bronchial hyperreactivity, remodeling or disease progression), pulmonary disease, cystic fibrosis, acid induced lung injury, pulmonary hypertension, polyneuropathy, cataracts, muscle inflammation in conjunction with systemic sclerosis, inclusion body myositis, myasthenia gravis, thyroiditis, Addison's disease, lichen planus, Type 1 diabetes, or Type 2 diabetes, appendicitis, atopic dermatitis, asthma, allergy, blepharitis, bronchiolitis, bronchitis, bursitis, cervicitis, cholangitis, cholecystitis, chronic graft rejection, colitis, conjunctivitis, Crohn's disease, cystitis, dacryoadenitis, dermatitis, dermatomyositis, encephalitis, encephalomyelitis, endocarditis, endometritis, enteritis, enterocolitis, epicondylitis, epididymitis, fasciitis, fibrositis, gastritis, gastroenteritis, Henoch-Schonlein purpura, hepatitis, hidradenitis suppurativa, immunoglobulin A nephropathy, interstitial lung disease, laryngitis, mastitis, meningitis, myelitis myocarditis, myositis, nephritis, oophoritis, orchitis, osteitis, otitis, pancreatitis, parotitis, pericarditis, peritonitis, pharyngitis, pleuritis, phlebitis, pneumonitis, pneumonia, polymyositis, proctitis, prostatitis, pyelonephritis, rhinitis, salpingitis, sinusitis, stomatitis, synovitis, tendonitis, tonsillitis, ulcerative colitis, uveitis, vaginitis, vasculitis, or vulvitis.

In some embodiments, cardiovascular diseases which can be treated according to the present methods include, but are not limited to, restenosis, cardiomegaly, atherosclerosis, myocardial infarction, ischemic stroke, congestive heart failure, angina pectoris, reocclusion after angioplasty, restenosis after angioplasty, reocclusion after aortocoronary bypass, restenosis after aortocoronary bypass, stroke, transitory ischemia, a peripheral arterial occlusive disorder, pulmonary embolism, and deep venous thrombosis.

In some embodiments, the neurodegenerative disease which can be treated according to the present methods include, but are not limited to, Alzheimer [symbol] disease, Parkinson [symbol] disease, amyotrophic lateral sclerosis, Huntington [symbol] disease, cerebral ischemia, and neurodegenerative disease caused by traumatic injury, glutamate neurotoxicity, hypoxia, epilepsy, treatment of diabetes, metabolic syndrome, obesity, organ transplantation and graft versus host disease.

In certain aspects, a pharmaceutical composition described herein is formulated for administration to a patient in need of such composition. Pharmaceutical compositions described herein may be administered orally, parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. In some embodiments, the compositions are administered orally, intraperitoneally or intravenously. Sterile injectable forms of the pharmaceutical compositions described herein may be aqueous or oleaginous suspension. These suspensions may be formulated according to techniques known in the art using suitable dispersing or wetting agents and suspending agents.

In some aspects, the pharmaceutical compositions are administered orally.

A specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease being treated. The amount of a compound described herein in the composition will also depend upon the particular compound in the pharmaceutical composition.

### Exemplification

### Preparation of Compounds

The compounds claimed herein were prepared following the procedures outlined in the following schemes. Compound names were generated using the software built into ChemDraw. To the extent that there are discrepancies between the name of a compound and its depicted structure, the depicted chemical structure is to be taken as the appropriate compound.

The following intermediates can be prepared according to the protocols described in PCT/US2023/010447, which is incorporated herein by reference in its entirety.
(3S,6S,9aS)-6-((tert-butoxycarbonyl)amino)-5-oxooctahydro-1H-pyrrolo[1,2-a]azepine-3-carboxylic acid;
(3S,6S,10aS)-6-((tert-butoxycarbonyl)amino)-5-oxodecahydropyrrolo[1,2-a]azocine-3-carboxylic acid;
4-nitrophenyl 5-((bis(2-(butyrylthio)ethoxy)phosphoryl) difluoromethyl) benzo[b]thiophene-2-carboxylate;
(difluoro(2-((perfluorophenoxy)carbonyl)benzo[b]thiophen-5-yl)methyl)phosphonic acid;
4-nitrophenyl 5-((bis(2-(pivaloylthio)ethoxy) phosphoryl)difluoromethyl) benzo[b]thiophene-2-carboxylate;
(fluoro(2-((perfluorophenoxy)carbonyl)benzo[b]thiophen-5-yl)methyl)phosphonic acid;
perfluorophenyl 5-((1R)-fluoro((((S)-1-oxo-1-propoxypropan-2-yl)amino)(phenoxy) phosphoryl)methyl)benzo[b]thiophene-2-carboxylate;
perfluorophenyl 5-((1S)-fluoro((((S)-1-oxo-1-propoxypropan-2-yl)amino)(phenoxy) phosphoryl)methyl)benzo[b]thiophene-2-carboxylate;
tert-butyl 3-(4-fluoropyridin-3-yl)azetidine-1-carboxylate; and
tert-butyl (R)-6-(2-oxo-1,2-dihydropyridin-4-yl)-4-azaspiro[2.4]heptane-4-carboxylate.

### Preparation of S,S((((difluoro(2-(((3S,6S,9aS)-3-(3-(4-(4-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)butoxy)pyridin-3-yl)azetidine-1-carbonyl)-5-oxooctahydro-1H-pyrrolo[1,2-a]azepin-6-yl)carbamoyl)benzo[b]thiophen-5-yl)methyl)phosphoryl)bis(oxy))bis(ethane-2,1-diyl)) dibutanethioate (Compound 1) and (difluoro(2-(((3S,6S,9aS)-3-(3-(4-(4-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)butoxy)pyridin-3-yl)azetidine-1-carbonyl)-5-oxooctahydro-1H-pyrrolo[1,2-a]azepin-6-yl)carbamoyl)benzo[b]thiophen-5-yl)methyl)phosphonic acid (Compound 2)

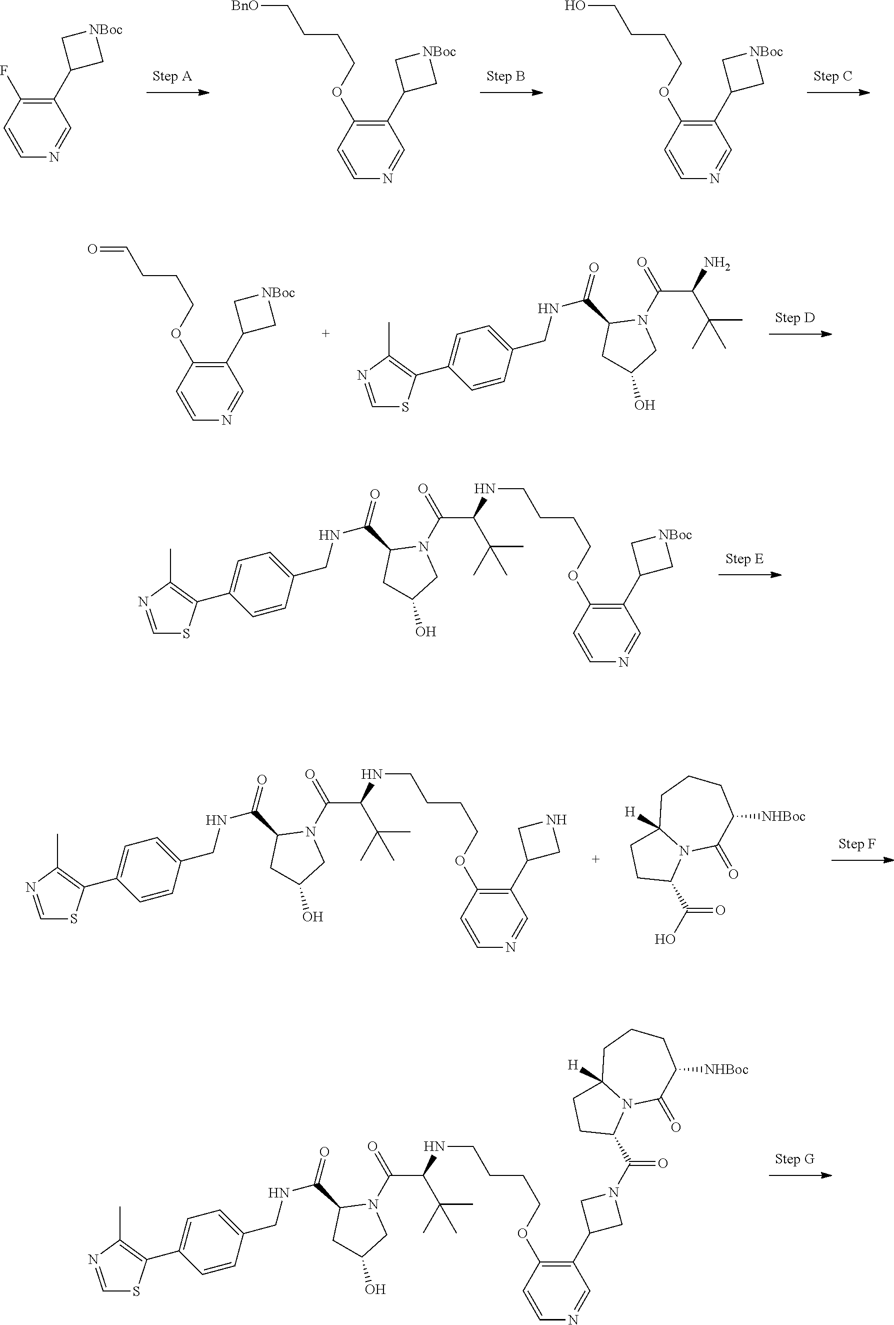

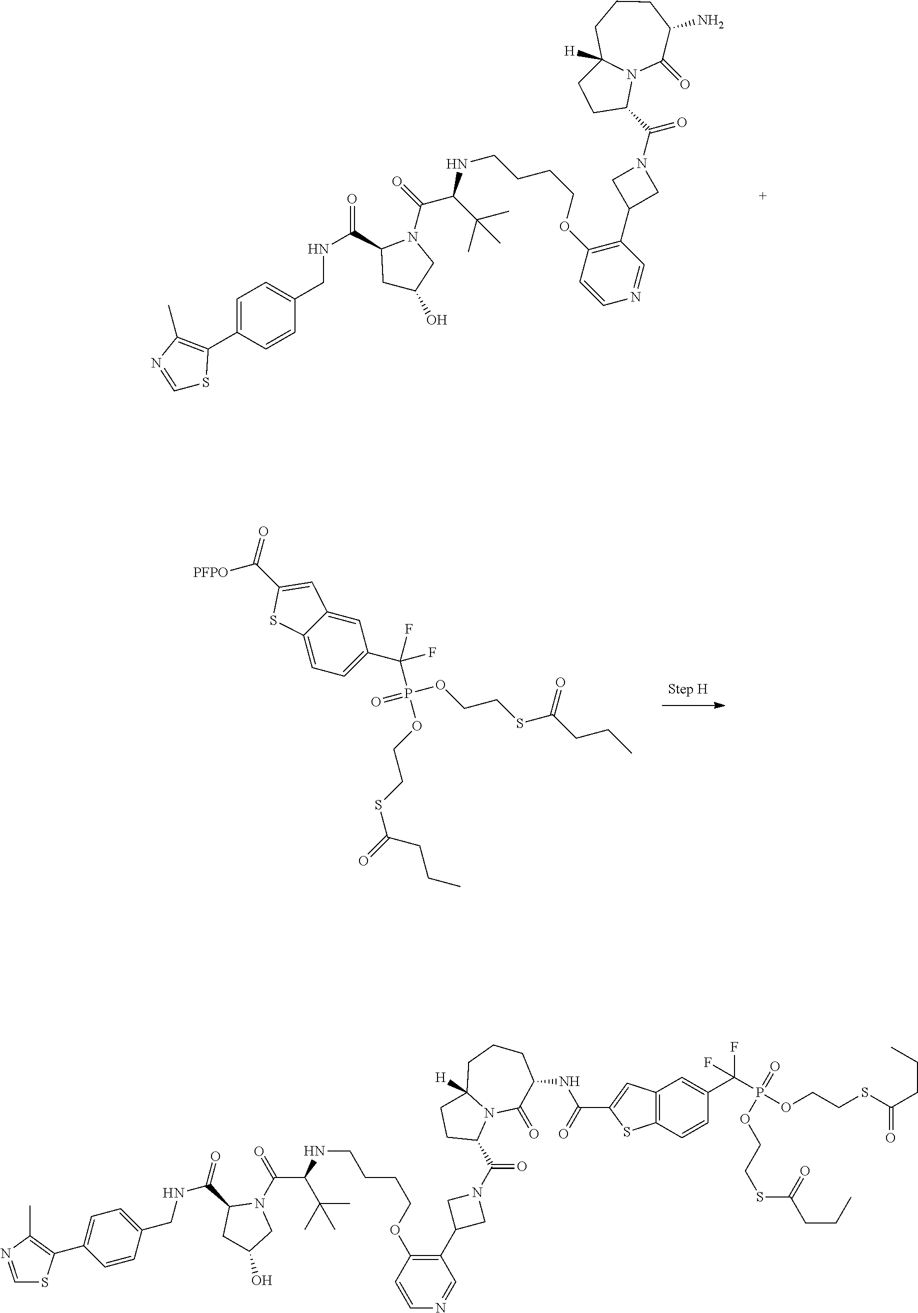

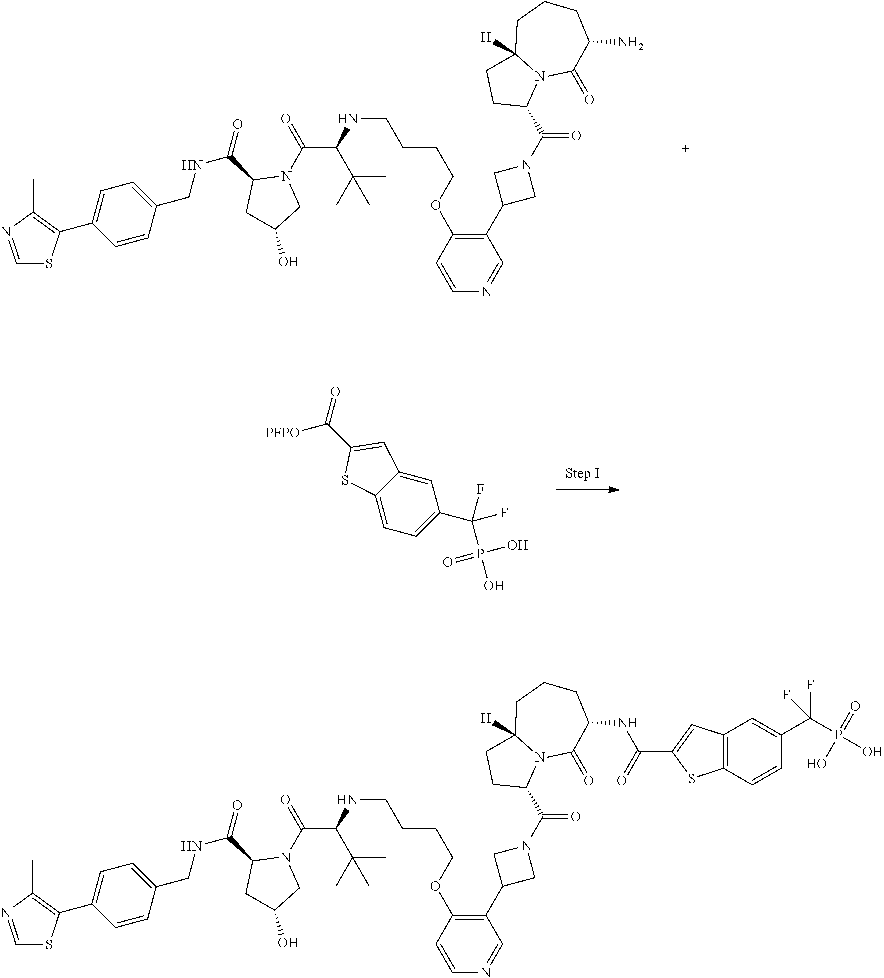

### STEP A: tert-butyl 3-(4-(4-(benzyloxy)butoxy)pyridin-3-yl)azetidine-1-carboxylate

NaH (72 mg, 1.78 mmol, 1.5 eq.) was added to a solution of 4-(benzyloxy)butan-1-ol (257 mg, 1.43 mmol, 1.2 eq.) in DMF (5 mL) at 0° C. and after stirring for 30 min, tert-butyl 3-(4-fluoropyridin-3-yl)azetidine-1-carboxylate (300 mg, 1.19 mmol, 1.0 eq.) was added to the reaction. The reaction mixture was stirred at 25° C. for 10 hrs. After completion, the reaction mixture was quenched with saturated NH₄Cl solution. The residue was partitioned between EtOAc (50 mL×3) and H₂O (50 mL). The combined organic layers were washed with H₂O (50 mL), dried over anhydrous Na₂SO₄, and concentrated under reduced pressure. The residue was purified by flash column chromatography on silica gel to afford tert-butyl 3-(4-(4-(benzyloxy)butoxy)pyridin-3-yl)azetidine-1-carboxylate (400 mg, 0.97 mmol, 82%) as a yellow oil. LCMS (ESI): m/z=413 [M+H]⁺

¹H NMR (400 MHz, CDCl₃) δ 8.38 (d, J=5.6 Hz, 1H), 8.32 (s, 1H), 7.33 (s, 5H), 6.73 (d, J=5.7 Hz, 1H), 4.52 (s, 2H), 4.26 (t, J=8.7 Hz, 2H), 4.14-4.08 (m, 2H), 4.04 (t, J=6.4 Hz, 2H), 3.91 (dd, J=15.7, 7.3 Hz, 1H), 3.54 (t, J=6.1 Hz, 2H), 1.95-1.90 (m, 2H), 1.81-1.76 (m, 2H), 1.45 (s, 9H).

### STEP B: tert-butyl 3-(4-(4-hydroxybutoxy)pyridin-3-yl)azetidine-1-carboxylate

To a solution of tert-butyl 3-(4-(4-(benzyloxy)butoxy)pyridin-3-yl)azetidine-1-carboxylate (400 mg, 0.97 mmol, 1.0 eq.) in MeOH (15 mL) was added Pd/C (40 mg) under nitrogen. The suspension was degassed under vacuum and purged with H₂ several times. The resulting mixture was stirred at room temperature for 4 hrs under H₂ (15 Psi). After completion, the suspension was filtered through a pad of Celite®, the filter cake was washed with MeOH (20 mL). The combined filtrates were concentrated under reduced pressure. The residue was purified by Biotage® C18 column chromatography to give tert-butyl 3-(4-(4-hydroxybutoxy)pyridin-3-yl)azetidine-1-carboxylate (300 mg, 0.93 mmol, 96%) as a yellow oil. LCMS (ESI): m/z=323 [M+H]⁺.

### STEP C: tert-butyl 3-(4-(4-oxobutoxy)pyridin-3-yl)azetidine-1-carboxylate

A mixture of tert-butyl 3-(4-(4-hydroxybutoxy)pyridin-3-yl)azetidine-1-carboxylate (300 mg, 0.93 mmol, 1.0 eq.) and Dess-Martin (790 mg, 1.86 mmol, 2.0 eq.) in dry DCM (10 mL) was stirred at 25° C. for 3 hrs. After completion, the reaction mixture was quenched with Na₂SO₃ (50 mL, sat.) and extracted with DCM (50 mL×3). The combined organic layers were washed with brine (50 mL), dried over anhydrous Na₂SO₄, and concentrated under reduced pressure. The residue was purified by flash column chromatography on silica gel to afford tert-butyl 3-(4-(4-oxobutoxy)pyridin-3-yl)azetidine-1-carboxylate (200 mg, 0.63 mmol, 67%) as a yellow oil. LCMS (ESI): m/z=321 [M+H]⁺.

### STEP D: tert-butyl 3-(4-(4-(((S)-1-((2S,4R)-4-hydroxy-2-((4-(4-methylthiazol-5-yl)benzyl) carbamoyl)pyrrolidin-1-yl)-3,3-dimethyl-1-oxobutan-2-yl)amino)butoxy)pyridin-3-yl)azetidine-1-carboxylate

A mixture of tert-butyl 3-(4-(4-oxobutoxy)pyridin-3-yl)azetidine-1-carboxylate (200 mg, 0.63 mmol, 1.0 eq.) and (2S,4R)-1-((S)-2-amino-3,3-dimethylbutanoyl)-4-hydroxy-N-(4-(4-methylthiazol-5-yl)benzyl)pyrrolidine-2-carboxamide (323 mg, 0.75 mmol, 1.2 eq.) in MeOH (5 mL) was stirred at 25° C. for 2 hrs, Then NaBH₃CN (80 mg, 1.25 mmol, 2.0 eq.) was added at 25° C. and the resulting mixture was stirred at 25° C. for 3 hrs. After completion, the reaction mixture was purified directly by Biotage® C18 column chromatography to afford tert-butyl 3-(4-(4-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)butoxy)pyridin-3-yl)azetidine-1-carboxylate (280 mg, 0.38 mmol, 61%) as a yellow oil. LCMS (ESI): m/z=735 [M+H]⁺.

### STEP E: (2S,4R)-1-((S)-2-((4-((3-(azetidin-3-yl)pyridin-4-yl)oxy)butyl)amino)-3,3-dimethylbutanoyl)-4-hydroxy-N-(4-(4-methylthiazol-5-yl)benzyl)pyrrolidine-2-carboxamide

To a solution of tert-butyl 3-(4-(4-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)butoxy)pyridin-3-yl)azetidine-1-carboxylate (280 mg, 0.38 mmol, 1.0 eq.) in dry DCM (8 mL) was added TFA (2 mL) at 25° C. The mixture was stirred at 25° C. for 2 hrs. After completion, the reaction mixture was concentrated under reduced pressure to give (2S,4R)-1-((S)-2-((4-((3-(azetidin-3-yl)pyridin-4-yl)oxy)butyl)amino)-3,3-dimethylbutanoyl)-4-hydroxy-N-(4-(4-methylthiazol-5-yl)benzyl)pyrrolidine-2-carboxamide (230 mg, quant.) as a yellow oil, which was used in next step without further purification.

LCMS (ESI): m/z=635 [M+H]⁺.

### STEP F: tert-butyl ((3S,6S,9aS)-3-(3-(4-(4-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)butoxy)pyridin-3-yl)azetidine-1-carbonyl)-5-oxooctahydro-1H-pyrrolo[1,2-a]azepin-6-yl)carbamate

To a mixture of (3S,6S,9aS)-6-((tert-butoxycarbonyl)amino)-5-oxooctahydro-1H-pyrrolo[1,2-a]azepine-3-carboxylic acid (136 mg, 0.43 mmol, 1.2 eq.) and HATU (206 mg, 0.54 mmol, 1.5 eq.) in DMF (5 mL) were added DIEA (145 mg, 1.1 mmol, 3.0 eq.) and (2S,4R)-1-((S)-2-((4-((3-(azetidin-3-yl)pyridin-4-yl)oxy)butyl)amino)-3,3-dimethylbutanoyl)-4-hydroxy-N-(4-(4-methylthiazol-5-yl)benzyl)pyrrolidine-2-carboxamide (230 mg, 0.36 mmol, 1.0 eq.). The resulting mixture was stirred at 25° C. for 30 min. After completion, the reaction mixture was purified directly by Biotage® C18 column chromatography to afford tert-butyl ((3S,6S,9aS)-3-(3-(4-(4-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)butoxy)pyridin-3-yl)azetidine-1-carbonyl)-5-oxooctahydro-1H-pyrrolo[1,2-a]azepin-6-yl)carbamate (170 mg, 0.18 mmol, 51%) as a yellow oil. LCMS (ESI): m/z=929.5 [M+H]⁺.

### STEP G: (2S,4R)-1-((S)-2-((4-((3-(1-((3S,6S,9aS)-6-amino-5-oxooctahydro-1H-pyrrolo[1,2-a]azepine-3-carbonyl)azetidin-3-yl)pyridin-4-yl)oxy)butyl)amino)-3,3-dimethylbutanoyl)-4-hydroxy-N-(4-(4-methylthiazol-5-yl)benzyl)pyrrolidine-2-carboxamide

To a solution of tert-butyl ((3S,6S,9aS)-3-(3-(4-(4-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)butoxy)pyridin-3-yl)azetidine-1-carbonyl)-5-oxooctahydro-1H-pyrrolo[1,2-a]azepin-6-yl)carbamate (170 mg, 0.18 mmol, 1.0 eq.) in dry DCM (4 mL) was added TFA (1 mL) at 25° C. The mixture was stirred at 25° C. for 2 hrs. After completion, the reaction mixture was concentrated under reduced pressure to give (2S,4R)-1-((S)-2-((4-((3-(1-((3S,6S,9aS)-6-amino-5-oxooctahydro-1H-pyrrolo[1,2-a]azepine-3-carbonyl)azetidin-3-yl)pyridin-4-yl)oxy)butyl)amino)-3,3-dimethylbutanoyl)-4-hydroxy-N-(4-(4-methylthiazol-5-yl)benzyl)pyrrolidine-2-carboxamide (140 mg, quant.) as a yellow oil, which was used in next step without further purification. LCMS (ESI): m/z=829.4 [M+H]⁺.

### STEP H: S,S((((difluoro(2-(((3S,6S,9aS)-3-(3-(4-(4-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)butoxy)pyridin-3-yl)azetidine-1-carbonyl)-5-oxooctahydro-1H-pyrrolo[1,2-a]azepin-6-yl)carbamoyl)benzo[b]thiophen-5-yl)methyl)phosphoryl)bis(oxy))bis(ethane-2,1-diyl)) dibutanethioate

To a mixture of (2S,4R)-1-((S)-2-((4-((3-(1-((3S,6S,9aS)-6-amino-5-oxooctahydro-1H-pyrrolo[1,2-a]azepine-3-carbonyl)azetidin-3-yl)pyridin-4-yl)oxy)butyl)amino)-3,3-dimethylbutanoyl)-4-hydroxy-N-(4-(4-methylthiazol-5-yl)benzyl)pyrrolidine-2-carboxamide (60 mg, 72 μmol, 1.0 eq.) and DIEA (28 mg, 0.22 mmol, 3.0 eq.) in DMF (3 mL) was added perfluorophenyl 5-((bis(2-(butyrylthio)ethoxy)phosphoryl)difluoromethyl)benzo[b]thiophene-2-carboxylate (58 mg, 79 μmol, 1.1 eq.). The mixture was stirred at 25° C. for 30 min. After completion, the reaction mixture was purified by prep-HPLC to give S,S [symbol] ((((difluoro(2-(((3S,6S,9aS)-3-(3-(4-(4-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)butoxy)pyridin-3-yl)azetidine-1-carbonyl)-5-oxooctahydro-1H-pyrrolo[1,2-a]azepin-6-yl)carbamoyl)benzo[b]thiophen-5-yl)methyl)phosphoryl)bis(oxy))bis(ethane-2,1-diyl)) dibutanethioate (TFA salt) (Compound 1, 35 mg, 25 μmol, 35%) as a yellow oil.

¹H NMR (400 MHz, CDCl₃) δ 9.13 (s, 1H), 8.52 (s, 2H), 8.19-7.82 (m, 5H), 7.64-7.54 (m, 1H), 7.37-7.27 (m, 3H), 5.04-3.81 (m, 20H), 3.63-3.44 (m, 1H), 3.35-3.00 (m, 6H), 2.60-2.43 (m, 7H), 2.39-1.57 (m, 20H), 1.11 (s, 9H), 0.92 (t, J=7.3 Hz, 6H).

LCMS (ESI): m/z=1379.5 [M+H]⁺.

### STEP I: (difluoro(2-(((3S,6S,9aS)-3-(3-(4-(4-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)butoxy)pyridin-3-yl)azetidine-1-carbonyl)-5-oxooctahydro-1H-pyrrolo[,2-a]azepin-6-yl)carbamoyl)benzo[b]thiophen-5-yl)methyl)phosphonic acid

To a mixture of (2S,4R)-1-((S)-2-((4-((3-(1-((3S,6S,9aS)-6-amino-5-oxooctahydro-1H-pyrrolo[1,2-a]azepine-3-carbonyl)azetidin-3-yl)pyridin-4-yl)oxy)butyl)amino)-3,3-dimethylbutanoyl)-4-hydroxy-N-(4-(4-methylthiazol-5-yl)benzyl)pyrrolidine-2-carboxamide (40 mg, 48 μmol, 1.0 eq.) and DIEA (18 mg, 0.14 mmol, 3.0 eq.) in DMF (3 mL) was added (difluoro(2-((perfluorophenoxy)carbonyl)benzo[b]thiophen-5-yl)methyl)phosphonic acid (27 mg, 57 μmol, 1.2 eq.). The mixture was stirred at 25° C. for 30 min. After completion, the reaction mixture was purified by prep-HPLC to give (difluoro(2-(((3S,6S,9aS)-3-(3-(4-(4-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)butoxy)pyridin-3-yl)azetidine-1-carbonyl)-5-oxooctahydro-1H-pyrrolo[1,2-a]azepin-6-yl)carbamoyl)benzo[b]thiophen-5-yl)methyl)phosphonic acid (TFA salt) (Compound 2, 20 mg, 18 μmol, 37%) as a yellow oil.

¹H NMR (400 MHz, DMSO-d6) δ 8.99 (s, 1H), 8.76-8.51 (m, 4H), 8.27-7.99 (m, 3H), 7.65-7.54 (m, 1H), 7.48-7.31 (m, 5H), 4.64-4.56 (m, 2H), 4.48-4.41 (m, 2H), 4.38-4.32 (m, 2H), 4.25-4.21 (m, 2H), 4.05-3.98 (m, 4H), 3.87-3.80 (m, 4H), 3.53-3.46 (m, 2H), 2.95-2.84 (m, 2H), 2.44 (s, 3H), 2.21-2.09 (m, 2H), 2.06-1.56 (m, 15H), 1.17-0.92 (m, 9H).

LCMS (ESI): m/z=1119.4 [M+H]⁺.

### Preparation of trans-((2-(((3S,6S,10aS)-3-(3-cyano-4-(3-(4-(4-(2,6-dioxopiperidin-3-yl)phenoxy)butoxy)phenyl)pyrrolidine-1-carbonyl)-5-oxodecahydropyrrolo[1,2-a]azocin-6-yl)carbamoyl)benzo[b]thiophen-5-yl)difluoromethyl)phosphonic acid (Compound 3) and trans-S,S(((((2-(((3S,6S,10aS)-3-(3-cyano-4-(3-(4-(4-(2,6-dioxopiperidin-3-yl)phenoxy)butoxy)phenyl)pyrrolidine-1-carbonyl)-5-oxodecahydropyrrolo[1,2-a]azocin-6-yl)carbamoyl)benzo[b]thiophen-5-yl)difluoromethyl)phosphoryl)bis(oxy))bis(ethane-2,1-diyl)) dibutanethioate (Compound 4)

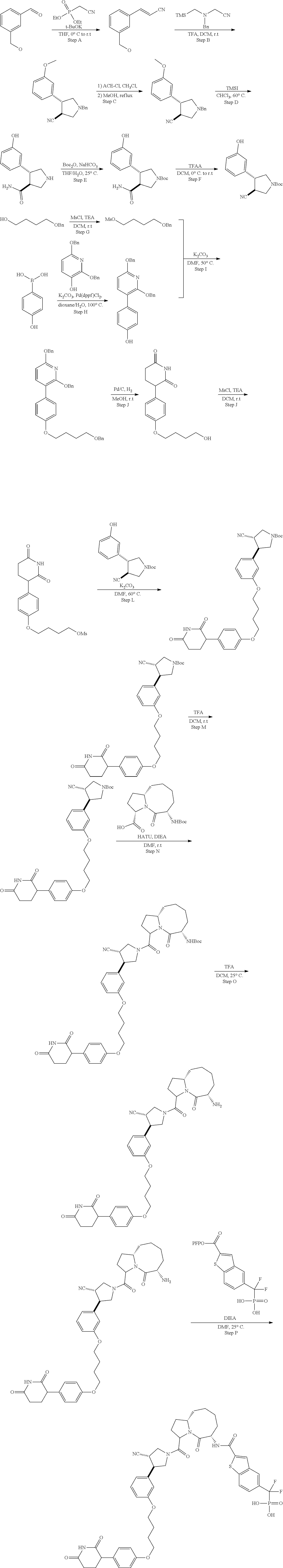

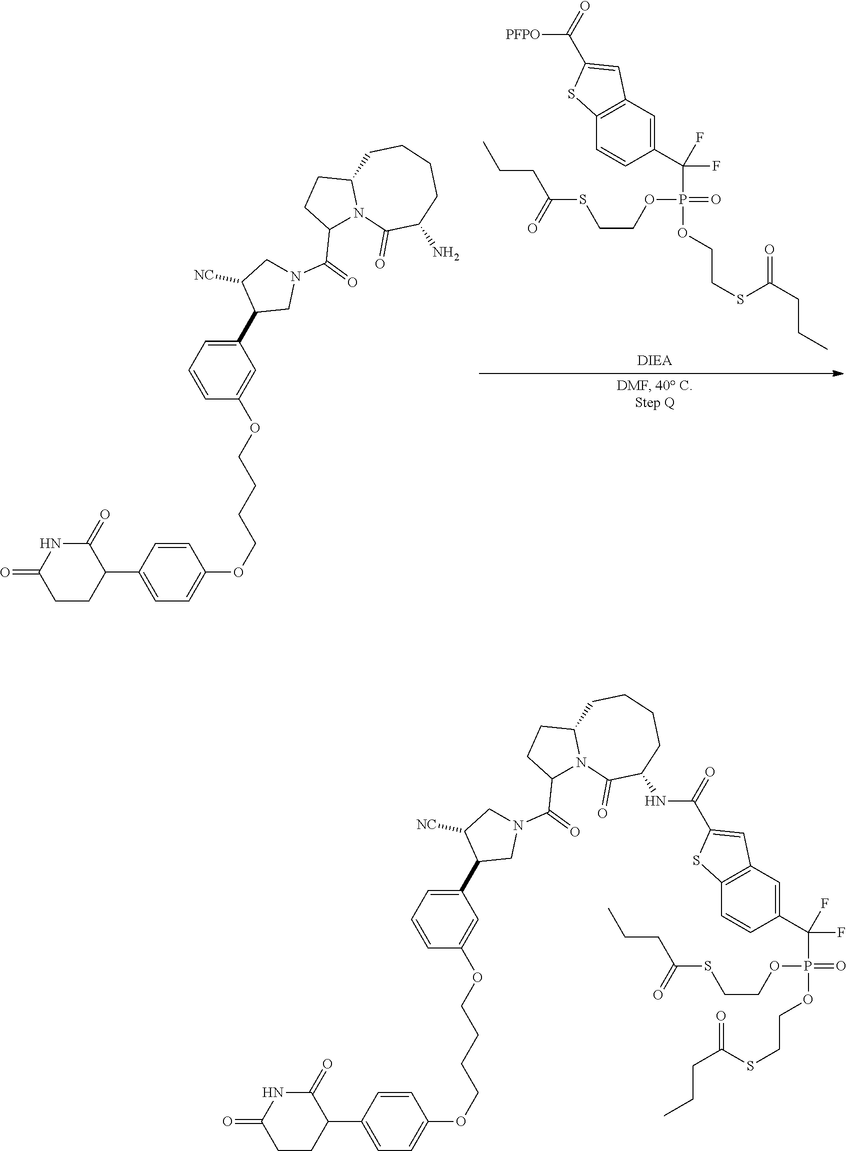

### STEP A: (E)-3-(3-methoxyphenyl)acrylonitrile

To a solution of diethyl (cyanomethyl)phosphonate (13.7 g, 77.2 mmol, 1.05 eq.) in THF (200 mL) was added t-BuOK (9.06 g, 80.9 mmol, 1.1 eq.) at 0° C., the solution was stirred for 30 min, then 3-methoxybenzaldehyde (10 g, 73.5 mmol, 1.0 eq.) was added to the solution. The solution was stirred at room temperature for 30 min. After completion, H₂O (100 mL) was added to the solution, the solution was extracted with EtOAc (200 mL×3). The organic layers were combined and washed with brine (100 mL), dried over anhydrous Na₂SO₄, and concentrated under reduced pressure. The residue was purified by flash column chromatography on silica gel to afford (E)-3-(3-methoxyphenyl)acrylonitrile (11.5 g, 72.3 mmol, 94%) as a white solid. LC-MS (ESI) m/z=160 [M+H]⁺.

### STEP B: trans-1-benzyl-4-(3-methoxyphenyl)pyrrolidine-3-carbonitrile

To a solution of N-benzyl-1-methoxy-N-((trimethylsilyl)methyl)methanamine (16 g, 69.2 mmol, 1.0 eq.) in DCM (10 mL) were added (E)-3-(3-methoxyphenyl)acrylonitrile (11 g, 69.2 mmol, 1.0 eq.) and TFA (1.58 g, 13.8 mmol, 0.2 eq.). The solution was stirred at room temperature overnight. After completion, the reaction mixture was concentrated under reduced pressure. The residue was purified by flash column chromatography on silica gel to afford trans-1-benzyl-4-(3-methoxyphenyl)pyrrolidine-3-carbonitrile (13.1 g, 44.9 mmol, 65%) as a yellow oil. LC-MS (ESI) m/z=293 [M+H]⁺.

### STEP C: trans-4-(3-methoxyphenyl)pyrrolidine-3-carbonitrile

To a solution of trans-1-benzyl-4-(3-methoxyphenyl)pyrrolidine-3-carbonitrile (10.7 g, 36.6 mmol, 1.0 eq.) in CHCl₃ (110 mL) were added ACECl (26.2 g, 183 mmol, 5.0 eq.), the resulting mixture was stirred at 80° C. for 16 hrs. After completion, the reaction mixture concentrated under reduced pressure. The residue was dissolved in MeOH (100 mL) and stirred at 75° C. for 3 hrs. The mixture was concentrated under reduced pressure to afford trans-4-(3-methoxyphenyl)pyrrolidine-3-carbonitrile (7.4 g, crude) as a yellow oil. LCMS (ESI): m/z=203.2 [M+H]⁺.

### STEP D: trans-4-(3-hydroxyphenyl)pyrrolidine-3-carboxamide

To a solution of trans-4-(3-methoxyphenyl)pyrrolidine-3-carbonitrile (7.4 g, 36.6 mmol, 1.0 eq.) in CHCl₃ (80 mL) were added TMSI (22 g, 110 mmol, 3.0 eq.), the resulting mixture was stirred at 60° C. for 16 hrs. After completion, the reaction mixture concentrated under reduced pressure to afford trans-4-(3-hydroxyphenyl)pyrrolidine-3-carboxamide (7.54 g, crude) as a yellow solid. LCMS (ESI): m/z=207.2 [M+H]⁺.

### STEP E: trans-tert-butyl 3-carbamoyl-4-(3-hydroxyphenyl)pyrrolidine-1-carboxylate

To a solution of trans-4-(3-hydroxyphenyl)pyrrolidine-3-carboxamide (7.2 g, 34.9 mmol, 1.0 eq.) and NaHCO₃ (16.1 g, 191.5 mmol, 5.5 eq.) in THF/H₂O (200 mL/40 mL) was added Boc₂O (16.6 g, 76.6 mmol, 2.2 eq.) at 25° C. The resulting mixture was stirred for 16 hrs. After completion, the reaction mixture was filtered and the filtrate was concentrated under reduced pressure. The residue was purified by flash column chromatography on silica gel to afford trans-tert-butyl 3-carbamoyl-4-(3-hydroxyphenyl)pyrrolidine-1-carboxylate (7.0 g, 22.9 mmol, 66%) as a yellow oil. LCMS (ESI): m/z=307.2 [M+H]⁺.

### STEP F: trans-tert-butyl 3-cyano-4-(3-hydroxyphenyl)pyrrolidine-1-carboxylate

To a solution of trans-tert-butyl 3-carbamoyl-4-(3-hydroxyphenyl)pyrrolidine-1-carboxylate (6.8 g, 22.2 mol, 1.0 eq.) in DCM (100 mL) were added TFAA (6.07 g, 28.9 mol, 1.3 eq.) dropwise at 0° C. After addition, the resulting mixture was warmed up to room temperature and stirred for 16 hrs. After completion, the reaction mixture was concentrated under reduced pressure. The residue was purified by C18 column chromatography to afford trans-tert-butyl 3-cyano-4-(3-hydroxyphenyl)pyrrolidine-1-carboxylate (5.0 g, 17.4 mmol, 78%) as a yellow oil. LCMS (ESI): m/z=289.2 [M+H]⁺.

### STEP G: 4-(benzyioxy)butyl methanesulfonate

To a solution of 4-(benzyloxy)butan-1-ol (1.0 g, 5.56 mmol, 1.0 eq.) in DCM (10 mL) were added TEA (842 mg, 8.34 mmol, 1.5 eq.) and MsCl (764 mg, 6.67 mmol, 1.2 eq.) at 0° C. After addition, the resulting mixture was warmed up to room temperature and stirred for 1 hr. After completion, the reaction mixture was diluted with H₂O (10 mL), then extracted with EtOAc (15 mL×3). The organic layers were combined and washed with HCl (1N) and brine (20 mL), dried over anhydrous Na₂SO₄, and concentrated under reduced pressure. The residue was purified by flash column chromatography on silica gel to afford 4-(benzyloxy)butyl methanesulfonate (1.3 g, 5.04 mmol, 91%) as a yellow oil.

¹HNMR (400 MHz, CDCl₃) δ 7.40-7.26 (m, 5H), 4.50 (s, 2H), 4.26 (t, J=6.4 Hz, 2H), 3.51 (t, J=6.1 Hz, 2H), 2.97 (s, 3H), 1.94-1.81 (m, 2H), 1.75-1.71 (m, 2H).

### STEP H: 4-(2,6-bis(benzyloxy)pyridin-3-yl)phenol

To a solution of 2,6-bis(benzyloxy)-3-bromopyridine (10.0 g, 27.1 mmol, 1.0 eq.) and (4-hydroxyphenyl)boronic acid (3.74 g, 27.1 mmol, 1.0 eq.) in dioxane/H₂O (10 mL, v/v=5:1) were added K₂CO₃ (11.2 g, 81.3 mmol, 3.0 eq.) and Pd(dppf)Cl₂ (983 mg, 1.36 mmol, 0.05 eq), and the resulting mixture was stirred at 100° C. for additional 12 hrs. After completion, the reaction mixture was diluted with H₂O (100 mL), extracted with EtOAc (100 mL×3). The organic layers were combined and washed with brine (100 mL), dried over anhydrous Na₂SO₄, and concentrated under reduced pressure. The residue was purified by flash column chromatography on silica gel to afford 4-(2,6-bis(benzyloxy)pyridin-3-yl)phenol (8.0 g, 20.9 mmol, 77%) as a white solid. LCMS (ESI): m/z=384 [M+H]⁺.

### STEP I: 4-(benzyloxy)butyl methanesulfonate

To a solution of 4-(2,6-bis(benzyloxy)pyridin-3-yl)phenol (1.3 g, 5.03 mmol, 1.0 eq.) and K₂CO₃ (1.39 g, 10.1 mmol, 2.0 eq.) in DMF (20 mL) were added 4-(benzyloxy)butyl methanesulfonate (1.95 g, 7.55 mmol, 1.5 eq.) and DMAP (61 mg, 0.50 mmol, 0.1 eq.) at room temperature. After addition, the resulting mixture was heated at 50° C. and stirred for 1 hr. After completion, the reaction mixture was diluted with H₂O (20 mL), then extracted with EtOAc (50 mL×3). The organic layers were combined and washed with brine (20 mL), dried over anhydrous Na₂SO₄, and concentrated under reduced pressure. The residue was purified by flash column chromatography on silica gel to afford 4-(benzyloxy)butyl methanesulfonate (1.98 g, 3.63 mmol, 70%) as a colorless oil.

¹HNMR (400 MHz, CDCl₃) δ 7.57 (d, J=8.0 Hz, 1H), 7.51-7.26 (m, 17H), 6.90 (d, J=8.7 Hz, 2H), 6.45 (d, J=8.0 Hz, 1H), 5.42 (s, 2H), 5.35 (s, 2H), 4.52 (s, 2H), 4.01 (t, J=6.2 Hz, 2H), 3.56 (t, J=6.2 Hz, 2H), 1.95-1.86 (m, 2H), 1.86-1.74 (m, 2H).

### STEP J: 3-(4-(4-hydroxybutoxy)phenyl)piperidine-2,6-dione

To a solution of 4-(benzyloxy)butyl methanesulfonate (1.9 g, 3.49 mmol, 1 eq.) in MeOH (20 mL) was added Pd/C (200 mg) under nitrogen. The suspension was degassed under vacuum and purged with H₂ several times. The resulting mixture was stirred at room temperature for 14 hrs. After completion, the suspension was filtered through a pad of Celite®, the filter cake was washed with MeOH (20 mL). The combined filtrates were concentrated to dryness to give 3-(4-(4-hydroxybutoxy)phenyl)piperidine-2,6-dione (900 mg, 3.25 mmol, 93%) as a white solid. LCMS (ESI): m/z=278.2 (M+H)+.

### STEP K: 4-(4-(2,6-dioxopiperidin-3-yl)phenoxy)butyl methanesulfonate

To a solution of 3-(4-(4-hydroxybutoxy)phenyl)piperidine-2,6-dione (100 mg, 0.36 mmol, 1.0 eq.) in DCM (3 mL) were added TEA (55 mg, 0.54 mmol, 1.5 eq.) and MsCl (50 mg, 0.43 mmol, 1.2 eq.) at 0° C. After addition, the resulting mixture was warmed up to room temperature and stirred for 1 hr. After completion, the reaction mixture was diluted with H₂O (10 mL), then extracted with EtOAc (15 mL×3). The organic layers were combined and washed with brine (10 mL), dried over anhydrous Na₂SO₄, and concentrated under reduced pressure. The residue was purified by flash column chromatography on silica gel to afford 4-(4-(2,6-dioxopiperidin-3-yl)phenoxy)butyl methanesulfonate (116 mg, 0.33 mmol, 91%) as a white solid. LCMS (ESI): m/z=356 (M+H)+.

### STEP L: trans-tert-butyl 3-cyano-4-(3-(4-(4-(2,6-dioxopiperidin-3-yl)phenoxy)butoxy)phenyl)pyrrolidine-1-carboxylate

To a solution of trans-tert-butyl 3-cyano-4-(3-hydroxyphenyl)pyrrolidine-1-carboxylate (94 mg, 0.33 mmol, 1.0 eq.) and K₂CO₃ (91 mg, 0.66 mmol, 2.0 eq.) in DMF (2 mL) were added 4-(4-(2,6-dioxopiperidin-3-yl)phenoxy)butyl methanesulfonate (116 mg, 0.33 mmol, 1.5 eq.) at room temperature. After addition, the resulting mixture was heated at 60° C. and stirred for 1 hr. After completion, the reaction mixture was diluted with H₂O (10 mL), then extracted with EtOAc (10 mL×3). The organic layers were combined and washed with brine (10 mL), dried over anhydrous Na₂SO₄, and concentrated under reduced pressure. The residue was purified by flash column chromatography on silica gel to afford trans-tert-butyl 3-cyano-4-(3-(4-(4-(2,6-dioxopiperidin-3-yl)phenoxy)butoxy)phenyl)pyrrolidine-1-carboxylate (93 mg, 0.17 mmol, 52%) as a colorless oil. LCMS (ESI): m/z=548.3 (M+H)+.

### STEP M: trans-4-(3-(4-(4-(2,6-dioxopiperidin-3-yl)phenoxy)butoxy)phenyl)pyrroidine-3-carbonitrile

To a solution of trans-tert-butyl 3-cyano-4-(3-(4-(4-(2,6-dioxopiperidin-3-yl)phenoxy)butoxy)phenyl)pyrrolidine-1-carboxylate (93 mg, 0.17 mmol, 1.0 eq) in DCM (2 mL) was added TFA (1 mL) at 25° C. The mixture was stirred at room temperature for 0.5 hrs. After completion, the reaction mixture was concentrated under reduced pressure to afford trans-4-(3-(4-(4-(2,6-dioxopiperidin-3-yl)phenoxy)butoxy)phenyl)pyrrolidine-3-carbonitrile (76 mg, quant.) as a yellow oil, which was used in next step without further purification.

LCMS (ESI): m/z=448.2 (M+H)+.

### STEP N: trans-tert-butyl ((3S,6S,10aS)-3-(3-cyano-4-(3-(4-(4-(2,6-dioxopiperidin-3-yl)phenoxy)butoxy)phenyl)pyrrolidine-1-carbonyl)-5-oxodecahydropyrrolo[1,2-a]azocin-6-yl)carbamate

A solution of (3S,6S,10aS)-6-((tert-butoxycarbonyl)amino)-5-oxodecahydropyrrolo[1,2-a]azocine-3-carboxylic acid (55 mg, 0.17 mmol, 1.0 eq.), DIEA (110 mg, 0.85 mmol, 5.0 eq.) and HATU (78 mg, 0.20 mmol, 1.2 eq.) in DMF (2 mL) was stirred for 20 min, then trans-4-(3-(4-(4-(2,6-dioxopiperidin-3-yl)phenoxy)butoxy)phenyl)pyrrolidine-3-carbonitrile (76 mg, 0.17 mmol, 1.0 eq.) were added, and the resulting mixture was stirred at room temperature for additional 2 hrs. After completion, the reaction was purified by C18 column to afford trans-tert-butyl ((3S,6S,10aS)-3-(3-cyano-4-(3-(4-(4-(2,6-dioxopiperidin-3-yl)phenoxy)butoxy)phenyl)pyrrolidine-1-carbonyl)-5-oxodecahydropyrrolo[1,2-a]azocin-6-yl)carbamate (108 mg, 0.14 mmol, 82%) as a white solid. LCMS (ESI): m/z=756.4 [M+H]⁺.

### STEP O: trans-1-((3S,6S,10aS)-6-amino-5-oxodecahydropyrrolo[1,2-a]azocine-3-carbonyl)-4-(3-(4-(4-(2,6-dioxopiperidin-3-yl)phenoxy)butoxy)phenyl)pyrrolidine-3-carbonitrile

To a solution of trans-tert-butyl ((3S,6S,10aS)-3-(3-cyano-4-(3-(4-(4-(2,6-dioxopiperidin-3-yl)phenoxy)butoxy)phenyl)pyrrolidine-1-carbonyl)-5-oxodecahydropyrrolo[1,2-a]azocin-6-yl)carbamate (100 mg, 0.132 mmol, 1.0 eq) in DCM (3 mL) was added TFA (1 mL) at 25° C. The mixture was stirred at 25° C. for 0.5 hrs. LCMS indicated the complete consumption of the starting material. The solution was concentrated under reduce pressure to give trans-1-((3S,6S,10aS)-6-amino-5-oxodecahydropyrrolo[1,2-a]azocine-3-carbonyl)-4-(3-(4-(4-(2,6-dioxopiperidin-3-yl)phenoxy)butoxy)phenyl)pyrrolidine-3-carbonitrile (100 mg, crude) as a yellow oil. LCMS (ESI): m/z=656 [M+H]⁺.

### STEP P: trans-((2-(((3S,6S,10aS)-3-(3-cyano-4-(3-(4-(4-(2,6-dioxopiperidin-3-yl)phenoxy)butoxy)phenyl)pyrrolidine-1-carbonyl)-5-oxodecahydropyrrolo[1,2-a]azocin-6-yl)carbamoyl)benzo[b]thiophen-5-yl)difluoromethyl)phosphonic acid

To a solution of trans-1-((3S,6S,10aS)-6-amino-5-oxodecahydropyrrolo[1,2-a]azocine-3-carbonyl)-4-(3-(4-(4-(2,6-dioxopiperidin-3-yl)phenoxy)butoxy)phenyl)pyrrolidine-3-carbonitrile (50 mg, 75 μmol, 1.0 eq.) in DMF (1 mL) were added DIEA (98 mg, 0.76 mmol, 10 eq.) and (difluoro(2-((perfluorophenoxy)carbonyl)benzo[b]thiophen-5-yl)methyl)phosphonic acid (32 mg, 68 μmol, 0.9 eq.) at 25° C. The reaction mixture was stirred at 25° C. for 0.5 hrs. After completion, the reaction mixture was purified by prep-HPLC to afford trans-((2-(((3S,6S,10aS)-3-(3-cyano-4-(3-(4-(4-(2,6-dioxopiperidin-3-yl)phenoxy)butoxy)phenyl)pyrrolidine-1-carbonyl)-5-oxodecahydropyrrolo[1,2-a]azocin-6-yl)carbamoyl)benzo[b]thiophen-5-yl)difluoromethyl)phosphonic acid (Compound 3, 15 mg, 16 μmol, 24%) as a white solid.

¹H NMR (400 MHz, DMSO-d6) δ 10.89-10.66 (m, 1H), 8.91-8.78 (m, 1H), 8.37-8.28 (m, 1H), 8.18-8.03 (m, 2H), 7.64-7.54 (m, 1H), 7.33-7.23 (m, 1H), 7.15-7.08 (m, 2H), 7.07-6.94 (m, 2H), 6.93-6.83 (m, 3H), 4.98-4.91 (m, 1H), 4.59-4.53 (m, 1H), 4.35-4.21 (m, 2H), 4.02-3.88 (m, 3H), 3.84-3.73 (m, 2H), 3.71-3.60 (m, 1H), 3.57-3.46 (m, 1H), 3.34-3.22 (m, 1H), 3.16-3.01 (m, 1H), 2.71-2.59 (m, 1H), 2.35-2.12 (m, 2H), 2.07-1.71 (m, 13H), 1.69-1.45 (m, 3H), 1.30-1.12 (m, 2H).

LCMS (ESI): m/z=946 [M+H]⁺.

### STEP Q: trans-S,S((((2-(((3S,6S,10aS)-3-(3-cyano-4-(3-(4-(4-(2,6-dioxopiperidin-3-yl)phenoxy)butoxy)phenyl)pyrrolidine-1-carbonyl)-5-oxodecahydropyrrolo[1,2-a]azocin-6-yl)carbamoyl)benzo[b]thiophen-5-yl)difluoromethyl)phosphoryl)bis(oxy))bis(ethane-2,1-diyl)) dibutanethioate

To a solution of trans-1-((3S,6S,10aS)-6-amino-5-oxodecahydropyrrolo[1,2-a]azocine-3-carbonyl)-4-(3-(4-(4-(2,6-dioxopiperidin-3-yl)phenoxy)butoxy)phenyl)pyrrolidine-3-carbonitrile (50 mg, 75 μmol, 1.0 eq.) in DMF (1 mL) were added DIEA (98 mg, 0.76 mmol, 10 eq.) and 4-nitrophenyl 5-((bis(2-(butyrylthio)ethoxy)phosphoryl)difluoromethyl)benzo[b]thiophene-2-carboxylate (45 mg, 65 μmol, 0.87 eq.) at 25° C. The reaction mixture was heated to 40° C. and stirred at 40° C. for 12 hrs. After completion, the reaction mixture was purified by prep-HPLC to afford trans-S,S [symbol] (((((2-(((3S,6S,10aS)-3-(3-cyano-4-(3-(4-(4-(2,6-dioxopiperidin-3-yl)phenoxy)butoxy)phenyl)pyrrolidine-1-carbonyl)-5-oxodecahydropyrrolo[1,2-a]azocin-6-yl)carbamoyl)benzo[b]thiophen-5-yl)difluoromethyl)phosphoryl)bis(oxy))bis(ethane-2,1-diyl)) dibutanethioate (Compound 4, 15 mg, 15 μmol, 24%) as a white solid.

¹H NMR (400 MHz, DMSO-d6) δ 10.85-10.72 (m, 1H), 8.99-8.85 (m, 1H), 8.40-8.30 (m, 1H), 8.27-8.11 (m, 2H), 7.63-7.54 (m, 1H), 7.33-7.24 (m, 1H), 7.16-7.07 (m, 2H), 7.07-6.93 (m, 2H), 6.93-6.82 (m, 3H), 5.06-4.89 (m, 1H), 4.64-4.46 (m, 1H), 4.41-3.44 (m, 16H), 3.20-3.07 (m, 4H), 2.71-2.58 (m, 1H), 2.58-2.52 (m, 4H), 2.36-1.42 (m, 22H), 1.34-1.14 (m, 1H), 0.93-0.78 (m, 6H).

LCMS (ESI): m/z=1206 [M+H]⁺.

### Preparation of ((2-(((3S,6S,10aS)-3-((6R)-6-(1-(8-(2-(2,6-dioxopiperidin-3-yl)-1-oxoisoindolin-4-yl)oct-7-yn-1-yl)-2-oxo-1,2-dihydropyridin-4-yl)-4-azaspiro[2.4]heptane-4-carbonyl)-5 oxodecahydropyrrolo[1,2-a]azocin-6-yl)carbamoyl)benzo[b]thiophen-5-yl)difluoromethyl)phosphonic acid (Compound 5) and S,S(((((2-(((3S,6S,10aS)-3-((6R)-6-(1-(8-(2-(2,6-dioxopiperidin-3-yl)-1-oxoisoindolin-4-yl)oct-7-yn-1-yl)-2-oxo-1,2-dihydropyridin-4-yl)-4-azaspiro[2.4]heptane-4-carbonyl)-5-oxodecahydropyrrolo[1,2-a]azocin-6-yl)carbamoyl)benzo[b]thiophen-5-yl)difluoromethyl)phosphoryl)bis(oxy))bis(ethane-2,1-diyl)) dibutanethioate (Compound 5)

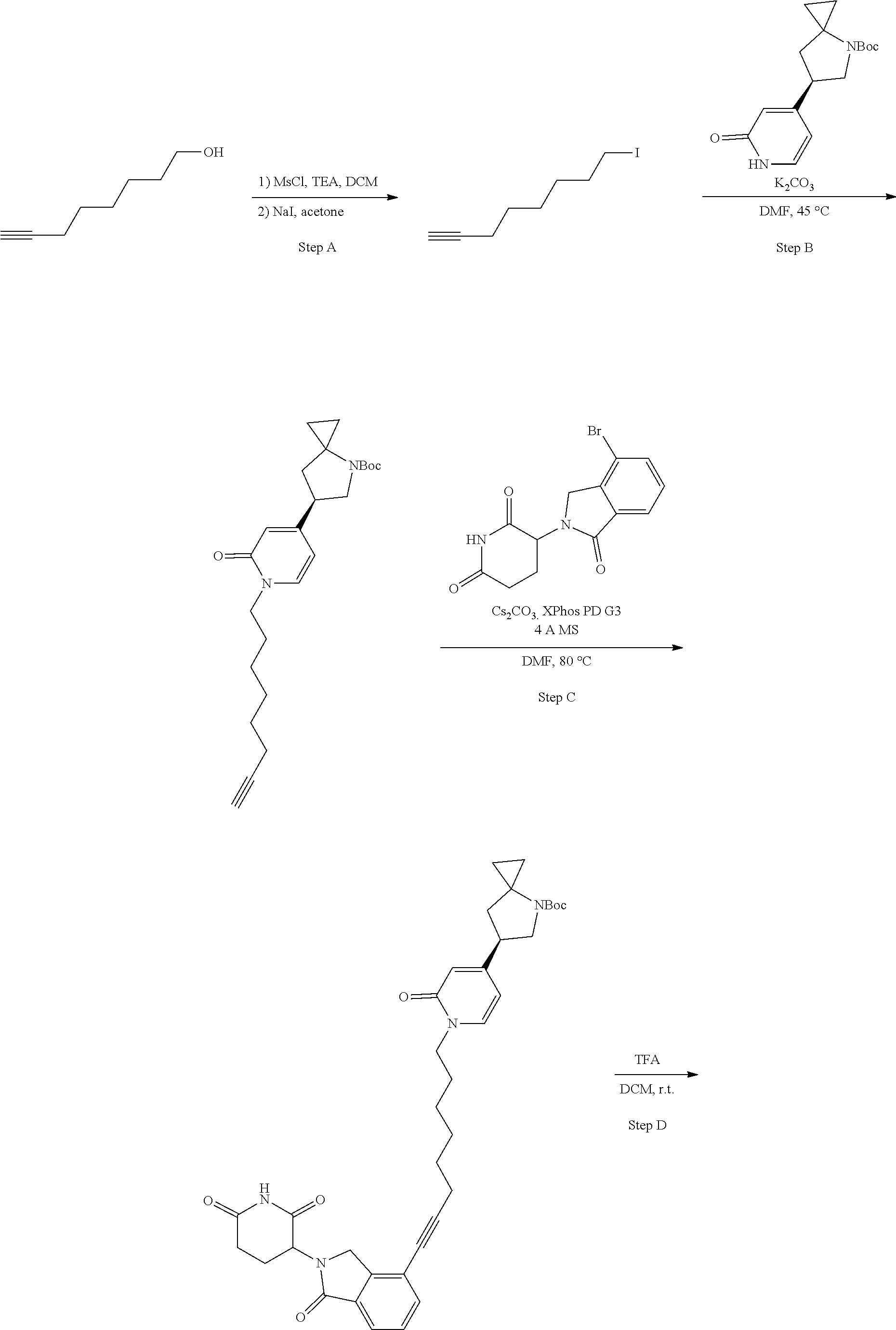

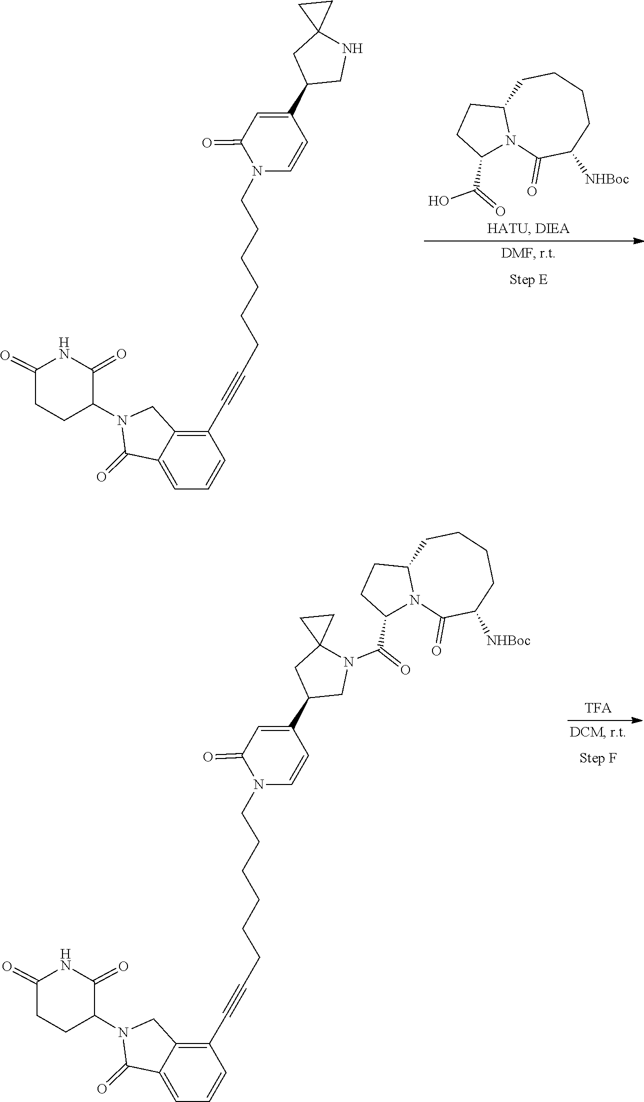 [SMILES: O=C1CCC(N2Cc3c(C#CCCCCCCn4ccc([C@@H]5CNC6(CC6)C5)cc4=O)cccc3C2=O)C(=O)N1 ; CC(C)(C)OC(=O)N[C@H]1CCCC[C@H]2CC[C@@H](C(=O)O)N2C1=O ; CC(C)(C)OC(=O)N[C@H]1CCCC[C@H]2CC[C@@H](C(=O)N3C[C@@H](c4ccn(CCCCCCC#Cc5cccc6c5CN(C5CCC(=O)NC5=O)C6=O)c(=O)c4)CC34CC4)N2C1=O]

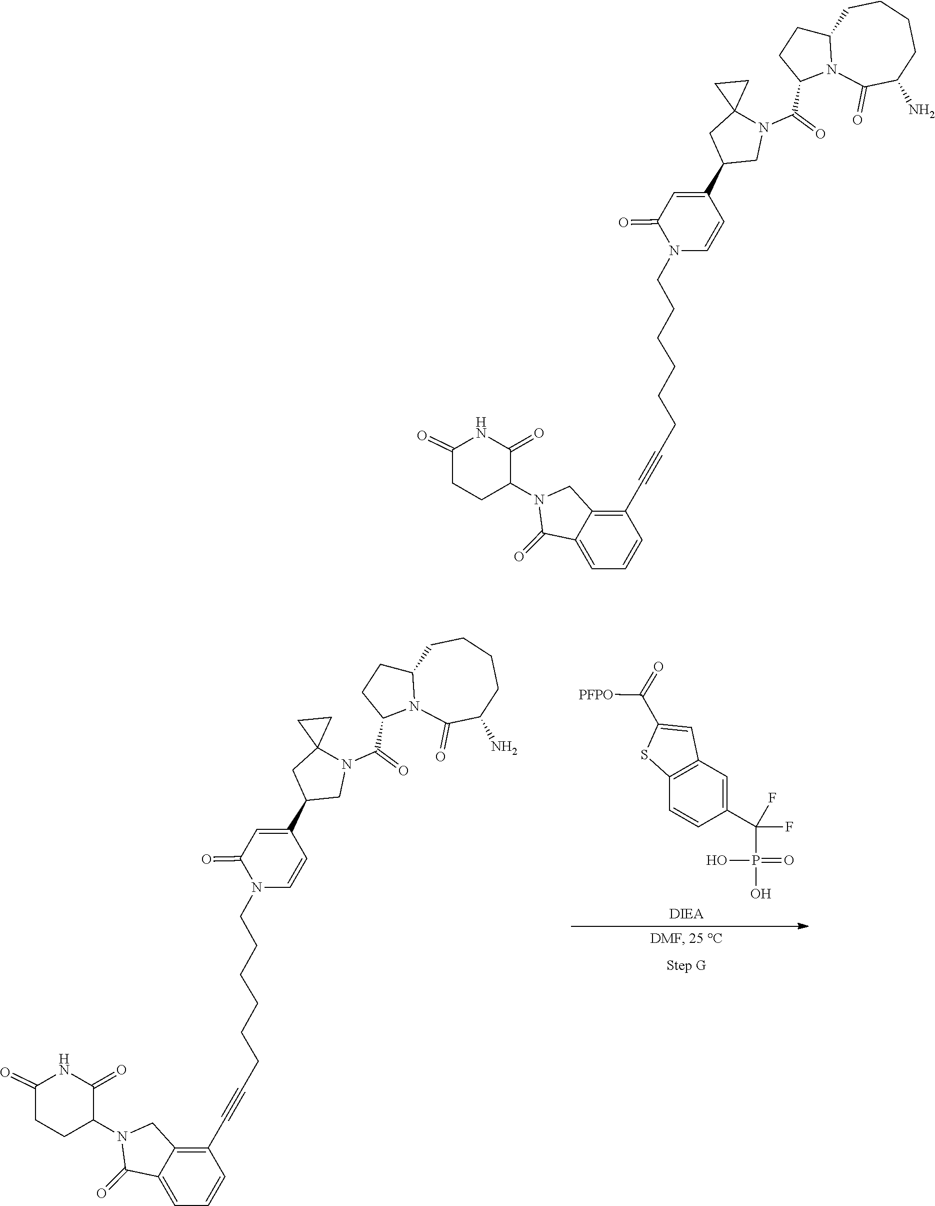

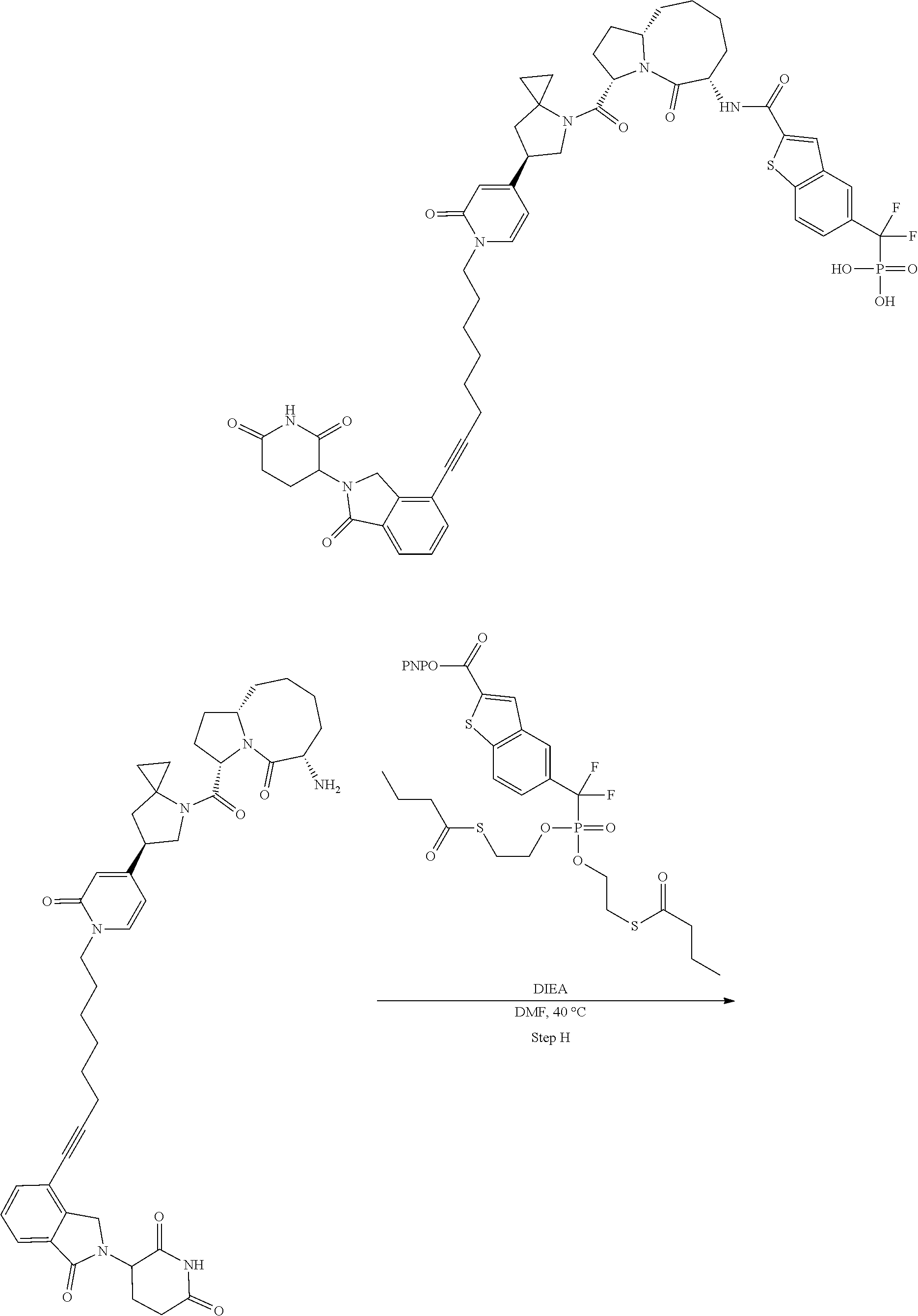

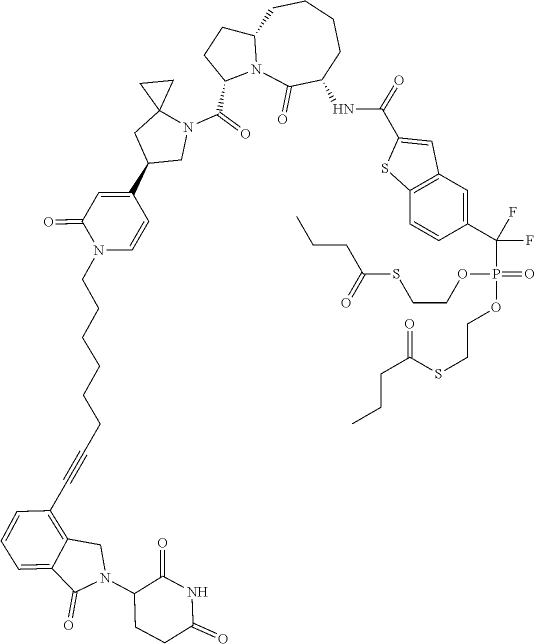 [SMILES: CCCC(=O)SCCOP(=O)(OCCSC(=O)CCC)C(F)(F)c1ccc2sc(C(=O)N[C@H]3CCCC[C@H]4CC[C@@H](C(=O)N5C[C@@H](c6ccn(CCCCCCC#Cc7cccc8c7CN(C7CCC(=O)NC7=O)C8=O)c(=O)c6)CC56CC6)N4C3=O)cc2c1]

### STEP A: 8-Iodooct-1-yne

To a solution of oct-7-yn-1-ol (2 g, 15.8 mmol, 1.0 eq.) in DCM (15 mL) were added TEA (3.21 g, 31.6 mmol, 2.0 eq.) and MsCl (2.18 g, 19.0 mmol, 1.2 eq.) at 0° C. The reaction mixture was stirred at room temperature for 1 hr. After completion, the reaction mixture was diluted with H₂O (30 mL) and extracted with DCM (30 mL×2). The combined organic layers were washed with brine (20 mL), dried over anhydrous Na₂SO₄ and concentrated under reduced pressure to afford oct-7-yn-1-yl methanesulfonate (4.86 g, 15.8 mmol, 99%). To a solution of oct-7-yn-1-yl methanesulfonate (4.86 g, 15.8 mmol, 1.0 eq.) in 2-butanone (30 mL) was added NaI (7.14 g, 47.4 mmol, 3.0 eq.). The reaction mixture was stirred at 85° C. for 12 hrs. After completion, the reaction mixture was quenched with Na₂SO₃ (40 mL, saturated aqueous solution) and extracted with EtOAc (30 mL×2). The combined organic layers were washed with brine (20 mL), dried over anhydrous Na₂SO₄, and concentrated under reduced pressure. The residue was purified by flash column chromatography on silica gel to afford 8-iodooct-1-yne (3.1 g, 13.1 mmol, 83%) as a light oil.

¹H NMR (400 MHz, DMSO-d6) δ 3.30-3.23 (m, 2H), 2.76-2.67 (m, 1H), 2.18-2.10 (m, 2H), 1.81-1.69 (m, 2H), 1.47-1.31 (m, 6H).

### STEP B: tert-butyl (R)-6-(1-(oct-7-yn-1-yl)-2-oxo-1,2-dihydropyridin-4-yl)-4-azaspiro[2.4]heptane-4-carboxylate

To a solution of 8-iodooct-1-yne (245 mg, 1.04 mmol, 1.0 eq.) in DMF (4 mL) were added tert-butyl (R)-6-(2-oxo-1,2-dihydropyridin-4-yl)-4-azaspiro[2.4]heptane-4-carboxylate (300 mg, 1.04 mmol, 1.0 eq.) and K₂CO₃ (431 mg, 3.12 mmol, 3.0 eq.). The reaction mixture was stirred at 45° C. for 12 hrs. After completion, the reaction mixture was purified directly by Biotage® C18 column chromatography to afford tert-butyl (R)-6-(1-(oct-7-yn-1-yl)-2-oxo-1,2-dihydropyridin-4-yl)-4-azaspiro[2.4]heptane-4-carboxylate (260 mg, 0.65 mmol, 63%) as a light oil. LCMS (ESI): m/z=399 [M+H]⁺.

¹H NMR (400 MHz, DMSO-d6) δ 7.61 (d, J=7.0 Hz, 1H), 6.34-6.15 (m, 2H), 3.92-3.76 (m, 3H), 3.35-3.24 (m, 2H), 2.81-2.68 (m, 1H), 2.28-2.08 (m, 3H), 2.03-1.89 (m, 1H), 1.63-1.54 (m, 2H), 1.49-1.20 (m, 17H), 0.50 (s, 2H).

### STEP C: tert-butyl (6R)-6-(1-(8-(2-(2,6-dioxopiperidin-3-yl)-1-oxoisoindolin-4-yl)oct-7-yn-1-yl)-2-oxo-1,2-dihydropyridin-4-yl)-4-azaspiro[2.4]heptane-4-carboxylate

To a solution of tert-butyl tert-butyl (R)-6-(1-(oct-7-yn-1-yl)-2-oxo-1,2-dihydropyridin-4-yl)-4-azaspiro[2.4]heptane-4-carboxylate (257 mg, 0.65 mmol, 1.1 eq.), 3-(4-bromo-1-oxoisoindolin-2-yl)piperidine-2,6-dione (190 mg, 0.59 mmol, 1.0 eq.), Cs₂CO₃ (575 mg, 1.76 mmol, 3.0 eq.) and 4A molecular sieve (100 mg) in DMF (15 mL) were added CuI (22 mg, 0.12 mmol, 0.2 eq.) and Pd(dppf)Cl₂ (43 mg, 59 μmol, 0.1 eq.). And the reaction mixture was stirred at 80° C. overnight under N₂. The reaction mixture was diluted with H₂O (20 mL) and extracted with EtOAc (20 mL×2). The combined organic layers were washed with brine (10 mL), dried over anhydrous Na₂SO₄, and concentrated under reduced pressure. The residue was purified by C18 column chromatography to give tert-butyl (6R)-6-(1-(8-(2-(2,6-dioxopiperidin-3-yl)-1-oxoisoindolin-4-yl)oct-7-yn-1-yl)-2-oxo-1,2-dihydropyridin-4-yl)-4-azaspiro[2.4]heptane-4-carboxylate (160 mg, 0.25 mmol, 42%) as a yellow solid.

LCMS (ESI): m/z=641 [M+H]⁺.

### STEP D: 3-(I-oxo-4-(8-(2-oxo-4-((R)-4-azaspiro[2.4]heptan-6-yl)pyridin-1(2H)-yl)oct-1-yn-1-yl)isoindolin-2-yl)piperidine-2,6-dione

To a solution of tert-butyl (6R)-6-(1-(8-(2-(2,6-dioxopiperidin-3-yl)-1-oxoisoindolin-4-yl)oct-7-yn-1-yl)-2-oxo-1,2-dihydropyridin-4-yl)-4-azaspiro[2.4]heptane-4-carboxylate (160 mg, 0.25 mmol, 1.0 eq.) in DCM (5 mL) was added TFA (2 mL) and the reaction mixture was stirred at room temperature for 2 hrs under N₂. After completion, the reaction mixture was concentrated under reduced pressure to get crude 3-(1-oxo-4-(8-(2-oxo-4-((R)-4-azaspiro[2.4]heptan-6-yl)pyridin-1(2H)-yl)oct-1-yn-1-yl)isoindolin-2-yl)piperidine-2,6-dione (160 mg, quant.) as a yellow oil, which was used in next step directly without further purification. LCMS (ESI): m/z=541 [M+H]⁺.

### STEP E: tert-butyl ((3S,6S,10aS)-3-((6R)-6-(1-(8-(2-(2,6-dioxopiperidin-3-yl)-1-oxoisoindolin-4-yl)oct-7-yn-1-yl)-2-oxo-1,2-dihydropyridin-4-yl)-4-azaspiro[2.4]heptane-4-carbonyl)-5-oxodecahydropyrrolo[1,2-a]azocin-6-yl)carbamate

To a solution of (3S,6S,10aS)-6-((tert-butoxycarbonyl)amino)-5-oxodecahydropyrrolo[1,2-a]azocine-3-carboxylic acid (96 mg, 0.29 mmol, 1.2 eq.) and HATU (139 mg, 0.37 mmol, 1.5 eq.) in DMF (1 mL) were added 3-(1-oxo-4-(8-(2-oxo-4-((R)-4-azaspiro[2.4]heptan-6-yl)pyridin-1(2H)-yl)oct-1-yn-1-yl)isoindolin-2-yl)piperidine-2,6-dione (160 mg, 0.24 mmol, 1.0 eq.) and DIEA (95 mg, 0.73 mmol, 3 eq.). The resulting mixture was stirred at room temperature for 2 hrs under N₂. After completion, the reaction mixture was purified by Biotage® C18 column chromatography to get tert-butyl ((3S,6S,10aS)-3-((6R)-6-(1-(8-(2-(2,6-dioxopiperidin-3-yl)-1-oxoisoindolin-4-yl)oct-7-yn-1-yl)-2-oxo-1,2-dihydropyridin-4-yl)-4-azaspiro[2.4]heptane-4-carbonyl)-5-oxodecahydropyrrolo[1,2-a]azocin-6-yl)carbamate (130 mg, 0.15 mmol, 56%) as a white solid. LCMS (ESI): m/z=849 [M+H]⁺.

### STEP F: 3-(4-(8-(4-((R)-4-((3S,6S,10aS)-6-amino-5-oxodecahydropyrrolo[1,2-a]azocine-3-carbonyl)-4-azaspiro[2.4]heptan-6-yl)-2-oxopyridin-1(2H)-yl)oct-1-yn-1-yl)-1-oxoisoindolin-2-yl)piperidine-2,6-dione

To a solution of tert-butyl ((3S,6S,10aS)-3-((6R)-6-(1-(8-(2-(2,6-dioxopiperidin-3-yl)-1-oxoisoindolin-4-yl)oct-7-yn-1-yl)-2-oxo-1,2-dihydropyridin-4-yl)-4-azaspiro[2.4]heptane-4-carbonyl)-5-oxodecahydropyrrolo[1,2-a]azocin-6-yl)carbamate (130 mg, 0.15 mmol, 1.0 eq.) in DCM (5 mL) was added TFA (2 mL) and the reaction mixture was stirred at room temperature for 2 hrs under N₂. After completion, the reaction mixture was concentrated under reduced pressure to get crude 3-(4-(8-(4-((R)-4-((3S,6S,10aS)-6-amino-5-oxodecahydropyrrolo[1,2-a]azocine-3-carbonyl)-4-azaspiro[2.4]heptan-6-yl)-2-oxopyridin-1(2H)-yl)oct-1-yn-1-yl)-1-oxoisoindolin-2-yl)piperidine-2,6-dione (120 mg, quant.) as a yellow oil, which was used in next step directly without further purification.

LCMS (ESI): m/z=749 [M+H]⁺.

### STEP G: ((2-(((3S,6S,10aS)-3-((6R)-6-(1-(8-(2-(2,6-dioxopiperidin-3-yl)-1-oxoisoindolin-4-yl)oct-7-yn-1-yl)-2-oxo-1,2-dihydropyridin-4-yl)-4-azaspiro[2.4]heptane-4-carbonyl)-5 oxodecahydropyrrolo[1,2-a]azocin-6-yl)carbamoyl)benzo[b]thiophen-5-yl)difluoromethyl)phosphonic acid

To a solution of 3-(4-(8-(4-((R)-4-((3 S,6S,10aS)-6-amino-5-oxodecahydropyrrolo[1,2-a]azocine-3-carbonyl)-4-azaspiro[2.4]heptan-6-yl)-2-oxopyridin-1(2H)-yl)oct-1-yn-1-yl)-1-oxoisoindolin-2-yl)piperidine-2,6-dione (50 mg, 67 μmol, 1.0 eq.) in DMF (1 mL) were added (difluoro(2-((perfluorophenoxy)carbonyl)benzo[b]thiophen-5-yl)methyl)phosphonic acid (32 mg, 67 μmol, 1.0 eq.) and TEA (20 mg, 200 μmol, 3.0 eq.). The resulting mixture was stirred at 40° C. for 2 hrs under N₂. After completion, the reaction mixture was concentrated under reduced pressure. The residue was purified by prep-HPLC to get ((2-(((3S,6S,10aS)-3-((6R)-6-(1-(8-(2-(2,6-dioxopiperidin-3-yl)-1-oxoisoindolin-4-yl)oct-7-yn-1-yl)-2-oxo-1,2-dihydropyridin-4-yl)-4-azaspiro[2.4]heptane-4-carbonyl)-5 oxodecahydropyrrolo[1,2-a]azocin-6-yl)carbamoyl)benzo[b]thiophen-5-yl)difluoromethyl)phosphonic acid (Compound 5, 16.5 mg, 16 μmol, 24%) as a white solid.

¹H NMR (400 MHz, DMSO-d6) δ 10.98 (s, 1H), 8.85 (d, J=7.1 Hz, 1H), 8.30 (s, 1H), 8.11-8.00 (m, 2H), 7.69 (d, J=7.5 Hz, 1H), 7.62-7.58 (m, 3H), 7.50 (t, J=7.6 Hz, 1H), 6.30 (s, 1H), 6.22-6.20 (m, 1H), 5.14-5.10 (m, 1H), 5.00-4.88 (m, 1H), 4.51-4.41 (m, 2H), 4.32-4.28 (m, 1H), 4.25-4.20 (m, 1H), 4.05 (t, J=8.7 Hz, 1H), 3.80 (t, J=7.1 Hz, 2H), 3.72-3.67 (m, 1H), 3.10-3.03 (m, 1H), 2.94-2.85 (m, 1H), 2.67-2.65 (m, 1H), 2.46-2.42 (m, 3H), 2.34-2.21 (m, 2H), 2.14-2.08 (m, 1H), 2.04-1.96 (m, 4H), 1.89-1.79 (m, 4H), 1.75-1.70 (m, 2H), 1.60-1.51 (m, 8H), 1.45-1.40 (m, 2H), 1.28-1.23 (m, 3H), 1.18-1.14 (m, 1H), 0.50-0.43 (m, 2H).

LCMS (ESI): m/z=1039.3 [M+H]⁺.

### STEP H: S,S((((2-(((3S,6S,10aS)-3-((6R)-6-(1-(8-(2-(2,6-dioxopiperidin-3-yl)-1-oxoisoindolin-4-yl)oct-7-yn-1-yl)-2-oxo-1,2-dihydropyridin-4-yl)-4-azaspiro[2.4]heptane-4-carbonyl)-5-oxodecahydropyrrolo[1,2-a]azocin-6-yl)carbamoyl)benzo[b]thiophen-5-yl)difluoromethyl)phosphoryl)bis(oxy))bis(ethane-2,1-diyl)) dibutanethioate

To a solution of 3-(4-(8-(4-((R)-4-((3S,6S,10aS)-6-amino-5-oxodecahydropyrrolo[1,2-a]azocine-3-carbonyl)-4-azaspiro[2.4]heptan-6-yl)-2-oxopyridin-1(2H)-yl)oct-1-yn-1-yl)-1-oxoisoindolin-2-yl)piperidine-2,6-dione (70 mg, 93 μmol, 1.0 eq.) in DMF (1 mL) were added 4-nitrophenyl 5-((bis(2-(butyrylthio)ethoxy)phosphoryl)difluoromethyl)benzo[b]thiophene-2-carboxylate (64 mg, 93 μmol, 1.0 eq.) and TEA (28 mg, 280 μmol, 3.0 eq.). The resulting mixture was stirred at room temperature for 2 hrs under N₂. After completion, the reaction mixture was purified by prep-HPLC to get S,S [symbol] (((((2-(((3S,6S,10aS)-3-((6R)-6-(1-(8-(2-(2,6-dioxopiperidin-3-yl)-1-oxoisoindolin-4-yl)oct-7-yn-1-yl)-2-oxo-1,2-dihydropyridin-4-yl)-4-azaspiro[2.4]heptane-4-carbonyl)-5-oxodecahydropyrrolo[1,2-a]azocin-6-yl)carbamoyl)benzo[b]thiophen-5-yl)difluoromethyl)phosphoryl)bis(oxy))bis(ethane-2,1-diyl)) dibutanethioate (Compound 6, 36.6 mg, 28 μmol, 30%) as a white solid.

¹H NMR (400 MHz, DMSO-d6) δ 10.97 (s, 1H), 8.93 (d, J=7.1 Hz, 1H), 8.34 (s, 1H), 8.19 (d, J=8.7 Hz, 1H), 8.14 (s, 1H), 7.69 (d, J=7.6 Hz, 1H), 7.62-7.57 (m, 3H), 7.50 (t, J=7.6 Hz, 1H), 6.30 (s, 1H), 6.21 (d, J=6.9 Hz, 1H), 5.15-5.10 (m, 1H), 5.01-4.88 (m, 1H), 4.50-4.46 (m, 2H), 4.32-4.28 (m, 1H), 4.25-4.10 (m, 5H), 4.08-4.04 (m, 1H), 3.82-3.67 (m, 3H), 3.13 (t, J=6.2 Hz, 4H), 2.95-2.83 (m, 1H), 2.62-2.52 (m, 4H), 2.48-2.39 (m, 4H), 2.33-2.24 (m, 1H), 2.14-1.94 (m, 6H), 1.89-1.68 (m, 6H), 1.60-1.50 (m, 12H), 1.46-1.38 (m, 2H), 1.30-1.20 (m, 3H), 0.85 (t, J=7.4 Hz, 6H), 0.50-0.42 (m, 2H).

LCMS (ESI): m/z=1300.2 [M+H]⁺.

### Preparation of S,S((((difluoro(2-(((3S,6S,9aS)-3-(3-(4-(4-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)carbamoyl)piperidin-1-yl)pyridin-3-yl)azetidine-1-carbonyl)-5-oxooctahydro-1H-pyrrolo[1,2-a]azepin-6-yl)carbamoyl)benzo[b]thiophen-5-yl)methyl)phosphoryl)bis(oxy))bis(ethane-2,1-diyl)) dibutanethioate (Compound 7) and (difluoro(2-(((3S,6S,9aS)-3-(3-(4-(4-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)carbamoyl)piperidin-1-yl)pyridin-3-yl)azetidine-1-carbonyl)-5-oxooctahydro-1H-pyrrolo[1,2-a]azepin-6-yl)carbamoyl)benzo[b]thiophen-5-yl)methyl)phosphonic acid (Compound 8)

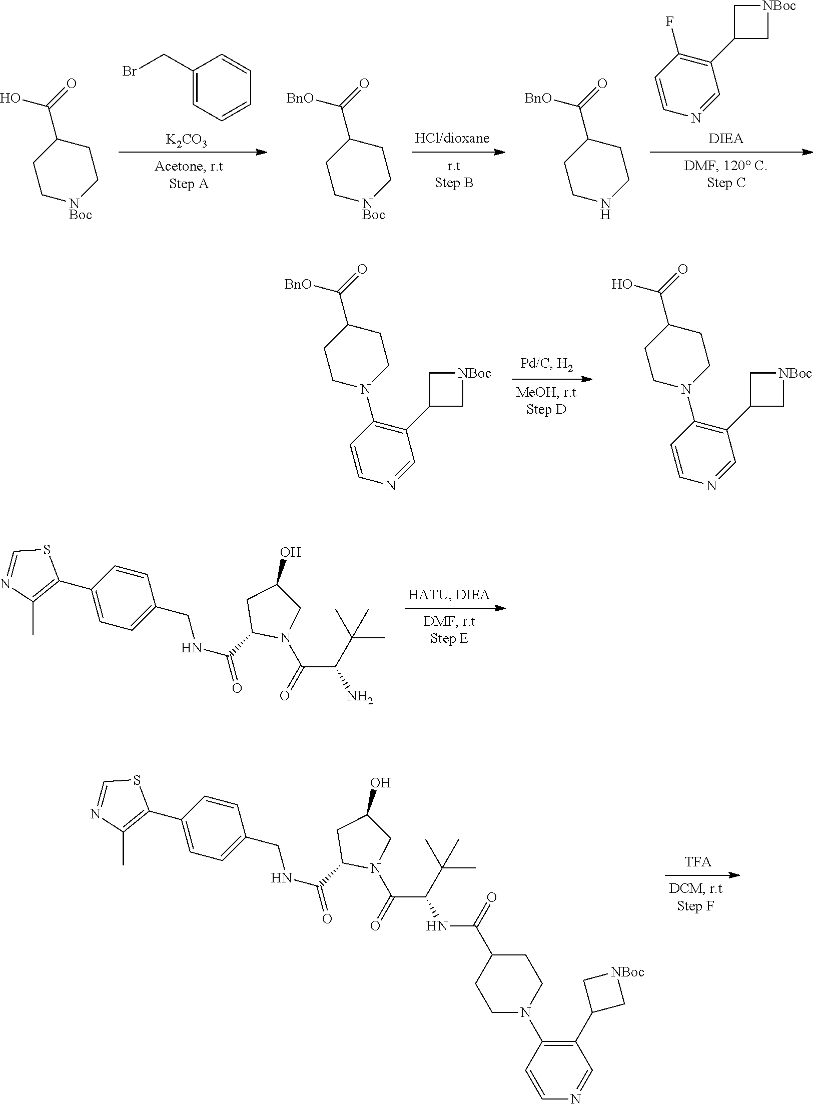

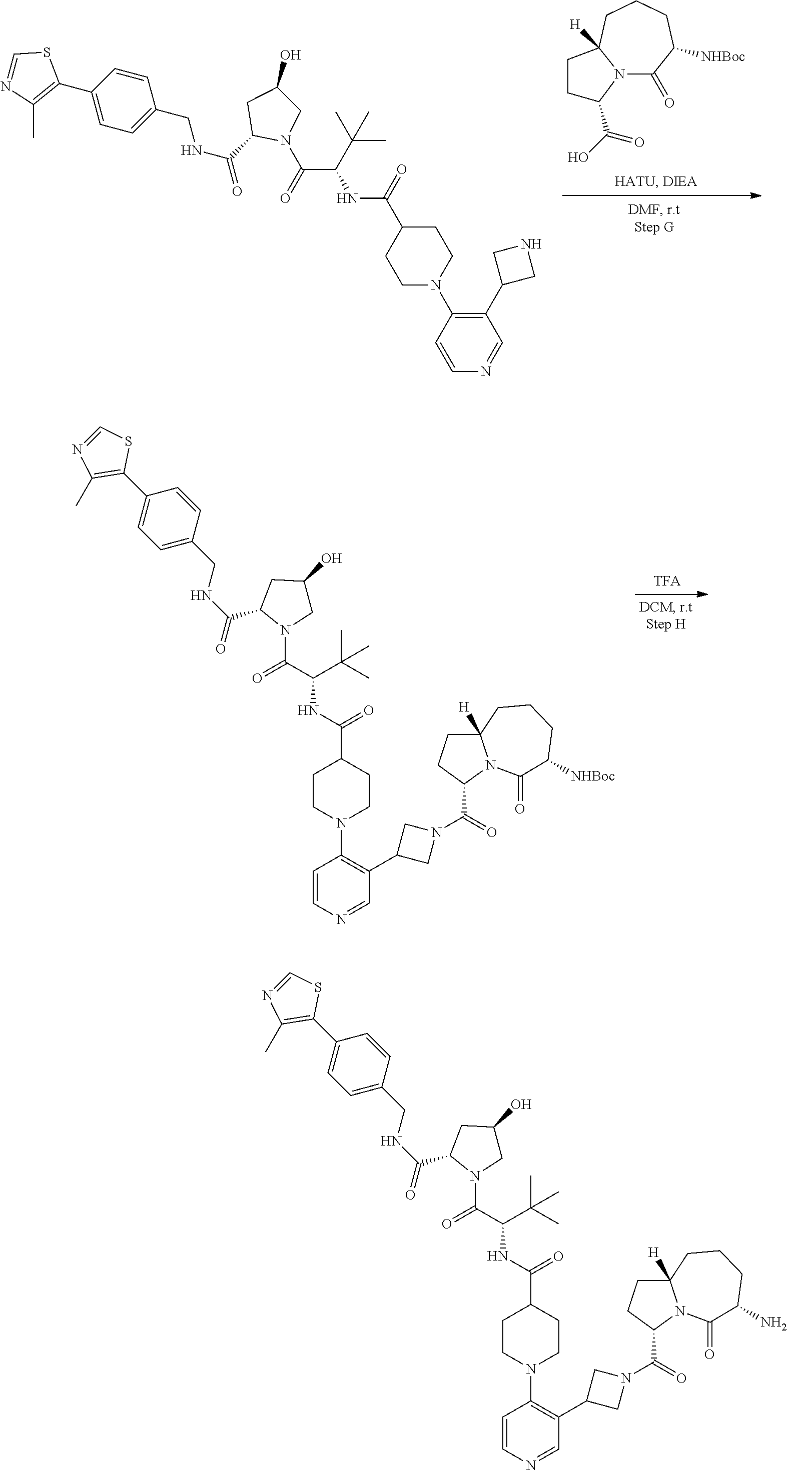 [SMILES: Cc1ncsc1-c1ccc(CNC(=O)[C@@H]2C[C@@H](O)CN2C(=O)[C@@H](NC(=O)C2CCN(c3ccncc3C3CNC3)CC2)C(C)(C)C)cc1 ; CC(C)(C)OC(=O)N[C@H]1CCC[C@H]2CC[C@@H](C(=O)O)N2C1=O ; Cc1ncsc1-c1ccc(CNC(=O)[C@@H]2C[C@@H](O)CN2C(=O)[C@@H](NC(=O)C2CCN(c3ccncc3C3CN(C(=O)[C@@H]4CC[C@@H]5CCC[C@H](NC(=O)OC(C)(C)C)C(=O)N54)C3)CC2)C(C)(C)C)cc1 ; Cc1ncsc1-c1ccc(CNC(=O)[C@@H]2C[C@@H](O)CN2C(=O)[C@@H](NC(=O)C2CCN(c3ccncc3C3CN(C(=O)[C@@H]4CC[C@@H]5CCC[C@H](N)C(=O)N54)C3)CC2)C(C)(C)C)cc1]

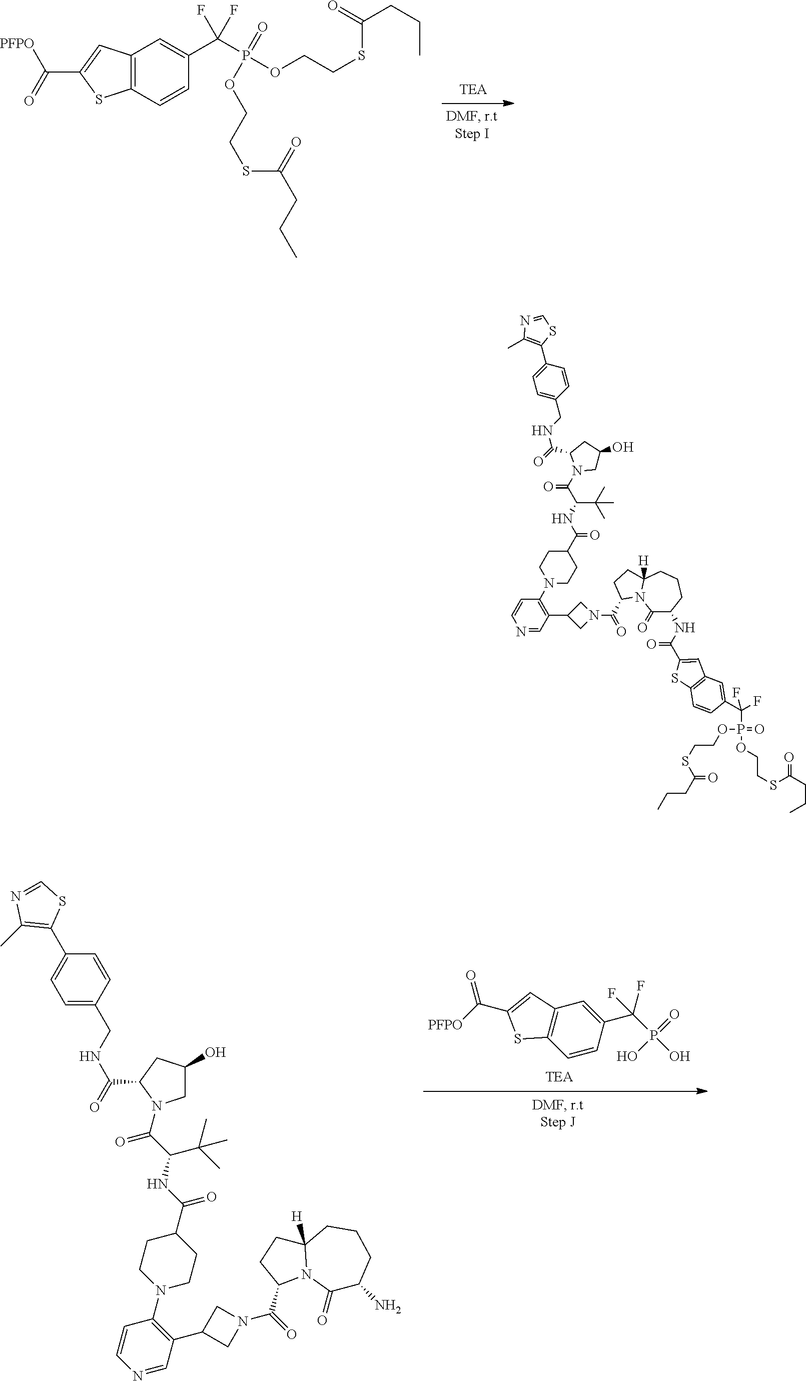

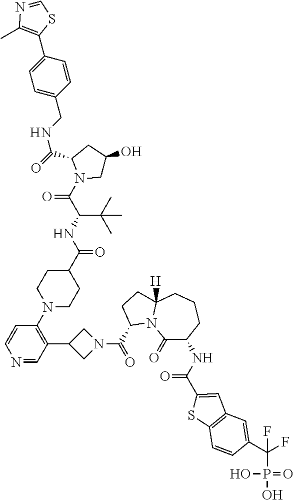 [SMILES: Cc1ncsc1-c1ccc(CNC(=O)[C@@H]2C[C@@H](O)CN2C(=O)[C@@H](NC(=O)C2CCN(c3ccncc3C3CN(C(=O)[C@@H]4CC[C@@H]5CCC[C@H](NC(=O)c6cc7cc(C(F)(F)P(=O)(O)O)ccc7s6)C(=O)N54)C3)CC2)C(C)(C)C)cc1]

### STEP A: 4-benzyl 1-(tert-butyl) piperidine-1,4-dicarboxylate

To a solution of 1-[(tert-butoxy)carbonyl]piperidine-4-carboxylic acid (5.0 g, 21.8 mmol, 1.0 eq.) in acetone (50 mL) were added (bromomethyl)benzene (4.1 g, 23.9 mmol, 1.1 eq.) and K₂CO₃ (6.0 g, 43.6 mmol, 2.0 eq.). The resulting mixture was stirred at room temperature for 2 hrs under N₂. After completion, the reaction mixture was concentrated under reduced pressure. The residue was purified by flash column chromatography on silica gel to afford 4-benzyl 1-tert-butyl piperidine-1,4-dicarboxylate (2.5 g, 7.82 mmol, 36%) as a white solid. LCMS (ESI): m/z=320 [M+H]⁺.

### STEP B: benzyl piperidine-4-carboxylate

To a solution of 4-benzyl 1-(tert-butyl) piperidine-1,4-dicarboxylate (2.5 g, 7.82 mmol, 1.0 eq.) in dioxane (10 mL) was added HCl/dioxane (5 mL, 2 M) and the resulting mixture was stirred at room temperature for 2 hrs under N₂. After completion, the reaction mixture was concentrated under reduced pressure to get crude benzyl piperidine-4-carboxylate (1.7 g, quant.) as a white solid, which was used in next step directly without further purification.

LCMS (ESI): m/z=220 [M+H]⁺.

### STEP C: benzyl 1-(3-(1-(tert-butoxycarbonyl)azetidin-3-yl)pyridin-4-yl)piperidine-4-carboxylate

To a solution of benzyl piperidine-4-carboxylate (250 mg, 1.14 mmol, 1.0 eq.) in DMF (3 mL) were added tert-butyl 3-(4-fluoropyridin-3-yl)azetidine-1-carboxylate (288 mg, 1.14 mmol, 1.0 eq.) and DIEA (443 mg, 3.42 mmol, 3.0 eq). The resulting mixture was stirred at 120° C. for 16 hrs under N₂. After completion, the reaction mixture was concentrated under reduced pressure. The residue was purified by Biotage® C18 column chromatography to get benzyl 1-(3-(1-(tert-butoxycarbonyl)azetidin-3-yl)pyridin-4-yl)piperidine-4-carboxylate (200 mg, 0.44 mmol, 39%) as a white solid. LCMS (ESI): m/z=452 [M+H]⁺.

### STEP D: 1-(3-(1-(tert-butoxycarbonyl)azetidin-3-yl)pyridin-4-yl)piperidine-4-carboxylic acid

To a solution of benzyl 1-(3-(1-(tert-butoxycarbonyl)azetidin-3-yl)pyridin-4-yl)piperidine-4-carboxylate (200 mg, 0.44 mmol, 1.0 eq.) in MeOH (4 mL) was added Pd/C (20 mg) under nitrogen. The suspension was degassed under vacuum and purged with H₂ several times. The resulting mixture was stirred at room temperature for 3 hrs. After completion, the suspension was filtered through a pad of Celite®, the filter cake was washed with MeOH (10 mL). The combined filtrates were concentrated to dryness to give 1-(3-(1-(tert-butoxycarbonyl)azetidin-3-yl)pyridin-4-yl)piperidine-4-carboxylic acid (130 mg, 0.36 mmol, 81%) as a white solid. LCMS (ESI): m/z=362 [M+H]⁺.

### STEP E: tert-butyl 3-(4-(4-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)carbamoyl)piperidin-1-yl)pyridin-3-yl)azetidine-1-carboxylate

To a solution of 1-(3-(1-(tert-butoxycarbonyl)azetidin-3-yl)pyridin-4-yl)piperidine-4-carboxylic acid (130 mg, 0.36 mmol, 1.0 eq.) and HATU (178 mg, 0.47 mmol, 1.3 eq.) in DMF (2 mL) were added (2S,4R)-1-((S)-2-amino-3,3-dimethylbutanoyl)-4-hydroxy-N-(4-(4-methylthiazol-5-yl)benzyl)pyrrolidine-2-carboxamide (155 mg, 0.36 mmol, 1.0 eq.) and DIEA (93 mg, 0.72 mmol, 2 eq.). The resulting mixture was stirred at room temperature for 2 hrs under N₂. After completion, the reaction mixture was purified by Biotage® C18 column chromatography to get tert-butyl 3-(4-(4-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)carbamoyl)piperidin-1-yl)pyridin-3-yl)azetidine-1-carboxylate (100 mg, 0.13 mmol, 36%) as a white solid. LCMS (ESI): m/z=774 [M+H]⁺.

### STEP F: 1-(3-(azetidin-3-yl)pyridin-4-yl)-N—((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)piperidine-4-carboxamide

To a solution of tert-butyl 3-(4-(4-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)carbamoyl)piperidin-1-yl)pyridin-3-yl)azetidine-1-carboxylate (100 mg, 0.13 mmol, 1.0 eq.) in DCM (3 mL) was added TFA (0.5 mL) and the reaction mixture was stirred at room temperature for 2 hrs under N₂. After completion, the reaction mixture was concentrated under reduced pressure to get crude 1-(3-(azetidin-3-yl)pyridin-4-yl)-N—((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)piperidine-4-carboxamide (87 mg, quant.) as a white solid, which was used in next step directly without further purification. LCMS (ESI): m/z=674 [M+H]⁺.

### STEP G: tert-butyl ((3S,6S,9aS)-3-(3-(4-(4-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)carbamoyl)piperidin-1-yl)pyridin-3-yl)azetidine-1-carbonyl)-5-oxooctahydro-1H-pyrrolo[1,2-a]azepin-6-yl)carbamate

To a solution of (3S,6S,9aS)-6-((tert-butoxycarbonyl)amino)-5-oxooctahydro-1H-pyrrolo[1,2-a]azepine-3-carboxylic acid (45 mg, 0.14 mmol, 1.1 eq.) and HATU (50 mg, 0.13 mmol, 1.3 eq.) in DMF(1 mL) were added 1-(3-(azetidin-3-yl)pyridin-4-yl)-N—((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)piperidine-4-carboxamide (87 mg, 0.13 mmol, 1 eq.) and DIEA (50 mg, 0.39 mmol, 3 eq.). The resulting mixture was stirred at room temperature for 2 hrs under N₂. After completion, the reaction mixture was purified by Biotage® C18 column chromatography to get tert-butyl ((3S,6S,9aS)-3-(3-(4-(4-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)carbamoyl)piperidin-1-yl)pyridin-3-yl)azetidine-1-carbonyl)-5-oxooctahydro-1H-pyrrolo[1,2-a]azepin-6-yl)carbamate (100 mg, 103 μmol, 80%) as a white solid. LCMS (ESI): m/z=968 [M+H]⁺.

### STEP H: 1-(3-(1-((3S,6S,9aS)-6-amino-5-oxooctahydro-1H-pyrrolo[1,2-a]azepine-3-carbonyl)azetidin-3-yl)pyridin-4-yl)-N—((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)piperidine-4-carboxamide

To a solution of tert-butyl ((3S,6S,9aS)-3-(3-(4-(4-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)carbamoyl)piperidin-1-yl)pyridin-3-yl)azetidine-1-carbonyl)-5-oxooctahydro-1H-pyrrolo[1,2-a]azepin-6-yl)carbamate (100 mg, 103 μmol, 1.0 eq.) in DCM (2 mL) was added TFA (0.5 mL) and the reaction mixture was stirred at room temperature for 2 hrs under N₂. After completion, the reaction mixture was concentrated under reduced pressure to get crude 1-(3-(1-((3S,6S,9aS)-6-amino-5-oxooctahydro-1H-pyrrolo[1,2-a]azepine-3-carbonyl)azetidin-3-yl)pyridin-4-yl)-N—((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)piperidine-4-carboxamide (89 mg, quant.) as a white solid, which was used in next step directly without further purification. LCMS (ESI): m/z=868 [M+H]⁺.

### STEP I: S,S((((difluoro(2-(((3S,6S,9aS)-3-(3-(4-(4-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)carbamoyl)piperidin-1-yl)pyridin-3-yl)azetidine-1-carbonyl)-5-oxooctahydro-1H-pyrrolo[,2-a]azepin-6-yl)carbamoyl)benzo[b]thiophen-5-yl)methyl)phosphoryl)bis(oxy))bis(ethane-2,1-diyl)) dibutanethioate

To a solution of 1-(3-(1-((3S,6S,9aS)-6-amino-5-oxooctahydro-1H-pyrrolo[1,2-a]azepine-3-carbonyl)azetidin-3-yl)pyridin-4-yl)-N—((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)piperidine-4-carboxamide (40 mg, 46 μmol, 1.0 eq.) in DMF (1 mL) were added perfluorophenyl 5-((bis(2-(butyrylthio)ethoxy)phosphoryl)difluoromethyl)benzo[b]thiophene-2-carboxylate (34 mg, 46 μmol, 1.0 eq.) and TEA (18 mg, 138 μmol, 3.0 eq.). The resulting mixture was stirred at room temperature for 2 hrs under N₂. After completion, the reaction mixture was concentrated under reduced pressure. The residue was purified by prep-HPLC to get S,S [symbol] ((((difluoro(2-(((3S,6S,9aS)-3-(3-(4-(4-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)carbamoyl)piperidin-1-yl)pyridin-3-yl)azetidine-1-carbonyl)-5-oxooctahydro-1H-pyrrolo[1,2-a]azepin-6-yl)carbamoyl)benzo[b]thiophen-5-yl)methyl)phosphoryl)bis(oxy))bis(ethane-2,1-diyl)) dibutanethioate (Compound 7, 20 mg, 14 μmol, 30%) as a white solid.

¹H NMR (400 MHz, DMSO-d6) δ 8.99 (s, 1H), 8.89-8.71 (m, 1H), 8.63-8.53 (m, 1H), 8.52-8.26 (m, 3H), 8.24-8.18 (m, 1H), 8.18-8.10 (m, 1H), 8.03-7.91 (m, 1H), 7.62-7.55 (m, 1H), 7.46-7.35 (m, 4H), 7.34-7.25 (m, 1H), 4.86-4.76 (m, 1H), 4.67-4.63 (m, 1H), 4.57-4.52 (m, 1H), 4.48-4.40 (m, 3H), 4.37-4.28 (m, 3H), 4.25-4.13 (m, 6H), 4.03-3.93 (m, 3H), 3.69-3.58 (m, 4H), 3.13 (t, J=6.0 Hz, 5H), 2.77-2.67 (m, 1H), 2.57-2.52 (m, 4H), 2.45 (s, 3H), 2.25-2.15 (m, 1H), 2.08-1.60 (m, 15H), 1.60-1.51 (m, 4H), 1.03-0.78 (m, 15H).

LCMS (ESI): m/z=1418.4 [M+H]⁺.

### STEP J: (difluoro(2-(((3S,6S,9aS)-3-(3-(4-(4-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)carbamoyl)piperidin-1-yl)pyridin-3-yl)azetidine-1-carbonyl)-5-oxooctahydro-1H-pyrrolo[1,2-a]azepin-6-yl)carbamoyl)benzo[b]thiophen-5-yl)methyl)phosphonic acid

To a solution of 1-(3-(1-((3S,6S,9aS)-6-amino-5-oxooctahydro-1H-pyrrolo[1,2-a]azepine-3-carbonyl)azetidin-3-yl)pyridin-4-yl)-N—((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)piperidine-4-carboxamide (40 mg, 46 μmol, 1.0 eq.) in DMF (1 mL) were added (difluoro(2-((perfluorophenoxy)carbonyl)benzo[b]thiophen-5-yl)methyl)phosphonic acid (22 mg, 46 μmol, 1.0 eq.) and TEA (18 mg, 138 μmol, 3.0 eq.). The resulting mixture was stirred at room temperature for 2 hrs under N₂. After completion, the reaction mixture was purified by prep-HPLC to get (difluoro(2-(((3S,6S,9aS)-3-(3-(4-(4-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)carbamoyl)piperidin-1-yl)pyridin-3-yl)azetidine-1-carbonyl)-5-oxooctahydro-1H-pyrrolo[1,2-a]azepin-6-yl)carbamoyl)benzo[b]thiophen-5-yl)methyl)phosphonic acid (Compound 8, 13 mg, 11 μmol, 24%) as a white solid.

¹H NMR (400 MHz, DMSO-d6) δ 8.99 (s, 1H), 8.82-8.67 (m, 1H), 8.62-8.53 (m, 1H), 8.52-8.24 (m, 3H), 8.17-8.05 (m, 2H), 8.02-7.94 (m, 1H), 7.64-7.55 (m, 1H), 7.48-7.37 (m, 4H), 7.32-7.25 (m, 1H), 4.87-4.75 (m, 1H), 4.68-4.64 (m, 1H), 4.57-4.51 (m, 1H), 4.45-3.95 (m, 10H), 3.75-3.57 (m, 4H), 3.21-3.04 (m, 2H), 2.79-2.66 (m, 1H), 2.45 (s, 3H), 2.21-1.64 (m, 16H), 1.01-0.85 (m, 9H).

LCMS (ESI): m/z=580.2 [M/2+H]⁺.

### Preparation of ((2-(((3S,6S,9aS)-3-(3-(4-(2-(4-(2,6-dioxopiperidin-3-yl)phenoxy)ethoxy)pyridin-3-yl)azetidine-1-carbonyl)-5-oxooctahydro-1H-pyrrolo[1,2-a]azepin-6-yl)carbamoyl)benzo[b]thiophen-5-yl)difluoromethyl)phosphonic acid (Compound 9) and S,S(((((2-(((3S,6S,9aS)-3-(3-(4-(2-(4-(2,6-dioxopiperidin-3-yl)phenoxy)ethoxy)pyridin-3-yl)azetidine-1-carbonyl)-5-oxooctahydro-1H-pyrrolo[1,2-a]azepin-6-yl)carbamoyl)benzo[b]thiophen-5-yl)difluoromethyl)phosphoryl)bis(oxy)) bis(ethane-2,1-diyl)) dibutanethioate (Compound 10)

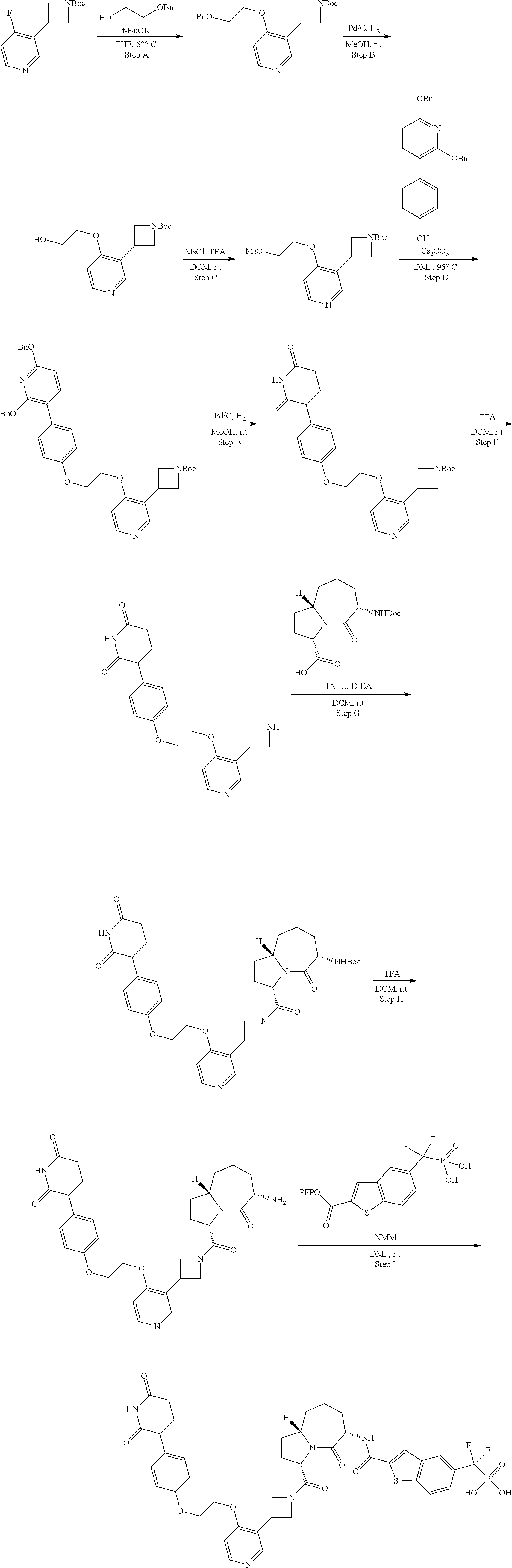

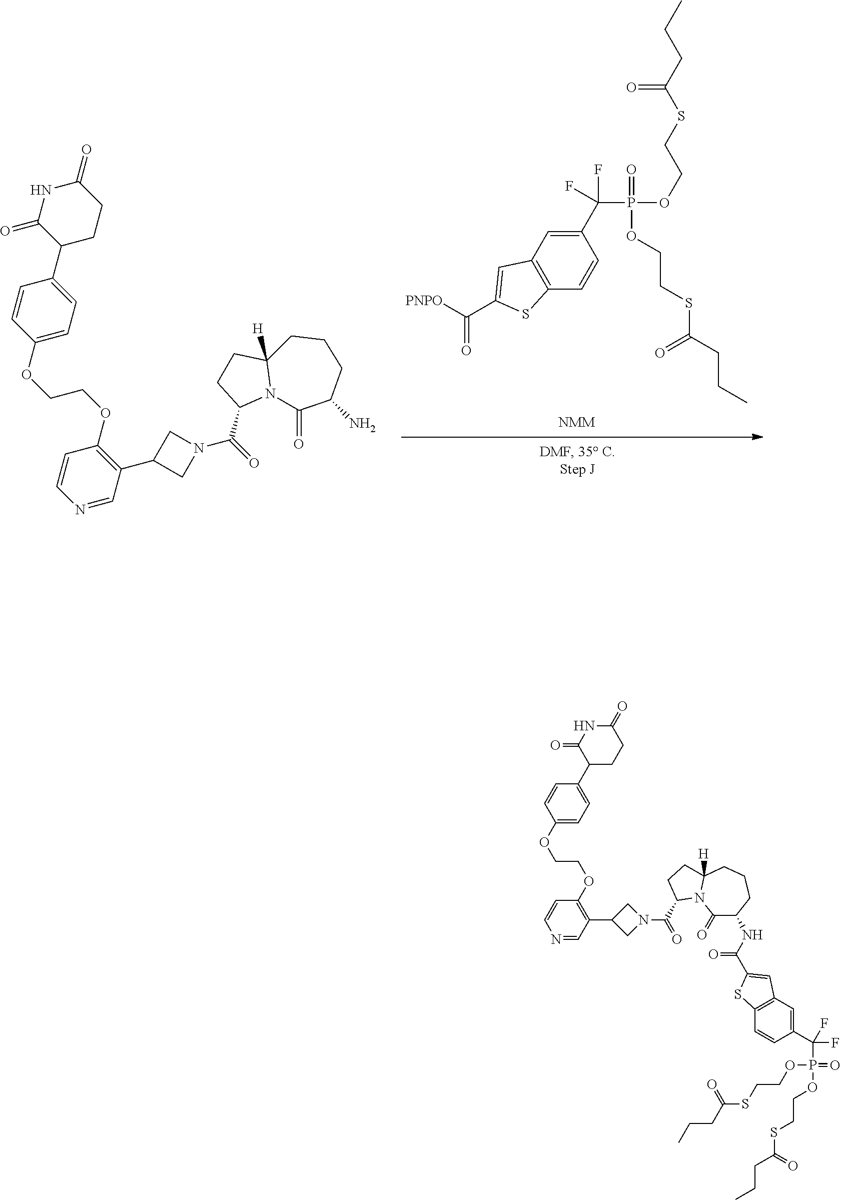

### STEP A: tert-butyl 3-(4-(2-(benzyloxy)ethoxy)pyridin-3-yl)azetidine-1-carboxylate

To a solution of tert-butyl 3-(4-fluoropyridin-3-yl)azetidine-1-carboxylate (500 mg, 1.98 mmol, 1.0 eq.) in THF (10 mL) was added t-BuOK (445 mg, 3.96 mmol, 2.0 eq.) at room temperature. The reaction mixture was stirred at 60° C. overnight. After completion, the reaction solvent was removed, the residue was purified by Biotage® C18 column chromatography to give tert-butyl 3-(4-(2-(benzyloxy)ethoxy)pyridin-3-yl)azetidine-1-carboxylate (556 mg, 1.45 mmol, 73%) as a white solid. LCMS (ESI): m/z=385 [M+H]⁺.

### STEP B: tert-butyl 3-(4-(2-hydroxyethoxy)pyridin-3-yl)azetidine-1-carboxylate

To a solution of tert-butyl 3-(4-(2-(benzyloxy)ethoxy)pyridin-3-yl)azetidine-1-carboxylate (556 mg, 1.45 mmol, 1.0 eq.) in MeOH (10 mL) was added Pd/C (55 mg). The suspension was degassed under vacuum and purged with H₂ several times. The reaction mixture was stirred at room temperature under H₂ for 4 hrs. After completion, the reaction mixture was filtered through a pad of Celite®, and washed with MeOH. The filtrate was concentrated under reduced pressure. The residue was purified by Biotage® C18 column chromatography to give tert-butyl 3-(4-(2-hydroxyethoxy)pyridin-3-yl)azetidine-1-carboxylate (350 mg, 1.19 mmol, 82%) as an oil. LCMS (ESI): m/z=295 [M+H]⁺.

### STEP C: tert-butyl 3-(4-(2-((methylsulfonyl)oxy)ethoxy)pyridin-3-yl)azetidine-1-carboxylate

To a solution of tert-butyl 3-(4-(2-hydroxyethoxy)pyridin-3-yl)azetidine-1-carboxylate (350 mg, 1.19 mmol, 1.0 eq.) in DCM (10 mL) were added TEA (301 mg, 2.98 mmol, 2.5 eq) and MsCl (205 mg, 1.79 mmol, 1.5 eq.) at room temperature. The reaction mixture was stirred at room temperature for 4 hrs. After completion, the reaction solvent was removed to give the crude tert-butyl 3-(4-(2-((methylsulfonyl)oxy)ethoxy)pyridin-3-yl)azetidine-1-carboxylate (442 mg, quant.) as an oil, which was used in next step directly without further purification. LCMS (ESI): m/z=373 [M+H]⁺.

### STEP D: tert-butyl 3-(4-(2-(4-(2,6-bis(benzyloxy)pyridin-3-yl)phenoxy)ethoxy)pyridin-3-yl)azetidine-1-carboxylate

To a solution of tert-butyl 3-(4-(2-((methylsulfonyl)oxy)ethoxy)pyridin-3-yl)azetidine-1-carboxylate (442 mg, 1.19 mmol, 1.0 eq.) in DMF (8 mL) was added 4-(2,6-bis(benzyloxy)pyridin-3-yl)phenol (502 mg, 1.31 mmol, 1.1 eq.) and Cs₂CO₃ (1.16 g, 3.57 mmol, 3 eq). The reaction mixture was stirred at 95° C. overnight. After completion, the reaction mixture was diluted with water, extracted with EtOAc (30 mL×2). The organic layers were combined and washed with brine (20 mL), dried over anhydrous Na₂SO₄, and concentrated under reduced pressure. The residue was purified by Biotage® C18 column chromatography to give tert-butyl 3-(4-(2-(4-(2,6-bis(benzyloxy)pyridin-3-yl)phenoxy)ethoxy)pyridin-3-yl)azetidine-1-carboxylate (380 mg, 0.58 mmol, 48%) as an oil. LCMS (ESI): m/z=660 [M+H]⁺.

### STEP E: tert-butyl 3-(4-(2-(4-(2,6-dioxopiperidin-3-yl)phenoxy)ethoxy)pyridin-3-yl)azetidine-1-carboxylate

To a solution of tert-butyl 3-(4-(2-(4-(2,6-bis(benzyloxy)pyridin-3-yl)phenoxy)ethoxy)pyridin-3-yl)azetidine-1-carboxylate (380 mg, 0.58 mmol, 1.0 eq.) in MeOH (5 mL) were added Pd/C (40 mg). The resulting mixture was stirred at room temperature under H₂ for 2 days. After completion, the suspension was filtered through a pad of Celite®, the filter cake was washed with MeOH (20 mL). The combined filtrates were concentrated to dryness. The residue was purified directly by Biotage® C18 column chromatography to give tert-butyl 3-(4-(2-(4-(2,6-dioxopiperidin-3-yl)phenoxy)ethoxy)pyridin-3-yl)azetidine-1-carboxylate (130 mg, 0.27 mmol, 47%) as a white solid. LCMS (ESI): m/z=482 [M+H]⁺.

### STEP F: 3-(4-(2-((3-(azetidin-3-yl)pyridin-4-yl)oxy)ethoxy)phenyl)piperidine-2,6-dione

To a solution of tert-butyl 3-(4-(2-(4-(2,6-dioxopiperidin-3-yl)phenoxy)ethoxy)pyridin-3-yl)azetidine-1-carboxylate (130 mg, 0.27 mmol, 1.0 eq.) in DCM (3 mL) was added TFA (1 mL). The reaction was stirred at room temperature for 2 hrs. After completion, the reaction solvent was removed to give the crude 3-(4-(2-((3-(azetidin-3-yl)pyridin-4-yl)oxy)ethoxy)phenyl)piperidine-2,6-dione (103 mg, quant.) as an oil, which was used in next step directly without further purification. LCMS (ESI): m/z=382 [M+H]⁺.

### STEP G: tert-butyl ((3S,6S,9aS)-3-(3-(4-(2-(4-(2,6-dioxopiperidin-3-yl)phenoxy)ethoxy)pyridin-3-yl)azetidine-1-carbonyl)-5-oxooctahydro-1H-pyrrolo[1,2-a]azepin-6-yl)carbamate

To a solution of 3-(4-(2-((3-(azetidin-3-yl)pyridin-4-yl)oxy)ethoxy) phenyl)piperidine-2,6-dione (103 mg, 0.27 mmol, 1.0 eq.), (3S,6S,9aS)-6-((tert-butoxycarbonyl)amino)-5-oxooctahydro-1H-pyrrolo[1,2-a]azepine-3-carboxylic acid (84 mg, 0.27 mmol, 1.0 eq.) and DIEA (89 mg, 0.68 mmol, 2.5 eq.) in DCM (10 mL) was added HATU (106 mg, 0.32 mmol, 1.2 eq.). The reaction mixture was stirred at room temperature overnight. After completion, the reaction mixture was diluted with H₂O (30 mL) and extracted with DCM (30 mL×2). The organic layers were combined and washed with brine (20 mL), dried over anhydrous Na₂SO₄, and concentrated under reduced pressure. The residue was purified by C18 column chromatography to give tert-butyl ((3S,6S,9aS)-3-(3-(4-(2-(4-(2,6-dioxopiperidin-3-yl)phenoxy)ethoxy)pyridin-3-yl)azetidine-1-carbonyl)-5-oxooctahydro-1H-pyrrolo[1,2-a]azepin-6-yl)carbamate (105 mg, 0.16 mmol, 58%) as a white solid. LCMS (ESI): m/z=676 [M+H]⁺.

### STEP H: 3-(4-(2-((3-(1-((3S,6S,9aS)-6-amino-5-oxooctahydro-1H-pyrrolo[1,2-a]azepine-3-carbonyl)azetidin-3-yl)pyridin-4-yl)oxy)ethoxy)phenyl)piperidine-2,6-dione

To a solution of tert-butyl ((3S,6S,9aS)-3-(3-(4-(2-(4-(2,6-dioxopiperidin-3-yl)phenoxy)ethoxy)pyridin-3-yl)azetidine-1-carbonyl)-5-oxooctahydro-1H-pyrrolo[1,2-a]azepin-6-yl)carbamate (40 mg, 59 μmol, 1.0 eq.) in DCM (1.5 mL) was added TFA (0.5 mL). The reaction was stirred at room temperature for 2 hrs. After completion, the reaction solvent was removed to give the crude 3-(4-(2-((3-(1-((3S,6S,9aS)-6-amino-5-oxooctahydro-1H-pyrrolo[1,2-a]azepine-3-carbonyl)azetidin-3-yl)pyridin-4-yl)oxy)ethoxy)phenyl) piperidine-2,6-dione (34 mg, quant.) as an oil, which was used in next step directly without further purification. LCMS (ESI): m/z=576 [M+H]+

### STEP I: 3-(4-(2-((3-(1-((3S,6S,9aS)-6-amino-5-oxooctahydro-1H-pyrrolo[1,2-a]azepine-3-carbonyl)azetidin-3-yl)pyridin-4-yl)oxy)ethoxy)phenyl)piperidine-2,6-dione

To a solution of 3-(4-(2-((3-(1-((3S,6S,9aS)-6-amino-5-oxooctahydro-1H-pyrrolo[1,2-a]azepine-3-carbonyl)azetidin-3-yl)pyridin-4-yl)oxy)ethoxy)phenyl)piperidine-2,6-dione (34 mg, 59 μmol, 1.0 eq.) in DMF (2 mL) were added NMM (0.2 mL) and (difluoro(2-((perfluorophenoxy)carbonyl)benzo[b]Thiophen-5-yl)methyl)phosphonic acid (28 mg, 59 μmol, 1.0 eq). The reaction mixture was stirred at room temperature for 2 hrs. After completion, the residue was purified by prep-HPLC to give ((2-(((3S,6S,9aS)-3-(3-(4-(2-(4-(2,6-dioxopiperidin-3-yl)phenoxy)ethoxy)pyridin-3-yl)azetidine-1-carbonyl)-5-oxooctahydro-1H-pyrrolo[1,2-a]azepin-6-yl)carbamoyl)benzo[b]thiophen-5-yl)difluoromethyl)phosphonic acid (Compound 9, 10.3 mg, 11.9 μmol, 20%) as a white solid.

¹H NMR (400 MHz, DMSO 6 10.79 (d, J=4.3 Hz, 1H), 8.80-8.58 (m, 3H), 8.30-8.18 (m, 1H), 8.14-7.99 (m, 2H), 7.67-7.50 (m, 2H), 7.13 (d, J=8.4 Hz, 2H), 6.93 (d, J=7.0 Hz, 2H), 4.71-4.63 (m, 3H), 4.49-4.34 (m, 4H), 4.24-3.87 (m, 5H), 3.81-3.74 (m, 1H), 2.75-2.40 (m, 2H), 2.22-2.06 (m, 2H), 2.01-1.86 (m, 3H), 1.85-1.59 (m, 7H).

LCMS (ESI): m/z=866 [M+H]⁺.

### STEP J: S,S(((((2-(((3S,6S,9aS)-3-(3-(4-(2-(4-(2,6-dioxopiperidin-3-yl)phenoxy)ethoxy)pyridin-3-yl)azetidine-1-carbonyl)-5-oxooctahydro-1H-pyrrolo[1,2-a]azepin-6-yl)carbamoyl)benzo[b]thiophen-5-yl)difluoromethyl)phosphoryl)bis(oxy))bis(ethane-2,1-diyl)) dibutanethioate

To a solution of 3-(4-(2-((3-(1-((3S,6S,9aS)-6-amino-5-oxooctahydro-1H-pyrrolo[1,2-a]azepine-3-carbonyl)azetidin-3-yl)pyridin-4-yl)oxy)ethoxy)phenyl)piperidine-2,6-dione (55 mg, 0.11 mmol, 1.0 eq.) in DMF (2 mL) were added NMM (0.2 mL) and 4-nitrophenyl 5-((bis(2-(butyrylthio)ethoxy)phosphoryl)difluoromethyl)benzo[b]thiophene-2-carboxylate (75 mg, 0.11 mmol, 1.0 eq). The reaction mixture was stirred at 35° C. overnight. After completion, the residue was purified by prep-HPLC to give S,S [symbol] (((((2-(((3S,6S,9aS)-3-(3-(4-(2-(4-(2,6-dioxopiperidin-3-yl)phenoxy)ethoxy)pyridin-3-yl)azetidine-1-carbonyl)-5-oxooctahydro-1H-pyrrolo[1,2-a]azepin-6-yl)carbamoyl)benzo[b]thiophen-5-yl)difluoromethyl)phosphoryl)bis(oxy))bis(ethane-2,1-diyl)) dibutanethioate (Compound 10, 11.9 mg, 10.6 μmol, 9.5%) as white solid.

¹H NMR (400 MHz, DMSO-d6) δ 10.78 (d, J=4.2 Hz, 1H), 8.83 (d, J=7.6 Hz, 1H), 8.41-8.27 (m, 3H), 8.23-8.12 (m, 2H), 7.62-7.53 (m, 1H), 7.17-7.06 (m, 3H), 6.95-6.89 (m, 2H), 4.70-4.30 (m, 8H), 4.21-3.92 (m, 8H), 3.81-3.72 (m, 1H), 3.17-3.07 (m, 4H), 2.70-2.60 (m, 1H), 2.57-2.52 (m, 4H), 2.48-2.41 (m, 1H), 2.22-2.03 (m, 2H), 2.02-1.92 (m, 2H), 1.89-1.60 (m, 8H), 1.60-1.50 (m, 4H), 0.88-0.82 (m, 6H).

LCMS (ESI): m/z=1126.3 [M+H]⁺.

### Preparation of ((2-(((3S,6S,9aS)-3-(3-(4-(2-(2-(4-(2,6-dioxopiperidin-3-yl)phenoxy)ethoxy)ethoxy)pyridin-3-yl)azetidine-1-carbonyl)-5-oxooctahydro-1H-pyrrolo[1,2-a]azepin-6-yl)carbamoyl)benzo[b]thiophen-5-yl)difluoromethyl)phosphonic acid (Compound 11) and S,S(((((2-(((3S,6S,9aS)-3-(3-(4-(2-(4-(2,6-dioxopiperidin-3-yl)phenoxy)ethoxy)pyridin-3-yl)azetidine-1-carbonyl)-5-oxooctahydro-1H-pyrrolo[1,2-a]azepin-6-yl)carbamoyl)benzo[b]thiophen-5-yl)difluoromethyl)phosphoryl)bis(oxy))bis (ethane-2,1-diyl)) dibutanethioate (Compound 12)

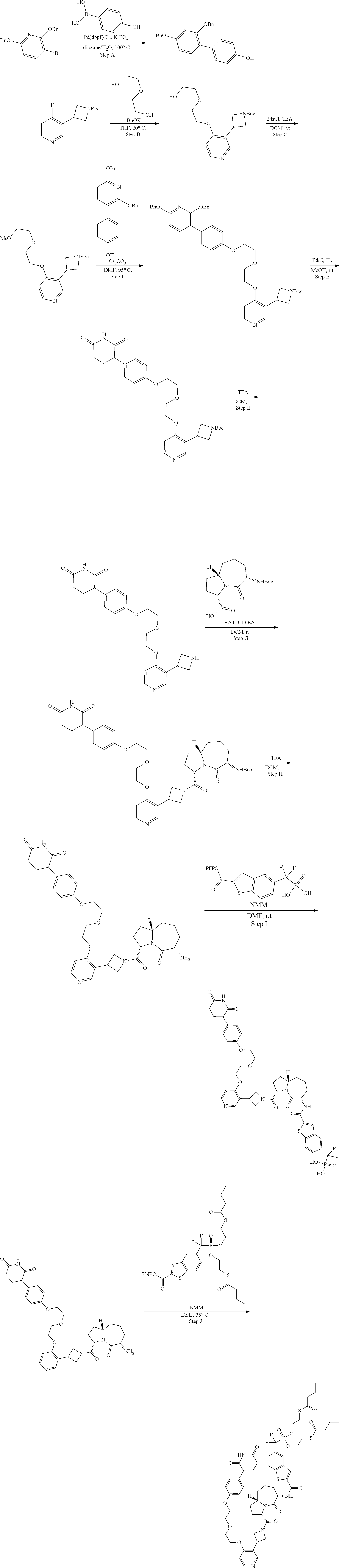

### STEP A: 4-(2,6-bis(benzyloxy)pyridin-3-yl)phenol

To a solution of 2,6-bis(benzyloxy)-3-bromopyridine (1.22 g, 3.3 mmol, 1.0 eq.) in dioxane (12 mL) were added (4-hydroxyphenyl)boronic acid (500 mg, 3.63 mmol, 1.1 eq.) and a solution of K₃PO₄ (1.75 g, 8.24 mmol, 2.5 eq.) in H₂O (4 mL) followed by Pd(dppf)Cl₂ (239 mg, 0.33 mmol, 0.1 eq.) at room temperature. The suspension was degassed under vacuum and purged with N₂ several times. The reaction mixture was stirred at 100° C. overnight. After completion, the suspension was filtered through a pad of Celite®, the filter cake was washed with EtOAc (20 mL). The combined filtrates were concentrated to dryness, the residue was purified by Biotage® C18 column chromatography to give 4-(2,6-bis(benzyloxy)pyridin-3-yl)phenol (1.1 g, 2.87 mmol, 87%) as a white solid. LCMS (ESI): m/z=384 [M+H]⁺.

### STEP B: tert-butyl 3-(4-(2-(2-hydroxyethoxy)ethoxy)pyridin-3-yl)azetidine-1-carboxylate

To a solution of tert-butyl 3-(4-fluoropyridin-3-yl)azetidine-1-carboxylate (500 mg, 1.98 mmol, 1.0 eq.) in THF (15 mL) was added 2,2 [symbol] oxybis(ethan-1-ol) (2.1 g, 19.8 mmol, 10 eq.) and t-BuOK (1.11 g, 9.91 mmol, 5.0 eq.) at room temperature. The reaction mixture was stirred at 60° C. overnight. After completion, the reaction solvent was removed, the residue was purified by Biotage® C18 column chromatography to give tert-butyl 3-(4-(2-(2-hydroxyethoxy)ethoxy)pyridin-3-yl)azetidine-1-carboxylate (500 mg, 1.48 mmol, 75%) as a white solid. LCMS (ESI): m/z=339 [M+H]⁺.

### STEP C: tert-butyl 3-(4-(2-(2-((methylsulfonyl)oxy)ethoxy)ethoxy)pyridin-3-yl)azetidine-1-carboxylate

To a solution of tert-butyl 3-(4-(2-(2-hydroxyethoxy)ethoxy)pyridin-3-yl)azetidine-1-carboxylate (500 mg, 1.48 mmol, 1.0 eq.) in DCM (10 mL) were added TEA (374 mg, 3.7 mmol, 2.5 eq.) and MsCl (254 mg, 2.22 mmol, 1.5 eq.) at room temperature. The reaction mixture was stirred at room temperature for 4 hrs. After completion, the reaction solvent was removed to give crude tert-butyl 3-(4-(2-(2-((methylsulfonyl)oxy)ethoxy)ethoxy)pyridin-3-yl)azetidine-1-carboxylate (616 mg, quant.) as an oil, which was used in next step directly without further purification. LCMS (ESI): m/z=417 [M+H]⁺.

### STEP D: tert-butyl 3-(4-(2-(2-(4-(2,6-bis(benzyloxy)pyridin-3-yl)phenoxy)ethoxy)ethoxy)pyridin-3-yl)azetidine-1-carboxylate

To a solution of tert-butyl 3-(4-(2-(2-((methylsulfonyl)oxy)ethoxy)ethoxy)pyridin-3-yl)azetidine-1-carboxylate (616 mg, 1.48 mmol, 1.0 eq.) in DMF (8 mL) were added 4-(2,6-bis(benzyloxy)pyridin-3-yl)phenol (624 mg, 1.63 mmol, 1.1 eq.) and Cs₂CO₃ (1.45 g, 4.44 mmol, 3 eq). The reaction mixture was stirred at 95° C. overnight. After completion, the reaction mixture was diluted with water (20 mL), extracted with EtOAc (30 mL×2). The organic layers were combined and washed with brine (20 mL), dried over anhydrous Na₂SO₄, and concentrated under reduced pressure. The residue was purified by Biotage® C18 column chromatography to give tert-butyl 3-(4-(2-(2-(4-(2,6-bis(benzyloxy)pyridin-3-yl)phenoxy)ethoxy)ethoxy) pyridin-3-yl)azetidine-1-carboxylate (703 mg, 1.0 mmol, 67%) as an oil. LCMS (ESI): m/z=704 [M+H]⁺.

### STEP E: tert-butyl 3-(4-(2-(2-(4-(2,6-dioxopiperidin-3-yl)phenoxy)ethoxy)ethoxy)pyridin-3-yl)azetidine-1-carboxylate

To a solution of tert-butyl 3-(4-(2-(4-(2,6-bis(benzyloxy)pyridin-3-yl)phenoxy)ethoxy)pyridin-3-yl)azetidine-1-carboxylate (703 mg, 1.0 mmol, 1.0 eq.) in MeOH (10 mL) was added Pd/C (100 mg). The suspension was degassed under vacuum and purged with H₂ several times. The resulting mixture was stirred at room temperature under H₂ for 2 days. After completion, the suspension was filtered through a pad of Celite®, the filter cake was washed with MeOH (20 mL). The combined filtrates were concentrated to dryness. The residue was purified by Biotage® C18 column chromatography to give tert-butyl 3-(4-(2-(2-(4-(2,6-dioxopiperidin-3-yl)phenoxy)ethoxy)ethoxy)pyridin-3-yl)azetidine-1-carboxylate (350 mg, 0.67 mmol, 67%) as a white solid. LCMS (ESI): m/z=526 [M+H]⁺.

### STEP F: 3-(4-(2-(2-((3-(azetidin-3-yl)pyridin-4-yl)oxy)ethoxy)ethoxy)phenyl)piperidine-2,6-dione

To a solution of tert-butyl 3-(4-(2-(2-(4-(2,6-dioxopiperidin-3-yl)phenoxy)ethoxy)ethoxy)pyridin-3-yl)azetidine-1-carboxylate (350 mg, 0.67 mmol, 1.0 eq.) in DCM (3 mL) was added TFA (1 mL). The reaction was stirred at room temperature for 2 hrs. After completion, the reaction solvent was removed to give crude 3-(4-(2-(2-((3-(azetidin-3-yl)pyridin-4-yl)oxy)ethoxy)ethoxy)phenyl)piperidine-2,6-dione (283 mg, quant.) as an oil, which was used in next step directly without further purification. LCMS (ESI): m/z=426 [M+H]⁺.

### STEP G: tert-butyl ((3S,6S,9aS)-3-(3-(4-(2-(2-(4-(2,6-dioxopiperidin-3-yl)phenoxy)ethoxy)ethoxy)pyridin-3-yl)azetidine-1-carbonyl)-5-oxooctahydro-1H-pyrrolo[1,2-a]azepin-6-yl)carbamate

To a solution of 3-(4-(2-(2-((3-(azetidin-3-yl)pyridin-4-yl)oxy)ethoxy)ethoxy)phenyl)piperidine-2,6-dione (283 mg, 0.67 mmol, 1.0 eq.), (3S,6S,9aS)-6-((tert-butoxycarbonyl)amino)-5-oxooctahydro-1H-pyrrolo[1,2-a]azepine-3-carboxylic acid (209 mg, 0.67 mmol, 1.0 eq.) and DIEA (216 mg, 1.68 mmol, 2.5 eq.) in DCM (10 mL) was added HATU (262 mg, 0.80 mmol, 1.2 eq.). The reaction mixture was stirred at room temperature overnight. After completion, the reaction mixture was diluted with H₂O (30 mL) and extracted with DCM (30 mL×2). The organic layers were combined and washed with brine (20 mL), dried over anhydrous Na₂SO₄, and concentrated under reduced pressure. The residue was purified by flash column chromatography on silica gel to give tert-butyl ((3S,6S,9aS)-3-(3-(4-(2-(2-(4-(2,6-dioxopiperidin-3-yl)phenoxy)ethoxy)ethoxy)pyridin-3-yl)azetidine-1-carbonyl)-5-oxooctahydro-1H-pyrrolo[1,2-a]azepin-6-yl)carbamate (320 mg, 0.44 mmol, 66%) as a white solid. LCMS (ESI): m/z=720 [M+H]⁺.

### STEP H: 3-(4-(2-(2-((3-(1-((3S,6S,9aS)-6-amino-5-oxooctahydro-1H-pyrrolo[1,2-a]azepine-3-carbonyl)azetidin-3-yl)pyridin-4-yl)oxy)ethoxy)ethoxy)phenyl)piperidine-2,6-dione

To a solution of tert-butyl ((3S,6S,9aS)-3-(3-(4-(2-(2-(4-(2,6-dioxopiperidin-3-yl)phenoxy)ethoxy)ethoxy) pyridin-3-yl)azetidine-1-carbonyl)-5-oxooctahydro-1H-pyrrolo[1,2-a]azepin-6-yl)carbamate (70 mg, 97 μmol, 1.0 eq.) in DCM (1.5 mL) was added TFA (0.5 mL). The reaction was stirred at room temperature for 2 hrs. After completion, the reaction solvent was removed to give crude 3-(4-(2-(2-((3-(1-((3S,6S,9aS)-6-amino-5-oxooctahydro-1H-pyrrolo[1,2-a]azepine-3-carbonyl)azetidin-3-yl)pyridin-4-yl)oxy)ethoxy)ethoxy)phenyl)piperidine-2,6-dione (60 mg, quant.) as an oil, which was used in next step directly without further purification. LCMS (ESI): m/z=620 [M+H]+

### STEP I: ((2-(((3S,6S,9aS)-3-(3-(4-(2-(2-(4-(2,6-dioxopiperidin-3-yl)phenoxy)ethoxy)ethoxy)pyridin-3-yl)azetidine-1-carbonyl)-5-oxooctahydro-1H-pyrrolo[1,2-a]azepin-6-yl)carbamoyl)benzo[b]thiophen-5-yl)difluoromethyl)phosphonic acid

To a solution of 3-(4-(2-(2-((3-(1-((3S,6S,9aS)-6-amino-5-oxooctahydro-1H-pyrrolo[1,2-a]azepine-3-carbonyl)azetidin-3-yl)pyridin-4-yl)oxy)ethoxy)ethoxy)phenyl)piperidine-2,6-dione (60 mg, 97 μmol, 1.0 eq.) in DMF (2 mL) were added NMM (0.30 mL) and (difluoro(2-((perfluorophenoxy) carbonyl)benzo[b]Thiophen-5-yl)methyl)phosphonic acid (46 mg, 97 μmol, 1.0 eq). The reaction mixture was stirred at room temperature for 2 hrs. After completion, the residue was purified by prep-HPLC to give ((2-(((3S,6S,9aS)-3-(3-(4-(2-(2-(4-(2,6-dioxopiperidin-3-yl)phenoxy)ethoxy)ethoxy)pyridin-3-yl)azetidine-1-carbonyl)-5-oxooctahydro-1H-pyrrolo[1,2-a]azepin-6-yl)carbamoyl)benzo[b]thiophen-5-yl)difluoromethyl)phosphonic acid (Compound 11, 14.3 mg, 16.5 μmol, 17%) as a white solid.

¹H NMR (400 MHz, DMSO-d6) δ 10.80 (d, J=3.9 Hz, 1H), 8.81-8.65 (m, 1H), 8.40-7.86 (m, 5H), 7.63-7.44 (m, 1H), 7.21 (d, J=6.1 Hz, 0.5H), 7.10 (t, J=9.3 Hz, 2H), 7.00 (d, J=5.8 Hz, 0.5H), 6.86 (t, J=9.2 Hz, 2H), 4.76-4.58 (m, 2H), 4.56-4.45 (m, 1H), 4.39-4.07 (m, 7H), 3.94-3.75 (m, 7H), 2.71-2.57 (m, 1H), 2.47-2.41 (m, 1H), 2.20-2.04 (m, 2H), 2.02-1.91 (m, 2H), 1.88-1.56 (m, 8H).

LCMS (ESI): m/z=910.4 [M+H]⁺.

### STEP J. S,S((((2-(((3S,6S,9aS)-3-(3-(4-(2-(2-(4-(2,6-dioxopiperidin-3-yl)phenoxy)ethoxy)ethoxy)pyridin-3-yl)azetidine-1-carbonyl)-5-oxooctahydro-1H-pyrrolo[1,2-a]azepin-6-yl)carbamoyl)benzo[b]thiophen-5-yl)difluoromethyl)phosphoryl)bis(oxy))bis(ethane-2,1-diyl)) dibutanethioate

To a solution of 3-(4-(2-(2-((3-(1-((3S,6S,9aS)-6-amino-5-oxooctahydro-1H-pyrrolo[1,2-a]azepine-3-carbonyl)azetidin-3-yl)pyridin-4-yl)oxy)ethoxy)ethoxy)phenyl)piperidine-2,6-dione (73 mg, 0.12 mmol, 1.0 eq.) in DMF (2 mL) were added NMM (0.2 mL) and 4-nitrophenyl 5-((bis(2-(butyrylthio)ethoxy)phosphoryl)difluoromethyl)benzo[b]thiophene-2-carboxylate (83 mg, 0.12 mmol, 1.0 eq). The reaction mixture was stirred at 35° C. overnight. After completion, the residue was purified by prep-HPLC to give S,S [symbol] (((((2-(((3S,6S,9aS)-3-(3-(4-(2-(4-(2,6-dioxopiperidin-3-yl)phenoxy)ethoxy)pyridin-3-yl)azetidine-1-carbonyl)-5-oxooctahydro-1H-pyrrolo[1,2-a]azepin-6-yl)carbamoyl)benzo[b]thiophen-5-yl)difluoromethyl)phosphoryl)bis(oxy))bis(ethane-2,1-diyl)) dibutanethioate (Compound 12, 15.6 mg, 13.3 μmol, 11%) as a white solid.

¹H NMR (400 MHz, DMSO-d6) δ 10.78 (d, J=3.2 Hz, 1H), 8.90-8.75 (m, 1H), 8.44-8.26 (m, 3H), 8.23-8.13 (m, 2H), 7.64-7.53 (m, 1H), 7.19-7.53 (m, 3H), 6.88-6.76 (m, 2H), 4.72-4.60 (m, 1H), 4.56-3.88 (m, 15H), 3.86-3.72 (m, 5H), 3.15-3.07 (m, 4H), 2.63-2.53 (m, 4H), 2.20-2.05 (m, 2H), 2.05-1.86 (m, 4H), 1.85-1.63 (m, 7H), 1.62-1.72 (m, 5H), 0.90-0.77 (m, 6H).

LCMS (ESI): m/z=1170 [M+H]⁺.

### Preparation of ((2-(((3S,6S,10aS)-3-((rel-3S,4R)-3-cyano-4-(3-((7-(2-(2,6-dioxopiperidin-3-yl)-1-oxoisoindolin-4-yl)hept-6-yn-1-yl)oxy)phenyl)pyrrolidine-1-carbonyl)-5-oxodecahydropyrrolo[1,2-a]azocin-6-yl)carbamoyl)benzo[b]thiophen-5-yl)difluoromethyl)phosphonic acid (Compound 13) and S,S(((((2-(((3S,6S,10aS)-3-((rel-3S,4R)-3-cyano-4-(3-((7-(2-(2,6-dioxopiperidin-3-yl)-1-oxoisoindolin-4-yl)hept-6-yn-1-yl)oxy)phenyl)pyrrolidine-1-carbonyl)-5-oxodecahydropyrrolo[1,2-a]azocin-6-yl)carbamoyl)benzo[b]thiophen-5-yl)difluoromethyl)phosphoryl)bis(oxy))bis(ethane-2,1-diyl)) dibutanethioate (Compound 14)

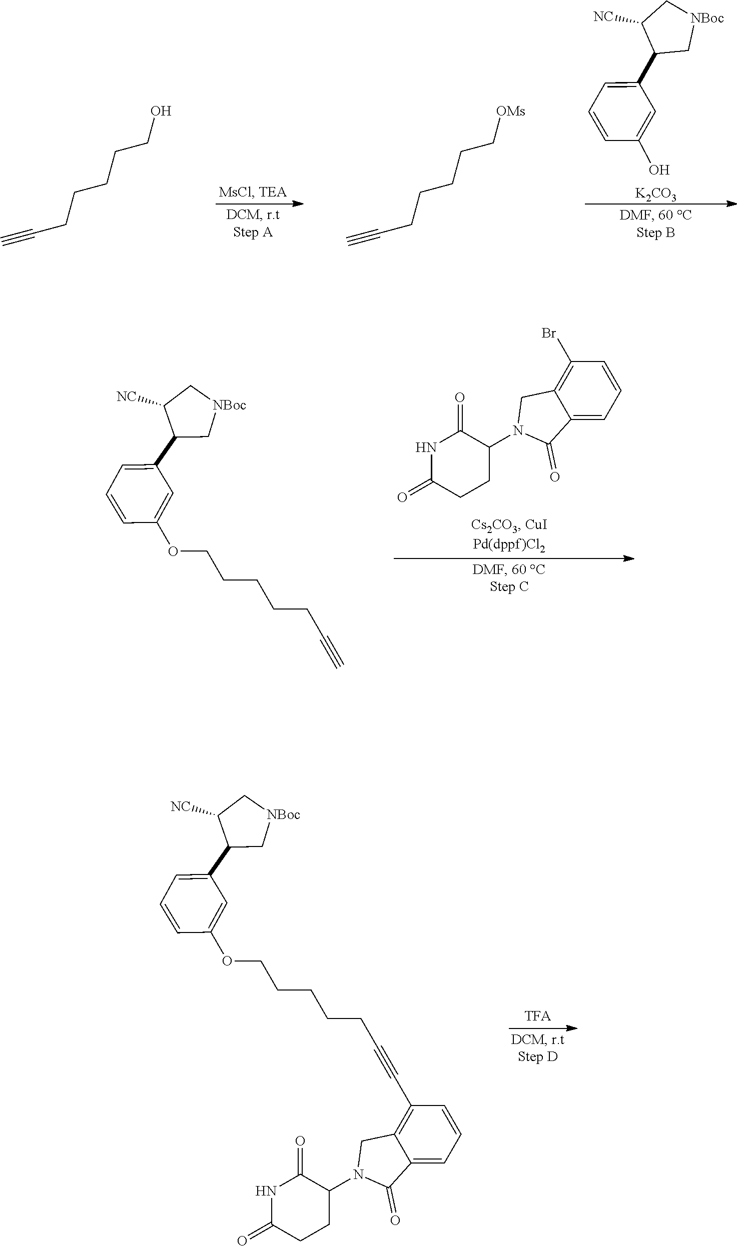

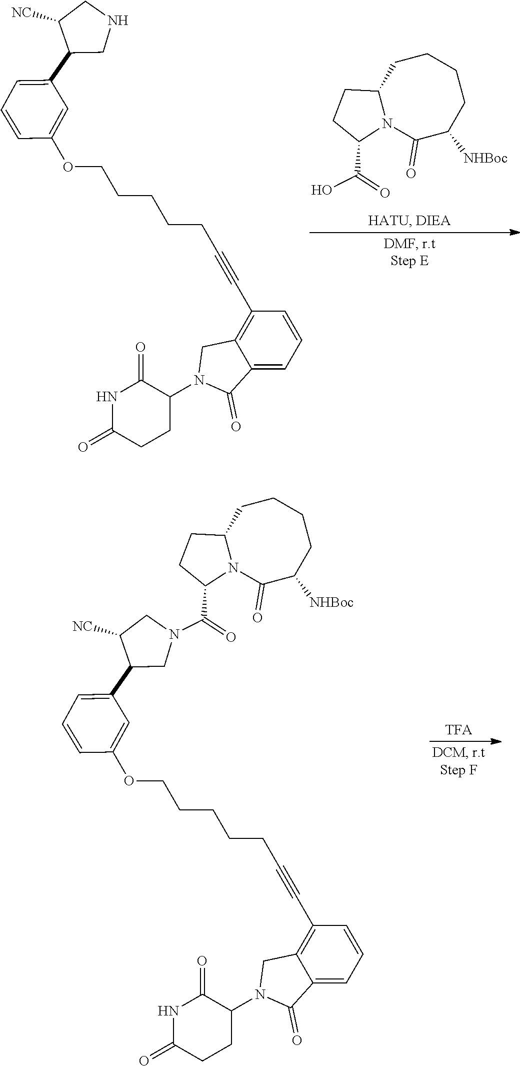 [SMILES: N#C[C@@H]1CNC[C@H]1c1cccc(OCCCCCC#Cc2cccc3c2CN(C2CCC(=O)NC2=O)C3=O)c1 ; CC(C)(C)OC(=O)N[C@H]1CCCC[C@H]2CC[C@@H](C(=O)O)N2C1=O ; CC(C)(C)OC(=O)N[C@H]1CCCC[C@H]2CC[C@@H](C(=O)N3C[C@@H](C#N)[C@H](c4cccc(OCCCCCC#Cc5cccc6c5CN(C5CCC(=O)NC5=O)C6=O)c4)C3)N2C1=O]

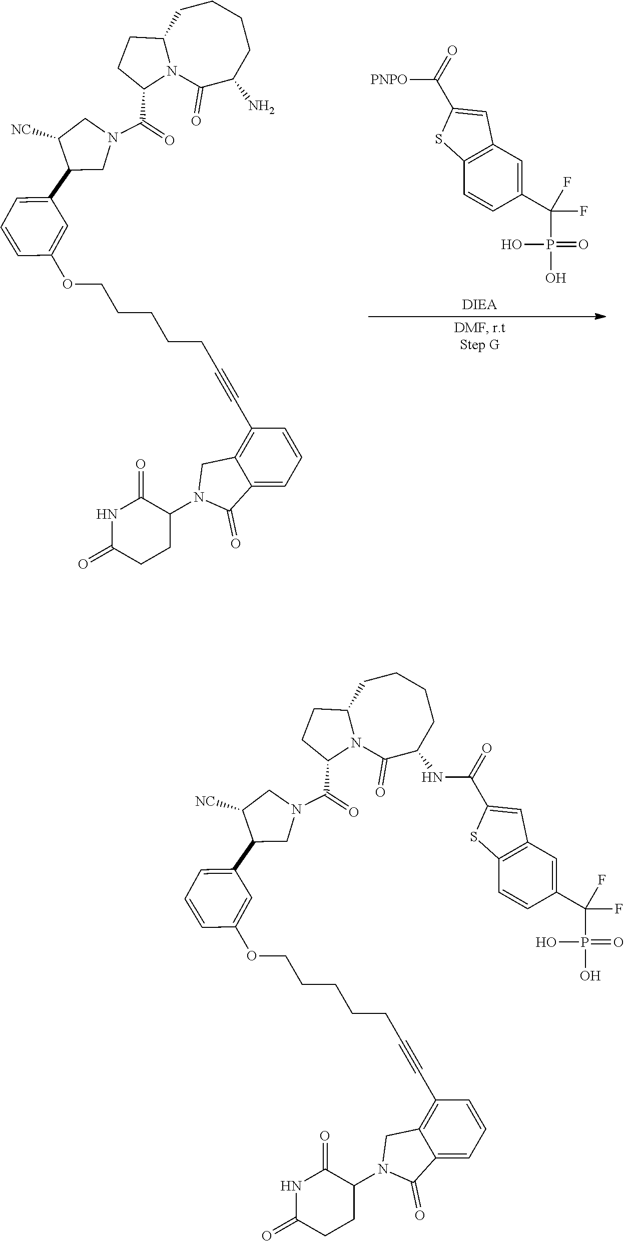

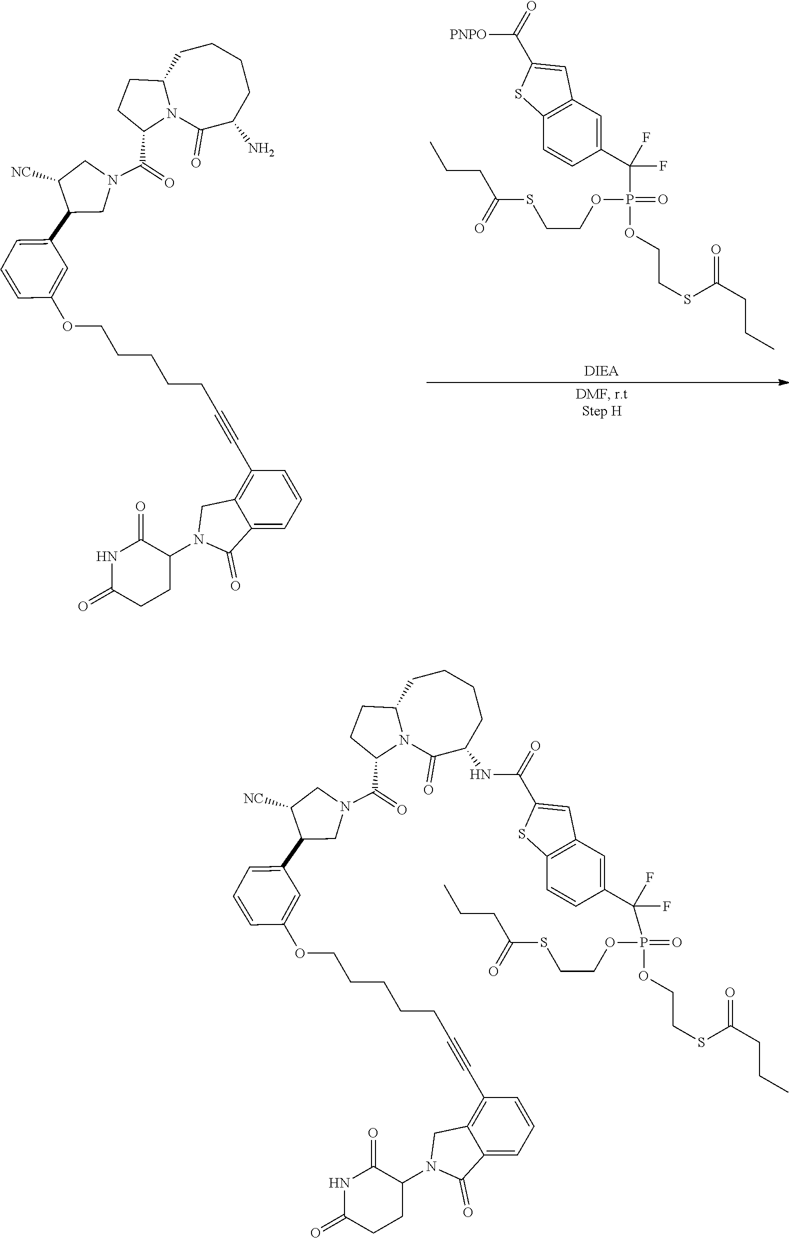

### STEP A: hept-6-yn-1-yl methanesulfonate

To a solution of hept-6-yn-1-ol (0_g, 44.6 mmol, 1.0 eq.) in DCM (60 mL) were added MsCl (10.0 g, 89.2 mmol, 2.0 eq.) and TEA (9.0, 89.2 mmol, 2.0 eq.). The resulting mixture was stirred at room temperature for 15 min under N₂. After completion, the reaction mixture was diluted with water (40 mL) and extracted with EtOAc (120 mL×3). The combined organic layer was washed with brine (100 mL×2), dried over anhydrous Na₂SO₄ and concentrated under reduced pressure to afford hept-6-yn-1-yl methanesulfonate (8 g, 42.1 mmol, 94%) as a colorless oil. ¹H NMR (400 MHz, DMSO-d6) δ 4.19 (t, J=6.4 Hz, 2H), 3.15 (s, 3H), 2.74 (t, J=2.5 Hz, 1H), 2.17 (td, J=6.7, 2.6 Hz, 2H), 1.73-1.62 (m, 2H), 1.51-1.40 (m, 4H).

### STEP B: rac-tert-butyl (3S,4R)-3-cyano-4-(3-(hept-6-yn-1-yloxy)phenyl)pyrrolidine-1-carboxylate

To a solution of hept-6-yn-1-yl methanesulfonate (800 mg, 4.2 mmol, 1.0 eq.) and rac-tert-butyl (3S,4R)-3-cyano-4-(3-hydroxyphenyl)pyrrolidine-1-carboxylate (1.21 g, 4.2 mmol, 1.0 eq.) in DMF (10 mL) was added K₂CO₃ (1.15 g, 8.4 mmol, 2.0 eq.). The mixture was stirred at 60° C. for 12 hrs. After completion, the mixture was filtered and the filtrate was purified by Biotage C18 column chromatography to afford rac-tert-butyl (3S,4R)-3-cyano-4-(3-(hept-6-yn-1-yloxy)phenyl)pyrrolidine-1-carboxylate (850 mg, 2.2 mmol, 50%) as a colorless oil. LCMS (ESI): m/z=383 [M+H]⁺.

### STEP C: rac-tert-butyl (3S,4R)-3-cyano-4-(3-((7-(2-(2,6-dioxopiperidin-3-yl)-1-oxoisoindolin-4-yl)hept-6-yn-1-yl)oxy)phenyl)pyrrolidine-1-carboxylate

To a solution of rac-tert-butyl (3S,4R)-3-cyano-4-(3-(hept-6-yn-1-yloxy)phenyl)pyrrolidine-1-carboxylate (630 mg, 1.6 mmol, 1.0 eq.) and 3-(4-bromo-1-oxoisoindolin-2-yl)piperidine-2,6-dione (520 mg, 1.6 mmol, 1.0 eq.) in DMF (10 mL) were added Cs₂CO₃ (1.6 g, 4.8 mmol, 3.0 eq.), CuI (63 mg, 0.32 mmol, 0.2 eq.) and Pd(dppf)Cl₂ (241 mg, 0.2 mmol, 0.2 eq.). The mixture was purged and degassed with N₂ 3 times and then stirred at 60° C. for 12 hrs. After completion, the mixture was filtered and the filtrate was purified by Biotage C18 column chromatography to afford rac-tert-butyl (3S,4R)-3-cyano-4-(3-((7-(2-(2,6-dioxopiperidin-3-yl)-1-oxoisoindolin-4-yl)hept-6-yn-1-yl)oxy)phenyl)pyrrolidine-1-carboxylate (340 mg, 0.54 mmol, 31%) as a gray solid. LCMS (ESI): m/z=625 [M+H]⁺.

### STEP D: rac-(3S,4R)-4-(3-((7-(2-(2,6-dioxopiperidin-3-yl)-1-oxoisoindolin-4-yl)hept-6-yn-1-yl)oxy)phenyl)pyrrolidine-3-carbonitrile

To a solution of rac-tert-butyl (3S,4R)-3-cyano-4-(3-((7-(2-(2,6-dioxopiperidin-3-yl)-1-oxoisoindolin-4-yl)hept-6-yn-1-yl)oxy)phenyl)pyrrolidine-1-carboxylate (170 mg, 0.27 mmol, 1.0 eq.) in DCM (1.8 mL) was added TFA (0.6 mL). The resulting mixture was stirred at room temperature for 2 hrs. After completion, the mixture was concentrated under reduced pressure to give rac-(3S,4R)-4-(3-((7-(2-(2,6-dioxopiperidin-3-yl)-1-oxoisoindolin-4-yl)hept-6-yn-1-yl)oxy)phenyl)pyrrolidine-3-carbonitrile (138 mg, quant.) as a yellow oil, which was used in next step directly without further purification. LCMS (ESI): m/z=525 [M+H]⁺.

### STEP E: tert-butyl ((3S,6S,10aS)-3-((rel-3S,4R)-3-cyano-4-(3-((7-(2-(2,6-dioxopiperidin-3-yl)-1-oxoisoindolin-4-yl)hept-6-yn-1-yl)oxy)phenyl)pyrroidine-1-carbonyl)-5-oxodecahydropyrrolo[1,2-a]azocin-6-yl)carbamate

To a solution of rac-(3S,4R)-4-(3-((7-(2-(2,6-dioxopiperidin-3-yl)-1-oxoisoindolin-4-yl)hept-6-yn-1-yl)oxy)phenyl)pyrrolidine-3-carbonitrile (138 mg, 0.26 mmol, 1.0 eq.) and (3S,6S,10aS)-6-((tert-butoxycarbonyl)amino)-5-oxodecahydropyrrolo[1,2-a]azocine-3-carboxylic acid (86 mg, 0.26 mmol, 1.0 eq.) in DMF (2 mL) were added HATU (120 mg, 0.31 mmol, 1.2 eq.) and DIEA (102 mg, 0.78 mmol, 3 eq.). The resulting mixture was stirred at room temperature for 1 hr. After completion, the reaction mixture was purified by Biotage® C18 column chromatography to get tert-butyl ((3S,6S,10aS)-3-((rel-3S,4R)-3-cyano-4-(3-((7-(2-(2,6-dioxopiperidin-3-yl)-1-oxoisoindolin-4-yl)hept-6-yn-1-yl)oxy)phenyl)pyrrolidine-1-carbonyl)-5-oxodecahydropyrrolo[1,2-a]azocin-6-yl)carbamate (120 mg, 0.14 mmol, 55%) as a white solid. LCMS (ESI): m/z=833 [M+H]⁺.

### STEP F: (rel-3S,4R)-1-((3S,6S,10aS)-6-amino-5-oxodecahydropyrrolo[1,2-a]azocine-3-carbonyl)-4-(3-((7-(2-(2,6-dioxopiperidin-3-yl)-1-oxoisoindolin-4-yl)hept-6-yn-1-yl)oxy)phenyl)pyrrolidine-3-carbonitrile

To a solution of (rel-3S,4R)-1-((3S,6S,10aS)-6-amino-5-oxodecahydropyrrolo[1,2-a]azocine-3-carbonyl)-4-(3-((7-(2-(2,6-dioxopiperidin-3-yl)-1-oxoisoindolin-4-yl)hept-6-yn-1-yl)oxy)phenyl)pyrrolidine-3-carbonitrile (120 mg, 0.14 mmol, 1.0 eq.) in DCM (1.8 mL) was added TFA (0.6 mL) and the reaction mixture was stirred at room temperature for 2 hrs under N₂. After completion, the reaction mixture was concentrated under reduced pressure to get (rel-3S,4R)-1-((3S,6S,10aS)-6-amino-5-oxodecahydropyrrolo[1,2-a]azocine-3-carbonyl)-4-(3-((7-(2-(2,6-dioxopiperidin-3-yl)-1-oxoisoindolin-4-yl)hept-6-yn-1-yl)oxy)phenyl)pyrrolidine-3-carbonitrile (101 mg, 0.14 mmol, 96%) as a yellow oil, which was used in next step directly without further purification.

LCMS (ESI): m/z=733 [M+H]⁺.

### STEP G: ((2-(((3S,6S,10aS)-3-((rel-3S,4R)-3-cyano-4-(3-((7-(2-(2,6-dioxopiperidin-3-yl)-1-oxoisoindolin-4-yl)hept-6-yn-1-yl)oxy)phenyl)pyrrolidine-1-carbonyl)-5-oxodecahydropyrrolo[1,2-a]azocin-6-yl)carbamoyl)benzo[b]thiophen-5-yl)difluoromethyl)phosphonic acid

To a solution of (rel-3S,4R)-1-((3S,6S,10aS)-6-amino-5-oxodecahydropyrrolo[1,2-a]azocine-3-carbonyl)-4-(3-((7-(2-(2,6-dioxopiperidin-3-yl)-1-oxoisoindolin-4-yl)hept-6-yn-1-yl)oxy)phenyl)pyrrolidine-3-carbonitrile (30 mg, 41 μmol, 1.0 eq.) and (difluoro(2-((perfluorophenoxy)carbonyl)benzo[b]thiophen-5-yl)methyl)phosphonic acid (19 mg, 41 μmol, 1.0 eq.) in DMF(1 mL) was added DIEA (16 mg, 0.12 mmol, 3 eq.). The resulting mixture was stirred at room temperature for 15 min. After completion, the reaction mixture was purified by prep-HPLC to give ((2-(((3S,6S,10aS)-3-((rel-3S,4R)-3-cyano-4-(3-((7-(2-(2,6-dioxopiperidin-3-yl)-1-oxoisoindolin-4-yl)hept-6-yn-1-yl)oxy)phenyl)pyrrolidine-1-carbonyl)-5-oxodecahydropyrrolo[1,2-a]azocin-6-yl)carbamoyl)benzo[b]thiophen-5-yl)difluoromethyl)phosphonic acid (Compound 14, 12 mg, 12 μmol, 29%) as a white solid.

¹H NMR (400 MHz, DMSO-d6) δ 11.05-10.92 (m, 1H), 8.91-8.77 (m, 1H), 8.37-8.27 (m, 1H), 8.17-8.03 (m, 2H), 7.74-7.44 (m, 4H), 7.31-7.23 (m, 1H), 7.06-6.92 (m, 2H), 6.91-6.84 (m, 1H), 5.18-5.08 (m, 1H), 4.99-4.91 (m, 1H), 4.63-4.11 (m, 6H), 4.08-3.86 (m, 4H), 3.82-3.73 (m, 1H), 3.67-3.60 (m, 1H), 3.36-3.18 (m, 1H), 2.95-2.84 (m, 1H), 2.48-1.15 (m, 22H).

LCMS (ESI): m/z=1023.4 [M+H]⁺.

### STEP H: S,S(((((2-(((3S,6S,10aS)-3-((rel-3S,4R)-3-cyano-4-(3-((7-(2-(2,6-dioxopiperidin-3-yl)-1-oxoisoindolin-4-yl)hept-6-yn-1-yl)oxy)phenyl)pyrrolidine-1-carbonyl)-5-oxodecahydropyrrolo[1,2-a]azocin-6-yl)carbamoyl)benzo[b]thiophen-5-yl)difluoromethyl)phosphoryl)bis(oxy))bis(ethane-2,1-diyl)) dibutanethioate

To a solution of (rel-3S,4R)-1-((3S,6S,10aS)-6-amino-5-oxodecahydropyrrolo[1,2-a]azocine-3-carbonyl)-4-(3-((7-(2-(2,6-dioxopiperidin-3-yl)-1-oxoisoindolin-4-yl)hept-6-yn-1-yl)oxy)phenyl)pyrrolidine-3-carbonitrile (30 mg, 41 μmol, 1.0 eq.) and 4-nitrophenyl 5-((bis(2-(butyrylthio)ethoxy)phosphoryl)difluoromethyl)benzo[b]thiophene-2-carboxylate (28 mg, 41 μmol, 1.0 eq.) in DMF(1 mL) was added DIEA (16 mg, 0.12 mmol, 3 eq.). The resulting mixture was stirred at room temperature for 15 min. After completion, the reaction mixture was purified by prep-HPLC to give S,S [symbol] (((((2-(((3S,6S,10aS)-3-((rel-3S,4R)-3-cyano-4-(3-((7-(2-(2,6-dioxopiperidin-3-yl)-1-oxoisoindolin-4-yl)hept-6-yn-1-yl)oxy)phenyl)pyrrolidine-1-carbonyl)-5-oxodecahydropyrrolo[1,2-a]azocin-6-yl)carbamoyl)benzo[b]thiophen-5-yl)difluoromethyl)phosphoryl)bis(oxy))bis(ethane-2,1-diyl)) dibutanethioate (Compound 14, 15 mg, 12 μmol, 29%) as a white solid.

¹H NMR (400 MHz, DMSO-d6) δ 10.99 (s, 1H), 9.00-8.79 (m, 1H), 8.39-8.30 (m, 1H), 8.24-8.09 (m, 2H), 7.74-7.45 (m, 4H), 7.30-7.22 (m, 1H), 7.05-6.81 (m, 3H), 5.20-5.06 (m, 1H), 5.03-4.87 (m, 1H), 4.65-4.38 (m, 2H), 4.36-3.45 (m, 13H), 3.33-3.30 (m, 6H), 3.21-3.05 (m, 4H), 2.97-2.80 (m, 1H), 2.43-1.39 (m, 24H), 1.34-1.14 (m, 2H), 0.94-0.79 (m, 6H).

LCMS (ESI): m/z=1283.4 [M+H]⁺.

### Preparation of S,S(((((2-(((3S,6S,10aS)-3-((rel-3S,4R)-3-cyano-4-(3-(2-(2-((2-(2,6-dioxopiperidin-3-yl)-1-oxoisoindolin-4-yl)oxy)ethoxy)ethoxy)phenyl)pyrrolidine-1-carbonyl)-5-oxodecahydropyrrolo[1,2-a]azocin-6-yl)carbamoyl)benzo[b]thiophen-5-yl)difluoromethyl)phosphoryl)bis(oxy))bis(ethane-2,1-diyl)) dibutanethioate (Compound 15) and ((2-(((3S,6S,10aS)-3-((rel-3S,4R)-3-cyano-4-(3-(2-(2-((2-(2,6-dioxopiperidin-3-yl)-1-oxoisoindolin-4-yl)oxy)ethoxy)ethoxy)phenyl)pyrrolidine-1-carbonyl)-5-oxodecahydropyrrolo[1,2-a]azocin-6-yl)carbamoyl)benzo[b]thiophen-5-yl)difluoromethyl)phosphonic acid (Compound 16)

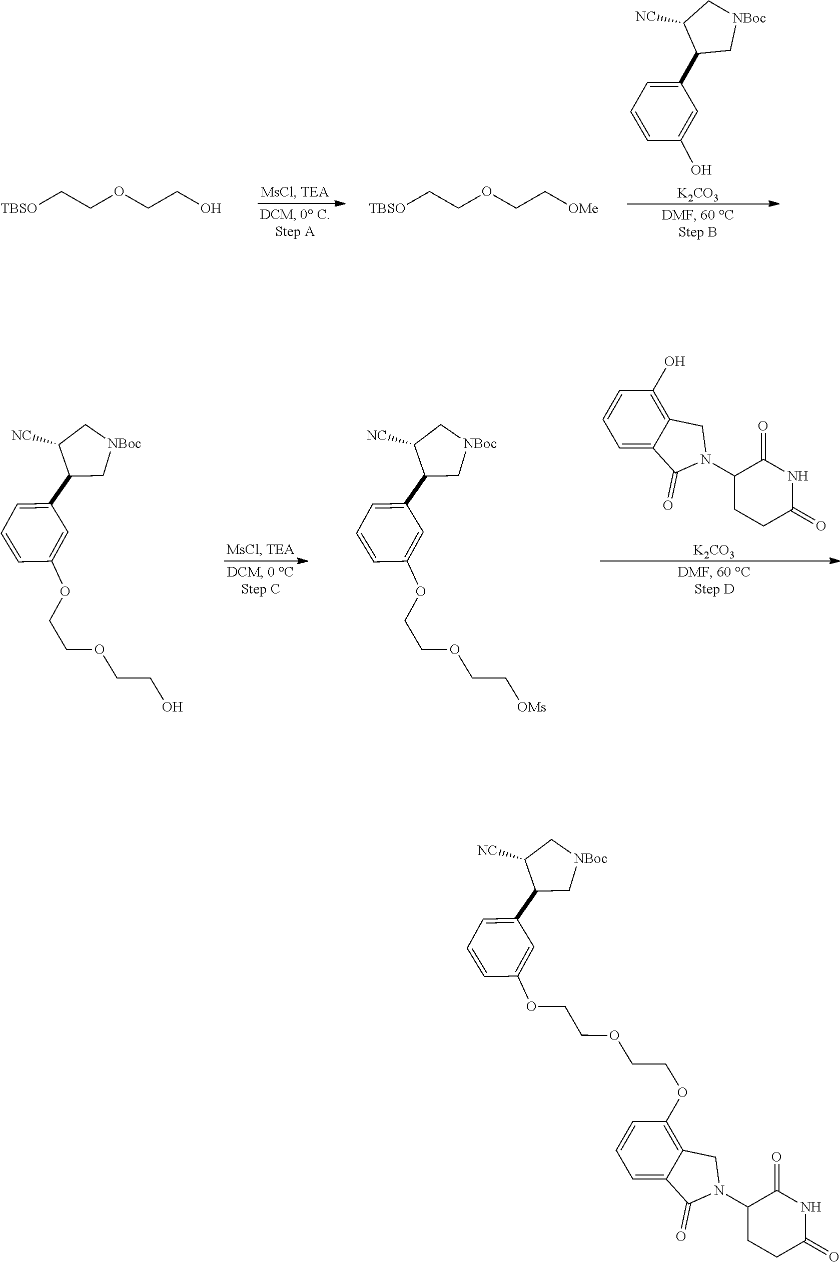

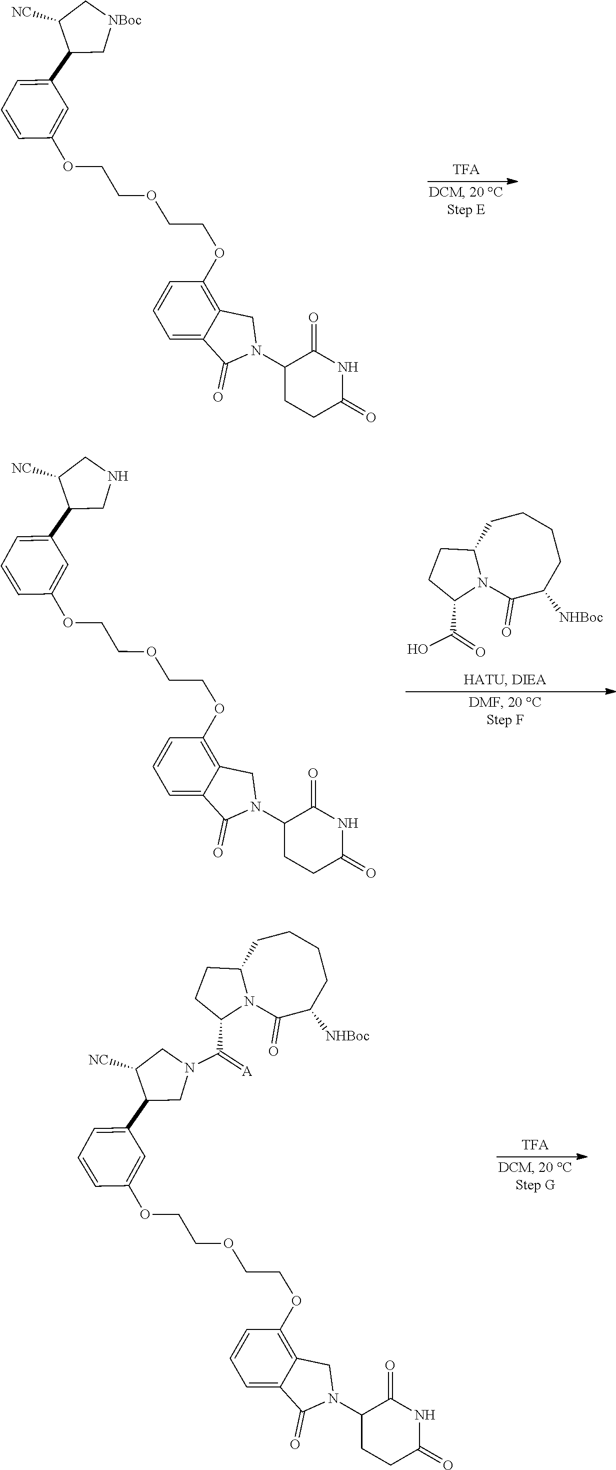

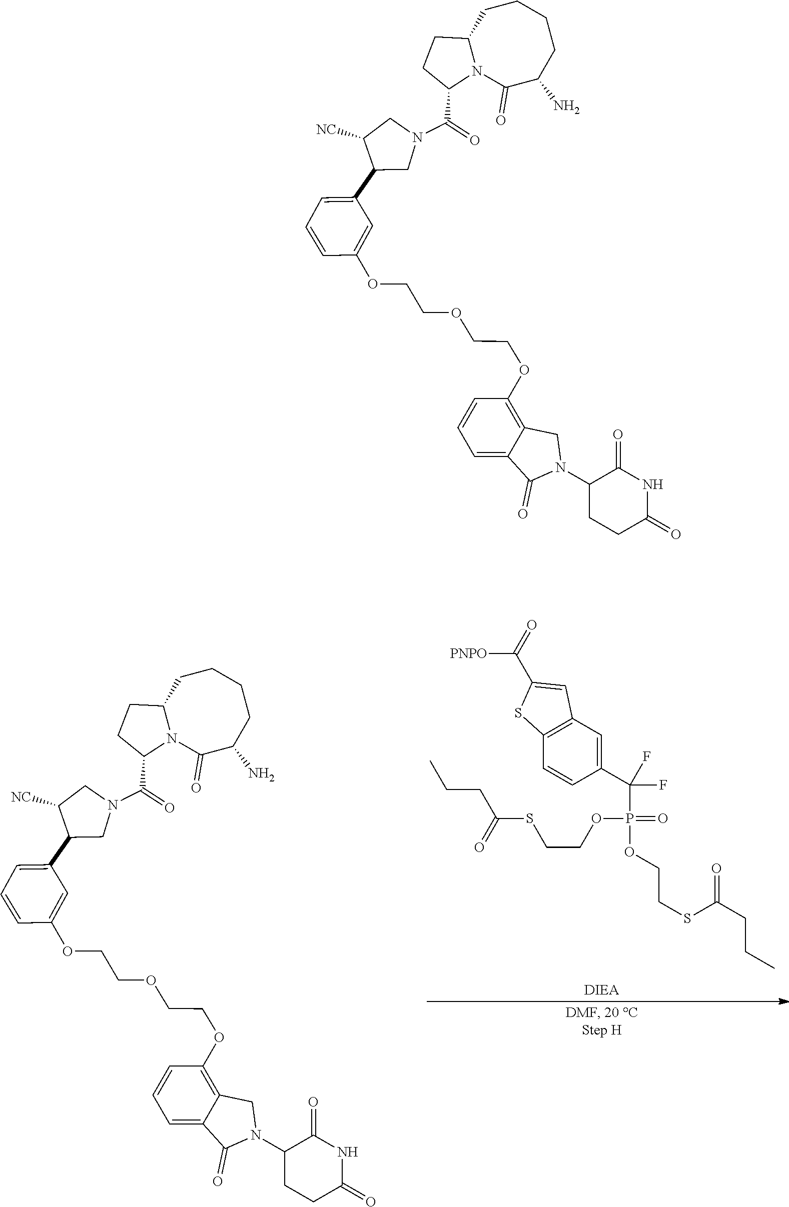

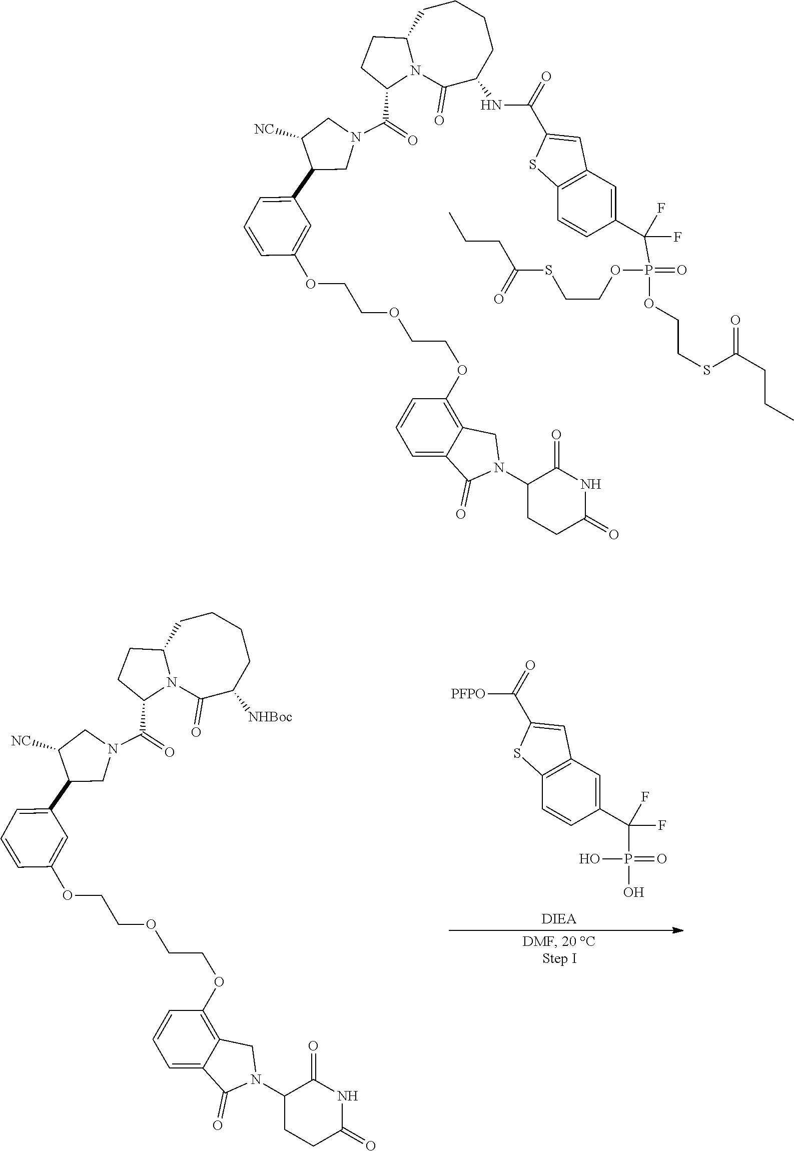

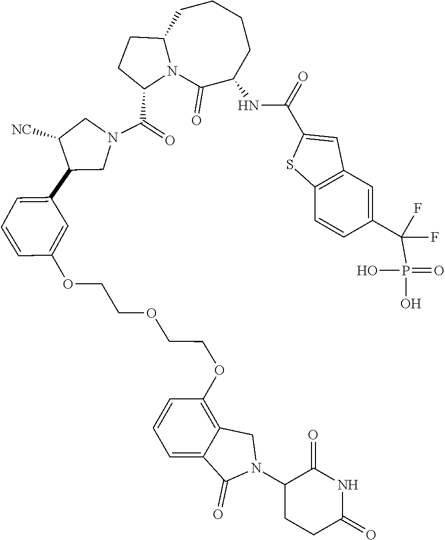 [SMILES: N#C[C@@H]1CN(C(=O)[C@@H]2CC[C@@H]3CCCC[C@H](NC(=O)c4cc5cc(C(F)(F)P(=O)(O)O)ccc5s4)C(=O)N32)C[C@H]1c1cccc(OCCOCCOc2cccc3c2CN(C2CCC(=O)NC2=O)C3=O)c1]

### STEP A: 2-(2-((tert-butyldimethylsilyl)oxy)ethoxy)ethyl methanesulfonate

To a solution of 2-(2-((tert-butyldimethylsilyl)oxy)ethoxy)ethan-1-ol (500 mg, 2.26 mmol, 1.0 eq.) and TEA (342 mg, 3.38 mmol, 1.5 eq.) in DCM (10 mL) was added MsCl (387 mg, 3.38 mmol, 1.5 eq.) dropwise. The reaction mixture was stirred at 0° C. for 1 hr. After completion, the reaction mixture was diluted with H₂O (30 mL) and extracted with DCM (30 mL×2). The combined organic layers were washed with brine (10 mL), dried over anhydrous Na₂SO₄, then concentrated under reduced pressure to give 2-(2-((tert-butyldimethylsilyl)oxy)ethoxy)ethyl methanesulfonate (500 mg, 1.67 mol, 74%) as a yellow liquid, which was used in next step directly without further purification. LCMS (ESI): m/z=299 [M+H]⁺.

### STEP B: rac-tert-butyl (3S,4R)-3-cyano-4-(3-(2-(2-hydroxyethoxy)ethoxy)phenyl)pyrrolidine-1-carboxylate

To a solution of 2-(2-((tert-butyldimethylsilyl)oxy)ethoxy)ethyl methanesulfonate (500 mg, 1.67 mmol, 1.0 eq.) and tert-butyl (3S,4R)-3-cyano-4-(3-hydroxyphenyl)pyrrolidine-1-carboxylate (3.5 g, 1.67 mmol, 1.0 eq.) in DMF (10 mL) was added K₂CO₃ (692 mg, 5.01 mmol, 3.0 eq.). The reaction mixture was stirred at 60° C. for 16 hrs. After completion, the reaction mixture was diluted with H₂O (30 mL) and extracted with EtOAc (30 mL×2). The combined organic layers were washed with brine (10 mL), dried over anhydrous Na₂SO₄, The residue was purified by flash column chromatography on silica gel to afford rac-tert-butyl (3S,4R)-3-cyano-4-(3-(2-(2-hydroxyethoxy)ethoxy)phenyl)pyrrolidine-1-carboxylate (260 mg, 690 umol, 41%) as a yellow oil. LCMS (ESI): m/z=399 [M+Na]⁺.

### STEP C: rac-tert-butyl (3S,4R)-3-cyano-4-(3-(2-(2-((methylsulfonyl)oxy)ethoxy)ethoxy)phenyl)pyrrolidine-1-carboxylate

To a solution of tert-butyl (3S,4R)-3-cyano-4-(3-(2-(2-hydroxyethoxy)ethoxy)phenyl)pyrrolidine-1-carboxylate (260 mg, 690 μmol, 1.0 eq.) and TEA (104 mg, 1.03 mmol, 1.5 eq.) in DCM (5 mL) was added MsCl (117 mg, 1.03 mmol, 1.5 eq.) dropwise. The reaction mixture was stirred at 0° C. for 1 hr. After completion, the reaction mixture was diluted with H₂O (10 mL) and extracted with DCM (10 mL×2). The combined organic layers were washed with brine (10 mL), dried over anhydrous Na₂SO₄, then concentrated under reduced pressure to give crude rac-tert-butyl (3S,4R)-3-cyano-4-(3-(2-(2-((methylsulfonyl)oxy)ethoxy)ethoxy)phenyl)pyrrolidine-1-carboxylate (300 mg, quant.) as a yellow liquid, which was used in next step directly without further purification. LCMS (ESI): m/z=399 [M+H-56]⁺.

### STEP D: rac-tert-butyl (3S,4R)-3-cyano-4-(3-(2-(2-((2-(2,6-dioxopiperidin-3-yl)-1-oxoisoindolin-4-yl)oxy)ethoxy)ethoxy)phenyl)pyrrolidine-1-carboxylate

To a solution of rac-tert-butyl (3S,4R)-3-cyano-4-(3-(2-(2-((methylsulfonyl)oxy)ethoxy)ethoxy)phenyl)pyrrolidine-1-carboxylate (300 mg, 660 μmol, 1.0 eq.) and 3-(4-hydroxy-1-oxoisoindolin-2-yl)piperidine-2,6-dione (6.17 g, 660 μmol, 1.0 eq.) in DMF (10 mL) was added K₂CO₃ (273 mg, 1.98 mmol, 3.0 eq.). The reaction mixture was stirred at 60° C. for 16 hrs. After completion, the reaction mixture was diluted with H₂O (30 mL) and extracted with EtOAc (30 mL×2). The combined organic layers were washed with brine (10 mL), dried over anhydrous Na₂SO₄ and concentrated under reduced pressure. The residue was purified by Biotage® C18 column chromatography to get rac-tert-butyl (3S,4R)-3-cyano-4-(3-(2-(2-((2-(2,6-dioxopiperidin-3-yl)-1-oxoisoindolin-4-yl)oxy)ethoxy)ethoxy)phenyl)pyrrolidine-1-carboxylate (180 mg, 0.29 mmol, 44%) as a white solid. LCMS (ESI): m/z=619 [M+H]⁺.

### STEP E: rac-(3S,4R)-4-(3-(2-(2-((2-(2,6-dioxopiperidin-3-yl)-1-oxoisoindolin-4-yl)oxy)ethoxy)ethoxy)phenyl)pyrrolidine-3-carbonitrile

To a solution of rac-tert-butyl (3S,4R)-3-cyano-4-(3-(2-(2-((2-(2,6-dioxopiperidin-3-yl)-1-oxoisoindolin-4-yl)oxy)ethoxy)ethoxy)phenyl)pyrrolidine-1-carboxylate (18_0 mg, 0.29 mmol, 1.0 eq.) in DCM (2 mL) was added TFA (0.6 mL) and the resulting mixture was stirred at room temperature for 2 hrs. After completion, the reaction mixture was concentrated under reduced pressure to get crude rac-(3S,4R)-4-(3-(2-(2-((2-(2,6-dioxopiperidin-3-yl)-1-oxoisoindolin-4-yl)oxy)ethoxy)ethoxy)phenyl)pyrrolidine-3-carbonitrile (150 mg, quant.) as a colorless oil, which was used in next step directly without further purification. LCMS (ESI): m/z=519 [M+H]⁺.

### STEP F: tert-butyl ((3S,6S,10aS)-3-((rel-3S,4R)-3-cyano-4-(3-(2-(2-((2-(2,6-dioxopiperidin-3-yl)-1-oxoisoindolin-4-yl)oxy)ethoxy)ethoxy)phenyl)pyrrolidine-1-carbonyl)-5-oxodecahydropyrrolo[1,2-a]azocin-6-yl)carbamate

To a mixture of (3S,6S,10aS)-6-((tert-butoxycarbonyl)amino)-5-oxodecahydropyrrolo[1,2-a]azocine-3-carboxylic acid (85 mg, 0.26 mmol, 0.9 eq.) and HATU (131 mg, 0.35 mmol, 1.2 eq.) in DMF (5 mL) were added DIEA (112 mg, 0.86 mmol, 3.0 eq.) and rac-(3S,4R)-4-(3-(2-(2-((2-(2,6-dioxopiperidin-3-yl)-1-oxoisoindolin-4-yl)oxy)ethoxy)ethoxy)phenyl)pyrrolidine-3-carbonitrile (150 mg, 0.29 mmol, 1.0 eq.). The resulting mixture was stirred at 25° C. for 2 hrs. After completion, the reaction mixture was purified directly by Biotage® C18 column chromatography to afford tert-butyl ((3S,6S,10aS)-3-((rel-3S,4R)-3-cyano-4-(3-(2-(2-((2-(2,6-dioxopiperidin-3-yl)-1-oxoisoindolin-4-yl)oxy)ethoxy)ethoxy)phenyl)pyrrolidine-1-carbonyl)-5-oxodecahydropyrrolo[1,2-a]azocin-6-yl)carbamate (120 mg, 0.15 mmol, 50%) as a white solid. LCMS (ESI): m/z=827 [M+H]⁺.

### STEP G: (rel-3S,4R)-1-((3S,6S,10aS)-6-amino-5-oxodecahydropyrrolo[1,2-a]azocine-3-carbonyl)-4-(3-(2-(2-((2-(2,6-dioxopiperidin-3-yl)-1-oxoisoindolin-4-yl)oxy)ethoxy)ethoxy)phenyl)pyrrolidine-3-carbonitrile

To a solution of tert-butyl ((3S,6S,10aS)-3-((rel-3S,4R)-3-cyano-4-(3-(2-(2-((2-(2,6-dioxopiperidin-3-yl)-1-oxoisoindolin-4-yl)oxy)ethoxy)ethoxy)phenyl)pyrrolidine-1-carbonyl)-5-oxodecahydropyrrolo[1,2-a]azocin-6-yl)carbamate (120 mg, 0.15 mmol, 1.0 eq.) in DCM (2 mL) was added TFA (0.6 mL) and the resulting mixture was stirred at room temperature for 2 hrs. After completion, the reaction mixture was concentrated under reduced pressure to get crude (rel-3S,4R)-1-((3S,6S,10aS)-6-amino-5-oxodecahydropyrrolo[1,2-a]azocine-3-carbonyl)-4-(3-(2-(2-((2-(2,6-dioxopiperidin-3-yl)-1-oxoisoindolin-4-yl)oxy)ethoxy)ethoxy)phenyl)pyrrolidine-3-carbonitrile (105 mg, quant.) as a colorless oil, which was used in next step directly without further purification. LCMS (ESI): m/z=727 [M+H]⁺.

### STEP H: S,S(((((2-(((3S,6S,10aS)-3-((rel-3S,4R)-3-cyano-4-(3-(2-(2-((2-(2,6-dioxopiperidin-3-yl)-1-oxoisoindolin-4-yl)oxy)ethoxy)ethoxy)phenyl)pyrrolidine-1-carbonyl)-5-oxodecahydropyrrolo[1,2-a]azocin-6-yl)carbamoyl)benzo[b]thiophen-5-yl)difluoromethyl)phosphoryl)bis(oxy))bis(ethane-2,1-diyl)) dibutanethioate

To a solution of (rel-3S,4R)-1-((3S,6S,10aS)-6-amino-5-oxodecahydropyrrolo[1,2-a]azocine-3-carbonyl)-4-(3-(2-(2-((2-(2,6-dioxopiperidin-3-yl)-1-oxoisoindolin-4-yl)oxy)ethoxy)ethoxy)phenyl)pyrrolidine-3-carbonitrile (50 mg, 69 μmol, 1.0 eq.) and 4-nitrophenyl 5-((bis(2-(butyrylthio)ethoxy)phosphoryl)difluoromethyl)benzo[b]thiophene-2-carboxylate (43 mg, 62 μmol, 0.9 eq.) in DMF (2 mL) was added DIEA (27 mg, 206 μmol, 3 eq.). The reaction mixture was stirred at room temperature for 2 hrs. After completion, the residue was purified by prep-HPLC to give S,S [symbol] (((((2-(((3S,6S,10aS)-3-((rel-3S,4R)-3-cyano-4-(3-(2-(2-((2-(2,6-dioxopiperidin-3-yl)-1-oxoisoindolin-4-yl)oxy)ethoxy)ethoxy)phenyl)pyrrolidine-1-carbonyl)-5-oxodecahydropyrrolo[1,2-a]azocin-6-yl)carbamoyl)benzo[b]thiophen-5-yl)difluoromethyl)phosphoryl)bis(oxy))bis(ethane-2,1-diyl)) dibutanethioate (Compound 15, 10.9 mg, 9 μmol, 12%) as a white solid.

¹H NMR (400 MHz, DMSO-d6) δ 10.97 (s, 1H), 8.95-8.85 (m, 1H), 8.37-8.32 (m, 1H), 8.23-8.12 (m, 2H), 7.61-7.55 (m, 1H), 7.49-7.41 (m, 1H), 7.34-7.20 (m, 3H), 7.06-6.85 (m, 3H), 5.15-5.03 (m, 1H), 5.01-4.88 (m, 1H), 4.62-4.44 (m, 1H), 4.30-4.12 (m, 10H), 4.02-3.92 (m, 1H), 3.87-3.74 (m, 5H), 3.33-3.20 (m, 1H), 3.16-3.10 (m, 4H), 2.97-2.84 (m, 1H), 2.57-2.54 (m, 3H), 2.38-1.45 (m, 21H), 1.30-1.17 (m, 3H), 0.88-0.84 (m, 6H).

LCMS (ESI): m/z=1277.4 [M+H]⁺.

### STEP I: ((2-(((3S,6S,10aS)-3-((rei-3S,4R)-3-cyano-4-(3-(2-(2-((2-(2,6-dioxopiperidin-3-yl)-1-oxoisoindolin-4-yl)oxy)ethoxy)ethoxy)phenyl)pyrrolidine-1-carbonyl)-5-oxodecahydropyrrolo[1,2-a]azocin-6-yl)carbamoyl)benzo[b]thiophen-5-yl)difluoromethyl)phosphonic acid

To a solution of (rel-3S,4R)-1-((3S,6S,10aS)-6-amino-5-oxodecahydropyrrolo[1,2-a]azocine-3-carbonyl)-4-(3-(2-(2-((2-(2,6-dioxopiperidin-3-yl)-1-oxoisoindolin-4-yl)oxy)ethoxy)ethoxy)phenyl)pyrrolidine-3-carbonitrile (50 mg, 69 μmol, 1.0 eq.) and (difluoro(2-((perfluorophenoxy)carbonyl)benzo[b]thiophen-5-yl)methyl)phosphonic acid (29 mg, 62 μmol, 0.9 eq.) in DMF (2 mL) was added DIEA (27 mg, 206 μmol, 3 eq.). The reaction mixture was stirred at room temperature for 2 hrs. After completion, the residue was purified by prep-HPLC to give ((2-(((3S,6S,10aS)-3-((rel-3S,4R)-3-cyano-4-(3-(2-(2-((2-(2,6-dioxopiperidin-3-yl)-1-oxoisoindolin-4-yl)oxy)ethoxy)ethoxy)phenyl)pyrrolidine-1-carbonyl)-5-oxodecahydropyrrolo[1,2-a]azocin-6-yl)carbamoyl)benzo[b]thiophen-5-yl)difluoromethyl)phosphonic acid (Compound 16, 10.5 mg, 10 μmol, 15%) as a white solid.

¹H NMR (400 MHz, DMSO-d6) δ 10.97 (s, 1H), 8.89-8.78 (m, 1H), 8.35-8.27 (m, 1H), 8.18-8.10 (m, 1H), 8.09-8.03 (m, 1H), 7.62-7.55 (m, 1H), 7.50-7.20 (m, 4H), 7.05-6.95 (m, 2H), 6.91-6.84 (m, 1H), 5.21-5.06 (m, 1H), 5.00-4.89 (m, 1H), 4.61-4.48 (m, 1H), 4.39-4.25 (m, 4H), 4.16-4.09 (m, 2H), 3.86-3.72 (m, 6H), 3.53-3.46 (m, 2H), 3.33-3.22 (m, 1H), 2.94-2.85 (m, 1H), 2.78-2.56 (m, 1H), 2.43-2.17 (m, 3H), 2.01-1.71 (m, 9H), 1.66-1.48 (m, 3H), 1.28-1.09 (m, 1H).

LCMS (ESI): m/z=1017.3 [M+H]⁺.

### Preparation of ((2-(((3S,6S,10aS)-3-((6R)-6-(1-(5-(4-(2,6-dioxopiperidin-3-yl)phenoxy)pentyl)-2-oxo-1,2-dihydropyridin-4-yl)-4-azaspiro[2.4]heptane-4-carbonyl)-5-oxodecahydropyrrolo[1,2-a]azocin-6-yl)carbamoyl)benzo[b]thiophen-5-yl)difluoromethyl)phosphonic acid (Compound 17) and S,S(((((2-(((3S,6S,10aS)-3-((6R)-6-(1-(5-(4-(2,6-dioxopiperidin-3-yl)phenoxy)pentyl)-2-oxo-1,2-dihydropyridin-4-yl)-4-azaspiro[2.4]heptane-4-carbonyl)-5-oxodecahydropyrrolo[1,2-a]azocin-6-yl)carbamoyl)benzo[b]thiophen-5-yl)difluoromethyl)phosphoryl)bis(oxy))bis(ethane-2,1-diyl)) dibutanethioate (Compound 18) and ((2-(((3S,6S,10aS)-3-((6R)-6-(2-((5-(4-(2,6-dioxopiperidin-3-yl)phenoxy)pentyl)oxy)pyridin-4-yl)-4-azaspiro[2.4]heptane-4-carbonyl)-5-oxodecahydropyrrolo[1,2-a]azocin-6-yl)carbamoyl)benzo[b]thiophen-5-yl)difluoromethyl)phosphonic acid (Compound 19)

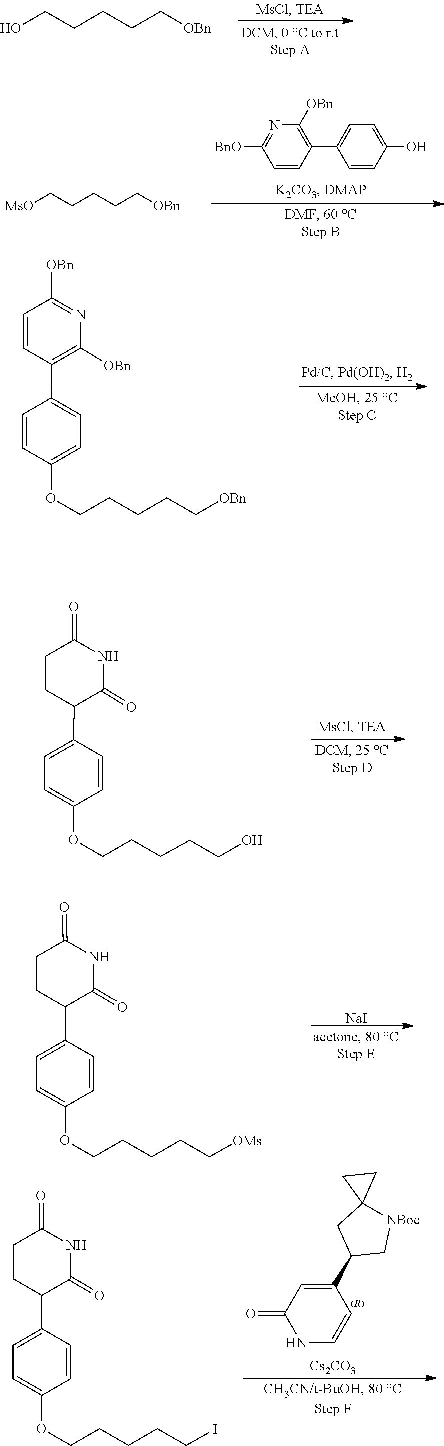

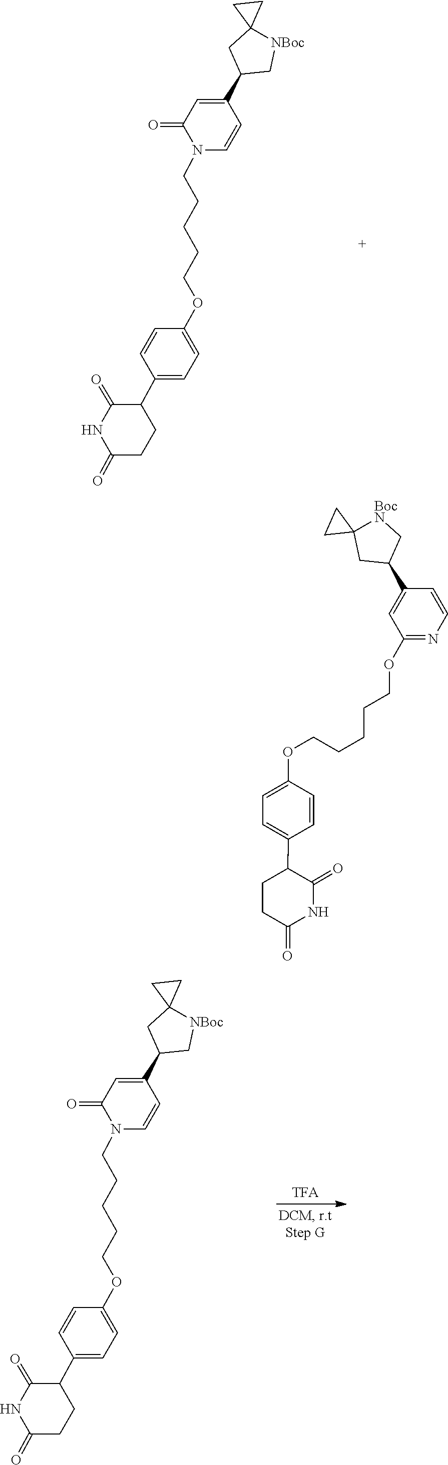 [SMILES: CC(C)(C)OC(=O)N1C[C@@H](c2ccn(CCCCCOc3ccc(C4CCC(=O)NC4=O)cc3)c(=O)c2)CC12CC2 ; CC(C)(C)OC(=O)N1C[C@@H](c2ccnc(OCCCCCOc3ccc(C4CCC(=O)NC4=O)cc3)c2)CC12CC2 ; CC(C)(C)OC(=O)N1C[C@@H](c2ccn(CCCCCOc3ccc(C4CCC(=O)NC4=O)cc3)c(=O)c2)CC12CC2]

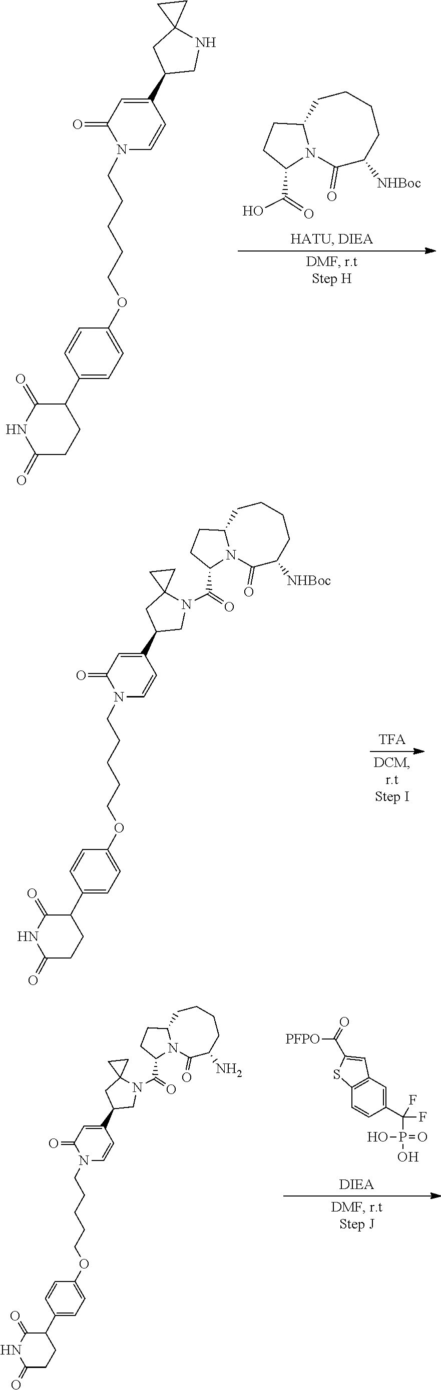

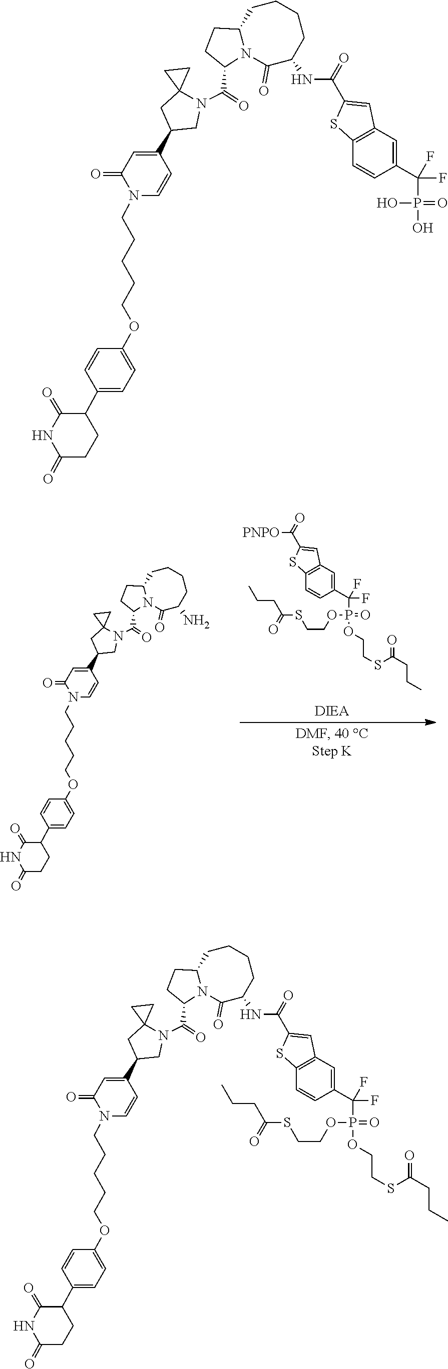

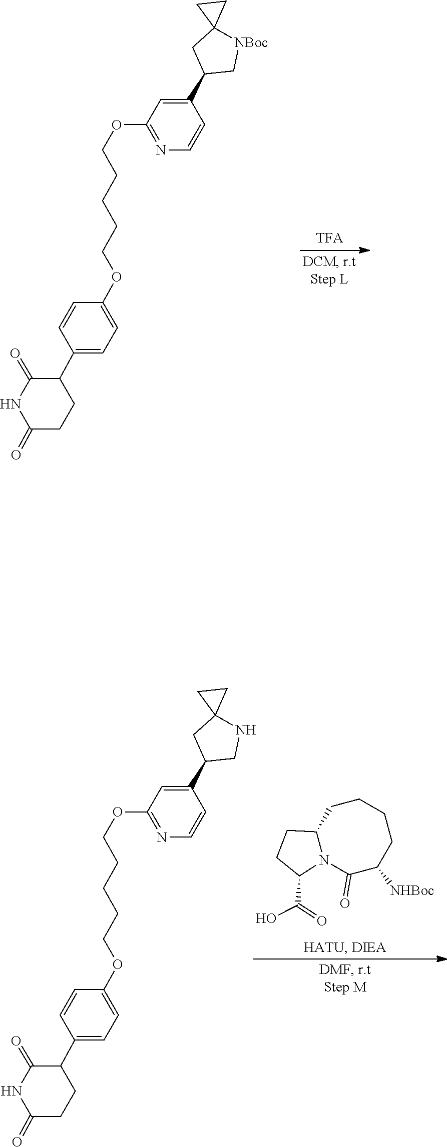 [SMILES: CC(C)(C)OC(=O)N1C[C@@H](c2ccnc(OCCCCCOc3ccc(C4CCC(=O)NC4=O)cc3)c2)CC12CC2 ; O=C1CCC(c2ccc(OCCCCCOc3cc([C@@H]4CNC5(CC5)C4)ccn3)cc2)C(=O)N1 ; CC(C)(C)OC(=O)N[C@H]1CCCC[C@H]2CC[C@@H](C(=O)O)N2C1=O]

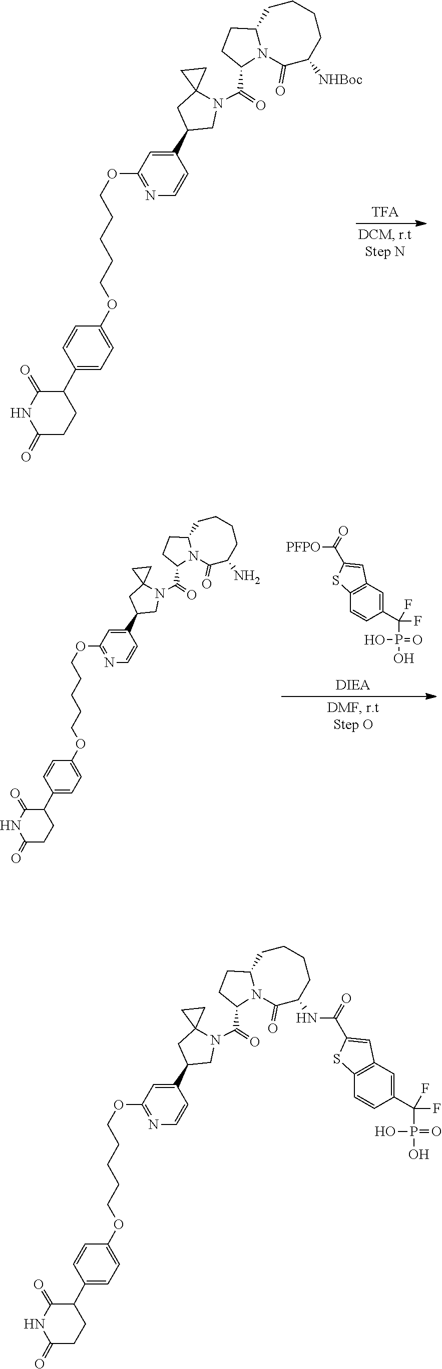

### STEP A: 5-(benzyloxy)pentyl methanesulfonate

To a solution of 5-(benzyloxy)pentan-1-ol (4 g, 20.6 mmol, 1 eq.) and TEA (4.2 g, 41.22 mmol, 2 eq.) in DCM (60 mL) was added MsCl (2.6 g, 22.8 mmol, 1.1 eq.) dropwise at 0° C. The reaction mixture was warmed up to room temperature and stirred for 0.5 hrs under N₂ atmosphere. After completion, the reaction mixture was diluted with water (100 mL) and extracted with DCM (100 mL×3). The combined organic layer was washed with brine (100 mL), dried over anhydrous Na₂SO₄ and concentrated under reduced pressure to give 5-(benzyloxy)pentyl methanesulfonate (5.5 g, quant.) as a colorless oil. LCMS (ESI) m/z=273 [M+H]⁺.

### STEP B: 2,6-bis(benzyloxy)-3-(4-((5-(benzyloxy)pentyl)oxy)phenyl)pyridine

To a solution of 5-(benzyloxy)pentyl methanesulfonate (3 g, 11.0 mmol, 1 eq.) and 4-(2,6-bis(benzyloxy)pyridin-3-yl)phenol (4.22 g, 11.03 mmol, 1.0 eq.) in DMF (100 mL) were added DMAP (134 mg, 1.10 mmol, 0.1 eq.) and K₂CO₃ (3.04 g, 22.1 mmol, 2 eq.). The reaction mixture was stirred at 60° C. for 48 hrs under N₂ atmosphere. After completion, the reaction mixture was diluted with water (100 mL) and extracted with EtOAc (100 mL×3). The combined organic layer was washed with brine (100 mL), dried over anhydrous Na₂SO₄ and concentrated under reduced pressure. The residue was purified by column chromatography on silica gel to give 2,6-bis(benzyloxy)-3-(4-((5-(benzyloxy)pentyl)oxy)phenyl)pyridine (3.95 g, 7.06 mmol, 64%) as a colorless oil. LCMS (ESI) m/z=560 [M+H]⁺.

### STEP C: 3-(4-((5-hydroxypentyl)oxy)phenyl)piperidine-2,6-dione

To a solution of 2,6-bis(benzyloxy)-3-(4-((5-(benzyloxy)pentyl)oxy)phenyl)pyridine (3.95 g, 7.06 mmol, 1 eq.) in MeOH (150 mL) were added Pd/C (400 mg) and Pd(OH)₂ (400 mg) at 25° C. under N₂. The suspension was degassed under vacuum and purged with H₂ several times. The resulting mixture was stirred at room temperature for 16 hrs. After completion, the suspension was filtered through a pad of Celite®, the filter cake was washed with MeOH (50 mL). The combined filtrates were concentrated to dryness to give 3-(4-((5-hydroxypentyl)oxy)phenyl)piperidine-2,6-dione (1.5 g, 5.15 mmol, 73%) as a white solid. LCMS (ESI) m/z=292 [M+H]⁺.

### STEP D: 5-(4-(2,6-dioxopiperidin-3-yl)phenoxy)pentyl methanesulfonate

To a solution of 3-(4-((5-hydroxypentyl)oxy)phenyl)piperidine-2,6-dione (1.5 g, 5.15 mmol, 1 eq.) in DCM (50 ml) were added TEA (1.0 g, 10.3 mmol, 2 eq.) and MsCl (587 mg, 5.66 mmol, 1.1 eq.). The reaction mixture was stirred at 25° C. for 1 hr under N₂ atmosphere. After completion, the reaction mixture was diluted with water (10 mL) and extracted with DCM (20 mL×3). The combined organic layer was washed with brine (10 mL), dried over anhydrous Na₂SO₄ and concentrated under reduced pressure to give 5-(4-(2,6-dioxopiperidin-3-yl)phenoxy)pentyl methanesulfonate (1.7 g, 4.61 mmol, 89%) as a white solid. LCMS (ESI) m/z=370 [M+H]⁺.

### STEP E: 3-(4-((5-iodopentyl)oxy)phenyl)piperidine-2,6-dione

To a solution of 5-(4-(2,6-dioxopiperidin-3-yl)phenoxy)pentyl methanesulfonate (1.7 g, 4.61 mmol, 1 eq.) in acetone (20 mL) was added NaI (2.1 g, 14.1 mmol, 3.0 eq.) at 25° C. The reaction mixture was stirred at 80° C. for 2 hrs under N₂ atmosphere. After completion, Na₂S₂O₃ in H₂O was added to the reaction solution until the yellow color faded away. The aqueous phase was extracted with EtOAc (10 mL×2). The combined organic layers were washed with brine (50 mL), dried over Na₂SO₄ and concentrated under reduced pressure to give crude product. The residue was purified by column chromatography on silica gel to give 3-(4-((5-iodopentyl)oxy)phenyl)piperidine-2,6-dione (1.5 g, 3.74 mmol, 82%) as a white solid. LCMS (ESI) m/z=402 [M+H]⁺.

### STEP F: tert-butyl (6R)-6-(1-(5-(4-(2,6-dioxopiperidin-3-yl)phenoxy)pentyl)-2-oxo-1,2-dihydropyridin-4-yl)-4-azaspiro[2.4]heptane-4-carboxylate and tert-butyl (6R)-6-(2-((5-(4-(2,6-dioxopiperidin-3-yl)phenoxy)pentyl)oxy)pyridin-4-yl)-4-azaspiro[2.4]heptane-4-carboxylate

To a solution of 3-(4-((5-iodopentyl)oxy)phenyl)piperidine-2,6-dione (1 g, 2.49 mmol, 1 eq.) and tert-butyl (R)-6-(2-oxo-1,2-dihydropyridin-4-yl)-4-azaspiro[2.4]heptane-4-carboxylate (723 mg, 2.49 mml, 1 eq.) in CH₃CN (20 mL) and t-BuOH (10 mL) was added and Cs₂CO₃ (2.04 g, 6.23 mmol, 2.5 eq.) at 25° C. The reaction mixture was stirred at 80° C. for 2 hrs under N₂ atmosphere. The mixture was purified by C18 column to afford tert-butyl (6R)-6-(1-(5-(4-(2,6-dioxopiperidin-3-yl)phenoxy)pentyl)-2-oxo-1,2-dihydropyridin-4-yl)-4-azaspiro[2.4]heptane-4-carboxylate (180 mg, 0.32 mmol, 13%) and tert-butyl (6R)-6-(2-((5-(4-(2,6-dioxopiperidin-3-yl)phenoxy)pentyl)oxy)pyridin-4-yl)-4-azaspiro[2.4]heptane-4-carboxylate (69 mg, 0.12 mmol, 5%) both isomers as white solid. LCMS (ESI) m/z=564 [M+H]⁺.

### STEP G: 3-(4-((5-(2-oxo-4-((R)-4-azaspiro[2.4]heptan-6-yl)pyridin-1(2H)-yl)pentyl)oxy)phenyl)piperidine-2,6-dione

To a solution of tert-butyl (6R)-6-(1-(5-(4-(2,6-dioxopiperidin-3-yl)phenoxy)pentyl)-2-oxo-1,2-dihydropyridin-4-yl)-4-azaspiro[2.4]heptane-4-carboxylate (90 mg, 0.16 mmol, 1 eq.) in DCM (2 mL) was added TFA (1 mL) and the resulting mixture was stirred at room temperature for 0.5 hrs. After completion, the reaction mixture was concentrated under reduced pressure to give 3-(4-((5-(2-oxo-4-((R)-4-azaspiro[2.4]heptan-6-yl)pyridin-1(2H)-yl)pentyl)oxy)phenyl)piperidine-2,6-dione (74 mg, quant.) as a yellow oil.

LCMS (ESI) m/z=464 [M+H]⁺.

### STEP H: tert-butyl ((3S,6S,10aS)-3-((6R)-6-(1-(5-(4-(2,6-dioxopiperidin-3-yl)phenoxy)pentyl)-2-oxo-1,2-dihydropyridin-4-yl)-4-azaspiro[2.4]heptane-4-carbonyl)-5-oxodecahydropyrrolo[1,2-a]azocin-6-yl)carbamate

A solution of (3S,6S,10aS)-6-((tert-butoxycarbonyl)amino)-5-oxodecahydropyrrolo[1,2-a]azocine-3-carboxylic acid (52 mg, 0.16 mmol, 1 eq.), DIEA (103 mg, 0.80 mmol, 5 eq.) and HATU (73 mg, 0.19 mmol, 1.2 eq.) in DMF (1 mL) was stirred at room temperature for 20 min. Then a solution of 3-(4-((5-(2-oxo-4-((R)-4-azaspiro[2.4]heptan-6-yl)pyridin-1(2H)-yl)pentyl)oxy)phenyl)piperidine-2,6-dione (74 mg, 0.16 mmol, 1 eq.) in DMF (1 mL) was added. The reaction mixture was stirred at room temperature for 1 hr. After completion, the reaction mixture was purified by C18 column to afford tert-butyl ((3S,6S,10aS)-3-((6R)-6-(1-(5-(4-(2,6-dioxopiperidin-3-yl)phenoxy)pentyl)-2-oxo-1,2-dihydropyridin-4-yl)-4-azaspiro[2.4]heptane-4-carbonyl)-5-oxodecahydropyrrolo[1,2-a]azocin-6-yl)carbamate (112 mg, 0.14 mmol, 91%) as a white solid. LCMS (ESI) m/z=772 [M+H]⁺.

### STEP I: 3-(4-((5-(4-((R)-4-((3S,6S,10aS)-6-amino-5-oxodecahydropyrrolo[1,2-a]azocine-3-carbonyl)-4-azaspiro[2.4]heptan-6-yl)-2-oxopyridin-1(2H)-yl)pentyl)oxy)phenyl)piperidine-2,6-dione

To a solution of tert-butyl ((3S,6S,10aS)-3-((6R)-6-(1-(5-(4-(2,6-dioxopiperidin-3-yl)phenoxy)pentyl)-2-oxo-1,2-dihydropyridin-4-yl)-4-azaspiro[2.4]heptane-4-carbonyl)-5-oxodecahydropyrrolo[1,2-a]azocin-6-yl)carbamate (112 mg, 0.14 mmol) in DCM (3 mL) was added TFA (1 mL). The reaction mixture was stirred at room temperature for 0.5 hrs. The reaction mixture was concentrated under reduce pressure to give 3-(4-((5-(4-((R)-4-((3S,6S,10aS)-6-amino-5-oxodecahydropyrrolo[1,2-a]azocine-3-carbonyl)-4-azaspiro[2.4]heptan-6-yl)-2-oxopyridin-1(2H)-yl)pentyl)oxy)phenyl)piperidine-2,6-dione (94 mg, quant.) (TFA salt) as a yellow oil, which was used in next step directly without further purification. LCMS (ESI) m/z=672 [M+H]⁺.

### STEP J. ((2-(((3S,6S,10aS)-3-((6R)-6-(1-(5-(4-(2,6-dioxopiperidin-3-yl)phenoxy)pentyl)-2-oxo-1,2-dihydropyridin-4-yl)-4-azaspiro[2.4]heptane-4-carbonyl)-5-oxodecahydropyrrolo[1,2-a]azocin-6-yl)carbamoyl)benzo[b]thiophen-5-yl)difluoromethyl)phosphonic acid

To a solution of 3-(4-((5-(4-((R)-4-((3S,6S,10aS)-6-amino-5-oxodecahydropyrrolo[1,2-a]azocine-3-carbonyl)-4-azaspiro[2.4]heptan-6-yl)-2-oxopyridin-1(2H)-yl)pentyl)oxy)phenyl)piperidine-2,6-dione (47 mg, 70 μmol, 1.0 eq.) in DMF (2 mL) were added DIEA (90 mg, 0.70 mmol, 10 eq.) and (difluoro(2-((perfluorophenoxy)carbonyl)benzo[b]thiophen-5-yl)methyl)phosphonic acid (32 mg, 67 μmol, 0.95 eq). The reaction mixture was stirred at room temperature for 0.5 hrs. After completion, the reaction mixture was purified by prep-HPLC to afford ((2-(((3S,6S,10aS)-3-((6R)-6-(1-(5-(4-(2,6-dioxopiperidin-3-yl)phenoxy)pentyl)-2-oxo-1,2-dihydropyridin-4-yl)-4-azaspiro[2.4]heptane-4-carbonyl)-5-oxodecahydropyrrolo[1,2-a]azocin-6-yl)carbamoyl)benzo[b]thiophen-5-yl)difluoromethyl)phosphonic acid (Compound 17, 32.7 mg, 34 μmol, 49%) as a white solid.

¹H NMR (400 MHz, DMSO-d6) δ 10.78 (s, 1H), 8.91-8.80 (m, 1H), 8.31 (s, 1H), 8.14-8.03 (m, 2H), 7.63-7.53 (m, 2H), 7.12-7.05 (m, 2H), 6.88-6.80 (m, 2H), 6.32 (s, 1H), 6.24-6.18 (m, 1H), 4.98-4.89 (m, 1H), 4.51-4.47 (m, 1H), 4.24-4.21 (m, 1H), 4.07-4.04 (m, 1H), 3.91-3.88 (m, 2H), 3.84-3.80 (m, 2H), 3.77-3.74 (m, 1H), 3.39-3.32 (m, 1H), 2.70-2.59 (m, 1H), 2.35-1.20 (m, 26H), 0.47 (s, 2H).

LCMS (ESI) m/z=962.3 [M+H]⁺.

### STEP K: S,S(((((2-(((3S,6S,10aS)-3-((6R)-6-(1-(5-(4-(2,6-dioxopiperidin-3-yl)phenoxy)pentyl)-2-oxo-1,2-dihydropyridin-4-yl)-4-azaspiro[2.4]heptane-4-carbonyl)-5-oxodecahydropyrrolo[1,2-a]azocin-6-yl)carbamoyl)benzo[b]thiophen-5-yl)difluoromethyl)phosphoryl)bis(oxy))bis(ethane-2,1-diyl)) dibutanethioate

To a solution of 4-nitrophenyl 5-((bis(2-(butyrylthio)ethoxy)phosphoryl)difluoromethyl)benzo[b]thiophene-2-carboxylate (45 mg, 67 μmol, 0.95 eq.) in DMF (1 ml) were added DIEA (45 mg, 350 μmol, 5 eq.) and 3-(4-((5-(4-((R)-4-((3S,6S,10aS)-6-amino-5-oxodecahydropyrrolo[1,2-a]azocine-3-carbonyl)-4-azaspiro[2.4]heptan-6-yl)-2-oxopyridin-1(2H)-yl)pentyl)oxy)phenyl)piperidine-2,6-dione (47 mg, 70 μmol, 1.0 eq.). The reaction mixture was stirred at 40° C. for 16 hrs under N₂ atmosphere. The mixture was purified by prep-HPLC to afford S,S [symbol] (((((2-(((3S,6S,10aS)-3-((6R)-6-(1-(5-(4-(2,6-dioxopiperidin-3-yl)phenoxy)pentyl)-2-oxo-1,2-dihydropyridin-4-yl)-4-azaspiro[2.4]heptane-4-carbonyl)-5-oxodecahydropyrrolo[1,2-a]azocin-6-yl)carbamoyl)benzo[b]thiophen-5-yl)difluoromethyl)phosphoryl)bis(oxy))bis(ethane-2,1-diyl)) dibutanethioate (Compound 18, 18.1 mg, 15 μmol, 21%) as a white solid.

¹H NMR (400 MHz, DMSO-d6) δ 10.79 (s, 1H), 8.97-8.87 (m, 1H), 8.35 (s, 1H), 8.23-8.11 (m, 2H), 7.65-7.53 (m, 2H), 7.18-7.03 (m, 2H), 6.91-6.76 (m, 2H), 6.32 (s, 1H), 6.24-6.16 (m, 1H), 5.03-4.86 (m, 1H), 4.56-4.41 (m, 1H), 4.30-4.02 (m, 6H), 3.97-3.66 (m, 6H), 3.19-3.07 (m, 4H), 2.70-2.59 (m, 1H), 2.56-2.51 (m, 4H), 2.49-2.41 (m, 1H), 2.32-1.20 (m, 29H), 0.92-0.79 (m, 6H), 0.47 (s, 2H).

LCMS (ESI) m/z=1222.4 [M+H]⁺.

### STEP L: 3-(4-((5-((4-((R)-4-azaspiro[2.4]heptan-6-yl)pyridin-2-yl)oxy)pentyl)oxy)phenyl)piperidine-2,6-dione

To a solution of tert-butyl (6R)-6-(2-((5-(4-(2,6-dioxopiperidin-3-yl)phenoxy)pentyl)oxy)pyridin-4-yl)-4-azaspiro[2.4]heptane-4-carboxylate (69 mg, 0.12 mmol, 1 eq.) in DCM (2 ml) was added TFA (1 ml). The reaction mixture was stirred at room temperature for 0.5 hrs. After completion, the reaction mixture was concentrated under reduced pressure to give 3-(4-((5-((4-((R)-4-azaspiro[2.4]heptan-6-yl)pyridin-2-yl)oxy)pentyl)oxy)phenyl)piperidine-2,6-dione (57 mg, quant.) as a yellow oil, which was used in next step directly without further purification. LCMS (ESI) m/z=464 [M+H]⁺.

### STEP M: tert-butyl ((3S,6S,10aS)-3-((6R)-6-(2-((5-(4-(2,6-dioxopiperidin-3-yl)phenoxy)pentyl)oxy)pyridin-4-yl)-4-azaspiro[2.4]heptane-4-carbonyl)-5-oxodecahydropyrrolo[1,2-a]azocin-6-yl)carbamate

A solution of (3S,6S,10aS)-6-((tert-butoxycarbonyl)amino)-5-oxodecahydropyrrolo[1,2-a]azocine-3-carboxylic acid (40 mg, 0.12 mmol, 1 eq.), DIEA (79 mg, 0.61 mmol, 5 eq.) and HATU (56 mg, 0.15 mmol, 1.2 eq) in DMF (1 ml) was stirred at room temperature for 20 min. Then a solution of 3-(4-((5-((4-((R)-4-azaspiro[2.4]heptan-6-yl)pyridin-2-yl)oxy)pentyl)oxy)phenyl)piperidine-2,6-dione (57 mg, 0.12 mmol, 1 eq.) in DMF (1 ml) was added. The reaction mixture was stirred at room temperature for 1 hr. The reaction mixture was purified by C18 column to afford tert-butyl ((3S,6S,10aS)-3-((6R)-6-(2-((5-(4-(2,6-dioxopiperidin-3-yl)phenoxy)pentyl)oxy)pyridin-4-yl)-4-azaspiro[2.4]heptane-4-carbonyl)-5-oxodecahydropyrrolo[1,2-a]azocin-6-yl)carbamate (43 mg, 56 μmol, 46%) as a white solid. LCMS (ESI) m/z=772 [M+H]⁺.

### STEP N: 3-(4-((5-((4-((R)-4-((3S,6S,10aS)-6-amino-5-oxodecahydropyrrolo[1,2-a]azocine-3-carbonyl)-4-azaspiro[2.4]heptan-6-yl)pyridin-2-yl)oxy)pentyl)oxy)phenyl)piperidine-2,6-dione

To a solution of tert-butyl ((3S,6S,10aS)-3-((6R)-6-(2-((5-(4-(2,6-dioxopiperidin-3-yl)phenoxy)pentyl)oxy)pyridin-4-yl)-4-azaspiro[2.4]heptane-4-carbonyl)-5-oxodecahydropyrrolo[1,2-a]azocin-6-yl)carbamate (43 mg, 56 μmol, 1 eq.) in DCM (3 mL) was added TFA (1 mL). The reaction mixture was stirred at room temperature for 0.5 hrs. After completion, the reaction mixture was concentrated under reduce pressure to give 3-(4-((5-((4-((R)-4-((3S,6S,10aS)-6-amino-5-oxodecahydropyrrolo[1,2-a]azocine-3-carbonyl)-4-azaspiro[2.4]heptan-6-yl)pyridin-2-yl)oxy)pentyl)oxy)phenyl)piperidine-2,6-dione (34 mg, quant.) as a yellow oil, which was used in next step directly without further purification.

LCMS (ESI) m/z=672 [M+H]⁺.

### STEP O: ((2-(((3S,6S,10aS)-3-((6R)-6-(2-((5-(4-(2,6-dioxopiperidin-3-yl)phenoxy)pentyl)oxy)pyridin-4-yl)-4-azaspiro[2.4]heptane-4-carbonyl)-5-oxodecahydropyrrolo[1,2-a]azocin-6-yl)carbamoyl)benzo[b]thiophen-5-yl)difluoromethyl)phosphonic acid

To a solution of 3-(4-((5-(4-((R)-4-((3S,6S,10aS)-6-amino-5-oxodecahydropyrrolo[1,2-a]azocine-3-carbonyl)-4-azaspiro[2.4]heptan-6-yl)-2-oxopyridin-1(2H)-yl)pentyl)oxy)phenyl)piperidine-2,6-dione (34 mg, 51 μmol, 1.0 eq.) and DIEA (65 mg, 0.50 mmol, 10 eq.) in DMF (2 mL) was added (difluoro(2-((perfluorophenoxy)carbonyl)benzo[b]thiophen-5-yl)methyl)phosphonic acid (22 mg, 47 μmol, 0.95 eq.). The reaction mixture was stirred at room temperature for 0.5 hrs. After completion, the reaction mixture was purified by prep-HPLC to afford ((2-(((3S,6S,10aS)-3-((6R)-6-(2-((5-(4-(2,6-dioxopiperidin-3-yl)phenoxy)pentyl)oxy)pyridin-4-yl)-4-azaspiro[2.4]heptane-4-carbonyl)-5-oxodecahydropyrrolo[1,2-a]azocin-6-yl)carbamoyl)benzo[b]thiophen-5-yl)difluoromethyl)phosphonic acid (TFA salt) (Compound 19, 20.8 mg, 22 μmol, 43%) as a white solid.

¹H NMR (400 MHz, DMSO-d6) δ 10.78 (s, 1H), 8.89-8.80 (m, 1H), 8.31 (s, 1H), 8.15-8.02 (m, 3H), 7.62-7.55 (m, 1H), 7.13-7.07 (m, 2H), 7.00-6.95 (m, 1H), 6.88-6.77 (m, 3H), 4.96-4.93 (m, 1H), 4.53-4.48 (m, 1H), 4.27-4.21 (m, 3H), 4.16-4.09 (m, 1H), 3.95-3.88 (m, 2H), 3.81-3.72 (m, 2H), 3.57-3.47 (m, 1H), 2.68-2.60 (m, 1H), 2.40-1.38 (m, 25H), 0.61-0.32 (m, 2H).

LCMS (ESI) m/z=962.3 [M+H]⁺.

### Preparation of S,S(((((2-(((3S,6S,9aS)-3-(3-(4-((2-(4-(2,6-dioxopiperidin-3-yl)phenoxy)ethyl)(methyl)amino)pyridin-3-yl)azetidine-1-carbonyl)-5-oxooctahydro-1H-pyrrolo[1,2-a]azepin-6-yl)carbamoyl)benzo[b]thiophen-5-yl)difluoromethyl)phosphoryl)bis(oxy))bis(ethane-2,1-diyl)) dibutanethioate (Compound 20) and ((2-(((3S,6S,9aS)-3-(3-(4-((2-(4-(2,6-dioxopiperidin-3-yl)phenoxy)ethyl)(methyl)amino)pyridin-3-yl)azetidine-1-carbonyl)-5-oxooctahydro-1H-pyrrolo[1,2-a]azepin-6-yl)carbamoyl)benzo[b]thiophen-5-yl)difluoromethyl)phosphonic acid (Compound 21)

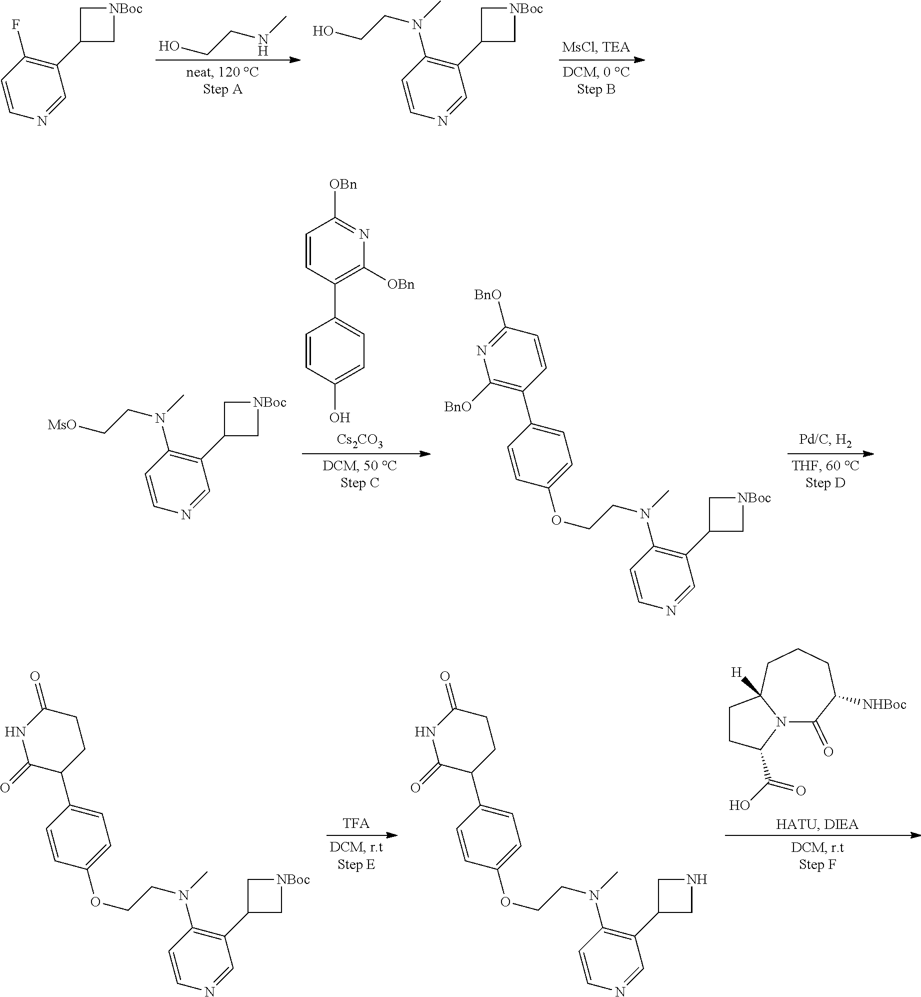

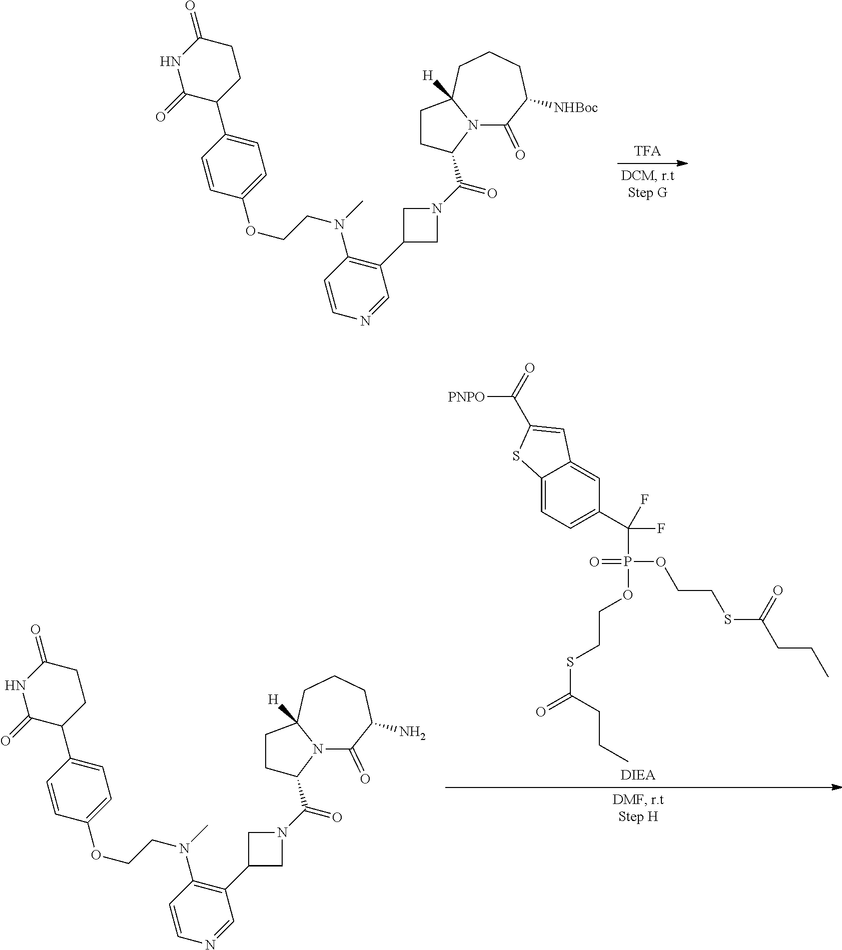

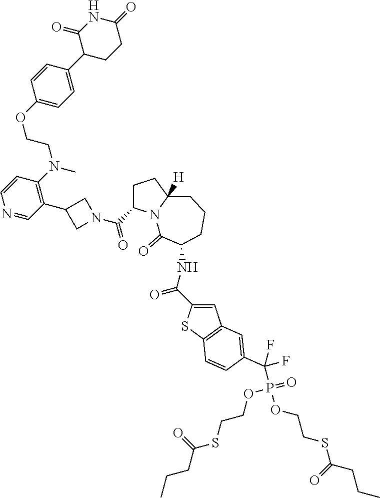 [SMILES: CCCC(=O)SCCOP(=O)(OCCSC(=O)CCC)C(F)(F)c1ccc2sc(C(=O)N[C@H]3CCC[C@H]4CC[C@@H](C(=O)N5CC(c6cnccc6N(C)CCOc6ccc(C7CCC(=O)NC7=O)cc6)C5)N4C3=O)cc2c1]

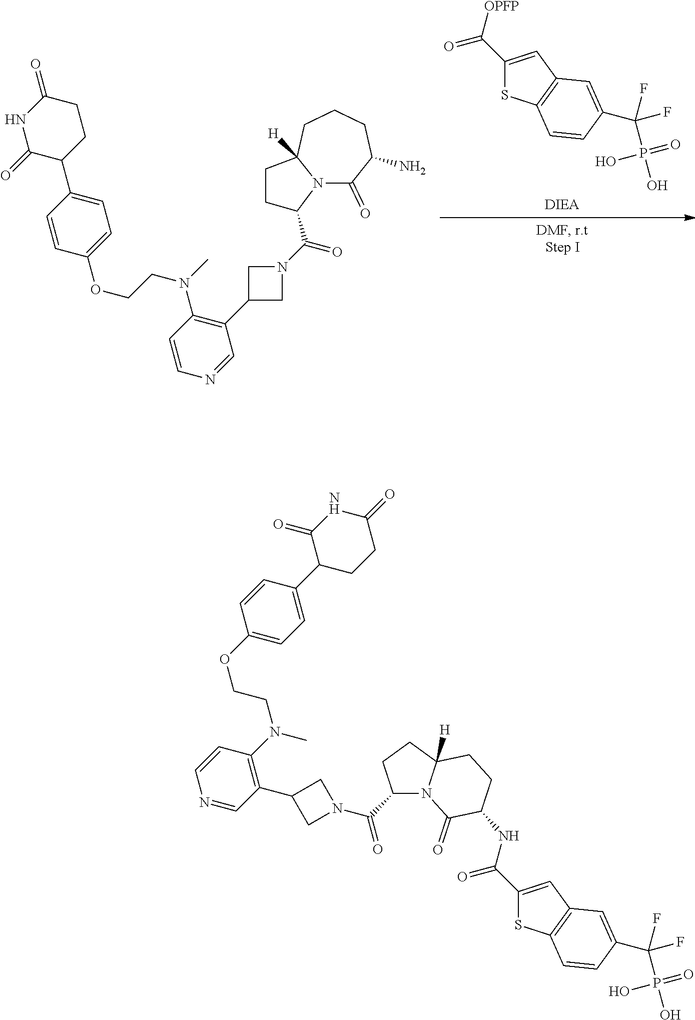

### STEP A: tert-butyl 3-(4-((2-hydroxyethyl)(methyl)amino)pyridin-3-yl)azetidine-1-carboxylate

2-Methylpropan-2-yl 3-(4-fluoropyridin-3-yl) azetidine-1-carboxylate (500 mg, 1.98 mmol, 1.0 eq) was added to a solution of 2-(methylamino) ethan-1-ol (1.5 g, 19.8 mmol, 10.0 eq.) at 25° C. The resulting mixture was heated to 120° C. and stirred for 12 hrs. After completion, the reaction mixture was purified by Biotage® C18 column chromatography to get tert-butyl 3-(4-((2-hydroxyethyl)(methyl)amino)pyridin-3-yl)azetidine-1-carboxylate (550 mg, 1.79 mmol, 90%) as a yellow oil. LCMS (ESI): m/z=308 [M+H]⁺.

¹H NMR (400 MHz, CDCl₃) δ 8.51 (s, 1H), 8.31 (d, J=5.6 Hz, 1H), 6.88 (d, J=5.6 Hz, 1H), 4.34 (t, J=8.1 Hz, 2H), 4.11-3.97 (m, 3H), 3.79 (t, J=5.7 Hz, 2H), 3.13 (t, J=5.7 Hz, 2H), 2.78 (s, 3H), 2.57 (br, 1H), 1.46 (s, 9H).

### STEP B: tert-butyl 3-(4-(methyl(2-((methylsulfonyl)oxy)ethyl)amino)pyridin-3-yl)azetidine-1-carboxylate

To a solution of tert-butyl 3-(4-((2-hydroxyethyl)(methyl)amino)pyridin-3-yl)azetidine-1-carboxylate (550 mg, 1.79 mmol, 1.0 eq.) in DCM (10 mL) were added TEA (0.5 mL, 3.58 mmol, 2.0 eq.) and MsCl (246 mg, 2.15 mmol, 1.2 eq.) at 0° C. and the resulting mixture was stirred at 0° C. for 1 hr. After completion, the reaction mixture was diluted with H₂O (10 mL) and extracted with DCM (20 mL×2). The combined organic layers was washed with brine (10 mL), dried over anhydrous Na₂SO₄ and concentrated under reduced pressure to afford crude tert-butyl 3-(4-(methyl(2-((methylsulfonyl)oxy)ethyl)amino)pyridin-3-yl)azetidine-1-carboxylate (550 mg, quant.) as a yellow oil, which was used in next step directly without further purification. LCMS (ESI): m/z=386 [M+H]⁺.

### STEP C: tert-butyl 3-(4-((2-(4-(2,6-bis(benzyloxy)pyridin-3-yl)phenoxy)ethyl)(methyl)amino)pyridin-3-yl)azetidine-1-carboxylate

To a solution of tert-butyl 3-(4-(methyl(2-((methylsulfonyl)oxy)ethyl)amino)pyridin-3-yl)azetidine-1-carboxylate (550 mg, 1.43 mmol, 1.0 eq) in DMF (10 mL) were added 4-(2,6-bis(benzyloxy)pyridin-3-yl)phenol (438 mg, 1.14 mmol, 0.8 eq.) and Cs₂CO₃ (930 mg, 2.85 mmol, 2.0 eq.) at 25° C. The mixture was heated to 50° C. and stirred for 12 hrs. After completion, the reaction mixture was diluted with H₂O (10 mL) and extracted with EtOAc (20 mL×2). The combined organic layers was washed with brine (10 mL), dried over anhydrous Na₂SO₄ and concentrated under reduced pressure. The residue was purified by flash column chromatography on silica gel to afford tert-butyl 3-(4-((2-(4-(2,6-bis(benzyloxy)pyridin-3-yl)phenoxy)ethyl)(methyl)amino)pyridin-3-yl)azetidine-1-carboxylate (550 mg, 0.82 mmol, 57%) as a yellow oil. LCMS (ESI): m/z=673 [M+H]⁺.

### STEP D: tert-butyl 3-(4-((2-(4-(2,6-dioxopiperidin-3-yl)phenoxy)ethyl)(methyl)amino)pyridin-3-yl)azetidine-1-carboxylate

To a solution of tert-butyl 3-(4-((2-(4-(2,6-bis(benzyloxy)pyridin-3-yl)phenoxy)ethyl)(methyl)amino)pyridin-3-yl)azetidine-1-carboxylate (400 mg, 0.60 mmol 1.0 eq.) in THF (30 mL) was added Pd/C (50 mg) under nitrogen. The suspension was degassed under vacuum and purged with H₂ several times. The resulting mixture was heated to 60° C. and stirred for 12 hrs under H₂ (15 Psi). After completion, the suspension was filtered through a pad of Celite®, the filter cake was washed with THF (40 mL). The combined filtrates were concentrated under reduced pressure. The residue was purified by Biotage® C18 column chromatography to give tert-butyl 3-(4-((2-(4-(2,6-dioxopiperidin-3-yl)phenoxy)ethyl)(methyl)amino)pyridin-3-yl)azetidine-1-carboxylate (220 mg, 0.45 mmol, 75%) as a yellow oil. LCMS (ESI): m/z=495 [M+H]⁺.

¹H NMR (400 MHz, CDCl₃) δ 8.49 (s, 1H), 8.34 (d, J=5.6 Hz, 1H), 7.98 (s, 1H), 7.11 (d, J=8.6 Hz, 2H), 6.88 (d, J=5.6 Hz, 1H), 6.80 (d, J=8.7 Hz, 2H), 4.38-4.32 (m, 2H), 4.12-4.01 (m, 5H), 3.75-3.69 (m, 1H), 3.41-3.35 (m, 2H), 2.85 (s, 3H), 2.79-2.61 (m, 2H), 2.31-2.16 (m, 2H), 1.46 (s, 9H).

### STEP E: 3-(4-(2-((3-(azetidin-3-yl)pyridin-4-yl)(methyl)amino)ethoxy)phenyl)piperidine-2,6-dione

To a solution of tert-butyl 3-(4-((2-(4-(2,6-dioxopiperidin-3-yl)phenoxy)ethyl)(methyl)amino)pyridin-3-yl)azetidine-1-carboxylate (100 mg, 0.20 mmol, 1.0 eq.) in dry DCM (6 mL) was added TFA (2 mL) at 25° C. The mixture was stirred at 25° C. for 30 min. After completion, the reaction mixture was concentrated under reduced pressure to give crude (3-{4-[(2-{[3-(azetidin-3-yl)pyridin-4-yl](methyl)amino}ethyl)oxy]phenyl}hexahydropyridine-2,6-dione (80 mg, quant.) as a yellow oil, which was used in next step directly without further purification. LCMS (ESI): m/z=395 [M+H]⁺.

### STEP F: tert-butyl ((3S,6S,9aS)-3-(3-(4-((2-(4-(2,6-dioxopiperidin-3-yl)phenoxy)ethyl)(methyl)amino)pyridin-3-yl)azetidine-1-carbonyl)-5-oxooctahydro-1H-pyrrolo[1,2-a]azepin-6-yl)carbamate

To a mixture of (3S,6S,9aS)-6-((tert-butoxycarbonyl)amino)-5-oxooctahydro-1H-pyrrolo[1,2-a]azepine-3-carboxylic acid (75 mg, 0.24 mmol, 1.1 eq.) and HATU (92 mg, 0.24 mmol, 1.1 eq.) in DMF (1 mL) were added DIEA (85 mg, 0.66 mmol, 5.0 eq.) and 3-(4-(2-((3-(azetidin-3-yl)pyridin-4-yl)(methyl)amino)ethoxy)phenyl)piperidine-2,6-dione (87 mg, 0.22 mmol, 1.0 eq.). The resulting mixture was stirred at room temperature for 30 min. After completion, the reaction mixture was purified directly by Biotage® C18 column chromatography to afford tert-butyl ((3S,6S,9aS)-3-(3-(4-((2-(4-(2,6-dioxopiperidin-3-yl)phenoxy)ethyl)(methyl)amino)pyridin-3-yl)azetidine-1-carbonyl)-5-oxooctahydro-1H-pyrrolo[1,2-a]azepin-6-yl)carbamate (130 mg, 0.21 mmol, 85%) as a white solid. LCMS (ESI): m/z=689.7 [M+H]⁺.

### STEP G: 3-(4-(2-((3-(1-((3S,6S,9aS)-6-amino-5-oxooctahydro-1H-pyrrolo[1,2-a]azepine-3-carbonyl)azetidin-3-yl)pyridin-4-yl)(methyl)amino)ethoxy)phenyl)piperidine-2,6-dione

To a solution of tert-butyl ((3S,6S,9aS)-3-(3-(4-((2-(4-(2,6-dioxopiperidin-3-yl)phenoxy)ethyl)(methyl)amino)pyridin-3-yl)azetidine-1-carbonyl)-5-oxooctahydro-1H-pyrrolo[1,2-a]azepin-6-yl)carbamate (65 mg, 94 μmol, 1.0 eq.) in dry DCM (4 mL) was added TFA (1 mL) at room temperature. The mixture was stirred at room temperature for 2 hrs. After completion, the reaction mixture was concentrated under reduced pressure to give 3-(4-(2-((3-(1-((3S,6S,9aS)-6-amino-5-oxooctahydro-1H-pyrrolo[1,2-a]azepine-3-carbonyl)azetidin-3-yl)pyridin-4-yl)(methyl)amino)ethoxy)phenyl)piperidine-2,6-dione (50 mg, quant.) as a white solid, which was used in next step without further purification. LCMS (ESI): m/z=589.4 [M+H]⁺.

### STEP H: S,S(((((2-(((3S,6S,9aS)-3-(3-(4-((2-(4-(2,6-dioxopiperidin-3-yl)phenoxy)ethyl)(methyl)amino)pyridin-3-yl)azetidine-1-carbonyl)-5-oxooctahydro-1H-pyrrolo[1,2-a]azepin-6-yl)carbamoyl)benzo[b]thiophen-5-yl)difluoromethyl)phosphoryl)bis(oxy))bis(ethane-2,I-diyl)) dibutanethioate

To a mixture of 3-(4-(2-((3-(1-((3S,6S,9aS)-6-amino-5-oxooctahydro-1H-pyrrolo[1,2-a]azepine-3-carbonyl)azetidin-3-yl)pyridin-4-yl)(methyl)amino)ethoxy)phenyl)piperidine-2,6-dione (50 mg, 85 μmol, 1.0 eq.) and DIEA (33 mg, 0.25 mmol, 3.0 eq.) in DMF (1 mL) was added 4-nitrophenyl 5-((bis(2-(butyrylthio)ethoxy)phosphoryl)difluoromethyl)benzo[b]thiophene-2-carboxylate (64 mg, 93 μmol, 1.1 eq.). The mixture was stirred at room temperature for 30 min. After completion, the reaction mixture was purified by prep-HPLC to give S,S [symbol] (((((2-(((3S,6S,9aS)-3-(3-(4-((2-(4-(2,6-dioxopiperidin-3-yl)phenoxy)ethyl)(methyl)amino)pyridin-3-yl)azetidine-1-carbonyl)-5-oxooctahydro-1H-pyrrolo[1,2-a]azepin-6-yl)carbamoyl)benzo[b]thiophen-5-yl)difluoromethyl)phosphoryl)bis(oxy))bis(ethane-2,1-diyl)) dibutanethioate (TFA salt) (Compound 20, 14.4 mg, 11 μmol, 13%) as a white solid.

¹H NMR (400 MHz, DMSO-d6) δ 10.78 (s, 1H), 8.88-8.72 (m, 1H), 8.40-8.25 (m, 3H), 8.24-8.11 (m, 2H), 7.59 (d, J=5.7 Hz, 1H), 7.30-7.21 (m, 1H), 7.07 (d, J=8.5 Hz, 2H), 6.76 (d, J=8.0 Hz, 2H), 4.79-4.58 (m, 2H), 4.43-4.25 (m, 3H), 4.23-4.09 (m, 7H), 4.02-3.72 (m, 5H), 3.14 (s, 7H), 2.68-2.59 (m, 1H), 2.58-2.52 (m, 4H), 2.44 (s, 1H), 2.23-2.07 (m, 2H), 2.04-1.91 (m, 3H), 1.90-1.65 (m, 7H), 1.60-1.50 (m, 4H), 0.89-0.83 (m, 6H).

LCMS (ESI): m/z=1139.4 [M+H]⁺.

### STEP I: ((2-(((3S,6S,9aS)-3-(3-(4-((2-(4-(2,6-dioxopiperidin-3-yl)phenoxy)ethyl)(methyl)amino)pyridin-3-yl)azetidine-1-carbonyl)-5-oxooctahydro-1H-pyrrolo[1,2-a]azepin-6-yl)carbamoyl)benzo[b]thiophen-5-yl)difluoromethyl)phosphonic acid

To a mixture of (2S,4R)-1-((S)-2-((4-((3-(1-((3S,6S,9aS)-6-amino-5-oxooctahydro-1H-pyrrolo[1,2-a]azepine-3-carbonyl)azetidin-3-yl)pyridin-4-yl)oxy)butyl)amino)-3,3-dimethylbutanoyl)-4-hydroxy-N-(4-(4-methylthiazol-5-yl)benzyl)pyrrolidine-2-carboxamide (50 mg, 85 μmol, 1.0 eq.) and DIEA (33 mg, 0.25 mmol, 3.0 eq.) in DMF (1 mL) was added (difluoro(2-((perfluorophenoxy)carbonyl)benzo[b]thiophen-5-yl)methyl)phosphonic acid (39.5 mg, 102 μmol, 1.2 eq.). The mixture was stirred at 25° C. for 30 min. After completion, the reaction mixture was purified by prep-HPLC to give ((2-(((3S,6S,9aS)-3-(3-(4-((2-(4-(2,6-dioxopiperidin-3-yl)phenoxy)ethyl)(methyl)amino)pyridin-3-yl)azetidine-1-carbonyl)-5-oxooctahydro-1H-pyrrolo[1,2-a]azepin-6-yl)carbamoyl)benzo[b]thiophen-5-yl)difluoromethyl)phosphonic acid (TFA salt) (Compound 21, 16.3 mg, 18 μmol, 21%) as a white solid.

¹H NMR (400 MHz, DMSO-d6) δ 10.78 (s, 1H), 8.74 (dd, J=33.6, 7.4 Hz, 1H), 8.38-8.25 (m, 3H), 8.16-8.05 (m, 2H), 7.62-7.56 (m, 1H), 7.28-7.21 (m, 1H), 7.07 (d, J=8.5 Hz, 2H), 6.75 (d, J=7.4 Hz, 2H), 4.79-4.60 (m, 2H), 4.40-4.30 (m, 2H), 4.25-4.14 (m, 4H), 4.01-3.81 (m, 4H), 3.77-3.72 (m, 1H), 3.13 (s, 3H), 2.68-2.59 (m, 1H), 2.49-2.42 (m, 1H), 2.22-2.07 (m, 2H), 2.03-1.67 (m, 10H).

LCMS (ESI): m/z=879.5 [M+H]⁺.

### Preparation of ((2-(((3S,6S,10aS)-3-((rel-3S,4R)-3-cyano-4-(3-(4-(1-(2,6-dioxopiperidin-3-yl)-2-oxo-2,3-dihydro-1H-benzo[d]imidazol-5-yl)piperazin-1-yl)phenyl)pyrrolidine-1-carbonyl)-5-oxodecahydropyrrolo[1,2-a]azocin-6-yl)carbamoyl)benzo[b]thiophen-5-yl)difluoromethyl)phosphonic acid (Compound 22) and S,S(((((2-(((3S,6S,10aS)-3-((rel-3S,4R)-3-cyano-4-(3-(4-(1-(2,6-dioxopiperidin-3-yl)-2-oxo-2,3-dihydro-1H-benzo[d]imidazol-5-yl)piperazin-1-yl)phenyl)pyrrolidine-1-carbonyl)-5-oxodecahydropyrrolo[1,2-a]azocin-6-yl)carbamoyl)benzo[b]thiophen-5-yl)difluoromethyl)phosphoryl)bis(oxy))bis(ethane-2,1-diyl)) dibutanethioate (Compound 23)

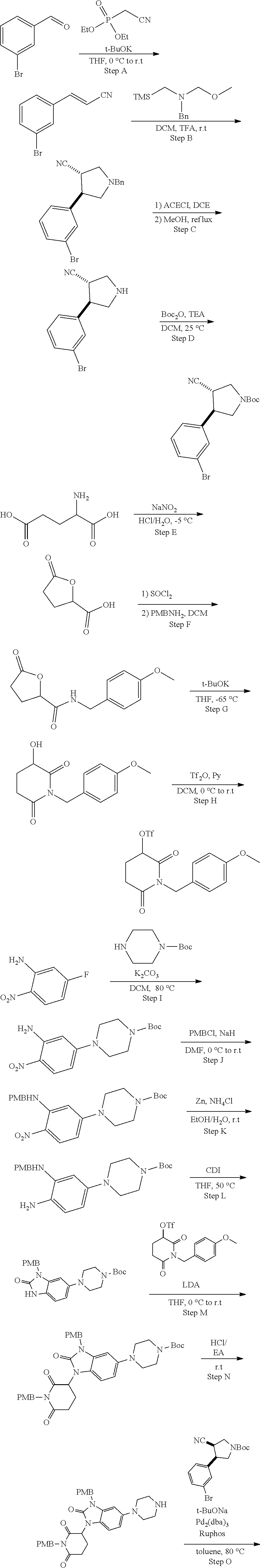

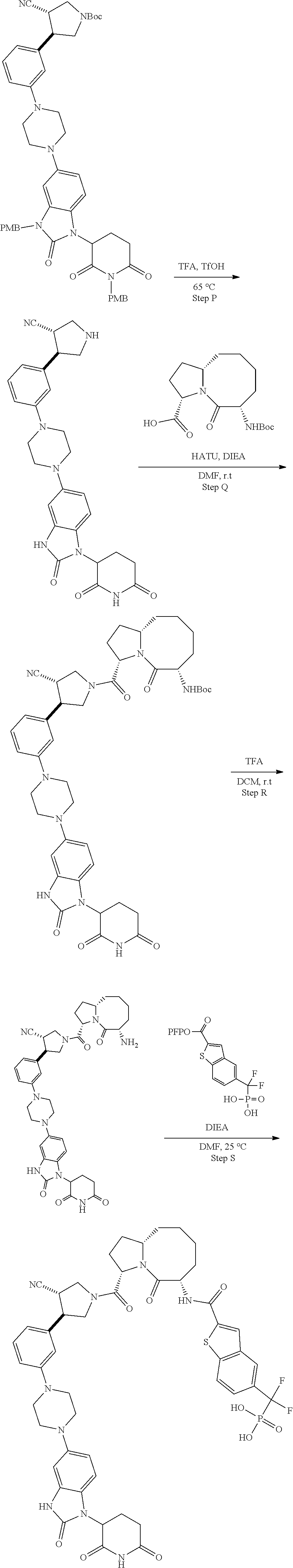

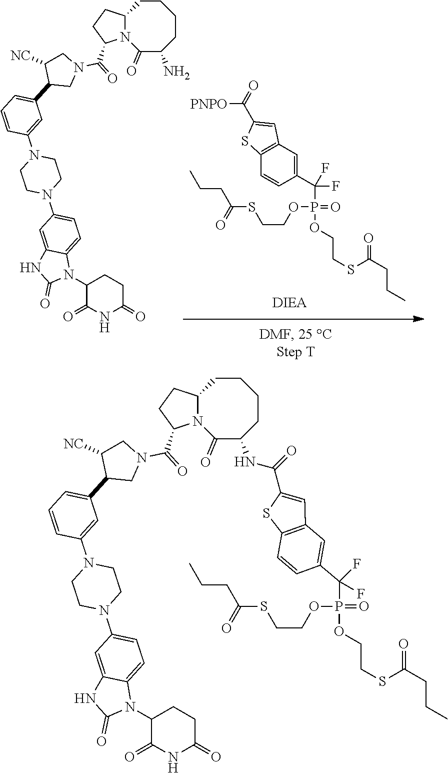

### STEP A: (E)-3-(3-bromophenyl)acrylonitrile

To a solution of diethyl (cyanomethyl)phosphonate (9.56 g, 54.0 mmol, 1 eq.) in THF (50 mL) was added (tert-butoxy)potassium (6.05 g, 54.0 mmol, 1 eq.) at 0° C. The solution was stirred at room temperature for 1 hr, then 3-bromobenzaldehyde (10 g, 54.0 mmol, 1 eq.) was added to the solution, the solution was stirred at room temperature for additional 1 hr. After completion, H₂O (100 mL) was added to the solution, the solution was extracted with EtOAc (200 mL×3). The organic layers were combined and washed with brine (100 mL), dried over anhydrous Na₂SO₄, and concentrated under reduced pressure. The residue was purified by flash column chromatography on silica gel to afford (2E)-3-(3-bromophenyl)prop-2-enenitrile (10.0 g, 48.0 mmol, 89%) as a yellow solid. LCMS (ESI): m/z=208 [M+H]⁺.

### STEP B: rac-(3S,4R)-1-benzyl-4-(3-bromophenyl)pyrrolidine-3-carbonitrile

To a solution of (2E)-3-(3-bromophenyl)prop-2-enenitrile (10 g, 48.0 mmol, 1 eq.) in DCM (100 mL) were added benzyl(methoxymethyl)[(trimethylsilyl)methyl]amine (11.3 g, 48.0 mmol, 1 eq.) and TFA (2 mL). The solution was stirred at room temperature overnight. After completion, the reaction mixture was concentrated under reduced pressure. The residue was purified by C18 column to afford rac-1-benzyl-4-(3-bromophenyl)pyrrolidine-3-carbonitrile (4.0 g, 11.7 mmol, 25%) as a yellow oil. LCMS (ESI): m/z=341 [M+H]⁺.

### STEP C: rac-(3S,4R)-4-(3-bromophenyl)pyrrolidine-3-carbonitrile

To a solution of rac-1-benzyl-4-(3-bromophenyl)pyrrolidine-3-carbonitrile (3 g, 8.79 mmol, 1 eq.) in DCE (30 mL) was added ACECl (6.27 g, 43.9 mmol, 5 eq.). The solution was stirred at 90° C. overnight. After completion, the reaction mixture concentrated under reduced pressure. The residue was dissolved in MeOH (100 mL) and stirred under reflux for 3 hrs. The mixture was concentrated under reduced pressure to afford crude rac-4-(3-bromophenyl)pyrrolidine-3-carbonitrile (2.0 g, 7.96 mmol, 91%) as a yellow oil. LCMS (ESI): m/z=251 [M+H]⁺.

### STEP D: rac-tert-butyl (3R,4S)-3-(3-bromophenyl)-4-cyanopyrrolidine-1-carboxylate

To a solution of rac-4-(3-bromophenyl)pyrrolidine-3-carbonitrile (1.5 g, 5.97 mmol, 1 eq.) in DCM (30 mL) were added Boc₂O (2.59 g, 11.9 mmol, 2 eq.) and TEA (1.80 g, 17.9 mmol, 3 eq.) The solution was stirred at 25° C. overnight. After completion, the reaction mixture concentrated under reduced pressure. The residue was purified by flash column chromatography on silica gel to afford crude rac-tert-butyl 3-(3-bromophenyl)-4-cyanopyrrolidine-1-carboxylate (1.5 g, 4.27 mmol, 72%) as a yellow oil. LCMS (ESI): m/z=351 [M+H]⁺.

### STEP E: 5-oxotetrahydrofuran-2-carboxylic acid

To a solution of glutamic acid (21 g, 143 mmol, 1.0 eq.) in H₂O (80 mL) and HCl (12 M, 21 mL) was added a solution of NaNO₂ (14.7 g, 2.13 mol, 15.0) in H₂O (40 mL) at −5° C. And the resulting mixture was stirred for 12 hrs. After completion, the mixture was concentrated under reduced pressure and then dissolved in EtOAc (50 mL). The suspension was filtered and the filter cake was washed with EtOAc (10 mL×3). The combined filtrate was dried over anhydrous Na₂SO₄, then concentrated under reduced pressure to afford crude 5-oxotetrahydrofuran-2-carboxylic acid (10 g, quant.) as a colorless oil, which was used directly in next step without further purification.

### STEP F: N-(4-methoxybenzyl)-5-oxotetrahydrofuran-2-carboxamide

To a solution of 5-oxotetrahydrofuran-2-carboxylic acid (10 g, 76.8 mmol, 1.0 eq.) in DCM (100 mL) was added SOCl₂ (45.5 g, 383 mmol, 5.0 eq.). The solution was stirred at room temperature for 2 hrs, then concentrated under vacuum. The residue was dissolved in DCM (100 mL), then 1-(4-methoxyphenyl)methanamine (10.5 g, 76.8 mmol, 1.0 eq.) and TEA (15.4 g, 153 mmol, 2.0 eq) were added to the solution. The solution was stirred at room temperature overnight. After completion, the mixture was concentrated under reduced pressure. The residue was purified by flash column chromatography on silica gel to afford N-[(4-methoxyphenyl)methyl]-5-oxooxolane-2-carboxamide (10.0 g, 40.1 mmol, 52%) as a yellow solid. LCMS (ESI): m/z=250 [M+H]⁺.

### STEP G: 3-hydroxy-1-(4-methoxybenzyl)piperidine-2,6-dione

To a solution of N-[(4-methoxyphenyl)methyl]-5-oxooxolane-2-carboxamide (10 g, 40.1 mmol, 1 eq.) in THF (100 mL) was added (tert-butoxy)potassium (4.49 g, 40.1 mmol, 1 eq.) at −65° C. The solution was stirred at −65° C. for 4 hrs. After completion, the reaction mixture was quenched by adding H₂O (100 mL), then extracted with EtOAc (100 mL×3). The organic layers were combined and washed with brine (100 mL), dried over anhydrous Na₂SO₄, and concentrated under reduced pressure. The residue was purified by flash column chromatography on silica gel to afford 3-hydroxy-1-[(4-methoxyphenyl)methyl]piperidine-2,6-dione (5.0 g, 20.0 mmol, 50%) as a colorless oil.

LCMS (ESI): m/z=250 [M+H]⁺.

### STEP H: 1-(4-methoxybenzyl)-2,6-dioxopiperidin-3-yl trifluoromethanesulfonate

To a solution of 3-hydroxy-1-[(4-methoxyphenyl)methyl]piperidine-2,6-dione (5 g, 20.0 mmol, 1 eq.) in DCM (50 mL) were added pyridine (3.16 g, 40.0 mmol, 2 eq.) and trifluoromethanesulfonic acid anhydride (6.28 g, 20.0 mmol, 1 eq.) at 0° C. The resulting solution was stirred at room temperature overnight. After completion, the mixture was concentrated under reduced pressure. The residue was purified by flash column chromatography on silica gel to afford 1-[(4-methoxyphenyl)methyl]-2,6-dioxopiperidin-3-yl trifluoromethanesulfonate (4.0 g, 10.4 mmol, 52%) as a white solid. LCMS (ESI): m/z=382 [M+H]⁺.

### STEP I: tert-butyl 4-(3-amino-4-nitrophenyl)piperazine-1-carboxylate

To a solution of 5-fluoro-2-nitroaniline (3 g, 19.2 mmol, 1 eq.) in DMF (25 mL) were added K₂CO₃ (5.29 g, 38.4 mmol, 2 eq.) and tert-butyl piperazine-1-carboxylate (3.57 g, 19.2 mmol, 1 eq.). The solution was stirred at 80° C. overnight. After completion, the mixture was poured into H₂O (500 mL), the product was collected by filtration to afford tert-butyl 4-(3-amino-4-nitrophenyl)piperazine-1-carboxylate (5.00 g, 15.5 mmol, 81%) as a yellow solid. LCMS (ESI): m/z=323 [M+H]⁺.

### STEP J: tert-butyl 4-(3-((4-methoxybenzyl)amino)-4-nitrophenyl)piperazine-1-carboxylate

To a solution of tert-butyl 4-(3-amino-4-nitrophenyl)piperazine-1-carboxylate (3 g, 9.30 mmol, 1 eq.) in DMF (30 mL) was added NaH (60 wt %) (446 mg, 18.6 mmol, 2 eq.) at 0° C., the solution was stirred at 0° C. for 30 min, then 1-(chloromethyl)-4-methoxybenzene (1.45 g, 9.30 mmol, 1 eq.) was added to the solution. The reaction mixture was warmed up to room temperature and stirred for 1 hr. After completion, the reaction mixture was quenched by adding H₂O (100 mL), then extracted with EtOAc (100 mL×3). The organic layers were combined and washed with brine (100 mL), dried over anhydrous Na₂SO₄, and concentrated under reduced pressure. The residue was purified by flash column chromatography on silica gel to afford tert-butyl 4-(3-{[(4-methoxyphenyl)methyl]amino}-4-nitrophenyl)piperazine-1-carboxylate (3.0 g, 6.77 mmol, 73%) as a yellow solid. LCMS (ESI): m/z=443 [M+H]⁺.

### STEP K: tert-butyl 4-(4-amino-3-((4-methoxybenzyl)amino)phenyl)piperazine-1-carboxylate

To a solution of tert-butyl 4-(3-{[(4-methoxyphenyl)methyl]amino}-4-nitrophenyl)piperazine-1-carboxylate (2 g, 4.51 mmol, 1 eq.) in EtOH (30 mL) and saturated NH₄Cl (30 mL, aq.) was added zinc (2.94 g, 45.1 mmol, 10 eq.), the solution was stirred at room temperature for 3 hrs. After completion, the suspension was filtered through a pad of Celite®, the filter cake was washed with MeOH (20 mL). The combined filtrates were concentrated under reduced pressure. The residue was dissolved in H₂O (30 mL) and extracted with EtOAc (50 mL×3). The organic layers were combined and washed with brine (100 mL), dried over anhydrous Na₂SO₄, and concentrated under reduced pressure. The residue was purified by flash column chromatography on silica gel to afford tert-butyl 4-(4-amino-3-{[(4-methoxyphenyl)methyl]amino}phenyl)piperazine-1-carboxylate (1.80 g, 4.36 mmol, 97%) as a yellow solid. LCMS (ESI): m/z=413 [M+H]⁺.

### STEP L: tert-butyl 4-(3-(4-methoxybenzyl)-2-oxo-2,3-dihydro-1H-benzo[d]imidazol-5-yl)piperazine-1-carboxylate

To a solution of tert-butyl 4-(4-amino-3-{[(4-methoxyphenyl)methyl]amino}phenyl)piperazine-1-carboxylate (1.5 g, 3.63 mmol, 1 eq.) in THF (20 mL) was added CDI (1.17 g, 7.26 mmol, 2 eq.). The solution was stirred at 50° C. for 1 hr. The mixture was concentrated under reduced pressure. The residue was purified by flash column chromatography on silica gel to afford tert-butyl 4-{3-[(4-methoxyphenyl)methyl]-2-oxo-2,3-dihydro-1H-1,3-benzodiazol-5-yl}piperazine-1-carboxylate (1.50 g, 3.42 mmol, 94%) as a yellow solid. LCMS (ESI): m/z=439 [M+H]⁺.

### STEP M: tert-butyl 4-(3-(4-methoxybenzyl)-2-oxo-2,3-dihydro-1H-benzo[d]imidazol-5-yl)piperazine-1-carboxylate

To a solution of tert-butyl 4-{3-[(4-methoxyphenyl)methyl]-2-oxo-2,3-dihydro-1H-1,3-benzodiazol-5-yl}piperazine-1-carboxylate (1 g, 2.28 mmol, 1 eq.) in THF (10 mL) was added LDA (1.2 mL, 2 M in THF, 1.05 eq.) at 0° C. and the mixture was stirred at 0° C. for 1 hr, then 1-[(4-methoxyphenyl)methyl]-2,6-dioxopiperidin-3-yl trifluoromethanesulfonate (869 mg, 2.28 mmol, 1 eq.) was added to the solution. The solution was warmed up to room temperature and stirred for 1 hr. After completion, the reaction mixture was quenched by adding H₂O (20 mL), then extracted with EtOAc (50 mL×3). The organic layers were combined and washed with brine (20 mL), dried over anhydrous Na₂SO₄, and concentrated under reduced pressure. The residue was purified by flash column chromatography on silica gel to afford tert-butyl 4-{3-[(4-methoxyphenyl)methyl]-1-{1-[(4-methoxyphenyl)methyl]-2,6-dioxopiperidin-3-yl}-2-oxo-2,3-dihydro-1H-1,3-benzodiazol-5-yl}piperazine-1-carboxylate (1 g, 1.49 mmol, 66%) as a yellow solid. LCMS (ESI): m/z=670 [M+H]⁺.

### STEP N: 1-(4-methoxybenzyl)-3-(3-(4-methoxybenzyl)-2-oxo-5-(piperazin-1-yl)-2,3-dihydro-1H-benzo[d]imidazol-1-yl)piperidine-2,6-dione

A solution of tert-butyl 4-{3-[(4-methoxyphenyl)methyl]-1-{1-[(4-methoxyphenyl)methyl]-2,6-dioxopiperidin-3-yl}-2-oxo-2,3-dihydro-1H-1,3-benzodiazol-5-yl}piperazine-1-carboxylate (1 g, 1.49 mmol, 1 eq.) in HCl/Ethyl Acetate (25 mL, 3 M) was stirred at room temperature for 1 hr. After completion, the reaction mixture was concentrated under reduced pressure. The residue was dissolved in H₂O (10 mL), cooled down in an ice bath, then neutralized carefully with NaHCO₃ (aq.) until the pH was adjusted to pH=9. The resulting mixture was extracted with DCM (20 mL×3), and the combined organic layers were washed with brine (10 mL), dried over with anhydrous Na₂SO₄, then concentrated under reduced pressure to give crude 1-[(4-methoxyphenyl)methyl]-3-{3-[(4-methoxyphenyl)methyl]-2-oxo-5-(piperazin-1-yl)-2,3-dihydro-1H-1,3-benzodiazol-1-yl}piperidine-2,6-dione (800 mg, quant.) as a blue solid, which was used in next step directly without further purification. LCMS (ESI): m/z=570 [M+H]⁺.

### STEP O: tert-butyl (rel-3S,4R)-3-cyano-4-(3-(4-(3-(4-methoxybenzyl)-1-(1-(4-methoxybenzyl)-2,6-dioxopiperidin-3-yl)-2-oxo-2,3-dihydro-1H-benzo[d]imidazol-5-yl)piperazin-1-yl)phenyl)pyrrolidine-1-carboxylate

To a solution of 1-[(4-methoxyphenyl)methyl]-3-{3-[(4-methoxyphenyl)methyl]-2-oxo-5-(piperazin-1-yl)-2,3-dihydro-1H-1,3-benzodiazol-1-yl}piperidine-2,6-dione (500 mg, 877 μmol, 1 eq.) in toluene (10 mL) were added rac-tert-butyl 3-(3-bromophenyl)-4-cyanopyrrolidine-1-carboxylate (308 mg, 877 μmol, 1 eq.), Pd₂(dba)₃ (80 mg, 88 μmol, 0.1 eq.), Ruphos (82 mg, 175 μmol, 0.2 eq.) and t-BuONa (168 mg, 1.75 mmol, 2 eq.). The solution was stirred at 80° C. for 2 hrs under N₂. After completion, the solution was concentrated under reduced pressure. The residue was purified by C18 column to afford tert-butyl (rei-3S,4R)-3-cyano-4-(3-(4-(3-(4-methoxybenzyl)-1-(1-(4-methoxybenzyl)-2,6-dioxopiperidin-3-yl)-2-oxo-2,3-dihydro-1H-benzo[d]imidazol-5-yl)piperazin-1-yl)phenyl)pyrrolidine-1-carboxylate (100 mg, 119 μmol, 14%) as a white solid. LCMS (ESI): m/z=840 [M+H]⁺.

### STEP P: (rel-3S,4R)-4-(3-(4-(1-(2,6-dioxopiperidin-3-yl)-2-oxo-2,3-dihydro-1H-benzo[d]imidazol-5-yl)piperazin-1-yl)phenyl)pyrrolidine-3-carbonitrile

To a solution of tert-butyl (rel-3S,4R)-3-cyano-4-(3-(4-(3-(4-methoxybenzyl)-1-(1-(4-methoxybenzyl)-2,6-dioxopiperidin-3-yl)-2-oxo-2,3-dihydro-1H-benzo[d]imidazol-5-yl)piperazin-1-yl)phenyl)pyrrolidine-1-carboxylate (300 mg, 357 μmol, 1 eq.) in TFA (3 mL) was added trifluoromethanesulfonic acid (686 mg, 7.14 mmol, 20 eq.). The solution was stirred at 65° C. for 2 hrs. After completion, the solution was concentrated under reduced pressure. The residue was purified by C18 column to afford (rel-3S,4R)-4-(3-(4-(1-(2,6-dioxopiperidin-3-yl)-2-oxo-2,3-dihydro-1H-benzo[d]imidazol-5-yl)piperazin-1-yl)phenyl)pyrrolidine-3-carbonitrile (80 mg, 160 μmol, 45%) as a white solid. LCMS (ESI): m/z=500 [M+H]⁺.

### STEP Q: tert-butyl ((3S,6S,10aS)-3-((rel-3S,4R)-3-cyano-4-(3-(4-(1-(2,6-dioxopiperidin-3-yl)-2-oxo-2,3-dihydro-1H-benzo[d]imidazol-5-yl)piperazin-1-yl)phenyl)pyrrolidine-1-carbonyl)-5-oxodecahydropyrrolo[1,2-a]azocin-6-yl)carbamate

To a solution of (3S,6S,10aS)-6-{[(tert-butoxy)carbonyl]amino}-5-oxo-decahydropyrrolo[1,2-a]azocine-3-carboxylic acid (13 mg, 40 μmol, 1.0 eq.) in DMF (5 mL) were added HATU (15 mg, 40 μmol, 1.0 eq.), TEA (8 mg, 80 μmol, 2 eq.) and (rel-3S,4R)-4-(3-(4-(1-(2,6-dioxopiperidin-3-yl)-2-oxo-2,3-dihydro-1H-benzo[d]imidazol-5-yl)piperazin-1-yl)phenyl)pyrrolidine-3-carbonitrile (20 mg, 40 μmol, 1 eq.). The solution was stirred at room temperature for 1 hr. After completion, the mixture was purified by C18 column directly to afford tert-butyl ((3S,6S,10aS)-3-((rei-3S,4R)-3-cyano-4-(3-(4-(1-(2,6-dioxopiperidin-3-yl)-2-oxo-2,3-dihydro-1H-benzo[d]imidazol-5-yl)piperazin-1-yl)phenyl)pyrrolidine-1-carbonyl)-5-oxodecahydropyrrolo[1,2-a]azocin-6-yl)carbamate (20 mg, 25 μmol, 62%) as a white solid. LCMS (ESI): m/z=808 [M+H]⁺.

### STEP R: (rel-3S,4R)-1-((3S,6S,10aS)-6-amino-5-oxodecahydropyrrolo[1,2-a]azocine-3-carbonyl)-4-(3-(4-(1-(2,6-dioxopiperidin-3-yl)-2-oxo-2,3-dihydro-H-benzo[d]imidazol-5-yl)piperazin-1-yl)phenyl)pyrrolidine-3-carbonitrile

To a solution of tert-butyl ((3S,6S,10aS)-3-((rei-3S,4R)-3-cyano-4-(3-(4-(1-(2,6-dioxopiperidin-3-yl)-2-oxo-2,3-dihydro-1H-benzo[d]imidazol-5-yl)piperazin-1-yl)phenyl)pyrrolidine-1-carbonyl)-5-oxodecahydropyrrolo[1,2-a]azocin-6-yl)carbamate (20 g, 25 mmol, 1 eq.) in DCM (2 mL) was added TFA (1 mL). The solution was stirred at room temperature for 1 hr. After completion, the reaction mixture was concentrated under reduced pressure to afford crude (rel-3S,4R)-1-((3S,6S,10aS)-6-amino-5-oxodecahydropyrrolo[1,2-a]azocine-3-carbonyl)-4-(3-(4-(1-(2,6-dioxopiperidin-3-yl)-2-oxo-2,3-dihydro-1H-benzo[d]imidazol-5-yl)piperazin-1-yl)phenyl)pyrrolidine-3-carbonitrile (15 mg, quant.) as a yellow oil, which was used in next step directly without further purification. LCMS (ESI): m/z=708 [M+H]⁺.

### STEP S: ((2-(((3S,6S,10aS)-3-((rel-3S,4R)-3-cyano-4-(3-(4-(1-(2,6-dioxopiperidin-3-yl)-2-oxo-2,3-dihydro-1H-benzo[d]imidazol-5-yl)piperazin-1-yl)phenyl)pyrrolidine-1-carbonyl)-5-oxodecahydropyrroo[1,2-a]azocin-6-yl)carbamoyl)benzo[b]thiophen-5-yl)difluoromethyl)phosphonic acid

To a solution of (rel-3S,4R)-1-((3S,6S,10aS)-6-amino-5-oxodecahydropyrrolo[1,2-a]azocine-3-carbonyl)-4-(3-(4-(1-(2,6-dioxopiperidin-3-yl)-2-oxo-2,3-dihydro-1H-benzo[d]imidazol-5-yl)piperazin-1-yl)phenyl)pyrrolidine-3-carbonitrile (7 mg, 10 μmol, 1 eq.) in DMF (2 mL) were added TEA (5 mg, 49 μmol, 5 eq.) and [difluoro({2-[(2,3,4,5,6-pentafluorophenoxy)carbonyl]-1-benzothiophen-5-yl})methyl]phosphonic acid (5 mg, 10 μmol, 1 eq.). The resulting mixture was stirred at room temperature for 2 hrs. After completion the reaction mixture was purified by prep-HPLC to afford ((2-(((3S,6S,10aS)-3-((rel-3S,4R)-3-cyano-4-(3-(4-(1-(2,6-dioxopiperidin-3-yl)-2-oxo-2,3-dihydro-1H-benzo[d]imidazol-5-yl)piperazin-1-yl)phenyl)pyrrolidine-1-carbonyl)-5-oxodecahydropyrrolo[1,2-a]azocin-6-yl)carbamoyl)benzo[b]thiophen-5-yl)difluoromethyl)phosphonic acid (Compound 22, 1.1 mg, 1 μmol, 11%) as a white solid.

¹H NMR (400 MHz, DMSO-d6) δ 11.18-10.79 (m, 2H), 8.94-8.70 (m, 1H), 8.39-7.99 (m, 3H), 7.71-7.50 (m, 1H), 7.31-6.60 (m, 7H), 5.34-5.18 (m, 1H), 5.02-4.87 (m, 1H), 4.61-4.48 (m, 1H), 4.36-4.27 (m, 1H), 4.04-3.96 (m, 1H), 3.87-3.81 (m, 1H), 3.77-3.72 (m, 1H), 3.66-3.62 (m, 1H), 3.54-3.50 (m, 1H), 3.27-3.18 (m, 4H), 2.93-2.83 (m, 1H), 2.74-2.61 (m, 1H), 2.33-2.17 (m, 1H), 2.09-1.95 (m, 3H), 1.89-1.72 (m, 5H), 1.67-1.46 (m, 3H), 1.35-1.14 (m, 4H).

LCMS (ESI): m/z=998 [M+H]⁺.

### STEP T. S,S(((((2-(((3S,6S,10aS)-3-((rel-3S,4R)-3-cyano-4-(3-(4-(1-(2,6-dioxopiperidin-3-yl)-2-oxo-2,3-dihydro-1H-benzo[d]imidazol-5-yl)piperazin-1-yl)phenyl)pyrrolidine-1-carbonyl)-5-oxodecahydropyrrolo[1,2-a]azocin-6-yl)carbamoyl)benzo[b]thiophen-5-yl)difluoromethyl)phosphoryl)bis(oxy))bis(ethane-2,1-diyl)) dibutanethioate

To a solution of (rel-3S,4R)-1-((3S,6S,10aS)-6-amino-5-oxodecahydropyrrolo[1,2-a]azocine-3-carbonyl)-4-(3-(4-(1-(2,6-dioxopiperidin-3-yl)-2-oxo-2,3-dihydro-1H-benzo[d]imidazol-5-yl)piperazin-1-yl)phenyl)pyrrolidine-3-carbonitrile (7 mg, 10 μmol, 1 eq.) in DMF (2 mL) were added TEA (5 mg, 49 μmol, 5 eq.) and 4-nitrophenyl 5-({bis[2-(butanoylsulfanyl)ethoxy]phosphoryl}difluoromethyl)-1-benzothiophene-2-carboxylate (7 mg, 10 μmol, 1 eq.). The solution was stirred at room temperature for 1 hr. After completion the reaction mixture was purified by prep-HPLC to afford S,S [symbol] (((((2-(((3S,6S,10aS)-3-((rei-3S,4R)-3-cyano-4-(3-(4-(1-(2,6-dioxopiperidin-3-yl)-2-oxo-2,3-dihydro-1H-benzo[d]imidazol-5-yl)piperazin-1-yl)phenyl)pyrrolidine-1-carbonyl)-5-oxodecahydropyrrolo[1,2-a]azocin-6-yl)carbamoyl)benzo[b]thiophen-5-yl)difluoromethyl)phosphoryl)bis(oxy))bis(ethane-2,1-diyl)) dibutanethioate (Compound 23, 2.1 mg, 2 μmol, 17%) as a white solid.

¹H NMR (400 MHz, DMSO-d6) δ 11.13-10.95 (m, 1H), 10.80 (s, 1H), 8.99-8.82 (m, 1H), 8.40-8.06 (m, 3H), 7.63-7.51 (m, 1H), 7.29-7.17 (m, 1H), 7.12-7.03 (m, 1H), 6.98-6.79 (m, 3H), 6.71-6.60 (m, 2H), 5.36-5.16 (m, 1H), 5.04-4.89 (m, 1H), 4.62-3.42 (m, 12H), 3.20-3.07 (m, 8H), 2.95-2.81 (m, 1H), 2.74-2.55 (m, 3H), 2.31-1.45 (m, 20H), 1.31-1.18 (m, 4H), 0.91-0.80 (m, 6H).

LCMS (ESI): m/z=1258.7 [M+H]⁺.

### Preparation of ((2-(((3S,6S,10aS)-3-((6R)-6-(1-(1-(1-(2,6-dioxopiperidin-3-yl)-2-oxo-2,3-dihydro-1H-benzo[d]imidazol-5-yl)piperidin-4-yl)-2-oxo-1,2-dihydropyridin-4-yl)-4-azaspiro[2.4]heptane-4-carbonyl)-5-oxodecahydropyrrolo[1,2-a]azocin-6-yl)carbamoyl)benzo[b]thiophen-5-yl)difluoromethyl)phosphonic acid (Compound 24) and S,S((((2-(((3S,6S,10aS)-3-((6R)-6-(1-(1-(1-(2,6-dioxopiperidin-3-yl)-2-oxo-2,3-dihydro-1H-benzo[d]imidazol-5-yl)piperidin-4-yl)-2-oxo-1,2-dihydropyridin-4-yl)-4-azaspiro[2.4]heptane-4-carbonyl)-5-oxodecahydropyrrolo[1,2-a]azocin-6-yl)carbamoyl)benzo[b]thiophen-5-yl)difluoromethyl)phosphoryl)bis(oxy))bis(ethane-2,1-diyl)) dibutanethioate (Compound 25)

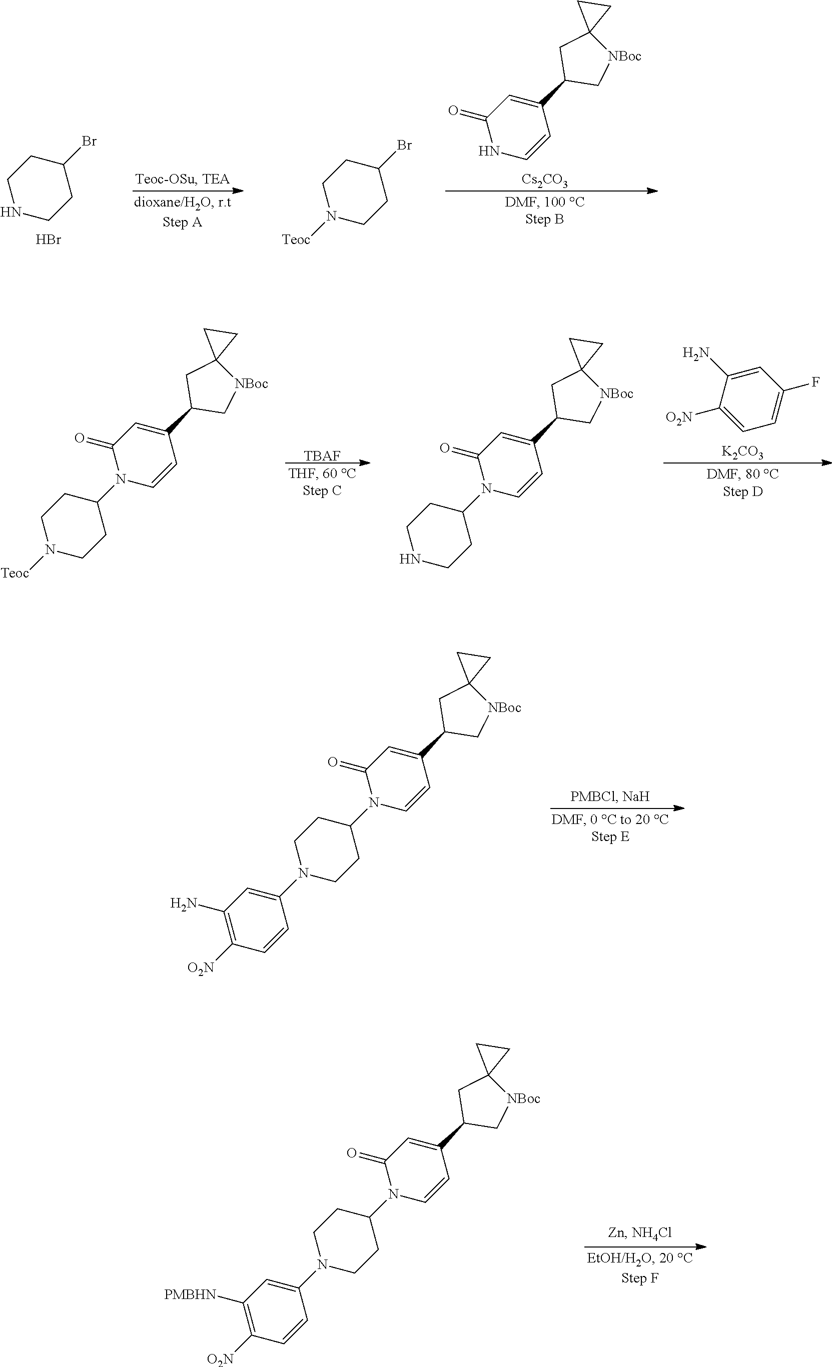

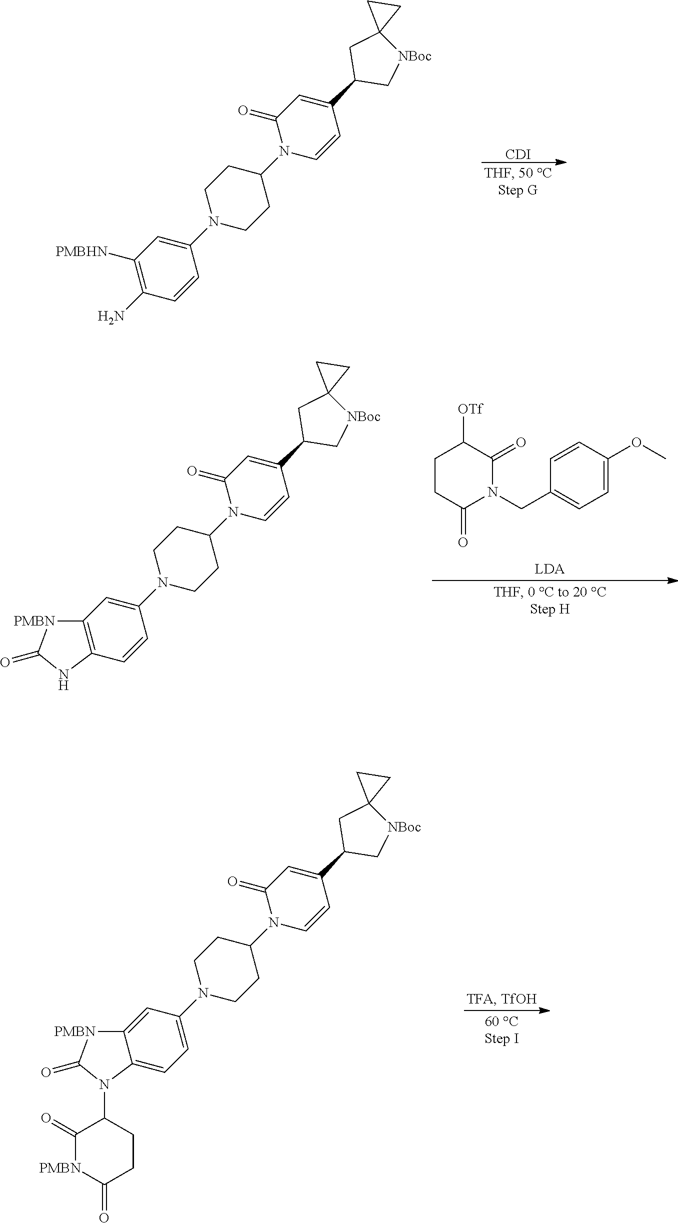

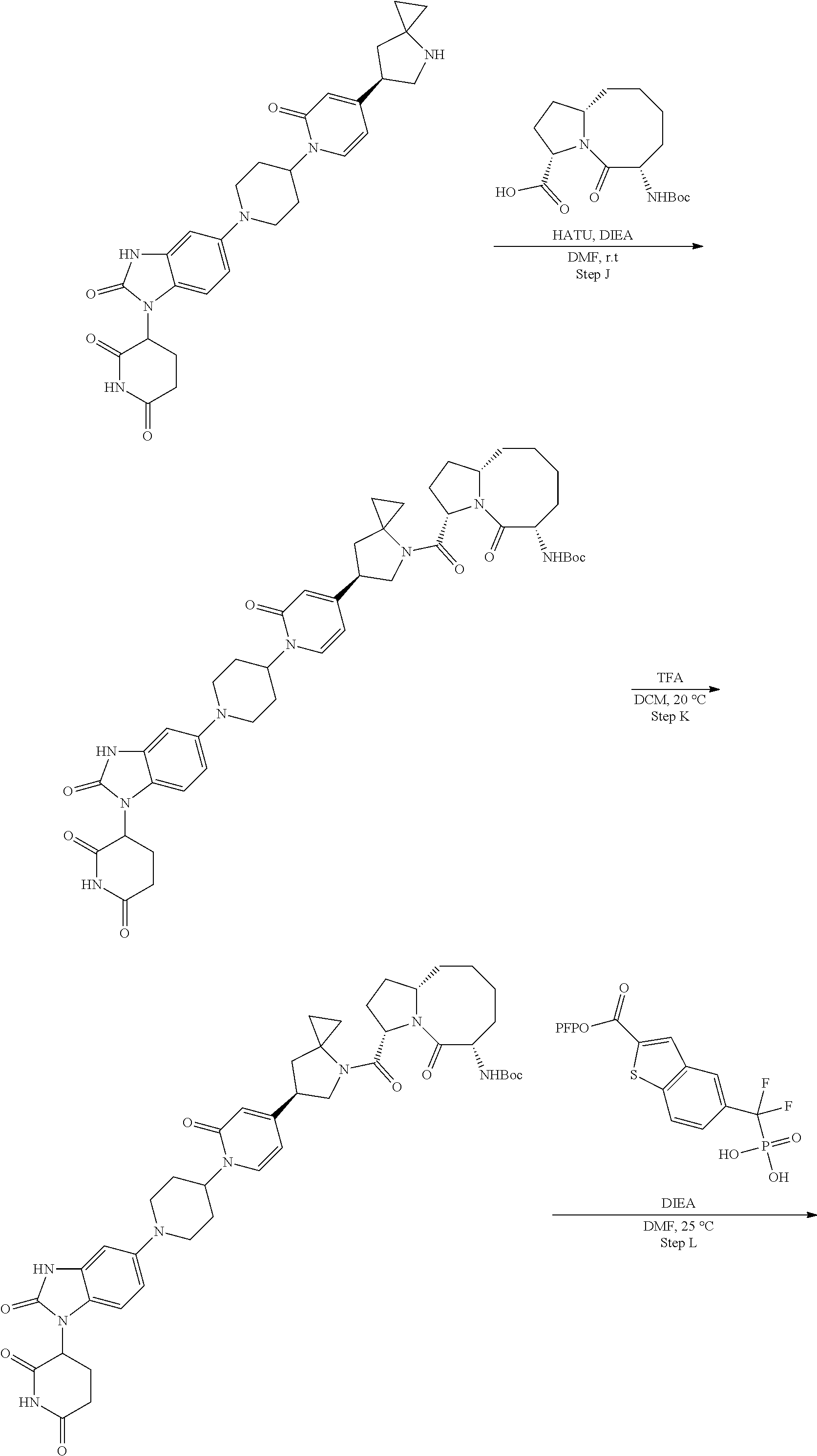

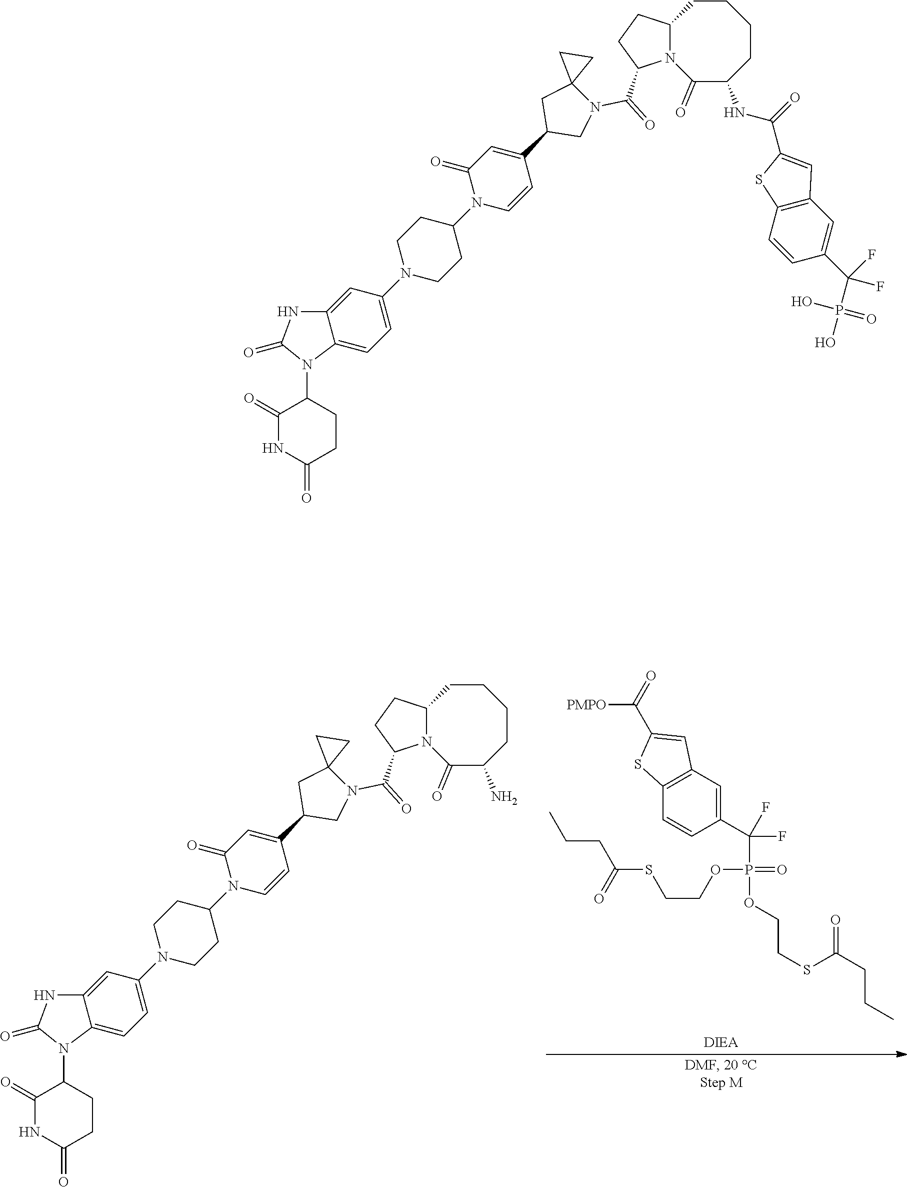

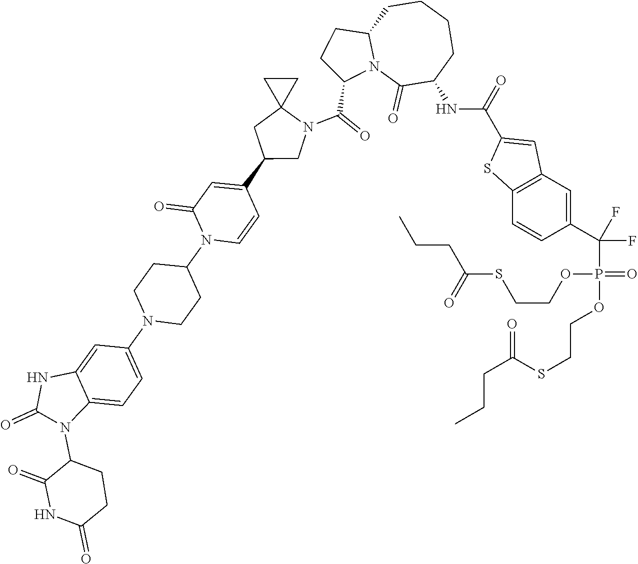 [SMILES: CCCC(=O)SCCOP(=O)(OCCSC(=O)CCC)C(F)(F)c1ccc2sc(C(=O)N[C@H]3CCCC[C@H]4CC[C@@H](C(=O)N5C[C@@H](c6ccn(C7CCN(c8ccc9c(c8)[nH]c(=O)n9C8CCC(=O)NC8=O)CC7)c(=O)c6)CC56CC6)N4C3=O)cc2c1]

### STEP A: 2-(trimethylsilyl)ethyl 4-bromopiperidine-1-carboxylate

To a solution of 4-bromopiperidine hydrobromide (9 g, 37.0 mmol, 1 eq.) and TEA (7.8 mL, 55.5 mmol, 1.5 eq.) in dioxane/H₂O (100 mL/100 mL) was added 2,5-dioxopyrrolidin-1-yl (2-(trimethylsilyl)ethyl) carbonate (12.5 g, 48.1 mmol, 1.3 eq.) at 20° C. The solution was stirred at room temperature for 12 hrs. After completion, H₂O (300 mL) was added to the solution, the solution was extracted with EtOAc (200 mL×3). The organic layers were combined and washed with brine (300 mL), dried over anhydrous Na₂SO₄, and concentrated under reduced pressure. The residue was purified by flash column chromatography on silica gel to afford 2-(trimethylsilyl)ethyl 4-bromopiperidine-1-carboxylate (10 g, 32.6 mmol, 88%) as a colorless oil. LC-MS (ESI) m/z=308 [M+H]⁺.

### STEP B: tert-butyl (R)-6-(2-oxo-1-(1-((2-(trimethylsilyl)ethoxy)carbonyl)piperidin-4-yl)-1,2-dihydropyridin-4-yl)-4-azaspiro[2.4]heptane-4-carboxylate

A solution of tert-butyl (R)-6-(2-oxo-1,2-dihydropyridin-4-yl)-4-azaspiro[2.4]heptane-4-carboxylate (1 g, 3.45 mmol, 1.0 eq.), 2-(trimethylsilyl)ethyl 4-bromopiperidine-1-carboxylate (5.2 g, 17.3 mmol, 5 eq.) and Cs₂CO₃ (2.2 g, 6.9 mmol, 2eq.) in DMF (10 mL) was stirred at 100° C. overnight. After completion, H₂O (300 mL) was added to the solution, the solution was extracted with EtOAc (200 mL×3). The organic layers were combined and washed with brine (300 mL), dried over anhydrous Na₂SO₄, and concentrated under reduced pressure. The residue was purified by flash column chromatography on silica gel to afford tert-butyl (R)-6-(2-oxo-1-(1-((2-(trimethylsilyl)ethoxy)carbonyl)piperidin-4-yl)-1,2-dihydropyridin-4-yl)-4-azaspiro[2.4]heptane-4-carboxylate (600 mg, 34%) as a yellow oil. LC-MS (ESI) m/z=518 [M+H]⁺.

### STEP C: tert-butyl (R)-6-(2-oxo-1-(piperidin-4-yl)-1,2-dihydropyridin-4-yl)-4-azaspiro[2.4]heptane-4-carboxylate

A solution of tert-butyl (R)-6-(2-oxo-1-(1-((2-(trimethylsilyl)ethoxy)carbonyl)piperidin-4-yl)-1,2-dihydropyridin-4-yl)-4-azaspiro[2.4]heptane-4-carboxylate (600 mg, 1.2 mmol, 1.0 eq.) and TBAF (909.1 mg, 3.6 mmol, 3 eq.) in THF (10 mL) was stirred at 60° C. for 12 hrs. After completion, the reaction mixture concentrated under reduced pressure to afford tert-butyl (R)-6-(2-oxo-1-(piperidin-4-yl)-1,2-dihydropyridin-4-yl)-4-azaspiro[2.4]heptane-4-carboxylate (270 mg, crude) as a yellow oil, which was used in the next step without further purification. LCMS (ESI): m/z=374 [M+H]⁺.

### STEP D: tert-butyl (R)-6-(1-(1-(3-amino-4-nitrophenyl)piperidin-4-yl)-2-oxo-1,2-dihydropyridin-4-yl)-4-azaspiro[2.4]heptane-4-carboxylate

A solution of tert-butyl (R)-6-(2-oxo-1-(piperidin-4-yl)-1,2-dihydropyridin-4-yl)-4-azaspiro[2.4]heptane-4-carboxylate (270 mg, 0.72 mmol, 1.0 eq.), 5-fluoro-2-nitroaniline (113 mg, 0.72 mmol, 1.0 eq.) and K₂CO₃ (299 mg, 2.16 mmol, 3 eq.) in DMF (3 mL) was stirred at 80° C. for 16 hrs. After completion, H₂O (50 mL) was added to the solution, the solution was extracted with EtOAc (50 mL×3). The organic layers were combined and washed with brine (50 mL), dried over anhydrous Na₂SO₄, and concentrated under reduced pressure. The residue was purified by flash column chromatography on silica gel to afford tert-butyl (R)-6-(1-(1-(3-amino-4-nitrophenyl)piperidin-4-yl)-2-oxo-1,2-dihydropyridin-4-yl)-4-azaspiro[2.4]heptane-4-carboxylate (280 mg, 76%) as a yellow solid. LCMS (ESI): m/z=510 [M+H]⁺.

### STEP E: tert-butyl (R)-6-(1-(1-(3-amino-4-nitrophenyl)piperidin-4-yl)-2-oxo-1,2-dihydropyridin-4-yl)-4-azaspiro[2.4]heptane-4-carboxylate

To a solution of tert-butyl (R)-6-(1-(1-(3-amino-4-nitrophenyl)piperidin-4-yl)-2-oxo-1,2-dihydropyridin-4-yl)-4-azaspiro[2.4]heptane-4-carboxylate (280 mg, 0.55 mmol, 1.0 eq.) in DMF was added 60% NaH (44 mg, 1.1 mmol, 2 eq.) at 0° C. To the reaction mixture was added PMBCl (94.4 mg, 0.6 mmol, 1.1 eq.). The reaction mixture was stirred at 20° C. for 0.5 hrs. After completion, H₂O (50 mL) was added to the solution, the solution was extracted with EtOAc (50 mL×3). The organic layers were combined and washed with brine (50 mL), dried over anhydrous Na₂SO₄, and concentrated under reduced pressure. The residue was purified by flash column chromatography on silica gel to afford tert-butyl (R)-6-(1-(1-(3-amino-4-nitrophenyl)piperidin-4-yl)-2-oxo-1,2-dihydropyridin-4-yl)-4-azaspiro[2.4]heptane-4-carboxylate (280 mg, 81%) as a yellow oil. LCMS (ESI): m/z=630 [M+H]⁺.

### STEP F: tert-butyl (R)-6-(1-(1-(4-amino-3-((4-methoxybenzyl)amino)phenyl)piperidin-4-yl)-2-oxo-1,2-dihydropyridin-4-yl)-4-azaspiro[2.4]heptane-4-carboxylate

A solution of tert-butyl (R)-6-(1-(1-(3-amino-4-nitrophenyl)piperidin-4-yl)-2-oxo-1,2-dihydropyridin-4-yl)-4-azaspiro[2.4]heptane-4-carboxylate (280 mg, 0.44 mmol, 1.0 eq.) and Zn (284 mg, 4.4 mmol, 10 eq) in EtOH/sat.NH₄Cl (5 mL/2 mL) was stirred at 20° C. for 0.5 hrs. After completion, the reaction mixture was concentrated under reduced pressure. The residue was purified by C18 column chromatography to afford tert-butyl (R)-6-(1-(1-(4-amino-3-((4-methoxybenzyl)amino)phenyl)piperidin-4-yl)-2-oxo-1,2-dihydropyridin-4-yl)-4-azaspiro[2.4]heptane-4-carboxylate (180 mg, 0.3 mmol, 68%) as a yellow oil. LCMS (ESI): m/z=600 [M+H]⁺.

### STEP G: tert-butyl (R)-6-(1-(1-(3-(4-methoxybenzyl)-2-oxo-2,3-dihydro-1H-benzo[d]imidazol-5-yl)piperidin-4-yl)-2-oxo-1,2-dihydropyridin-4-yl)-4-azaspiro[2.4]heptane-4-carboxylate

A solution of tert-butyl (R)-6-(1-(1-(4-amino-3-((4-methoxybenzyl)amino)phenyl)piperidin-4-yl)-2-oxo-1,2-dihydropyridin-4-yl)-4-azaspiro[2.4]heptane-4-carboxylate (180 mg, 0.3 mmol, 1.0 eq.) and CDI (100 mg, 0.6 mmol, 2 eq.) in THF (5 mL) was stirred at 50° C. for 2 hrs. After completion, the reaction mixture was diluted with H₂O (30 mL), then extracted with EtOAc (25 mL×3). The organic layers were combined and washed with brine (20 mL), dried over anhydrous Na₂SO₄, and concentrated under reduced pressure. The residue was purified by flash column chromatography on silica gel to afford tert-butyl (R)-6-(1-(1-(3-(4-methoxybenzyl)-2-oxo-2,3-dihydro-1H-benzo[d]imidazol-5-yl)piperidin-4-yl)-2-oxo-1,2-dihydropyridin-4-yl)-4-azaspiro[2.4]heptane-4-carboxylate (140 mg, 0.22 mmol, 74%) as a yellow oil. LCMS (ESI): m/z=626 [M+H]⁺.

### STEP H: tert-butyl (6R)-6-(1-(1-(3-(4-methoxybenzyl)-1-(1-(4-methoxybenzyl)-2,6-dioxopiperidin-3-yl)-2-oxo-2,3-dihydro-1H-benzo[d]imidazol-5-yl)piperidin-4-yl)-2-oxo-1,2-dihydropyridin-4-yl)-4-azaspiro[2.4]heptane-4-carboxylate

To a solution of tert-butyl (R)-6-(1-(1-(3-(4-methoxybenzyl)-2-oxo-2,3-dihydro-1H-benzo[d]imidazol-5-yl)piperidin-4-yl)-2-oxo-1,2-dihydropyridin-4-yl)-4-azaspiro[2.4]heptane-4-carboxylate (140 mg, 0.22 mmol, 1.0 eq.) in THF (5 mL) was added LDA (0.11 mL, 0.22 mmol, 1.0 eq.) at 0° C. under N₂. The reaction mixture was stirred at 0° C. for 0.5 hrs. To the reaction mixture was added 1-(4-methoxybenzyl)-2,6-dioxopiperidin-3-yl trifluoromethanesulfonate (172 mg, 0.44 mmol, 2 eq.), The reaction was warmed up to 20° C. and stirred for 1 hr. After completion, the reaction mixture was diluted with H₂O (30 mL), extracted with EtOAc (20 mL×3). The organic layers were combined and washed with brine (50 mL), dried over anhydrous Na₂SO₄, and concentrated under reduced pressure. The residue was purified by flash column chromatography on silica gel to afford tert-butyl (6R)-6-(1-(1-(3-(4-methoxybenzyl)-1-(1-(4-methoxybenzyl)-2,6-dioxopiperidin-3-yl)-2-oxo-2,3-dihydro-1H-benzo[d]imidazol-5-yl)piperidin-4-yl)-2-oxo-1,2-dihydropyridin-4-yl)-4-azaspiro[2.4]heptane-4-carboxylate (170 mg, 0.20 mmol, 90%) as a yellow solid. LCMS (ESI): m/z=857 [M+H]⁺.

### STEP I: 3-(2-oxo-5-(4-(2-oxo-4-((R)-4-azaspiro[2.4]heptan-6-yl)pyridin-1(2H)-yl)piperidin-1-yl)-2,3-dihydro-1H-benzo[d]imidazol-1-yl)piperidine-2,6-dione

A solution of tert-butyl (6R)-6-(1-(1-(3-(4-methoxybenzyl)-1-(1-(4-methoxybenzyl)-2,6-dioxopiperidin-3-yl)-2-oxo-2,3-dihydro-1H-benzo[d]imidazol-5-yl)piperidin-4-yl)-2-oxo-1,2-dihydropyridin-4-yl)-4-azaspiro[2.4]heptane-4-carboxylate (170 mg, 0.20 mmol, 1.0 eq.) and TfOH (60 mg, 0.40 mmol, 2.0 eq.) in TFA (2 mL) was heated at 60° C. and stirred for 12 hrs. After completion, the reaction mixture was concentrated under reduced pressure. The residue was purified by C18 column to afford 3-(2-oxo-5-(4-(2-oxo-4-((R)-4-azaspiro[2.4]heptan-6-yl)pyridin-1(2H)-yl)piperidin-1-yl)-2,3-dihydro-1H-benzo[d]imidazol-1-yl)piperidine-2,6-dione (100 mg, 0.13 mmol, 66%) as a white solid.

LCMS (ESI): m/z=517 [M+H]⁺.

### STEP J: tert-butyl ((3S,6S,10aS)-3-((6R)-6-(1-(1-(1-(2,6-dioxopiperidin-3-yl)-2-oxo-2,3-dihydro-1H-benzo[d]imidazol-5-yl)piperidin-4-yl)-2-oxo-1,2-dihydropyridin-4-yl)-4-azaspiro[2.4]heptane-4-carbonyl)-5-oxodecahydropyrrolo[1,2-a]azocin-6-yl)carbamate

To a solution of (3S,6S,10aS)-6-((tert-butoxycarbonyl)amino)-5-oxodecahydropyrrolo[1,2-a]azocine-3-carboxylic acid (22 mg, 66 μmol, 1 eq.) and HATU (30 mg, 79 μmol, 1.2 eq.) in DMF (2 mL) were added 3-(2-oxo-5-(4-(2-oxo-4-((R)-4-azaspiro[2.4]heptan-6-yl)pyridin-1(2H)-yl)piperidin-1-yl)-2,3-dihydro-1H-benzo[d]imidazol-1-yl)piperidine-2,6-dione (50 mg, 66 μmol, 1 eq.) and DIEA (0.66 mL, 5 eq). The resulting mixture was stirred at room temperature for 1 hr. After completion, the reaction mixture was purified by C18 column to afford tert-butyl ((3S,6S,10aS)-3-((6R)-6-(1-(1-(1-(2,6-dioxopiperidin-3-yl)-2-oxo-2,3-dihydro-1H-benzo[d]imidazol-5-yl)piperidin-4-yl)-2-oxo-1,2-dihydropyridin-4-yl)-4-azaspiro[2.4]heptane-4-carbonyl)-5-oxodecahydropyrrolo[1,2-a]azocin-6-yl)carbamate (28 mg, 34 μmol, 51%) as a white solid. LCMS (ESI): m/z=825 (M+H)+.

### STEP K: 3-(5-(4-(4-((R)-4-((3S,6S,10aS)-6-amino-5-oxodecahydropyrrolo[1,2-a]azocine-3-carbonyl)-4-azaspiro[2.4]heptan-6-yl)-2-oxopyridin-1(2H)-yl)piperidin-1-yl)-2-oxo-2,3-dihydro-1H-benzo[d]imidazol-1-yl)piperidine-2,6-dione

A solution of tert-butyl ((3S,6S,10aS)-3-((6R)-6-(1-(1-(1-(2,6-dioxopiperidin-3-yl)-2-oxo-2,3-dihydro-1H-benzo[d]imidazol-5-yl)piperidin-4-yl)-2-oxo-1,2-dihydropyridin-4-yl)-4-azaspiro[2.4]heptane-4-carbonyl)-5-oxodecahydropyrrolo[1,2-a]azocin-6-yl)carbamate (28 mg, 34 μmol, 1.0 eq.) in DCM/TFA (3 mL/1 mL) was stirred at 20° C. for 1 hr. After completion, the reaction mixture was concentrated under reduced pressure to give 3-(5-(4-(4-((R)-4-((3S,6S,10aS)-6-amino-5-oxodecahydropyrrolo[1,2-a]azocine-3-carbonyl)-4-azaspiro[2.4]heptan-6-yl)-2-oxopyridin-1(2H)-yl)piperidin-1-yl)-2-oxo-2,3-dihydro-1H-benzo[d]imidazol-1-yl)piperidine-2,6-dione (24 mg, crude) as a yellow solid, which was used in the next step without further purification. LCMS (ESI): m/z=725 (M+H)+.

### STEP L: ((2-(((3S,6S,10aS)-3-((6R)-6-(1-(1-(1-(2,6-dioxopiperidin-3-yl)-2-oxo-2,3-dihydro-1H-benzo[d]imidazol-5-yl)piperidin-4-yl)-2-oxo-1,2-dihydropyridin-4-yl)-4-azaspiro[2.4]heptane-4-carbonyl)-5-oxodecahydropyrrolo[1,2-a]azocin-6-yl)carbamoyl)benzo[b]thiophen-5-yl)difluoromethyl)phosphonic acid

A solution of 3-(5-(4-(4-((R)-4-((3S,6S,10aS)-6-amino-5-oxodecahydropyrrolo[1,2-a]azocine-3-carbonyl)-4-azaspiro[2.4]heptan-6-yl)-2-oxopyridin-1(2H)-yl)piperidin-1-yl)-2-oxo-2,3-dihydro-1H-benzo[d]imidazol-1-yl)piperidine-2,6-dione (12 mg, 17 μmol, 1.0 eq.), DIEA (13 mg, 99 μmol, 6 eq.) and (difluoro(2-((perfluorophenoxy)carbonyl)benzo[b]thiophen-5-yl)methyl)phosphonic acid (8 mg, 17 μmol, 1.0 eq.) in DMF (1 mL) was stirred at 25° C. for 0.5 hrs. After completion, the reaction mixture was purified by prep-HPLC to afford ((2-(((3S,6S,10aS)-3-((6R)-6-(1-(1-(1-(2,6-dioxopiperidin-3-yl)-2-oxo-2,3-dihydro-1H-benzo[d]imidazol-5-yl)piperidin-4-yl)-2-oxo-1,2-dihydropyridin-4-yl)-4-azaspiro[2.4]heptane-4-carbonyl)-5-oxodecahydropyrrolo[1,2-a]azocin-6-yl)carbamoyl)benzo[b]thiophen-5-yl)difluoromethyl)phosphonic acid (Compound 24, 3 mg, 3 μmol, 18%) as a white solid.

¹H NMR (400 MHz, DMSO-d6) δ 11.31-10.93 (m, 2H), 9.02-8.73 (m, 1H), 8.31 (s, 1H), 8.17-7.96 (m, 3H), 7.72-7.45 (m, 1H), 7.18-6.89 (m, 4H), 6.80 (s, 1H), 5.43-5.11 (m, 2H), 5.05-4.83 (m, 1H), 4.62-4.41 (m, 1H), 4.31-4.10 (m, 2H), 3.81-3.74 (m, 2H), 2.96-2.82 (m, 2H), 2.77-2.55 (m, 4H), 2.36-2.14 (m, 4H), 2.10-1.70 (m, 13H), 1.63-1.38 (m, 4H), 1.28-1.14 (m, 1H), 0.60-0.36 (m, 2H).

LCMS (ESI): m/z=1015 [M+H]⁺.

### STEP M: S,S((((2-(((3S,6S,10aS)-3-((6R)-6-(1-(1-(1-(2,6-dioxopiperidin-3-yl)-2-oxo-2,3-dihydro-1H-benzo[d]imidazol-5-yl)piperidin-4-yl)-2-oxo-1,2-dihydropyridin-4-yl)-4-azaspiro[2.4]heptane-4-carbonyl)-5-oxodecahydropyrrolo[1,2-a]azocin-6-yl)carbamoyl)benzo[b]thiophen-5-yl)difluoromethyl)phosphoryl)bis(oxy))bis(ethane-2,1-diyl)) dibutanethioate

A solution of 3-(5-(4-(4-((R)-4-((3S,6S,10aS)-6-amino-5-oxodecahydropyrrolo[1,2-a]azocine-3-carbonyl)-4-azaspiro[2.4]heptan-6-yl)-2-oxopyridin-1(2H)-yl)piperidin-1-yl)-2-oxo-2,3-dihydro-1H-benzo[d]imidazol-1-yl)piperidine-2,6-dione (12 mg, 17 μmol, 1.0 eq.), DIEA (13 mg, 100 μmol, 6 eq.) and 4-nitrophenyl 5-((bis(2-(butyrylthio)ethoxy)phosphoryl)difluoromethyl)benzo[b]thiophene-2-carboxylate (11 mg, 17 μmol, 1 eq.) was stirred at 20° C. for 2 hrs. After completion, the reaction mixture was [symbol] purified by prep-HPLC to afford S,S [symbol] ((((2-(((3S,6S,10aS)-3-((6R)-6-(1-(1-(1-(2,6-dioxopiperidin-3-yl)-2-oxo-2,3-dihydro-1H-benzo[d]imidazol-5-yl)piperidin-4-yl)-2-oxo-1,2-dihydropyridin-4-yl)-4-azaspiro[2.4]heptane-4-carbonyl)-5-oxodecahydropyrrolo[1,2-a]azocin-6-yl)carbamoyl)benzo[b]thiophen-5-yl)difluoromethyl)phosphoryl)bis(oxy))bis(ethane-2,1-diyl)) dibutanethioate (Compound 25, 1.8 mg, 1 μmol, 9%) as a white solid.

¹H NMR (400 MHz, DMSO-d6) δ 11.04 (s, 1H), 10.78 (s, 1H), 9.06-8.72 (m, 1H), 8.41-8.28 (m, 1H), 8.22-7.99 (m, 3H), 7.65-7.46 (m, 1H), 7.05-6.47 (m, 5H), 5.38-4.83 (m, 3H), 4.60-4.46 (m, 1H), 4.32-4.05 (m, 6H), 3.83-3.70 (m, 1H), 3.57-3.47 (m, 1H), 3.18-3.08 (m, 4H), 2.97-2.81 (m, 3H), 2.76-2.58 (m, 3H), 2.36-1.42 (m, 26H), 1.31-1.14 (m, 4H), 0.94-0.74 (m, 6H), 0.59-0.37 (m, 2H).

LCMS (ESI): m/z=1275 [M+H]⁺.

### Preparation of S,S((((2-(((3S,6S,10aS)-3-((6R)-6-(1-(2-(2-(2-((2-(2,6-dioxopiperidin-3-yl)-1-oxoisoindolin-4-yl)oxy)ethoxy)ethoxy)ethyl)-2-oxo-1,2-dihydropyridin-4-yl)-4-azaspiro[2.4]heptane-4-carbonyl)-5-oxodecahydropyrrolo[1,2-a]azocin-6-yl)carbamoyl)benzo[b]thiophen-5-yl)difluoromethyl)phosphoryl)bis(oxy))bis(ethane-2,1-diyl)) dibutanethioate (Compound 26) and ((2-(((3S,6S,10aS)-3-((6R)-6-(1-(2-(2-(2-((2-(2,6-dioxopiperidin-3-yl)-1-oxoisoindolin-4-yl)oxy)ethoxy)ethoxy)ethyl)-2-oxo-1,2-dihydropyridin-4-yl)-4-azaspiro[2.4]heptane-4-carbonyl)-5-oxodecahydropyrrolo[1,2-a]azocin-6-yl)carbamoyl)benzo[b]thiophen-5-yl)difluoromethyl)phosphonic acid (Compound 27)

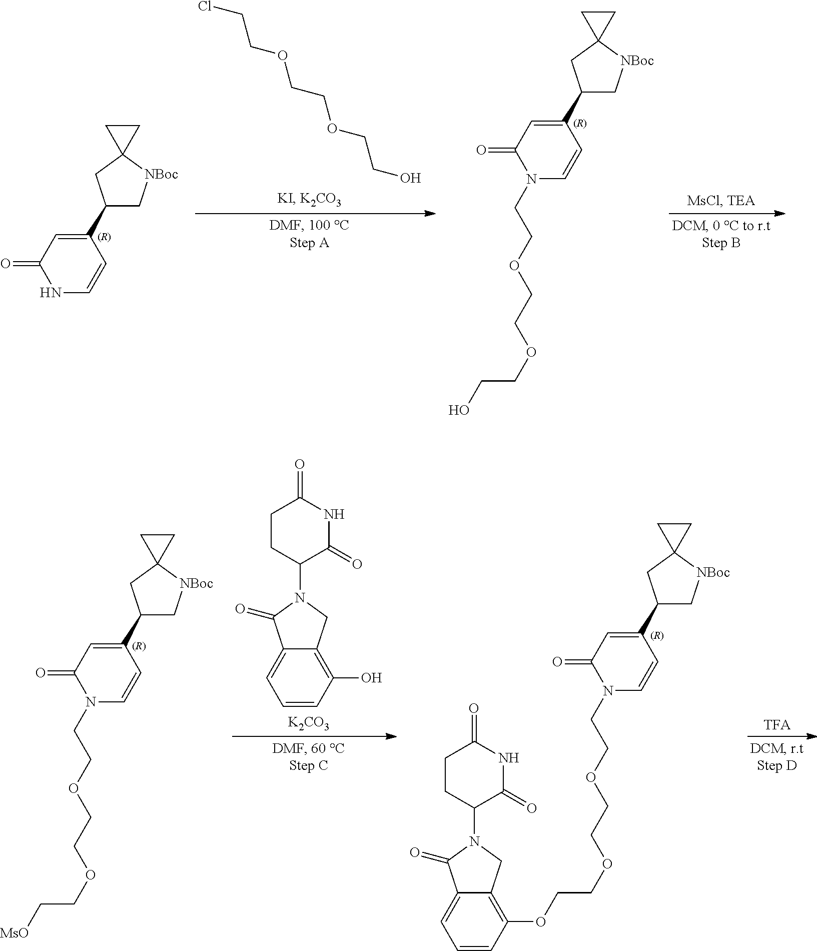

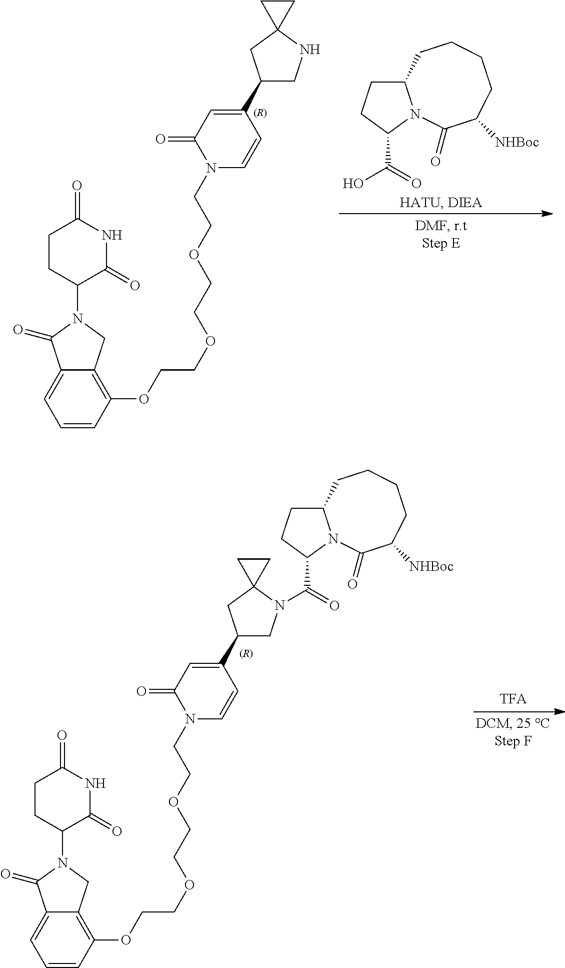

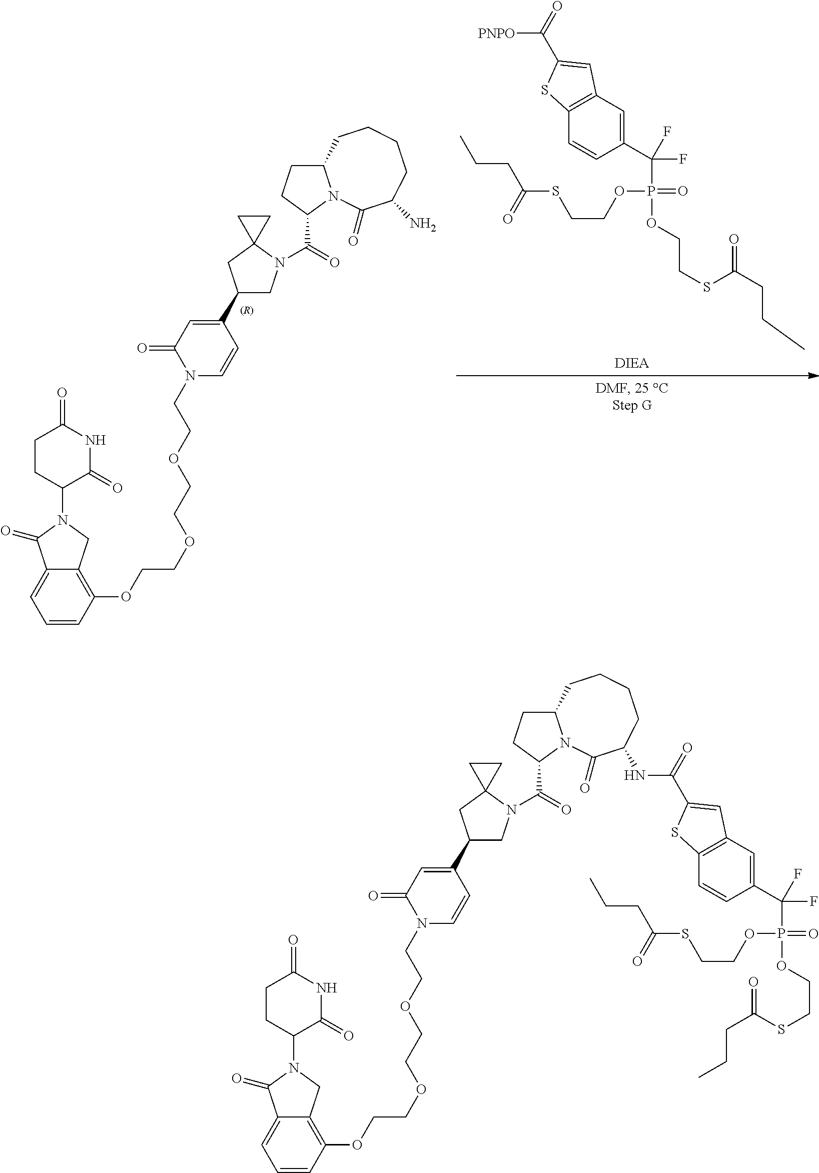

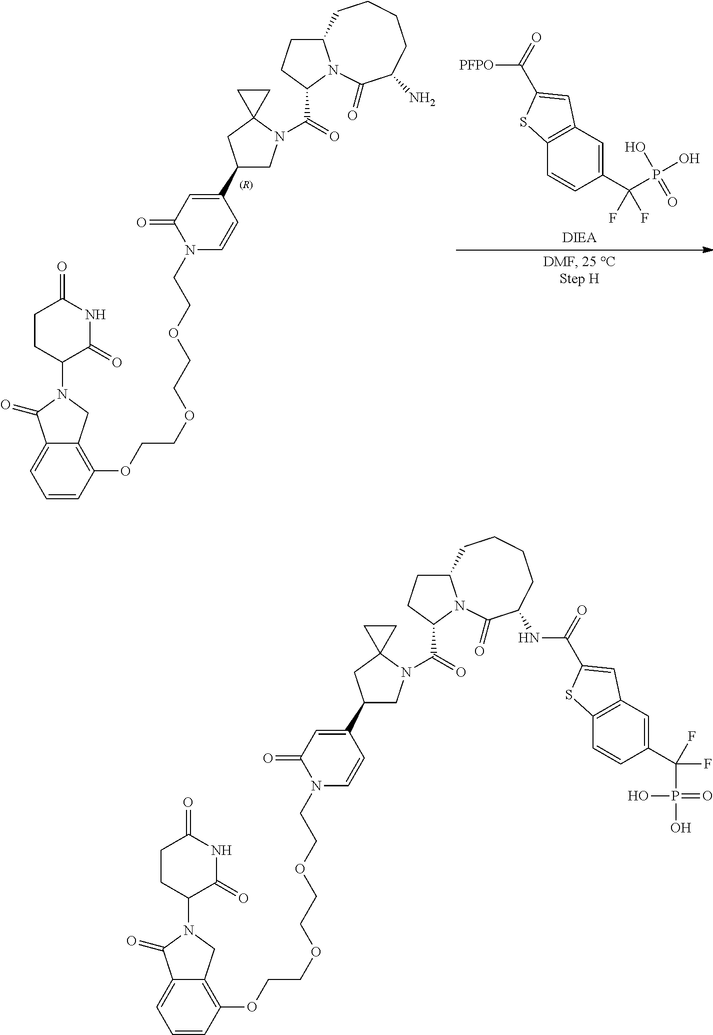

### STEP A: tert-butyl (R)-6-(1-(2-(2-(2-hydroxyethoxy)ethoxy)ethyl)-2-oxo-1,2-dihydropyridin-4-yl)-4-azaspiro[2.4]heptane-4-carboxylate

To a solution of tert-butyl (R)-6-(2-oxo-1,2-dihydropyridin-4-yl)-4-azaspiro[2.4]heptane-4-carboxylate (700 mg, 2.4 mmol, 1.0 eq.) in DMF (10 mL) were added 2-(2-(2-chloroethoxy)ethoxy)ethan-1-ol (490 mg, 2.9 mmol, 1.2 eq.), KI (200 mg, 1.2 mmol, 0.5 eq.) and K₂CO₃ (670 mg, 4.8 mmol, 2.0 eq.). The reaction mixture was stirred at 100° C. for 2 hrs. After completion, the reaction mixture was purified directly by Biotage® C18 column chromatography to afford tert-butyl tert-butyl (R)-6-(1-(2-(2-(2-hydroxyethoxy)ethoxy)ethyl)-2-oxo-1,2-dihydropyridin-4-yl)-4-azaspiro[2.4]heptane-4-carboxylate (400 mg, 0.95 mmol, 39%) as a yellow solid. LCMS (ESI): m/z=423 [M+H]⁺.

### STEP B: tert-butyl (R)-6-(1-(2-(2-(2-((methylsulfonyl)oxy)ethoxy)ethoxy)ethyl)-2-oxo-1,2-dihydropyridin-4-yl)-4-azaspiro[2.4]heptane-4-carboxylate

To a solution of tert-butyl (R)-6-(1-(2-(2-(2-hydroxyethoxy)ethoxy)ethyl)-2-oxo-1,2-dihydropyridin-4-yl)-4-azaspiro[2.4]heptane-4-carboxylate (400 mg, 0.95 mmol, 1.0 eq.) in DCM (5 mL) were added TEA (192 mg, 1.9 mmol, 2.0 eq.) and MsCl (130 mg, 1.14 mmol, 1.2 eq.) at 0° C. After addition, the resulting mixture was warmed up to room temperature and stirred for 1 hr. After completion, the reaction mixture was diluted with H₂O (10 mL), then extracted with EtOAc (15 mL×3). The organic layers were combined and washed with brine (10 mL), dried over anhydrous Na₂SO₄, and concentrated under reduced pressure. The residue was purified by flash column chromatography on silica gel to afford tert-butyl (R)-6-(1-(2-(2-(2-((methylsulfonyl)oxy)ethoxy)ethoxy)ethyl)-2-oxo-1,2-dihydropyridin-4-yl)-4-azaspiro[2.4]heptane-4-carboxylate (330 mg, 0.66 mmol, 69%) as a colorless oil. LCMS (ESI): m/z=501 [M+H]⁺.

### STEP C: tert-butyl (6R)-6-(1-(2-(2-(2-((2-(2,6-dioxopiperidin-3-yl)-1-oxoisoindoiin-4-yl)oxy)ethoxy)ethoxy)ethyl)-2-oxo-1,2-dihydropyridin-4-yl)-4-azaspiro[2.4]heptane-4-carboxylate

To a solution of tert-butyl (R)-6-(1-(2-(2-(2-((methylsulfonyl)oxy)ethoxy)ethoxy)ethyl)-2-oxo-1,2-dihydropyridin-4-yl)-4-azaspiro[2.4]heptane-4-carboxylate (330 mg, 0.66 mmol, 1.0 eq.) and K₂CO₃ (324 mg, 1.98 mmol, 3.0 eq.) in DMF (5 mL) was added 3-(4-hydroxy-1-oxoisoindolin-2-yl)piperidine-2,6-dione (174 mg, 0.66 mmol, 1.0 eq.) at room temperature. After addition, the resulting mixture was heated to 60° C. and stirred overnight. After completion, the reaction mixture was diluted with H₂O (10 mL), then extracted with EtOAc (15 mL×3). The organic layers were combined and washed with brine (10 mL), dried over anhydrous Na₂SO₄, and concentrated under reduced pressure. The residue was purified by flash column chromatography on silica gel to afford tert-butyl (6R)-6-(1-(2-(2-(2-((2-(2,6-dioxopiperidin-3-yl)-1-oxoisoindolin-4-yl)oxy)ethoxy)ethoxy)ethyl)-2-oxo-1,2-dihydropyridin-4-yl)-4-azaspiro[2.4]heptane-4-carboxylate (340 mg, 0.51 mmol, 77%) as a white solid. LCMS (ESI): m/z=665 [M+H]⁺.

### STEP D: 3-(1-oxo-4-(2-(2-(2-(2-oxo-4-((R)-4-azaspiro[2.4]heptan-6-yl)pyridin-1(2H)-yl)ethoxy)ethoxy)ethoxy)isoindolin-2-yl)piperidine-2,6-dione

To a solution of tert-butyl tert-butyl (6R)-6-(1-(2-(2-(2-((2-(2,6-dioxopiperidin-3-yl)-1-oxoisoindolin-4-yl)oxy)ethoxy)ethoxy)ethyl)-2-oxo-1,2-dihydropyridin-4-yl)-4-azaspiro[2.4]heptane-4-carboxylate (340 mg, 0.51 mmol, 1.0 eq.) in DCM (5 mL) was added TFA (2 mL) and the reaction mixture was stirred at room temperature for 2 hrs under N₂. After completion, the reaction mixture was concentrated under reduced pressure to get crude 3-(1-oxo-4-(2-(2-(2-(2-oxo-4-((R)-4-azaspiro[2.4]heptan-6-yl)pyridin-1(2H)-yl)ethoxy)ethoxy)ethoxy)isoindolin-2-yl)piperidine-2,6-dione (400 mg, quant.) as a yellow oil, which was used in next step directly without further purification. LCMS (ESI): m/z=565 [M+H]⁺.

### STEP E: tert-butyl ((3S,6S,10aS)-3-((6R)-6-(1-(2-(2-(2-((2-(2,6-dioxopiperidin-3-yl)-1-oxoisoindolin-4-yl)oxy)ethoxy)ethoxy)ethyl)-2-oxo-1,2-dihydropyridin-4-yl)-4-azaspiro[2.4]heptane-4-carbonyl)-5-oxodecahydropyrrolo[1,2-a]azocin-6-yl)carbamate

To a solution of (3S,6S,10aS)-6-((tert-butoxycarbonyl)amino)-5-oxodecahydropyrrolo[1,2-a]azocine-3-carboxylic acid (140 mg, 0.43 mmol, 1.2 eq.) and HATU (202 mg, 0.53 mmol, 1.5 eq.) in DMF(2 mL) were added 3-(1-oxo-4-(2-(2-(2-(2-oxo-4-((R)-4-azaspiro[2.4]heptan-6-yl)pyridin-1(2H)-yl)ethoxy)ethoxy)ethoxy)isoindolin-2-yl)piperidine-2,6-dione (200 mg, 0.35 mmol, 1.0 eq.) and DIEA (137 mg, 1.06 mmol, 3.0 eq.). The resulting mixture was stirred at room temperature for 2 hrs under N₂. After completion, the reaction mixture was purified by Biotage® C18 column chromatography to get tert-butyl ((3S,6S,10aS)-3-((6R)-6-(1-(2-(2-(2-((2-(2,6-dioxopiperidin-3-yl)-1-oxoisoindolin-4-yl)oxy)ethoxy)ethoxy)ethyl)-2-oxo-1,2-dihydropyridin-4-yl)-4-azaspiro[2.4]heptane-4-carbonyl)-5-oxodecahydropyrrolo[1,2-a]azocin-6-yl)carbamate (91 mg, 0.11 mmol, 30%) as a white solid. LCMS (ESI): m/z=873 [M+H]⁺.

### STEP F: 3-(4-(2-(2-(2-(4-((R)-4-((3S,6S,10aS)-6-amino-5-oxodecahydropyrrolo[1,2-a]azocine-3-carbonyl)-4-azaspiro[2.4]heptan-6-yl)-2-oxopyridin-1(2H)-yl)ethoxy)ethoxy)ethoxy)-1-oxoisoindolin-2-yl)piperidine-2,6-dione

To a solution of tert-butyl ((3S,6S,10aS)-3-((6R)-6-(1-(2-(2-(2-((2-(2,6-dioxopiperidin-3-yl)-1-oxoisoindolin-4-yl)oxy)ethoxy)ethoxy)ethyl)-2-oxo-1,2-dihydropyridin-4-yl)-4-azaspiro[2.4]heptane-4-carbonyl)-5-oxodecahydropyrrolo[1,2-a]azocin-6-yl)carbamate (60 mg, 69 μmol, 1.0 eq.) in dry DCM (5 mL) was added TFA (1 mL) at 25° C. The mixture was stirred at 25° C. for 2 hrs. After completion, the reaction mixture was concentrated under reduced pressure to give 3-{4-[(8-{4-[(6R)-4-{[(3S,6S,10aS)-6-amino-5-oxodecahydropyrrolo[1,2-a]azocin-3-yl]carbonyl}-4-azaspiro[2.4]heptan-6-yl]-2-oxopyridin-1-yl}-3,6-dioxaoct-1-yl)oxy]-1-oxo-2,3-dihydro-1H-isoindol-2-yl}hexahydropyridine-2,6-dione (50 mg, quant.) as a yellow oil, which was used in next step without further purification. LCMS (ESI): m/z=773.1 [M+H]⁺.

### STEP G: S,S((((2-(((3S,6S,10aS)-3-((6R)-6-(1-(2-(2-(2-((2-(2,6-dioxopiperidin-3-yl)-1-oxoisoindolin-4-yl)oxy)ethoxy)ethoxy)ethyl)-2-oxo-1,2-dihydropyridin-4-yl)-4-azaspiro[2.4]heptane-4-carbonyl)-5-oxodecahydropyrrolo[1,2-a]azocin-6-yl)carbamoyl)benzo[b]thiophen-5-yl)difluoromethyl)phosphoryl)bis(oxy))bis(ethane-2,1-diyl)) dibutanethioate

To a mixture of 3-{4-[(8-{4-[(6R)-4-{[(3S,6S,10aS)-6-amino-5-oxodecahydropyrrolo[1,2-a]azocin-3-yl]carbonyl}-4-azaspiro[2.4]heptan-6-yl]-2-oxopyridin-1-yl}-3,6-dioxaoct-1-yl)oxy]-1-oxo-2,3-dihydro-1H-isoindol-2-yl}hexahydropyridine-2,6-dione (50 mg, 65 μmol, 1.0 eq.) and DIEA (25 mg, 195 μmol, 3.0 eq.) in DMF (3 mL) were added 4-nitrophenyl 5-((bis(2-(butyrylthio)ethoxy)phosphoryl)difluoromethyl)benzo[b]thiophene-2-carboxylate (45 mg, 65 μmol, 1.0 eq.) and DMAP (39 mg, 32 μmol, 0.5 eq.). The mixture was stirred at 25° C. for 30 min. After completion, the reaction mixture was purified by prep-HPLC to give S,S [symbol] (((((2-(((3S,6S,10aS)-3-((6R)-6-(1-(2-(2-(2-((2-(2,6-dioxopiperidin-3-yl)-1-oxoisoindolin-4-yl)oxy)ethoxy)ethoxy)ethyl)-2-oxo-1,2-dihydropyridin-4-yl)-4-azaspiro[2.4]heptane-4-carbonyl)-5-oxodecahydropyrrolo[1,2-a]azocin-6-yl)carbamoyl)benzo[b]thiophen-5-yl)difluoromethyl)phosphoryl)bis(oxy))bis(ethane-2,1-diyl)) dibutanethioate (Compound 26, 31.3 mg, 24 μmol, 37%) as a white solid.

¹H NMR (400 MHz, DMSO-d6) δ 10.96 (s, 1H), 8.94 (d, J=7.1 Hz, 1H), 8.34 (s, 1H), 8.20 (d, J=8.4 Hz, 1H), 8.14 (s, 1H), 7.58 (d, J=8.5 Hz, 1H), 7.53-7.42 (m, 2H), 7.35-7.29 (m, 1H), 7.24-7.16 (m, 1H), 6.30 (s, 1H), 6.20-6.10 (m, 1H), 5.15-5.05 (m, 1H), 5.00-4.89 (m, 1H), 4.52-4.39 (m, 1H), 4.37-4.29 (m, 1H), 4.27-4.10 (m, 8H), 4.05-3.90 (m, 3H), 3.75-3.63 (m, 3H), 3.62-3.56 (m, 2H), 3.49 (s, 4H), 3.16-3.09 (m, 4H), 2.96-2.84 (m, 1H), 2.61-2.53 (m, 4H), 2.46-2.15 (m, 3H), 2.12-1.59 (m, 14H), 1.58-1.46 (m, 7H), 0.85 (t, J=7.4 Hz, 6H), 0.51-0.37 (m, 2H).

LCMS (ESI): m/z=1324.5 [M+H]⁺.

### STEP H: ((2-(((3S,6S,10aS)-3-((6R)-6-(1-(2-(2-(2-((2-(2,6-dioxopiperidin-3-yl)-1-oxoisoindolin-4-yl)oxy)ethoxy)ethoxy)ethyl)-2-oxo-1,2-dihydropyridin-4-yl)-4-azaspiro[2.4]heptane-4-carbonyl)-5-oxodecahydropyrrolo[1,2-a]azocin-6-yl)carbamoyl)benzo[b]thiophen-5-yl)difluoromethyl)phosphonic acid

To a mixture of 3-{4-[(8-{4-[(6R)-4-{[(3S,6S,10aS)-6-amino-5-oxodecahydropyrrolo[1,2-a]azocin-3-yl]carbonyl}-4-azaspiro[2.4]heptan-6-yl]-2-oxopyridin-1-yl}-3,6-dioxaoct-1-yl)oxy]-1-oxo-2,3-dihydro-1H-isoindol-2-yl}hexahydropyridine-2,6-dione (50 mg, 65 μmol, 1.0 eq.) and DIEA (25 mg, 195 μmol, 3.0 eq.) in DMF (3 mL) were added (difluoro(2-((perfluorophenoxy)carbonyl)benzo[b]thiophen-5-yl)methyl)phosphonic acid (31 mg, 65 μmol, 1.0 eq.). The mixture was stirred at 25° C. for 30 min. After completion, the reaction mixture was purified by prep-HPLC to give ((2-(((3S,6S,10aS)-3-((6R)-6-(1-(2-(2-(2-((2-(2,6-dioxopiperidin-3-yl)-1-oxoisoindolin-4-yl)oxy)ethoxy)ethoxy)ethyl)-2-oxo-1,2-dihydropyridin-4-yl)-4-azaspiro[2.4]heptane-4-carbonyl)-5-oxodecahydropyrrolo[1,2-a]azocin-6-yl)carbamoyl)benzo[b]thiophen-5-yl)difluoromethyl)phosphonic acid (Compound 27, 16.2 mg, 15 μmol, 23%) as a white solid.

¹H NMR (400 MHz, DMSO-d6) δ 10.96 (s, 1H), 8.87 (d, J=7.2 Hz, 1H), 8.32 (s, 1H), 8.13 (d, J=8.5 Hz, 1H), 8.07 (s, 1H), 7.58 (d, J=8.6 Hz, 1H), 7.53-7.49 (m, 1H), 7.49-7.43 (m, 1H), 7.33-7.29 (m, 1H), 7.24-7.18 (m, 1H), 6.32-6.28 (m, 1H), 6.17-6.10 (m, 1H), 5.13-5.07 (m, 1H), 4.98-4.92 (m, 1H), 4.48-4.42 (m, 1H), 4.36-4.30 (m, 1H), 4.26-4.19 (m, 2H), 4.19-4.15 (m, 2H), 4.07-3.91 (m, 4H), 3.71-3.63 (m, 3H), 3.61-3.57 (m, 2H), 3.48-3.22 (m, 3H), 2.95-2.84 (m, 1H), 2.63-2.53 (m, 1H), 2.47-2.37 (m, 1H), 2.28-2.19 (m, 1H), 2.05-1.44 (m, 17H), 0.49-0.39 (m, 2H).

LCMS (ESI): m/z=1063.2 [M+H]⁺.

### Preparation of ((2-(((3S,6S,9aS)-3-(3-(4-(4-(2-(4-(2,6-dioxopiperidin-3-yl)phenoxy)acetyl)piperazin-1-yl)pyridin-3-yl)azetidine-1-carbonyl)-5-oxooctahydro-1H-pyrrolo[1,2-a]azepin-6-yl)carbamoyl)benzo[b]thiophen-5-yl)difluoromethyl)phosphonic acid (Compound 28) and S,S(((((2-(((3S,6S,9aS)-3-(3-(4-(4-(2-(4-(2,6-dioxopiperidin-3-yl)phenoxy)acetyl)piperazin-1-yl)pyridin-3-yl)azetidine-1-carbonyl)-5-oxooctahydro-1H-pyrrolo[1,2-a]azepin-6-yl)carbamoyl)benzo[b]thiophen-5-yl)difluoromethyl)phosphoryl)bis(oxy))bis(ethane-2,1-diyl)) dibutanethioate (Compound 29)

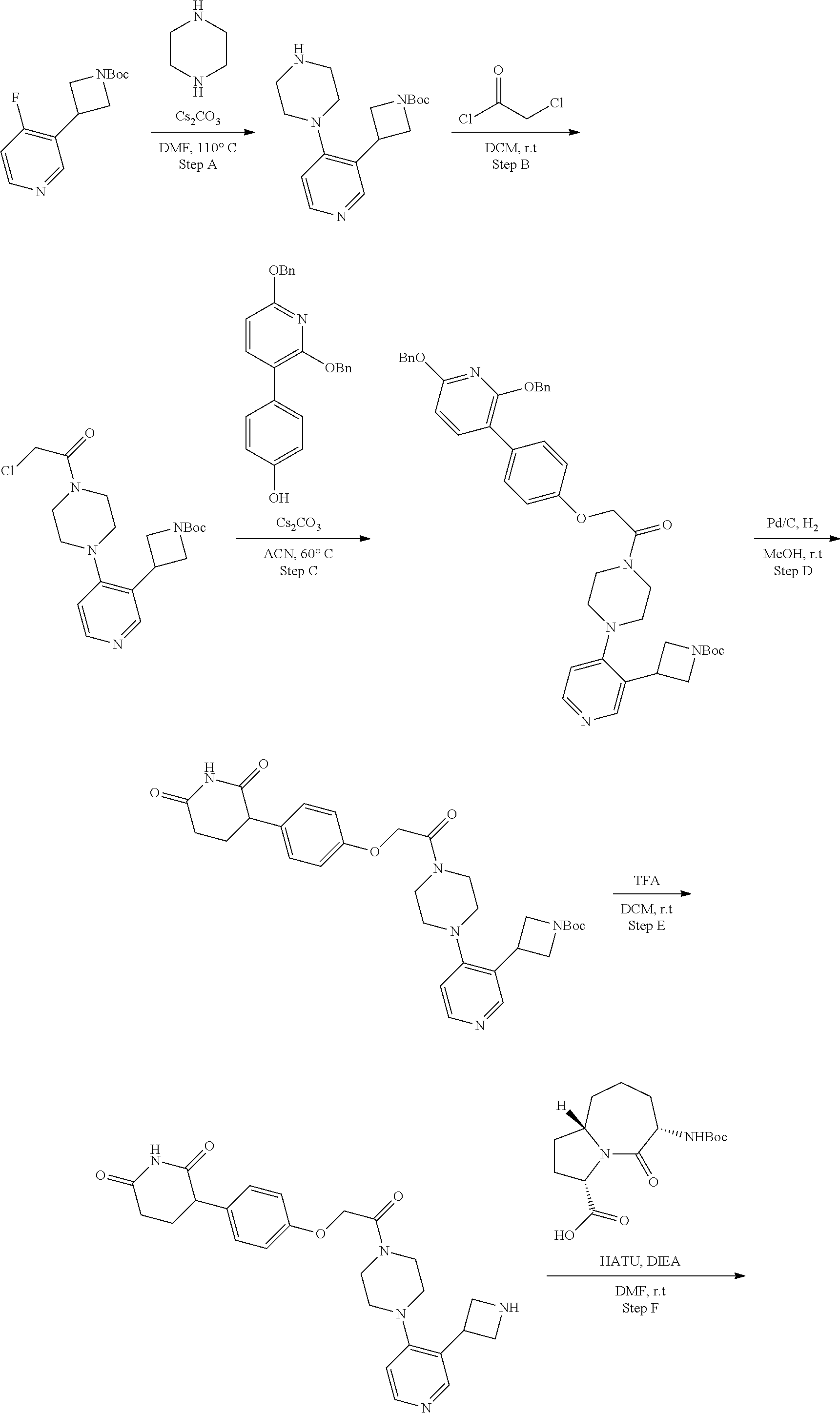

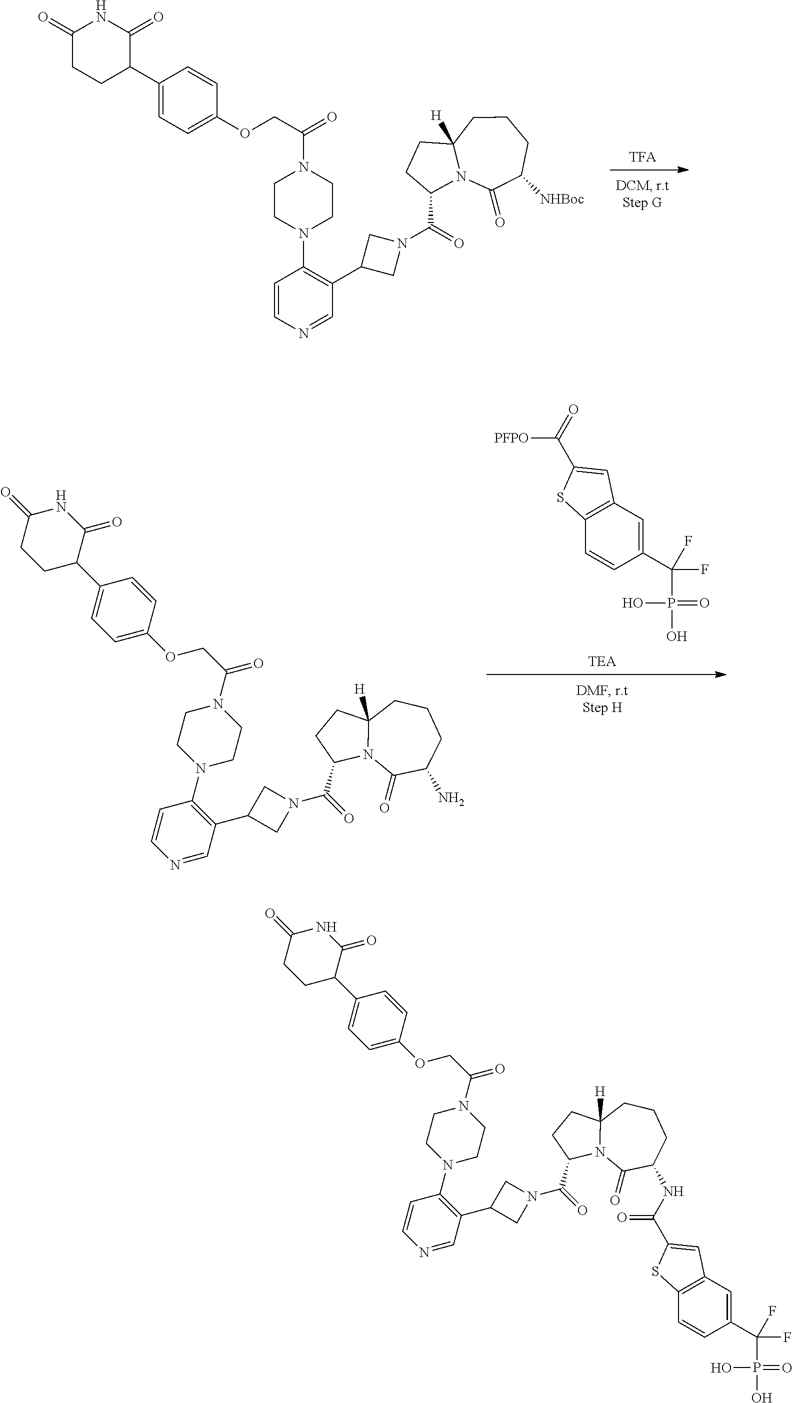

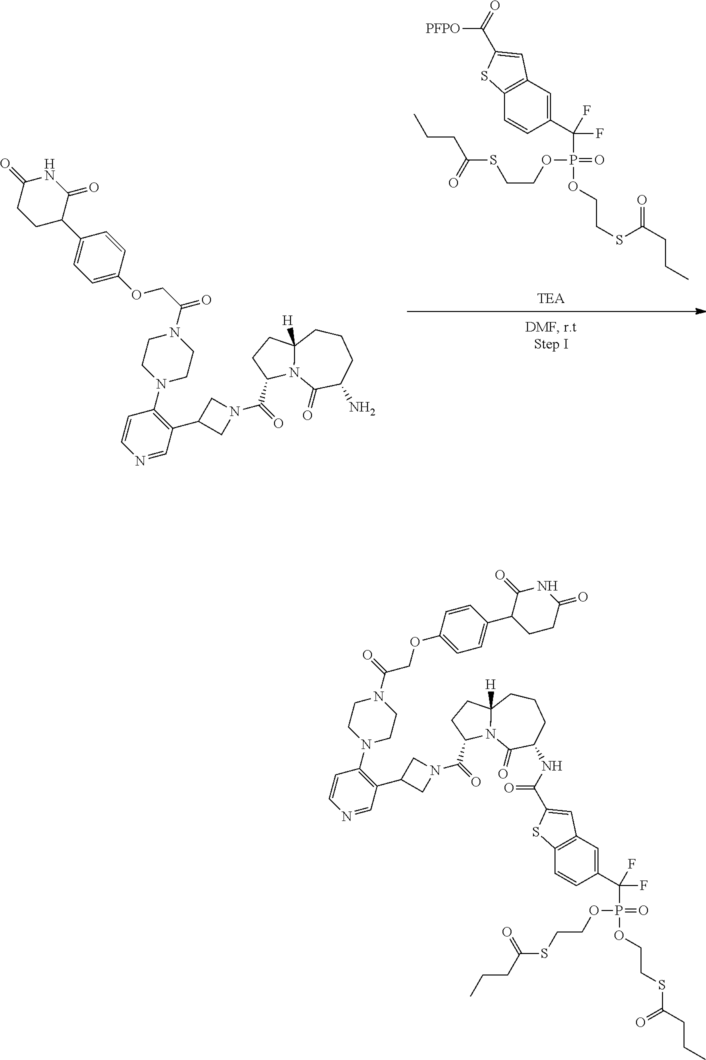

### STEP A tert-butyl 3-(4-(piperazin-1-yl)pyridin-3-yl)azetidine-1-carboxylate

To a solution of tert-butyl 3-(4-fluoropyridin-3-yl)azetidine-1-carboxylate (1.0 g, 3.97 mmol, 1.0 eq.) in DMF (10 mL) were added piperazine (0.68 g, 7.94 mmol, 2.0 eq.) and Cs₂CO₃ (2.58 g, 7.94 mmol, 2.0 eq.). The resulting mixture was stirred at 110° C. overnight. After completion, The residue was purified by Biotage® C18 column chromatography to get tert-butyl 3-(4-(piperazin-1-yl)pyridin-3-yl)azetidine-1l-carboxylate (0.9 g, 2.82 mmol, 71%) as a white solid. LCMS (ESI): m/z=319 [M+H]+

### STEP B: tert-butyl 3-(4-(4-(2-chloroacetyl)piperazin-1-yl)pyridin-3-yl)azetidine-1-carboxylate

To a solution of tert-butyl 3-(4-(piperazin-1-yl)pyridin-3-yl)azetidine-1-carboxylate (0.9 g, 2.82 mmol, 1.0 eq.) in DCM (20 mL) was added 2-chloroacetyl chloride (0.35 g, 3.1 mmol, 1.1 eq.) and the resulting mixture was stirred at room temperature for 2 hrs under N₂. After completion, the reaction mixture was concentrated under reduced pressure. The residue was purified by Biotage® C18 column chromatography to get tert-butyl 3-(4-(piperazin-1-yl)pyridin-3-yl)azetidine-1-carboxylate (0.5 g, 1.26 mmol, 45%) as a white solid. LCMS (ESI): m/z=395 [M+H]⁺.

### STEP C: tert-butyl 3-(4-(4-(2-(4-(2,6-bis(benzyloxy)pyridin-3-yl)phenoxy)acetyl)piperazin-1-yl)pyridin-3-yl)azetidine-1-carboxylate

To a solution of tert-butyl 3-(4-(piperazin-1-yl)pyridin-3-yl)azetidine-1-carboxylate (0.5 g, 1.26 mmol, 1 eq) in ACN (5 mL) were added 4-(2,6-bis(benzyloxy)pyridin-3-yl)phenol (483 mg, 1.26 mmol, 1.0 eq.) and Cs₂CO₃ (822 mg, 2.52 mmol, 2 eq.). The resulting mixture was stirred at 60° C. for 6 hrs under N₂. After completion, the reaction mixture was concentrated under reduced pressure. The residue was purified by Biotage® C18 column chromatography to get tert-butyl 3-(4-(4-(2-(4-(2,6-bis(benzyloxy)pyridin-3-yl)phenoxy)acetyl)piperazin-1-yl)pyridin-3-yl)azetidine-1-carboxylate (450 mg, 0.62 mmol, 49%) as a white solid. LCMS (ESI): m/z=742 [M+H]⁺.

### STEP D: tert-butyl 3-(4-(4-(2-(4-(2,6-dioxopiperidin-3-yl)phenoxy)acetyl)piperazin-1-yl)pyridin-3-yl)azetidine-1-carboxylate

To a solution of tert-butyl 3-(4-(4-(2-(4-(2,6-bis(benzyloxy)pyridin-3-yl)phenoxy)acetyl)piperazin-1-yl)pyridin-3-yl)azetidine-1-carboxylate (450 mg, 0.62 mmol, 1.0 eq.) in MeOH (20 mL) was added Pd/C (20 mg) under nitrogen. The suspension was degassed under vacuum and purged with H₂ several times. The resulting mixture was stirred at room temperature for 3 hrs. After completion, the suspension was filtered through a pad of Celite®, the filter cake was washed with MeOH (10 mL). The combined filtrates were concentrated to dryness to give tert-butyl 3-(4-(4-(2-(4-(2,6-dioxopiperidin-3-yl)phenoxy)acetyl)piperazin-1-yl)pyridin-3-yl)azetidine-1-carboxylate (200 mg, 0.35 mmol, 58%) as a white solid. LCMS (ESI): m/z=564 [M+H]⁺.

### STEP E: 3-(4-(2-(4-(3-(azetidin-3-yl)pyridin-4-yl)piperazin-1-yl)-2-oxoethoxy)phenyl)piperidine-2,6-dione

To a solution of tert-butyl 3-(4-(4-(2-(4-(2,6-dioxopiperidin-3-yl)phenoxy)acetyl)piperazin-1-yl)pyridin-3-yl)azetidine-1-carboxylate (200 mg, 0.35 mmol, 1.0 eq.) in DCM (10 mL) was added TFA (1 mL) and the reaction mixture was stirred at room temperature for 4 hrs under N₂. After completion, the reaction mixture was concentrated under reduced pressure to get crude 3-(4-(2-(4-(3-(azetidin-3-yl)pyridin-4-yl)piperazin-1-yl)-2-oxoethoxy)phenyl)piperidine-2,6-dione (180 mg, quant.) as a white solid, which was used in next step directly without further purification. LCMS (ESI): m/z=464 [M+H]⁺.

### STEP F: tert-butyl ((3S,6S,9aS)-3-(3-(4-(4-(2-(4-(2,6-dioxopiperidin-3-yl)phenoxy)acetyl)piperazin-1-yl)pyridin-3-yl)azetidine-1-carbonyl)-5-oxooctahydro-1H-pyrrolo[1,2-a]azepin-6-yl)carbamate

To a solution of (3S,6S,9aS)-6-((tert-butoxycarbonyl)amino)-5-oxooctahydro-1H-pyrrolo[1,2-a]azepine-3-carboxylic acid (60 mg, 0.19 mmol, 1 eq.) and HATU (96 mg, 0.25 mmol, 1.3 eq.) in DMF (2 mL) were added 3-(4-(2-(4-(3-(azetidin-3-yl)pyridin-4-yl)piperazin-1-yl)-2-oxoethoxy)phenyl)piperidine-2,6-dione (90 mg, 0.19 mmol, 1.0 eq.) and DIEA (74 mg, 0.57 mmol, 3 eq.). The resulting mixture was stirred at room temperature for 2 hrs under N₂. After completion, the reaction mixture was purified by Biotage® C18 column chromatography to get tert-butyl ((3S,6S,9aS)-3-(3-(4-(4-(2-(4-(2,6-dioxopiperidin-3-yl)phenoxy)acetyl)piperazin-1-yl)pyridin-3-yl)azetidine-1-carbonyl)-5-oxooctahydro-1H-pyrrolo[1,2-a]azepin-6-yl)carbamate (40 mg, 58 μmol, 28%) as a white solid. LCMS (ESI): m/z=758 [M+H]⁺.

### STEP G: 3-(4-(2-(4-(3-(1-((3S,6S,9aS)-6-amino-5-oxooctahydro-1H-pyrrolo[1,2-a]azepine-3-carbonyl)azetidin-3-yl)pyridin-4-yl)piperazin-1-yl)-2-oxoethoxy)phenyl)piperidine-2,6-dione

To a solution of tert-butyl ((3S,6S,9aS)-3-(3-(4-(4-(2-(4-(2,6-dioxopiperidin-3-yl)phenoxy)acetyl)piperazin-1-yl)pyridin-3-yl)azetidine-1-carbonyl)-5-oxooctahydro-1H-pyrrolo[1,2-a]azepin-6-yl)carbamate (40 mg, 58 umol, 1.0 eq.) in DCM (5 mL) was added TFA (1 mL) and the reaction mixture was stirred at room temperature for 4 hrs under N₂. After completion, the reaction mixture was concentrated under reduced pressure to get crude 3-(4-(2-(4-(3-(1-((3S,6S,9aS)-6-amino-5-oxooctahydro-1H-pyrrolo[1,2-a]azepine-3-carbonyl)azetidin-3-yl)pyridin-4-yl)piperazin-1-yl)-2-oxoethoxy)phenyl)piperidine-2,6-dione (40 mg, quant.) as a white solid, which was used in next step directly without further purification. LCMS (ESI): m/z=658 [M+H]⁺.

### STEP H: ((2-(((3S,6S,9aS)-3-(3-(4-(4-(2-(4-(2,6-dioxopiperidin-3-yl)phenoxy)acetyl)piperazin-1-yl)pyridin-3-yl)azetidine-1-carbonyl)-5-oxooctahydro-1H-pyrrolo[1,2-a]azepin-6-yl)carbamoyl)benzo[b]thiophen-5-yl)difluoromethyl)phosphonic acid

To a solution of 3-(4-(2-(4-(3-(1-((3S,6S,9aS)-6-amino-5-oxooctahydro-1H-pyrrolo[1,2-a]azepine-3-carbonyl)azetidin-3-yl)pyridin-4-yl)piperazin-1-yl)-2-oxoethoxy)phenyl)piperidine-2,6-dione (20 mg, 30 μmol, 1.0 eq.) in DMF (1 mL) were added (difluoro(2-((perfluorophenoxy)carbonyl)benzo[b]thiophen-5-yl)methyl)phosphonic acid (15 mg, 30 μmol, 1.0 eq.) and TEA (9 mg, 90 μmol, 3.0 eq.). The resulting mixture was stirred at room temperature for 2 hrs under N₂. After completion, the reaction mixture was purified by prep-HPLC to get ((2-(((3S,6S,9aS)-3-(3-(4-(4-(2-(4-(2,6-dioxopiperidin-3-yl)phenoxy)acetyl)piperazin-1-yl)pyridin-3-yl)azetidine-1-carbonyl)-5-oxooctahydro-1H-pyrrolo[1,2-a]azepin-6-yl)carbamoyl)benzo[b]thiophen-5-yl)difluoromethyl)phosphonic acid (Compound 28, 8 mg, 8.4 μmol, 28%) as a white solid.

¹H NMR (400 MHz, DMSO-d6) δ 10.79 (s, 1H), 8.83-8.68 (m, 1H), 8.61-8.34 (m, 2H), 8.32-8.21 (m, 1H), 8.17-8.01 (m, 2H), 7.64-7.54 (m, 1H), 7.35-7.22 (m, 1H), 7.12 (t, J=7.7 Hz, 2H), 6.89 (t, J=8.1 Hz, 2H), 4.93-4.78 (m, 3H), 4.72-4.61 (m, 2H), 4.49-4.29 (m, 5H), 4.22-4.14 (m, 2H), 4.01-3.98 (m, 2H), 3.80-3.76 (m, 2H), 3.67-3.62 (m, 4H), 3.45-3.40 (m, 2H), 2.71-2.62 (m, 1H), 2.27-2.11 (m, 2H), 2.06-1.92 (m, 3H), 1.92-1.58 (m, 7H).

LCMS (ESI): m/z=948.5 [M+H]⁺.

### STEP I: S,S(((((2-(((3S,6S,9aS)-3-(3-(4-(4-(2-(4-(2,6-dioxopiperidin-3-yl)phenoxy)acetyl)piperazin-1-yl)pyridin-3-yl)azetidine-1-carbonyl)-5-oxooctahydro-1H-pyrrolo[1,2-a]azepin-6-yl)carbamoyl)benzo[b]thiophen-5-yl)difluoromethyl)phosphoryl)bis(oxy))bis(ethane-2,1-diyl)) dibutanethioate

To a solution of 3-(4-(2-(4-(3-(1-((3S,6S,9aS)-6-amino-5-oxooctahydro-1H-pyrrolo[1,2-a]azepine-3-carbonyl)azetidin-3-yl)pyridin-4-yl)piperazin-1-yl)-2-oxoethoxy)phenyl)piperidine-2,6-dione (20 mg, 30 μmol, 1.0 eq.) in DMF (1 mL) were added 4-nitrophenyl 5-((bis(2-(butyrylthio)ethoxy)phosphoryl)difluoromethyl)benzo[b]thiophene-2-carboxylate (21 mg, 30 μmol, 1.0 eq.) and TEA (9 mg, 90 μmol, 3.0 eq.). The resulting mixture was stirred at room temperature for 2 hrs under N₂. After completion, the reaction mixture was concentrated under reduced pressure. The residue was purified by prep-HPLC to get S,S [symbol] (((((2-(((3S,6S,9aS)-3-(3-(4-(4-(2-(4-(2,6-dioxopiperidin-3-yl)phenoxy)acetyl)piperazin-1-yl)pyridin-3-yl)azetidine-1-carbonyl)-5-oxooctahydro-1H-pyrrolo[1,2-a]azepin-6-yl)carbamoyl)benzo[b]thiophen-5-yl)difluoromethyl)phosphoryl)bis(oxy))bis(ethane-2,1-diyl)) dibutanethioate (Compound 29, 7 mg, 6 μmol, 33%) as a white solid.

¹H NMR (400 MHz, DMSO-d6) δ 10.79 (s, 1H), 8.88-8.74 (m, 1H), 8.61-8.41 (m, 2H), 8.31 (d, J=14.7 Hz, 1H), 8.24-8.11 (m, 2H), 7.59 (d, J=8.7 Hz, 1H), 7.33 (t, J=7.0 Hz, 1H), 7.12 (t, J=7.9 Hz, 2H), 6.90 (t, J=7.9 Hz, 2H), 4.90-4.62 (m, 4H), 4.48-4.29 (m, 3H), 4.24-4.10 (m, 5H), 4.04-3.97 (m, 2H), 3.84-3.59 (m, 10H), 3.14 (t, J=6.1 Hz, 4H), 2.71-2.61 (m, 1H), 2.54 (t, J=7.3 Hz, 4H), 2.26-2.11 (m, 2H), 2.05-1.93 (m, 3H), 1.91-1.64 (m, 7H), 1.59-1.51 (m, 4H), 0.86 (t, J=7.5 Hz, 6H).

LCMS (ESI): m/z=1208.4 [M+H]⁺.

### Preparation of ((2-(((3S,6S,10aR)-3-(3-(3-(2-(2-(2-((2-(2,6-dioxopiperidin-3-yl)-1,3-dioxoisoindolin-4-yl)oxy)acetamido)ethoxy)ethoxy)propanamido)-3-(pyridin-2-yl)azetidine-1-carbonyl)-5-oxodecahydropyrrolo[1,2-a]azocin-6-yl)carbamoyl)benzo[b]thiophen-5-yl)difluoromethyl)phosphonic acid (Compound 30)

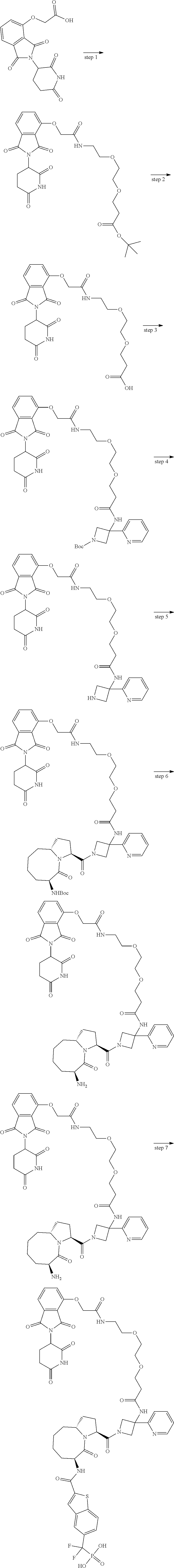

### Step 1: Preparation of tert-butyl 3-(2-(2-(2-((2-(2,6-dioxopiperidin-3-yl)-1,3-dioxoisoindolin-4-yl)oxy)acetamido)ethoxy)ethoxy)propanoate

To a solution of 2-{[2-(2,6-dioxopiperidin-3-yl)-1,3-dioxo-2,3-dihydro-1H-isoindol-4-yl]oxy}acetic acid (0.1 g, 0.3 mmol, 1 eq) in dimethylformamide (1.0 mL) was added HATU (0.17 g, 0.45 mmol, 1.5 eq) and ethylbis(propan-2-yl)amine (0.12 g, 0.9 mmol, 3.0 eq), the mixture was stirred at 25° C. for 15 min, then a solution of tert-butyl 3-[2-(2-aminoethoxy)ethoxy]propanoate (70 mg, 0.3 mmol, 1 eq) and ethylbis(propan-2-yl)amine (0.12 g, 0.9 mmol, 3.0 eq) in dimethylformamide (1.0 mL) was added to the mixture, the mixture was stirred at 25° C. for 30 min to give yellow solution. The residue was diluted with water (5 mL) and extracted with ethyl acetate (5 mL×2), the combined organic layers were washed with saturated brine (5 mL×2), dried over Na₂SO₄, filtered and concentrated under reduced pressure to give a residue. The residue was purified by column chromatography to give tert-butyl 3-{2-[2-(2-{[2-(2,6-dioxopiperidin-3-yl)-1,3-dioxo-2,3-dihydro-1H-isoindol-4-yl]oxy}acetamido)ethoxy]ethoxy}propanoate (0.3 g, 0.5 mmol, 18% yield) was obtained as a yellow oil. LCMS (ESI) m/z=548.2

### Step 2: Preparation of 3-(2-(2-(2-((2-(2,6-dioxopiperidin-3-yl)-1,3-dioxoisoindolin-4-yl)oxy)acetamido)ethoxy)ethoxy)propanoic acid

To a solution of tert-butyl 3-{2-[2-(2-{[2-(2,6-dioxopiperidin-3-yl)-1,3-dioxo-2,3-dihydro-1H-isoindol-4-yl]oxy}acetamido)ethoxy]ethoxy}propanoate (0.3 g, 0.5 mmol, 1 eq) in methylene chloride (3 mL) was added trifluoroacetic acid (1 mL), the mixture was stirred at 25° C. for 15 minutes to give yellow solution. The reaction mixture was concentrated under reduced pressure to give 3-{2-[2-(2-{[2-(2,6-dioxopiperidin-3-yl)-1,3-dioxo-2,3-dihydro-1H-isoindol-4-yl]oxy}acetamido)ethoxy]ethoxy}propanoic acid (0.3 g, crude) as a yellow oil.

LCMS (ESI) m/z=448.2

### Step 3: Preparation of tert-butyl 3-(3-(2-(2-(2-((2-(2,6-dioxopiperidin-3-yl)-1,3-dioxoisoindolin-4-yl)oxy)acetamido)ethoxy)ethoxy)propanamido)-3-(pyridin-2-yl)azetidine-1-carboxylate

To a solution of 3-{2-[2-(2-{[2-(2,6-dioxopiperidin-3-yl)-1,3-dioxo-2,3-dihydro-1H-isoindol-4-yl]oxy}acetamido)ethoxy]ethoxy}propanoic acid (0.15 g, 0.31 mmol, 1 eq) in dimethylformamide (1.5 mL) was added HATU (0.17 g, 0.46 mmol, 1.5 eq) and N,N-diisopropylethylamine (0.12 g, 0.92 mmol, 3.0 eq), the mixture was stirred at 25° C. for 5 min, then a solution of tert-butyl 3-amino-3-(pyridin-2-yl)azetidine-1-carboxylate (76 mg, 0.31 mmol, 1 eq) and N,N-diisopropylethylamine (0.12 g, 0.92 mmol, 3.0 eq) in dimethylformamide (1.0 mL) was added to the mixture, the mixture was stirred at 25° C. for 30 min to give yellow solution. The residue was purified by reversed phase (TFA condition) to give tert-butyl 3-(3-{2-[2-(2-{[2-(2,6-dioxopiperidin-3-yl)-1,3-dioxo-2,3-dihydro-1H-isoindol-4-yl]oxy}acetamido)ethoxy]ethoxy}propanamido)-3-(pyridin-2-yl)azetidine-1-carboxylate (80 mg, 0.11 mmol, 36% yield) was obtained as a yellow oil. LCMS (ESI) m/z=723.3

### Step 4: Preparation of 3-(2-(2-(2-((2-(2,6-dioxopiperidin-3-yl)-1,3-dioxoisoindolin-4-yl)oxy)acetamido)ethoxy)ethoxy)-N-(3-(pyridin-2-yl)azetidin-3-yl)propanamide

To a solution of tert-butyl 3-(3-{2-[2-(2-{[2-(2,6-dioxopiperidin-3-yl)-1,3-dioxo-2,3-dihydro-1H-isoindol-4-yl]oxy}acetamido)ethoxy]ethoxy}propanamido)-3-(pyridin-2-yl)azetidine-1-carboxylate (80 mg, 0.11 mmol, 1 eq) in methylene chloride (1.0 mL) was added trifluoroacetic acid (0.3 mL), the mixture was stirred at 25° C. for 15 min to give yellow solution. The reaction mixture was concentrated under reduced pressure to give 3-{2-[2-(2-{[2-(2,6-dioxopiperidin-3-yl)-1,3-dioxo-2,3-dihydro-1H-isoindol-4-yl]oxy}acetamido)ethoxy]ethoxy}-N-[3-(pyridin-2-yl)azetidin-3-yl]propanamide (80 mg, crude) was obtained as a yellow oil. LCMS (ESI) m/z=623.3

Step 5: Preparation of tert-butyl ((3S,6S,10aR)-3-(3-(3-(2-(2-(2-((2-(2,6-dioxopiperidin-3-yl)-1,3-dioxoisoindolin-4-yl)oxy)acetamido)ethoxy)ethoxy)propanamido)-3-(pyridin-2-yl)azetidine-1-carbonyl)-5-oxodecahydropyrrolo[1,2-a]azocin-6-yl)carbamate

To a solution of 3-{2-[2-(2-{[2-(2,6-dioxopiperidin-3-yl)-1,3-dioxo-2,3-dihydro-1H-isoindol-4-yl]oxy}acetamido)ethoxy]ethoxy}-N-[3-(pyridin-2-yl)azetidin-3-yl]propanamide (70 mg, 0.11 mmol, 1 eq) in dimethylformamide (1.0 mL) was added N,N-diisopropylethylamine (43 mg, 0.34 mmol, 3.0 eq), the mixture was stirred at 25° C. for 15 min, then a solution of 3-{2-[2-(2-{[2-(2,6-dioxopiperidin-3-yl)-1,3-dioxo-2,3-dihydro-1H-isoindol-4-yl]oxy}acetamido)ethoxy]ethoxy}-N-[3-(pyridin-2-yl)azetidin-3-yl]propanamide (70 mg, 0.11 mmol, 1 eq), 1-[(dimethylamino)(dimethyliminiumyl)methyl]-3-oxo-1H,2H,3H-3λ⁵-[1,2,3]triazolo[5,4-b]pyridin-3-ylium-2-ide; hexafluoro-λ⁵-phosphanuide (64 mg, 0.17 mmol, 1.5 eq) and N,N-diisopropylethylamine (43 mg, 0.34 mmol, 3.0 eq) in dimethylformamide (1.0 mL) was added to the mixture, the mixture was stirred at 25° C. for 30 min to give yellow solution. The residue was purified by reversed phase (TFA) to give tert-butyl N-[(3S,6S,10aR)-3-[3-(3-{2-[2-(2-{[2-(2,6-dioxopiperidin-3-yl)-1,3-dioxo-2,3-dihydro-1H-isoindol-4-yl]oxy}acetamido)ethoxy]ethoxy}propanamido)-3-(pyridin-2-yl)azetidine-1-carbonyl]-5-oxo-decahydropyrrolo[1,2-a]azocin-6-yl]carbamate (50 mg, 0.054 mmol, 48% yield) was obtained as a yellow oil. LCMS (ESI) m/z=931.4

### Step 6: Preparation of N-(1-((3S,6S,10aR)-6-amino-5-oxodecahydropyrrolo[1,2-a]azocine-3-carbonyl)-3-(pyridin-2-yl)azetidin-3-yl)-3-(2-(2-(2-((2-(2,6-dioxopiperidin-3-yl)-1,3-dioxoisoindolin-4-yl)oxy)acetamido)ethoxy)ethoxy)propanamide

To a solution of tert-butyl N-[(3S,6S,10aR)-3-[3-(3-{2-[2-(2-{[2-(2,6-dioxopiperidin-3-yl)-1,3-dioxo-2,3-dihydro-1H-isoindol-4-yl]oxy}acetamido)ethoxy]ethoxy}propanamido)-3-(pyridin-2-yl)azetidine-1-carbonyl]-5-oxo-decahydropyrrolo[1,2-a]azocin-6-yl]carbamate (45 mg, 48 μmol, 1 eq) in methylene chloride (0.9 mL) was added trifluoroacetic acid (0.3 mL), the mixture was stirred at 25° C. for 15 min to give yellow solution. The reaction mixture was concentrated under reduced pressure to give N-{1-[(3S,6S,10aR)-6-amino-5-oxo-decahydropyrrolo[1,2-a]azocine-3-carbonyl]-3-(pyridin-2-yl)azetidin-3-yl}-3-{2-[2-(2-{[2-(2,6-dioxopiperidin-3-yl)-1,3-dioxo-2,3-dihydro-1H-isoindol-4-yl]oxy}acetamido)ethoxy]ethoxy}propanamide (45 mg, crude) was obtained as a yellow oil. LCMS (ESI) m/z=831.4

### Step 7: Preparation of ((2-(((3S,6S,10aR)-3-(3-(3-(2-(2-(2-((2-(2,6-dioxopiperidin-3-yl)-1,3-dioxoisoindolin-4-yl)oxy)acetamido)ethoxy)ethoxy)propanamido)-3-(pyridin-2-yl)azetidine-1-carbonyl)-5-oxodecahydropyrrolo[1,2-a]azocin-6-yl)carbamoyl)benzo[b]thiophen-5-yl)difluoromethyl)phosphonic acid

To a solution of N-{1-[(3S,6S,10aR)-6-amino-5-oxo-decahydropyrrolo[1,2-a]azocine-3-carbonyl]-3-(pyridin-2-yl)azetidin-3-yl}-3-{2-[2-(2-{[2-(2,6-dioxopiperidin-3-yl)-1,3-dioxo-2,3-dihydro-1H-isoindol-4-yl]oxy}acetamido)ethoxy]ethoxy}propanamide (40 mg, 48 μmol, 1 eq) and [difluoro({2-[(4-nitrophenoxy)carbonyl]-1-benzothiophen-5-yl})methyl]phosphonic acid (21 mg, 48 μmol, 1 eq) in methylene chloride (1.0 mL) was added 1H-1,2,3-benzotriazol-3-ium-1-olate amine (11 mg, 72 μmol, 1.5 eq) and N,N-diisopropylethylamine (19 mg, 0.14 mmol, 3.0 eq), the mixture was stirred at 25° C. for 15 min to give yellow solution. The residue was purified by prep-HPLC (column: Phenomenex Luna C18 150×25 mm×10 um, mobile phase: water (0.225% FA)-ACN; B %: 30%-45%, 8 min) then lyophilization to give [(2-{[(3S,6S,10aR)-3-[3-(3-{2-[2-(2-{[2-(2,6-dioxopiperidin-3-yl)-1,3-dioxo-2,3-dihydro-1H-isoindol-4-yl]oxy}acetamido)ethoxy]ethoxy}propanamido)-3-(pyridin-2-yl)azetidine-1-carbonyl]-5-oxo-decahydropyrrolo[1,2-a]azocin-6-yl]carbamoyl}-1-benzothiophen-5-yl)difluoromethyl]phosphonic acid (Compound 30, 21.3 mg, 19 μmol, 40% yield) was obtained as a white solid. LCMS (ESI) m/z=1121.3

¹H NMR (400 MHz, DMSO-d₆) δ 11.11 (s, 1H), 9.18-9.06 (m, 1H), 8.87 (d, J=7.2 Hz, 1H), 8.65-8.56 (m, 1H), 8.32 (s, 1H), 8.14 (d, J=8.4 Hz, 1H), 8.07 (s, 1H), 8.03-7.95 (m, 1H), 7.83-7.70 (m, 2H), 7.58 (d, J=8.8 Hz, 1H), 7.51-7.43 (m, 1H), 7.40 (d, J=4.4 Hz, 1H), 7.38-7.35 (m, 1H), 5.15-5.07 (m, 1H), 4.97-4.90 (m, 1H), 4.77 (d, J=5.2 Hz, 2H), 4.61-4.53 (m, 2H), 4.39-4.22 (m, 4H), 4.18-4.16 (m, 1H), 4.10-4.01 (m, 1H), 3.66-3.59 (m, 2H), 3.53-3.49 (m, 3H), 3.48-3.41 (m, 2H), 3.33-3.25 (m, 2H), 2.94-2.82 (m, 1H), 2.68-2.52 (m, 5H), 2.47-2.42 (m, 1H), 2.23-2.11 (m, 2H), 2.07-1.97 (m, 2H), 1.95-1.77 (m, 6H), 1.69-1.45 (m, 3H).

### Preparation of (difluoro(2-(((3S,6S,10aR)-3-(3-(2-(2-(2-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-2-oxoethoxy)ethoxy)acetamido)-3-(pyridin-2-yl)azetidine-1-carbonyl)-5-oxodecahydropyrrolo[1,2-a]azocin-6-yl)carbamoyl)benzo[b]thiophen-5-yl)methyl)phosphonic acid (Compound 31)

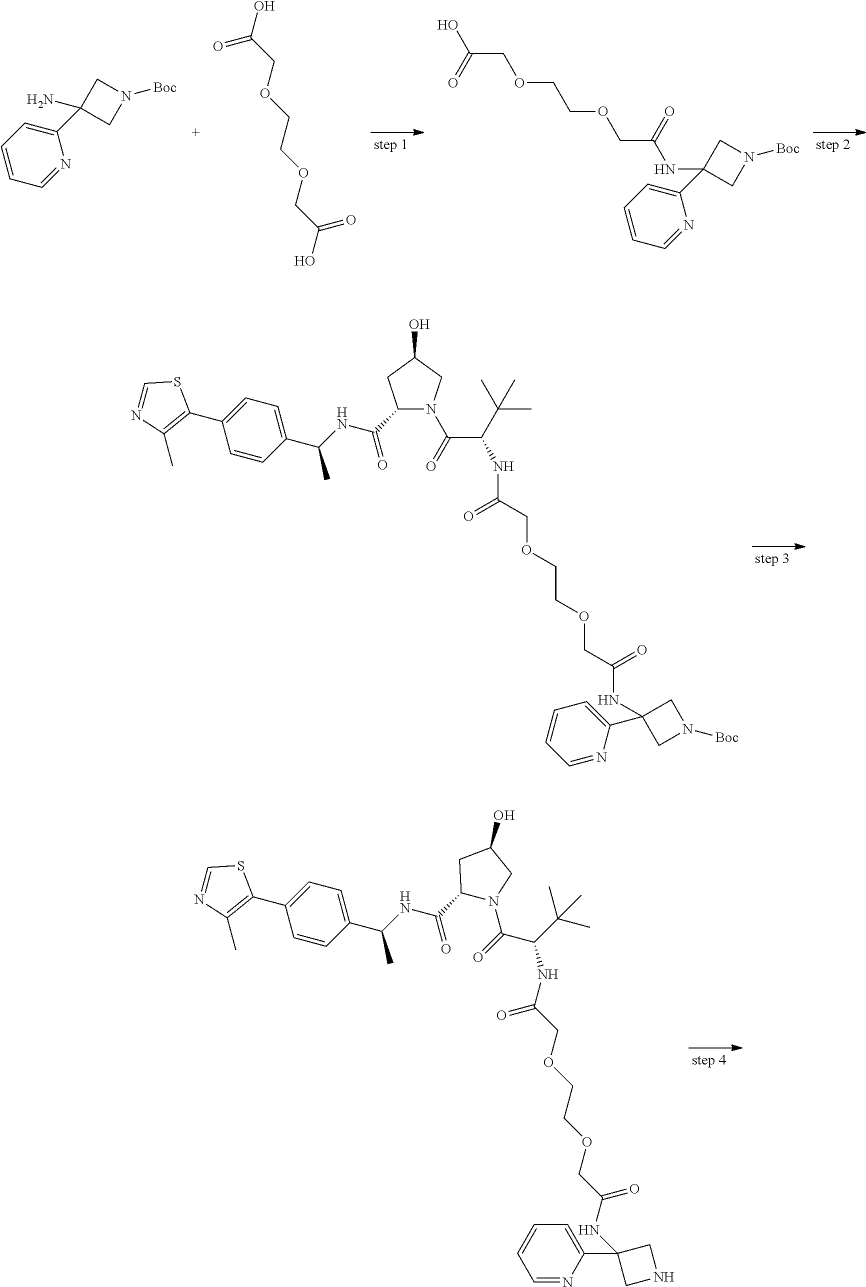 [SMILES: CC(C)(C)OC(=O)N1CC(N)(c2ccccn2)C1 ; O=C(O)COCCOCC(=O)O ; CC(C)(C)OC(=O)N1CC(NC(=O)COCCOCC(=O)O)(c2ccccn2)C1 ; Cc1ncsc1-c1ccc([C@H](C)NC(=O)[C@@H]2C[C@@H](O)CN2C(=O)[C@@H](NC(=O)COCCOCC(=O)NC2(c3ccccn3)CN(C(=O)OC(C)(C)C)C2)C(C)(C)C)cc1 ; Cc1ncsc1-c1ccc([C@H](C)NC(=O)[C@@H]2C[C@@H](O)CN2C(=O)[C@@H](NC(=O)COCCOCC(=O)NC2(c3ccccn3)CNC2)C(C)(C)C)cc1]

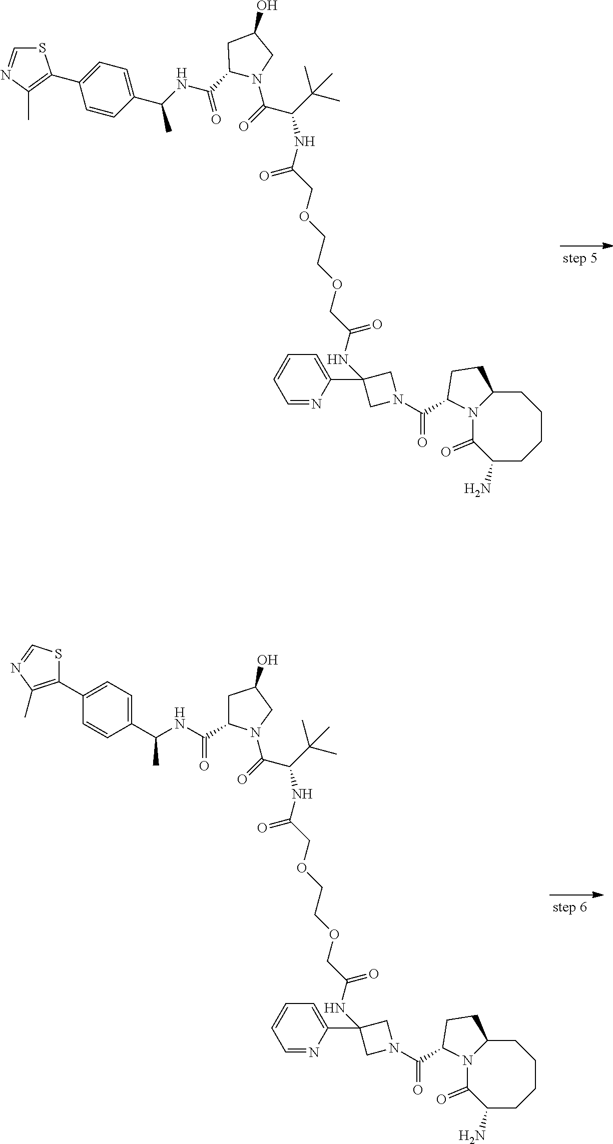 [SMILES: Cc1ncsc1-c1ccc([C@H](C)NC(=O)[C@@H]2C[C@@H](O)CN2C(=O)[C@@H](NC(=O)COCCOCC(=O)NC2(c3ccccn3)CN(C(=O)[C@@H]3CC[C@H]4CCCC[C@H](N)C(=O)N43)C2)C(C)(C)C)cc1 ; Cc1ncsc1-c1ccc([C@H](C)NC(=O)[C@@H]2C[C@@H](O)CN2C(=O)[C@@H](NC(=O)COCCOCC(=O)NC2(c3ccccn3)CN(C(=O)[C@@H]3CC[C@H]4CCCC[C@H](N)C(=O)N43)C2)C(C)(C)C)cc1]

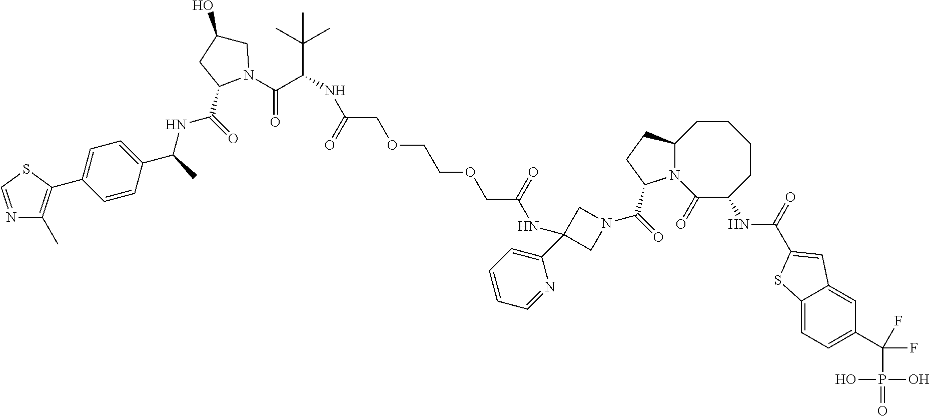 [SMILES: Cc1ncsc1-c1ccc([C@H](C)NC(=O)[C@@H]2C[C@@H](O)CN2C(=O)[C@@H](NC(=O)COCCOCC(=O)NC2(c3ccccn3)CN(C(=O)[C@@H]3CC[C@H]4CCCC[C@H](NC(=O)c5cc6cc(C(F)(F)P(=O)(O)O)ccc6s5)C(=O)N43)C2)C(C)(C)C)cc1]

### Step 1: Preparation of 2-(2-(2-((1-(tert-butoxycarbonyl)-3-(pyridin-2-yl)azetidin-3-yl)amino)-2-oxoethoxy)ethoxy)acetic acid

To a solution of 2,2 [symbol] (ethane-1,2-diylbis(oxy))diacetic acid (0.30 g, 1.7 mmol, 1.0 eq) in DCM (3.0 mL) was added HATU (0.32 g, 0.84 mmol, 0.50 eq) and DIEA (0.43 g, 3.4 mmol, 2.0 eq), then tert-butyl 3-amino-3-(pyridin-2-yl)azetidine-1-carboxylate (0.21 g, 0.84 mol, 0.50 eq) was added, the mixture was stirred at 25° C. for 1 hour to give a yellow solution. The reaction mixture was concentrated under reduced pressure to give a residue. The residue was purified by flash silica gel chromatography to give 2-(2-(2-((1-(tert-butoxycarbonyl)-3-(pyridin-2-yl)azetidin-3-yl)amino)-2-oxoethoxy)ethoxy)acetic acid (0.20 g, 26% yield) as a yellow oil. LCMS (ESI) m/z=410.1. ¹H NMR (400 MHz, METHANOL-d₄) δ 8.57-8.63 (m, 1H) 7.98 (d, J=8.0 Hz, 1H) 7.66 (d, J=8.00 Hz, 1H) 7.41-7.49 (m, 1H) 4.28-4.38 (m, 4H) 4.14 (m, 2H) 4.08 (s, 2H) 3.76 (s, 4H) 1.44 (s, 9H)

### Step 2: Preparation of tert-butyl 3-(2-(2-(2-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-2-oxoethoxy)ethoxy)acetamido)-3-(pyridin-2-yl)azetidine-1-carboxylate

To a solution of 2-(2-(2-((1-(tert-butoxycarbonyl)-3-(pyridin-2-yl)azetidin-3-yl)amino)-2-oxoethoxy)ethoxy)acetic acid (0.20 g, 0.49 mmol, 1.0 eq) in DCM (5.0 mL) was added HATU (0.23 g, 0.59 mmol, 1.2 eq) and DIEA (0.13 g, 0.98 mmol, 2.0 eq), then (2S,4R)-1-((S)-2-amino-3,3-dimethylbutanoyl)-4-hydroxy-N—((S)-1-(4-(4-methylthiazol-5-yl)phenyl)ethyl)pyrrolidine-2-carboxamide (0.22 g, 0.49 mmol, 1.0 eq) was added. The mixture was stirred at 25° C. for 1 hour to give a yellow solution. The reaction mixture was concentrated under reduced pressure to give a residue. The residue was purified by reversed phase (column:300 g; mobile phase: [water (TFA)-ACN]; B %: 0%-30%, 30 min). The solution was lyophilization to give tert-butyl 3-(2-(2-(2-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-2-oxoethoxy)ethoxy)acetamido)-3-(pyridin-2-yl)azetidine-1-carboxylate (0.16 g, 40% yield) as a white oil. LCMS (ESI) m/z=836.4. ¹H NMR (400 MHz, METHANOL-d₄) δ 9.03 (s, 1H) 8.61-8.66 (m, 1H) 7.95-8.05 (m, 1H) 7.64-7.74 (m, 1H) 7.43 (m, 5H) 4.70 (s, 1H) 4.55-4.60 (m, 1H) 4.36-4.45 (m, 5H) 4.11-4.16 (m, 3H) 4.09 (s, 1H) 3.98 (s, 1H) 3.82 (s, 5H) 3.75 (m, 1H) 2.49 (s, 3H) 2.15-2.25 (m, 1H) 1.89-2.00 (m, 1H) 1.48 (s, 9H) 1.47 (s, 3H) 1.05 (s, 9H)

### Step 3: Preparation of (2S,4R)-1-((S)-3,3-dimethyl-2-(2-(2-(2-oxo-2-((3-(pyridin-2-yl)azetidin-3-yl)amino)ethoxy)ethoxy)acetamido)butanoyl)-4-hydroxy-N—((S)-1-(4-(4-methylthiazol-5-yl)phenyl)ethyl)pyrrolidine-2-carboxamide

To a solution of tert-butyl 3-(2-(2-(2-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-2-oxoethoxy)ethoxy)acetamido)-3-(pyridin-2-yl)azetidine-1-carboxylate (0.15 g, 0.16 mmol, 1.0 eq) in DCM (1.0 mL) was added TFA (0.30 mL). Then the mixture was stirred at 25° C. for 1 hour to give a yellow solution. The reaction mixture was concentrated under reduced pressure to give (2S,4R)-1-((S)-3,3-dimethyl-2-(2-(2-(2-oxo-2-((3-(pyridin-2-yl)azetidin-3-yl)amino)ethoxy)ethoxy)acetamido)butanoyl)-4-hydroxy-N—((S)-1-(4-(4-methylthiazol-5-yl)phenyl)ethyl)pyrrolidine-2-carboxamide (0.15 g, crude) as a yellow oil.

LCMS (ESI) m/z=736.4.

### Step 4: Preparation of tert-butyl ((3S,6S,10aR)-3-(3-(2-(2-(2-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-2-oxoethoxy)ethoxy)acetamido)-3-(pyridin-2-yl)azetidine-1-carbonyl)-5-oxodecahydropyrrolo[1,2-a]azocin-6-yl)carbamate

To a solution of (3S,6S,10aR)-6-((tert-butoxycarbonyl)amino)-5-oxodecahydropyrrolo[1,2-a]azocine-3-carboxylic acid (50 mg, 0.15 mmol, 1.0 eq) in DCM (1.0 mL) was added HATU (70 mg, 0.18 mmol, 1.2 eq), a solution of (2S,4R)-1-((S)-3,3-dimethyl-2-(2-(2-(2-oxo-2-((3-(pyridin-2-yl)azetidin-3-yl)amino)ethoxy)ethoxy)acetamido)butanoyl)-4-hydroxy-N—((S)-1-(4-(4-methylthiazol-5-yl)phenyl)ethyl)pyrrolidine-2-carboxamide (0.11 g, 0.15 mmol, 1.0 eq), DIEA (99 mg, 0.77 mmol, 5.0 eq) in DCM (1.0 mL) was added to the mixture. Then the mixture was stirred at 25° C. for 1 hour to give a yellow solution. The reaction mixture was concentrated under reduced pressure to give a residue. The residue was purified by flash silica gel chromatography to give tert-butyl ((3S,6S,10aR)-3-(3-(2-(2-(2-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-2-oxoethoxy)ethoxy)acetamido)-3-(pyridin-2-yl)azetidine-1-carbonyl)-5-oxodecahydropyrrolo[1,2-a]azocin-6-yl)carbamate (50 mg, 31% yield) as a white oil. LCMS (ESI) m/z=522.9.

### Step 5: Preparation of (2S,4R)-1-((S)-2-(2-(2-(2-((1-((3S,6S,10aR)-6-amino-5-oxodecahydropyrrolo[1,2-a]azocine-3-carbonyl)-3-(pyridin-2-yl)azetidin-3-yl)amino)-2-oxoethoxy)ethoxy)acetamido)-3,3-dimethylbutanoyl)-4-hydroxy-N—((S)-1-(4-(4-methylthiazol-5-yl)phenyl)ethyl)pyrrolidine-2-carboxamide

To a solution of tert-butyl ((3 S,6S,10aR)-3-(3-(2-(2-(2-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-2-oxoethoxy)ethoxy)acetamido)-3-(pyridin-2-yl)azetidine-1-carbonyl)-5-oxodecahydropyrrolo[1,2-a]azocin-6-yl)carbamate (50 mg, 48 μmol, 1.0 eq) in DCM (1.0 mL) was added TFA (1.0 mL). Then the mixture was stirred at 25° C. for 1 hour to give yellow solution. The reaction mixture was concentrated under reduced pressure to give (2S,4R)-1-((S)-2-(2-(2-(2-((1-((3S,6S,10aR)-6-amino-5-oxodecahydropyrrolo[1,2-a]azocine-3-carbonyl)-3-(pyridin-2-yl)azetidin-3-yl)amino)-2-oxoethoxy)ethoxy)acetamido)-3,3-dimethylbutanoyl)-4-hydroxy-N—((S)-1-(4-(4-methylthiazol-5-yl)phenyl)ethyl)pyrrolidine-2-carboxamide (50.0 mg, crude) as a yellow oil. LCMS (ESI) m/z=944.6.

### Step 6: Preparation of (difluoro(2-(((3S,6S,10aR)-3-(3-(2-(2-(2-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-2-oxoethoxy)ethoxy)acetamido)-3-(pyridin-2-yl)azetidine-1-carbonyl)-5-oxodecahydropyrrolo[1,2-a]azocin-6-yl)carbamoyl)benzo[b]thiophen-5-yl)methyl)phosphonic acid

To a solution of (difluoro(2-((4-nitrophenoxy)carbonyl)benzo[b]thiophen-5-yl)methyl)phosphonic acid (20 mg, 46 μmol, 1.0 eq) in DCM (1.0 mL) was added HOBt (8.5 mg, 55 μmol, 1.2 eq), Then a solution of (2S,4R)-1-((S)-2-(2-(2-(2-((1-((3S,6S,10aR)-6-amino-5-oxodecahydropyrrolo[1,2-a]azocine-3-carbonyl)-3-(pyridin-2-yl)azetidin-3-yl)amino)-2-oxoethoxy)ethoxy)acetamido)-3,3-dimethylbutanoyl)-4-hydroxy-N—((S)-1-(4-(4-methylthiazol-5-yl)phenyl)ethyl)pyrrolidine-2-carboxamide (44 mg, 47 μmol, 1.0 eq), DIEA (30 mg, 0.23 mmol, 5.0 eq) in DCM (1.0 mL) was added to the mixture. The reaction solution was stirred at 25° C. for 1 hour to give a yellow solution. The reaction mixture was concentrated under reduced pressure to give a residue. The residue was purified by prep-HPLC (column:Welch Xtimate C18 150×25 mm×5 um; mobile phase: water (NH₃H₂O)-ACN; B %: 5%-35%, 8 min). The solution was lyophilization to give (difluoro(2-(((3S,6S,10aR)-3-(3-(2-(2-(2-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-2-oxoethoxy)ethoxy)acetamido)-3-(pyridin-2-yl)azetidine-1-carbonyl)-5-oxodecahydropyrrolo[1,2-a]azocin-6-yl)carbamoyl)benzo[b]thiophen-5-yl)methyl)phosphonic acid (Compound 31, 4.6 mg, 8.0% yield) as a white solid. LCMS (ESI) m/z=617.9. ¹H NMR (400 MHz, METHANOL-d₄) δ 8.87-8.93 (m, 1H) 8.53 (m, 1H) 8.07-8.20 (m, 2H) 7.90 (m, 1H) 7.76-7.83 (m, 1H) 7.66-7.75 (m, 1H) 7.46-7.52 (m, 1H) 7.32-7.43 (m, 5H) 5.00-5.06 (m, 1H) 4.69 (s, 2H) 4.47-4.60 (m, 4H) 4.42 (s, 3H) 4.03-4.16 (m, 4H) 3.67-3.85 (m, 7H) 2.41-2.49 (m, 3H) 2.16-2.38 (m, 4H) 1.91-2.11 (m, 8H) 1.63 1.74 (m, 2H) 1.36-1.38 (m, 3H) 1.02 (s, 9H).

### Preparation of ((2-(((3S,6S,10aR)-3-(3-(2-(2-(2-(2-((2-(2,6-dioxopiperidin-3-yl)-1,3-dioxoisoindolin-4-yl)oxy)acetamido)ethoxy)ethoxy)acetamido)-3-(pyridin-2-yl)azetidine-1-carbonyl)-5-oxodecahydropyrrolo[1,2-a]azocin-6-yl)carbamoyl)benzo[b]thiophen-5-yl)difluoromethyl)phosphonic acid (Compound 32)

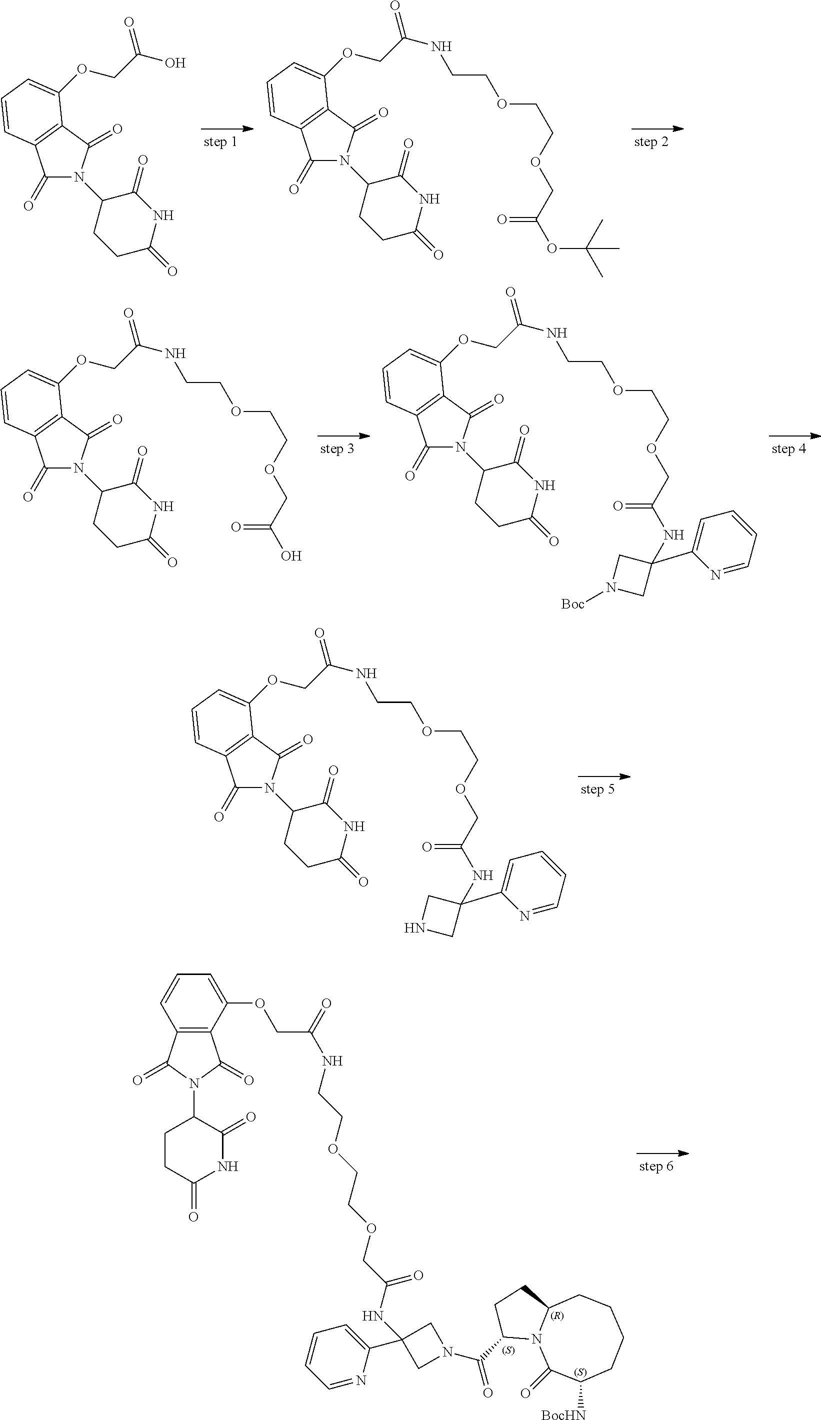

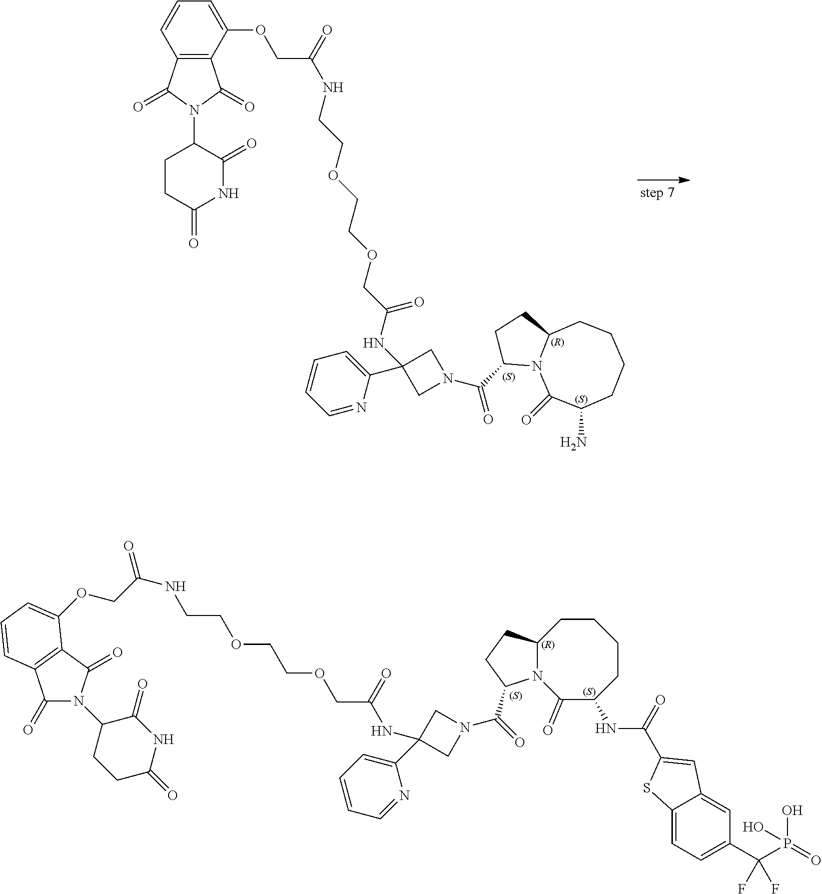

### Step 1: Preparation of tert-butyl 2-(2-(2-(2-((2-(2,6-dioxopiperidin-3-yl)-1,3-dioxoisoindolin-4-yl)oxy)acetamido)ethoxy)ethoxy)acetate

To a solution of 2-{[2-(2,6-dioxopiperidin-3-yl)-1,3-dioxo-2,3-dihydro-1H-isoindol-4-yl]oxy}acetic acid (0.3 g, 0.90 mmol), HATU (0.52 g, 1.4 mmol) in DMF (5 mL) was added ethylbis(propan-2-yl)amine (0.35 g, 2.7 mmol), then tert-butyl 2-[2-(2-aminoethoxy)ethoxy]acetate (0.24 g, 1.1 mmol) was added in it and stirred at 25° C. for 2 hours to give a yellow solution. The reaction mixture was diluted with water (20 mL) and extracted with ethyl acetate (20 mL×2), the combined organic layers were washed with saturated brine (20 mL×2), dried over Na₂SO₄, filtered and concentrated under reduced pressure to give a tert-butyl 2-(2-(2-(2-((2-(2,6-dioxopiperidin-3-yl)-1,3-dioxoisoindolin-4-yl)oxy)acetamido)ethoxy)ethoxy)acetate (0.60 g, crude) as a brown oil. LCMS (ESI) m/z=534.1.

### Step 2: Preparation of 2-(2-(2-(2-((2-(2,6-dioxopiperidin-3-yl)-1,3-dioxoisoindolin-4-yl)oxy)acetamido)ethoxy)ethoxy)acetic acid

To a solution of tert-butyl 2-(2-(2-(2-((2-(2,6-dioxopiperidin-3-yl)-1,3-dioxoisoindolin-4-yl)oxy)acetamido)ethoxy)ethoxy)acetate (0.15 g, 0.28 mmol) in DCM (1.5 mL) was added TFA (1.5 mL) and stirred at 25° C. for 1 hour to give a yellow solution. The reaction mixture was concentrated under reduced pressure to give 2-(2-(2-(2-((2-(2,6-dioxopiperidin-3-yl)-1,3-dioxoisoindolin-4-yl)oxy)acetamido)ethoxy)ethoxy)acetic acid (0.13 g, crude) as a yellow oil. LCMS (ESI) m/z=478.2.

### Step 3: Preparation of tert-butyl 3-(2-(2-(2-(2-((2-(2,6-dioxopiperidin-3-yl)-1,3-dioxoisoindolin-4-yl)oxy)acetamido)ethoxy)ethoxy)acetamido)-3-(pyridin-2-yl)azetidine-1-carboxylate

To a solution of 2-(2-(2-(2-((2-(2,6-dioxopiperidin-3-yl)-1,3-dioxoisoindolin-4-yl)oxy)acetamido)ethoxy)ethoxy)acetic acid (0.13 g, 0.27 mmol) and HATU (0.16 g, 0.41 mmol) in DMF (3 mL) was added ethylbis(propan-2-yl)amine (0.14 g, 1.1 mmol), then tert-butyl 3-amino-3-(pyridin-2-yl)azetidine-1-carboxylate (81 mg, 0.33 mmol) was added in it and stirred at 25° C. for 2 hours to give a yellow solution. The reaction mixture was filtered and the filter was purified by reverse phase (0.1% FA) to give tert-butyl 3-(2-(2-(2-(2-((2-(2,6-dioxopiperidin-3-yl)-1,3-dioxoisoindolin-4-yl)oxy)acetamido)ethoxy)ethoxy)acetamido)-3-(pyridin-2-yl)azetidine-1-carboxylate (80 mg, 42% yield) as a yellow solid. LCMS (ESI) m/z=709.3.

### Step 4: Preparation of 2-((2-(2,6-dioxopiperidin-3-yl)-1,3-dioxoisoindolin-4-yl)oxy)-N-(2-(2-(2-oxo-2-((3-(pyridin-2-yl)azetidin-3-yl)amino)ethoxy)ethoxy)ethyl)acetamide

To a solution of tert-butyl 3-(2-(2-(2-(2-((2-(2,6-dioxopiperidin-3-yl)-1,3-dioxoisoindolin-4-yl)oxy)acetamido)ethoxy)ethoxy)acetamido)-3-(pyridin-2-yl)azetidine-1-carboxylate (40 mg, 56 μmol) in DCM (0.5 mL) was added TFA (0.5 mL) and stirred at 25° C. for 1 hour to give a yellow solution. The reaction mixture was concentrated under reduced pressure to give 2-((2-(2,6-dioxopiperidin-3-yl)-1,3-dioxoisoindolin-4-yl)oxy)-N-(2-(2-(2-oxo-2-((3-(pyridin-2-yl)azetidin-3-yl)amino)ethoxy)ethoxy)ethyl)acetamide (40 mg, crude) as a yellow oil. LCMS (ESI) m/z=609.1.

### Step 5: Preparation of tert-butyl ((3S,6S,10aR)-3-(3-(2-(2-(2-(2-((2-(2,6-dioxopiperidin-3-yl)-1,3-dioxoisoindolin-4-yl)oxy)acetamido)ethoxy)ethoxy)acetamido)-3-(pyridin-2-yl)azetidine-1-carbonyl)-5-oxodecahydropyrrolo[1,2-a]azocin-6-yl)carbamate

To a solution of (3S,6S,10aR)-6-((tert-butoxycarbonyl)amino)-5-oxodecahydropyrrolo[1,2-a]azocine-3-carboxylic acid (20 mg, 61 μmol) in DMF (1 mL) was added HATU (35 mg, 92 μmol) and ethylbis(propan-2-yl)amine (79 mg, 0.61 mmol). The mixture was stirred at 25° C. for 10 minutes. Then, a solution of 2-{[2-(2,6-dioxopiperidin-3-yl)-1,3-dioxo-2,3-dihydro-1H-isoindol-4-yl]oxy}-N-{2-[2-({[3-(pyridin-2-yl)azetidin-3-yl]carbamoyl}methoxy)ethoxy]ethyl}acetamide (45 mg, 73 μmol), ethylbis(propan-2-yl)amine (79 mg, 0.61 mmol) in DMF (1 mL) was added to the mixture. The mixture was stirred at 25° C. for 2 hours to give a yellow solution. The reaction mixture was filtered and the filtrate was purified by reverse phase chromatography (0.1% FA) and concentrated to give tert-butyl ((3S,6S,10aR)-3-(3-(2-(2-(2-(2-((2-(2,6-dioxopiperidin-3-yl)-1,3-dioxoisoindolin-4-yl)oxy)acetamido)ethoxy)ethoxy)acetamido)-3-(pyridin-2-yl)azetidine-1-carbonyl)-5-oxodecahydropyrrolo[1,2-a]azocin-6-yl)carbamate (30 mg, 53% yield) as a yellow oil. LCMS (ESI) m/z=917.3.

### Step 6: Preparation of N-(1-((3S,6S,10aR)-6-amino-5-oxodecahydropyrrolo[1,2-a]azocine-3-carbonyl)-3-(pyridin-2-yl)azetidin-3-yl)-2-(2-(2-(2-((2-(2,6-dioxopiperidin-3-yl)-1,3-dioxoisoindolin-4-yl)oxy)acetamido)ethoxy)ethoxy)acetamide

To a solution tert-butyl ((3S,6S,10aR)-3-(3-(2-(2-(2-(2-((2-(2,6-dioxopiperidin-3-yl)-1,3-dioxoisoindolin-4-yl)oxy)acetamido)ethoxy)ethoxy)acetamido)-3-(pyridin-2-yl)azetidine-1-carbonyl)-5-oxodecahydropyrrolo[1,2-a]azocin-6-yl)carbamate (30 mg, 33 μmol) in DCM (0.5 mL) and TFA (0.5 mL), the mixture was stirred at 25° C. for 1 hour to give a yellow solution. The reaction mixture was concentrated under reduced pressure to give N-(1-((3S,6S,10aR)-6-amino-5-oxodecahydropyrrolo[1,2-a]azocine-3-carbonyl)-3-(pyridin-2-yl)azetidin-3-yl)-2-(2-(2-(2-((2-(2,6-dioxopiperidin-3-yl)-1,3-dioxoisoindolin-4-yl)oxy)acetamido)ethoxy)ethoxy)acetamide (30 mg, crude) as a yellow oil. LCMS (ESI) m/z=817.1.

### Step 7: Preparation of ((2-(((3S,6S,10aR)-3-(3-(2-(2-(2-(2-((2-(2,6-dioxopiperidin-3-yl)-1,3-dioxoisoindolin-4-yl)oxy)acetamido)ethoxy)ethoxy)acetamido)-3-(pyridin-2-yl)azetidine-1-carbonyl)-5-oxodecahydropyrrolo[1,2-a]azocin-6-yl)carbamoyl)benzo[b]thiophen-5-yl)difluoromethyl)phosphonic acid

To a solution of [difluoro({2-[(4-nitrophenoxy)carbonyl]-1-benzothiophen-5-yl})methyl]phosphonic acid (12 mg, 28 μmol) in DCM (1 mL) was added HOBt (5.1 mg, 33 μmol) and triethylamine (11 mg, 0.11 mmol), then a solution of N-(1-((3S,6S,10aR)-6-amino-5-oxodecahydropyrrolo[1,2-a]azocine-3-carbonyl)-3-(pyridin-2-yl)azetidin-3-yl)-2-(2-(2-(2-((2-(2,6-dioxopiperidin-3-yl)-1,3-dioxoisoindolin-4-yl)oxy)acetamido)ethoxy)ethoxy)acetamide (27 mg, 33 μmol) and triethylamine (11 mg, 0.11 mmol) in DCM (1 mL) was added in it and stirred at 25° C. for 15 minutes to give a yellow solution. The reaction mixture was concentrated under reduced pressure to give a residue and was purified by prep-HPLC (column: Welch Ultimate C18 150×25 mm×5 um; mobile phase: water (TFA)-ACN]; B %: 15%-45%, 10 min). The solution was lyophilized to give ((2-(((3S,6S,10aR)-3-(3-(2-(2-(2-(2-((2-(2,6-dioxopiperidin-3-yl)-1,3-dioxoisoindolin-4-yl)oxy)acetamido)ethoxy)ethoxy)acetamido)-3-(pyridin-2-yl)azetidine-1-carbonyl)-5-oxodecahydropyrrolo[1,2-a]azocin-6-yl)carbamoyl)benzo[b]thiophen-5-yl)difluoromethyl)phosphonic acid (Compound 32, 10 mg, 32% yield) as a white solid.

LCMS (ESI) m/z=1107.3. ¹H NMR (400 MHz, DMSO-d₆) δ 9.00-8.92 (m, 1H), 8.88 (d, J=6.4 Hz, 1H), 8.62-8.55 (m, 1H), 8.32 (s, 1H), 8.14 (d, J=8.4 Hz, 1H), 8.07 (s, 1H), 8.04-7.98 (m, 1H), 7.82-7.74 (m, 2H), 7.58 (d, J=8.8 Hz, 1H), 7.50-7.46 (m, 1H), 7.41-7.33 (m, 2H), 7.32-7.27 (m, 1H), 5.16-5.05 (m, 1H), 4.98-4.90 (m, 1H), 4.76 (d, J=9.2 Hz, 2H), 4.63 (d, J=8.0 Hz, 1H), 4.53-4.48 (m, 1H), 4.40-4.24 (m, 4H), 4.18 (s, 2H), 4.02-3.97 (m, 3H), 3.65 (d, J=3.6 Hz, 2H), 3.59-3.56 (m, 2H), 3.47-3.44 (m, 1H), 3.31-3.29 (m, 2H), 2.21-2.15 (m, 1H), 2.10-1.97 (m, 3H), 1.91-1.78 (m, 6H), 1.69-1.47 (m, 4H).

### Preparation of (difluoro(2-(((3S,6S,10aR)-3-(3-(3-(2-(2-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-2-oxoethoxy)ethoxy)propanamido)-3-(pyridin-2-yl)azetidine-1-carbonyl)-5-oxodecahydropyrrolo[1,2-a]azocin-6-yl)carbamoyl)benzo[b]thiophen-5-yl)methyl)phosphonic acid (Compound 33)

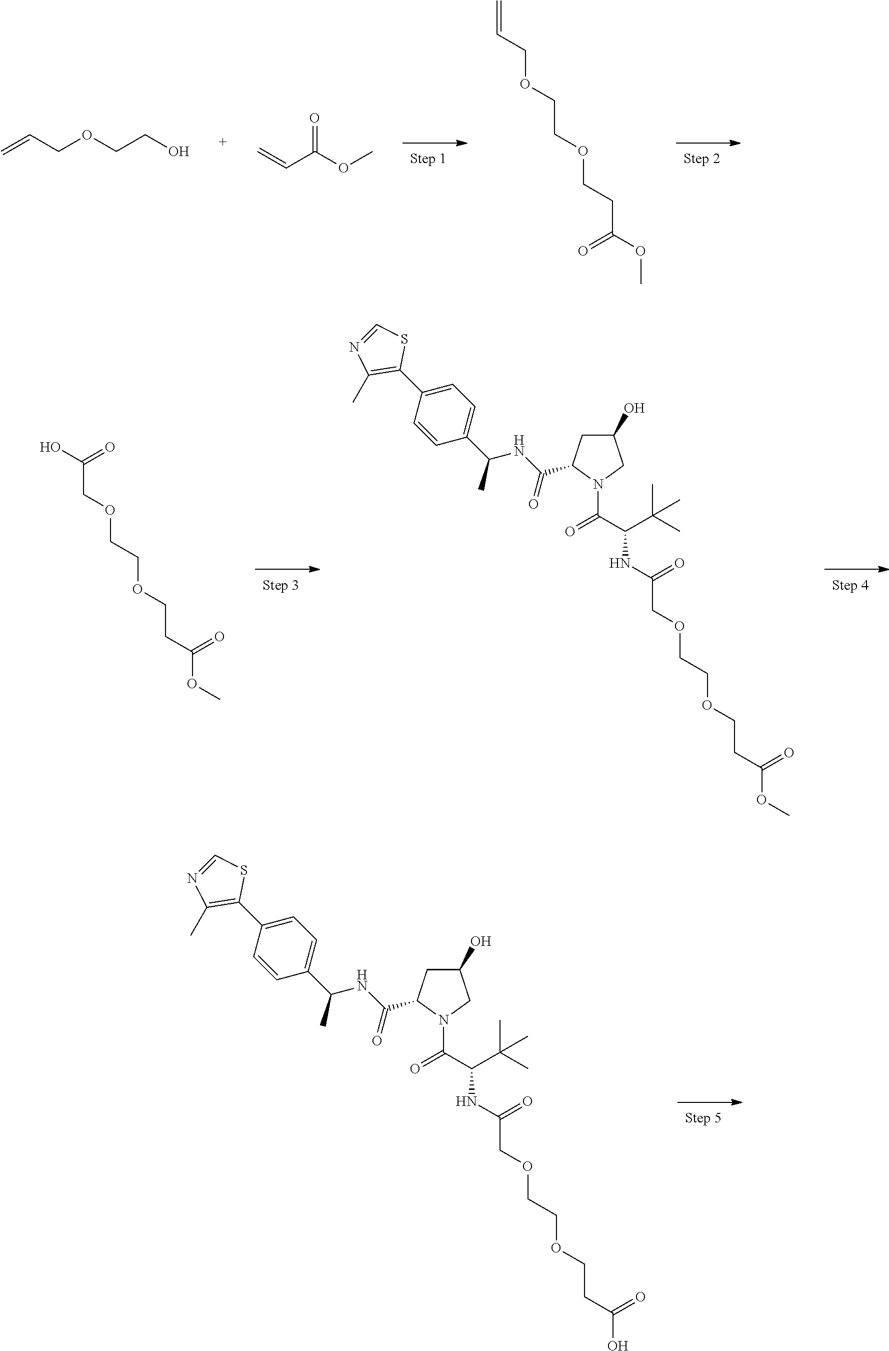

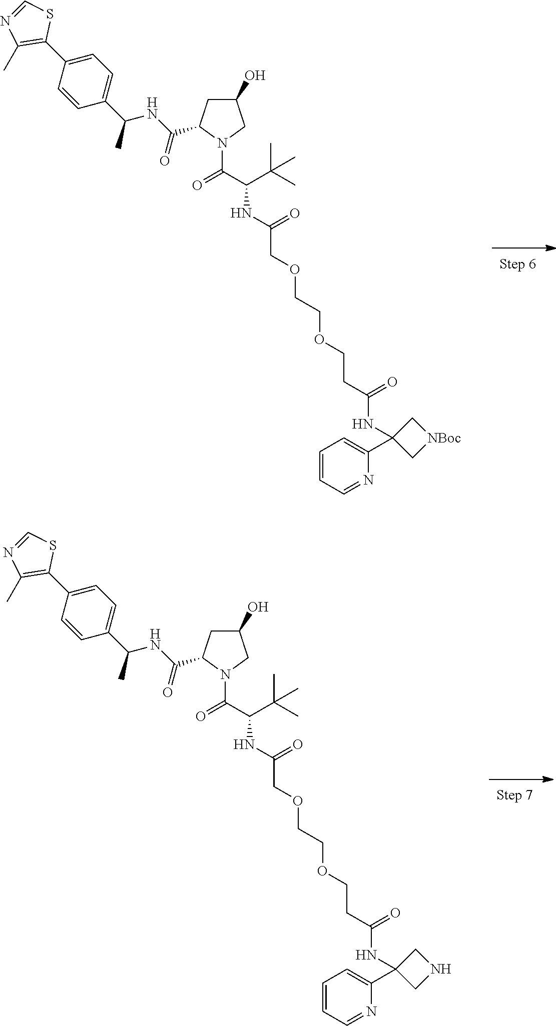 [SMILES: Cc1ncsc1-c1ccc([C@H](C)NC(=O)[C@@H]2C[C@@H](O)CN2C(=O)[C@@H](NC(=O)COCCOCCC(=O)NC2(c3ccccn3)CN(C(=O)OC(C)(C)C)C2)C(C)(C)C)cc1 ; Cc1ncsc1-c1ccc([C@H](C)NC(=O)[C@@H]2C[C@@H](O)CN2C(=O)[C@@H](NC(=O)COCCOCCC(=O)NC2(c3ccccn3)CNC2)C(C)(C)C)cc1]

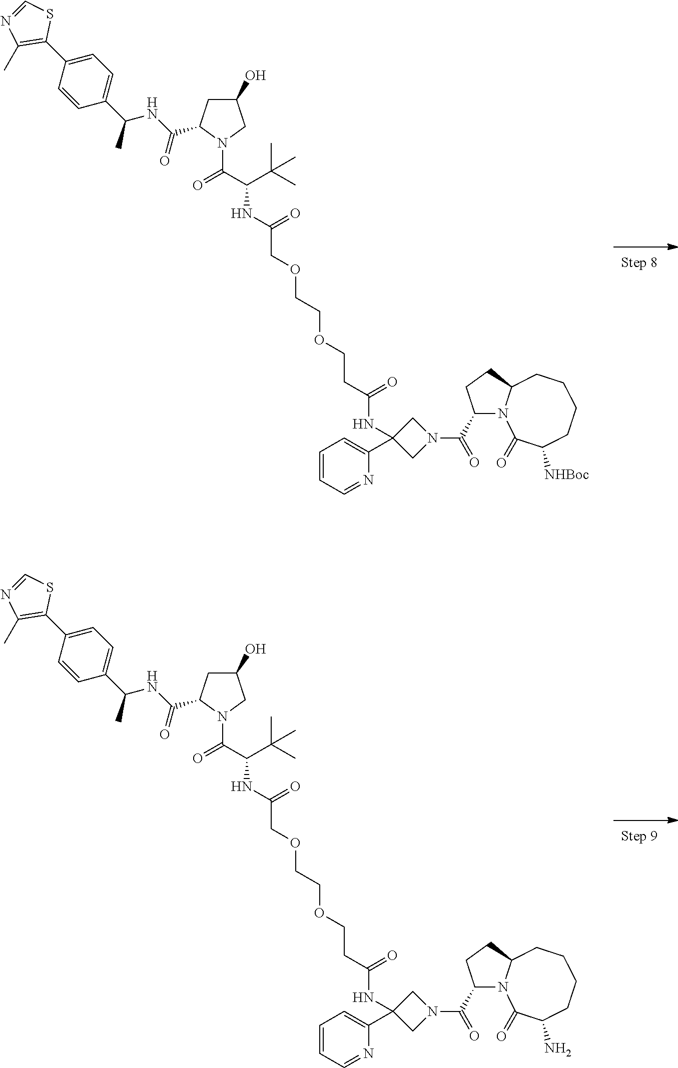 [SMILES: Cc1ncsc1-c1ccc([C@H](C)NC(=O)[C@@H]2C[C@@H](O)CN2C(=O)[C@@H](NC(=O)COCCOCCC(=O)NC2(c3ccccn3)CN(C(=O)[C@@H]3CC[C@H]4CCCC[C@H](NC(=O)OC(C)(C)C)C(=O)N43)C2)C(C)(C)C)cc1 ; Cc1ncsc1-c1ccc([C@H](C)NC(=O)[C@@H]2C[C@@H](O)CN2C(=O)[C@@H](NC(=O)COCCOCCC(=O)NC2(c3ccccn3)CN(C(=O)[C@@H]3CC[C@H]4CCCC[C@H](N)C(=O)N43)C2)C(C)(C)C)cc1]

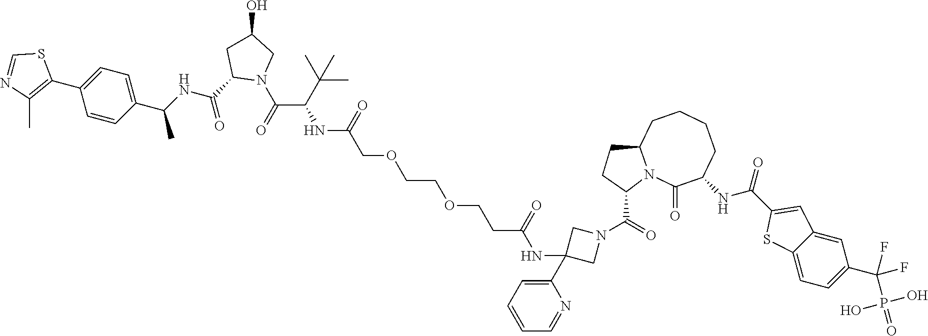 [SMILES: Cc1ncsc1-c1ccc([C@H](C)NC(=O)[C@@H]2C[C@@H](O)CN2C(=O)[C@@H](NC(=O)COCCOCCC(=O)NC2(c3ccccn3)CN(C(=O)[C@@H]3CC[C@H]4CCCC[C@H](NC(=O)c5cc6cc(C(F)(F)P(=O)(O)O)ccc6s5)C(=O)N43)C2)C(C)(C)C)cc1]

### Step 1: Preparation of methyl 3-(2-(allyloxy)ethoxy)propanoate

To a solution of 2-(prop-2-en-1-yloxy)ethan-1-ol (10 g, 98 mmol, 1 eq) in tetrahydrofuran (200 mL) was added sodium (1.1 g, 49 mmol, 0.50 eq) at 0° C. The mixture was stirred at 60° C. for 3 hours to give a yellow solution. The mixture was filtered and the filtrate was directly used next step without work up. To a solution of methyl acrylate (8.4 g, 98 mmol, 1 eq) in tetrahydrofuran (100 mL) was added sodium 2-(allyloxy)ethan-1-olate solution (200 mL, 49 mmol, 0.5 eq) at 0° C. The mixture was stirred at 25° C. for 12 hours to give a yellow solution. The reaction was diluted with water (50.0 mL) and extracted with ethyl acetate (50.0 mL×2), the combined organic layers were washed with saturated brine (40.0 mL×2), dried over Na₂SO₄, filtered and concentrated under reduced pressure to give crude product. The residue was purified by column chromatography to give methyl 3-(2-(allyloxy)ethoxy)propanoate (1.0 g, 5.3 mmol, 5.4% yield) as yellow oil.

### Step 2: Preparation of 2-(2-(3-methoxy-3-oxopropoxy)ethoxy)acetic acid

To a solution of methyl 3-(2-(allyloxy)ethoxy)propanoate (1.0 g, 5.3 mmol, 1 eq) in water (4.0 mL), ACN (4.0 mL) and CHCl₃ (6.0 mL) was added NaIO₄ (5.7 g, 27 mmol, 5 eq) and RuCl₃ (549 mg, 2.7 mmol, 0.5 eq). The reaction was stirred at 25° C. for 1 hour to give a green-black muddy solution. The reaction was diluted with water (50 mL) and extracted with ethyl acetate (50 mL×2), the combined organic layers were washed with saturated brine (40 mL×2), dried over Na₂SO₄, filtered and concentrated under reduced pressure to give 2-(2-(3-methoxy-3-oxopropoxy)ethoxy)acetic acid (1.2 g, crude) was obtained as green-black solid and was used into the next step without further purification. ¹H NMR (400 MHz, CHLOROFORM-d) δ 4.36-4.29 (m, 1H), 4.19-4.15 (m, 2H), 3.84-3.80 (m, 2H), 3.73-3.71 (m, 3H), 3.69-3.66 (m, 2H), 3.35 (d, J=13.6 Hz, 2H), 2.64 (t, J=6.4 Hz, 2H).

### Step 3: Preparation of methyl 3-(2-(2-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-2-oxoethoxy)ethoxy)propanoate

To a solution of 2-(2-(3-methoxy-3-oxopropoxy)ethoxy)acetic acid (1.2 g, 5.8 mmol, 1 eq) in DMF (5.0 mL) was added HATU (2.4 g, 6.4 mmol, 1.1 eq) and DIEA (2.2 g, 17 mmol, 3 eq) the reaction was stirred 10 minutes to give yellow clean solution, Then was added a solution of (2S,4R)-1-((S)-2-amino-3,3-dimethylbutanoyl)-4-hydroxy-N—((S)-1-(4-(4-methylthiazol-5-yl)phenyl)ethyl)pyrrolidine-2-carboxamide (2.6 g, 5.8 mmol, 1 eq) in DMF (5.0 mL) which was added DIEA (2.2 g, 17 mmol, 3 eq) adjust to pH=8~9 and then was stirred at 25° C. for 30 minutes to give a yellow clean solution. The reaction was diluted with water (50 mL) and extracted with ethyl acetate (50 mL×2), the combined organic layers were washed with saturated brine (40 mL×2), dried over Na₂SO₄, filtered and concentrated under reduced pressure to give a residue. The residue was purified by reversed phase prep HPLC (TFA modifier) and combined fractions were lyophilized to afford methyl 3-(2-(2-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-2-oxoethoxy)ethoxy)propanoate (1.0 g, 70% purity, 1.1 mmol, 19.0% yield) was obtained as a brassy yellow solid. LCMS (ESI) m/z=633.1

### Step 4: Preparation of 3-(2-(2-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-2-oxoethoxy)ethoxy)propanoic acid

To a solution of methyl 3-(2-(2-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-2-oxoethoxy)ethoxy)propanoate (1.0 g, 70% purity, 1.1 mmol, 1 eq) in THF (3 mL), H₂O (3 mL) and MeOH (3 mL) was added lithium hydroxide (46 mg, 1.1 mmol, 1.0 eq), The mixture was stirred at 25° C. for 16 hours to give a light-yellow clean solution. The reaction was purified by reversed phase (TFA) then lyophilization to give 3-(2-(2-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-2-oxoethoxy)ethoxy)propanoic acid (200 mg, 323 mol, 20% yield) as a white solid. LCMS (ESI) m/z=619.2. ¹H NMR (400 MHz, METHANOL-d₄) δ 9.00 (s, 1H), 7.49-7.39 (m, 4H), 5.01 (q, J=6.8 Hz, 1H), 4.71-4.66 (m, 1H), 4.62-4.54 (m, 1H), 4.47-4.41 (m, 1H), 4.09-4.01 (m, 2H), 3.89-3.66 (m, 8H), 2.60 (t, J=6.4 Hz, 2H), 2.50 (s, 3H), 2.24-2.16 (m, 1H), 1.97 (dt, J=4.4, 8.8 Hz, 1H), 1.59-1.48 (m, 3H), 1.10-1.01 (m, 9H)

### Step 5: Preparation of tert-butyl 3-(3-(2-(2-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-2-oxoethoxy)ethoxy)propanamido)-3-(pyridin-2-yl)azetidine-1-carboxylate

To a solution of 3-(2-(2-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-2-oxoethoxy)ethoxy)propanoic acid (180 mg, 290 mol, 1 eq), EDCI (83 mg, 435 mol, 1.5 eq) and tert-butyl 3-amino-3-(pyridin-2-yl)azetidine-1-carboxylate (72.3 mg, 290 mol, 1 eq) in pyridine (4 mL) was s stirred at 25° C. for 1 hours to give a yellow clean solution. The reaction mixture was concentrated under reduced pressure to give a residue. The residue was purified by reversed phase (TFA) then lyophilization to give tert-butyl 3-(3-(2-(2-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-2-oxoethoxy)ethoxy)propanamido)-3-(pyridin-2-yl)azetidine-1-carboxylate (140 mg, 164 mol, 57% yield) as a white solid. LCMS (ESI) m/z=850.4

### Step 6: Preparation of (2S,4R)-1-((S)-3,3-dimethyl-2-(2-(2-(3-oxo-3-((3-(pyridin-2-yl)azetidin-3-yl)amino)propoxy)ethoxy)acetamido)butanoyl)-4-hydroxy-N—((S)-1-(4-(4-methylthiazol-5-yl)phenyl)ethyl)pyrrolidine-2-carboxamide

To a solution of tert-butyl 3-(3-(2-(2-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-2-oxoethoxy)ethoxy)propanamido)-3-(pyridin-2-yl)azetidine-1-carboxylate (140 mg, 164 mol, 1 eq) in DCM (3.0 mL) was added TFA (1.0 mL). The mixture was stirred at 20° C. for 1 hour to give a yellow clear solution. The reaction mixture was concentrated under reduced pressure to give (2S,4R)-1-((S)-3,3-dimethyl-2-(2-(2-(3-oxo-3-((3-(pyridin-2-yl)azetidin-3-yl)amino)propoxy)ethoxy)acetamido)butanoyl)-4-hydroxy-N—((S)-1-(4-(4-methylthiazol-5-yl)phenyl)ethyl)pyrrolidine-2-carboxamide (140 mg, crude) as a yellow oil. LCMS (ESI) m/z=750.4

### Step 7: Preparation of tert-butyl ((3S,6S,10aR)-3-(3-(3-(2-(2-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-2-oxoethoxy)ethoxy)propanamido)-3-(pyridin-2-yl)azetidine-1-carbonyl)-5-oxodecahydropyrrolo[1,2-a]azocin-6-yl)carbamate

To a solution of (3S,6S,10aR)-6-((tert-butoxycarbonyl)amino)-5-oxodecahydropyrrolo[1,2-a]azocine-3-carboxylic acid (61 mg, 186 mol, 1.0 eq) in DMF (2.0 mL) was added DIEA (72.1 mg, 558 mol, 3.0 eq) and HATU (70.7 mg, 186 mol, 1.0 eq), the mixture was stirred at 25° C. for 10 minutes to give a brownness clean solution then was added a solution of (2S,4R)-1-((S)-3,3-dimethyl-2-(2-(2-(3-oxo-3-((3-(pyridin-2-yl)azetidin-3-yl)amino)propoxy)ethoxy)acetamido)butanoyl)-4-hydroxy-N—((S)-1-(4-(4-methylthiazol-5-yl)phenyl)ethyl)pyrrolidine-2-carboxamide (140 mg, 186 mol, 1.0 eq) in DMF (1.0 mL) was added N,N-diisopropylethylamine (72 mg, 558 mol, 3.0 eq). then the mixture was stirred at 25° C. for 1 hour to give a brown clear solution. The reaction was purified by reverse phase prep HPLC (TFA modifier) and combined fractions were frozen and lyophilized to give tert-butyl ((3S,6S,10aR)-3-(3-(3-(2-(2-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-2-oxoethoxy)ethoxy)propanamido)-3-(pyridin-2-yl)azetidine-1-carbonyl)-5-oxodecahydropyrrolo[1,2-a]azocin-6-yl)carbamate (120 mg, 113 mol, 61% yield) as a white solid. LCMS (ESI) m/z=1058.6. ¹H NMR (400 MHz, CHLOROFORM-d) δ 9.19-8.89 (m, 1H), 8.84-8.75 (m, 1H), 8.58 (d, J=5.2 Hz, 1H), 8.24-8.07 (m, 1H), 7.88-7.71 (m, 1H), 7.62-7.40 (m, 2H), 7.37-7.27 (m, 4H), 5.16-5.00 (m, 2H), 4.79 (dd, J=1.6, 9.2 Hz, 1H), 4.74-4.65 (m, 2H), 4.65-4.51 (m, 5H), 4.47-4.32 (m, 5H), 4.29-4.20 (m, 2H), 4.18-4.00 (m, 3H), 3.95-3.87 (m, 2H), 3.77-3.66 (m, 3H), 3.63-3.55 (m, 3H), 3.33 (s, 3H), 2.18-1.83 (m, 8H), 1.70-1.46 (m, 6H), 1.44-1.37 (m, 5H), 1.08-0.95 (m, 9H).

### Step 8: Preparation of (2S,4R)-1-((S)-2-(2-(2-(3-((1-((3S,6S,10aR)-6-amino-5-oxodecahydropyrrolo[1,2-a]azocine-3-carbonyl)-3-(pyridin-2-yl)azetidin-3-yl)amino)-3-oxopropoxy)ethoxy)acetamido)-3,3-dimethylbutanoyl)-4-hydroxy-N—((S)-1-(4-(4-methylthiazol-5-yl)phenyl)ethyl)pyrrolidine-2-carboxamide

To a solution of tert-butyl ((3 S,6S,10aR)-3-(3-(3-(2-(2-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-2-oxoethoxy)ethoxy)propanamido)-3-(pyridin-2-yl)azetidine-1-carbonyl)-5-oxodecahydropyrrolo[1,2-a]azocin-6-yl)carbamate (150 mg, 141 mol, 1 eq) in DCM (1.0 mL) was added TFA (0.3 mL). The mixture was stirred at 20° C. for 1 hour to give a yellow clear solution. The reaction mixture was concentrated under reduced pressure to give (2S,4R)-1-((S)-2-(2-(2-(3-((1-((3S,6S,10aR)-6-amino-5-oxodecahydropyrrolo[1,2-a]azocine-3-carbonyl)-3-(pyridin-2-yl)azetidin-3-yl)amino)-3-oxopropoxy)ethoxy)acetamido)-3,3-dimethylbutanoyl)-4-hydroxy-N—((S)-1-(4-(4-methylthiazol-5-yl)phenyl)ethyl)pyrrolidine-2-carboxamide (100 mg, crude) as a yellow oil.

### Step 9: Preparation of (difluoro(2-(((3S,6S,10aR)-3-(3-(3-(2-(2-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-2-oxoethoxy)ethoxy)propanamido)-3-(pyridin-2-yl)azetidine-1-carbonyl)-5-oxodecahydropyrrolo[1,2-a]azocin-6-yl)carbamoyl)benzo[b]thiophen-5-yl)methyl)phosphonic acid

To a solution of [difluoro({2-[(4-nitrophenoxy)carbonyl]-1-benzothiophen-5-yl})methyl]phosphonic acid (45 mg, 104 mol, 1.0 eq) in DMF (1.0 mL) was added 1-hydroxybenzotriazole (17 mg, 126 mol, 1.2 eq) and DIEA (40.3 mg, 312 mol, 3.0 eq), the mixture was stirred at 25° C. for 10 minutes to give a yellow clean solution then was added a solution of (2S,4R)-1-((S)-2-(2-(2-(3-((1-((3S,6S,10aR)-6-amino-5-oxodecahydropyrrolo[1,2-a]azocine-3-carbonyl)-3-(pyridin-2-yl)azetidin-3-yl)amino)-3-oxopropoxy)ethoxy)acetamido)-3,3-dimethylbutanoyl)-4-hydroxy-N—((S)-1-(4-(4-methylthiazol-5-yl)phenyl)ethyl)pyrrolidine-2-carboxamide (100 mg, 104 mol, 1.0 eq) in DMF (1.5 mL) was added DIEA (40.3 mg, 312 mol, 3.0 eq). The mixture was stirred at 25° C. for 1 hours to give a yellow clear solution. The reaction was purified by prep-HPLC (neutral condition; (column:Phenomenex luna C18 150*25 mm*10 um, mobile phasewater (NH₄HCO₃)-ACN; B %:B %: 16%-36%, 30 min) to freeze-drying to give (difluoro(2-(((3S,6S,10aR)-3-(3-(3-(2-(2-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-2-oxoethoxy)ethoxy)propanamido)-3-(pyridin-2-yl)azetidine-1-carbonyl)-5-oxodecahydropyrrolo[1,2-a]azocin-6-yl)carbamoyl)benzo[b]thiophen-5-yl)methyl)phosphonic acid (Compound 33, 38.3 mg, 30.6 mol, 30% yield) was obtained as a white solid. LCMS (ESI) m/z=1248.2. ¹H NMR (400 MHz, CHLOROFORM-d) δ 8.86 (d, J=4.8 Hz, 1H), 8.66-8.54 (m, 1H), 8.20-8.11 (m, 1H), 8.07-8.00 (m, 1H), 7.94-7.84 (m, 1H), 7.79-7.71 (m, 2H), 7.69-7.59 (m, 1H), 7.45-7.36 (m, 4H), 7.34-7.22 (m, 1H), 5.05-4.97 (m, 2H), 4.80-4.77 (m, 1H), 4.73-4.65 (m, 1H), 4.62-4.43 (m, 4H), 4.42-4.29 (m, 3H), 4.05-3.99 (m, 2H), 3.87-3.61 (m, 8H), 2.80-2.64 (m, 1H), 2.57-2.42 (m, 4H), 2.34-2.17 (m, 3H), 2.07-1.91 (m, 6H), 1.89-1.77 (m, 3H), 1.73-1.60 (m, 2H), 1.55-1.46 (m, 3H), 1.07-0.97 (m, 9H).

### Preparation of ((2-(((3S,6S,9aS)-3-(3-(4-(4-(4-(1-(2,6-dioxopiperidin-3-yl)-3-methyl-2-oxo-2,3-dihydro-1H-benzo[d]imidazol-5-yl)but-3-yn-1-yl)piperidin-1-yl)pyridin-3-yl)azetidine-1-carbonyl)-5-oxooctahydro-1H-pyrrolo[1,2-a]azepin-6-yl)carbamoyl)benzo[b]thiophen-5-yl)difluoromethyl)phosphonic acid (Compound 34)

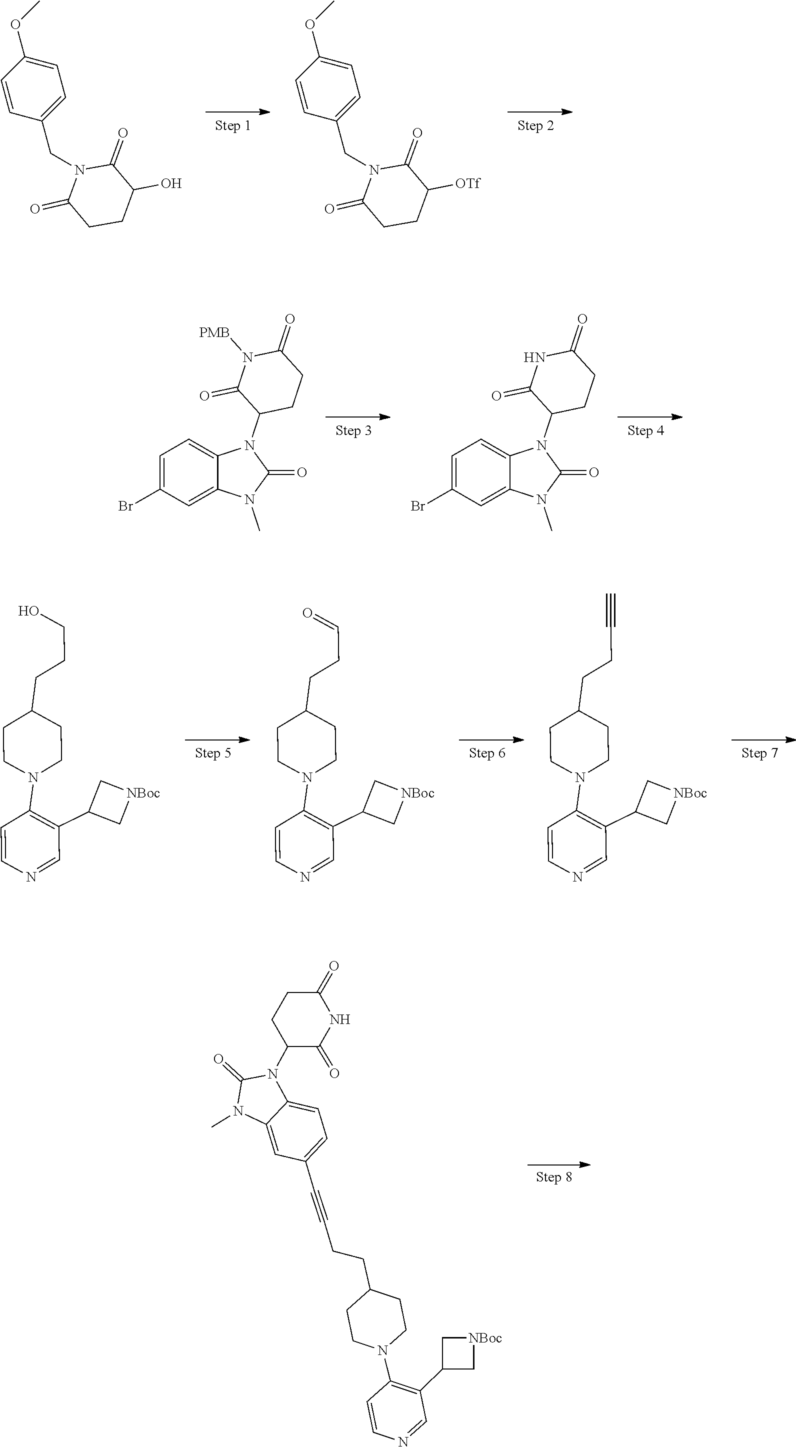

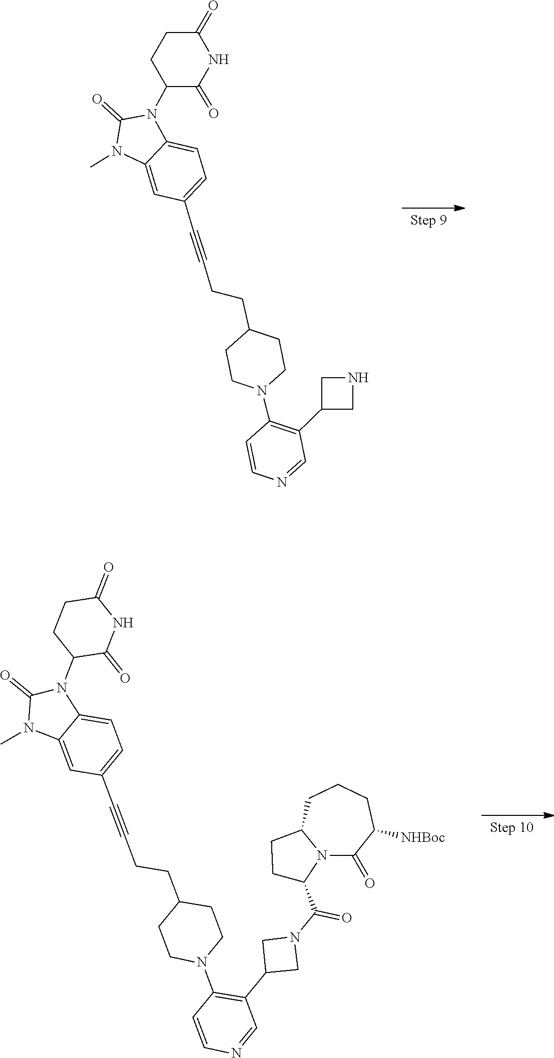 [SMILES: Cn1c(=O)n(C2CCC(=O)NC2=O)c2ccc(C#CCCC3CCN(c4ccncc4C4CNC4)CC3)cc21 ; Cn1c(=O)n(C2CCC(=O)NC2=O)c2ccc(C#CCCC3CCN(c4ccncc4C4CN(C(=O)[C@@H]5CC[C@@H]6CCC[C@H](NC(=O)OC(C)(C)C)C(=O)N65)C4)CC3)cc21]

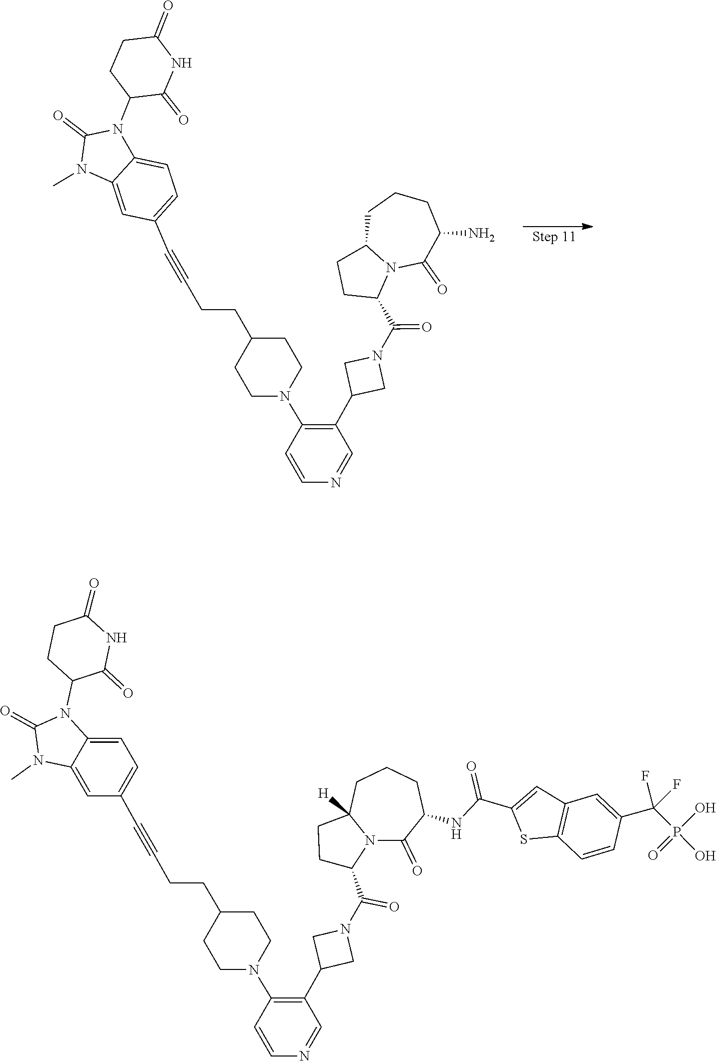 [SMILES: Cn1c(=O)n(C2CCC(=O)NC2=O)c2ccc(C#CCCC3CCN(c4ccncc4C4CN(C(=O)[C@@H]5CC[C@@H]6CCC[C@H](N)C(=O)N65)C4)CC3)cc21 ; Cn1c(=O)n(C2CCC(=O)NC2=O)c2ccc(C#CCCC3CCN(c4ccncc4C4CN(C(=O)[C@@H]5CC[C@@H]6CCC[C@H](NC(=O)c7cc8cc(C(F)(F)P(=O)(O)O)ccc8s7)C(=O)N65)C4)CC3)cc21]

### Step 1: Preparation of 1-(4-methoxybenzyl)-2,6-dioxopiperidin-3-yl trifluoromethanesulfonate

A solution of 3-hydroxy-1-(4-methoxybenzyl)piperidine-2,6-dione (1 g, 4.01 mmol, 1 eq), pyridine (634 mg, 8.02 mmol, 2 eq) in DCM (1 mL) was added trifluoromethanesulfonic anhydride (1.69 g, 6.01 mmol, 1.5 eq) at 0° C. The mixture was stirred at 0° C. for 0.5 hour. The mixture was concentrated under reduced pressure to give a residue. The residue was purified by flash silica gel chromatography to give 1-(4-methoxybenzyl)-2,6-dioxopiperidin-3-yl trifluoromethanesulfonate (1.49 g, 3.90 mmol, 98%) as a yellow oil. ¹H NMR (400 MHz, CHLOROFORM-d) δ 7.36 (d, J=8.8 Hz, 2H), 6.85-6.81 (m, 2H), 5.32-5.30 (m, 1H), 4.90 (s, 2H), 3.79 (s, 3H), 3.02-2.96 (m, 1H), 2.78-2.69 (m, 1H), 2.43-2.31 (m, 2H).

### Step 2: Preparation of 3-(5-bromo-3-methyl-2-oxo-2,3-dihydro-1H-benzo[d]imidazol-1-yl)-1-(4-methoxybenzyl)piperidine-2,6-dione

A solution of 6-bromo-1-methyl-1,3-dihydro-2H-benzo[d]imidazol-2-one (356 mg, 1.57 mmol, 1.2 eq), potassium tert-butoxide (293 mg, 2.62 mmol, 2 eq) in THF (1 mL) was added 1-(4-methoxybenzyl)-2,6-dioxopiperidin-3-yl trifluoromethanesulfonate (500 mg, 1.31 mmol, 1 eq) at 0° C. The mixture was stirred at 25° C. for 0.5 hour. The reaction mixture was concentrated under reduced pressure to give a residue. The residue was purified by flash silica gel chromatography to give a yellow solid, the solid was triturated with methanol, then filtered to give a white solid to give 3-(5-bromo-3-methyl-2-oxo-2,3-dihydro-1H-benzo[d]imidazol-1-yl)-1-(4-methoxybenzyl)piperidine-2,6-dione (135 mg, 294 μmol, 22.5% yield) as a white solid. ¹H NMR (400 MHz, DMSO-d₆) δ 7.47 (d, J=1.6 Hz, 1H), 7.23-7.15 (m, 3H), 7.00 (d, J=8.4 Hz, 1H), 6.88-6.82 (m, 2H), 5.55-5.51 (m, 1H), 4.84-4.73 (m, 2H), 3.72 (s, 3H), 3.34 (s, 3H), 3.09-2.98 (m, 1H), 2.86-2.65 (m, 3H).

### Step 3: Preparation of 3-(5-bromo-3-methyl-2-oxo-2,3-dihydro-1H-benzo[d]imidazol-1-yl)piperidine-2,6-dione

A solution of 3-(5-bromo-3-methyl-2-oxo-2,3-dihydro-1H-1,3-benzodiazol-1-yl)-1-[(4-methox-yphenyl)methyl]piperidine-2,6-dione (50 mg, 109 μmol, 1 eq) in toluene (1 mL) was added me-thanesulfonic acid (0.30 mL, 109 μmol, 1 eq). The mixture was stirred at 110° C. for 2 hours. The reaction mixture was concentrated under reduced pressure to give a residue. The residue was purified by flash silica gel chromatography to give 3-(5-bromo-3-methyl-2-oxo-2,3-dihydro-1H-benzo[d]imidazol-1-yl)piperidine-2,6-dione (20 mg, 59 μmol, 54% yield) a white solid. LCMS (ESI) m/z=337.9

### Step 4: Preparation of tert-butyl 3-(4-(4-(3-hydroxypropyl)piperidin-1-yl)pyridin-3-yl)azetidine-1-carboxylate

A solution of tert-butyl 3-(4-fluoropyridin-3-yl)azetidine-1-carboxylate (1 g, 3.96 mmo, 1.0 eq), cesium carbonate (3.87 g, 11.9 mmol, 3.5 eq) in DMSO (10 mL) was added 3-(piperidin-4-yl)propan-1-ol (0.85 g, 5.95 mmol, 1.5 eq) at 12 hours. The residue was diluted with water (20 mL) and extracted with ethyl acetate (30 mL×3). The combined organic layers were washed with brine (30 mL×3), dried over anhydrous Na₂SO₄, filtered and concentrated under reduced pressure to give a residue. The residue was purified by flash silica gel chromatography to give tert-butyl 3-(4-(4-(3-hydroxypropyl)piperidin-1-yl)pyridin-3-yl)azetidine-1-carboxylate (962 mg, 2.56 mmol, 64% yield) was obtained as a yellow solid.LCMS (ESI) m/z=376.3

### Step 5: Preparation of tert-butyl 3-(4-(4-(3-oxopropyl)piperidin-1-yl)pyridin-3-yl)azetidine-1-carboxylate

A solution of tert-butyl 3-{4-[4-(3-hydroxypropyl)piperidin-1-yl]pyridin-3-yl}azetidine-1-carboxylate (200 mg, 532 μmol, 1 eq) in DCM (2 mL) was added 1,1-bis(acetyloxy)-3-oxo-llambda5,2-benziodaoxol-1-yl acetate (248 mg, 585 μmol, 1.1 eq) at 0° C. The mixture was stirred at 25° C. for 1 hour. The reaction mixture was poured into saturation Na₂S₂O₃ (6 mL) aqueous solution and stirred at 25° C. for 0.5 hour. Then separated, the organic layer was washed with NaHCO₃ (4 mL×3), followed by brine (6 mL), then dried over Na₂SO₄ and concentrated. No further purification. tert-butyl 3-(4-(4-(3-oxopropyl)piperidin-1-yl)pyridin-3-yl)azetidine-1-carboxylate (215 mg, 575 μmol, crude) was obtained as a yellow solid. LCMS (ESI) m/z=374.1

### Step 6: Preparation of tert-butyl 3-(4-(4-(but-3-yn-1-yl)piperidin-1-yl)pyridin-3-yl)azetidine-1-carboxylate

A solution of tert-butyl 3-{4-[4-(3-oxopropyl)piperidin-1-yl]pyridin-3-yl}azetidine-1-carboxylate (215 mg, 575 μmol, 1 eq), potassium carbonate (158 mg, 1.15 mmol, 2 eq) in MeOH (2 mL) was added dimethyl (1-diazo-2-oxopropyl)phosphonate (121 mg, 632 μmol, 1.1 eq) at 0° C. The mixture was stirred at 90° C. for 9 hours. The residue was purified by flash silica gel chromatography to give tert-butyl 3-{4-[4-(but-3-yn-1-yl)piperidin-1-yl]pyridin-3-yl}azetidine-1-carboxylate (109 mg, 294 μmol, 51% yield) as a white oil. ¹H NMR (400 MHz, CHLOROFORM-d) δ 8.52 (s, 1H), 8.36 (d, J=5.6 Hz, 1H), 6.87 (d, J=5.6 Hz, 1H), 4.43-4.32 (m, 2H), 4.08-4.01 (m, 2H), 4.00-3.92 (m, 1H), 3.14 (d, J=12.0 Hz, 2H), 2.73 (t, J=11.6 Hz, 2H), 2.30-2.26 (m, 2H), 1.98 (t, J=2.8 Hz, 1H), 1.85 (d, J=11.6 Hz, 2H), 1.58-1.53 (m, 2H), 1.48 (s, 9H), 1.42-1.31 (m, 3H).

### Step 7: Preparation of tert-butyl 3-(4-(4-(4-(1-(2,6-dioxopiperidin-3-yl)-3-methyl-2-oxo-2,3-dihydro-1H-benzo[d]imidazol-5-yl)but-3-yn-1-yl)piperidin-1-yl)pyridin-3-yl)azetidine-1-carboxylate

A solution of tert-butyl 3-(4-(4-(but-3-yn-1-yl)piperidin-1-yl)pyridin-3-yl)azetidine-1-carboxylate (63 mg, 170 μmol, 1 eq) in DMSO (0.1 mL) was added 3-(5-bromo-3-methyl-2-oxo-2,3-dihydro-1H-benzo[d]imidazol-1-yl)piperidine-2,6-dione (57.40 mg, 170 μmol, 1 eq), copper iodide (1.68 mg, 17 μmol, 0.1 eq), palladium bis(triphenylphosphine) dichloride (12 mg, 17 μmol, 0.1 eq). The mixture was stirred at 80° C. for 5 hours. The residue was diluted with water (5 mL) and extracted with ethyl acetate (10 mL×3). The combined organic layers were washed with brine (10 mL×3), dried over anhydrous Na₂SO₄, filtered and concentrated under reduced pressure to give a residue. The residue was purified by flash silica gel chromatography to give tert-butyl 3-(4-(4-(4-(1-(2,6-dioxopiperidin-3-yl)-3-methyl-2-oxo-2,3-dihydro-1H-benzo[d]imidazol-5-yl)but-3-yn-1-yl)piperidin-1-yl)pyridin-3-yl)azetidine-1-carboxylate (50 mg, 80 μmol, 47% yield) as a yellow solid. LCMS (ESI) m/z=627.5. ¹H NMR (400 MHz, CHLOROFORM-d) δ 8.11 (s, 1H), 7.71-7.44 (m, 1H), 7.24-7.12 (m, 2H), 7.07 (s, 1H), 6.74-6.67 (m, 1H), 5.24-5.13 (m, 1H), 4.46-4.34 (m, 2H), 4.09-4.03 (m, 1H), 3.43 (s, 3H), 3.11-3.05 (m, 2H), 3.00-2.65 (m, 5H), 2.49 (t, J=6.4 Hz, 2H), 2.37-2.19 (m, 2H), 1.93-1.75 (m, 2H), 1.64 (s, 3H), 1.48 (s, 9H), 1.44-1.33 (m, 3H).

### Step 8: Preparation of 3-(5-(4-(1-(3-(azetidin-3-yl)pyridin-4-yl)piperidin-4-yl)but-1-yn-1-yl)-3-methyl-2-oxo-2,3-dihydro-1H-benzo[d]imidazol-1-yl)piperidine-2,6-dione

A solution of tert-butyl 3-(4-(4-(4-(1-(2,6-dioxopiperidin-3-yl)-3-methyl-2-oxo-2,3-dihydro-1H-benzo[d]imidazol-5-yl)but-3-yn-1-yl)piperidin-1-yl)pyridin-3-yl)azetidine-1-carboxylate (40 mg, 64 μmol, 1 eq) in DCM (0.60 mL) was added TFA (0.20 mL). The mixture was stirred at 25° C. for 0.5 hour. The mixture was concentrated under reduced pressure to give 3-(5-(4-(1-(3-(azetidin-3-yl)pyridin-4-yl)piperidin-4-yl)but-1-yn-1-yl)-3-methyl-2-oxo-2,3-dihydro-1H-benzo[d]imidazol-1-yl)piperidine-2,6-dione (33 mg, crude) as a yellow oil.

### Step 9: Preparation of tert-butyl ((3S,6S,9aS)-3-(3-(4-(4-(4-(1-(2,6-dioxopiperidin-3-yl)-3-methyl-2-oxo-2,3-dihydro-1H-benzo[d]imidazol-5-yl)but-3-yn-1-yl)piperidin-1-yl)pyridin-3-yl)azetidine-1-carbonyl)-5-oxooctahydro-1H-pyrrolo[1,2-a]azepin-6-yl)carbamate

A solution of (3S,6S,9aS)-6-((tert-butoxycarbonyl)amino)-5-oxooctahydro-1H-pyrrolo[1,2-a]azepine-3-carboxylic acid (16 mg, 52 μmol, 1 eq), ethylbis(propan-2-yl)amine (36 μL, 208 μmol, 4 eq) in DMF (0.50 mL) was added 1-[(dimethylamino)(dimethyliminio)methyl]-3-oxo-2H,3H-3lambda5-[1,2,3]triazolo[5,4-b]pyridin-3-ylium-2-ide; hexafluoro-lambda5-phosphanuide (24 mg, 63 μmol, 1.2 eq). The mixture was stirred at 25° C. for 10 minutes. Then was added 3-(5-(4-(1-(3-(azetidin-3-yl)pyridin-4-yl)piperidin-4-yl)but-1-yn-1-yl)-3-methyl-2-oxo-2,3-dihydro-1H-benzo[d]imidazol-1-yl)piperidine-2,6-dione (33 mg, 63 μmol, 1.2 eq), ethylbis(propan-2-yl)amine (36 μL, 208 μmol, 4 eq) in DMF (0.50 mL). The mixture was stirred at 25° C. for 20 minutes. The mixture was diluted with water (3 mL) and extracted with ethyl acetate (3 mL×3). The combined organic layers were washed with brine (3 mL×3), dried over anhydrous Na₂SO₄, filtered and concentrated under reduced pressure to give a residue. The residue was purified by flash silica gel chromatography to give tert-butyl N-[(3S,6S,9aS)-3-{3-[4-(4-{4-[1-(2,6-dioxopiperidin-3-yl)-3-methyl-2-oxo-2,3-dihydro-1H-1,3-benzodiazol-5-yl]but-3-yn-1-yl}piperidin-1-yl)pyridin-3-yl]azetidine-1-carbonyl}-5-oxo-octahydro-1H-pyrrolo[1,2-a]azepin-6-yl]carbamate (50 mg, impurity) as a yellow solid. LCMS (ESI) m/z=821.4

### Step 10: Preparation of 3-(5-(4-(1-(3-(1-((3S,6S,9aS)-6-amino-5-oxooctahydro-1H-pyrrolo[1,2-a]azepine-3-carbonyl)azetidin-3-yl)pyridin-4-yl)piperidin-4-yl)but-1-yn-1-yl)-3-methyl-2-oxo-2,3-dihydro-1H-benzo[d]imidazol-1-yl)piperidine-2,6-dione

A solution of tert-butyl N-[(3S,6S,9aS)-3-{3-[4-(4-{4-[1-(2,6-dioxopiperidin-3-yl)-3-methyl-2-oxo-2,3-dihydro-1H-1,3-benzodiazol-5-yl]but-3-yn-1-yl}piperidin-1-yl)pyridin-3-yl]azetidine-1-carbonyl}-5-oxo-octahydro-1H-pyrrolo[1,2-a]azepin-6-yl]carbamate (25 mg, 30 μmol, 1 eq) in DCM (0.30 mL) was added TFA (0.10 mL, 30 μmol, 1 eq). The mixture was stirred at 25° C. for 0.5 hour. The mixture was concentrated under reduced pressure to give crude 3-(5-{4-[1-(3-{1-[(3S,6S,9aS)-6-amino-5-oxo-octahydro-1H-pyrrolo[1,2-a]azepine-3-carbonyl]azetidin-3-yl}pyridin-4-yl)piperidin-4-yl]but-1-yn-1-yl}-3-methyl-2-oxo-2,3-dihydro-1H-1,3-benzodiazol-1-yl)piperidine-2,6-dione (22 mg, crude) as a yellow oil.

### Step 11: Preparation of ((2-(((3S,6S,9aS)-3-(3-(4-(4-(4-(1-(2,6-dioxopiperidin-3-yl)-3-methyl-2-oxo-2,3-dihydro-1H-benzo[d]imidazol-5-yl)but-3-yn-1-yl)piperidin-1-yl)pyridin-3-yl)azetidine-1-carbonyl)-5-oxooctahydro-1H-pyrrolo[1,2-a]azepin-6-yl)carbamoyl)benzo[b]thiophen-5-yl)difluoromethyl)phosphonic acid

A solution of 3-(5-{4-[1-(3-{1-[(3S,6S,9aS)-6-amino-5-oxo-octahydro-1H-pyrrolo[1,2-a]azepine-3-carbonyl]azetidin-3-yl}pyridin-4-yl)piperidin-4-yl]but-1-yn-1-yl}-3-methyl-2-oxo-2,3-dihydr-o-1H-1,3-benzodiazol-1-yl)piperidine-2,6-dione (22 mg, 31 μmol, 1.2 eq) in DMF (0.20 mL) was added ethylbis(propan-2-yl)amine (18 pL, 101 μmol, 4 eq), (difluoro(2-((perfluorophenoxy)carbonyl)benzo[b]thiophen-5-yl)methyl)phosphonic acid (12 mg, 25 μmol, 1 eq). The mixture was stirred at 25° C. for 0.5 hour. The mixture was purified by prep-HPLC (Column: Welch Xtimate C18 150×25 mm×5 um; water (TFA)-ACN]B %: 20%-50%, 10 min) to give ((2-(((3S,6S,9aS)-3-(3-(4-(4-(4-(1-(2,6-dioxopiperidin-3-yl)-3-methyl-2-oxo-2,3-dihydro-1H-benzo[d]imidazol-5-yl)but-3-yn-1-yl)piperidin-1-yl)pyridin-3-yl)azetidine-1-carbon-yl)-5-oxooctahydro-1H-pyrrolo[1,2-a]azepin-6-yl)carbamoyl)benzo[b]thiophen-5-yl)difluorome-thyl)phosphonic acid (Compound 34, 3.90 mg, 15% yield) as a white solid. LCMS (ESI) m/z=1011.4. ¹H NMR (400 MHz, DMSO-d₆) δ 11.24-11.03 (m, 1H), 8.86-8.61 (m, 1H), 8.51-8.26 (m, 1H), 8.23-7.93 (m, 4H), 7.63-7.44 (m, 1H), 7.21 (d, J=19.6 Hz, 1H), 7.10-6.88 (m, 3H), 5.43-5.31 (m, 1H), 4.77-4.57 (m, 2H), 4.50-4.30 (m, 2H), 4.27-4.07 (m, 2H), 4.00-3.92 (m, 2H), 3.40 (s, 1H), 3.32 (s, 1H), 2.97-2.81 (m, 4H), 2.76-2.68 (m, 1H), 2.64-2.57 (m, 2H), 2.23-2.09 (m, 2H), 2.07-1.91 (m, 4H), 1.86-1.61 (m, 11H), 1.59-1.42 (m, 3H), 1.36-1.18 (m, 3H).

### Preparation of S,S(((((2-(((3S,6S,9aS)-3-(3-(4-(4-(4-(1-(2,6-dioxopiperidin-3-yl)-3-methyl-2-oxo-2,3-dihydro-1H-benzo[d]imidazol-5-yl)but-3-yn-1-yl)piperidin-1-yl)pyridin-3-yl)azetidine-1-carbonyl)-5-oxooctahydro-1H-pyrrolo[1,2-a]azepin-6-yl)carbamoyl)benzo[b]thiophen-5-yl)difluoromethyl)phosphoryl)bis(oxy))bis(ethane-2,1-diyl)) bis(2,2-dimethylpropanethioate) (Compound 35)

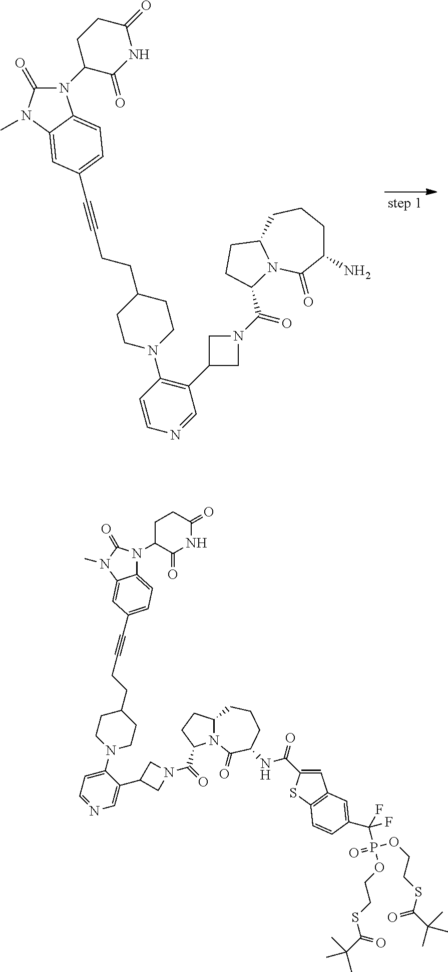 [SMILES: Cn1c(=O)n(C2CCC(=O)NC2=O)c2ccc(C#CCCC3CCN(c4ccncc4C4CN(C(=O)[C@@H]5CC[C@@H]6CCC[C@H](N)C(=O)N65)C4)CC3)cc21 ; Cn1c(=O)n(C2CCC(=O)NC2=O)c2ccc(C#CCCC3CCN(c4ccncc4C4CN(C(=O)[C@@H]5CC[C@@H]6CCC[C@H](NC(=O)c7cc8cc(C(F)(F)P(=O)(OCCSC(=O)C(C)(C)C)OCCSC(=O)C(C)(C)C)ccc8s7)C(=O)N65)C4)CC3)cc21]

### Step 1: Preparation of S,S(((((2-(((3S,6S,9aS)-3-(3-(4-(4-(4-(1-(2,6-dioxopiperidin-3-yl)-3-methyl-2-oxo-2,3-dihydro-1H-benzo[d]imidazol-5-yl)but-3-yn-1-yl)piperidin-1-yl)pyridin-3-yl)azetidine-1-carbonyl)-5-oxooctahydro-1H-pyrrolo[1,2-a]azepin-6-yl)carbamoyl)benzo[b]thiophen-5-yl)difluoromethyl)phosphoryl)bis(oxy))bis(ethane-2,1-diyl)) bis(2,2-dimethylpropanethioate)

To a solution of 4-nitrophenyl 5-{[bis({2-[(2,2-dimethylpropanoyl)sulfanyl]ethoxy})phosphoryl]difluoromethyl}-1-benzothiophene-2-carboxylate (80 mg, 0.11 mmol, 1 eq) and 3-(5-{4-[1-(3-{1-[(3S,6S,9aS)-6-amino-5-oxo-octahydro-1H-pyrrolo[1,2-a]azepine-3-carbonyl]azetidin-3-yl}pyridin-4-yl)piperidin-4-yl]but-1-yn-1-yl}-3-methyl-2-oxo-2,3-dihydro-1H-1,3-benzodiazol-1-yl)piperidine-2,6-dione (0.10 g, 0.14 mmol, 1.3 eq) in DMF (1 mL) was added hydroxybenzotriazole (22 mg, 0.17 mmol, 1.5 eq) and N,N-diisopropylethylamine (29 mg, 0.22 mmol, 2 eq) was stirred at 25° C. for 20 minutes to give a brown solution. The reaction mixture was filtered and the filtrate was purified by prep-HPLC (column: Waters xbridge 150×25 mm 10 um; mobile phase: [water (NH₄HCO₃)-ACN]; B %: 58%-78%, 14 min) then lyophilization to give S,S [symbol] (((((2-(((3S,6S,9aS)-3-(3-(4-(4-(4-(1-(2,6-dioxopiperidin-3-yl)-3-methyl-2-oxo-2,3-dihydro-1H-benzo[d]imidazol-5-yl)but-3-yn-1-yl)piperidin-1-yl)pyridin-3-yl)azetidine-1-carbonyl)-5-oxooctahydro-1H-pyrrolo[1,2-a]azepin-6-yl)carbamoyl)benzo[b]thiophen-5-yl)difluoromethyl)phosphoryl)bis(oxy))bis(ethane-2,1-diyl)) bis(2,2-dimethylpropanethioate) (Compound 35, 34 mg, 26 mol, 24% yield) as a white solid. LCMS (ESI) m/z=1299.8. ¹H NMR (400 MHz, METHANOL-d₄) δ 8.58-8.41 (m, 1H), 8.27 (t, J=5.6 Hz, 1H), 8.17 (d, J=4.0 Hz, 2H), 8.12-8.03 (m, 1H), 7.64 (d, J=8.8 Hz, 1H), 7.22-7.09 (m, 2H), 7.04 (dd, J=3.2, 8.0 Hz, 2H), 5.32 (dd, J=5.2, 12.4 Hz, 1H), 4.79-4.73 (m, 2H), 4.69-4.61 (m, 1H), 4.61-4.52 (m, 2H), 4.48-4.35 (m, 1H), 4.27-4.14 (m, 5H), 4.13-4.02 (m, 2H), 3.40 (d, J=2.8 Hz, 3H), 3.24-3.18 (m, 2H), 3.16-3.10 (m, 4H), 2.94-2.87 (m, 1H), 2.83-2.71 (m, 4H), 2.54-2.44 (m, 2H), 2.35-2.26 (m, 1H), 2.21-2.07 (m, 3H), 2.03-1.95 (m, 3H), 1.91 (d, J=10.4 Hz, 3H), 1.84 (d, J=4.0 Hz, 2H), 1.70-1.59 (m, 3H), 1.45 (dd, J=1.6, 7.2 Hz, 2H), 1.19 (s, 18H).

### Preparation of ((2-(((3S,6S,9aS)-3-(3-(4-(4-(4-(1-(2,6-dioxopiperidin-3-yl)-3-methyl-2-oxo-2,3-dihydro-1H-benzo[d]imidazol-5-yl)but-3-yn-1-yl)piperidin-1-yl)pyridin-3-yl)azetidine-1-carbonyl)-5-oxooctahydro-1H-pyrrolo[1,2-a]azepin-6-yl)carbamoyl)benzo[b]thiophen-5-yl)fluoromethyl)phosphonic acid (Compound 36)

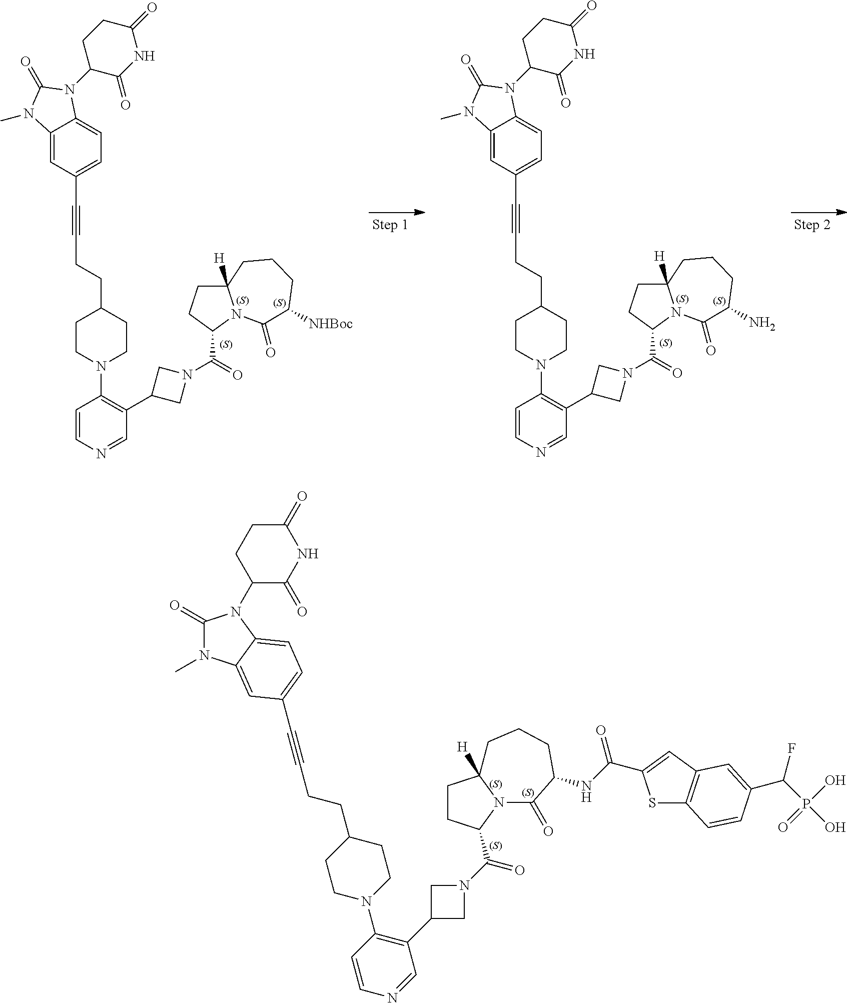

### Step 1: Preparation of 3-(5-(4-(1-(3-(1-((3S,6S,9aS)-6-amino-5-oxooctahydro-1H-pyrrolo[1,2-a]azepine-3-carbonyl)azetidin-3-yl)pyridin-4-yl)piperidin-4-yl)but-1-yn-1-yl)-3-methyl-2-oxo-2,3-dihydro-1H-benzo[d]imidazol-1-yl)piperidine-2,6-dione

To a solution of tert-butyl ((3S,6S,9aS)-3-(3-(4-(4-(4-(1-(2,6-dioxopiperidin-3-yl)-3-methyl-2-oxo-2,3-dihydro-1H-benzo[d]imidazol-5-yl)but-3-yn-1-yl)piperidin-1-yl)pyridin-3-yl)azetidine-1-carbonyl)-5-oxooctahydro-1H-pyrrolo[1,2-a]azepin-6-yl)carbamate (12 mg, 14 μmol, 1.0 eq) in DCM (0.15 mL) was added trifluoroacetic acid (0.05 mL). The mixture was stirred at 25° C. for 0.5 hour. The reaction mixture was concentrated under reduced pressure to give 3-(5-(4-(1-(3-(1-((3S,6S,9aS)-6-amino-5-oxooctahydro-1H-pyrrolo[1,2-a]azepine-3-carbonyl)azetidin-3-yl)pyridin-4-yl)piperidin-4-yl)but-1-yn-1-yl)-3-methyl-2-oxo-2,3-dihydro-1H-benzo[d]imidazol-1-yl)piperidine-2,6-dione (13.0 mg, crude) was used into the next step without further purification.

### Step 2: Preparation of ((2-(((3S,6S,9aS)-3-(3-(4-(4-(4-(1-(2,6-dioxopiperidin-3-yl)-3-methyl-2-oxo-2,3-dihydro-1H-benzo[d]imidazol-5-yl)but-3-yn-1-yl)piperidin-1-yl)pyridin-3-yl)azetidine-1-carbonyl)-5-oxooctahydro-1H-pyrrolo[1,2-a]azepin-6-yl)carbamoyl)benzo[b]thiophen-5-yl)fluoromethyl)phosphonic acid

A mixture of 3-(5-(4-(1-(3-(1-((3S,6S,9aS)-6-amino-5-oxooctahydro-1H-pyrrolo[1,2-a]azepine-3-carbonyl)azetidin-3-yl)pyridin-4-yl)piperidin-4-yl)but-1-yn-1-yl)-3-methyl-2-oxo-2,3-dihydro-1H-benzo[d]imidazol-1-yl)piperidine-2,6-dione (9.37 mg, 13.0 μmol, 1.2 eq) and ethylbis(propan-2-yl)amine (5.63 mg, 43.6 μmol, 4.0 eq) in DMF (0.1 mL). The mixture was neutralized to pH=7-8 with DIEA then stirred at 25° C. for 5 minutes. To a solution of (fluoro(2-((perfluorophenoxy)carbonyl)benzo[b]thiophen-5-yl)methyl)phosphonic acid (5.0 mg, 11 μmol, 1 eq) in DMF (0.1 mL) was added to the mixture and stirred at 25° C. for 15 minutes. The reaction mixture was purified by prep-HPLC (column: Welch Xtimate C18 150×25 mm×5 um; mobile phase: [water (FA)-ACN]; B %: 20%-50%, 10 min) then lyophilization to give ((2-(((3S,6S,9aS)-3-(3-(4-(4-(4-(1-(2,6-dioxopiperidin-3-yl)-3-methyl-2-oxo-2,3-dihydro-1H-benzo[d]imidazol-5-yl)but-3-yn-1-yl)piperidin-1-yl)pyridin-3-yl)azetidine-1-carbonyl)-5-oxooctahydro-1H-pyrrolo[1,2-a]azepin-6-yl)carbamoyl)benzo[b]thiophen-5-yl)fluoromethyl)phosphonic acid (Compound 36, 1.50 mg, 1.51 μmol, 13.8% yield) as a white solid. LCMS (ESI) m/z=993.3.

¹H NMR (400 MHz, DMSO-d₆) δ 11.11 (s, 1H), 8.76-8.66 (m, 1H), 8.49-8.27 (m, 2H), 8.22 (d, J=12.4 Hz, 1H), 8.02 (dd, J=3.2, 8.4 Hz, 1H), 7.98-7.93 (m, 1H), 7.52 (d, J=8.0 Hz, 1H), 7.24-7.13 (m, 2H), 7.10-7.05 (m, 2H), 5.89-5.72 (m, 1H), 5.42-5.32 (m, 1H), 4.82-4.62 (m, 2H), 4.43-4.32 (m, 2H), 4.31-4.25 (m, 1H), 4.18-4.05 (m, 2H), 3.99-3.93 (m, 2H), 3.33 (s, 3H), 3.00-2.95 (m, 2H), 2.89-2.86 (m, 1H), 2.68-2.62 (m, 2H), 2.36-2.28 (m, 1H), 2.22-2.16 (m, 1H), 2.05-1.93 (m, 4H), 1.87-1.78 (m, 6H), 1.75-1.65 (m, 5H), 1.61-1.49 (m, 3H), 1.34-1.21 (m, 3H).

### Preparation of ((1R)-(2-(((3S,6S,9aS)-3-(3-(4-(4-(4-(1-(2,6-dioxopiperidin-3-yl)-3-methyl-2-oxo-2,3-dihydro-1H-benzo[d]imidazol-5-yl)but-3-yn-1-yl)piperidin-1-yl)pyridin-3-yl)azetidine-1-carbonyl)-5-oxooctahydro-1H-pyrrolo[1,2-a]azepin-6-yl)carbamoyl)benzo[b]thiophen-5-yl)fluoromethyl)phosphonic acid (Compound 37)

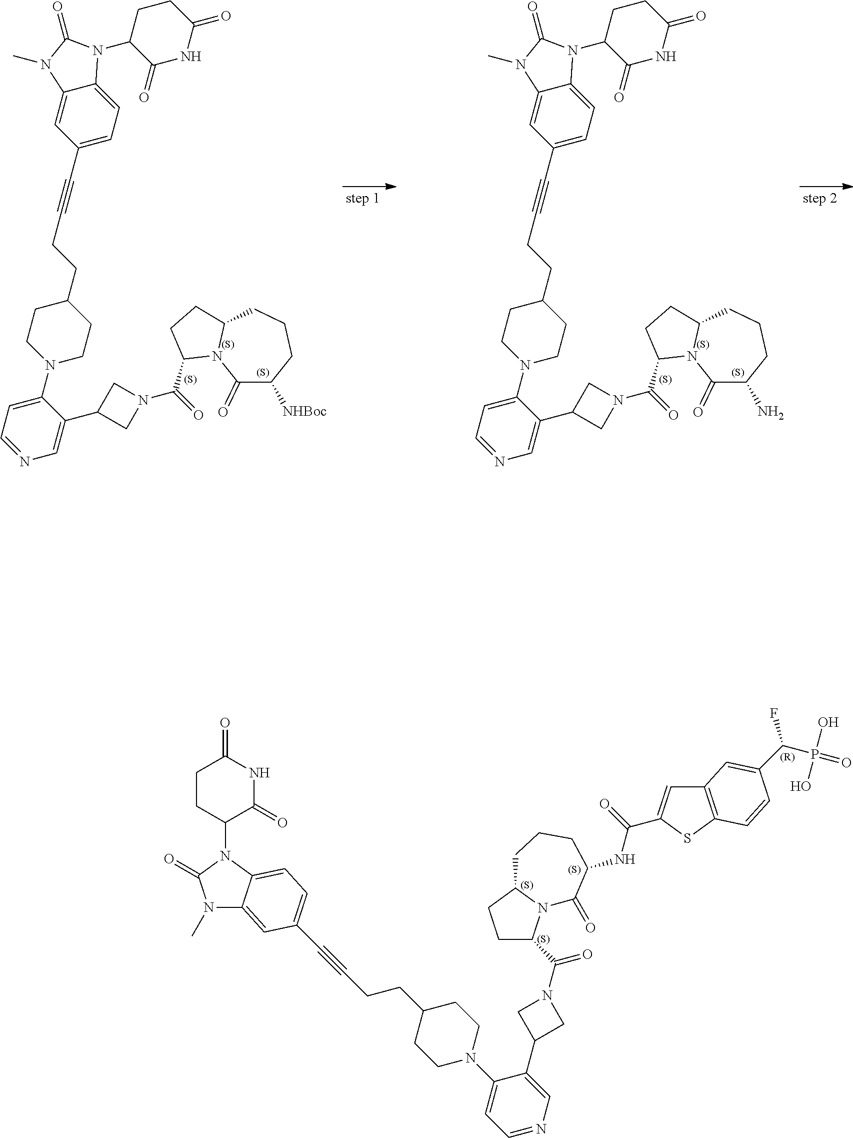

### Step 1: 3-(5-(4-(1-(3-(1-((3S,6S,9aS)-6-amino-S-oxooctahydro-1H-pyrrolo[1,2-a]azepine-3-carbonyl)azetidin-3-yl)pyridin-4-yl)piperidin-4-yl)but-1-yn-1-yl)-3-methyl-2-oxo-2,3-dihydro-1H-benzo[d]imidazol-1-yl)piperidine-2,6-dione

A mixture of tert-butyl N-[(3S,6S,9aS)-3-{3-[4-(4-{4-[1-(2,6-dioxopiperidin-3-yl)-3-methyl-2-oxo-2,3-dihydro-1H-1,3-benzodiazol-5-yl]but-3-yn-1-yl}piperidin-1-yl)pyridin-3-yl]azetidine-1-carbonyl}-5-oxo-octahydro-1H-pyrrolo[1,2-a]azepin-6-yl]carbamate (70 mg, 85 μmol, 1.0 eq) in methylene chloride (1.0 mL) was added trifluoroacetic acid (0.2 mL), then the mixture was stirred at 25° C. for 0.5 hours. The reaction was concentrated under reduced pressure to give 3-(5-{4-[1-(3-{1-[(3S,6S,9aS)-6-amino-5-oxo-octahydro-1H-pyrrolo[1,2-a]azepine-3-carbonyl]azetidin-3-yl}pyridin-4-yl)piperidin-4-yl]but-1-yn-1-yl}-3-methyl-2-oxo-2,3-dihydro-1H-1,3-benzodiazol-1-yl)piperidine-2,6-dione (60 mg, crude) was obtained as a yellow oil. LCMS (ESI) m/z=721.5

### Step 2: ((JR)-(2-(((3S,6S,9aS)-3-(3-(4-(4-(4-(1-(2,6-dioxopiperidin-3-yl)-3-methyl-2-oxo-2,3-dihydro-1H-benzo[d]imidazol-5-yl)but-3-yn-1-yl)piperidin-1-yl)pyridin-3-yl)azetidine-1-carbonyl)-5-oxooctahydro-1H-pyrrolo[1,2-a]azepin-6-yl)carbamoyl)benzo[b]thiophen-5-yl)fluoromethyl)phosphonic acid

A mixture of 3-(5-{4-[1-(3-{1-[(3S,6S,9aS)-6-amino-5-oxo-octahydro-1H-pyrrolo[1,2-a]azepine-3-carbonyl]azetidin-3-yl}pyridin-4-yl)piperidin-4-yl]but-1-yn-1-yl}-3-methyl-2-oxo-2,3-dihydro-1H-1,3-benzodiazol-1-yl)piperidine-2,6-dione (60 mg, 83 μmol, 1.0 eq) in dimethylformamide (1.0 mL) was added ethylbis(propan-2-yl)amine (32 mg, 0.25 mmol, 3.0 eq), [(R)-fluoro({2-[(2,3,4,5,6-pentafluorophenoxy)carbonyl]-1-benzothiophen-5-yl})methyl]phosphonic acid (30 mg, 67 μmol, 0.8 eq), and then the mixture was stirred at 25° C. for 0.2 hours. The reaction mixture was purified by reversed-phase HPLC (Column:Phenomenex luna C18 150×25 mm×10 um; Condition:water (FA)-ACN; Gradient Time (min):8) to give [(R)-(2-{[(3S,6S,9aS)-3-{3-[4-(4-{4-[1-(2,6-dioxopiperidin-3-yl)-3-methyl-2-oxo-2,3-dihydro-1H-1,3-benzodiazol-5-yl]but-3-yn-1-yl}piperidin-1-yl)pyridin-3-yl]azetidine-1-carbonyl}-5-oxo-octahydro-1H-pyrrolo[1,2-a]azepin-6-yl]carbamoyl}-1-benzothiophen-5-yl)(fluoro)methyl]phosphonic acid (Compound 37, 4.15 mg, 4.18 μmol, 5.0% yield) as a white solid. LCMS (ESI) m/z=993.5. ¹H NMR (400 MHz, DMSO-d₆) δ 11.11 (s, 1H), 8.71 (s, 1H), 8.47-8.30 (m, 1H), 8.16 (s, 2H), 7.88 (s, 2H), 7.47 (s, 1H), 7.23 (d, J=11.6 Hz, 1H), 7.09 (s, 2H), 6.97-6.79 (m, 1H), 5.72-5.54 (m, 1H), 5.41-5.32 (m, 1H), 4.64 (d, J=3.6 Hz, 2H), 4.52-4.32 (m, 3H), 4.26 (d, J=4.4 Hz, 1H), 4.08-4.02 (m, 1H), 3.95 (d, J=2.4 Hz, 3H), 3.32 (s, 4H), 3.18-3.08 (m, 3H), 2.95-2.82 (m, 2H), 2.71-2.62 (m, 4H), 2.17 (s, 1H), 2.00 (s, 3H), 1.85-1.73 (m, 7H), 1.55 (s, 3H), 1.39-1.10 (m, 3H).

### Preparation of propyl (((1R)-(2-(((3S,6S,9aS)-3-(3-(4-(4-(4-(1-(2,6-dioxopiperidin-3-yl)-3-methyl-2-oxo-2,3-dihydro-1H-benzo[d]imidazol-5-yl)but-3-yn-1-yl)piperidin-1-yl)pyridin-3-yl)azetidine-1-carbonyl)-5-oxooctahydro-1H-pyrrolo[1,2-a]azepin-6-yl)carbamoyl)benzo[b]thiophen-5-yl)fluoromethyl)(phenoxy)phosphoryl)-L-alaninate (Compound 38)

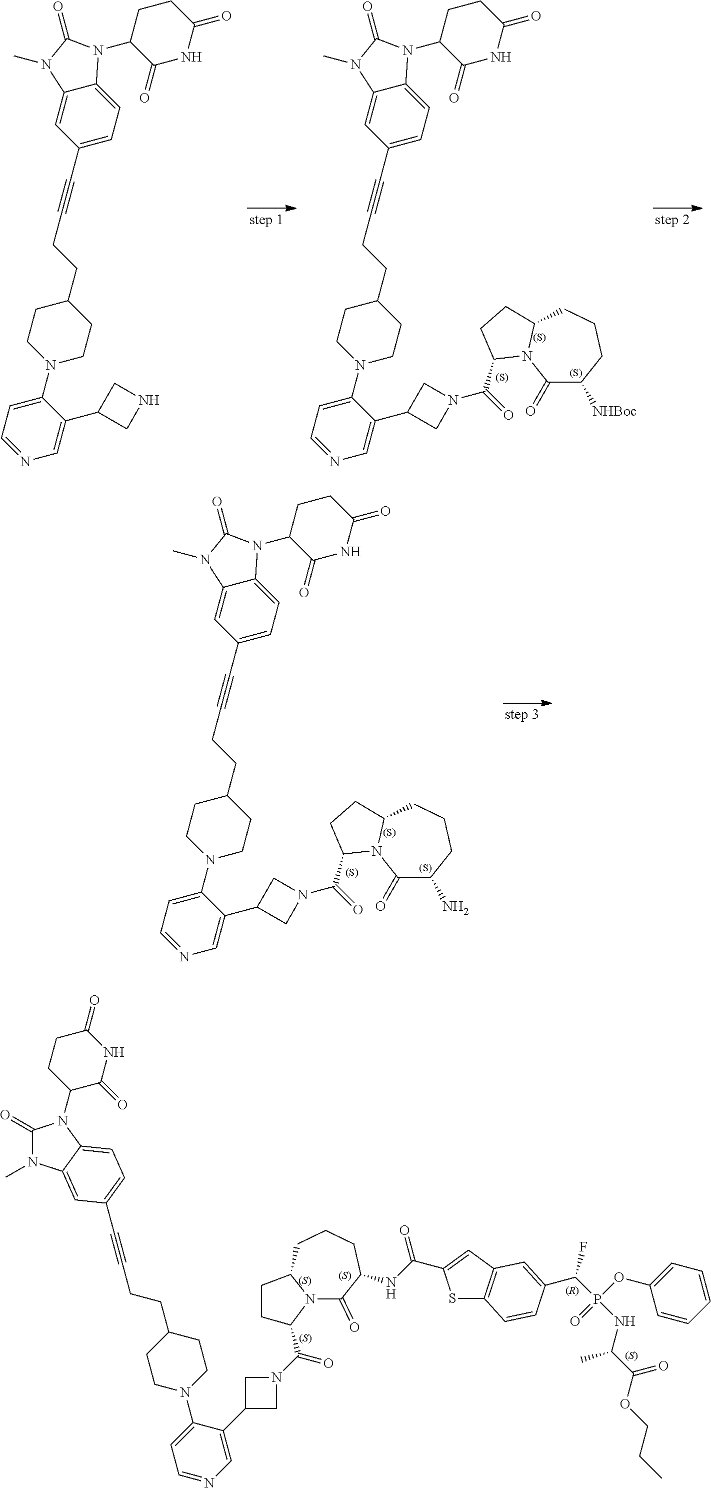

### Step 1: Preparation of tert-butyl ((3S,6S,9aS)-3-(3-(4-(4-(4-(1-(2,6-dioxopiperidin-3-yl)-3-methyl-2-oxo-2,3-dihydro-1H-benzo[d]imidazol-5-yl)but-3-yn-1-yl)piperidin-1-yl)pyridin-3-yl)azetidine-1-carbonyl)-5-oxooctahydro-1H-pyrrolo[1,2-a]azepin-6-yl)carbamate

Mixture 1 of 3-[5-(4-{1-[3-(azetidin-3-yl)pyridin-4-yl]piperidin-4-yl}but-1-yn-1-yl)-3-methyl-2-oxo-2,3-dihydro-1H-1,3-benzodiazol-1-yl]piperidine-2,6-dione (0.75 g, 1.4 mmol, 1.0 eq) in dimethylformamide (10 mL) was added ethylbis(propan-2-yl)amine to adjust the pH to 6-7, at the same time, a mixture 2of (3S,6S,9aS)-6-{[(tert-butoxy)carbonyl]amino}-5-oxo-octahydro-1H-pyrrolo[1,2-a]azepine-3-carboxylic acid (0.44 g, 1.4 mmol, 1.0 eq) and HATU (1.7 g, 4.4 mmol, 1.5 eq) in dimethylformamide (10 mL) was added ethylbis(propan-2-yl)amine to adjust the pH to 10, and stirred for 5 mins. And then mixture 1 was added to mixture 2 slowly. The reaction was stirred at 25° C. for 0.2 hours. The mixture was purified by reversed-phase (FA) to give compound tert-butyl N-[(3S,6S,9aS)-3-{3-[4-(4-{4-[1-(2,6-dioxopiperidin-3-yl)-3-methyl-2-oxo-2,3-dihydro-1H-1,3-benzodiazol-5-yl]but-3-yn-1-yl}piperidin-1-yl)pyridin-3-yl]azetidine-1-carbonyl}-5-oxo-octahydro-1H-pyrrolo[1,2-a]azepin-6-yl]carbamate (0.40 g, 0.49 mmol, 35% yield) as a white solid. LCMS (ESI) m/z=821.3

### Step 2: Preparation of 3-(5-(4-(1-(3-(1-((3S,6S,9aS)-6-amino-5-oxooctahydro-1H-pyrrolo[,2-a]azepine-3-carbonyl)azetidin-3-yl)pyridin-4-yl)piperidin-4-yl)but-1-yn-1-yl)-3-methyl-2-oxo-2,3-dihydro-1H-benzo[d]imidazol-1-yl)piperidine-2,6-dione

A mixture of tert-butyl N-[(3S,6S,9aS)-3-{3-[4-(4-{4-[1-(2,6-dioxopiperidin-3-yl)-3-methyl-2-oxo-2,3-dihydro-1H-1,3-benzodiazol-5-yl]but-3-yn-1-yl}piperidin-1-yl)pyridin-3-yl]azetidine-1-carbonyl}-5-oxo-octahydro-1H-pyrrolo[1,2-a]azepin-6-yl]carbamate (50 mg, 60 μmol, 1.0 eq) in methylene chloride (5.0 mL) was added trifluoroacetic acid (0.5 mL), then the mixture was stirred at 25° C. for 0.2 hours. The reaction mixture was concentrated under reduced pressure to give compound 3-(5-{4-[1-(3-{1-[(3S,6S,9aS)-6-amino-5-oxo-octahydro-1H-pyrrolo[1,2-a]azepine-3-carbonyl]azetidin-3-yl}pyridin-4-yl)piperidin-4-yl]but-1-yn-1-yl}-3-methyl-2-oxo-2,3-dihydro-1H-1,3-benzodiazol-1-yl)piperidine-2,6-dione (50 mg, crude) as a yellow oil. LCMS (ESI) m/z=721.5

### Step 3: Preparation of propyl (((JR)-(2-(((3S,6S,9aS)-3-(3-(4-(4-(4-(1-(2,6-dioxopiperidin-3-yl)-3-methyl-2-oxo-2,3-dihydro-1H-benzo[d]imidazol-5-yl)but-3-yn-1-yl)piperidin-1-yl)pyridin-3-yl)azetidine-1-carbonyl)-5-oxooctahydro-1H-pyrrolo[1,2-a]azepin-6-yl)carbamoyl)benzo[b]thiophen-5-yl)fluoromethyl)(phenoxy)phosphoryl)-L-alaninate

A mixture of 3-(5-{4-[1-(3-{1-[(3S,6S,9aS)-6-amino-5-oxo-octahydro-1H-pyrrolo[1,2-a]azepine-3-carbonyl]azetidin-3-yl}pyridin-4-yl)piperidin-4-yl]but-1-yn-1-yl}-3-methyl-2-oxo-2,3-dihydro-1H-1,3-benzodiazol-1-yl)piperidine-2,6-dione (50 mg, 69 μmol, 1.0 eq) in dimethylformamide (0.5 mL) was added ethylbis(propan-2-yl)amine to adjust the pH to 7, then added 2,3,4,5,6-pentafluorophenyl 5-[(R)-fluoro({[(2S)-1-oxo-1-propoxypropan-2-yl]amino}(phenoxy)phosphoryl)methyl]-1-benzothiophene-2-carboxylate (31 mg, 48 μmol, 0.7 eq), and the mixture was stirred at 25° C. for 0.2 hours. The mixture was purified by reversed-phase HPLC (Column: Phenomenex luna C18 150×25 mm×10 um; Condition: water (FA)-ACN; Begin B: 34; End B: 54; Gradient Time (min): 10) to give propyl (2S)-2-({[(R)-(2-{[(3S,6S,9aS)-3-{3-[4-(4-{4-[1-(2,6-dioxopiperidin-3-yl)-3-methyl-2-oxo-2,3-dihydro-1H-1,3-benzodiazol-5-yl]but-3-yn-1-yl}piperidin-1-yl)pyridin-3-yl]azetidine-1-carbonyl}-5-oxo-octahydro-1H-pyrrolo[1,2-a]azepin-6-yl]carbamoyl}-1-benzothiophen-5-yl)(fluoro)methyl](phenoxy)phosphoryl}amino)propanoate (Compound 38, 5.10 mg, 4.31 μmol, 6.28% yield) as a white solid. LCMS (ESI) m/z=1182.5

¹H NMR (400 MHz, METHANOL-d₄) δ 8.56-8.43 (m, 1H), 8.27 (dd, J=5.6, 15.6 Hz, 1H), 8.12 (d, J=7.6 Hz, 2H), 8.02-7.94 (m, 1H), 7.65 (t, J=8.8 Hz, 1H), 7.34 (d, J=4.0 Hz, 2H), 7.26-7.12 (m, 5H), 7.11-7.03 (m, 2H), 6.25-5.97 (m, 1H), 5.40-5.27 (m, 1H), 5.06-4.96 (m, 1H), 4.77 (d, J=10.0 Hz, 2H), 4.71-4.53 (m, 3H), 4.49-4.35 (m, 1H), 4.30-4.21 (m, 1H), 4.16-4.06 (m, 2H), 4.01-3.97 (m, 1H), 3.94-3.80 (m, 2H), 3.41 (d, J=8.4 Hz, 3H), 3.29-3.23 (m, 2H), 2.88-2.78 (m, 4H), 2.51 (td, J=7.2, 14.4 Hz, 2H), 2.35-2.28 (m, 1H), 2.21-2.08 (m, 3H), 2.05-1.93 (m, 5H), 1.91-1.84 (m, 3H), 1.70-1.59 (m, 3H), 1.55-1.44 (m, 3H), 1.32 (s, 1H), 1.23 (d, J=6.8 Hz, 3H), 0.92-0.84 (m, 3H).

### Preparation of propyl (((1S)-(2-(((3S,6S,9aS)-3-(3-(4-(4-(4-(1-(2,6-dioxopiperidin-3-yl)-3-methyl-2-oxo-2,3-dihydro-1H-benzo[d]imidazol-5-yl)but-3-yn-1-yl)piperidin-1-yl)pyridin-3-yl)azetidine-1-carbonyl)-5-oxooctahydro-1H-pyrrolo[1,2-a]azepin-6-yl)carbamoyl)benzo[b]thiophen-5-yl)fluoromethyl)(phenoxy)phosphoryl)-L-alaninate (Compound 39)

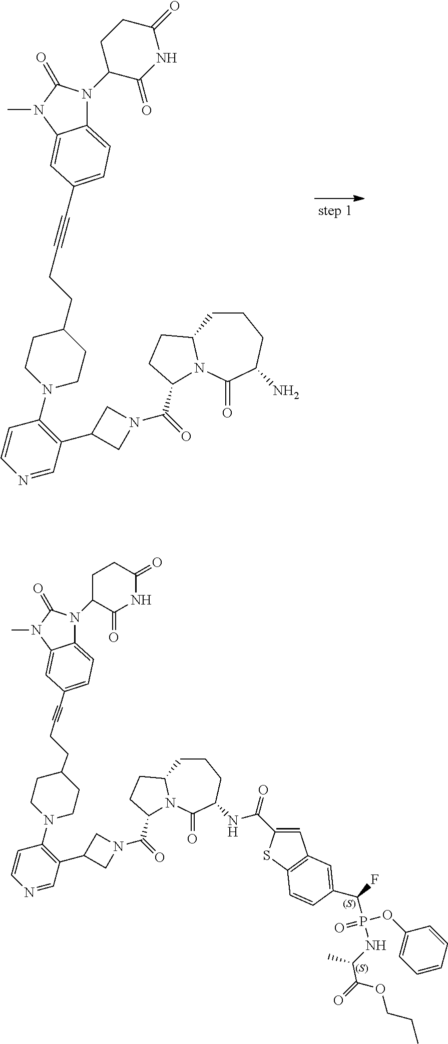

### Step 1: Preparation of propyl (((1S)-(2-(((3S,6S,9aS)-3-(3-(4-(4-(4-(1-(2,6-dioxopiperidin-3-yl)-3-methyl-2-oxo-2,3-dihydro-1H-benzo[d]imidazol-5-yl)but-3-yn-1-yl)piperidin-1-yl)pyridin-3-yl)azetidine-1-carbonyl)-5-oxooctahydro-1H-pyrrolo[1,2-a]azepin-6-yl)carbamoyl)benzo[b]thiophen-5-yl)fluoromethyl)(phenoxy)phosphoryl)-L-alaninate

To a solution of 2,3,4,5,6-pentafluorophenyl 5-[(S)-fluoro({[(2S)-1-oxo-1-propoxypropan-2-yl]amino}(phenoxy)phosphoryl)methyl]-1-benzothiophene-2-carboxylate (30 mg, 46 mol, 1 eq) in DMF (1 mL) was added 3-(5-{4-[1-(3-{1-[(3S,6S,9aS)-6-amino-5-oxo-octahydro-1H-pyrrolo[1,2-a]azepine-3-carbonyl]azetidin-3-yl}pyridin-4-yl)piperidin-4-yl]but-1-yn-1-yl}-3-methyl-2-oxo-2,3-dihydro-1H-1,3-benzodiazol-1-yl)piperidine-2,6-dione (43 mg, 60 mol, 1.3 eq) and ethylbis(propan-2-yl)amine (12 mg, 93 mol, 2 eq), the mixture was stirred at 25° C. for 10 minutes to give a yellow solution. The reaction mixture was filtered and the filtrate was purified by prep-HPLC (column: Waters xbridge 150×25 mm×10 um Waters xbridge 150×25 mm×10 um; mobile phase: [water (NH₄HCO₃)-ACN]; B %: 44%-74%, 10 min). Subsequently lyophilization afforded propyl (((1S)-(2-(((3S,6S,9aS)-3-(3-(4-(4-(4-(1-(2,6-dioxopiperidin-3-yl)-3-methyl-2-oxo-2,3-dihydro-1H-benzo[d]imidazol-5-yl)but-3-yn-1-yl)piperidin-1-yl)pyridin-3-yl)azetidine-1-carbonyl)-5-oxooctahydro-1H-pyrrolo[1,2-a]azepin-6-yl)carbamoyl)benzo[b]thiophen-5-yl)fluoromethyl)(phenoxy)phosphoryl)-L-alaninate (Compound 39, 14 mg, 12 mol, 25% yield) as a white solid. LCMS (ESI) m/z=1182.3

1H NMR (400 MHz, METHANOL-d₄) δ 8.58-8.41 (m, 1H), 8.31-8.21 (m, 1H), 8.16-8.05 (m, 2H), 8.02-7.93 (m, 1H), 7.68-7.60 (m, 1H), 7.38-7.28 (m, 2H), 7.25-7.10 (m, 5H), 7.04 (dd, J=5.6, 8.0 Hz, 2H), 6.19-5.96 (m, 1H), 5.37-5.28 (m, 1H), 4.76 (d, J=9.2 Hz, 2H), 4.70-4.62 (m, 1H), 4.61-4.52 (m, 2H), 4.49-4.33 (m, 1H), 4.30-4.20 (m, 1H), 4.16-4.04 (m, 2H), 3.98-3.94 (m, 1H), 3.94-3.85 (m, 1H), 3.39 (d, J=8.4 Hz, 3H), 3.23-3.18 (m, 2H), 2.95-2.86 (m, 1H), 2.84-2.70 (m, 4H), 2.55-2.44 (m, 2H), 2.31 (dd, J=2.8, 6.4 Hz, 1H), 2.21-2.11 (m, 2H), 2.07-1.97 (m, 3H), 1.95-1.80 (m, 6H), 1.70-1.59 (m, 3H), 1.57-1.50 (m, 2H), 1.45 (dd, J=4.4, 7.6 Hz, 2H), 1.38-1.25 (m, 2H), 1.22 (dd, J=2.4, 7.2 Hz, 2H), 0.91-0.83 (m, 3H).

### Compound 40: ((1S)-(2-(((3S,6S,9aS)-3-(3-(4-(4-(4-(1-(2,6-dioxopiperidin-3-yl)-3-methyl-2-oxo-2,3-dihydro-1H-benzo[d]imidazol-5-yl)but-3-yn-1-yl)piperidin-1-yl)pyridin-3-yl)azetidine-1-carbonyl)-5-oxooctahydro-1H-pyrrolo[1,2-a]azepin-6-yl)carbamoyl)benzo[b]thiophen-5-yl)fluoromethyl)phosphonic acid

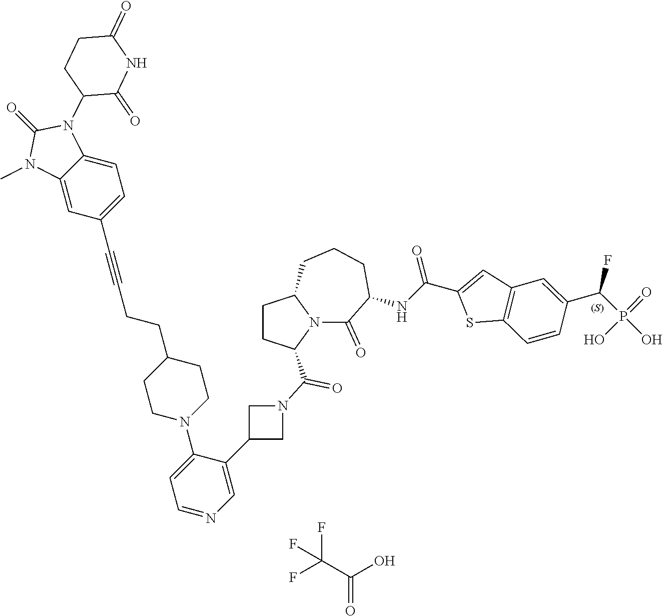

Compound 40 is the phosphoric acid version of Compound 39.

¹H NMR (400 MHz, DMSO) δ 11.11 (s, 1H), 8.76-8.66 (m, 1H), 8.51-8.25 (m, 2H), 8.21 (d, J=11.6 Hz, 1H), 8.04-7.93 (m, 2H), 7.51 (d, J=7.9 Hz, 1H), 7.27-7.16 (m, 2H), 7.12-7.05 (m, 2H), 5.79 (dd, J=44.5, 8.0 Hz, 1H), 5.41-5.34 (m, 1H), 4.79-4.65 (m, 1H), 4.45-4.35 (m, 2H), 4.29-4.24 (m, 1H), 4.15-4.09 (m, 1H), 3.98-3.93 (m, 2H), 3.60-3.56 (m, 2H), 3.35-3.31 (m, 3H), 3.08-3.00 (m, 2H), 2.92-2.84 (m, 1H), 2.76-2.58 (m, 3H), 2.47-2.43 (m, 1H), 2.24-2.14 (m, 1H), 2.09-1.64 (m, 14H), 1.60-1.48 (m, 2H), 1.37-1.20 (m, 2H). ¹⁹F NMR (377 MHz, DMSO) δ −194.50-−194.82 (m). ³¹P NMR (162 MHz, DMSO) δ 11.13-10.62 (m). (TFA salt). LCMS: Rt=1.641 min, 993.4 (M+H)+

### Biochemical and Cellular Assays

### STAT3 Fluorescence Polarization (FP) Assay

An FP assay was developed to determine IC₅₀ values for test substances.

Recombinant STAT3 protein (STAT3(G127-I722)) at 25 nM was combined with a fluorescently labeled, phosphotyrosine peptide probe (5-FAM-GpYLPQTV-NH₂) at 2 nM in FP buffer (10 mM HEPES pH 7.4, 50 mM NaCl, 1 mM EDTA, 0.05% Tween 20, 2 mM DTT). 50 μL of STAT3-probe mixture was added to serial diluted compounds in black, 96-well plates (Greiner BioOne 655076) to a final concentration of 1% DMSO. Reaction components were mixed, and FP was measured after 45-minute incubation at room temperature using a Tecan Spark multimode plate reader. FP signal (mP) was plotted against the log concentration of the test substances and IC₅₀ values were calculated by nonlinear regression analysis using GraphPad Prism software. Results are shown in Table 1. For STAT3 FP assay, A=≤100 nM; B=>100-1000 nM; C=>1-10 μM; and D=>10 μM.

### STAT6 Fluorescence Polarization (FP) Assay

An FP assay was developed to determine IC₅₀ values for test substances. Recombinant STAT6 protein (STAT6(W123-T658)) at 250 nM was combined with a fluorescently labeled, phosphotyrosine peptide probe (5-FAM-ApYKPFQDLI-NH₂) at 2 nM in FP buffer (10 mM HEPES pH 7.4, 50 mM NaCl, 1 mM EDTA, 0.05% Tween 20, 2 mM DTT). 50 μL of STAT6-probe mixture was added to serial diluted compounds in black, 96-well plates (Greiner BioOne 655076) to a final concentration of 1% DMSO. Reaction components were mixed, and FP was measured after 45-minute incubation at room temperature using a Tecan Spark multimode plate reader. FP signal (mP) was plotted against the log concentration of the test substances and IC₅₀ values were calculated by nonlinear regression analysis using GraphPad Prism software. Results are shown in Table 1. For STAT6 FP assay, A=≤300 nM; B=>300-3000 nM; C=>3-30 μM; and D=>30 μM.

TABLE 1

| Cmp# | STAT3 | STAT6 |
|---|---|---|
| 30 | A | C |
| 31 | A | C |
| 32 | A | C |
| 33 | A | C |
| 34 | A | B |
| 36 | A | D |
| 2 | A | C |
| 3 | B | A |
| 5 | C | B |
| 8 | A | B |
| 9 | A | C |
| 11 | A | C |
| 13 | B | A |
| 37 | A | C |
| 16 | B | B |
| 17 | C | A |
| 19 | C | B |
| 21 | A | C |
| 22 | B | A |
| 24 | C | B |
| 27 | C | B |
| 28 | A | C |
| 40 | A | D |

### MSD-pSTAT-PBMC Assay

### Materials:

Cryopreserved Peripheral Blood Mononuclear Cells (PBMC) are from AllCells. Recombinant Human IL-4 and IL-6 are from Peprotech. Mouse monoclonal anti-STAT3 antibody, rabbit monoclonal anti-pY705-STAT3 antibody, rabbit monoclonal Anti-pY641-STAT6 antibody, and lysis buffer are from Cell Signaling Technology (CST). Mouse monoclonal anti-STAT6 antibody is from BioLegend. Assay plates, blocker, and anti-rabbit secondary antibody are from Meso Scale Discovery (MSD).

### Assay Method:

Cryopreserved PBMCs were thawed out and allowed to recover overnight in IMDM+10% heat-inactivated FBS prior to plating 50,000 (STAT3) or 25,000 (STAT6) cells per well in 96-well U-bottom tissue culture plates. Cells were treated with compound for 3 hrs, then stimulated with 10 ng/mL IL-6 (STAT3) or 1 ng/mL IL-4 (STAT6) for 10 min. Cells were then spun down and washed with ice-cold PBS prior to lysing the cell pellet with 1× lysis buffer (CST) with 1× HALT protease and phosphatase inhibitor cocktail (Thermo). Lysates were transferred to and incubated overnight at 4° C. with shaking in QuickPlex 96-well high bind assay plates (MSD) pre-coated overnight with 30 μL per well of 0.6 pg/mL mouse monoclonal anti-STAT3 antibody (STAT3) or 2 μg/mL mouse monoclonal anti-STAT6 antibody (STAT6) in 1× PBS, and pre-blocked for 1 hour with 3% Blocker-A (MSD). Captured protein in the assay plates were then washed and probed with 25 μL per well of 0.25 pg/ml rabbit monoclonal anti-pY705-STAT3 antibody (STAT3) or 0.18 pg/ml rabbit monoclonal Anti-pY641-STAT6 antibody (STAT6) in 1% Blocker-A for 1 hour at room temperature with shaking, then washed and probed with 25 ul per well of 1 ug/ml Sulfo-TAG Labeled Goat Anti-Rabbit Antibody (MSD) in 1% Blocker-A for 1 hour at room temperature with shaking. Assay plates were then washed and 150 μL of 1× Read Buffer-T (MSD) was added to each well prior to reading on an SQ120 MSD Plate Reader. Assay signal from each sample was subtracted by the signal from unstimulated control wells and normalized to DMSO control wells. IC₅₀ values were calculated using Graph Pad Prism Dose-Response Nonlinear Regression with variable slope. Results are shown in Table 2. For both pSTAT3 and pSTAT6 A=≤100 nM; B=>100-1000 nM; C=>1000-3000 nM; and D=>3 μM.

TABLE 2

| Cmp# | pSTAT3 | pSTAT6 |
|---|---|---|
| 30 | D | |
| 31 | D | |
| 32 | D | |
| 33 | D | |
| 34 | B | |
| 35 | A | |
| 36 | B | |
| 1 | B | |
| 2 | D | |
| 8 | C | |
| 7 | B | |
| 9 | D | |
| 10 | A | |
| 11 | D | |
| 12 | A | |
| 37 | B | |
| 21 | D | |
| 20 | A | |
| 28 | D | |
| 29 | A | |
| 38 | A | |
| 39 | A | |
| 3 | | C |
| 4 | | A |
| 5 | | B |
| 6 | | A |
| 13 | | D |
| 14 | | B |
| 16 | | C |
| 15 | | A |
| 17 | | C |
| 18 | | A |
| 19 | | D |
| 22 | | C |
| 23 | | A |
| 24 | | D |
| 25 | | B |
| 27 | | D |
| 26 | | B |
| 40 | A | |

### MSD-tSTAT-PBMC Assay

### Materials:

Cryopreserved Peripheral Blood Mononuclear Cells (PBMC) for STAT3 assay are from AllCells and for STAT6 assay are from STEMCELL Technologies. Mouse monoclonal anti-STAT6 antibody is from BioLegend. The rabbit monoclonal anti-STAT6 antibody and cell lysis buffer are from Cell Signaling Technologies. Total STAT3 assay kit is from Meso Scale Discovery (MSD). Assay plates, Blocker A, Blocker D-R, Blocker D-M, and anti-rabbit secondary antibody for the total STAT6 assay is from Meso Scale Discovery (MSD).

### Assay Method:

Cryopreserved PBMCs were thawed out and allowed to recover overnight in IMDM+10% heat-inactivated FBS and 1× Glutamax prior to plating 50,000 (STAT3) or 100,000 (STAT6) cells per well in a 96-well U-bottom tissue culture plate. Cells were treated with 10 uM compound for 20 hours, then spun down and washed with ice-cold PBS prior to lysing the cell pellet with 1× lysis buffer (CST) supplemented with 1× HALT protease and phosphatase inhibitor cocktail (Thermo). Cells were then frozen at −80C, thawed, and incubated with shaking at 4C for 1 hour to allow cellular lysis. For the total STAT3 (tSTAT3) assay, assay ready plates (MSD) pre-blocked for 1 hour with 3% Blocker A (MSD) were used. Then, cell lysates were transferred to and incubated overnight at 4C with shaking in QuickPlex 96-well high bind assay plates (MSD). For the total STAT6 (tSTAT6) assay, assay plates were pre-coated overnight with 30 ul per well of 2 ug/mL mouse monoclonal anti-STAT6 antibody (BioLegend) in 1×PBS, and pre-blocked for 1 hour with 3% Blocker A (MSD). Then, cell lysates were transferred to and incubated for 72 h at 4C with shaking in QuickPlex 96-well high bind assay plates (MSD). Following protein capture on the assay plates, plates were washed and probed with appropriate antibody. For the STAT3 assay, 25 ul SULFO-tag conjugated anti-total STAT3 antibody in 1% Blocker A/0.1% Blocker D-M/0.1% Blocker D-R (MSD) was added and plates were incubated for 1 hr with shaking at room temperature. For the STAT6 assay, 25 ul per well of 0.25 ug/mL rabbit monoclonal anti-STAT6 antibody (CST) in 1% Blocker A was added and plates were incubated for 1 hour at room temperature with shaking, then washed and probed with 25 ul per well of 1 ug/mL Sulfo-TAG labeled Goat anti-Rabbit Antibody (MSD) in 1% Blocker A for 1 hour at room temperature with shaking. Assay plates were then washed and 150 ul of 1× Read Buffer-T (MSD) was added to each well prior to reading on an SQ120 MSD Plate Reader. Assay signal from each sample was subtracted by the signal from control wells and normalized to DMSO control wells to calculate percent control and percent degradation. Each condition was run in duplicate across two separate assays and values were averaged. Results are shown in Table 3. For both tSTAT3 and tSTAT6 A=>85% degradation; B=>70%-85% degradation; C=50%-70% degradation; and D=<50% degradation.

TABLE 3

| Cmp# | tSTAT3 | tSTAT6 |
|---|---|---|
| 30 | C | |
| 31 | D | |
| 32 | B | |
| 33 | D | |
| 34 | A | |
| 35 | A | |
| 36 | A | |
| 1 | C | |
| 2 | D | |
| 8 | B | |
| 7 | B | |
| 37 | A | |
| 21 | B | |
| 20 | B | |
| 3 | | A |
| 4 | | A |
| 5 | | A |
| 6 | | A |
| 13 | | B |
| 14 | | B |
| 16 | | A |
| 15 | | A |
| 17 | | A |
| 18 | | A |
| 19 | | D |
| 22 | | B |
| 23 | | A |
| 24 | | B |
| 25 | | C |
| 27 | | A |
| 26 | | B |

While we have described a number of embodiments, it is apparent that our basic examples may be altered to provide other embodiments that utilize the compounds and methods of this invention. Therefore, it will be appreciated that the scope of this invention is to be defined by the appended claims rather than by the specific embodiments that have been represented by way of example.

The contents of all references (including literature references, issued patents, published patent applications, and co-pending patent applications) cited throughout this application are hereby expressly incorporated herein in their entireties by reference. Unless otherwise defined, all technical and scientific terms used herein are accorded the meaning commonly known to one with ordinary skill in the art.

1. A compound having the structural Formula I: 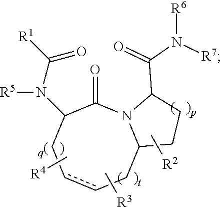
or a pharmaceutically acceptable salt thereof, wherein:
q is 0 or 1 and t is 0, 1, or 2, provided that at least one of q or t is 1;
p is 1 or 2;
the dotted line represents a single or double bond;
R¹ is selected from an 8- to 10-membered fused bicyclic heteroaryl substituted with —CR^{1a}R^{2a}P(O)OR^{1b}OR^{2b} or —CR^{1a}R^{2a}P(O)[OR^{1b}][NH(AA)C(O)OR^{T}], an 8- to 10-membered fused bicyclic heterocyclyl substituted with —CR^{1a}R^{2a}P(O)OR^{1b}OR^{2b} or —CR^{1a}R^{2a}P(O)[OR^{1b}][NH(AA)C(O)OR^{T}], an aryl substituted with CR^{1a}R^{2a}P(O)OR^{1b}OR^{2b} or —CR^{1a}R^{2a}P(O)[OR^{1b}][NH(AA)C(O)OR^{T}], a —(C₁-C₄)alkyl(aryl) wherein said aryl portion of —(C₁-C₄)alkyl(aryl) is substituted with —CR^{1a}R^{2a}P(O)OR^{1b}OR^{2b} or —CR^{1a}R^{2a}P(O)[OR^{1b}][NH(AA)C(O)OR^{T}], and a —(C₂-C₄)alkenyl(aryl) wherein said aryl portion of —(C₂-C₄)alkenyl(aryl) is substituted with —CR^{1a}R^{2a}P(O)OR^{1b}OR^{2b} or —CR^{1a}R^{2a}P(O)[OR^{1b}][NH(AA)C(O)OR^{T}];
R^{1a} and R^{2a} are each absent or are independently selected from hydrogen, cyano, (C₁-C₄)alkyl, hydroxy(C₁-C₄)alkyl and fluoro; or R^{1a} and R^{2a} taken together with the carbon they are attached form oxo;
R^{1b} and R^{2b} are each absent or are independently selected from hydrogen, (C₁-C₄)alkyl, halo(C₁-C₄)alkyl, —[(C₁-C₄)alkyl]-OC(O)—[(C₁-C₄)alkyl], —[(C₁-C₄)alkyl]-C(O)O—[(C₁-C₄)alkyl], —[(C₁-C₄)alkyl]-O—[(C₁-C₂₀)alkyl], —[(C₁-C₄)alkyl]-OC(O)—[halo(C₁-C₄)alkyl], [(C₁-C₄)alkyl]-OC(O)O—[5- to 7-membered heterocyclyl], [(C₁-C₄)alkyl]-OC(O)—[5- to 7-membered heterocyclyl], —[(C₁-C₄)alkyl]-OC(O)—[(C₁-C₄)alkyl]-OH, —[(C₁-C₄)alkyl]-OC(O)—[(C₁-C₄)alkyl]-O—[(C₁-C₄)alkyl], —[(C₁-C₄)alkyl]-OC(O)O—[(C₁-C₄)alkyl], —[(C₁-C₄)alkyl]-OC(O)O—[halo(C₁-C₄)alkyl], —[(C₁-C₄)alkyl]-OC(O)O—[(C₁-C₄)alkyl]-OH, —[(C₁-C₄)alkyl]-OC(O)O—[(C₁-C₄)alkyl]-O—[(C₁-C₄)alkyl], —[(C₁-C₄)alkyl]-SC(O)—[(C₁-C₄)alkyl], —[(C₁—C₄)alkyl]-SC(O)—[halo(C₁-C₄)alkyl], —[(C₁-C₄)alkyl]-SC(O)—[(C₁-C₄)alkyl]-OH, —[(C₁-C₄)alkyl]-SC(O)—[(C₁-C₄)alkyl]-O—[(C₁-C₄)alkyl], —[(C₁-C₄)alkyl]-OC(O)NH(C₁-C₄)alkyl], —[(C₁-C₄)alkyl]-OC(O)N[(C₁-C₄)alkyl]2, 5- to 6-membered heteroaryl, and aryl, wherein said 5- to 6-membered heteroaryl and aryl are each optionally and independently substituted with, as valency permits, 1 to 2 groups selected from halo, cyano, and (C₁-C₄)alkyl and wherein said 5- to 7-membered heterocyclyl of [(C₁-C₄)alkyl]-OC(O)O—[5- to 7-membered heterocyclyl] and [(C₁-C₄)alkyl]-OC(O)—[5- to 7-membered heterocyclyl] are each optionally and independently substituted with, as valency permits 1 to 2 groups selected from C(O)OR^{h};
R² is selected from hydrogen, halo, (C₁-C₄)alkyl, halo(C₁-C₄)alkyl, (C₁-C₄)alkoxy, halo(C₁-C₄)alkoxy, hydroxy(C₁-C₄)alkyl, cyano, and hydroxyl;
R³ and R⁴ are each independently selected from hydrogen, halo, (C₁-C₄)alkyl, halo(C₁-C₄)alkyl, hydroxy(C₁-C₄)alkyl, —(C₁-C₄)alkylphenyl, (C₁-C₄)alkoxy, halo(C₁-C₄)alkoxy, —(C₁-C₄)alkyl(C₁-C₄)alkoxy, hydroxyl, cyano, —NR^{a}R^{b}, phenyl, (C₃-C₆)cycloalkyl, 5- to 6-membered heteroaryl, and 4- to 6-membered heterocyclyl, wherein said phenyl, (C₃-C₆)cycloalkyl, 5- to 6-membered heteroaryl, and 4- to 6-membered heterocyclyl are each optionally substituted with, as valency permits, 1 to 3 groups selected from R^{S};
or R³ and R⁴ are taken together on the same carbon atom to form a (C₃-C₆)cycloalkyl or a 4- to 6-membered heterocyclyl each optionally substituted with, as valency permits, 1 to 3 groups selected from halo, (C₁-C₄)alkyl, halo(C₁-C₄)alkyl, (C₁-C₄)alkoxy, and halo(C₁-C₄)alkoxy;
R⁵ and R⁶ are each independently selected from hydrogen and (C₁-C₄)alkyl;
R⁷ is selected from E, -R^{10A}E, (C₁-C₄)alkyl, phenyl, 4- to 9-membered monocyclic or bicyclic heterocyclyl, and 5- to 10-membered monocyclic or bicyclic heteroaryl, wherein said (C₁-C₄)alkyl is optionally substituted with, as valency permits, 1 to 3 groups selected from R^{Y} and said phenyl, 4- to 9-membered monocyclic or bicyclic heterocyclyl, and 5- to 10-membered monocyclic or bicyclic heteroaryl are each optionally substituted with, as valency permits, R¹¹ and/or 1 to 3 groups selected from R^{Z}; or
R⁶ and R⁷ together with the nitrogen atom to which they are attached form a 4- to 14-membered monocyclic or bicyclic heterocyclyl or a 5- to 12-membered monocyclic or bicyclic heteroaryl, each of which being optionally substituted with, as valency permits, R¹² and/or 1 to 3 groups selected from R^{Q};
R^{10A}, R^{10B}, R^{10C}, and R^{10D} are each independently a chemical spacer unit;
R¹¹ is selected from —NHC(O)R^{10B}E, —OR^{10B}E, phenyl, (C₃-C₆)cycloalkyl, 5- to 6-membered heteroaryl, and 4- to 6-membered heterocyclyl each of said phenyl, (C₃-C₆)cycloalkyl, 5- to 6-membered heteroaryl, and 4- to 6-membered heterocyclyl being substituted with —OR^{10B}E, —NHR^{10B}E, —[N(C₁-C₄)alkyl]R^{10B}E, or R^{10B}E, and wherein said 4- to 6-membered heterocyclyl is optionally substituted further with oxo;
R¹² is selected from —NHC(O)R^{10C}E, —OR^{10C}E, phenyl, (C₃-C₆)cycloalkyl, 5- to 6-membered heteroaryl, and 4- to 6-membered heterocyclyl each of said phenyl, (C₃-C₆)cycloalkyl, 5- to 6-membered heteroaryl, and 4- to 6-membered heterocyclyl being substituted with —OR^{10C}E, —NHR^{10C}E, —[N(C₁-C₄)alkyl]R^{10C}E, or R^{10C}E, and wherein said 4- to 6-membered heterocyclyl is optionally substituted further with oxo;
R¹³ is selected from —NHC(O)R^{10D}E, —OR^{10D}E, phenyl, (C₃-C₆)cycloalkyl, 5- to 6-membered heteroaryl, and 4- to 6-membered heterocyclyl each of said phenyl, (C₃-C₆)cycloalkyl, 5- to 6-membered heteroaryl, and 4- to 6-membered heterocyclyl being substituted with —OR^{10D}E, —NHR^{10D}E, —[N(C₁-C₄)alkyl]R^{10D}E, or R^{10D}E, and wherein said 4- to 6-membered heterocyclyl is optionally substituted further with oxo;
E is a chemical moiety that binds to E3 ligase;
AA is the residue of an alpha or beta natural or non-natural amino acid;
R^{T} is selected from (C₁-C₄)alkyl, benzyl, and phenyl, wherein said phenyl is optionally substituted with 1 or 2 groups selected from halo, (C₁-C₄)alkyl, and halo(C₁-C₄)alkyl;
R^{Q} is selected from halo, (C₂-C₄)alkenyl, (C₁-C₄)alkyl, (C₁-C₄)alkoxy, halo(C₁-C₄)alkoxy, cyano, phenyl, hydroxyl, 4- to 6-membered heterocyclyl, 5- to 10-membered monocyclic or bicyclic heteroaryl, (C₃-C₆)cycloalkyl, oxo, imino, —O(phenyl), —C(O)R^{g}, —C(O)OR^{e}, —NHC(O)R^{e}, —C(O)NR^{c}R^{d}, —NR^{a}R^{b}, —S(O)R^{e}R^{f}, —S(O)₂R^{f}, —S(O)═NH(C₁-C₄)alkyl, —S(O)NR^{e}R^{f}, and —S(O)₂NR^{e}R^{f}, wherein said (C₂-C₄)alkenyl and (C₁-C₄)alkyl are each optionally and independently substituted with, as valency permits, 1 to 3 groups selected from R^{M}, and wherein said phenyl, 5- to 10-membered monocyclic or bicyclic heteroaryl, (C₃-C₆)cycloalkyl, and 4- to 6-membered heterocyclyl are each optionally and independently substituted with, as valency permits, 1 to 3 groups selected from R^{F};
R^{Y} is selected from halo, (C₁-C₄)alkoxy, halo(C₁-C₄)alkoxy, cyano, —C(O)R^{g}, —C(O)OR^{e}, —NHC(O)R^{e}, —NR^{a}R^{b}, —S(O)R^{e}R^{f}, —S(O)₂R^{f}, —S(O)NR^{e}R^{f}, —S(O)═NH(C₁-C₄)alkyl, —S(O)₂NR^{e}R^{f}, hydroxyl, phenyl, 4- to 6-membered heterocyclyl, and 5- to 10-membered monocyclic or bicyclic heteroaryl, wherein said phenyl, 4- to 6-membered heterocyclyl, and 5- to 10-membered monocyclic or bicyclic heteroaryl are each optionally substituted with, as valency permits, 1 to 3 groups selected from R^{X};
R^{J} and R^{M} are each independently selected from halo, (C₁-C₄)alkoxy, halo(C₁-C₄)alkoxy, cyano, —C(O)R^{g}, —C(O)OR^{e}, —NHC(O)R^{e}, —C(O)NR^{c}R^{d}, —NR^{a}R^{b}, —S(O)R^{e}R^{f}, —S(O)₂R, —S(O)NR^{e}R^{f}, —S(O)═NH(C₁-C₄)alkyl, —S(O)₂NR^{e}R^{f}, hydroxyl, phenyl, 4- to 6-membered heterocyclyl, and 5- to 10-membered monocyclic or bicyclic heteroaryl, wherein said phenyl, 4- to 6-membered heterocyclyl, and 5- to 10-membered monocyclic or bicyclic heteroaryl are each optionally substituted with, as valency permits, 1 to 3 groups selected from R^{X};
R^{F}, R^{S}, and R^{X} are each independently selected from halo, cyano, (C₁-C₄)alkyl, halo(C₁-C₄)alkyl, —(C₁-C₄)alkyl(C₁-C₄)alkoxy, hydroxy(C₁-C₄)alkyl, —(C₁-C₄)alkylphenyl, (C₂-C₄)alkenyl, halo(C₂-C₄)alkenyl, (C₂-C₄)alkynyl, halo(C₂-C₄)alkynyl, (C₁-C₄)alkoxy, halo(C₁-C₄)alkoxy, hydroxyl, oxo, imino, phenyl, —S(O)R^{e}R^{f}, —S(O)₂R^{f}, —S(O)═NH(C₁-C₄)alkyl, —S(O)NR^{e}R^{f}, and —S(O)₂NR^{e}R^{f}, —C(O)OR^{e}, —NR^{c}C(O)R^{e}, —C(O)R^{g}, —C(O)NR^{c}R^{d}, and —NR^{a}R^{b}, wherein said phenyl and said phenyl for the group —(C₁-C₄)alkylphenyl are each optionally and independently substituted with, as valency permits 1 to 3 groups selected from halo, cyano, (C₁-C₁₀)alkyl, (C₂-C₁₀)alkenyl, (C₂-C₁₀)alkynyl, halo(C₁-C₁₀)alkyl, (C₁-C₁₀)alkoxy, and halo(C₁-C₁₀)alkoxy, wherein said (C₁-C₁₀)alkyl, (C₂-C₁₀)alkenyl and (C₂-C₁₀)alkynyl are each optionally substituted with, as valency permits a 5- to 10-membered monocyclic or bicyclic heteroaryl or a 4- to 10-membered monocyclic or bicyclic heterocyclyl each of said 5- to 10-membered monocyclic and bicyclic heteroaryl or a 4- to 10-membered monocyclic or bicyclic heterocyclyl being optionally substituted with oxo or a 5- to 7-membered heterocyclyl that is optionally substituted with 1 to 2 oxo;
R^{Z} is selected from halo, cyano, (C₁-C₄)alkyl, (C₃-C₆cycloalkyl), halo(C₁-C₄)alkyl, —(C₁-C₄)alkyl(C₁-C₄)alkoxy, hydroxy(C₁-C₄)alkyl, —(C₁-C₄)alkylphenyl, —(C₁-C₄)alkylheteroaryl, (C₂-C₄)alkenyl, halo(C₂-C₄)alkenyl, (C₂-C₄)alkynyl, halo(C₂-C₄)alkynyl, (C₁-C₄)alkoxy, halo(C₁-C₄)alkoxy, —OR^{e}, oxo, imino, phenyl, 4- to 6-membered heterocyclyl, —S(O)R^{e}R^{f}, —S(O)₂R^{f}, —S(O)═NH(C₁-C₄)alkyl, —S(O)NR^{e}R^{f}, and —S(O)₂NR^{e}R^{f}, —C(O)OR^{e}, —NR^{c}C(O)R^{e}, —(C₁-C₄alkyl)C(O)R^{g}, —C(O)R^{g}, —(C₁-C₄alkyl)C(O)NR^{c}R^{d}, —C(O)NR^{c}R^{d}, —NO₂, and —NR^{a}R^{b}, wherein the (C₁-C₄)alkyl is optionally substituted with cyano, wherein said phenyl, said 4- to 6-membered heterocyclyl, and said phenyl for —(C₁-C₄)alkylphenyl are each optionally and independently substituted with, as valency permits R¹³ and/or 1 to 3 groups selected from halo, cyano, oxo, (C₁-C₁₀)alkyl, (C₂-C₁₀)alkenyl, (C₂-C₁₀)alkynyl, halo(C₁—C₁₀)alkyl, (C₁-C₁₀)alkoxy, and halo(C₁-C₁₀)alkoxy, wherein said (C₁-C₁₀)alkyl, (C₂-C₁₀)alkenyl and (C₂-C₁₀)alkynyl are each optionally substituted with, as valency permits a 5- to 10-membered monocyclic or bicyclic heteroaryl or a 4- to 10-membered monocyclic or bicyclic heterocyclyl each of said 5- to 10-membered monocyclic and bicyclic heteroaryl or a 4- to 10-membered monocyclic or bicyclic heterocyclyl being optionally substituted with oxo or a 5- to 7-membered heterocyclyl that is optionally substituted with 1 to 2 oxo;
R^{a}, R^{b}, R^{c}, R^{d}, R^{e}, R^{f}, R^{g}, and R^{h} are each independently selected from, as valency permits, hydrogen, (C₁-C₄)alkyl, phenyl, (C₃-C₆)cycloalkyl, 4- to 6-membered heterocyclyl and 5- to 6-membered heteroaryl, wherein said (C₁-C₄)alkyl is optionally substituted with, as valency permits, 1 to 3 groups selected from R^{J}, and said phenyl, (C₃-C₆)cycloalkyl, 4- to 6-membered heterocyclyl, and 5- to 6-membered heteroaryl are each independently optionally substituted with, as valency permits, 1 to 3 groups selected from halo, cyano, (C₁-C₄)alkyl, halo(C₁-C₄)alkyl, (C₁-C₄)alkoxy, halo(C₁-C₄)alkoxy, hydroxyl, phenyl, and benzyl.

2. The compound of claim 1, or a pharmaceutically acceptable salt thereof, wherein, R⁶ and R⁷ together with the nitrogen atom to which they are attached form a 4- to 14-membered monocyclic or bicyclic heterocyclyl or a 5- to 12-membered monocyclic or bicyclic heteroaryl, each of which being optionally substituted with R¹² and/or, as valency permits, optionally further substituted with 1 to 3 groups selected from R^{Q}.

3. The compound of claim 1 or 2, or pharmaceutically acceptable salt thereof, wherein the compound is of the structural Formula II:

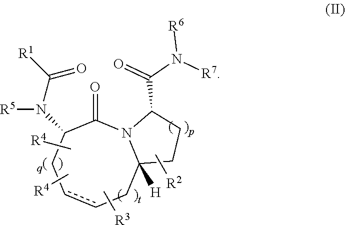

4. The compound of any one of claims 1 to 3, or pharmaceutically acceptable salt thereof, wherein the compound is of the structural III, IV, V, or VII:

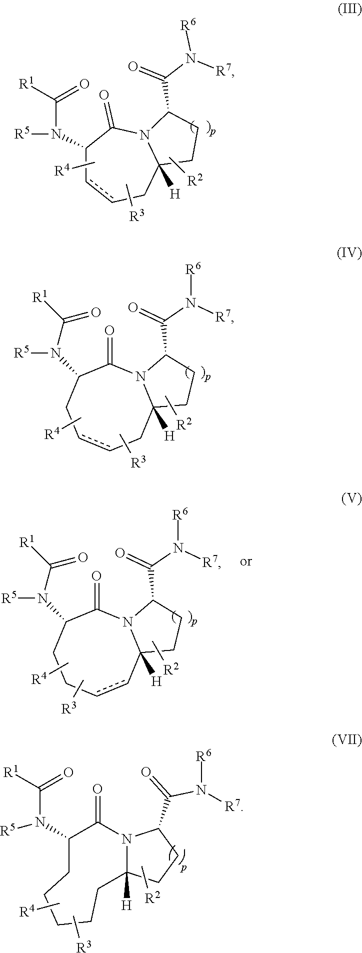

5. The compound of any one of claims 1 to 4, or pharmaceutically acceptable salt thereof, wherein the compound is of the structural VIII, VIII′, IX, X, XI, XII, or XIII:

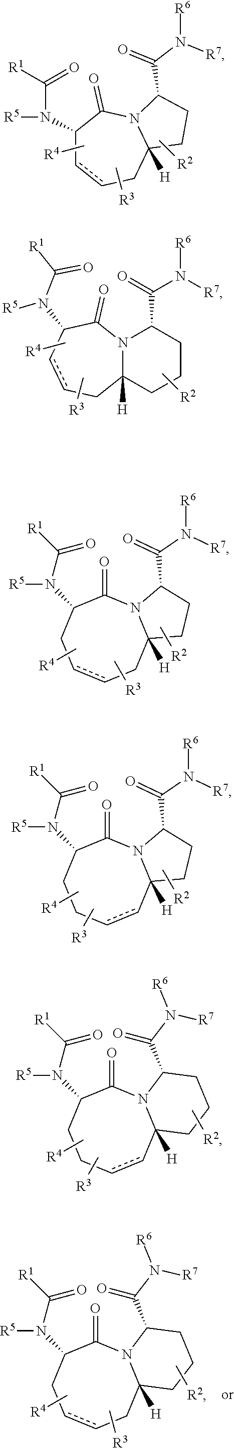

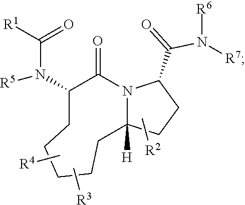

6. The compound of any one of claims 1 to 5, or pharmaceutically acceptable salt thereof, wherein the compound is of the structural XIV, XV, XVI, XVI′, XVII, XVIII, XX, XXI, XXII, XXIII, XXIV, XXV, XXVI, XXVII, or XXVIII:

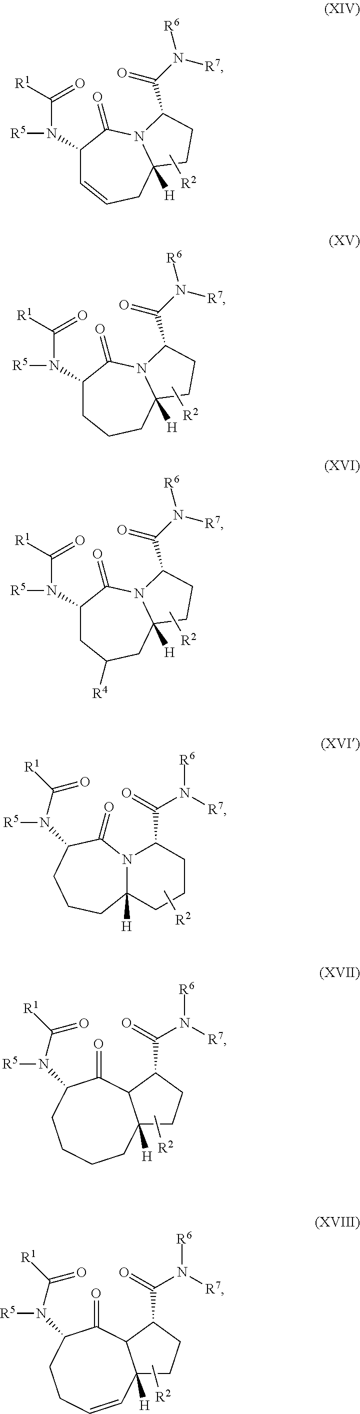

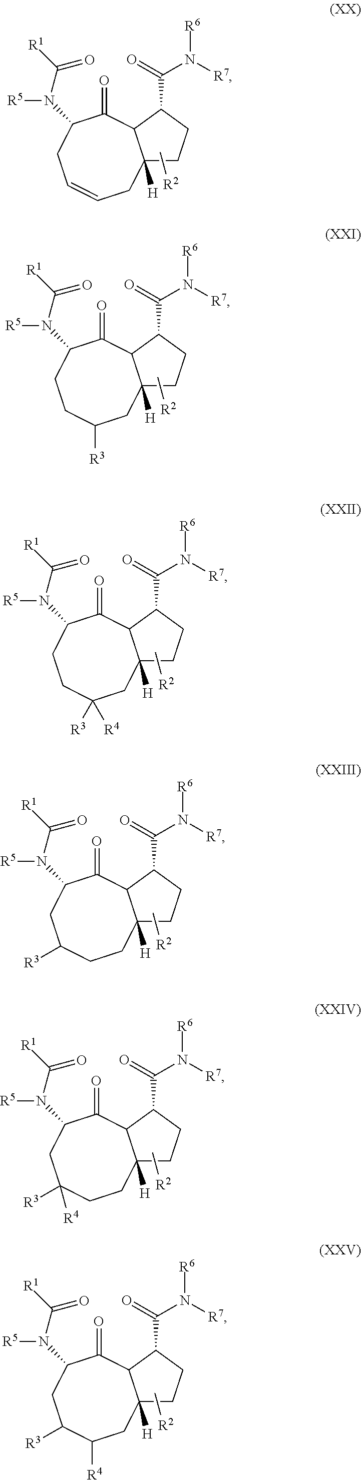

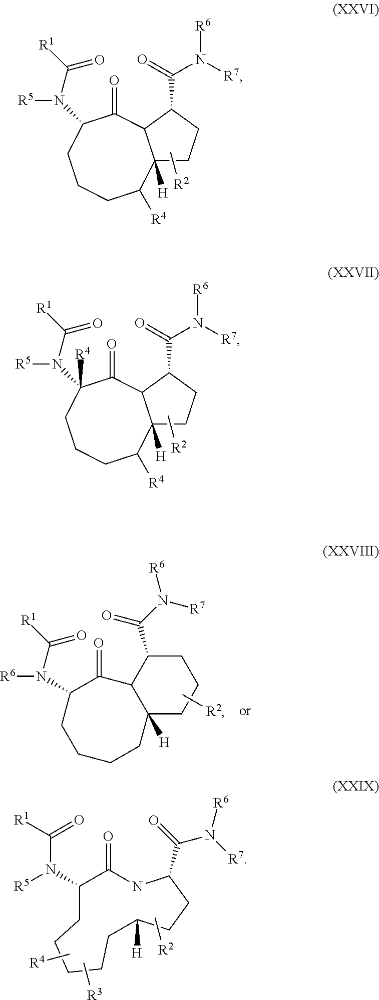

7. The compound of any one of claims 1 to 6, or pharmaceutically acceptable salt thereof, wherein the compound is of the structural Formula XXX, XXXI, XXXII, XXXIII XXXIV, XXXV, XXXVI, or XXVII:

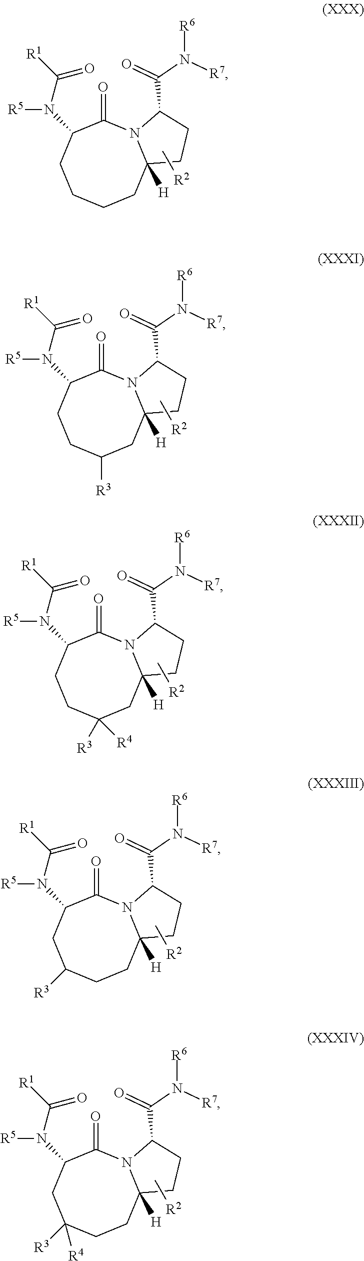

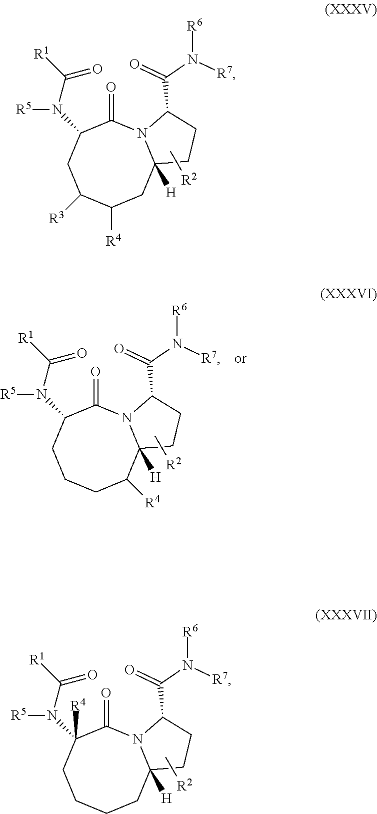

8. The compound of any one of claims 1 to 7, or pharmaceutically acceptable salt thereof, wherein R³ is selected from hydrogen, (C₁-C₄)alkyl, hydroxyl, (C₁-C₄)alkoxy, —(C₁-C₄)alkylphenyl, and 4- to 6-membered heterocyclyl; or R³ and R⁴ are taken together on the same carbon atom to form a (C₃-C₆)cycloalkyl.

9. The compound of any one of claims 1 to 8, or pharmaceutically acceptable salt thereof, wherein R⁴ is selected from hydrogen (C₁-C₄)alkyl, and hydroxyl; or R³ and R⁴ are taken together on the same carbon atom to form a (C₃-C₆)cycloalkyl.

10. The compound of any one of claims 1 to 9, or pharmaceutically acceptable salt thereof, wherein R³ is selected from hydrogen, (C₁-C₂)alkyl, hydroxyl, (C₁-C₂)alkoxy, benzyl, and azetidinyl; or R³ and R⁴ are taken together on the same carbon atom to form a cyclopropyl.

11. The compound of any one of claims 1 to 10, or pharmaceutically acceptable salt thereof, wherein R³ and R⁴ are hydrogen.

12. The compound of any one of claims 1 to 11, or pharmaceutically acceptable salt thereof, wherein R² is selected from hydrogen and hydroxyl.

13. The compound of any one of claims 1 to 12, or pharmaceutically acceptable salt thereof, wherein R² is hydrogen.

14. The compound of any one of claims 1 to 13, or pharmaceutically acceptable salt thereof, wherein R⁵ is hydrogen.

15. The compound of any one of claims 1 to 14, or pharmaceutically acceptable salt thereof, wherein R¹ is selected from 8- to 10-membered fused bicyclic heteroaryl and aryl, each of which are substituted with —CR^{1a}R^{2a}P(O)OR^{1b}OR^{2b} or —CR^{1a}R^{2a}P(O)[OR^{1b}][NH(AA)C(O)OR^{T}].

16. The compound of any one of claims 1 to 15, or pharmaceutically acceptable salt thereof, wherein, R¹ is selected from benzothiophenyl, indolyl, and naphthalenyl, each of which are substituted with —CR^{1a}R^{2a}P(O)OR^{1b}OR^{2b} or —CR^{1a}R^{2a}P(O)[OR^{1b}][NH(AA)C(O)OR^{T}].

17. The compound of any one of claims 1 to 16, or pharmaceutically acceptable salt thereof, wherein, R¹ is selected from,

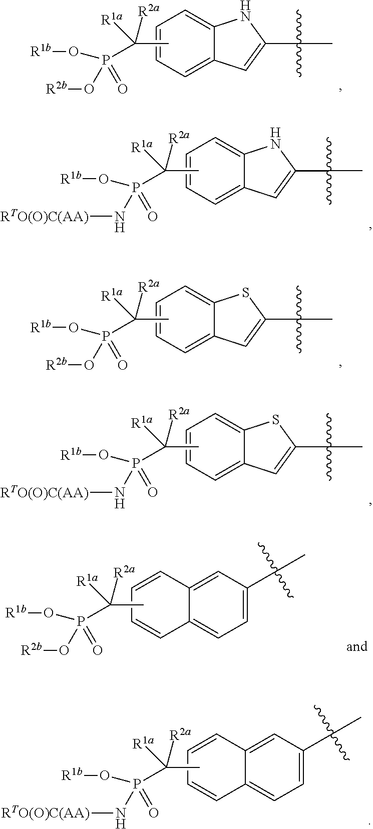

18. The compound of any one of claims 1 to 17, or pharmaceutically acceptable salt thereof, wherein, R¹ is

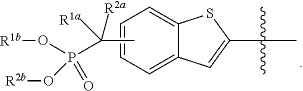

19. The compound of any one of claims 1 to 18, or pharmaceutically acceptable salt thereof, wherein R^{1a} is hydrogen and R^{2a} is fluoro or R^{1a} is fluoro and R^{2a} is fluoro.

20. The compound of any one of claims 1 to 19, or pharmaceutically acceptable salt thereof, wherein R^{1a} and R^{2a} are fluoro.

21. The compound of any one of claims 1 to 20, or pharmaceutically acceptable salt thereof, wherein R^{1b} and R^{2b} are each independently selected from hydrogen, (C₁-C₄)alkyl, —[(C₁-C₄)alkyl]-OC(O)—[(C₁-C₄)alkyl], —[(C₁-C₄)alkyl]-OC(O)O—[(C₁-C₄)alkyl], —[(C₁-C₄)alkyl]-SC(O)—[(C₁-C₄)alkyl], —[(C₁-C₄)alkyl]-SC(O)—[halo(C₁-C₄)alkyl], —[(C₁-C₄)alkyl]-SC(O)—[(C₁-C₄)alkyl]-OH, phenyl, pyridinyl, and naphthalenyl, wherein said phenyl, pyridinyl, and naphthalenyl are each optionally and independently substituted with cyano.

22. The compound of any one of claims 1 to 21, or pharmaceutically acceptable salt thereof, wherein R^{1b} and R^{2b} are each —[(C₁-C₄)alkyl]-OC(O)—[(C₁-C₄)alkyl].

23. The compound of any one of claims 1 to 21, or pharmaceutically acceptable salt thereof, wherein R^{1b} and R^{2b} are hydrogen.

24. The compound of any one of claims 1 to 18 and 21, or pharmaceutically acceptable salt thereof, wherein —CR^{1a}R^{2a}P(O)OR^{1b}OR^{2b} is selected from

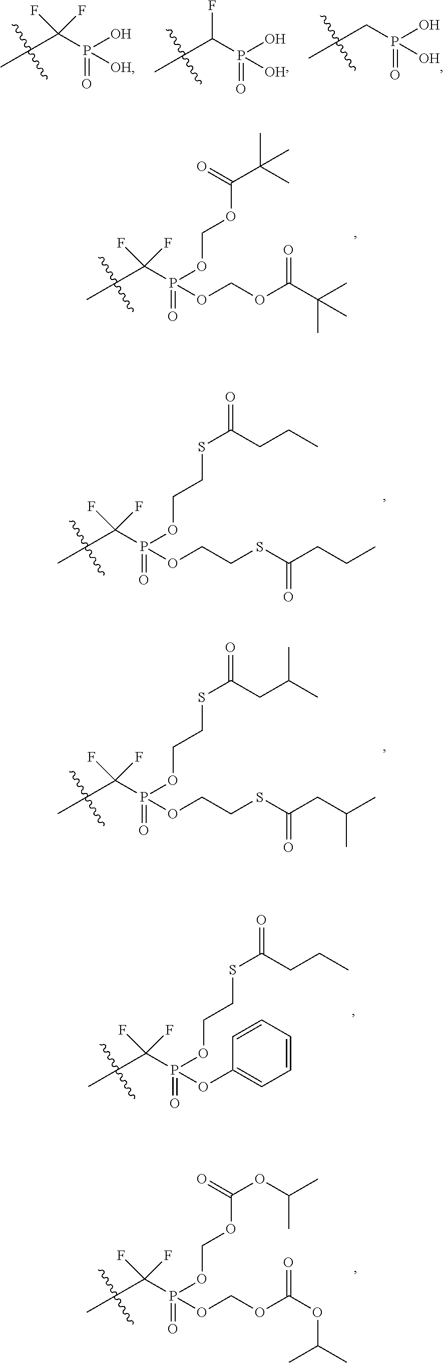

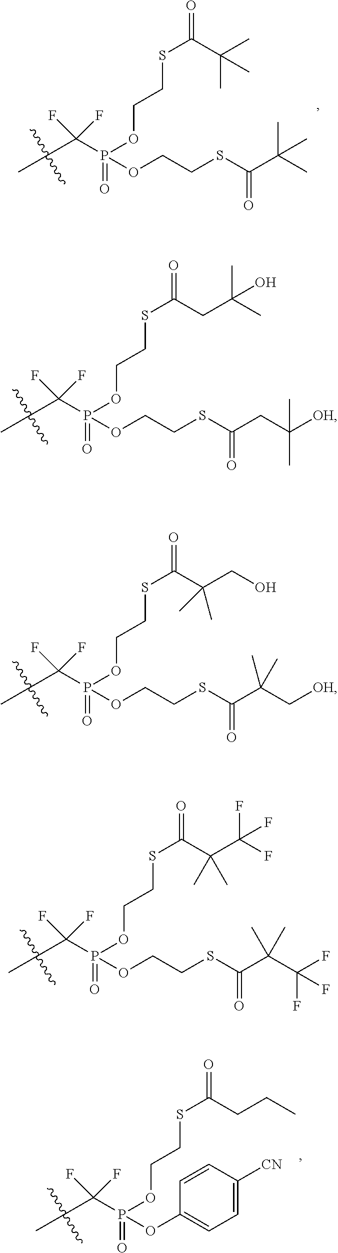 [SMILES: *C(F)(F)P(=O)(OCCSC(=O)C(C)(C)C)OCCSC(=O)C(C)(C)C ; *C(F)(F)P(=O)(OCCSC(=O)CC(C)(C)O)OCCSC(=O)CC(C)(C)O ; *C(F)(F)P(=O)(OCCSC(=O)C(C)(C)CO)OCCSC(=O)C(C)(C)CO ; *C(F)(F)P(=O)(OCCSC(=O)C(C)(C)C(F)(F)F)OCCSC(=O)C(C)(C)C(F)(F)F ; *C(F)(F)P(=O)(OCCSC(=O)CCC)Oc1ccc(C#N)cc1]

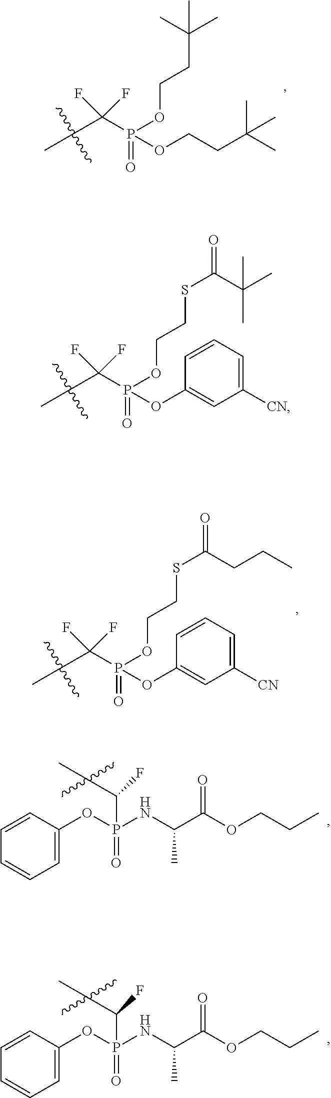 [SMILES: *C(F)(F)P(=O)(OCCC(C)(C)C)OCCC(C)(C)C ; *C(F)(F)P(=O)(OCCSC(=O)C(C)(C)C)Oc1cccc(C#N)c1 ; *C(F)(F)P(=O)(OCCSC(=O)CCC)Oc1cccc(C#N)c1 ; *[C@H](F)P(=O)(N[C@@H](C)C(=O)OCCC)Oc1ccccc1 ; *[C@@H](F)P(=O)(N[C@@H](C)C(=O)OCCC)Oc1ccccc1]

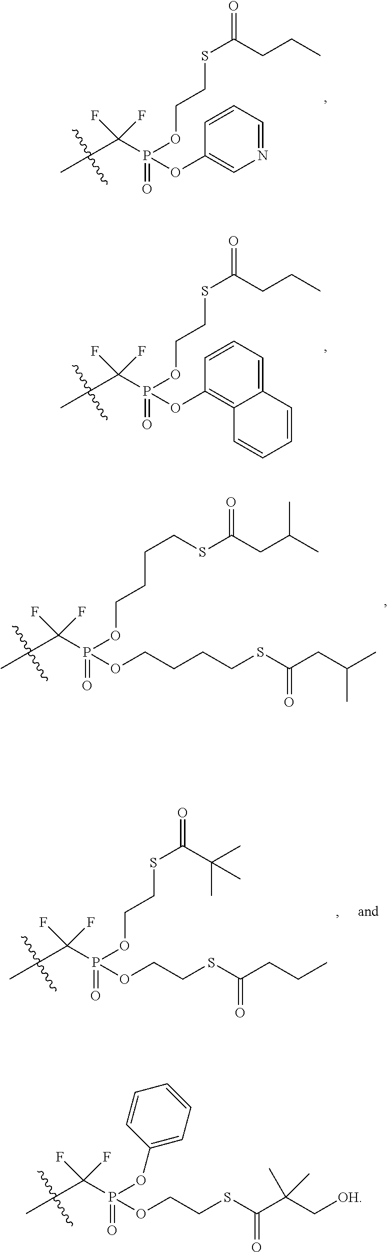 [SMILES: *C(F)(F)P(=O)(OCCSC(=O)CCC)Oc1cccnc1 ; *C(F)(F)P(=O)(OCCSC(=O)CCC)Oc1cccc2ccccc12 ; *C(F)(F)P(=O)(OCCCCSC(=O)CC(C)C)OCCCCSC(=O)CC(C)C ; *C(F)(F)P(=O)(OCCSC(=O)CCC)OCCSC(=O)C(C)(C)C ; *C(F)(F)P(=O)(OCCSC(=O)C(C)(C)CO)Oc1ccccc1]

25. The compound of any one of claims 1 to 18 and 21, or pharmaceutically acceptable salt thereof, wherein —CR^{1a}R^{2a}P(O)OR^{1b}OR^{2b} is selected from

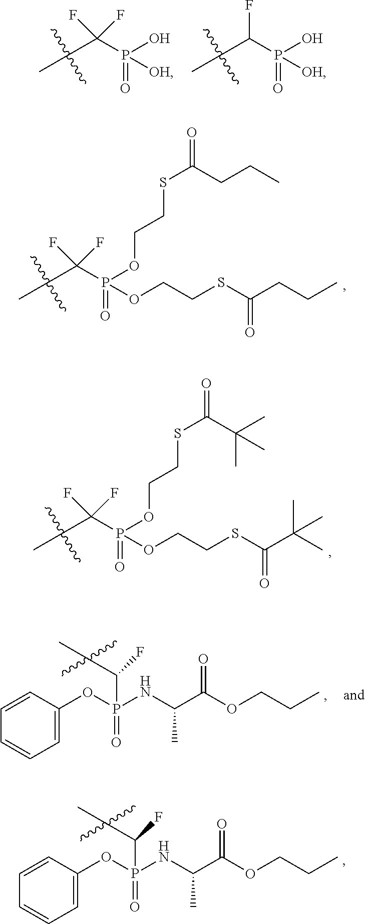 [SMILES: *C(F)(F)P(=O)(O)O ; *C(F)P(=O)(O)O ; *C(F)(F)P(=O)(OCCSC(=O)CCC)OCCSC(=O)CCC ; *C(F)(F)P(=O)(OCCSC(=O)C(C)(C)C)OCCSC(=O)C(C)(C)C ; *[C@H](F)P(=O)(N[C@@H](C)C(=O)OCCC)Oc1ccccc1 ; *[C@@H](F)P(=O)(N[C@@H](C)C(=O)OCCC)Oc1ccccc1]

26. The compound of any one of claims 1 to 25, or pharmaceutically acceptable salt thereof, wherein R⁶ and R⁷ together with the nitrogen atom to which they are attached form a 4- to 9-membered monocyclic or bicyclic heterocyclyl substituted with R¹² and/or, as valency permits, optionally further substituted with 1 to 3 groups selected from R^{Q}.

27. The compound of any one of claims 1 to 26, or pharmaceutically acceptable salt thereof, wherein R⁶ and R⁷ together with the nitrogen atom to which they are attached form pyrrolidinyl, azetidinyl, or 4-azaspiro[2.4]heptanyl, each substituted with R¹² and/or, as valency permits, optionally further substituted with 1 to 3 groups selected from R^{Q}.

28. The compound of any one of claims 1 to 27, or pharmaceutically acceptable salt thereof, wherein R^{Q} is cyano or 5- to 7-membered heteroaryl.

29. The compound of any one of claims 1 to 28, or pharmaceutically acceptable salt thereof, wherein R^{Q} is cyano or pyridinyl.

30. The compound of any one of claims 1 to 29, or pharmaceutically acceptable salt thereof, wherein R¹² is selected from —NHC(O)R^{10C}E, —OR^{10C}E, phenyl, pyridinyl, and pyridin-2(1H)-one, each substituted with R^{10C}E, —OR^{10C}E, —NH R^{10C}E, or —[N(C₁-C₄)alkyl]R^{10C}E.

31. The compound of any one of claims 1 to 30, or pharmaceutically acceptable salt thereof, wherein R^{10C} is selected from (C₁-C₁₀)alkylene and (C₂-C₁₀)alkynelene each optionally substituted with one or more groups selected from halo and oxo, and wherein said (C₁-C₆)alkylene and (C₂-C₁₀)alkynelene may also be optionally interrupted by one or more heteroatoms selected from S, N, and O, and/or optionally interrupted by one or more rings selected from 5- to 7-membered heteroaryl, (C₃-C₆)cycloakyl, phenyl, and 4- to 7-membered heterocyclyl.

32. The compound of any one of claims 1 to 31, or pharmaceutically acceptable salt thereof, wherein R^{10C} is selected from (C₁-C₁₀)alkylene, (C₂-C₁₀)alkynelene, —(CH₂)ₘC(O)NH[(CH₂)O]ᵥ(CH₂)ₙ, —(CH₂)O[(CH₂)O]ᵥ(CH₂)ₙ, —(C₂-C₈)alkynelene[4- to 7-membered heterocyclyl], 4- to 7-membered heterocyclyl, [(CH₂)O]ᵥ(CH₂)ₙ, and —(CH₂)ₙC(O)NH[4- to 7-membered heterocyclyl], wherein m, n, and v are each independently selected from 0, 1, 2, 3, 4, 5, and 6.

33. The compound of any one of claims 1 to 32, or pharmaceutically acceptable salt thereof, wherein R^{10C} is selected from: 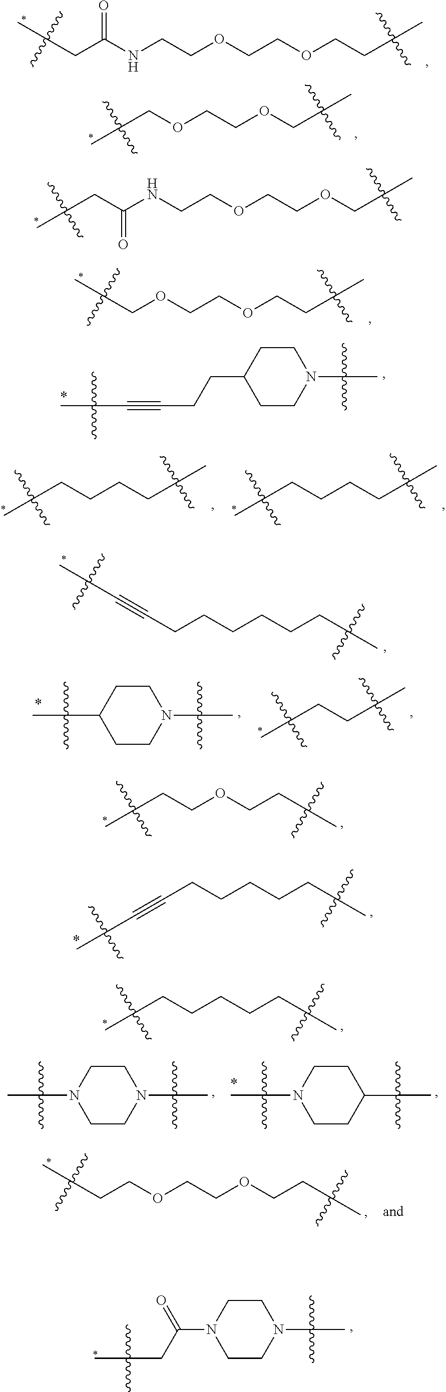
wherein the asterisk indicates the position to which E is attached.

34. The compound of any one of claims 1 to 33, or pharmaceutically acceptable salt thereof, wherein E is selected from a CRBN, VHL, IAP, or MDM2 based E3 binder.

35. The compound of any one of claims 1 to 34, or pharmaceutically acceptable salt thereof, wherein E is selected from 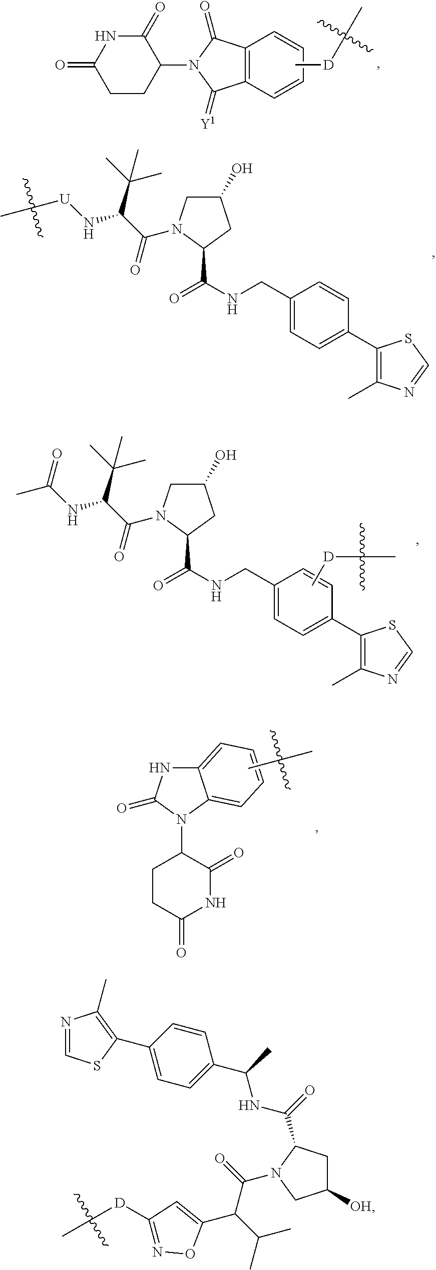 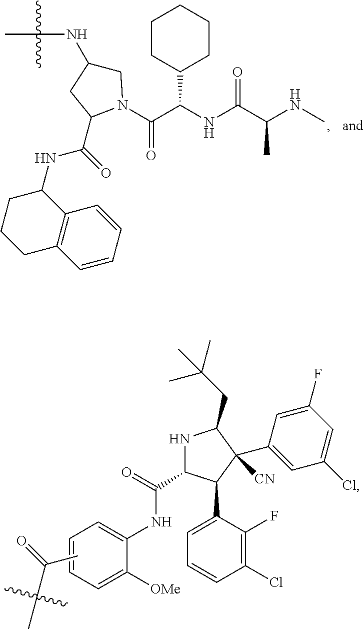
wherein:
Y and Y¹ are each independently is O or CH₂;
D is O, NH, or a bond;
U is absent or C(O); and
R is halo or cyano.

36. The compound of any one of claims 1 to 35, or pharmaceutically acceptable salt thereof, wherein E is selected from

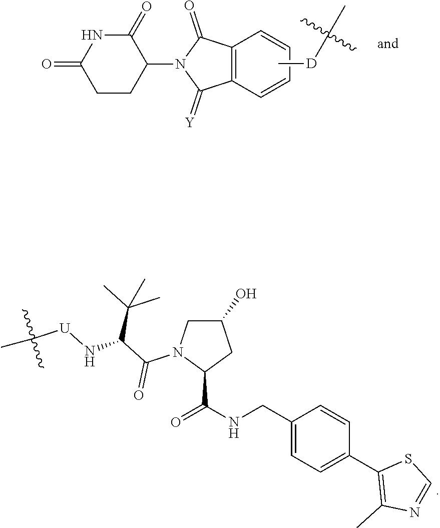

37. The compound of claim 1, or pharmaceutically acceptable salt thereof, wherein the compound is selected from

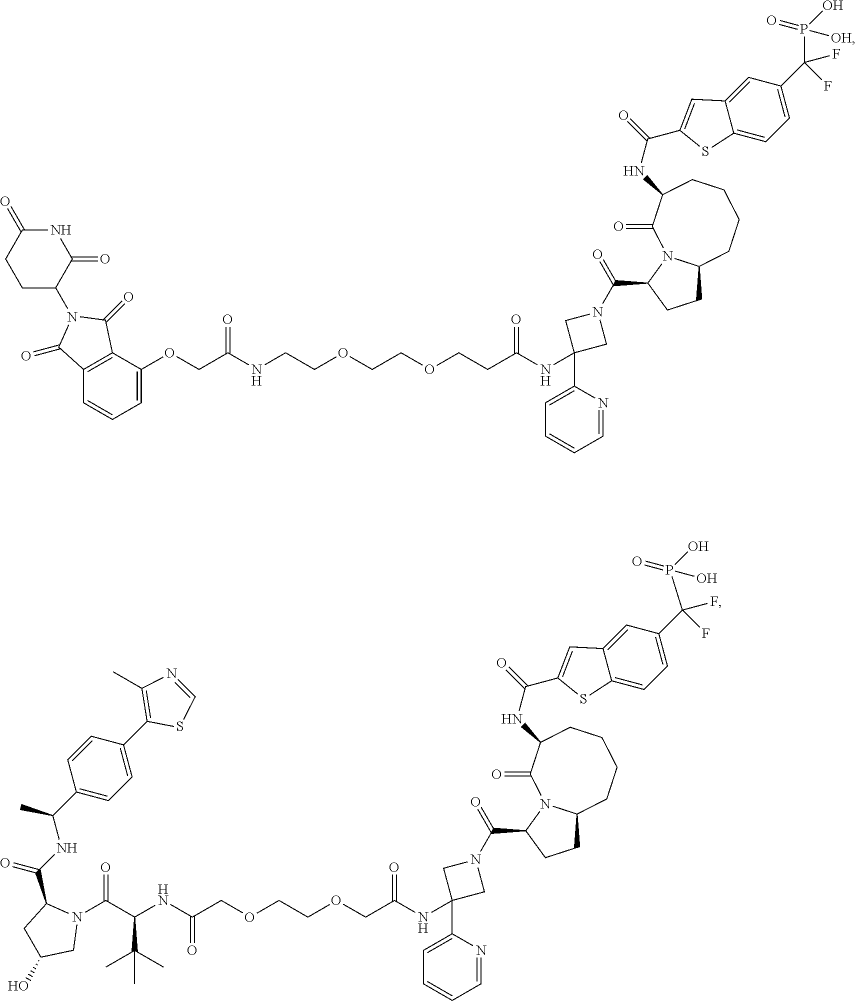 [SMILES: O=C(COc1cccc2c1C(=O)N(C1CCC(=O)NC1=O)C2=O)NCCOCCOCCC(=O)NC1(c2ccccn2)CN(C(=O)[C@@H]2CC[C@@H]3CCCC[C@H](NC(=O)c4cc5cc(C(F)(F)P(=O)(O)O)ccc5s4)C(=O)N32)C1 ; Cc1ncsc1-c1ccc([C@H](C)NC(=O)[C@@H]2C[C@@H](O)CN2C(=O)[C@@H](NC(=O)COCCOCC(=O)NC2(c3ccccn3)CN(C(=O)[C@@H]3CC[C@@H]4CCCC[C@H](NC(=O)c5cc6cc(C(F)(F)P(=O)(O)O)ccc6s5)C(=O)N43)C2)C(C)(C)C)cc1]

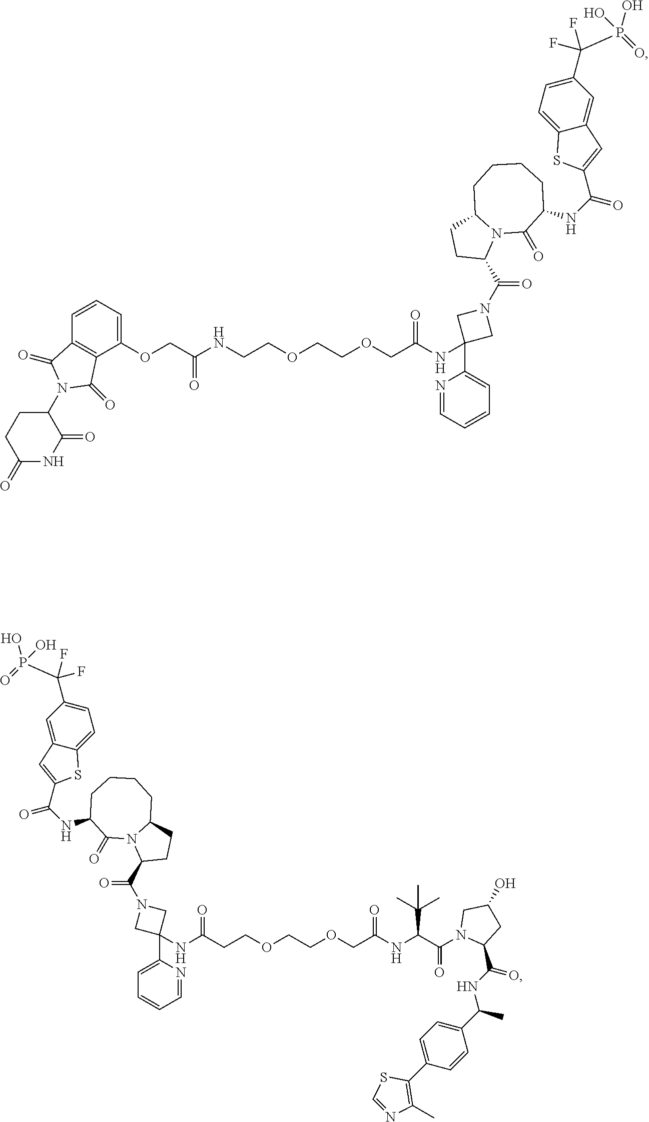 [SMILES: O=C(COc1cccc2c1C(=O)N(C1CCC(=O)NC1=O)C2=O)NCCOCCOCC(=O)NC1(c2ccccn2)CN(C(=O)[C@@H]2CC[C@@H]3CCCC[C@H](NC(=O)c4cc5cc(C(F)(F)P(=O)(O)O)ccc5s4)C(=O)N32)C1 ; Cc1ncsc1-c1ccc([C@H](C)NC(=O)[C@@H]2C[C@@H](O)CN2C(=O)[C@@H](NC(=O)COCCOCCC(=O)NC2(c3ccccn3)CN(C(=O)[C@@H]3CC[C@@H]4CCCC[C@H](NC(=O)c5cc6cc(C(F)(F)P(=O)(O)O)ccc6s5)C(=O)N43)C2)C(C)(C)C)cc1]

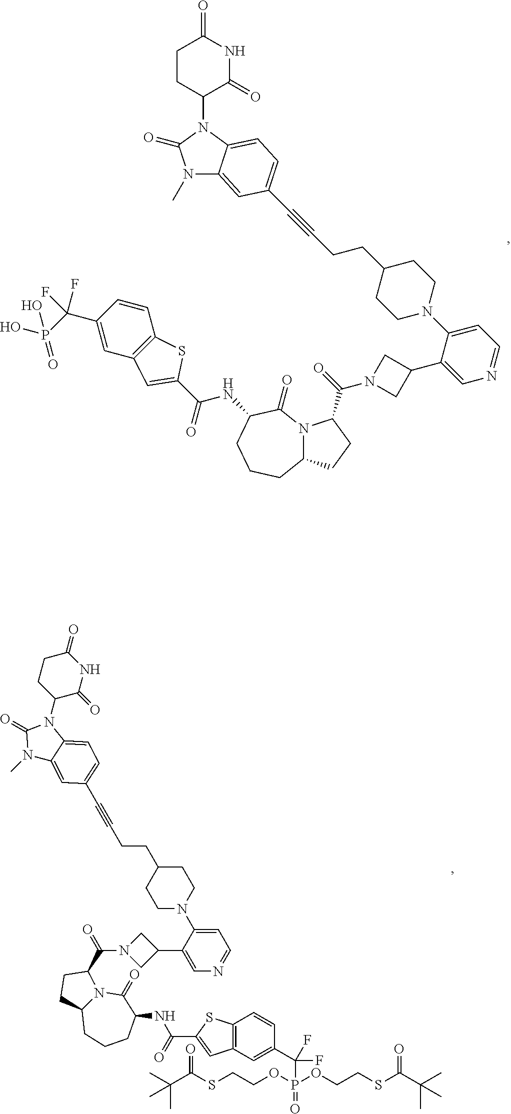 [SMILES: Cn1c(=O)n(C2CCC(=O)NC2=O)c2ccc(C#CCCC3CCN(c4ccncc4C4CN(C(=O)[C@@H]5CC[C@@H]6CCC[C@H](NC(=O)c7cc8cc(C(F)(F)P(=O)(O)O)ccc8s7)C(=O)N65)C4)CC3)cc21 ; Cn1c(=O)n(C2CCC(=O)NC2=O)c2ccc(C#CCCC3CCN(c4ccncc4C4CN(C(=O)[C@@H]5CC[C@@H]6CCC[C@H](NC(=O)c7cc8cc(C(F)(F)P(=O)(OCCSC(=O)C(C)(C)C)OCCSC(=O)C(C)(C)C)ccc8s7)C(=O)N65)C4)CC3)cc21]

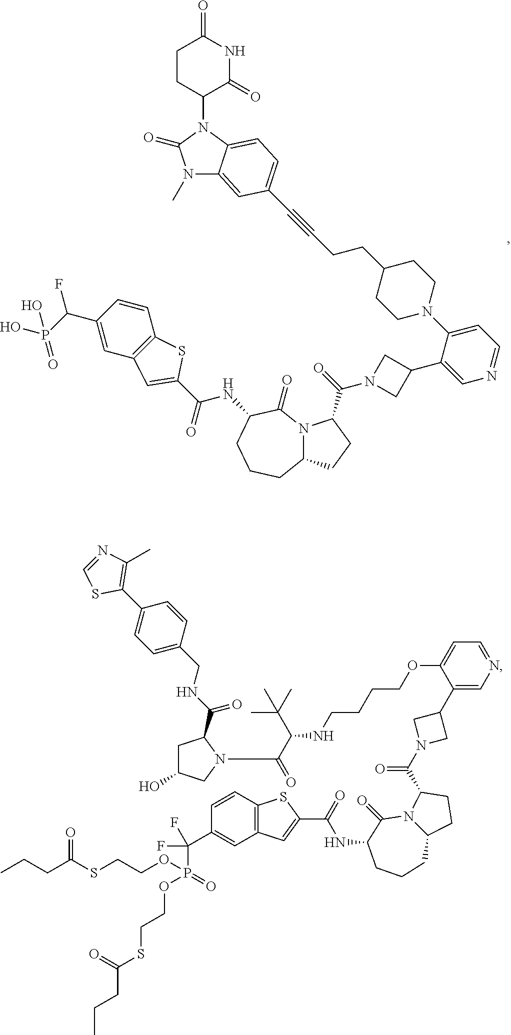 [SMILES: Cn1c(=O)n(C2CCC(=O)NC2=O)c2ccc(C#CCCC3CCN(c4ccncc4C4CN(C(=O)[C@@H]5CC[C@@H]6CCC[C@H](NC(=O)c7cc8cc(C(F)P(=O)(O)O)ccc8s7)C(=O)N65)C4)CC3)cc21 ; CCCC(=O)SCCOP(=O)(OCCSC(=O)CCC)C(F)(F)c1ccc2sc(C(=O)N[C@H]3CCC[C@H]4CC[C@@H](C(=O)N5CC(c6cnccc6OCCCCN[C@H](C(=O)N6C[C@H](O)C[C@H]6C(=O)NCc6ccc(-c7scnc7C)cc6)C(C)(C)C)C5)N4C3=O)cc2c1]

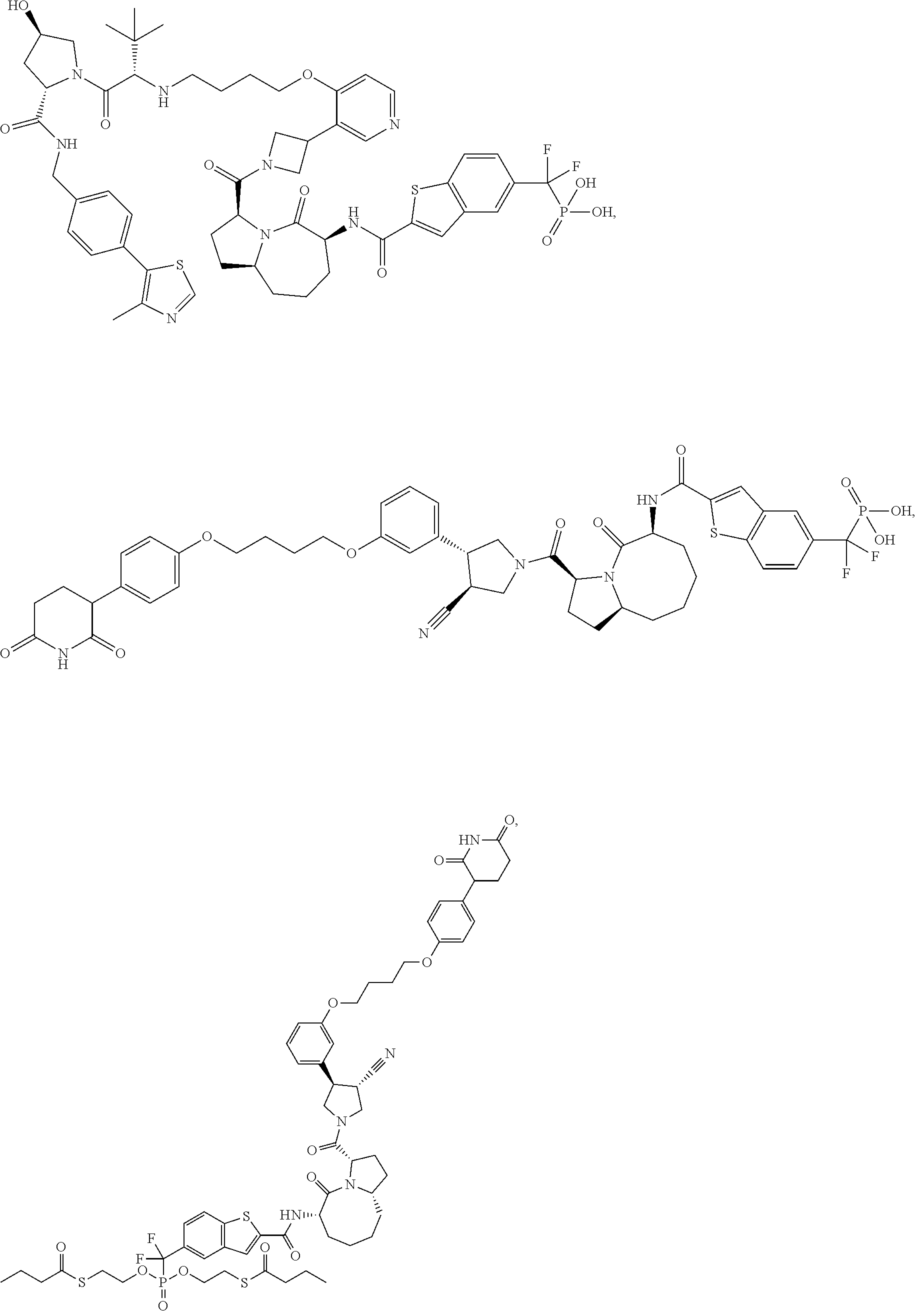 [SMILES: Cc1ncsc1-c1ccc(CNC(=O)[C@@H]2C[C@@H](O)CN2C(=O)[C@@H](NCCCCOc2ccncc2C2CN(C(=O)[C@@H]3CC[C@@H]4CCC[C@H](NC(=O)c5cc6cc(C(F)(F)P(=O)(O)O)ccc6s5)C(=O)N43)C2)C(C)(C)C)cc1 ; N#C[C@@H]1CN(C(=O)[C@@H]2CC[C@@H]3CCCC[C@H](NC(=O)c4cc5cc(C(F)(F)P(=O)(O)O)ccc5s4)C(=O)N32)C[C@H]1c1cccc(OCCCCOc2ccc(C3CCC(=O)NC3=O)cc2)c1 ; CCCC(=O)SCCOP(=O)(OCCSC(=O)CCC)C(F)(F)c1ccc2sc(C(=O)N[C@H]3CCCC[C@H]4CC[C@@H](C(=O)N5C[C@@H](C#N)[C@H](c6cccc(OCCCCOc7ccc(C8CCC(=O)NC8=O)cc7)c6)C5)N4C3=O)cc2c1]

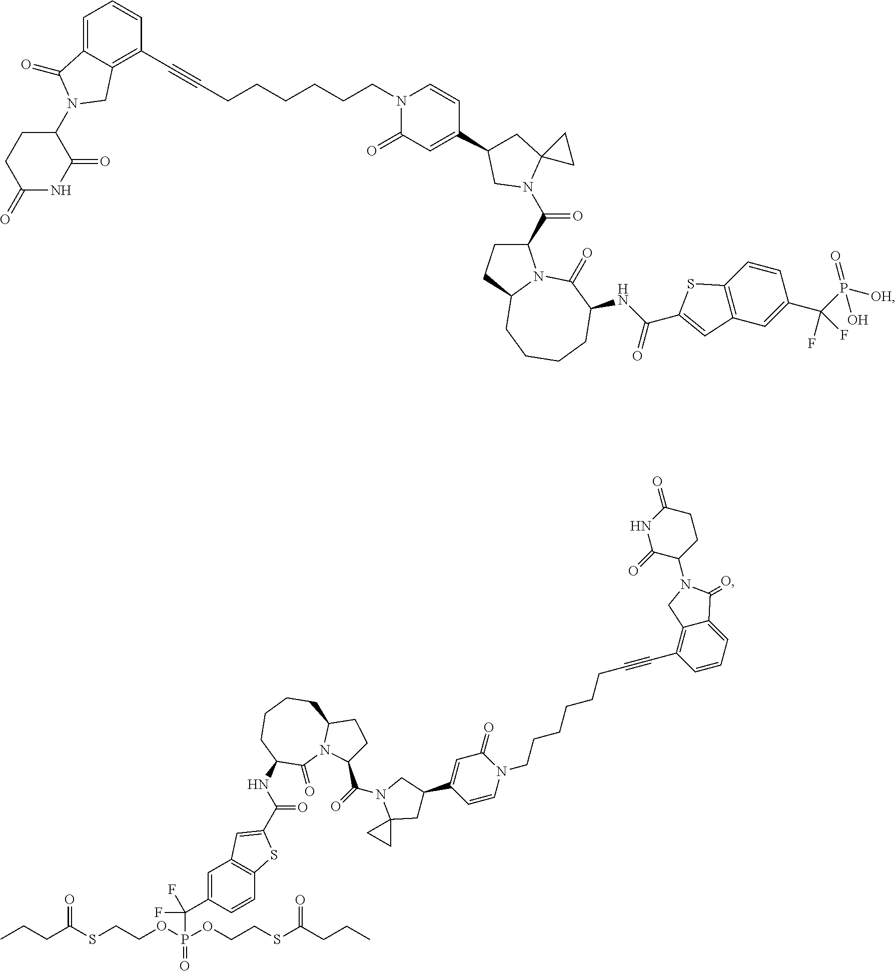 [SMILES: O=C1CCC(N2Cc3c(C#CCCCCCCn4ccc([C@@H]5CN(C(=O)[C@@H]6CC[C@@H]7CCCC[C@H](NC(=O)c8cc9cc(C(F)(F)P(=O)(O)O)ccc9s8)C(=O)N76)C6(CC6)C5)cc4=O)cccc3C2=O)C(=O)N1 ; CCCC(=O)SCCOP(=O)(OCCSC(=O)CCC)C(F)(F)c1ccc2sc(C(=O)N[C@H]3CCCC[C@H]4CC[C@@H](C(=O)N5C[C@@H](c6ccn(CCCCCCC#Cc7cccc8c7CN(C7CCC(=O)NC7=O)C8=O)c(=O)c6)CC56CC6)N4C3=O)cc2c1]

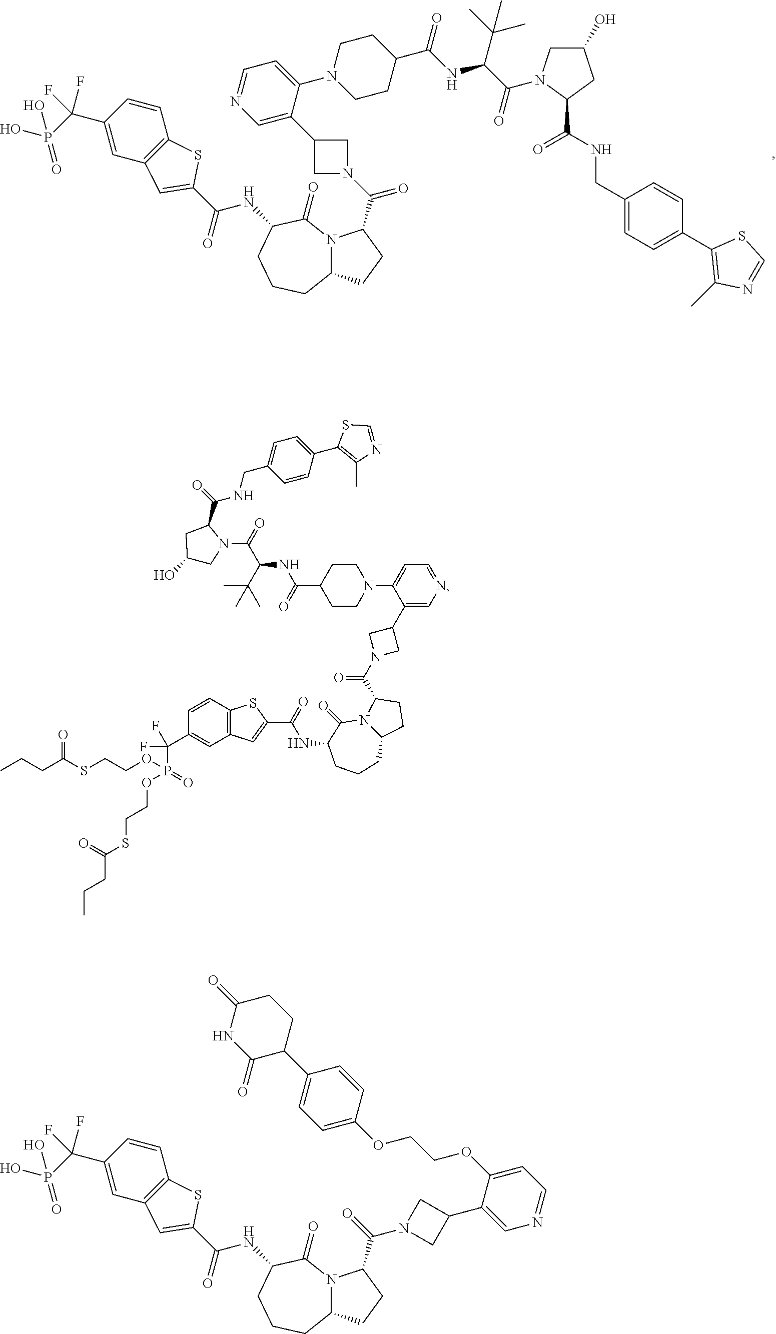 [SMILES: Cc1ncsc1-c1ccc(CNC(=O)[C@@H]2C[C@@H](O)CN2C(=O)[C@@H](NC(=O)C2CCN(c3ccncc3C3CN(C(=O)[C@@H]4CC[C@@H]5CCC[C@H](NC(=O)c6cc7cc(C(F)(F)P(=O)(O)O)ccc7s6)C(=O)N54)C3)CC2)C(C)(C)C)cc1 ; CCCC(=O)SCCOP(=O)(OCCSC(=O)CCC)C(F)(F)c1ccc2sc(C(=O)N[C@H]3CCC[C@H]4CC[C@@H](C(=O)N5CC(c6cnccc6N6CCC(C(=O)N[C@H](C(=O)N7C[C@H](O)C[C@H]7C(=O)NCc7ccc(-c8scnc8C)cc7)C(C)(C)C)CC6)C5)N4C3=O)cc2c1 ; O=C1CCC(c2ccc(OCCOc3ccncc3C3CN(C(=O)[C@@H]4CC[C@@H]5CCC[C@H](NC(=O)c6cc7cc(C(F)(F)P(=O)(O)O)ccc7s6)C(=O)N54)C3)cc2)C(=O)N1]

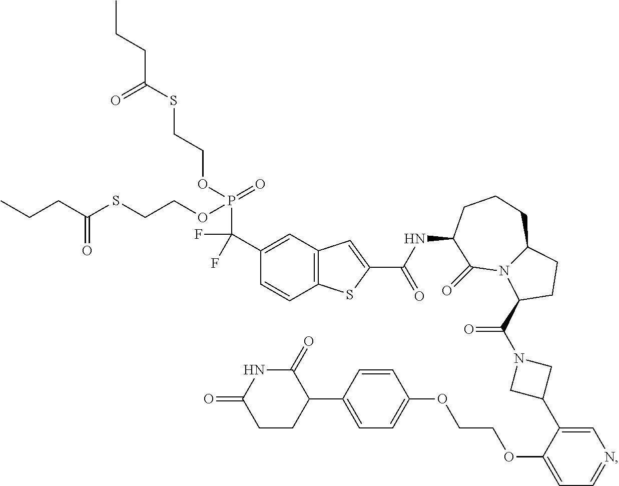 [SMILES: CCCC(=O)SCCOP(=O)(OCCSC(=O)CCC)C(F)(F)c1ccc2sc(C(=O)N[C@H]3CCC[C@H]4CC[C@@H](C(=O)N5CC(c6cnccc6OCCOc6ccc(C7CCC(=O)NC7=O)cc6)C5)N4C3=O)cc2c1]

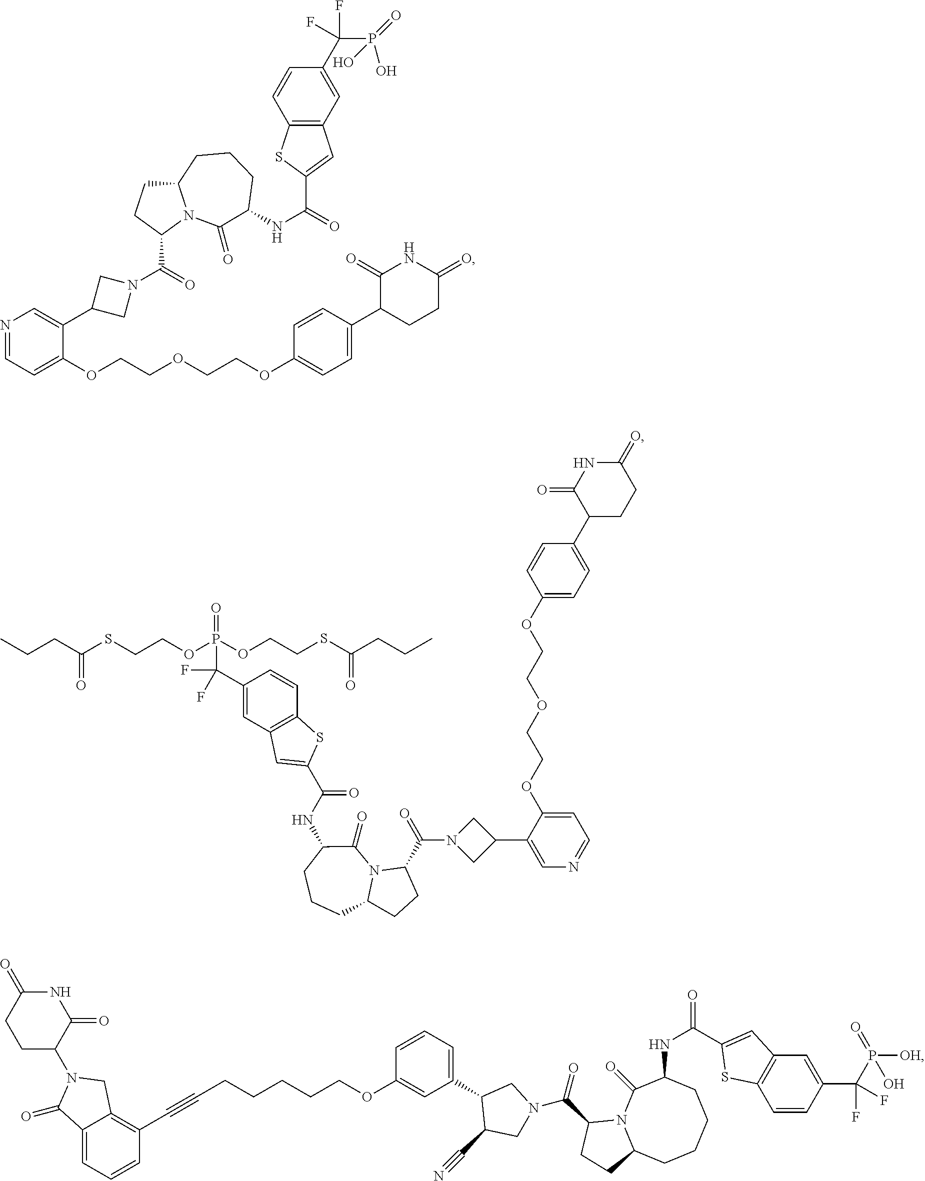 [SMILES: O=C1CCC(c2ccc(OCCOCCOc3ccncc3C3CN(C(=O)[C@@H]4CC[C@@H]5CCC[C@H](NC(=O)c6cc7cc(C(F)(F)P(=O)(O)O)ccc7s6)C(=O)N54)C3)cc2)C(=O)N1 ; CCCC(=O)SCCOP(=O)(OCCSC(=O)CCC)C(F)(F)c1ccc2sc(C(=O)N[C@H]3CCC[C@H]4CC[C@@H](C(=O)N5CC(c6cnccc6OCCOCCOc6ccc(C7CCC(=O)NC7=O)cc6)C5)N4C3=O)cc2c1 ; N#C[C@@H]1CN(C(=O)[C@@H]2CC[C@@H]3CCCC[C@H](NC(=O)c4cc5cc(C(F)(F)P(=O)(O)O)ccc5s4)C(=O)N32)C[C@H]1c1cccc(OCCCCCC#Cc2cccc3c2CN(C2CCC(=O)NC2=O)C3=O)c1]

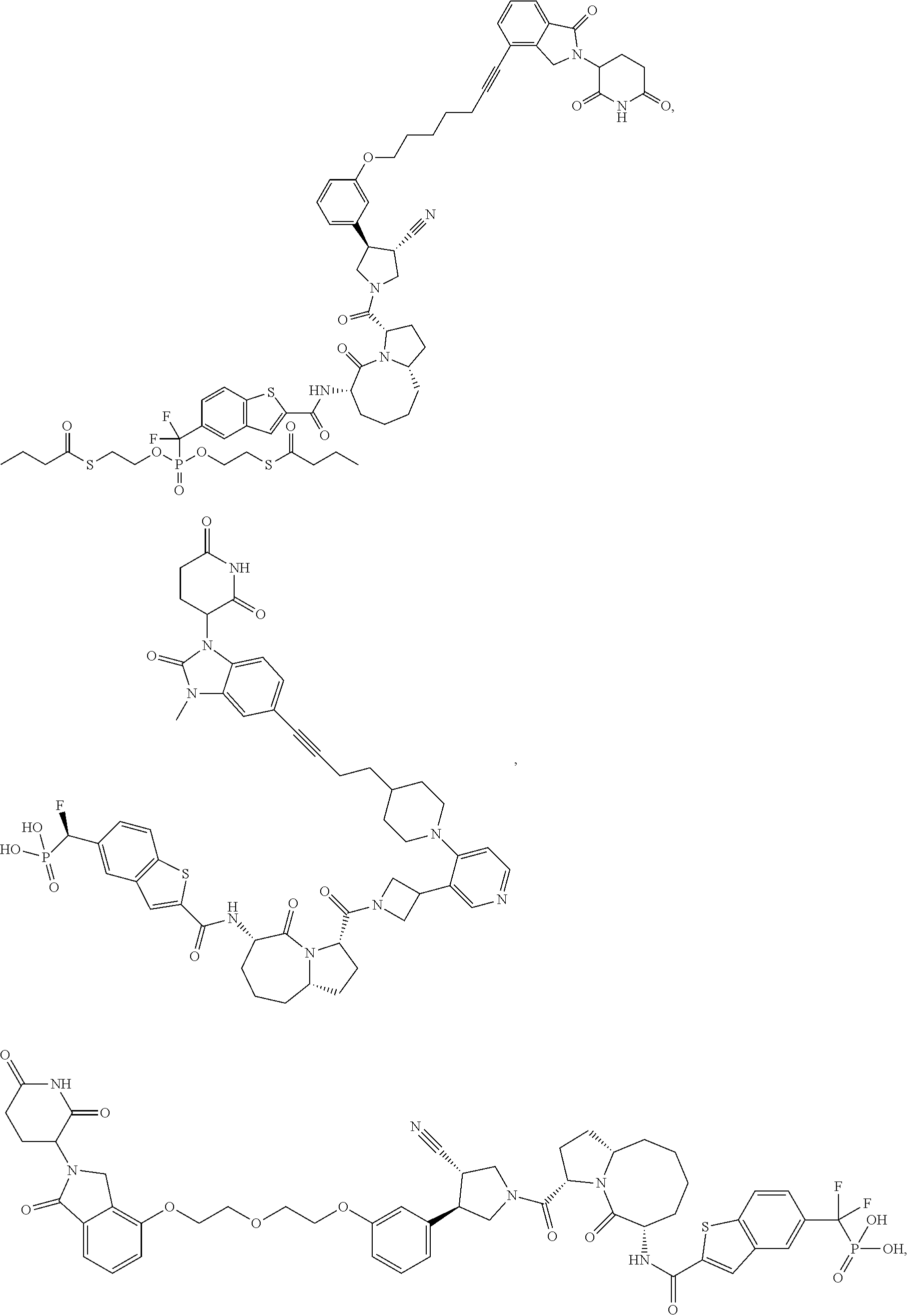

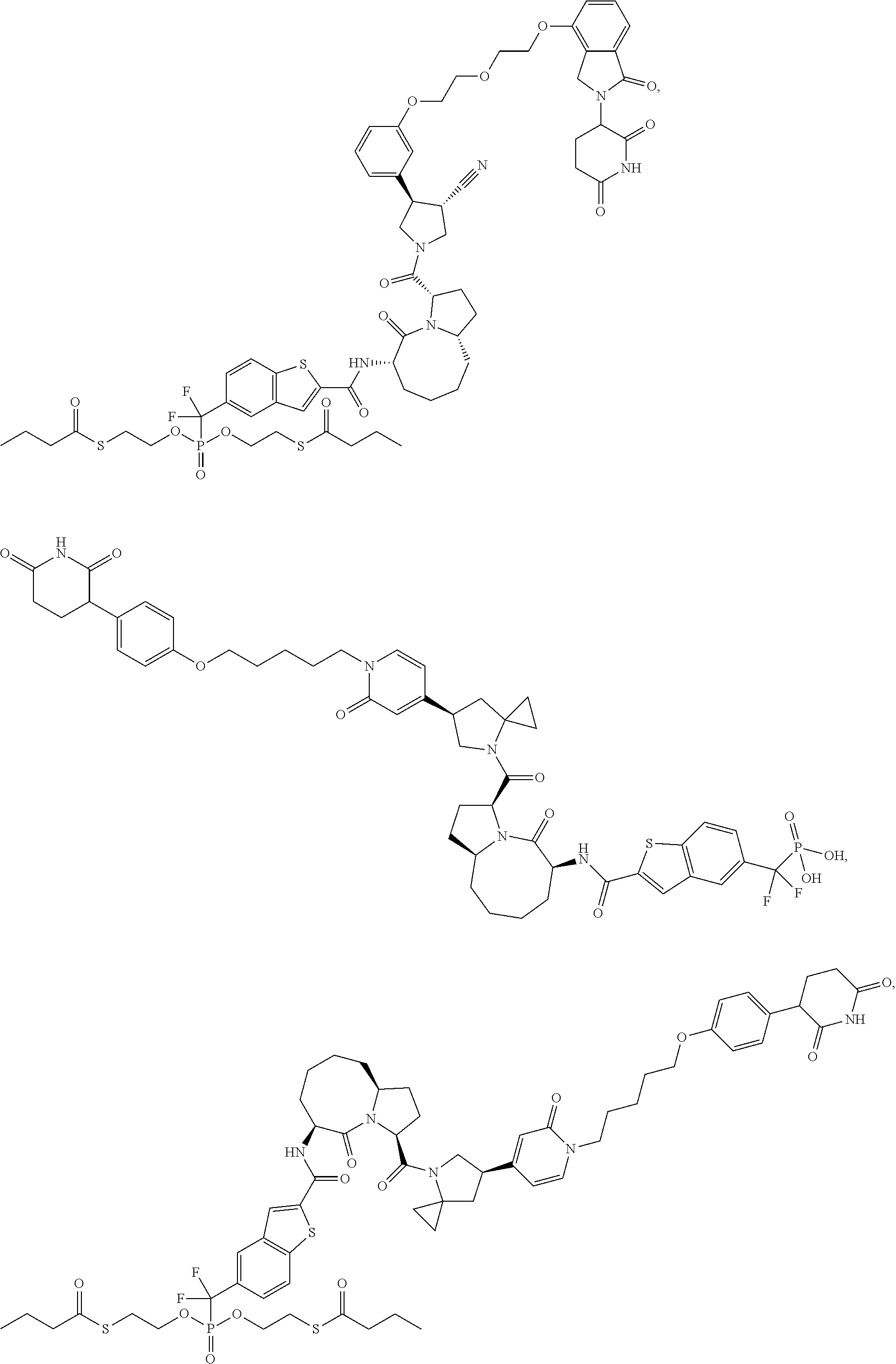 [SMILES: CCCC(=O)SCCOP(=O)(OCCSC(=O)CCC)C(F)(F)c1ccc2sc(C(=O)N[C@H]3CCCC[C@H]4CC[C@@H](C(=O)N5C[C@@H](C#N)[C@H](c6cccc(OCCOCCOc7cccc8c7CN(C7CCC(=O)NC7=O)C8=O)c6)C5)N4C3=O)cc2c1 ; O=C1CCC(c2ccc(OCCCCCn3ccc([C@@H]4CN(C(=O)[C@@H]5CC[C@@H]6CCCC[C@H](NC(=O)c7cc8cc(C(F)(F)P(=O)(O)O)ccc8s7)C(=O)N65)C5(CC5)C4)cc3=O)cc2)C(=O)N1 ; CCCC(=O)SCCOP(=O)(OCCSC(=O)CCC)C(F)(F)c1ccc2sc(C(=O)N[C@H]3CCCC[C@H]4CC[C@@H](C(=O)N5C[C@@H](c6ccn(CCCCCOc7ccc(C8CCC(=O)NC8=O)cc7)c(=O)c6)CC56CC6)N4C3=O)cc2c1]

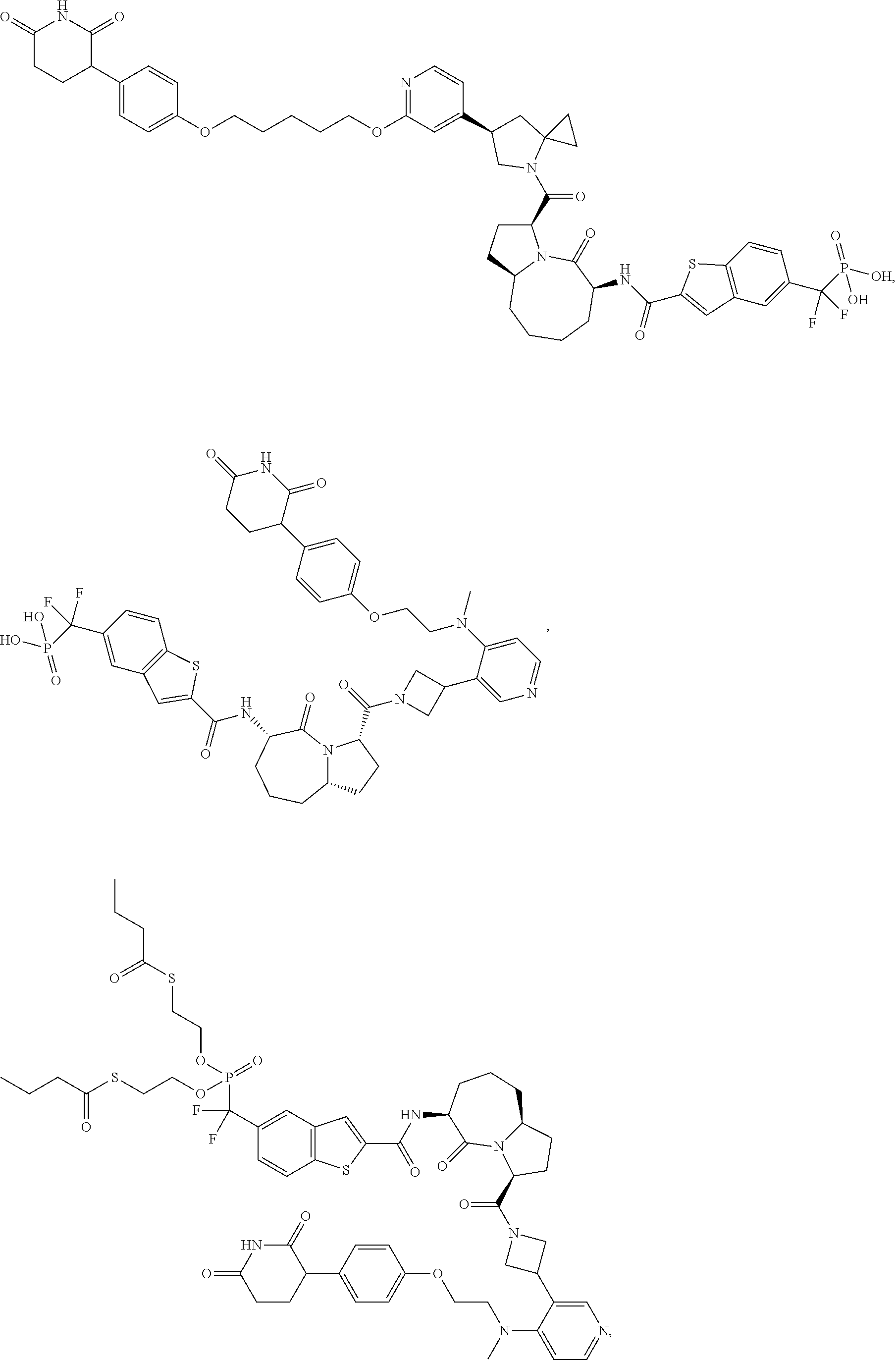 [SMILES: O=C1CCC(c2ccc(OCCCCCOc3cc([C@@H]4CN(C(=O)[C@@H]5CC[C@@H]6CCCC[C@H](NC(=O)c7cc8cc(C(F)(F)P(=O)(O)O)ccc8s7)C(=O)N65)C5(CC5)C4)ccn3)cc2)C(=O)N1 ; CN(CCOc1ccc(C2CCC(=O)NC2=O)cc1)c1ccncc1C1CN(C(=O)[C@@H]2CC[C@@H]3CCC[C@H](NC(=O)c4cc5cc(C(F)(F)P(=O)(O)O)ccc5s4)C(=O)N32)C1 ; CCCC(=O)SCCOP(=O)(OCCSC(=O)CCC)C(F)(F)c1ccc2sc(C(=O)N[C@H]3CCC[C@H]4CC[C@@H](C(=O)N5CC(c6cnccc6N(C)CCOc6ccc(C7CCC(=O)NC7=O)cc6)C5)N4C3=O)cc2c1]

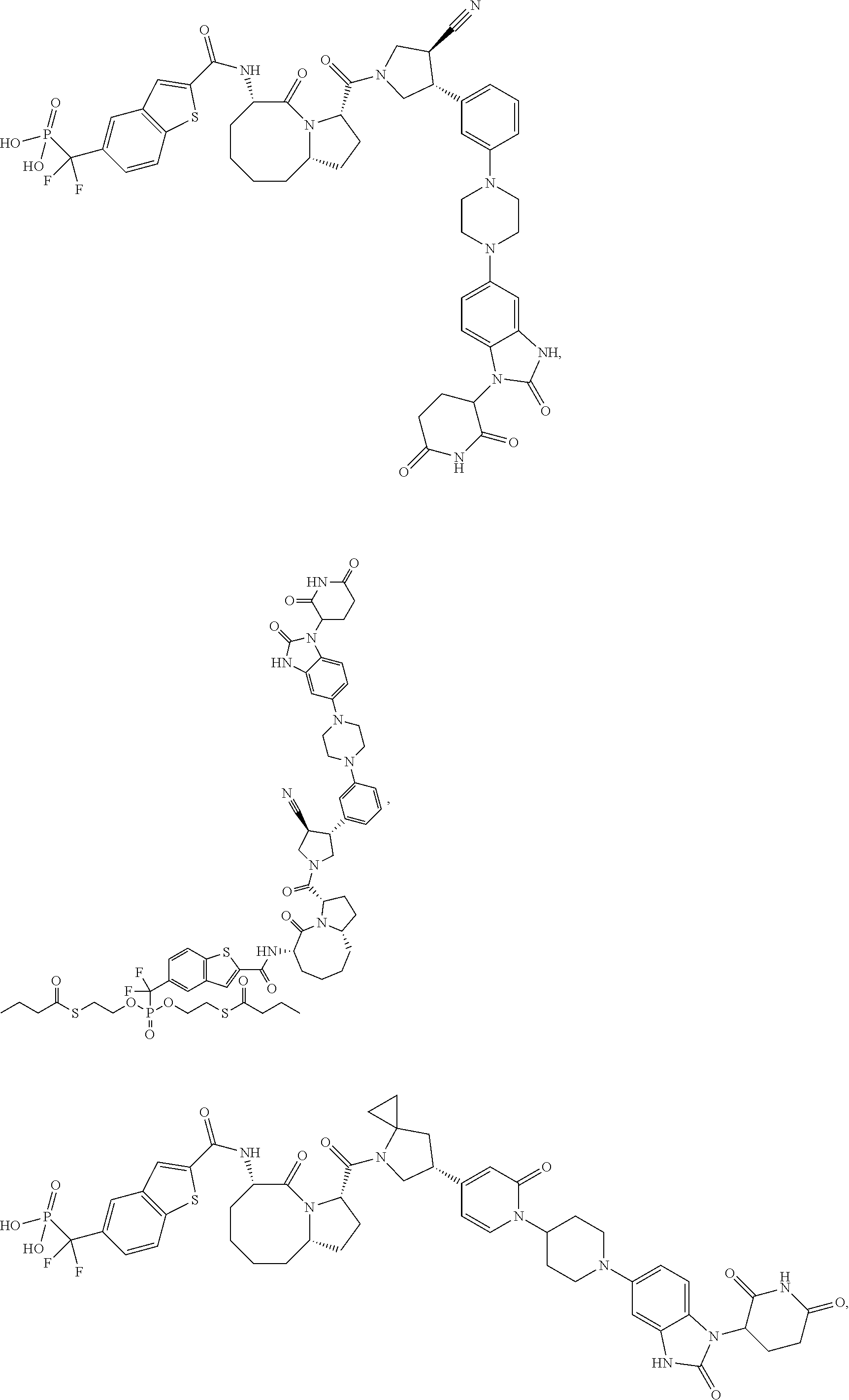 [SMILES: N#C[C@@H]1CN(C(=O)[C@@H]2CC[C@@H]3CCCC[C@H](NC(=O)c4cc5cc(C(F)(F)P(=O)(O)O)ccc5s4)C(=O)N32)C[C@H]1c1cccc(N2CCN(c3ccc4c(c3)[nH]c(=O)n4C3CCC(=O)NC3=O)CC2)c1 ; CCCC(=O)SCCOP(=O)(OCCSC(=O)CCC)C(F)(F)c1ccc2sc(C(=O)N[C@H]3CCCC[C@H]4CC[C@@H](C(=O)N5C[C@@H](C#N)[C@H](c6cccc(N7CCN(c8ccc9c(c8)[nH]c(=O)n9C8CCC(=O)NC8=O)CC7)c6)C5)N4C3=O)cc2c1 ; O=C1CCC(n2c(=O)[nH]c3cc(N4CCC(n5ccc([C@@H]6CN(C(=O)[C@@H]7CC[C@@H]8CCCC[C@H](NC(=O)c9cc%10cc(C(F)(F)P(=O)(O)O)ccc%10s9)C(=O)N87)C7(CC7)C6)cc5=O)CC4)ccc32)C(=O)N1]

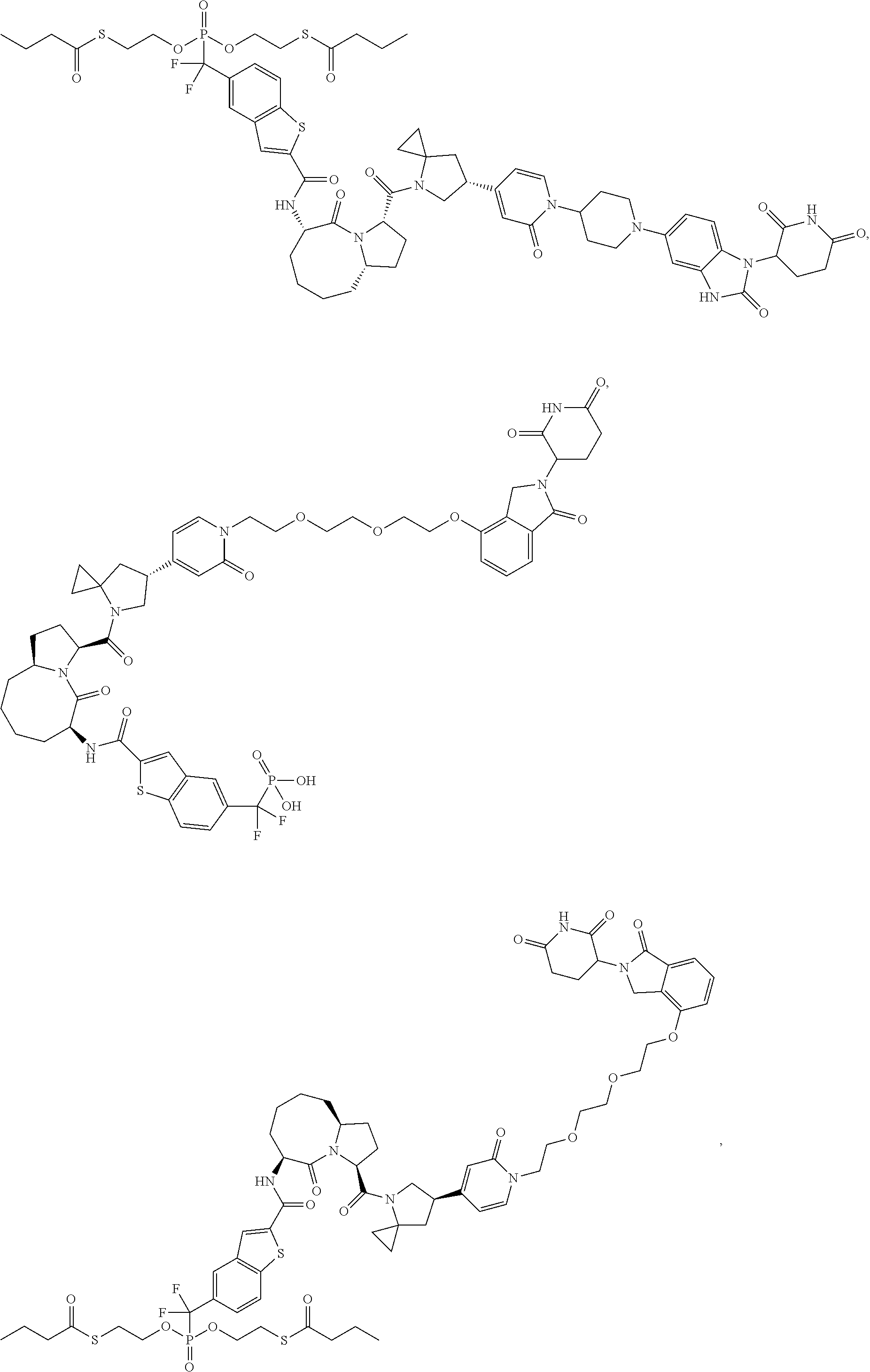 [SMILES: CCCC(=O)SCCOP(=O)(OCCSC(=O)CCC)C(F)(F)c1ccc2sc(C(=O)N[C@H]3CCCC[C@H]4CC[C@@H](C(=O)N5C[C@@H](c6ccn(C7CCN(c8ccc9c(c8)[nH]c(=O)n9C8CCC(=O)NC8=O)CC7)c(=O)c6)CC56CC6)N4C3=O)cc2c1 ; O=C1CCC(N2Cc3c(OCCOCCOCCn4ccc([C@@H]5CN(C(=O)[C@@H]6CC[C@@H]7CCCC[C@H](NC(=O)c8cc9cc(C(F)(F)P(=O)(O)O)ccc9s8)C(=O)N76)C6(CC6)C5)cc4=O)cccc3C2=O)C(=O)N1 ; CCCC(=O)SCCOP(=O)(OCCSC(=O)CCC)C(F)(F)c1ccc2sc(C(=O)N[C@H]3CCCC[C@H]4CC[C@@H](C(=O)N5C[C@@H](c6ccn(CCOCCOCCOc7cccc8c7CN(C7CCC(=O)NC7=O)C8=O)c(=O)c6)CC56CC6)N4C3=O)cc2c1]

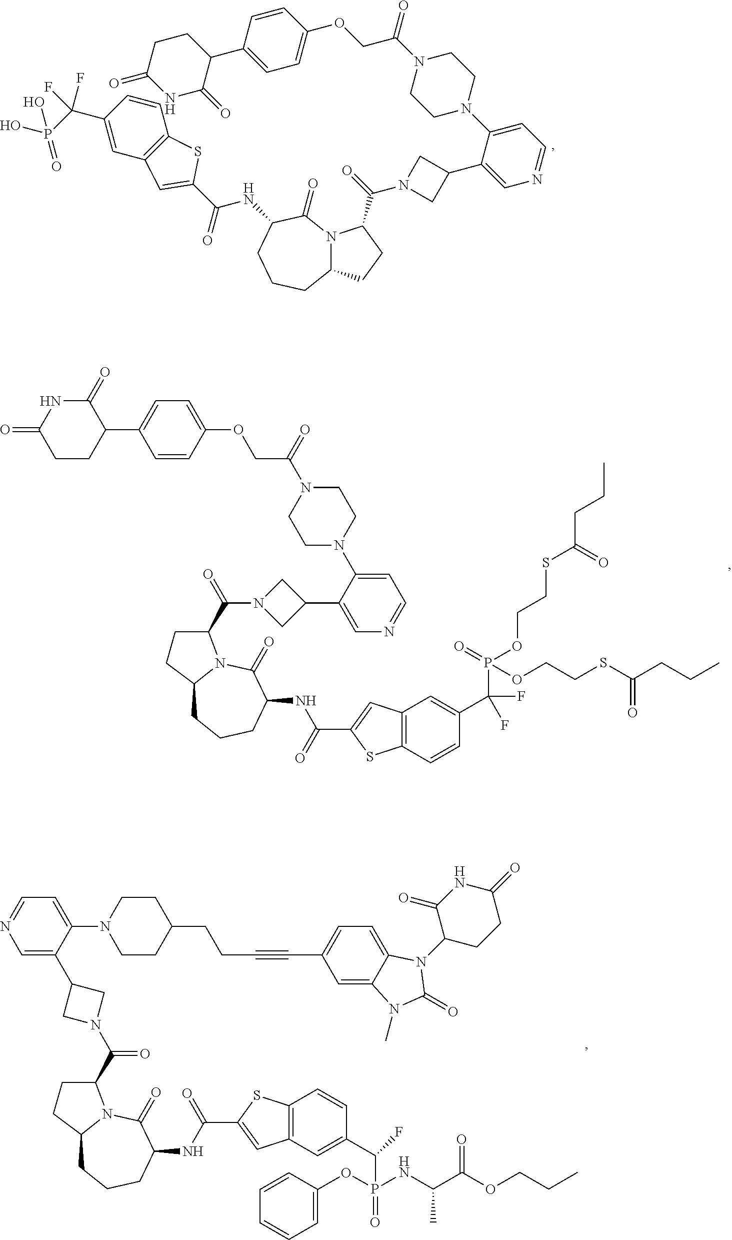 [SMILES: O=C1CCC(c2ccc(OCC(=O)N3CCN(c4ccncc4C4CN(C(=O)[C@@H]5CC[C@@H]6CCC[C@H](NC(=O)c7cc8cc(C(F)(F)P(=O)(O)O)ccc8s7)C(=O)N65)C4)CC3)cc2)C(=O)N1 ; CCCC(=O)SCCOP(=O)(OCCSC(=O)CCC)C(F)(F)c1ccc2sc(C(=O)N[C@H]3CCC[C@H]4CC[C@@H](C(=O)N5CC(c6cnccc6N6CCN(C(=O)COc7ccc(C8CCC(=O)NC8=O)cc7)CC6)C5)N4C3=O)cc2c1 ; CCCOC(=O)[C@H](C)NP(=O)(Oc1ccccc1)[C@@H](F)c1ccc2sc(C(=O)N[C@H]3CCC[C@H]4CC[C@@H](C(=O)N5CC(c6cnccc6N6CCC(CCC#Cc7ccc8c(c7)n(C)c(=O)n8C7CCC(=O)NC7=O)CC6)C5)N4C3=O)cc2c1]

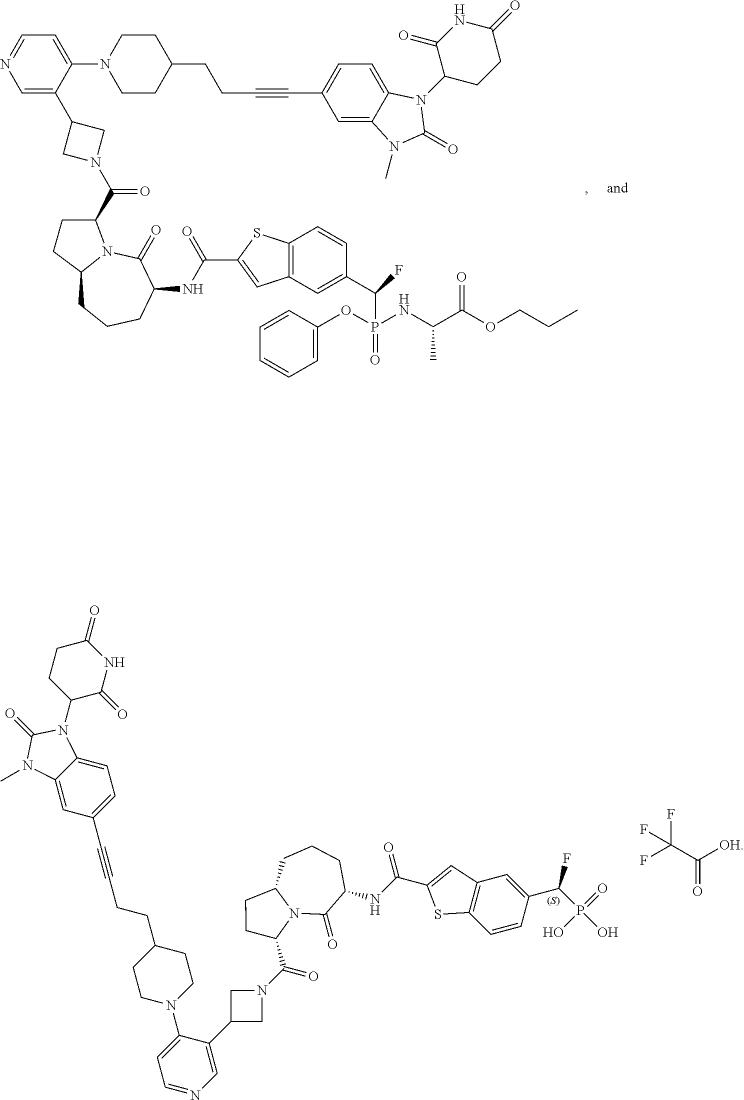

38. A pharmaceutically acceptable composition comprising the compound of any one of claims 1 to 37, or a pharmaceutically acceptable salt thereof; and a pharmaceutically acceptable carrier.

39. A method of treating a condition responsive to the modulation of STAT3 or STAT6 in a subject comprising administering to the subject a therapeutically effective amount of the compound of any one of claims 1 to 37 or a pharmaceutically acceptable salt thereof, or the pharmaceutically acceptable composition of claim 38.
